(19) United States
(12) Patent Application Publication
(10) Pub. No.: US 2021/0246115 A1
(43) Pub. Date: Aug. 12, 2021

(54) ANTI-INFECTIVE HETEROCYCLIC COMPOUNDS AND USES THEREOF

(71) Applicant: Bioimics AB, Uppsala (SE)

(72) Inventors: Leif Kirsebom, Uppsala (SE); Ram Shankar Upadhayaya, Uppsala (SE); Raghava Reddy Kethiri, Bangalore (IN); Anders Virtanen, Uppsala (SE)

(21) Appl. No.: 16/760,855

(22) PCT Filed: Nov. 5, 2018
(86) PCT No.: PCT/SE2018/051126

§ 371 Date: Apr. 30, 2020

(30) Foreign Application Priority Data
Nov. 3, 2017 (SE) 1751369-8

(51) Int. Cl. C07D 401/12; A61P 31/04; C07H 15/26; C07D 209/14; C07D 403/12; C07D 417/12; C07D 405/12; C07D 471/04; C07D 405/06; C07D 417/06; C07D 413/12; C07D 401/06; C07D 401/14; C07D 417/14; C07D 487/04; C07D 401/04; C07D 403/04; C07D 209/24; C07F 9/6558; C07F 9/572; C07D 231/56; C07D 405/14

(52) U.S. Cl. CPC C07D 401/12; C07D 405/14; C07H 15/26; C07D 209/14; C07D 403/12; C07D 417/12; C07D 405/12; C07D 471/04; C07D 405/06; C07D 417/06; C07D 413/12; C07D 401/06; C07D 401/14; C07D 417/14; C07D 487/04; C07D 401/04; C07D 403/04; C07D 209/24; C07F 9/65583; C07F 9/5728; C07D 231/56; A61P 31/04

(57) ABSTRACT

The present invention relates to heterocyclic compounds of Formula F-I useful as anti-infective agents. The present invention further relates to a method of treating an infection by administering such compounds, and to pharmaceutical compositions comprising such compounds.

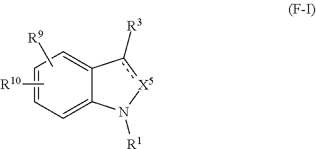

### FIELD OF THE INVENTION

The present invention relates to heterocyclic compounds useful as anti-infective agents. The present invention further relates to a method of treating an infection by administering such a compound. The present invention further relates to pharmaceutical compositions comprising such compounds.

### BACKGROUND ART

Antimicrobial resistance is an increasingly serious threat to global public health. New resistance mechanisms emerge and spread globally, threatening the effective prevention and treatment of a range of infections caused by bacteria, parasites and fungi.

A number of examples can be provided to illustrate the threat posed. In 2013 there was approximately half a million new cases of multi-drug resistant tuberculosis. Resistance to artemisinin-based combination therapies, which are the best available treatment for Plasmodium falciparum malaria, has been detected in the Greater Mekong subregion. Highly resistant bacteria such as MRSA cause a high percentage of hospital-acquired infections and it is also beginning to spread in the community. Patients with such drug-resistant infections have an increased risk of inferior clinical outcomes and death as compared to patients infected with non-resistant bacteria. Ten countries have reported cases where gonorrhoea was untreatable due to resistance to the treatments of last resort antibiotics (3^{rd} generation cephalosporins). Thus, gonorrhoea may soon become untreatable.

This emphasize an increased and urgent need for new anti-infective agents for use in therapy.

### SUMMARY OF THE INVENTION

The object of the invention is thus to provide compounds useful for the treatment or prevention of infection. A further object is to provide a method of treating an infection, such as a bacterial, fungal or parasitic infection.

These objects are achieved by compounds as disclosed by the appended claims.

The compounds have the formula F-I:

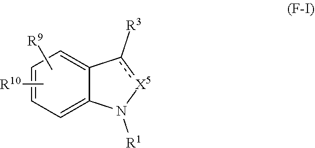

or a pharmaceutically acceptable salt thereof

wherein

X⁵ is selected from CH, CMe, C═O, and N;

[symbol] F denotes a double bond when X⁵ is CH, CMe or N, and a single bond when X⁵ is C═O;

R¹ is selected from the group consisting of
R², —(CH₂)ₘ—R², —C(O)—R², and —CHMe-R²;

R² is selected from the group consisting of
phenyl optionally substituted with one of more groups selected from -halo and —C₁₋₃ alkyl,
C₃₋₁₀ cycloalkyl wherein the cycloalkyl group is mono-, bi- or polycyclic and is optionally substituted with one of more groups selected from —F and -Me,
C₁₋₁₀ alkyl wherein the alkyl group is straight or branched,
C₂₋₁₀ alkenyl wherein the alkenyl group is straight or branched, and
heterocyclyl wherein the heterocyclyl group is a 5- or 6-membered aliphatic heterocycle;

R³ is selected from the group consisting of
CH(R⁴)—(CH₂)ₙ—C(O)NR⁵R⁶,
CH(R⁴)—(CH₂)ₙ—NHR⁵,
CH(R⁴)—(CH₂)ₙ—NR⁵R⁶,
CH(R⁴)—(CH₂)ₙ—CH(NH₂)—C(O)NR⁵R⁶,
C(O)—NR⁵R⁶,
(CH₂)ₙ-Cy-NR⁵R⁶, and
CH(R⁴)—(CH₂)ₙ—OR⁶

R⁴ is selected from the group consisting of
C₁₋₆ alkyl, wherein the alkyl group is straight or branched,
C₃₋₆ cycloalkyl,
phenyl optionally substituted with one or more groups selected from -halo, —C₁₋₃ alkyl, —C₁₋₃ perhaloalkyl, —C₁₋₃ alkoxy, —C₁₋₃ perhaloalkoxy, and -hydroxyl,
benzyl, optionally substituted with one or more groups selected from -halo, —C₁₋₃ alkyl, —C₁₋₃ perhaloalkyl, —C₁₋₃ alkoxy, —C₁₋₃ perhaloalkoxy, and -hydroxyl,
heterocyclyl wherein the heterocyclyl group is a 5- or 6-membered aliphatic or aromatic heterocycle, optionally benzo-fused, and optionally substituted with one of more groups selected from -benzyl, -halo, —C₁₋₃ alkyl, —C₁₋₃ perhaloalkyl, —C₁₋₃ alkoxy, —C₁₋₃ perthaloalkoxy, and -hydroxyl;

R⁵ is selected from the group consisting of
—H,
benzyl, optionally substituted with with one of more groups selected from -halo and —C₁₋₃ alkyl,
C₁₋₆ alkyl,
acetyl,
CN, and
(CH₂)₃—NH₂;

or

R⁴ and R⁵ together with the atoms to which they are bound form a heteroaliphatic ring;

R⁶ is selected from the group consisting of
C₁₋₃ alkyl, optionally substituted with one or more R⁷ groups
C₀₋₃ alkyl-cycloalkyl, wherein the cycloalkyl group is a 3-6 membered monocyclic cycloalkyl optionally substituted with one or more R⁷ groups,
C(O)-cycloalkyl, wherein the cycloalkyl group is a 3-6 membered monocyclic cycloalkyl optionally substituted with one or more R⁷ groups,
C₁₋₃₃ alkyl-heterocyclyl, wherein the heterocyclyl group is a 5- or 6-membered aliphatic or aromatic heterocycle, optionally benzo-fused, and is optionally substituted with one or more R⁷ groups,
C₁₋₃ alkyl-phenyl, wherein the phenyl group is optionally substituted with one or more R⁷ groups,
C(O)—(CH₂)ₚ—NH—(CH₂)ᵣ—phenyl, wherein the phenyl group is optionally substituted with one or more R⁷ groups;

or

R⁵ and R⁶ together with the atom to which they are bound form a heteroaliphatic ring optionally substituted with one or more R⁷ groups;

R⁷ is selected from the group consisting of -halo, —C₁₋₃ alkyl, —C₁₋₃ alkoxy, phenyl, hydroxy, —CH₂OH, -oxo, —C(O)Me, —SO₂Me, —SO₂Ph optionally substituted with —F, mono- or di-C₁₋₃ alkyl amine, —C(O)—NH₂, —NH—C(O)—NH₂, —C(═NH)—NH₂, —NH—C(═NH)—NH₂, —(CH₂)ₛ—NH₂, piperidine, piperazine, morpholine, —(CH₂)ₜ—NH—P(O)(OEt)₂, —C(O)—NH—R⁸, and -phenoxy optionally substituted with —Cl;

R⁸ is selected from the group consisting of —OH, -(amino)cyclohexyl, -pyrrolidinylethyl, and -methylpiperazinylethyl;

R⁹ and R¹⁰ are each independently selected from the group consisting of —H, -halo, —C₁₋₃ alkyl, —C₁₋₃ perfluoroalkyl, C₂₋₃ alkoxy, —C₁₋₃ perfluoroalkoxy, —NO₂, —OH, —CN, —CO₂H, —CO₂Me, —CO₂NH₂, —CH₂NH₂, -Cy, -pyridinyl, -tetrahydropyridinyl, -pyrazinyl optionally substituted with -Me, and -phenyl optionally substituted with -halo, —C₁₋₃ alkyl, —C₁₋₃ perfluoroalkyl, —C₁₋₃ alkoxy, —C₁₋₃ perfluoroalkoxy; and

wherein m, n, p, r, s and t are each independently selected from 0, 1 and 2.

Disclosed herein are also compounds of Formula I:

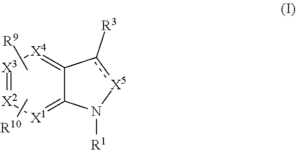

or a pharmaceutically acceptable salt thereof

wherein

each of X¹, X², X³, and X⁴ is independently selected from C and N;

X⁵ is selected from CH CMe C═O, and N;

R¹ is selected from the group consisting of
H, —R², —(CH₂)ₘ—R², —C(O)—R², and —CHMe-R²;

R² is selected from the group consisting of
phenyl optionally substituted with one of more groups selected from -halo and —C₁₋₃ alkyl,
C₃₋₁₀ cycloalkyl wherein the cycloalkyl group is mono-, bi- or polycyclic and is optionally substituted with one of more groups selected from —F and -Me,
C₁₋₁₀ alkyl wherein the alkyl group is straight or branched,
C₂₋₁₀ alkenyl wherein the alkenyl group is straight or branched, and
heterocyclyl wherein the heterocyclyl group is a 5- or 6-membered aliphatic heterocycle;

R³ is selected from the group consisting of
CH(R⁴)—(CH₂)ₙ—C(O)NR⁵R⁶,
CH(R⁴)—(CH₂)ₙ—NHR⁵,
CH(R⁴)—(CH₂)ₙ—NR⁵R⁶,
CH(R⁴)—(CH₂)ₙ—CH(NH₂)—C(O)NR⁵R⁶,
C(O)—NR⁵R⁶,
(CH₂)ₙ-Cy-NR⁵R⁶, and
CH(R⁴)—(CH₂)ₙ—OR⁶;

R⁴ is selected from the group consisting of
H,
C₁₋₆ alkyl, wherein the alkyl group is straight or branched,
C₃₋₆ cycloalkyl,
phenyl optionally substituted with one or more groups selected from -halo, —C₁₋₃ alkyl, —C₁₋₃ perhaloalkyl, —C₁₋₃ alkoxy, —C₁₋₃ perhaloalkoxy, and -hydroxyl,
benzyl, optionally substituted with one or more groups selected from -halo, —C₁₋₃ alkyl, —C₁₋₃ perhaloalkyl, —C₁₋₃ alkoxy, —C₁₋₃ perhaloalkoxy, and -hydroxyl,
heterocyclyl wherein the heterocyclyl group is a 5- or 6-membered aliphatic or aromatic heterocycle, optionally benzo-fused, and optionally substituted with one of more groups selected from -benzyl, -halo, —C₁₋₃ alkyl, —C₁₋₃ perhaloalkyl, —C₁₋₃ alkoxy, —C₁₋₃ perthaloalkoxy, and -hydroxyl;

R⁵ is selected from the group consisting of
H,
benzyl, optionally substituted with with one of more groups selected from -halo and —C₁₋₃ alkyl,
C₁₋₆ alkyl,
acetyl,
CN, and
(CH₂)₃—NH₂;

or

wherein R⁴ and R⁵ together with the atoms to which they are bound form a heteroaliphatic ring;

R⁶ is selected from the group consisting of
C₁₋₃ alkyl, optionally substituted with one or more R⁷ groups
C₀₋₃ alkyl-cycloalkyl, wherein the cycloalkyl group is a 3-6 membered monocyclic cycloalkyl optionally substituted with one or more R⁷ groups,
C(O)-cycloalkyl, wherein the cycloalkyl group is a 3-6 membered monocyclic cycloalkyl optionally substituted with one or more R⁷ groups,
C₀₋₃ alkyl-heterocyclyl, wherein the heterocyclyl group is a 5- or 6-membered aliphatic or aromatic heterocycle, optionally benzo-fused, and is optionally substituted with one or more R⁷ groups,
C₁₋₃ alkyl-phenyl, wherein the phenyl group is optionally substituted with one or more R⁷ groups,
C(O)—(CH₂)ₚ—NH—(CH₂)ᵣ—phenyl, wherein the phenyl group is optionally substituted with one or more R⁷ groups;

or

wherein R⁵ and R⁶ together with the atoms to which they are bound form a heteroaliphatic ring optionally substituted with one or more R⁷ groups;

R⁷ is selected from the group consisting of -halo, —C₁₋₃ alkyl, —C₁₋₃ alkoxy, phenyl, hydroxy, —CH₂OH, -oxo, —C(O)Me, —SO₂Me, —SO₂Ph optionally substituted with —F, mono- or di-C₁₋₃ alkyl amine, —C(O)—NH₂, —NH—C(O)—NH₂, —C(═NH)—NH₂, —NH—C(═NH)—NH₂, —(CH₂)ₛ—NH₂, piperidine, piperazine, morpholine, —(CH₂)ₜ—NH—P(O)(OEt)₂, —C(O)—NH—R, and -phenoxy optionally substituted with —Cl;

R⁸ is selected from the group consisting of —OH, -(amino)cyclohexyl, -pyrrolidinylethyl, and -methylpiperazinylethyl;

R⁹ and R¹⁰ are each independently selected from the group consisting of —H, -halo, —C₁₋₃ alkyl, —C₁₋₃ perfluoroalkyl, —C₁₋₃ alkoxy, —C₁₋₃ perfluoroalkoxy, —NO₂, —OH, —CN, —CO₂H, —CO₂Me, —CO₂NH₂, —CH₂NH₂, -Cy, -pyridinyl, -tetrahydropyridinyl, -pyrazinyl optionally substituted with -Me, and -phenyl optionally substituted with -halo, —C₁₋₃ alkyl, —C₁₋₃ perfluoroalkyl, —C₁₋₃ alkoxy, —C₁₋₃ perfluoroalkoxy; and

wherein m, n, p, r, s and t are each independently selected from 0, 1 or 2.

Compounds, or salts therefore, as defined by Formula I and F-I can be used in the treatment or prevention of infection, especially bacterial infection.

Without wishing to be bound by theory, it is thought that the compounds disclosed above achieve their antimicrobial effect at least in part by inhibition of RNase P. RNase P is a ribonucleoprotein complex present in all living cells and in bacteria RNase P is involved in the processing of RNA transcripts such as removal of 5′ leader sequences from tRNA precursors. In bacteria, RNase P consists of one RNA subunit and a small basic protein, and it has been shown that the catalytic activity is associated with its RNA subunit. RNase P is potentially a good drug target since RNase P is indispensable for bacterial viability and the architecture of RNase P differs between bacteria and eukaryote. For example, the important P-15 loop in bacteria is a good target for antibacterial drug design since it is not present in human (eukaryotic) RNase P RNA.

The compounds of formula F-I may belong to a subset of compounds having Formula F-II:

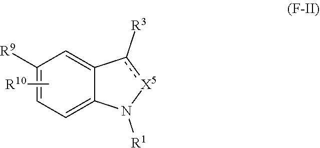

or a pharmaceutically acceptable salt thereof

wherein

X⁵ is selected from CH CMe C═O, and N;

[symbol] denotes a double bond when X⁵ is CH, CMe or N, and a single bond when X⁵ is C═O;

R¹ is selected from the group consisting of

—R², —(CH₂)ₘ—R², —C(O)—R², and —CHMe-R²;

R² is selected from the group consisting of
phenyl optionally substituted with one of more groups selected from —F and -Me,
C₃₋₁₀ cycloalkyl wherein the cycloalkyl group is cyclopropyl, cycloheptyl, bicycloheptyl or adamantanyl, optionally substituted with one of more groups selected from —F and -Me,
C₁₋₁₀ alkyl wherein the alkyl group is ethyl, isopropyl or octyl,
C₂₋₁₀ alkenyl wherein the alkenyl group is straight or branched, and
heterocyclyl wherein the heterocyclyl group is piperidyl or hetrahydropyranyl;

R³ is selected from the group consisting of
CH(R⁴)—(CH₂)ₙ—C(O)NR⁵R⁶,
CH(R⁴)—(CH₂)ₙ—NHR⁵,
CH(R⁴)—(CH₂)ₙ—NR⁵R⁶,
CH₂—CH(NH₂)—C(O)NR⁵R⁶,
C(O)—NR⁵R⁶,
Cy-NR⁵R⁶, and
CH(R⁴)—(CH₂)ₙ—OR⁶;

R⁴ is selected from the group consisting of
C₁₋₆ alkyl, wherein the alkyl group is straight or branched,
C₃₋₆ cycloalkyl selected from the group consisting of cyclopropyl, cyclopentyl and cyclohexyl,
phenyl optionally substituted with one or more groups selected from —F, —Cl, -Me, -iPr, —CF₃, —OMe, OCF₃,
benzyl, optionally substituted with one or more methyl groups,
heterocyclyl wherein the heterocyclyl group is imidazolyl, thiazolyl, pyridinyl, piperidinyl, tetrahydropyranyl, quinolinyl or isoquinolinyl, and is optionally substituted with one of more groups selected from -benzyl, and -hydroxyl;

R⁵ is selected from the group consisting of
H,
benzyl, optionally substituted with with one of more groups selected from —F and -Me,
C₁₋₂ alkyl,
acetyl,
CN, and
(CH₂)₃—NH₂;

or

R⁴ and R⁵ together with the atoms to which they are bound form a 6-membered heteroaliphatic ring;

R⁶ is selected from the group consisting of
C₁₋₃ alkyl, optionally substituted with one or more R⁷ groups
C₀₋₃ alkyl-cycloalkyl, wherein the cycloalkyl group is cyclopropyl, cyclopentyl or cyclohexyl, optionally substituted with one or more R⁷ groups,
C(O)-cycloalkyl, wherein the cycloalkyl group is cyclopropyl, cyclopentyl or cyclohexyl, optionally substituted with one or more R⁷ groups,
C₀₋₃ alkyl-heterocyclyl, wherein the heterocyclyl group is pyrrolidinyl, pyridinyl, imidazolyl, thiazolyl, piperidinyl, furanyl, benzodioxolanyl, oxazolyl, morpholinyl or tetrahydropyranyl, and is optionally substituted with one or more R⁷ groups,
C₁₋₃ alkyl-phenyl, wherein the phenyl group is optionally substituted with one or more R⁷ groups,
C(O)—(CH₂)ₚ—NH—(CH₂)-phenyl, wherein the phenyl group is optionally substituted with one or more R⁷ groups;

or

R⁵ and R⁶ together with the atom to which they are bound form a 6-membered heteroaliphatic ring which ring is optionally substituted with one or more R⁷ groups;

R⁷ is selected from the group consisting of methyl, fluoro, bromo, phenyl, hydroxy, —CH₂OH, -oxo, methoxy, —C(O)Me, , —SO₂Me, —SO₂Ph optionally substituted with —F, —NH₂, —NHMe, —NMe₂, —C(O)—NH₂, —NH—C(O)—NH₂, —C(═NH)—NH₂, —NH—C(═NH)—NH₂, —(CH₂)ₛ—NH₂, piperidine, piperazine, morpholine, —(CH₂)ₜ—NH—P(O)(OEt)₂, —C(O)NH—R⁸, and phenoxy optionally substituted with —Cl;

R⁸ is selected from the group consisting of —OH, -(amino)cyclohexyl, -pyrrolidinylethyl, and -methylpiperazinylethyl;

R⁹ is selected from the group consisting of —H, —F, —Br, —NO₂, —OH, —CN, —CO₂H, —CO₂Me, —CO₂NH₂, —CH₂NH₂, -Cy, -pyridinyl, -tetrahydropyridinyl, -pyrazinyl optionally substituted with -Me, and -phenyl optionally substituted with —Cl, -Me, —CF₃, —OMe or —OCF₃;

R¹⁰ is —H or —Br; and

m, n, p, r, s and t are each dependently selected from 0, 1 and 2.

The compounds of formula F-I and F-II may belong to a subset of compounds having Formula F-III:

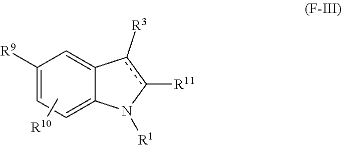

or a pharmaceutically acceptable salt thereof

wherein R¹¹ is —H, -Me or -oxo;

[symbol] denotes a double bond when R¹¹ is —H or -Me, and a single bond when R¹¹ is oxo.

The compounds of formula F-I, F-II and F-III may belong to a subset of compounds having Formula F-IV:

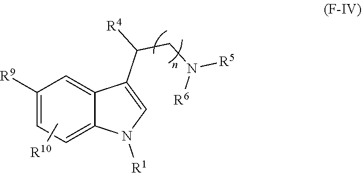

or a pharmaceutically acceptable salt thereof.

The compounds of formula F-I, F-II and F-III may belong to a subset of compounds having Formula F-V:

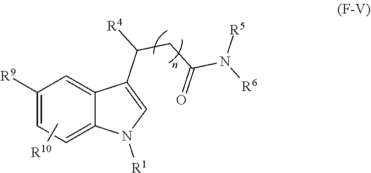

or a pharmaceutically acceptable salt thereof.

The compounds of Formula F-I, F-II and F-III may belong to a subset of compounds having a Formula VI:

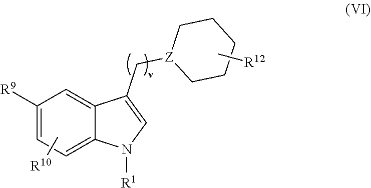

or a pharmaceutically acceptable salt thereof,

wherein v is 0 or 1

Z is selected from CH or N

and wherein

whenever Z is CH R¹² is —NR⁵R⁶ and

whenever Z is N, R¹² is selected from an R⁷ group comprising at least one N atom.

The compounds of any one of Formulas F-I, F-II, F-III, F-IV and F-V may belong to a subset of compounds wherein:

R¹ is cyclohexanyl or n-octyl;

n is 2;

R⁴ is selected from the group consisting of -Cy, -PhOCF₃ and pentan-3-yl;

R⁵ is H;

R⁶ is —(CH₂)₃—NH₂ or -Cy-NH₂;

R⁹ is —H or —CN; and

R¹⁰ is H.

The compound of Formula VI may belong to a subset of compounds wherein:

R¹ is cyclohexanyl or n-octyl;

R⁹ is —H or —CN; and

R¹⁰ is H.

The compounds of Formula I may belong to a subset of compounds having a Formula II:

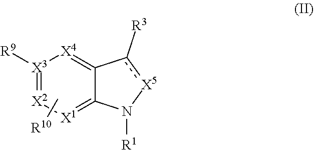

or a pharmaceutically acceptable salt thereof.

Each of X¹, X², X³, and X⁴ may independently be selected from C and N, with the proviso that when X³ is N then X¹ is also N.

X⁵ may be selected from CH, CMe, C═O, and N.

R¹ may be selected from the group consisting of
H, —R², —(CH₂)ₘ—R², —C(O)—R², and —CHMe-R².

R² may be selected from the group consisting of
phenyl optionally substituted with one of more groups selected from —F and -Me,
C₃₋₁₀ cycloalkyl wherein the cycloalkyl group is cyclopropyl, cycloheptyl, bicycloheptyl or adamantanyl, optionally substituted with one of more groups selected from —F and -Me,
C₁₋₁₀ alkyl wherein the alkyl group is ethyl, isopropyl or octyl,
C₂₋₁₀ alkenyl wherein the alkenyl group is straight or branched, and
heterocyclyl wherein the heterocyclyl group is piperidyl or hetrahydropyranyl.

R³ may selected from the group consisting of
CH(R⁴)—(CH₂)ₙ—C(O)NR⁵R⁶,
CH(R⁴)—(CH₂)ₙ—NHR⁵,
CH(R⁴)—(CH₂)ₙ—NR⁵R⁶,
CH₂—CH(NH₂)—C(O)NR⁵R⁶,
C(O)—NR⁵R⁶,
Cy-NR⁵R⁶, and
CH(R⁴)—(CH₂)ₙ—OR⁶.

R⁴ may be selected from the group consisting of
H,
C₁₋₆ alkyl, wherein the alkyl group is straight or branched,
C₃₋₆ cycloalkyl selected from the group consisting of cyclopropyl, cyclopentyl and cyclohexyl,
phenyl optionally substituted with one or more groups selected from —F, —Cl, -Me, -iPr, —CF₃, —OMe, OCF₃,
benzyl, optionally substituted with one or more methyl groups-C₁₋₃ alkyl, and
heterocyclyl wherein the heterocyclyl group is imidazolyl, thiazolyl, pyridinyl, piperidinyl, tetrahydropyranyl, quinolinyl or isoquinolinyl, and is optionally substituted with one of more groups selected from -benzyl, and -hydroxyl.

R⁵ may be selected from the group consisting of
H,
benzyl, optionally substituted with with one of more groups selected from —F and -Me,
C₁₋₂ alkyl,
acetyl,
CN, and
(CH₂)₃—NH₂.

R⁴ and R⁵ together with the atoms to which they are bound may form a 6-membered heteroaliphatic ring.

R⁶ may be selected from the group consisting of
C₁₋₃ alkyl, optionally substituted with one or more R⁷ groups
C₀₋₃ alkyl-cycloalkyl, wherein the cycloalkyl group is cyclopropyl, cyclopentyl or cyclohexyl, optionally substituted with one or more R⁷ groups,
C(O)-cycloalkyl, wherein the cycloalkyl group is cyclopropyl, cyclopentyl or cyclohexyl, optionally substituted with one or more R⁷ groups,
C₀₋₃ alkyl-heterocyclyl, wherein the heterocyclyl group is pyrrolidinyl, pyridinyl, imidazolyl, thiazolyl, piperidinyl, furanyl, benzodioxolanyl, oxazolyl, morpholinyl or tetrahydropyranyl, and is optionally substituted with one or more R⁷ groups,
C₁₋₃ alkyl-phenyl, wherein the phenyl group is optionally substituted with one or more R⁷ groups, and
C(O)—(CH₂)ₚ—NH—(CH₂)ᵣ-phenyl, wherein the phenyl group is optionally substituted with one or more R⁷ groups.

R⁵ and R⁶ together with the atoms to which they are bound may form a 6-membered heteroaliphatic ring optionally substituted with one or more R⁷ groups.

R⁷ may be selected from the group consisting of methyl, fluoro, bromo, phenyl, hydroxy, —CH₂OH, -oxo, methoxy, —C(O)Me, , —SO₂Me, —SO₂Ph optionally substituted with —F, —NH₂, —NHMe, —NMe₂, —C(O)—NH₂, —NH—C(O)—NH₂, —C(═NH)—NH₂, —NH—C(═NH)—NH₂, —(CH₂)ₛ—NH₂, piperidine, piperazine, morpholine, —(CH₂)ₜ—NH—P(O)(OEt)₂, —C(O)NH—R⁸, and phenoxy optionally substituted with —Cl.

R⁸ may be selected from the group consisting of —OH, -(amino)cyclohexyl, -pyrrolidinylethyl, and -methylpiperazinylethyl.

R⁹ may be selected from the group consisting of —H, —F, —Br, —NO₂, —OH, —OMe, —CN, —CO₂H, —CO₂Me, —CO₂NH₂, —CH₂NH₂, -Cy, -pyridinyl, -tetrahydropyridinyl, -pyrazinyl optionally substituted with -Me, and -phenyl optionally substituted with —Cl, -Me, —CF₃, —OMe or —OCF₃.

R¹⁰ may be —H or —Br.

m, n, p, r, s and t may each be independently selected from 0, 1 or 2.

The compounds of Formula I or II may belong to a subset of compounds having a Formula III:

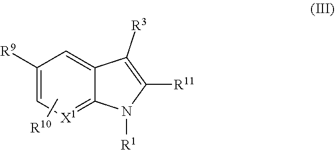

or a pharmaceutically acceptable salt thereof

wherein R¹¹ is —H, -Me or -oxo.

The compounds of Formula I-III may belong to a subset of compounds having a Formula IV:

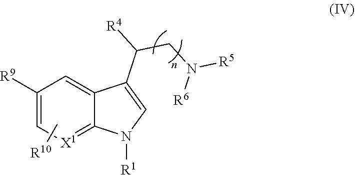

or a pharmaceutically acceptable salt thereof.

The compounds of any one of Formulas I-III may belong to a subset of compounds having a Formula V:

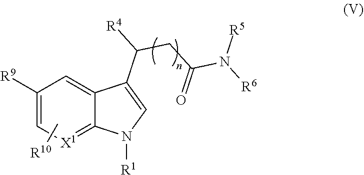

or a pharmaceutically acceptable salt thereof.

The compounds of any one of Formulas I-III may belong to a subset of compounds having a Formula VI:

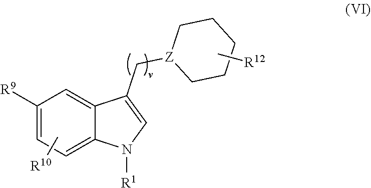

or a pharmaceutically acceptable salt thereof,

wherein v is 0 or 1

Z is selected from CH or N

and wherein

whenever Z is CH R¹² is —NR⁵R⁶ and

whenever Z is N, R is selected from an R⁷ group comprising at least one N atom.

The compounds of any one of Formulas I-VI may belong to a subset of compounds wherein:

R¹ is cyclohexanyl or n-octyl;

n is 2;

R⁴ is selected from the group consisting of -Cy, -PhOCF₃ and pentan-3-yl;

R⁵ is H;

R⁶ is —(CH₂)₃—NH₂ or -Cy-NH₂;

R⁹ is —H or —CN; and

R¹⁰ is H.

The compounds of any one of Formulas I-V may belong to a subset of compounds wherein: each of X¹-X⁴ is C, and X⁵ is CH.

According to another aspect of the present invention, the objects of the invention are achieved by a compound according to Formula F-I, I or II or any subgroup thereof as disclosed above, for use in a method of treatment of the human or animal body by therapy. The therapy may be treatment or prevention of an infection. The infection may be a bacterial, fungal, or parasite infection. The infection may be a bacterial infection caused or complicated by bacteria of a genus selected from Staphylococcus, Enterococcus, Streptococcus, Pseudomonas, Legionella, Klebsiella, Haemophilus, Neisseria, Listeria, Escherichia, Helicobacter and Mycobacterium. The bacterial infection may be caused or complicated by a bacterial species selected from the group: S. aureus, E. faecalis, E. faecium, S. pneumoniae, E. coli, K. pneumoniae, H. influenza, A. baumannii, P. aeruginosa, P. aeruginosa, N. gonorrhoeae, M. fortuitum, M. phlei, and H. pylori. The bacterial infection may be caused or complicated by a bacterial species selected from the group: Neisseria meningitides, Listeria monocytogenes, Legionella pneumophila, Mycobacterium bovis, and Mycobacteria tuberculosis. The bacterial infection may be caused or complicated by a Methicillin-resistant Staphylococcus aureus (MRSA).

According to a further aspect of the present invention, the objects of the invention are achieved by a method of treating an infection which comprises administering to a patient in need thereof a therapeutically effective amount of a compound as disclosed above. The infection may be a bacterial, fungal, or parasite infection. The infection may be a bacterial infection caused or complicated by bacteria of a genus selected from Staphylococcus, Enterococcus, Streptococcus, Pseudomonas, Legionella, Klebsiella, Haemophilus, Neisseria, Listeria, Escherichia, Helicobacter and Mycobacterium. The bacterial infection may be caused or complicated by a bacterial species selected from the group: S. aureus, E. faecalis, E. faecium, S. pneumoniae, E. coli, K. pneumoniae, H. influenza, A. baumannii, P. aeruginosa, P. aeruginosa, N. gonorrhoeae, M. fortuitum, M. phlei, and H. pylori. The bacterial infection may be caused or complicated by a bacterial species selected from the group: Neisseria meningitides, Listeria monocytogenes, Legionella pneumophila, Mycobacterium bovis, and Mycobacteria tuberculosis. The bacterial infection may be caused or complicated by a Methicillin-resistant Staphylococcus aureus.

According to yet another aspect of the present invention, the object of the invention is achieved by use of a compound as disclosed above, or a salt thereof, in inhibition of bacterial RNase P activity.

According to yet a further aspect of the present invention, the object of the invention is achieved by use of a compound as disclosed above, or a salt thereof, as a bactericide.

According to still a further aspect of the present invention, the object of the invention is achieved by a pharmaceutical composition comprising a compound as disclosed above, or a pharmaceutically acceptable salt thereof, in association with a pharmaceutically acceptable excipient, adjuvant, diluent and/or carrier.

Further aspects, objects and advantages are defined in the detailed description below with reference to the appended drawings.

### BRIEF DESCRIPTION OF THE DRAWINGS

For the understanding of the present invention and further objects and advantages of it, the detailed description set out below can be read together with the accompanying drawings.

FIG. 1 shows Scheme 1 for the synthesis of selected compounds according to the present invention.

FIG. 2 shows Scheme 2 for the synthesis of selected compounds according to the present invention.

FIG. 3 shows Scheme 3 for the synthesis of selected compounds according to the present invention.

FIG. 4 shows General Scheme 1 for the synthesis of selected compounds according to the present invention.

FIG. 5 shows a synthetic scheme for the synthesis of 3-(3-((3-aminopropyl) amino)-1-(3-(trifluoromethoxy)phenyl)propyl)-1-cyclohexyl-1H-indole-5-carbonitrile dihydrochloride according to the present invention.

FIG. 6 shows General scheme 2 for the synthesis of selected compounds according to the present invention.

FIG. 7 shows General Scheme 3 for the synthesis of selected compounds according to the present invention.

FIG. 8 shows General Scheme 4 for the synthesis of selected compounds according to the present invention.

FIG. 9 shows General Scheme 5A for the synthesis of selected compounds according to the present invention.

FIG. 10 shows General Scheme 5B for the synthesis of selected compounds according to the present invention.

FIG. 11 shows General Scheme 6 for the synthesis of selected compounds according to the present invention.

FIG. 12 shows a synthetic scheme for the synthesis of N-((1R,4R)-4-aminocyclohexyl)-3-(1-(cyclohexylmethyl)-5-phenyl-1H-indol-3-yl)-3-(m-tolyl) propanamide according to the present invention.

FIG. 13 shows General Scheme 8 for the synthesis of selected compounds according to the present invention.

FIG. 14 shows General Scheme 9 for the synthesis of selected compounds according to the present invention.

FIG. 15 shows General Scheme 10 for the synthesis of selected compounds according to the present invention.

FIG. 16 shows General Scheme 11 for the synthesis of selected compounds according to the present invention.

### DETAILED DESCRIPTION

### General Synthetic Methods

All reactions were carried out under dry nitrogen and or argon atmosphere unless otherwise specified. Unless otherwise stated, all the raw starting materials, solvents, and reagents were purchased from commercial sources (e.g., AVRA Chemicals, Apollo Scientific Limited, Bepharma Ltd., Combi-Blocks Inc., Sigma Aldrich Chemicals Pvt. Ltd., Ultra Labs, Toronto Research Chemicals Inc., Chemical House, RFCL Limited, Spectro Chem Pvt. Ltd., Leonid Chemicals, Loba Chemie, Changzhou Yangyuan, NeoSynth., Rankem, etc.) and used as such without further purification. Alternatively, reagents may be synthesized by procedures known in the literature.

The following abbreviations are used and have the indicated definitions: MHz is megahertz (frequency), m is multiplet, t is triplet, d is doublet, s is singlet, br is broad, CDCl₃ is deutero chloroform, calcd is calculated, min is minutes, h is hours, g is grams, mmol is millimoles, mL is milliliters, N is normality (concentration), M is molarity (concentration), M is micromolar, ee is enantiomeric excess, de is diastereomeric excess, ° C. is degree centigrade, HPLC is High Performance Liquid Chromatography, LC-MS is Liquid Chromatography-Mass Spectroscopy, NMR is Nuclear Magnetic Resonance, TLC is Thin Layer Chromatography, THE is tetrahydrofuran, MeOH is methanol, DCM is dichloromethane, DEA is diethylamine, DMA is dimethylacetamide, DMF is N,N-dimethyl formamide, DMSO is dimethyl sulfoxide, EtOH is ethyl alcohol, EtOAc is ethyl acetate, RT is room temperature, HCl is hydrogen chloride or hydrochloric acid, TFA is trifluoroacetic acid, EtMgBr is ethyl magnesium bromide, n-BuLi is n-butyl lithium, NaHCO₃ is sodium bicarbonate, Na₂CO₃ is sodium carbonate, Na₂SO₄ is sodium sulfate, DCC is N,N-dicyclohexylcarbodiimide, DIPA is diisopropylamine, LDA is lithium diisopropylamine, HOBt is N-hydroxy-benzotriazole, NCS is N-chlorosuccinimide, and TBAB is tetrabutyl ammonium bromide.

Biotage Isolera® One and CombiFlash®(Teledyne Isco) Automated Flash Purification System were used for the purification of crude products using the eluent combination mentioned in the respective procedures. Flash Chromatography was performed using silica gel (60-100, 100-200 and 230-400 mesh) from ChemLabs, with nitrogen and/or compressed air. Preparative thin-layer chromatography was carried out using silica gel (GF 1500 μM 20×20 cm and GF 2000 μM 20×20 cm prep-scored plates from Analtech, Inc. Delaware, USA). Thin-layer chromatography was carried out using pre-coated silica gel sheets (Merck 60 F₂₅₄). Visual detection was performed with ultraviolet light, p-anisaldehyde stain, ninhydrin stain, dinitrophenyl hydrazine stain, potassium permanganate stain, or iodine. Reactions at lower temperature were performed by using cold baths, e.g., H₂O/ice at 0° C., and acetone/dry ice at -78° C. Melting points were determined by using a LabIndia MR-VIS visual melting range apparatus. ¹H NMR spectra were recorded at 400 MHz with a Varian V400 spectrometer, Bruker 400 (unless otherwise noted) at ambient temperature, using tetramethylsilane as internal reference. The chemical shift values are quoted in 6 (parts per million). Mass spectra of all the intermediates and final compounds were recorded using Acquity® UPLC-SQD (Waters) & Agilent 1290 Infinity® with 6150 SQD machines. HPLC spectra were recorded using Agilent 1290 Infinity® UHPLC and Alliance (Waters) systems. LCMS spectra were recorded using Agilent 1200® LCMS/Agilent 1290® UHPLC-SQD with diode array detector (DAD) detection LC-MS instruments using Kinetex C18 (50 mm×2.1 mm×2.7mic) and/orX-terra MS C18 (50 mm×2.1 mm×3.0 micron) columns. The purity of each of the final compounds was detected using Waters® PDA with SQD or Aglient® DAD with 6150 SQD instrument. The compounds according to Formulas I & II are prepared using conventional organic synthetic methods. A suitable synthetic route is depicted below in the following general reaction Schemes. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

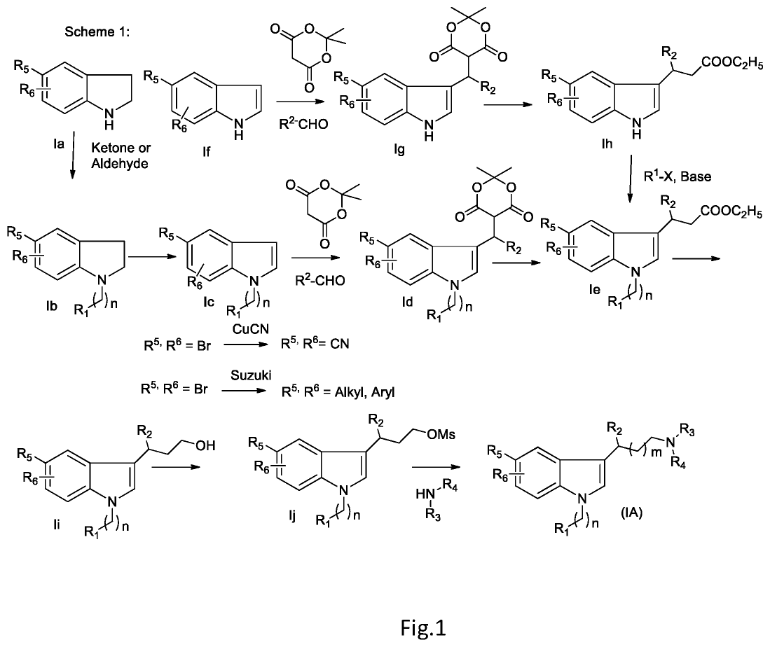

Scheme 1 (FIG. 1) shows a synthetic route for synthesis of compounds of general formula (IA) from compounds (Ia) or compounds (If). Reductive amination of (Ia) with appropeiate aldehyde or ketones of R₁ provide N-substituted indolonine derivatives (Ib) which upon oxidation give indole derivatives (Ic). Compounds of formula (Id) is obtained from compound of formula (Ic) via condensation reaction with R₂—CHO and Mandrolic ester, followed by reaction with Cu and ethyl alcohol gave compound of formula (Ie).

On the other hand compound of formula (Ie) can be obtain from Indole derivatives (If). Compound (Ig) is obtained from (If) by reaction with appropriate R₂CHO and Meldrum's acid and subsequent decarboxylation and esterification afford compound of formula (Ih). Key intermediate (Ie) is obtained alkylation of (Ih) with appropriate R1X. Compound (Ie) was reduced using procedure for the reduction ester known in literature to obtain compound (Ii), which on treatment with alkyl or aryl sulfonyl chloride or halogenating agent provide compound of formula (Ij). Finally, compound of formula IA is obtained by the reaction of compound Ij with appropriate amine (R₃R₄NH). In case, compound of formula Ic, where R₅, R₆ is halogen can be converted to R₅, R₆ is CN using cyanation reaction known in literature by CuCN. On the other hand, halogen is converted to aryl, alkyl group under Suzuki coupling known in literature. R₁ to R₆ containing N/O protecting group usually deprotected as and when required for further steps or to obtain final compound.

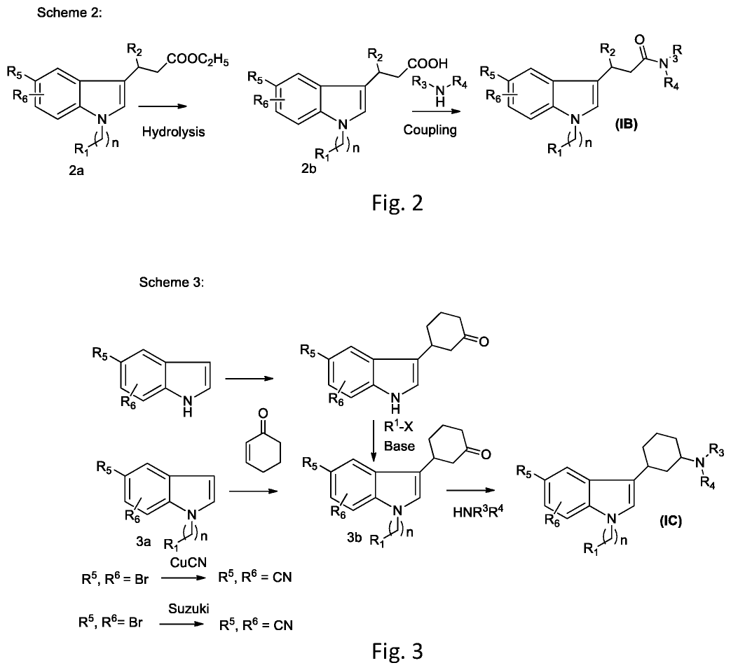

Scheme 2 (FIG. 2) shows synthetic route for synthesis of compounds of formula (B) from Compound 2a. Ester hydrolysis of 2a under basic condition known in literature afford compound 2b. Compound of formula 2b reacted with corresponding amine NHR₃R₄ as define above to get (IB). The reaction can be carried out using condition generally used for the synthesis of amide from acids under suitable coupling reagent or treating with halogenating reagents or dehydrating agent.

Scheme 3 (FIG. 3) shows a method of preparation of the compounds of formula (IC). Compound 3a can be prepared from 3a reacting with unsaturated ketone under Michael reaction condition in presence of Lewis acid. Compound 3b is treated with corresponding amine NHR₃R₄ under reductive amination condition know in literature to give compound of formula (IC).

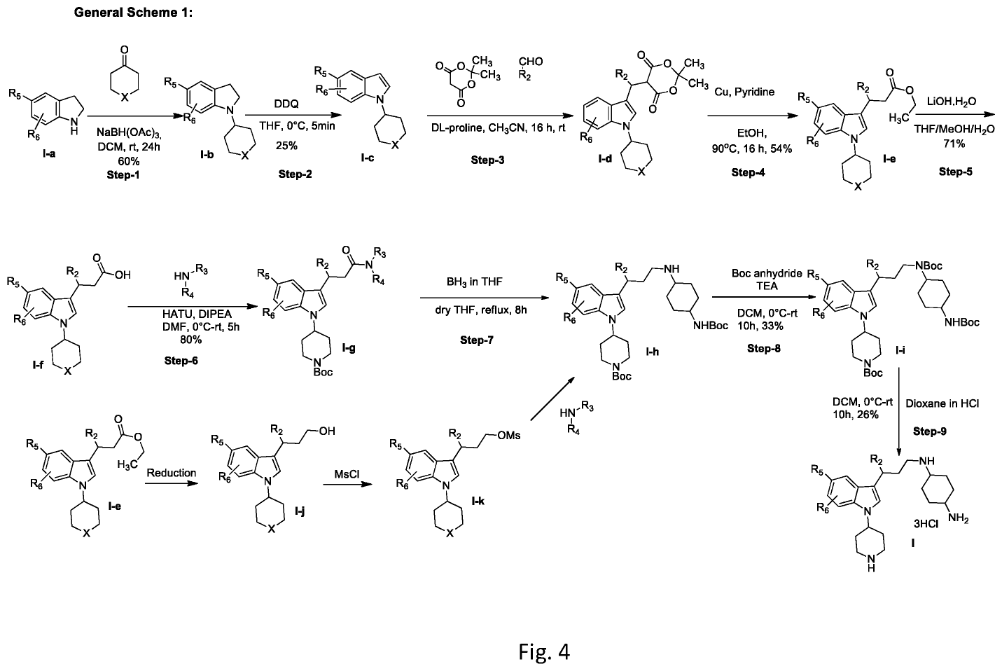

General scheme 1 (FIG. 4) describes synthesis of compound of formula F-I and I. Reductive amination of indoline derivative I-a with ketone provides I-b, which under oxidation by DDQ yields N-substituted indole compound I-c. 3-Substituted indole derivative I-d was obtained from Ic when treated with corresponding aldehyde R₂—CHO and Meldrum's acid followed by decarboxylation under Cu-EtOH give ester I-e. Saponification of I-e by LiOH, followed by coupling with proper NHR3R4 yielded compound I-g. Under amide reduction of I-g gave amine derivative I-h which was isolated as nonopolar Boc derivative by treatment with Boc anhydride. Finally compound I isolated as hydrochloride salt by deprotection of I-h under acidic condition. On the other hand, ester compound I-e was reduced to alcohol under reducing agent like LiAlH4 to obtain corresponding alcohol I-j, which on treatment with mesyl chloride to give mesyl derivative I-k, followed by displacement reaction with appropriate amine NHR³R⁴ gave compound of formula I-g. If R₃ and R⁴ contain N and O protecting group, which can be deprotected under various condition reported in literature to obtain final compound of formula F-I and I listed in Table 1.

### Example I: Synthesis N1-(3-(1-(piperidin-4-yl)-1H-indol-3-yl)-3-(m-tolyl) propyl) cyclohexane-1,4-diamine

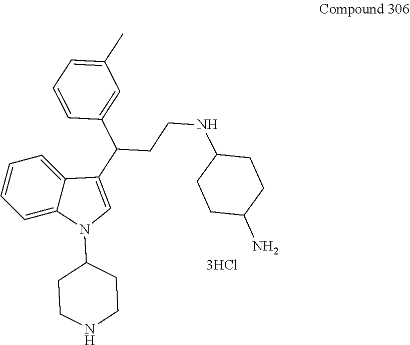 [SMILES: Cc1cccc(C(CCNC2CCC(N)CC2)c2cn(C3CCNCC3)c3ccccc23)c1]

### Synthesis of Tert-Butyl 4-(indolin-1-yl) piperidine-1-carboxylate

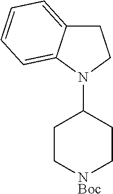 [SMILES: CC(C)(C)OC(=O)N1CCC(N2CCc3ccccc32)CC1]

To a stirred solution of Indoline (1 g, 8.403 mmol) in DCM (25 mL) was added tert-butyl 4-oxopiperidine-1-carboxylate (4.18 g, 21.008 mmol) and reaction mixture was stirred at rt, after 1 h of stirring, was added NaBH(OAC)₃ (2.67 g, 12.60 mmol) at 0° C. then stirred the reaction mixture at rt for 24 h. Progress of the reaction was monitored by TLC. The reaction mixture was diluted with aq NaHCO₃ solution (30 mL) and compound was extracted with DCM (3×50 mL). The organic layer was dried over anhydrous sodium sulphate, concentrated under reduced pressure. The crude compound was directly used in the next step without further purification (crude wt 1.8 g).

LC-MS m/z (M): calculated 302; found (M+H): 303

### Synthesis of Tert-Butyl 4-(1H-indol-1-yl) piperidine-1-carboxylate

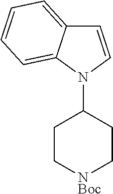 [SMILES: CC(C)(C)OC(=O)N1CCC(n2ccc3ccccc32)CC1]

To a stirred solution of tert-butyl 4-(indolin-1-yl) piperidine-1-carboxylate (2 g, 6.622 mmol) in THE (20 mL), was added DDQ (2.2 g, 9.933 mmol) at 0° C. and the reaction mixture was stirred at rt for 1 h. Progress of the reaction was monitored by TLC. Reaction mixture was diluted with water (50 mL), extracted with Ethyl acetate (3×60 mL). The organic layer was dried over anhydrous sodium sulphate, concentrated under reduced pressure. The crude compound was purified by column chromatography using 4% EtOAc in Pet-ether as an eluent to afford desired product as gummy mass (yield: 250 mg, 25%).

¹H NMR (400 MHz, CDCl₃) δ 7.65 (d, J=4.9 Hz, 1H), 7.39 (d, J=9.49 Hz, 2H), 7.23-7.15 (m, 2H), 7.10 (t, J=7.14 Hz, 1H), 6.54 (d, J=10.7 Hz, 1H), 4.40-4.28 (m, 2H), 2.92 (t, J=12.08 Hz, 2H), 2.12-2.05 (m, 2H), 1.94-1.85 (m, 2H), 1.5 (s, 10H)

### Synthesis of Tert-Butyl 4-(3-((2,2-dimethyl-4,6-dioxo-1,3-dioxan-5-yl)(m-tolyl)methyl)-1H-indol-1-yl)piperidine-1-carboxylate

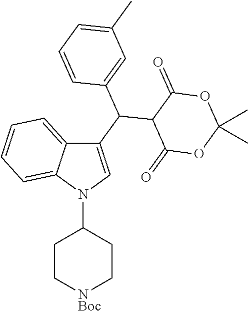 [SMILES: Cc1cccc(C(c2cn(C3CCN(C(=O)OC(C)(C)C)CC3)c3ccccc23)C2C(=O)OC(C)(C)OC2=O)c1]

To a stirred solution of tert-butyl 4-(1H-indol-1-yl) piperidine-1-carboxylate (520 mg, 1.73 mmol) in dry Acetonitrile (6 mL), were added Meldrum's acid (499 mg, 3.46 mmol), m-tolualdehyde (270 mg, 2.25 mmol) and L-proline (20 mg, 0.173 mmol) then reaction mixture was stirred at rt for 16 h. Progress of the reaction was monitored by TLC. The reaction mixture was concentrated under vacuum and the crude product was carried forward to next step without purification (crude wt: 1.3 g).

LC-MS m/z (M): calculated 546.6

### Synthesis of Ethyl Ethyl 3-(1-(piperidin-4-yl)-1H-indol-3-yl)-3-(m-tolyl) Propanoate

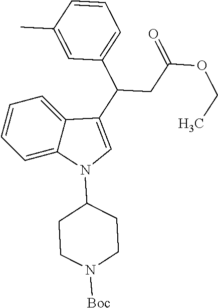 [SMILES: CCOC(=O)CC(c1cccc(C)c1)c1cn(C2CCN(C(=O)OC(C)(C)C)CC2)c2ccccc12]

To a stirred solution of tert-butyl 4-(3-((2,2-dimethyl-4,6-dioxo-1,3-dioxan-5-yl) (m-tolyl)methyl)-1H-indol-1-yl)piperidine--carboxylate (1.3 g, 2.380 mmol) in a 1:1 mixture of pyridine and Ethanol (20 mL) was added Cu powder (15 mg, 0.238 mmol) and stirred the reaction mixture at 90° C. for 16 h. The progress of the reaction was monitored by TLC. The reaction mixture was filtered and the filtrate was concentrated under reduced pressure. The crude compound was purified by column chromatography (silica gel 60-120 mesh, eluted with 10% EtOAc in Pet-ether) to afford desired product as yellow liquid (yield: 600 mg, 54%).

### Synthesis of 3-(1-(1-(tert-butoxycarbonyl) piperidin-4-yl)-1H-indol-3-yl)-3-(m-tolyl)propanoic Acid

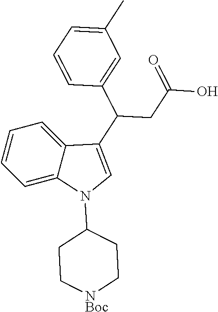 [SMILES: Cc1cccc(C(CC(=O)O)c2cn(C3CCN(C(=O)OC(C)(C)C)CC3)c3ccccc23)c1]

To a stirred solution of 3-(1-(1-(tert-butoxycarbonyl) piperidin-4-yl)-1H-indol-3-yl)-3-(m-tolyl)propanoic acid (530 mg, 1.08 mmol) in THF/MeOH/H₂O (1:1:1) (15 mL) was added LiOH (454 mg, 10.8 mmol) at 0° C. and the reaction mixture was stirred at rt for 6 h. Progress of the reaction was monitored by TLC. The reaction mixture was acidified to pH 6 with citric acid. Off white solid was thrown out during acidification was filtered and air dried (yield: 358 mg, 71%). ¹H NMR (400 MHz, DMSO-d6) δ 7.44 (d, J=7.8 Hz, 1H), 7.31 (d, J=8.38 Hz, 1H), 7.20-7.08 (m, 4H), 7.09-6.98 (m, 3H), 4.74 (t, J=7.87 Hz, 1H), 4.38-4.25 (m, 3H), 3.20-3.12 (m, 1H), 3.09-3.02 (m, 1H), 2.90-2.87 (m, 2H), 2.29 (s, 3H), 2.10-2.0 (m, 2H), 1.92-1.84 (m, 2H), 1.49 (s, 9H) LC-MS m/z (M): calculated 462.59; found (M−H): 461.2

### Synthesis of Tert-Butyl 4-(3-(3-((4-((tert-butoxycarbonyl) amino)cyclohexyl)amino)-3-oxo-1-(m-tolyl)propyl)-1H-indol-1-yl)piperidine-1-carboxylate

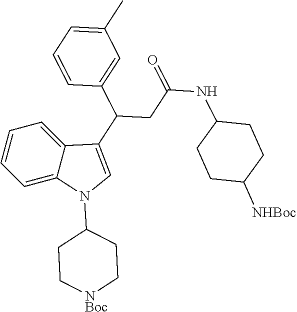 [SMILES: Cc1cccc(C(CC(=O)NC2CCC(NC(=O)OC(C)(C)C)CC2)c2cn(C3CCN(C(=O)OC(C)(C)C)CC3)c3ccccc23)c1]

To a stirred solution of 3-(1-(1-(tert-butoxycarbonyl) piperidin-4-yl)-1H-indol-3-yl)-3-(m-tolyl)propanoic acid (350 mg, 0.756 mmol) in DMF (2 mL), were added DIPEA (0.270 mL, 1.512 mmol), HATU (430 mg, 1.134 mmol) followed by tert-butyl (4-aminocyclohexyl)carbamate (210 mg, 0.983 mmol) at 0° C. and the reaction mixture was stirred at rt for 5 h. Progress of the reaction was monitored by TLC. Ice cold water added to reaction mixture at 0° C., extracted with EtOAc. The combined organic layer dried over Na₂SO₄ and concentrated under reduced pressure. The crude compound was purified by column chromatography, eluted with 20% EtOAc in Pet-ether to afford desired product as off white solid (yield: 400 mg, 80%).

¹H NMR (400 MHz, DMSO-d6) δ 7.58 (d, J=7.92 Hz, 1H), 7.36 (d, J=8.22 Hz, 1H), 7.26-7.20 (m, 1H), 7.18-7.08 (m, 5H), 7.0 (d, J=6.54 Hz, 1H), 5.28-5.25 (m, 1H), 4.63 (t, J=7.53 Hz, 1H), 4.39-4.31 (m, 3H), 3.85-3.62 (m, 3H), 3.35-3.2 (m, 1H), 3.19-3.0 (m, 8H), 2.30 (s, 3H), 2.11-2.0 (m, 2H), 1.91-1.83 (m, 2H), 1.75-1.70 (m, 2H), 1.57-1.51 (m, 20H), 1.40-1.20 (m, 5H)

LC-MS m/z (M): calculated 658.87; found (M+H): 659.4

### Synthesis of Tert-Butyl 4-(3-(3-((4-((tert-butoxycarbonyl)amino)cyclohexyl)amino)-1-(m-tolyl)propyl)-1H-indol-1-yl)piperidine-1-carboxylate

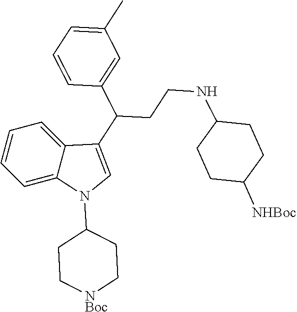 [SMILES: Cc1cccc(C(CCNC2CCC(NC(=O)OC(C)(C)C)CC2)c2cn(C3CCN(C(=O)OC(C)(C)C)CC3)c3ccccc23)c1]

To a stirred solution of tert-butyl 4-(3-(3-((4-((tert-butoxycarbonyl) amino)cyclohexyl)amino)-1-(m-tolyl)propyl)-1H-indol-1-yl)piperidine-1-carboxylate (200 mg, 0.303 mmol) in dry THF (8 mL), was added BH₃ in THF (1M, 4.5 mL, 4.553 mmol) at 0° C. and the reaction mixture was refluxed for 8 h. Progress of the reaction was monitored by TLC. After 8 h of reflux, 5 mL of MeOH was added then refluxed for 5 h. Solvent was removed from reaction mixture under reduced pressure and the crude compound was directly carry forwarded to the next step without further purification (crude yield 220 mg).

### Synthesis of Tert-Butyl 4-(3-(3-((tert-butoxycarbonyl) (4-((tert-butoxycarbonyl)amino) cyclohexyl)amino)-1-(m-tolyl)propyl)-1H-indol-1-yl) piperidine-1-carboxylate

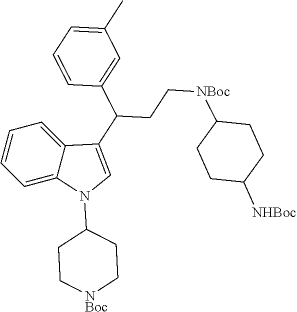 [SMILES: Cc1cccc(C(CCN(C(=O)OC(C)(C)C)C2CCC(NC(=O)OC(C)(C)C)CC2)c2cn(C3CCN(C(=O)OC(C)(C)C)CC3)c3ccccc23)c1]

To a stirred solution of tert-butyl 4-(3-(3-((4-((tert-butoxycarbonyl) amino)cyclohexyl)amino)-1-(m-tolyl)propyl)-1H-indol-1-yl)piperidine-1-carboxylate (220 mg, 0.34 mmol), in DCM (5 mL) were added TEA (0.25 mL, 1.7 mmol), followed by Boc anhydride (0.37 mL, 1.7 mmol), and the reaction mixture was stirred at rt for 12 h. Progress of the reaction was monitored by TLC. Excess solvent was removed from the reaction mixture and the crude compound was purified by column chromatography using 25% EtOAc in Hexane as an eluent to afford desired compound as colorless liquid (yield: 65 mg, 25%).

### Synthesis of N1-(3-(1-(piperidin-4-yl)-1H-indol-3-yl)-3-(m-tolyl) propyl)cyclohexane-1,4-diamine Trihydrochloride

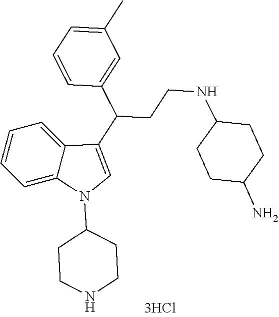 [SMILES: Cc1cccc(C(CCNC2CCC(N)CC2)c2cn(C3CCNCC3)c3ccccc23)c1]

To a stirred solution of tert-butyl 4-(3-(3-((tert-butoxycarbonyl) (4-((tert-butoxycarbonyl)amino)cyclohexyl)amino)-1-(m-tolyl)propyl)-1H-indol-1-yl)piperidine-1-carboxylate (65 mg, 0.087) in DCM (2 mL), was added HCl in dioxane (4M, 1.2 mL) at 0° C. and reaction mixture was stirred at rt for 2 h. Progress of the reaction was monitored by TLC. Excess solvent was removed under reduced pressure and washed with diethyl ether to get an off white solid (yield: 10 mg, 26%).

¹H NMR (400 MHz, DMSO-d6) δ 8.90-8.85 (m, 3H), 8.7 (brs, 1H), 7.96 (brs, 1H), 7.53 (d, J=8.1 Hz, 1H), 7.45 (d, J=8.04 Hz, 1H), 7.33 (s, 1H), 7.10-7.19 (m, 4H), 6.99-6.94 (m, 2H), 4.70-4.65 (m, 1H), 4.28-4.25 (m, 1H), 3.43 (d, J=11 Hz, 2H), 3.30-3.12 (m, 5H), 2.95-2.90 (m, 1H), 2.80-2.72 (m, 1H), 2.40-2.35 (m, 1H), 2.25-2.18 (m, 5H), 2.17-2.12 (m, 2H), 1.95-1.90 (m, 1H), 1.80-1.62 (m, 8H),

LC-MS m/z (M): calculated 445.6; found (M+H): 446.4

### Synthesis of 3-(3-((3-aminopropyl) amino)-1-(3-(trifluoromethoxy)phenyl)propyl)-1-cyclohexyl-1H-indole-5-carbonitrile Dihydrochloride

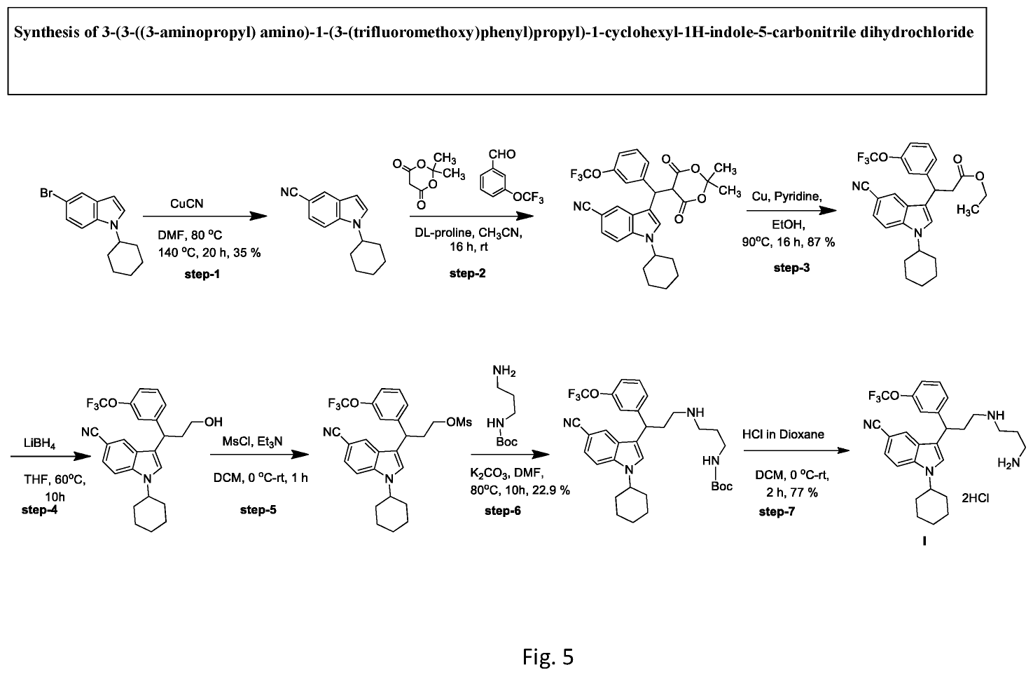

See FIG. 5.

### Synthesis of 1-cyclohexyl-1H-indole-5-carbonitrile

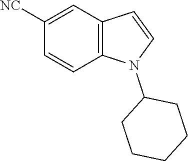 [SMILES: N#Cc1ccc2c(ccn2C2CCCCC2)c1]

To a stirred solution of 5-bromo-1-cyclohexyl-1H-indole (3 g, 11.07 mmol) in DMF, was added CuCN (2.95 g, 33.21 mmol) and the reaction mixture was stirred at 140° C. for 20 h. Progress of the reaction was monitored by TLC. Reaction mixture was diluted with ice cold water (50 mL), extracted with Ethyl acetate (3×50 mL). The organic layer was dried over anhydrous sodium sulphate, concentrated under reduced pressure. The crude compound was purified by column chromatography using 5% EtOAc in Pet-ether as an eluent to afford desired product as colourless viscous liquid (yield: 850 mg, 35%).

¹H NMR (400 MHz, CDCl₃) δ 7.9 (s, 1H), 7.41 (s, 2H), 7.34 (d, J=3.29 Hz, 1H), 6.58 (d, J=3.25 Hz, 1H), 4.28-4.19 (m, 1H), 2.12 (d, J=11.58 Hz, 2H), 1.96 (d, J=13.47 Hz, 2H), 1.80-1.85 (m, 1H), 1.78-1.62 (m, 2H), 1.53-1.48 (m, 2H), 1.45-1.23 (m, 1H)

LC-MS m/z (M): calculated 224.3; found (M+H): 225.2

### Synthesis of 1-cyclohexyl-3-((2,2-dimethyl-4,6-dioxo-1,3-dioxan-5-yl) (3-(trifluoromethoxy) phenyl)methyl)-1H-indole-5-carbonitrile

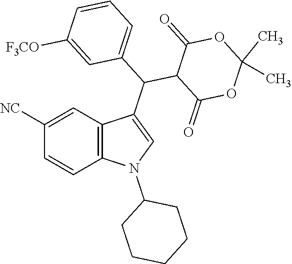 [SMILES: CC1(C)OC(=O)C(C(c2cccc(OC(F)(F)F)c2)c2cn(C3CCCCC3)c3ccc(C#N)cc23)C(=O)O1]

To a stirred solution of 1-cyclohexyl-1H-indole-5-carbonitrile (830 mg, 3.700 mmol) in dry Acetonitrile, were added Meldrum's acid (959 mg, 6.66 mmol), 3-(trifluoromethoxy) benzaldehyde (0.68 mL, 4.81 mmol) and DL-proline (43 mg, 0.37 mmol) then reaction mixture was stirred at rt for 16 h. Progress of the reaction was monitored by TLC. The reaction mixture was concentrated under vacuum and the crude product was carried forward to next step without purification (crude wt 3.26 g).

LC-MS m/z (M): calculated 540.5; found (M+H): 541.18

### Synthesis of Ethyl 3-(5-cyano-1-cyclohexyl-1H-indol-3-yl)-3-(3-(trifluoromethoxy) phenyl)propanoate

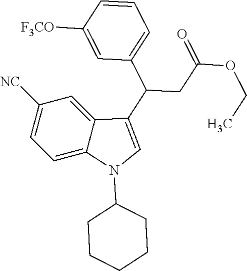 [SMILES: CCOC(=O)CC(c1cccc(OC(F)(F)F)c1)c1cn(C2CCCCC2)c2ccc(C#N)cc12]

To a stirred solution of 1-cyclohexyl-3-((2,2-dimethyl-4,6-dioxo-1,3-dioxan-5-yl) (3-(trifluoromethoxy)phenyl)methyl)-1H-indole-5-carbonitrile (3.26 g, 6.03 mmol) in a 1:1 mixture of pyridine and Ethanol (40 mL) was added Cu powder (77 mg, 1.206 mmol) and stirred the reaction mixture at 90° C. for 16 h. Progress of the reaction was monitored by TLC. The reaction mixture was filtered and the filtrate was concentrated under reduced pressure. The crude compound was purified by column chromatography (silica gel 60-120 mesh, eluted with 10% EtOAc in Pet-ether) to afford desired product as yellow solid (yield: 1.57 g, 87%).

¹H NMR (400 MHz, CDCl₃) δ 7.67 (s, 1H), 7.37 (s, 2H), 7.36-7.30 (m, 1H), 7.25-7.20 (m, 2H), 7.10-7.08 (m, 2H), 4.78 (t, J=7.91 Hz, 1H), 4.22-4.16 (m, 1H), 4.08-4.0 (m, 2H), 3.12-3.05 (m, 1H), 3.04-2.95 (m, 1H), 2.15-2.02 (m, 3H), 2.0-1.92 (m, 2H), 1.85-1.79 (m, 1H), 1.76-1.62 (m, 2H), 1.52-1.46 (m, 2H), 1.35-1.24 (m, 2H), 1.19-1.10 (m, 3H)

LC-MS m/z (M): calculated 484.5; found (M+H): 485.2

### Synthesis of 1-cyclohexyl-3-(3-hydroxy-1-(3-(trifluoromethoxy) phenyl)propyl)-1H-indole-5-carbonitrile

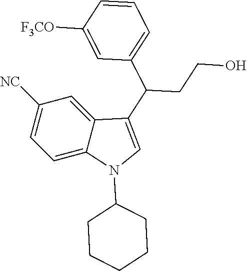 [SMILES: N#Cc1ccc2c(c1)c(C(CCO)c1cccc(OC(F)(F)F)c1)cn2C1CCCCC1]

To a stirred solution of ethyl 3-(5-cyano-1-cyclohexyl-1H-indol-3-yl)-3-(3-(trifluoromethoxy) phenyl)propanoate (1.55 g, 3.199) in dry THF, was added LiBH₄ (211 mg, 9.597 mmol) at 0° C. and reaction mixture was stirred at 60° C. for 10 h. Progress of the reaction was monitored by TLC. Reaction mixture was quenched with ice cold water, extracted with DCM. Combined organic layer was dried over Na₂SO₄ and concentrated under reduced pressure. The crude product was carried forward to next step without purification (crude wt: 1.5 g).

¹H NMR (400 MHz, CDCl₃) δ 7.97 (s, 1H), 7.75 (s, 1H), 7.67 (d, J=8.65 Hz, 1H), 7.42-7.38 (m, 4H), 7.15-7.10 (m, 1H), 4.50-4.20 (m, 4H), 3.38-3.36 (m, 2H), 2.32-2.26 (m, 1H), 2.20-2.10 (m, 1H), 1.98-1.88 (m, 2H), 1.87-1.60 (m, 6H), 1.58-1.40 (m, 3H), 1.30-1.20 (m, 2H), 1.18-1.12 (m, 1H)

LC-MS m/z (M): calculated 442.4; found (M+H): 443.2

### Synthesis of 3-(5-cyano-1-cyclohexyl-1H-indol-3-yl)-3-(3-(trifluoromethoxy) phenyl)propyl Methanesulfonate

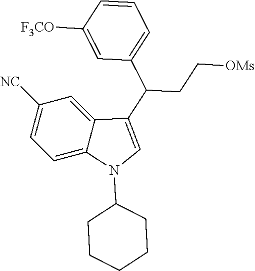 [SMILES: CS(=O)(=O)OCCC(c1cccc(OC(F)(F)F)c1)c1cn(C2CCCCC2)c2ccc(C#N)cc12]

To a stirred solution of 1-cyclohexyl-3-(3-hydroxy-1-(3-(trifluoromethoxy) phenyl)propyl)-1H-indole-5-carbonitrile (520 mg, 1.176 mmol) in CH₂Cl₂ (6 mL), were added TEA (0.33 mL, 2.352 mmol) followed by methane sulphonylchloride (0.11 mL, 1.411 mmol) dropwise at 0° C. and stirred the reaction mixture at room temperature for 2 h. Progress of the reaction was monitored by TLC. The reaction mixture was diluted with H₂O (20 mL) and compound was extracted with CH₂Cl₂ (3×20 mL), combined organic layer was washed with saturated NaHCO₃ (20 mL), which was dried over anhydrous sodium sulphate, concentrated under reduced pressure. The crude compound was carried forward to next step without purification (crude wt: 630 mg).

LC-MS m/z (M): calculated 520.5; found (M+H): 521.2

### Synthesis of Tert-Butyl (3-((3-(5-cyano-1-cyclohexyl-1H-indol-3-yl)-3-(3-(trifluoromethoxy)phenyl)propyl)amino)propyl)carbamate

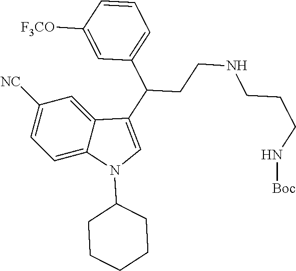 [SMILES: CC(C)(C)OC(=O)NCCCNCCC(c1cccc(OC(F)(F)F)c1)c1cn(C2CCCCC2)c2ccc(C#N)cc12]

To a stirred solution of 3-(5-cyano-1-cyclohexyl-1H-indol-3-yl)-3-(3-(trifluoromethoxy) phenyl)propyl methanesulfonate (630 mg, 1.210 mmol) in dry DMF (5 mL), were added K₂CO3 (500 mg, 3.63 mmol) and tert-butyl (3-aminopropyl)carbamate (253 mg, 1.452 mmol) then the reaction mixture was stirred at 80° C. for 10 h. Progress of the reaction was monitored by TLC. The reaction mixture was poured in to ice-cold water (20 mL), solid was precipitated out, which was filtered and soluble in CH₂Cl₂ (20 mL), concentrated under reduced pressure. The crude compound was purified by preparative TLC (eluted with 5% MeOH in CH₂Cl₂) to afford desired product as light brown liquid (yield: 166 mg, 22.9%).

¹H NMR (400 MHz, DMSO-d6) δ 7.94 (s, 1H), 7.75 (s, 1H), 7.68 (d, J=8.65 Hz, 1H), 7.42-7.35 (m, 4H), 7.15-7.10 (m, 1H), 6.82-6.79 (m, 1H), 4.42-4.35 (m, 2H), 4.10-4.05 (m, 2H), 3.18-3.13 (m, 5H), 2.96-2.90 (m, 2H), 2.46-2.40 (m, 3H), 2.30-2.22 (m, 1H), 2.20-2.12 (m, 1H), 1.96-1.88 (m, 2H), 1.86-1.78 (m, 4H), 1.76-1.68 (m, 1H), 1.56-1.48 (m, 4H), 1.34 (s, 9H), 1.25-1.20 (m, 3H)

LC-MS m/z (M): calculated 598.2; found (M+H): 599.45

### Synthesis of 3-(3-((3-aminopropyl) amino)-1-(3-(trifluoromethoxy)phenyl)propyl)-1-cyclohexyl-1H-indole-5-carbonitrile Dihydrochloride

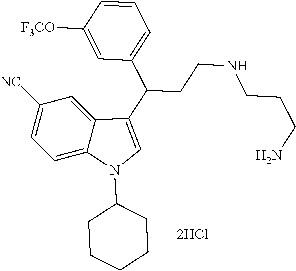 [SMILES: N#Cc1ccc2c(c1)c(C(CCNCCCN)c1cccc(OC(F)(F)F)c1)cn2C1CCCCC1]

To a stirred solution of tert-butyl (3-((3-(5-cyano-1-cyclohexyl-1H-indol-3-yl)-3-(3-(trifluoromethoxy)phenyl)propyl)amino)propyl)carbamate (160 mg, 0.267 mmol) in DCM (2 mL), was added HCl in dioxane(4M, 2 mL) at 0° C. and the reaction mixture was stirred at room temperature for 2 h. The reaction mixture was concentrated under reduced pressure and the crude compound was washed with diethyl ether to afford desired compound as off white solid (yield: 118 mg, 77%). MP: 190-194° C.

¹H NMR (400 MHz, DMSO-d6) δ 9.38-9.30 (m, 2H), 8.00-7.70 (m, 5H), 7.71 (d, J=8.61 Hz, 1H), 7.44-7.42 (m, 4H), 7.18 (brs, 1H), 4.55 (t, J=7.40 Hz, 1H), 4.42-4.39 (m, 1H), 3.10-2.77 (m, 6H), 2.60-2.55 (m, 1H), 2.43-2.38 (m, 1H), 1.95-1.93 (m, 4H), 1.86-1.81 (m, 4H), 1.77-1.73 (m, 1H), 1.59-1.42 (m, 2H), 1.35-1.20 (m, 2H)

LC-MS m/z (M): calculated 498.5; found (M+H): 499.3

### Synthesis of 3-(3-((3-aminopropyl) amino)-1-(3-(trifluoromethoxy)phenyl)propyl)-1-cyclohexyl-1H-indole-5-carboxamide

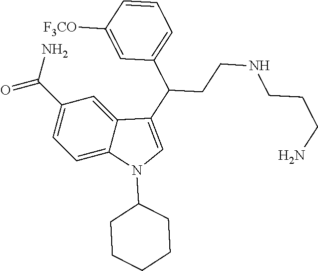 [SMILES: NCCCNCCC(c1cccc(OC(F)(F)F)c1)c1cn(C2CCCCC2)c2ccc(C(N)=O)cc12]

To a stirred solution of tert-butyl (3-((3-(5-cyano-1-cyclohexyl-1H-indol-3-yl)-3-(3-(trifluoromethoxy) phenyl)propyl)amino)propyl)carbamate (30 mg, 0.058 mmol) in EtOH:H₂O (9:1) (2 mL), was added KOH and the reaction mixture was stirred at 90° C. for 50 h. Progress of the reaction was monitored by TLC. Reaction mixture was cooled to rt, acidified with 6N HCl until pH of the reaction mixture became 1 and compound extracted with 10% MeOH in DCM. Organic layer was dried over sodium sulphate and concentrated afford desired compound as off white solid (yield: 6 mg, 25%).

¹H NMR (400 MHz, DMSO-d6) δ 8.10 (brs, 1H), 7.80 (brs, 1H), 7.68-7.61 (m, 2H), 7.51 (d, J=8.69 Hz, 1H), 7.40-7.34 (m, 3H), 7.13-7.07 (m, 2H), 4.34 (t, J=11.72 Hz, 1H), 2.85-2.81 (m, 2H), 2.74-2.70 (m, 2H), 2.29-2.25 (m, 2H), 2.00-1.93 (m, 2H), 1.89 (s, 1H), 1.87-1.72 (m, 7H), 1.54-1.45 (m, 2H), 1.32-1.22 (m, 4H)

LC-MS m/z (M): calculated 516.6; found (M+H): 517.2

Following the procedure described in scheme 1/Example A, compounds of Table 1 are prepared by using suitable starting materials and proper conditions.

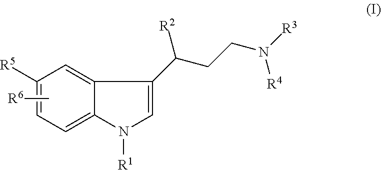

TABLE 1

| Cmpd | R¹ | R² | R³ | R⁴ | R⁵ | R⁶ |
|---|---|---|---|---|---|---|
| 306 | 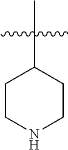 [SMILES: *C1CCNCC1] | 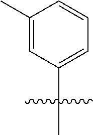 [SMILES: *c1cccc(C)c1] | H | 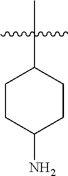 [SMILES: *C1CCC(N)CC1] | H | H |
| 307 | 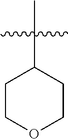 [SMILES: *C1CCOCC1] | 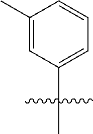 [SMILES: *c1cccc(C)c1] | H | 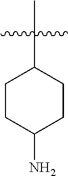 [SMILES: *C1CCC(N)CC1] | H | H |
| 308 | 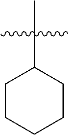 [SMILES: *C1CCCCC1] | 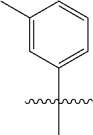 [SMILES: *c1cccc(C)c1] | H | 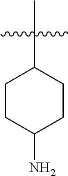 [SMILES: *C1CCC(N)CC1] | H | H |
| 309 | 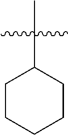 [SMILES: *C1CCCCC1] | 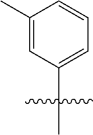 [SMILES: *c1cccc(C)c1] | H | 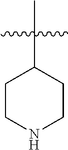 [SMILES: *C1CCNCC1] | H | H |
| 310 | 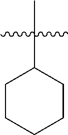 [SMILES: *C1CCCCC1] | 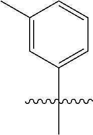 [SMILES: *c1cccc(C)c1] | H | 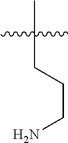 [SMILES: *CCCN] | H | H |
| 317 | 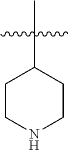 [SMILES: *C1CCNCC1] | 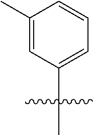 [SMILES: *c1cccc(C)c1] | H | 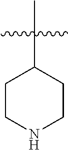 [SMILES: *C1CCNCC1] | H | H |
| 322 | 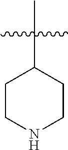 [SMILES: *C1CCNCC1] | 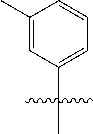 [SMILES: *c1cccc(C)c1] | H | 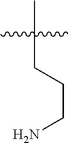 [SMILES: *CCCN] | H | H |
| 332 | 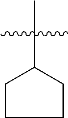 [SMILES: *C1CCCC1] | 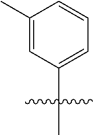 [SMILES: *c1cccc(C)c1] | H | 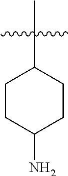 [SMILES: *C1CCC(N)CC1] | H | H |
| 333 | 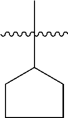 [SMILES: *C1CCCC1] | 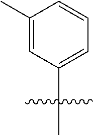 [SMILES: *c1cccc(C)c1] | H | 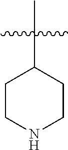 [SMILES: *C1CCNCC1] | H | H |
| 334 | 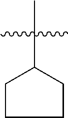 [SMILES: *C1CCCC1] | 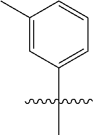 [SMILES: *c1cccc(C)c1] | H | 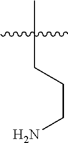 [SMILES: *CCCN] | H | H |
| 323 | 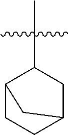 [SMILES: *C1CC2CCC1C2] | 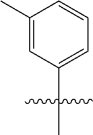 [SMILES: *c1cccc(C)c1] | H | 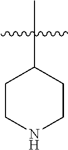 [SMILES: *C1CCNCC1] | H | H |
| 324 | 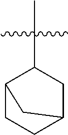 [SMILES: *C1CC2CCC1C2] | 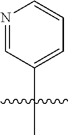 [SMILES: *c1cccnc1] | H | 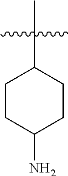 [SMILES: *C1CCC(N)CC1] | H | H |
| 325 | 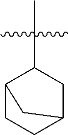 [SMILES: *C1CC2CCC1C2] | 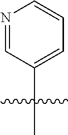 [SMILES: *c1cccnc1] | H | 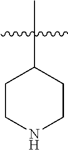 [SMILES: *C1CCNCC1] | H | H |
| 328 | 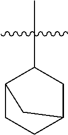 [SMILES: *C1CC2CCC1C2] | 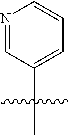 [SMILES: *c1cccnc1] | H | 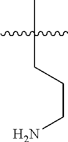 [SMILES: *CCCN] | H | H |
| 329 | 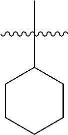 [SMILES: *C1CCCCC1] | 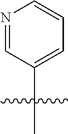 [SMILES: *c1cccnc1] | H | 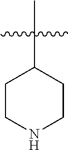 [SMILES: *C1CCNCC1] | H | H |
| 330 | 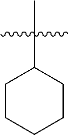 [SMILES: *C1CCCCC1] | 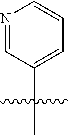 [SMILES: *c1cccnc1] | H | 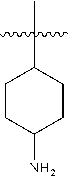 [SMILES: *C1CCC(N)CC1] | H | H |
| 331 | 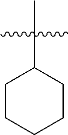 [SMILES: *C1CCCCC1] | 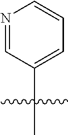 [SMILES: *c1cccnc1] | H | 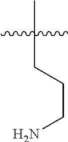 [SMILES: *CCCN] | H | H |
| 342 | 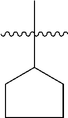 [SMILES: *C1CCCC1] | 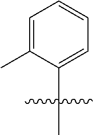 [SMILES: *c1ccccc1C] | H | 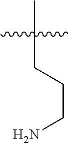 [SMILES: *CCCN] | H | H |
| 343 | 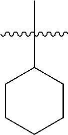 [SMILES: *C1CCCCC1] | 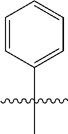 [SMILES: *c1ccccc1] | H | 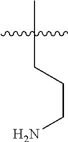 [SMILES: *CCCN] | H | H |
| 344 | 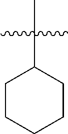 [SMILES: *C1CCCCC1] | 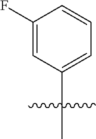 [SMILES: *c1cccc(F)c1] | H | 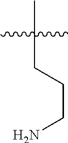 [SMILES: *CCCN] | H | H |
| 335 | 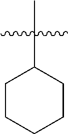 [SMILES: *C1CCCCC1] | 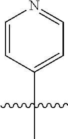 [SMILES: *c1ccncc1] | H | 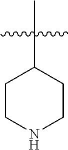 [SMILES: *C1CCNCC1] | H | H |
| 336 | 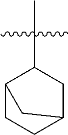 [SMILES: *C1CC2CCC1C2] | 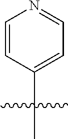 [SMILES: *c1ccncc1] | H | 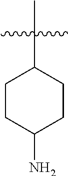 [SMILES: *C1CCC(N)CC1] | H | H |
| 337 | 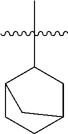 [SMILES: *C1CC2CCC1C2] | 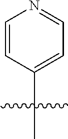 [SMILES: *c1ccncc1] | H | 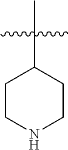 [SMILES: *C1CCNCC1] | H | H |
| 338 | 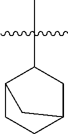 [SMILES: *C1CC2CCC1C2] | 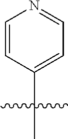 [SMILES: *c1ccncc1] | H | 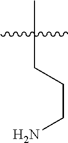 [SMILES: *CCCN] | H | H |
| 339 | 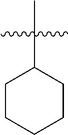 [SMILES: *C1CCCCC1] | 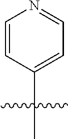 [SMILES: *c1ccncc1] | H | 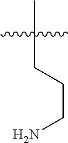 [SMILES: *CCCN] | H | H |
| 340 | 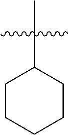 [SMILES: *C1CCCCC1] | 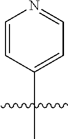 [SMILES: *c1ccncc1] | H | 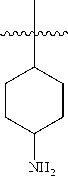 [SMILES: *C1CCC(N)CC1] | H | H |
| 341 | 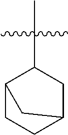 [SMILES: *C1CC2CCC1C2] | 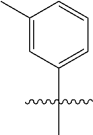 [SMILES: *c1cccc(C)c1] | H | 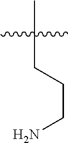 [SMILES: *CCCN] | H | H |
| 354 | 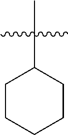 [SMILES: *C1CCCCC1] | 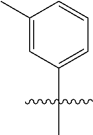 [SMILES: *c1cccc(C)c1] | CO Me | 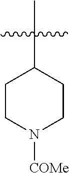 | H | H |
| 355 | 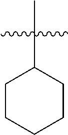 [SMILES: *C1CCCCC1] | 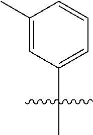 [SMILES: *c1cccc(C)c1] | H | 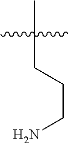 [SMILES: *CCCN] | Br | H |
| 356 | 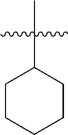 [SMILES: *C1CCCCC1] | 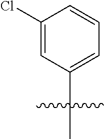 [SMILES: *c1cccc(Cl)c1] | H | 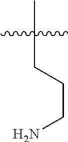 [SMILES: *CCCN] | H | H |
| 357 | 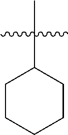 [SMILES: *C1CCCCC1] | 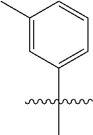 [SMILES: *c1cccc(C)c1] | H | 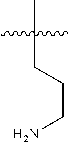 [SMILES: *CCCN] | H | Br |
| 358 | 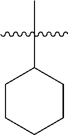 [SMILES: *C1CCCCC1] | 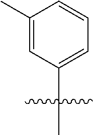 [SMILES: *c1cccc(C)c1] | H | 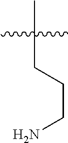 [SMILES: *CCCN] | H | H |
| 359 | 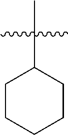 [SMILES: *C1CCCCC1] | H | H | 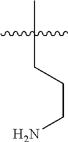 [SMILES: *CCCN] | Br | H |
| 345 | 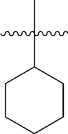 [SMILES: *C1CCCCC1] | 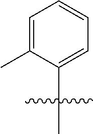 [SMILES: *c1ccccc1C] | H | 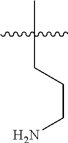 [SMILES: *CCCN] | H | H |
| 346 | 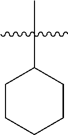 [SMILES: *C1CCCCC1] | 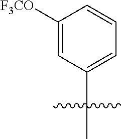 [SMILES: *c1cccc(OC(F)(F)F)c1] | H | 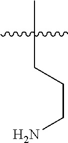 [SMILES: *CCCN] | H | H |
| 349 | 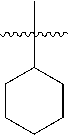 [SMILES: *C1CCCCC1] | 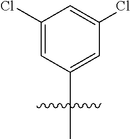 [SMILES: *c1cc(Cl)cc(Cl)c1] | H | 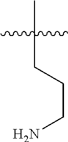 [SMILES: *CCCN] | H | H |
| 350 | 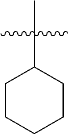 [SMILES: *C1CCCCC1] | 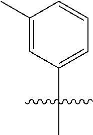 [SMILES: *c1cccc(C)c1] | H | 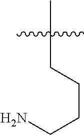 [SMILES: *CCCCN] | H | H |
| 351 | 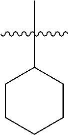 [SMILES: *C1CCCCC1] | 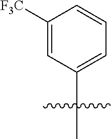 [SMILES: *c1cccc(C(F)(F)F)c1] | H | 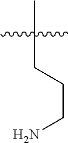 [SMILES: *CCCN] | H | H |
| 352 | 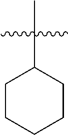 [SMILES: *C1CCCCC1] | 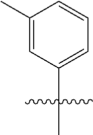 [SMILES: *c1cccc(C)c1] | H | H | H | H |
| 353 | 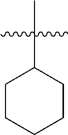 [SMILES: *C1CCCCC1] | 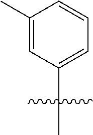 [SMILES: *c1cccc(C)c1] | H | 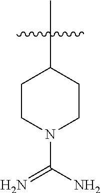 [SMILES: *C1CCN(C(=N)N)CC1] | | |
| 364 | 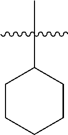 [SMILES: *C1CCCCC1] | 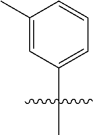 [SMILES: *c1cccc(C)c1] | H | 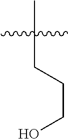 [SMILES: *CCCO] | H | H |
| 365 | 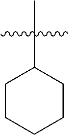 [SMILES: *C1CCCCC1] | 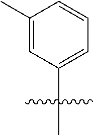 [SMILES: *c1cccc(C)c1] | H | 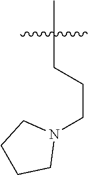 [SMILES: *CCCN1CCCC1] | H | H |
| 368 | 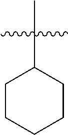 [SMILES: *C1CCCCC1] | 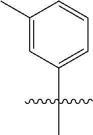 [SMILES: *c1cccc(C)c1] | H | 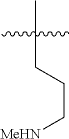 [SMILES: *CCCNC] | H | H |
| 369 | 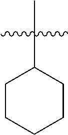 [SMILES: *C1CCCCC1] | 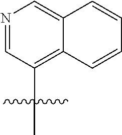 [SMILES: *c1cncc2ccccc12] | H | 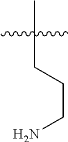 [SMILES: *CCCN] | H | H |
| 371 | 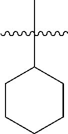 [SMILES: *C1CCCCC1] | 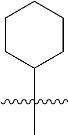 [SMILES: *C1CCCCC1] | H | 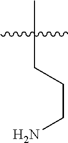 [SMILES: *CCCN] | H | H |
| 372 | 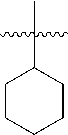 [SMILES: *C1CCCCC1] | 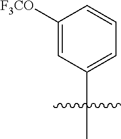 [SMILES: *c1cccc(OC(F)(F)F)c1] | H | 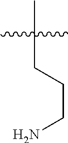 [SMILES: *CCCN] | CN | H |
| 360 | 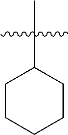 [SMILES: *C1CCCCC1] | H | H | 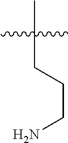 [SMILES: *CCCN] | 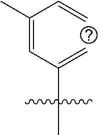 [SMILES: *C(=C)/C=C(/C)C=C] | H |
| 361 | 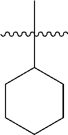 [SMILES: *C1CCCCC1] | 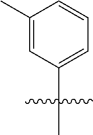 [SMILES: *c1cccc(C)c1] | CN | 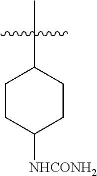 | H | H |
| 362 | 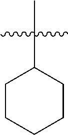 [SMILES: *C1CCCCC1] | 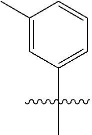 [SMILES: *c1cccc(C)c1] | 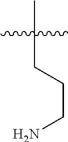 [SMILES: *CCCN] | 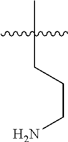 [SMILES: *CCCN] | H | H |
| 363 | 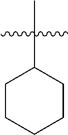 [SMILES: *C1CCCCC1] | 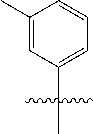 [SMILES: *c1cccc(C)c1] | H | 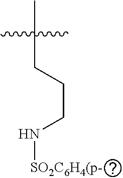 | H | H |
| 378 | 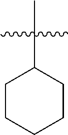 [SMILES: *C1CCCCC1] | 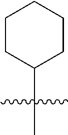 [SMILES: *C1CCCCC1] | H | 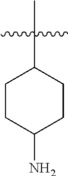 [SMILES: *C1CCC(N)CC1] | H | H |
| 379 | 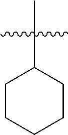 [SMILES: *C1CCCCC1] | 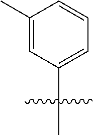 [SMILES: *c1cccc(C)c1] | H | 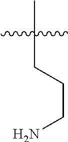 [SMILES: *CCCN] | CN | H |
| 382 | 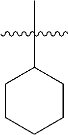 [SMILES: *C1CCCCC1] | 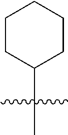 [SMILES: *C1CCCCC1] | H | 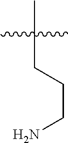 [SMILES: *CCCN] | CN | H |
| 383 | 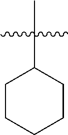 [SMILES: *C1CCCCC1] | 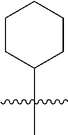 [SMILES: *C1CCCCC1] | H | 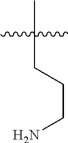 [SMILES: *CCCN] | H | H |
| 386 | 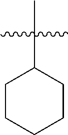 [SMILES: *C1CCCCC1] | 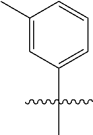 [SMILES: *c1cccc(C)c1] | H | 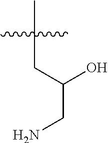 [SMILES: *CC(O)CN] | H | H |
| 388 | 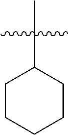 [SMILES: *C1CCCCC1] | 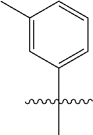 [SMILES: *c1cccc(C)c1] | H | 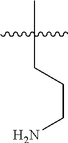 [SMILES: *CCCN] | CO₂H | H |
| 390 | 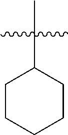 [SMILES: *C1CCCCC1] | 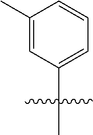 [SMILES: *c1cccc(C)c1] | H | 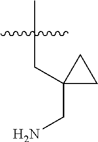 [SMILES: *CC1(CN)CC1] | H | H |
| 391 | 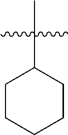 [SMILES: *C1CCCCC1] | 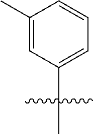 [SMILES: *c1cccc(C)c1] | H | Glucose | H | H |
| 392 | 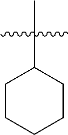 [SMILES: *C1CCCCC1] | 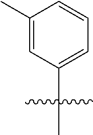 [SMILES: *c1cccc(C)c1] | H | 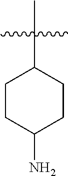 [SMILES: *C1CCC(N)CC1] | H | H |
| 373 | 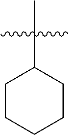 [SMILES: *C1CCCCC1] | 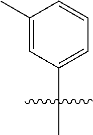 [SMILES: *c1cccc(C)c1] | H | 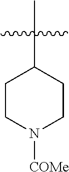 | H | H |
| 374 | 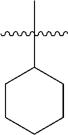 [SMILES: *C1CCCCC1] | 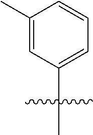 [SMILES: *c1cccc(C)c1] | H | 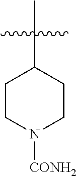 [SMILES: *C1CCN(C(N)=O)CC1] | H | H |
| 376 | 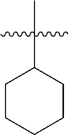 [SMILES: *C1CCCCC1] | 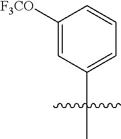 [SMILES: *c1cccc(OC(F)(F)F)c1] | H | 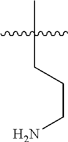 [SMILES: *CCCN] | CONH₂ | H |
| 377 | 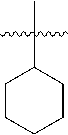 [SMILES: *C1CCCCC1] | 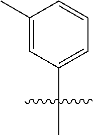 [SMILES: *c1cccc(C)c1] | H | 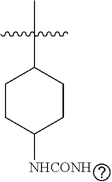 | H | H |
| 394 | 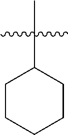 [SMILES: *C1CCCCC1] | 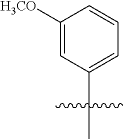 [SMILES: *c1cccc(OC)c1] | H | 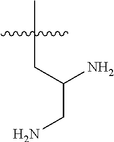 [SMILES: *CC(N)CN] | H | H |
| 400 | 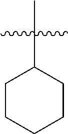 [SMILES: *C1CCCCC1] | 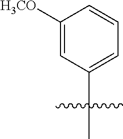 [SMILES: *c1cccc(OC)c1] | H | 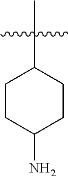 [SMILES: *C1CCC(N)CC1] | CN | H |
| 401 | 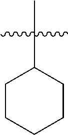 [SMILES: *C1CCCCC1] | 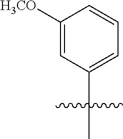 [SMILES: *c1cccc(OC)c1] | H | 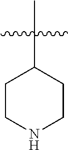 [SMILES: *C1CCNCC1] | CN | H |
| 402 | 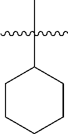 [SMILES: *C1CCCCC1] | 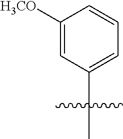 [SMILES: *c1cccc(OC)c1] | H | 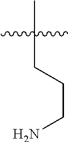 [SMILES: *CCCN] | CN | H |
| 405 | 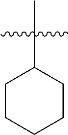 [SMILES: *C1CCCCC1] | 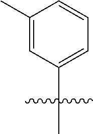 [SMILES: *c1cccc(C)c1] | H | 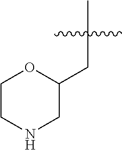 [SMILES: *CC1CNCCO1] | Br | H |
| 406 | 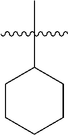 [SMILES: *C1CCCCC1] | 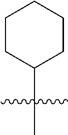 [SMILES: *C1CCCCC1] | H | 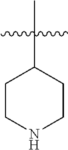 [SMILES: *C1CCNCC1] | CN | H |
| 408 | 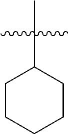 [SMILES: *C1CCCCC1] | 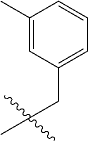 [SMILES: *Cc1cccc(C)c1] | H | 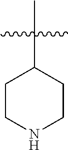 [SMILES: *C1CCNCC1] | H | H |
| | 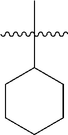 [SMILES: *C1CCCCC1] | 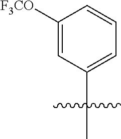 [SMILES: *c1cccc(OC(F)(F)F)c1] | H | 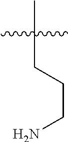 [SMILES: *CCCN] | H | CN |
| 413 | 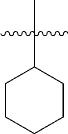 [SMILES: *C1CCCCC1] | 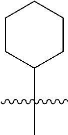 [SMILES: *C1CCCCC1] | H | 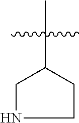 [SMILES: *C1CCNC1] | CN | H |
| 393 | 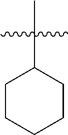 [SMILES: *C1CCCCC1] | 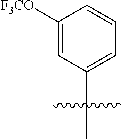 [SMILES: *c1cccc(OC(F)(F)F)c1] | H | 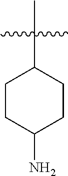 [SMILES: *C1CCC(N)CC1] | H | H |
| 415 | 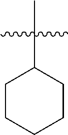 [SMILES: *C1CCCCC1] | 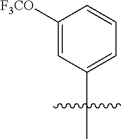 [SMILES: *c1cccc(OC(F)(F)F)c1] | H | 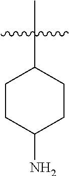 [SMILES: *C1CCC(N)CC1] | H | CN |
| 416 | 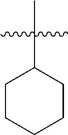 [SMILES: *C1CCCCC1] | 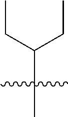 [SMILES: *C(CC)CC] | H | 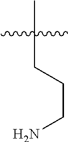 [SMILES: *CCCN] | H | H |
| 417 | C8H17 | 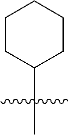 [SMILES: *C1CCCCC1] | H | 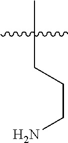 [SMILES: *CCCN] | H | H |
| 419 | 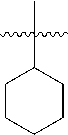 [SMILES: *C1CCCCC1] | 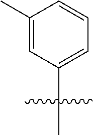 [SMILES: *c1cccc(C)c1] | H | 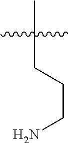 [SMILES: *CCCN] | H | H |
| 421 | Alkene | 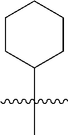 [SMILES: *C1CCCCC1] | H | 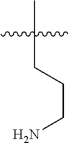 [SMILES: *CCCN] | H | H |
| 430 | 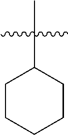 [SMILES: *C1CCCCC1] | 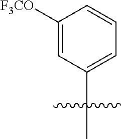 [SMILES: *c1cccc(OC(F)(F)F)c1] | 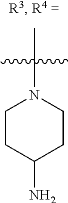 | | CN | H |
| 432 | 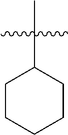 [SMILES: *C1CCCCC1] | 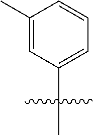 [SMILES: *c1cccc(C)c1] | H | 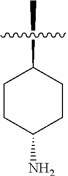 | NO₂ | H |
| 434 | 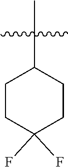 [SMILES: *C1CCC(F)(F)CC1] | 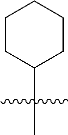 [SMILES: *C1CCCCC1] | H | 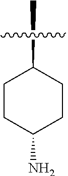 | CN | H |
| 414 | 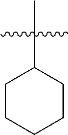 [SMILES: *C1CCCCC1] | 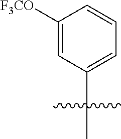 [SMILES: *c1cccc(OC(F)(F)F)c1] | H | 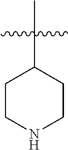 [SMILES: *C1CCNCC1] | H | CN |
| 422 | 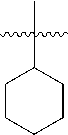 [SMILES: *C1CCCCC1] | 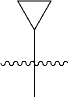 [SMILES: *C1CC1] | H | 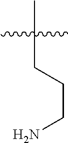 [SMILES: *CCCN] | H | H |
| 423 | 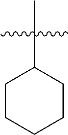 [SMILES: *C1CCCCC1] | 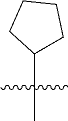 [SMILES: *C1CCCC1] | H | 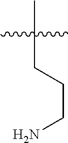 [SMILES: *CCCN] | H | H |
| 426 | C₈H₁₇ | 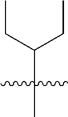 [SMILES: *C(CC)CC] | H | 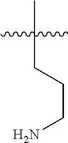 [SMILES: *CCCN] | H | H |
| 427 | C₈H₁₇ | 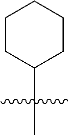 [SMILES: *C1CCCCC1] | H | 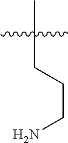 [SMILES: *CCCN] | H | H |
| 429 | 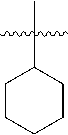 [SMILES: *C1CCCCC1] | 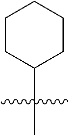 [SMILES: *C1CCCCC1] | H | 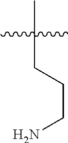 [SMILES: *CCCN] | H | Br |
| 431 | 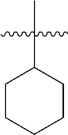 [SMILES: *C1CCCCC1] | 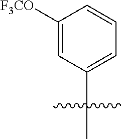 [SMILES: *c1cccc(OC(F)(F)F)c1] | H | 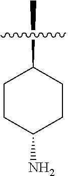 | CN | H |
| 433 | 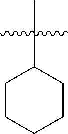 [SMILES: *C1CCCCC1] | 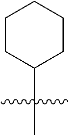 [SMILES: *C1CCCCC1] | H | 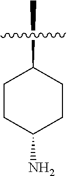 | F | H |
| 435 | 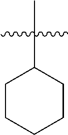 [SMILES: *C1CCCCC1] | 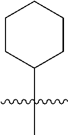 [SMILES: *C1CCCCC1] | H | 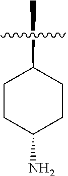 | Br | H |

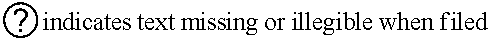

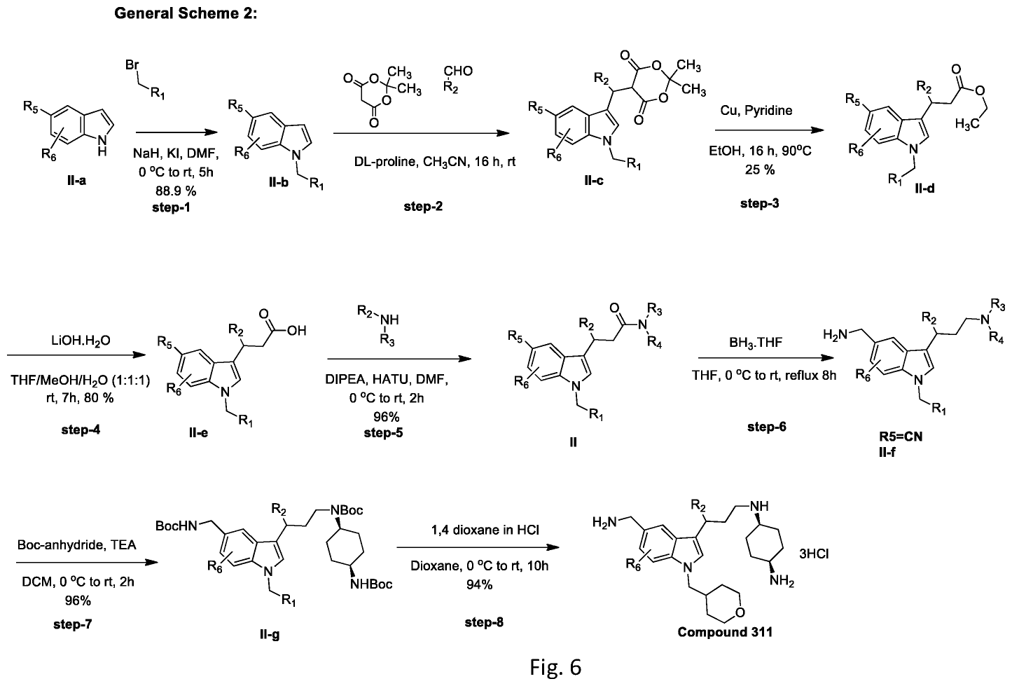

The general scheme 2 (FIG. 6) illustrates synthetic route of compound F-II and II. Alkylation of II-a with respective R₁CH₂X (X=leaving group) indole derivative II-b, which was coupled with aldehyde and cyclic ester, followed by decarboxylation gave ester derivative II-d. Ester hydrolysis of II-d followed by coupling with amines under coupling reagent provide compound of formula II or compound II with protecting group. Finally, deprotection under gave free base or its salt depending reaction condition. Depending on mature of R₅ various common functional group transformation was carried out. For example if, R₅=CN, then reduction of II under BH₃ gave II-f which was treated with (Boc)₂O to give II-g. Compound XX wad obtained by deprotection of Boc group under acidic condition. If R₃ and R₄ contain N and O protecting group, which can be deprotected under various condition reported in literature to obtain final compound of formula F-II or II listed in table 2.

### Example II: Synthesis of (1S,4S)-N1-(3-(5-(aminomethyl)-1-((tetrahydro-2H-pyran-4-yl) methyl)-1H-indol-3-yl)-3-(m-tolyl)propyl)cyclohexane-1,4-diamine

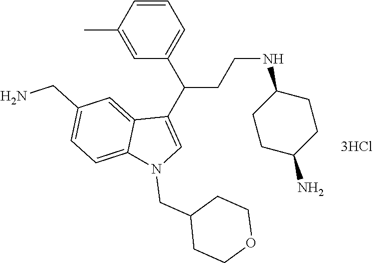 [SMILES: Cc1cccc(C(CCN[C@H]2CC[C@@H](N)CC2)c2cn(CC3CCOCC3)c3ccc(CN)cc23)c1]

### Synthesis of 1-((tetrahydro-2H-pyran-4-yl)methyl)-1H-indole-5-carbonitrile

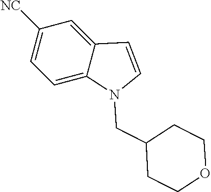 [SMILES: N#Cc1ccc2c(ccn2CC2CCOCC2)c1]

To a stirred solution of 1H-indole-5-carbonitrile (1.5 g, 10.56 mmol) in DMF (8 mL) were added KI (1.75 g, 10.56 mmol) followed by NaH (1.26 g, 31.68 mmol) in portion wise at 0° C. and reaction mixture was stirred at the same temperature for 5 min. After 5 min, 4-(bromomethyl) tetrahydro-2H-pyran (2.1 mL, 15.84 mmol) was added to reaction mixture at 0° C. then stirred at rt for 4 h. Progress of the reaction was monitored by TLC. Reaction mixture was quenched with crushed ice stirred for 15 min solid obtained in the reaction mixture was filtered off, dried under vacuum to get the pale cream solid (yield: 2.25 g, 88.9%).

¹H NMR (400 MHz, CDCl₃) δ 8.0 (s, 1H), 7.47-7.36 (m, 2H), 7.18 (d, J=3.14 Hz, 1H), 6.58 (d, J=3.0 Hz, 1H), 4.02 (d, J=7.29 Hz, 2H), 3.98 (d, J=3.38 Hz, 2H), 3.38-3.28 (m, 2H), 2.10-2.05 (m, 1H), 1.51-1.40 (m, 4H),

LC-MS m/z (M): calculated 240; found (M+H): 241

### Synthesis of 3-((2,2-dimethyl-4,6-dioxo-1,3-dioxan-5-yl) (m-tolyl)methyl)-1-((tetrahydro-2H-pyran-4-yl)methyl)-1H-indole-5-carbonitrile

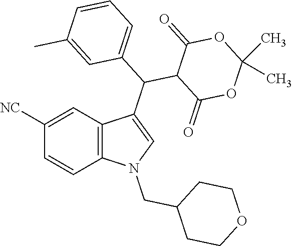 [SMILES: Cc1cccc(C(c2cn(CC3CCOCC3)c3ccc(C#N)cc23)C2C(=O)OC(C)(C)OC2=O)c1]

To a stirred solution of 1-((tetrahydro-2H-pyran-4-yl) methyl)-1H-indole-5-carbonitrile (2.2 g, 9.166 mmol) in dry Acetonitrile (20 mL), were added Meldrum's acid (2.63 g, 18.33 mmol), m-tolualdehyde (1.4 mL, 11.91 mmol) and DL-proline (105.3 mg, 0.916 mmol) then reaction mixture was stirred at rt for 16 h. Progress of the reaction was monitored by TLC. The reaction mixture was concentrated under vacuum and the crude product was carried forward to next step without purification (crude wt: 5.6 g)

LC-MS m/z (M): calculated 486.5; found (M+H): 487.3

### Synthesis of Ethyl 3-(5-cyano-1-((tetrahydro-2H-pyran-4-yl) methyl)-1H-indol-3-yl)-3-(m-tolyl)propanoate

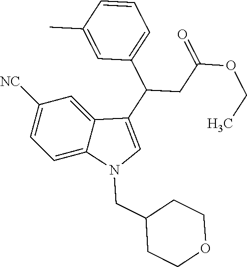 [SMILES: CCOC(=O)CC(c1cccc(C)c1)c1cn(CC2CCOCC2)c2ccc(C#N)cc12]

To a stirred solution of 3-((2,2-dimethyl-4,6-dioxo-1,3-dioxan-5-yl) (m-tolyl)methyl)-1-(tetrahydro-2H-pyran-4-yl)methyl)-1H-indole-5-carbonitrile (5.6 g, 11.5 mmol) in a 1:1 mixture of pyridine and Ethanol (60 mL) was added Cu powder (147 mg, 2.30 mmol) and stirred the reaction mixture at 90° C. for 16 h. The progress of the reaction was monitored by TLC. The reaction mixture was filtered and the filtrate was concentrated under reduced pressure. The crude compound was purified by column chromatography (silica gel 60-120 mesh, eluted with 10% EtOAc in Pet-ether) to afford desired product as yellow solid (yield: 950 mg, 25%).

¹H NMR (400 MHz, CDCl₃) δ 7.41 (d, J=7.92 Hz, 1H), 7.31 (d, J=8.25 Hz, 1H), 7.17-7.05 (m, 5H), 7.01-6.9 (m, 2H), 4.74 (t, J=7.91 Hz, 1H), 4.20-4.12 (m, 1H), 4.04-3.95 (m, 2H), 3.10-3.05 (m, 1H), 2.28 (s, 3H), 2.15-2.10 (m, 2H), 1.94-1.90 (m, 2H), 1.80-1.62 (m, 3H), 1.50-1.41 (m, 2H), 1.32-1.24 (m, 5H), 1.26 (t, J=3.5 Hz, 3H),

LC-MS m/z (M): calculated 430.54; found (M+H): 430.9

### Synthesis of 3-(5-cyano-1-((tetrahydro-2H-pyran-4-yl) methyl)-1H-indol-3-yl)-3-(m-tolyl)propanoic Acid

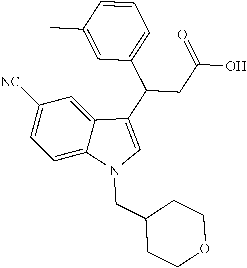 [SMILES: Cc1cccc(C(CC(=O)O)c2cn(CC3CCOCC3)c3ccc(C#N)cc23)c1]

To a stirred solution of ethyl 3-(5-cyano-1-((tetrahydro-2H-pyran-4-yl) methyl)-1H-indol-3-yl)-3-(m-tolyl)propanoate (400 mg, 0.930 mmol) in THF/MeOH/H₂O (1:1:1) (12 mL) was added LiOH.H₂O (390 mg, 9.30 mmol) at 0° C. and the reaction mixture was stirred at rt for 7 h. Progress of the reaction was monitored by TLC. The reaction mixture was acidified to pH 6 with citric acid, extracted with EtOAc, separated organic layer was dried over Na₂SO₄ and concentrated under reduced pressure. The crude compound was purified by column chromatography, eluted with 80 % EtOAc in hexane to afford pale cream solid (yield: 300 mg, 80%).

¹H NMR (400 MHz, CDCl₃) δ 7.71 (s, 1H), 7.40-7.28 (m, 2H), 7.17 (t, J=7.47 Hz, 1H), 7.09-7.04 (m, 4H), 4.70 (t, J=7.81 Hz, 1H), 4.01-3.92 (m, 4H), 3.35-3.28 (m, 2H), 3.12-3.0 (m, 2H), 2.30 (s, 3H), 2.09-2.0 (m, 1H), 1.5-1.25 (m, 5H),

LC-MS m/z (M): calculated 402.49; found (M−H): 401.1

### Synthesis of Tert-Butyl ((1S,4S)-4-(3-(5-cyano-1-((tetrahydro-2H-pyran-4-yl) methyl)-1H-indol-3-yl)-3-(m-tolyl)propanamido)cyclohexyl)carbamate

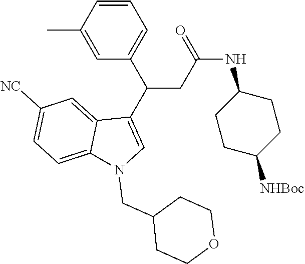 [SMILES: Cc1cccc(C(CC(=O)N[C@H]2CC[C@@H](NC(=O)OC(C)(C)C)CC2)c2cn(CC3CCOCC3)c3ccc(C#N)cc23)c1]

To a stirred solution of 3-(5-cyano-1-((tetrahydro-2H-pyran-4-yl) methyl)-1H-indol-3-yl)-3-(m-tolyl)propanoic acid (350 mg, 0.870 mmol) in DMF (3 mL), were added DIPEA (0.32 mL, 1.305 mmol), HATU (495 mg, 1.305 mmol) followed by tert-butyl ((1s,4s)-4-aminocyclohexyl)carbamate (242.5 mg, 1.131 mmol) at 0° C. and the reaction mixture was stirred at rt for 2 h. Progress of the reaction was monitored by TLC. Ice cold water added to reaction mixture at 0° C., extracted with EtOAc. The combined organic layer dried over Na₂SO₄ and concentrated under reduced pressure. The crude compound was purified by column chromatography, eluted with 70% EtOAc in Pet-ether to afford desired product as off white solid (yield: 500 mg, 96%).

¹H NMR (400 MHz, DMSO-d6) δ 7.71 (s, 1H), 7.39 (d, J=8.59 Hz, 1H), 7.31 (d, J=8.59 Hz, 1H), 7.18 (t, J=7.42 Hz, 1H), 7.10 (d, J=5.87 Hz, 2H), 7.04 (d, J=7.53 Hz, 2H), 4.66 (t, J=7.7 Hz, 1H), 4.28 (d, J=7.04 Hz, 1H), 4.0-3.95 (m, 4H), 3.80-3.71 (m, 2H), 3.45 (brs, 1H), 3.35-3.30 (m, 2H), 2.90-2.80 (m, 2H), 2.30 (s, 3H), 2.05-2.0 (m, 2H), 1.52-1.40 (m, 21H),

LC-MS m/z (M): calculated 598.7; found (M-Boc): 499.2

### Synthesis of Tert-Butyl ((1S,4S)-4-((3-(5-(aminomethyl)-1-((tetrahydro-2H-pyran-4-yl)methyl)-1H-indol-3-yl)-3-(m-tolyl)propyl)amino)cyclohexyl)carbamate

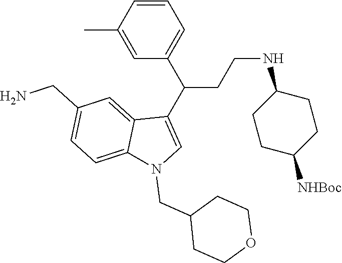 [SMILES: Cc1cccc(C(CCN[C@H]2CC[C@@H](NC(=O)OC(C)(C)C)CC2)c2cn(CC3CCOCC3)c3ccc(CN)cc23)c1]

To a stirred solution of tert-butyl ((1S,4S)-4-(3-(5-cyano-1-((tetrahydro-2H-pyran-4-yl) methyl)-1H-indol-3-yl)-3-(m-tolyl)propanamido)cyclohexyl)carbamate (300 mg, 0.501) in dry THE (6 mL), was added BH₃ in THF (1 M, 10 mL, 10.00 mmol)) at 0° C. and the reaction mixture was refluxed for 8 h. Progress of the reaction was monitored by TLC. After 8 h of reflux, 5 mL of MeOH was added then refluxed for 5 h. Solvent was removed from reaction mixture under reduced pressure and the crude compound was directly carry forward to the next step without further purification (crude wt: 450 mg).

### Synthesis of Tert-Butyl ((1S,4S)-4-((tert-butoxycarbonyl) amino)cyclohexyl)(3-(5-(((tert-butoxycarbonyl)amino)methyl)-1-((tetrahydro-2H-pyran-4-yl)methyl)-1H-indol-3-yl)-3-(m-tolyl)propyl)carbamate

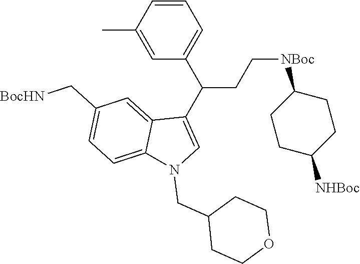

To a stirred solution of tert-butyl ((1S,4S)-4-((3-(5-(aminomethyl)-1-((tetrahydro-2H-pyran-4-yl)methyl)-1H-indol-3-yl)-3-(m-tolyl)propyl)amino)cyclohexyl)carbamate (450 mg, 0.765 mmol), were added TEA (0.55 mL, 3.825 mmol), followed by Boc anhydride (0.66 mL, 3.061 mmol), and the reaction mixture was stirred at rt for 12 h. Progress of the reaction was monitored by TLC. Excess solvent was removed from the reaction mixture and the crude compound was purified by column chromatography using 20% EtOAc in Hexane as an eluent to afford desired compound as brown liquid (yield: 120 mg, 30%).

¹H NMR (400 MHz, DMSO-d6) δ 7.35 (d, J=8.7 Hz, 1H), 7.30-7.25 (m, 2H), 7.24-7.20 (m, 3H), 7.0-6.0 (m, 2H), 4.10-4.05 (m, 2H), 4.04-3.99 (m, 3H), 3.80-3.65 (m, 4H), 3.21-3.05 (m, 3H), 3.0-2.91 (m, 1H), 2.21 (s, 3H), 2.02-1.95 (m, 1H), 1.69-1.60 (m, 2H), 1.51-1.42 (m, 5H), 1.42-1.30 (m, 22H), 1.30-1.20 (m, 8H),

LC-MS m/z (M): calculated 789; found (M-Boc): 689

### Synthesis of (1S,4S)-N1-(3-(5-(aminomethyl)-1-((tetrahydro-2H-pyran-4-yl)methyl)-1H-indol-3-yl)-3-(m-tolyl)propyl)cyclohexane-1,4-diamine

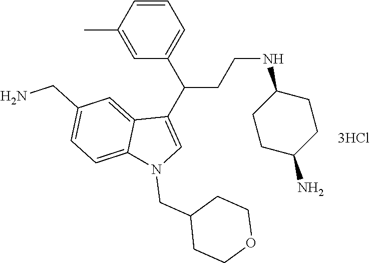 [SMILES: Cc1cccc(C(CCN[C@H]2CC[C@@H](N)CC2)c2cn(CC3CCOCC3)c3ccc(CN)cc23)c1]

To a stirred solution of tert-butyl ((s,4s)-4-((tert-butoxycarbonyl) amino)cyclohexyl)(3-(5-(((tert-butoxycarbonyl)amino)methyl)-1-((tetrahydro-2H-pyran-4-yl)methyl)-1H-indol-3-yl)-3-(m-tolyl)propyl)carbamate (120 mg, 0.152) in DCM (1.2 mL), was added 4 M HCl in 1,4-dioxane (1.2 mL) at 0° C. and reaction mixture was stirred at rt for 10 h. Progress of the reaction was monitored by TLC. Excess solvent was removed under reduced pressure and washed with diethyl ether to get off white solid (yield: 80 mg, 94%). MP: 130-134° C.

¹H NMR (400 MHz, DMSO-d6) δ 9.28 (brs, 1H), 9.17 (brs, 1H), 8.96 (brs, 2H), 8.30 (brs, 3H), 8.12 (brs, 1H), 7.66 (s, 1H), 7.51 (d, J=8.47 Hz, 1H), 7.46 (s, 1H), 7.21 (d, J=8.47 Hz, 1H), 7.18-7.14 (m, 3H), 6.97 (d, J=5.88 Hz, 1H), 4.23-4.19 (m, 1H), 4.10-4.01 (m, 4H), 3.79 (d, J=10.73 Hz, 2H), 3.21-3.10 (m, 4H), 2.95-2.84 (m, 2H), 2.72-2.65 (m, 1H), 2.40-2.38 (m, 1H), 2.24 (s, 3H), 2.10-1.98 (m, 3H), 1.90-1.60 (m, 6H), 1.85-1.20 (m, 4H)

LC-MS m/z (M): calculated 488.3; found (M+H): 489.3

Following the procedure described in scheme 2/Example II, compounds of Table 2 are prepared by using suitable starting materials and proper conditions.

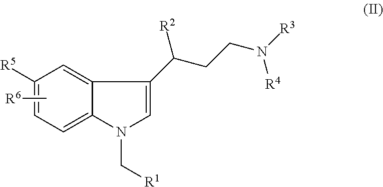

TABLE 2

| Cmpd | R¹ | R² | R³ | R⁴ | R⁵ | R⁶ |
|---|---|---|---|---|---|---|
| 311 | 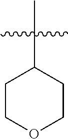 [SMILES: *C1CCOCC1] | 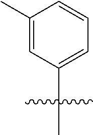 [SMILES: *c1cccc(C)c1] | H | 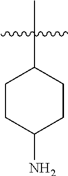 [SMILES: *C1CCC(N)CC1] | CH₂ NH₂ | H |
| 312 | 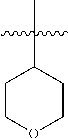 [SMILES: *C1CCOCC1] | 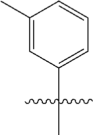 [SMILES: *c1cccc(C)c1] | H | 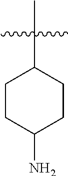 [SMILES: *C1CCC(N)CC1] | CN | H |
| 313 | 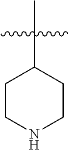 [SMILES: *C1CCNCC1] | 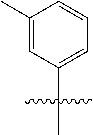 [SMILES: *c1cccc(C)c1] | H | 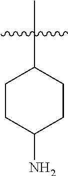 [SMILES: *C1CCC(N)CC1] | H | H |
| 314 | 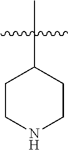 [SMILES: *C1CCNCC1] | 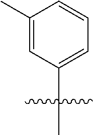 [SMILES: *c1cccc(C)c1] | H | 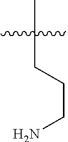 [SMILES: *CCCN] | H | H |
| 315 | 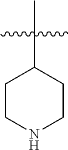 [SMILES: *C1CCNCC1] | 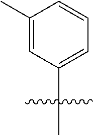 [SMILES: *c1cccc(C)c1] | H | 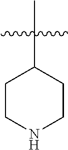 [SMILES: *C1CCNCC1] | H | H |
| 316 | 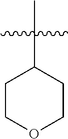 [SMILES: *C1CCOCC1] | 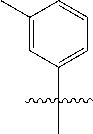 [SMILES: *c1cccc(C)c1] | H | 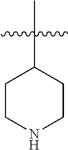 [SMILES: *C1CCNCC1] | H | H |
| 318 | 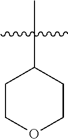 [SMILES: *C1CCOCC1] | 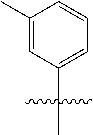 [SMILES: *c1cccc(C)c1] | H | 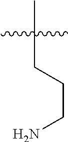 [SMILES: *CCCN] | CN | H |
| 425 | 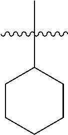 [SMILES: *C1CCCCC1] | 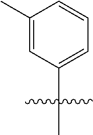 [SMILES: *c1cccc(C)c1] | H | 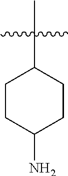 [SMILES: *C1CCC(N)CC1] | H | H |
| 428 | 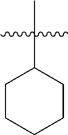 [SMILES: *C1CCCCC1] | 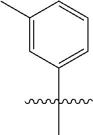 [SMILES: *c1cccc(C)c1] | H | 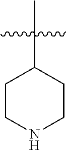 [SMILES: *C1CCNCC1] | NO₂ | H |
| 319 | 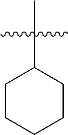 [SMILES: *C1CCCCC1] | 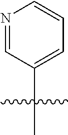 [SMILES: *c1cccnc1] | H | 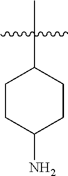 [SMILES: *C1CCC(N)CC1] | H | H |
| 320 | 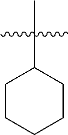 [SMILES: *C1CCCCC1] | 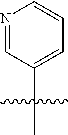 [SMILES: *c1cccnc1] | H | 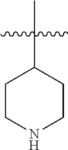 [SMILES: *C1CCNCC1] | H | H |
| 321 | 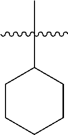 [SMILES: *C1CCCCC1] | 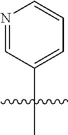 [SMILES: *c1cccnc1] | H | 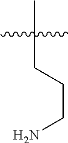 [SMILES: *CCCN] | H | H |
| 326 | 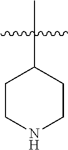 [SMILES: *C1CCNCC1] | 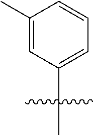 [SMILES: *c1cccc(C)c1] | H | 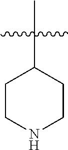 [SMILES: *C1CCNCC1] | CN | H |
| 327 | 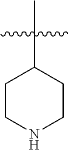 [SMILES: *C1CCNCC1] | 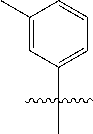 [SMILES: *c1cccc(C)c1] | H | 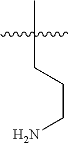 [SMILES: *CCCN] | CN | H |
| 346 | 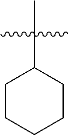 [SMILES: *C1CCCCC1] | 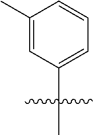 [SMILES: *c1cccc(C)c1] | H | 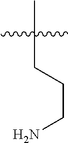 [SMILES: *CCCN] | H | H |
| 424 | 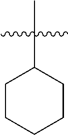 [SMILES: *C1CCCCC1] | 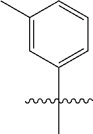 [SMILES: *c1cccc(C)c1] | H | 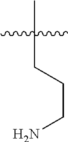 [SMILES: *CCCN] | NO₂ | H |
| 151 | 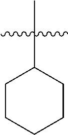 [SMILES: *C1CCCCC1] | 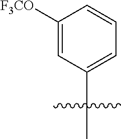 [SMILES: *c1cccc(OC(F)(F)F)c1] | H | 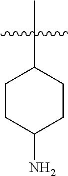 [SMILES: *C1CCC(N)CC1] | H | H |
| 152 | 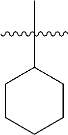 [SMILES: *C1CCCCC1] | 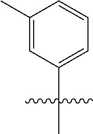 [SMILES: *c1cccc(C)c1] | H | 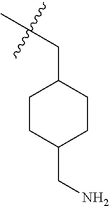 [SMILES: *CC1CCC(CN)CC1] | H | H |
| 122 | 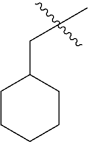 [SMILES: *CC1CCCCC1] | 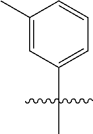 [SMILES: *c1cccc(C)c1] | H | 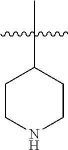 [SMILES: *C1CCNCC1] | H | H |
| 169 | 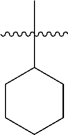 [SMILES: *C1CCCCC1] | 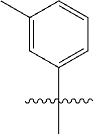 [SMILES: *c1cccc(C)c1] | H | 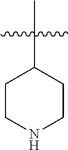 [SMILES: *C1CCNCC1] | Br | H |
| 165 | 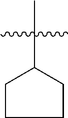 [SMILES: *C1CCCC1] | 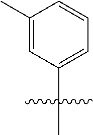 [SMILES: *c1cccc(C)c1] | H | 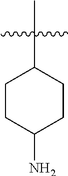 [SMILES: *C1CCC(N)CC1] | H | H |
| 150 | 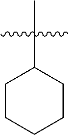 [SMILES: *C1CCCCC1] | 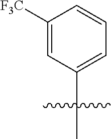 [SMILES: *c1cccc(C(F)(F)F)c1] | H | 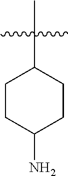 [SMILES: *C1CCC(N)CC1] | H | H |
| 171 | 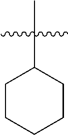 [SMILES: *C1CCCCC1] | 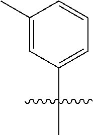 [SMILES: *c1cccc(C)c1] | H | 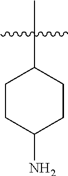 [SMILES: *C1CCC(N)CC1] | Br | H |
| 170 | 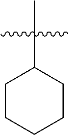 [SMILES: *C1CCCCC1] | 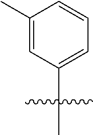 [SMILES: *c1cccc(C)c1] | H | 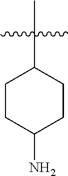 [SMILES: *C1CCC(N)CC1] | H | H |
| 160 | 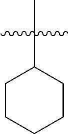 [SMILES: *C1CCCCC1] | 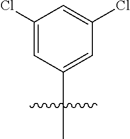 [SMILES: *c1cc(Cl)cc(Cl)c1] | | 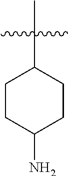 [SMILES: *C1CCC(N)CC1] | H | H |
| 161 | 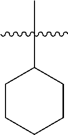 [SMILES: *C1CCCCC1] | 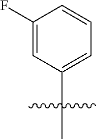 [SMILES: *c1cccc(F)c1] | H | 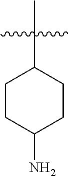 [SMILES: *C1CCC(N)CC1] | H | H |
| 178 | 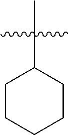 [SMILES: *C1CCCCC1] | 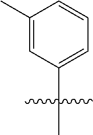 [SMILES: *c1cccc(C)c1] | H | 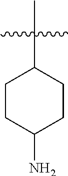 [SMILES: *C1CCC(N)CC1] | H | Br |
| 167 | Adamantyl | 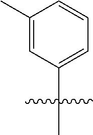 [SMILES: *c1cccc(C)c1] | H | 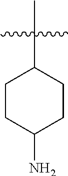 [SMILES: *C1CCC(N)CC1] | H | H |
| 166 | 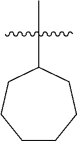 [SMILES: *C1CCCCCC1] | 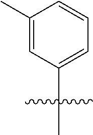 [SMILES: *c1cccc(C)c1] | H | 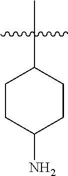 [SMILES: *C1CCC(N)CC1] | H | H |
| 157 | 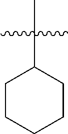 [SMILES: *C1CCCCC1] | 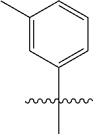 [SMILES: *c1cccc(C)c1] | H | 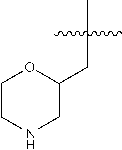 [SMILES: *CC1CNCCO1] | H | H |
| 186 | 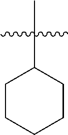 [SMILES: *C1CCCCC1] | 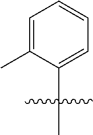 [SMILES: *c1ccccc1C] | H | 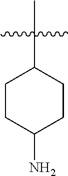 [SMILES: *C1CCC(N)CC1] | H | H |
| 153 | 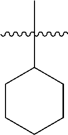 [SMILES: *C1CCCCC1] | 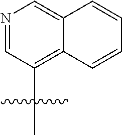 [SMILES: *c1cncc2ccccc12] | H | 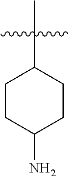 [SMILES: *C1CCC(N)CC1] | H | H |
| 199 | 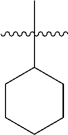 [SMILES: *C1CCCCC1] | 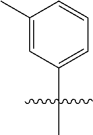 [SMILES: *c1cccc(C)c1] | H | 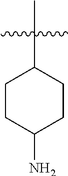 [SMILES: *C1CCC(N)CC1] | H | H |
| 154 | 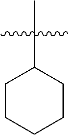 [SMILES: *C1CCCCC1] | 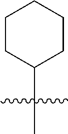 [SMILES: *C1CCCCC1] | H | 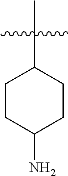 [SMILES: *C1CCC(N)CC1] | H | H |
| 174 | 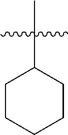 [SMILES: *C1CCCCC1] | H | H | 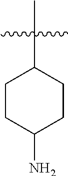 [SMILES: *C1CCC(N)CC1] | Br | H |
| 155 | 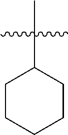 [SMILES: *C1CCCCC1] | 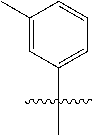 [SMILES: *c1cccc(C)c1] | H | 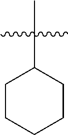 [SMILES: *C1CCCCC1] | H | H |
| 156 | 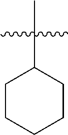 [SMILES: *C1CCCCC1] | 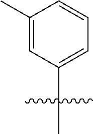 [SMILES: *c1cccc(C)c1] | 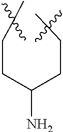 [SMILES: *CCC(N)CC*] | | H | H |
| 218 | 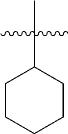 [SMILES: *C1CCCCC1] | 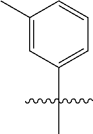 [SMILES: *c1cccc(C)c1] | H | 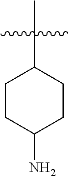 [SMILES: *C1CCC(N)CC1] | H | H |
| 175 | 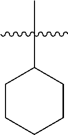 [SMILES: *C1CCCCC1] | H | H | 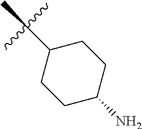 | 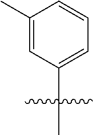 [SMILES: *c1cccc(C)c1] | H |
| 224 | 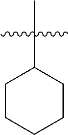 [SMILES: *C1CCCCC1] | 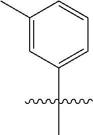 [SMILES: *c1cccc(C)c1] | H | 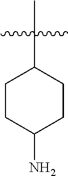 [SMILES: *C1CCC(N)CC1] | 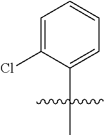 [SMILES: *c1ccccc1Cl] | H |
| 172 | 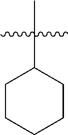 [SMILES: *C1CCCCC1] | 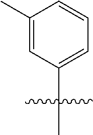 [SMILES: *c1cccc(C)c1] | H | 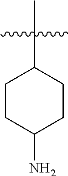 [SMILES: *C1CCC(N)CC1] | 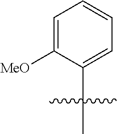 [SMILES: *c1ccccc1OC] | H |
| 177 | 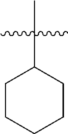 [SMILES: *C1CCCCC1] | H | H | 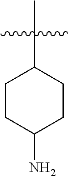 [SMILES: *C1CCC(N)CC1] | 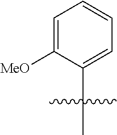 [SMILES: *c1ccccc1OC] | H |
| 168 | 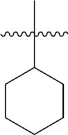 [SMILES: *C1CCCCC1] | 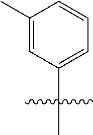 [SMILES: *c1cccc(C)c1] | H | 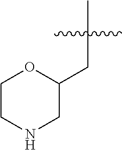 [SMILES: *CC1CNCCO1] | Br | H |
| 173 | 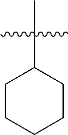 [SMILES: *C1CCCCC1] | 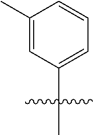 [SMILES: *c1cccc(C)c1] | Me | 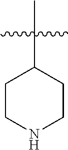 [SMILES: *C1CCNCC1] | Br | H |
| 164 | 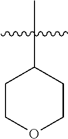 [SMILES: *C1CCOCC1] | 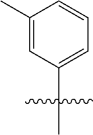 [SMILES: *c1cccc(C)c1] | H | 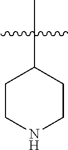 [SMILES: *C1CCNCC1] | H | H |
| 170 | 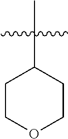 [SMILES: *C1CCOCC1] | 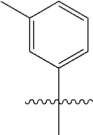 [SMILES: *c1cccc(C)c1] | H | 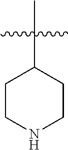 [SMILES: *C1CCNCC1] | Br | H |
| 176 | 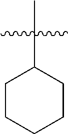 [SMILES: *C1CCCCC1] | H | H | 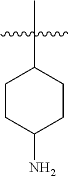 [SMILES: *C1CCC(N)CC1] | 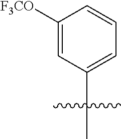 [SMILES: *c1cccc(OC(F)(F)F)c1] | H |
| 179 | 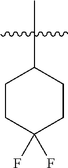 [SMILES: *C1CCC(F)(F)CC1] | H | H | 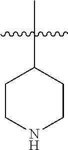 [SMILES: *C1CCNCC1] | Br | H |
| 375 | 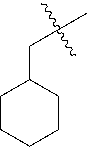 [SMILES: *CC1CCCCC1] | H | H | 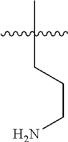 [SMILES: *CCCN] | 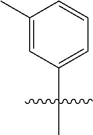 [SMILES: *c1cccc(C)c1] | H |

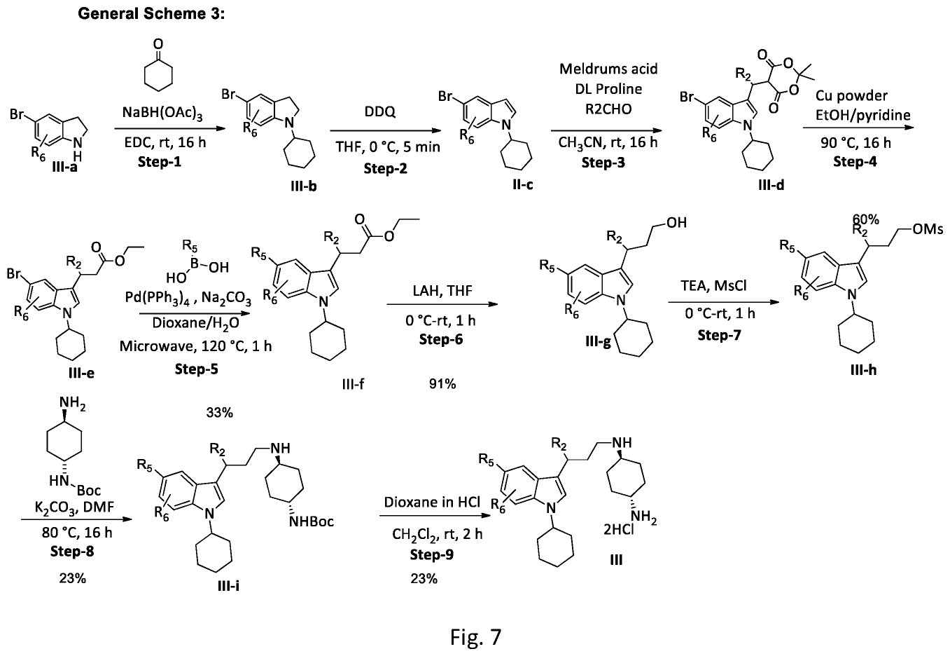

General Scheme 3 (FIG. 7) illustrates the synthetic routes for the synthesis of compounds of formula F-III and III. Reductive amination of III-a with ketone gave III-b which was oxidized with DDQ to provide indole derivative III-c. Coupling of Meldrum's acid and appropriate aldehyde R₂—CHO with III-c gave compound III-d, which under decarboxylation provide corresponding ester III-e. Suzuki coupling of III-e with appropriate boronic acid R₅—B(OH)₂ gave compound III-f followed by reduction of ester group gave corresponding alcohol III-g. Compound of formula III-h was obtained from III-g by nucleophilic reaction with MsCl, which was subjected to nucleophilic displacement with proper NHR₃R₄ to obtain III-j. Finally, deprotection of protecting group under acidic condition provide salt of compound II. If R₃ and R₄ contain N and O protecting group, which can be deprotected under various condition reported in literature to obtain final compound of formula F-III or III listed in table 3.

### Example 3: Synthesis of (1R,4R)-N1-(3-(1-cyclohexyl-5-(1-methyl-1H-pyrazol-5-yl)-1H-indol-3-yl)-3-(m-tolyl) propyl) cyclohexane-1,4-diamine Dihydrochloride

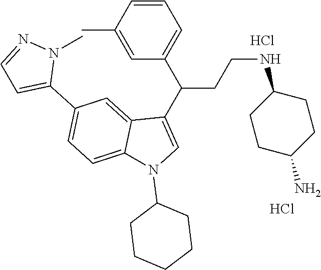 [SMILES: Cc1cccc(C(CCN[C@H]2CC[C@H](N)CC2)c2cn(C3CCCCC3)c3ccc(-c4ccnn4C)cc23)c1 ; Cl ; Cl]

### Step 1: 5-bromo-1-cyclohexylindoline

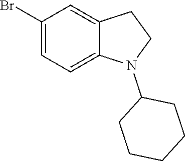 [SMILES: Brc1ccc2c(c1)CCN2C1CCCCC1]

To a stirred solution of 5-bromoindoline (10 g, 50.48 mmol, compound-1) in EDC (200 mL) was added cyclohexanone (15.8 ml-cyclohexyl-1H-indole-5-carbonitrile L, 151.46 mmol) at rt. After stirring the reaction mixture for 1 h was added NaBH(OAc)₃ (53.5 g, 252.41 mmol) and stirred the reaction mixture at rt for 16 h. The progress of the reaction was monitored by TLC. The reaction mixture was diluted with NaHCO₃ solution (100 mL), extracted with ethyl acetate (2×200 mL). The combined organic layer was dried over anhydrous sodium sulphate and concentrated under reduced pressure. The crude compound was purified column chromatography (silica gel 60-120 mesh, eluted with 2% EtOAc in pet ether) to afford 5-bromo-1-cyclohexylindoline (13.2 g, yield: 92%) as pale yellow liquid.

1H NMR (400 MHz, CDCl₃) δ 1.10-1.17 (m, 1H), 1.30-1.39 (m, 4H), 1.68 (d, J=12.7 Hz, 1H), 1.76-1.84 (m, 4H), 2.90 (t, J=8.4 Hz, 2H), 3.23-3.39 (m, 1H), 3.36 (t, J=8.4 Hz, 2H), 6.22-6.24 (m, 1H), 7.08-7.09 (m, 2H)

### Step 2: 5-bromo-1-cyclohexyl-1H-indole

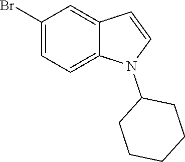 [SMILES: Brc1ccc2c(ccn2C2CCCCC2)c1]

To a stirred solution of 5-bromo-1-cyclohexylindoline (13 g, 46.55 mmol) in dry THF(130 mL) was added DDQ (11.6 g, 51.21 mmol) at 0° C. and stirred the reaction mixture at same temperature for 5 min. The progress of the reaction was monitored by TLC. The reaction mixture was diluted with water (20 mL), extracted with ethyl acetate (2×20 mL). The combined organic layer was dried over anhydrous sodium sulphate and concentrated under reduced pressure. The crude compound was purified column chromatography (silica gel 60-120 mesh, eluted with 2% EtOAc in pet-ether) to afford 5-bromo-1-cyclohexyl-1H-indole (10 g, yield: 77%) as light greenish liquid.

### Step 3: 5-((5-bromo-1-cyclohexyl-1H-indol-3-yl) (m-tolyl)methyl)-2,2-dimethyl-1,3-dioxane-4,6-dione

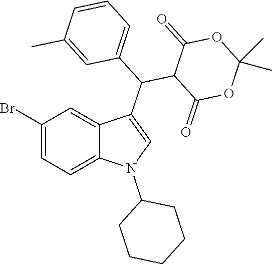 [SMILES: Cc1cccc(C(c2cn(C3CCCCC3)c3ccc(Br)cc23)C2C(=O)OC(C)(C)OC2=O)c1]

To a stirred solution of 5-bromo-1-cyclohexyl-1H-indole (5 g, 17.985 mmol) in CH₃CN (50 mL) was added m-Toulaldehyde (3.1 mL, 26.97 mmol), DL-proline (207 mg, 1.798 mmol) followed by Meldrum's acid (5.1 g, 35.971 mmol) and stirred the reaction mixture at rt for 16 h. The progress of the reaction was monitored by TLC. The reaction mixture was concentrated under reduced pressure to afford 5-((5-bromo-1-cyclohexyl-1H-indol-3-yl) (m-tolyl)methyl)-2,2-dimethyl-1,3-dioxane-4,6-dione (13 g, crude) as brown semi-solid. The crude compound was used in the next step.

LC-MS m/z (M−H): 429.4

### Step 4: ethyl 3-(5-bromo-1-cyclohexyl-1H-indol-3-yl)-3-(m-tolyl) Propanoate

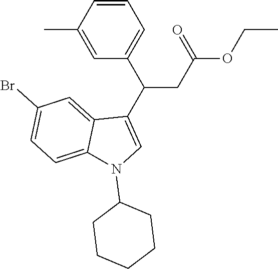 [SMILES: CCOC(=O)CC(c1cccc(C)c1)c1cn(C2CCCCC2)c2ccc(Br)cc12]

To a stirred solution of 5-((5-bromo-1-cyclohexyl-1H-indol-3-yl) (m-tolyl)methyl)-2,2-dimethyl-1,3-dioxane-4,6-dione (13 g, 24.787 mmol) in EtOH/pyridine (195 mL, 1:1 v/v) was added Cu powder (143 mg, 2.478 mmol) and stirred the reaction mixture at 90° C. for 16 h. The progress of the reaction was monitored by TLC. The reaction mixture was cooled to rt, filtered through, the filtrate was concentrated under reduced pressure. The crude compound was purified by combi-flash column chromatography (eluted with 10% EtOAc in pet ether) to afford ethyl 3-(5-bromo-1-cyclohexyl-1H-indol-3-yl)-3-(m-tolyl) propanoate (7 g, yield: 60%) as pale yellow semi-solid. 1H NMR (400 MHz, CDCl₃) δ 1.10 (t, J=2.1 Hz, 3H), 1.22-1.33 (m, 1H), 1.42-1.53 (m, 2H), 1.61-1.71 (m, 2H), 1.78 (d, J=13.1 Hz, 1H), 1.92 (d, J=13.3 Hz, 2H), 2.08 (s, 2H), 2.30 (s, 3H), 2.93-2.99 (m, 1H), 3.03-3.09 (m, 1H), 4.00-4.09 (m, 2H), 4.10-4.15 (m, 1H), 4.67 (t, J=7.9 Hz, 1H), 6.99 (d, J=7.3 Hz, 1H), 7.06-7.08 (m, 3H), 7.13-7.20 (m, 3H), 7.53 (d, J=1.5 Hz, 1H)

LC-MS m/z (M+H): 468.4

### Step 5: Ethyl 3-(5-bromo-1-cyclohexyl-1H-indol-3-yl)-3-(m-tolyl) Propanoate

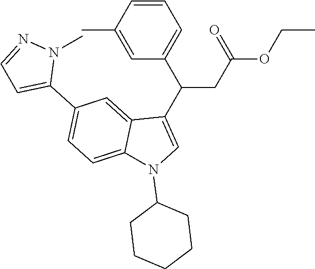 [SMILES: CCOC(=O)CC(c1cccc(C)c1)c1cn(C2CCCCC2)c2ccc(-c3ccnn3C)cc12]

To a stirred solution of ethyl 3-(5-bromo-1-cyclohexyl-1H-indol-3-yl)-3-(m-tolyl) propanoate (500 mg, 1.068 mmol) in Dioxane/H₂O (10 mL, 4:1 v/v) was added (1-methyl-1H-pyrazol-5-yl)boronic acid (161 mg, 1.282 mmol, Na₂CO₃ (339 mg, 3.205 mmol) at rt. After degassed for 10 min was added Pd(PPh₃)₄ (123 mg, 0.106 mmol) again degassed for 5 min and stirred the reaction mixture in microwave at 120° C. for 1 h. The progress of the reaction was monitored by TLC. The reaction mixture was filtered through a pad of celite, the filtrate was dried over anhydrous sodium sulphate and concentrated under reduced pressure. The crude compound was purified by combi-flash column chromatography (eluted with 13% EtOAc in pet ether) to afford ethyl 3-(5-bromo-1-cyclohexyl-1H-indol-3-yl)-3-(m-tolyl) propanoate (300 mg, yield: 33%) as pale yellow semi-solid.

LC-MS m/z (M+H): 470.3

### Step 6: 3-(1-cyclohexyl-5-(1-methyl-1H-pyrazol-5-yl)-1H-indol-3-yl)-3-(m-tolyl) propan-1-ol

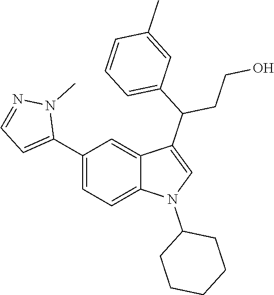 [SMILES: Cc1cccc(C(CCO)c2cn(C3CCCCC3)c3ccc(-c4ccnn4C)cc23)c1]

To a stirred solution of ethyl 3-(5-bromo-1-cyclohexyl-1H-indol-3-yl)-3-(m-tolyl) propanoate (300 mg, 0.639 mmol) in THF (6 mL) was added LAH (48 mg, 1.279 mmol) at 0° C. and stirred the reaction mixture at rt for 1 h. The progress of the reaction was monitored by TLC. The reaction mixture was slowly poured into Na₂SO₄ paste, filtered and the filtrate was dried over anhydrous sodium sulphate and concentrated under reduced pressure to afford 3-(1-cyclohexyl-5-(1-methyl-1H-pyrazol-5-yl)-1H-indol-3-yl)-3-(m-tolyl) propan-1-ol (250 mg, yield: 91%) as pale yellow semi-solid.

LC-MS m/z (M+H): 428.3

### Step 7: 3-(1-cyclohexyl-5-(1-methyl-1H-pyrazol-5-yl)-1H-indol-3-yl)-3-(m-tolyl) propyl Methanesulfonate

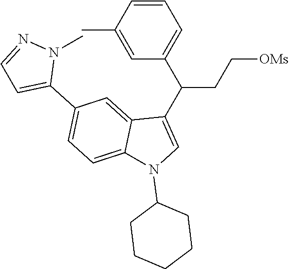 [SMILES: Cc1cccc(C(CCOS(C)(=O)=O)c2cn(C3CCCCC3)c3ccc(-c4ccnn4C)cc23)c1]

To a stirred solution of 3-(1-cyclohexyl-5-(1-methyl-H-pyrazol-5-yl)-1H-indol-3-yl)-3-(m-tolyl) propan-1-ol (250 mg, 0.585 mmol) in CH₂Cl₂ (5 mL) was added TEA (0.2 mL, 1.463 mmol) followed by MsCl (0.07 mL, 0.877 mmol) at 0° C. and stirred the reaction mixture at rt for 1 h. The progress of the reaction was monitored by TLC. The reaction mixture was diluted with water (10 mL), extracted with DCM (2×10 mL). The combined organic layer was washed with NaHCO₃ solution, dried over anhydrous sodium sulphate and concentrated under reduced pressure to afford 3-(1-cyclohexyl-5-(1-methyl-1H-pyrazol-5-yl)-1H-indol-3-yl)-3-(m-tolyl) propyl methanesulfonate (340 mg, crude) as yellow semi-solid. The crude compound was used in the next step.

### Step 8: Tert-Butyl ((1R,4R)-4-((3-(1-cyclohexyl-5-(1-methyl-1H-pyrazol-5-yl)-1H-indol-3-yl)-3-(m-tolyl) propyl)amino)cyclohexyl)carbamate

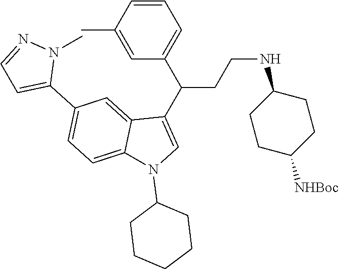 [SMILES: Cc1cccc(C(CCN[C@H]2CC[C@H](NC(=O)OC(C)(C)C)CC2)c2cn(C3CCCCC3)c3ccc(-c4ccnn4C)cc23)c1]

To a stirred solution of 3-(1-cyclohexyl-5-(1-methyl-H-pyrazol-5-yl)-1H-indol-3-yl)-3-(m-tolyl) propyl methane sulfonate (340 mg, 0.672 mmol) in DMF (5 mL) was added tert-butyl ((1R,4R)-4-aminocyclohexyl)carbamate (216 mg, 1.008 mmol) followed by K₂CO₃ (278 mg, 2.017 mmol) and stirred the reaction mixture at 80° C. for 16 h. The progress of the reaction was monitored by TLC. The reaction mixture was diluted with water (10 mL), filtered, the residue was dissolved in ethyl acetate (20 mL), dried over anhydrous sodium sulphate and concentrated under reduced pressure. The crude compound was purified by preparative TLC (5% MeOH/CH₂Cl₂) to afford tert-butyl ((1R,4R)-4-((3-(1-cyclohexyl-5-(1-methyl-1H-pyrazol-5-yl)-1H-indol-3-yl)-3-(m-tolyl) propyl)amino) cyclohexyl) carbamate (100 mg, yield: 23%) as yellow liquid.

LC-MS m/z (M+H): 624.3

### Step 9: (1R,4R)-N1-(3-(1-cyclohexyl-5-(1-methyl-1H-pyrazol-5-yl)-1H-indol-3-yl)-3-(m-tolyl) propyl) cyclohexane-1,4-diamine Dihydrochloride

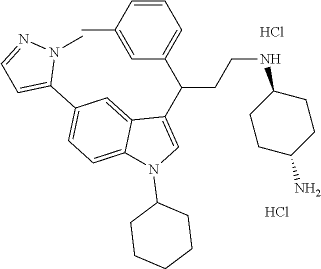 [SMILES: Cc1cccc(C(CCN[C@H]2CC[C@H](N)CC2)c2cn(C3CCCCC3)c3ccc(-c4ccnn4C)cc23)c1 ; Cl ; Cl]

To a stirred solution of tert-butyl ((1r,4r)-4-((3-(1-cyclohexyl-5-(1-methyl-H-pyrazol-5-yl)-1H-indol-3-yl)-3-(m-tolyl) propyl)amino)cyclohexyl)carbamate (70 mg, 0.113 mmol) in CH₂C12 (2 mL) was added HCl in Dioxane (2 mL) and stirred the reaction mixture at rt for 2 h. The progress of the reaction was monitored by TLC. The reaction mixture was concentrated under reduced pressure. The crude compound was washed with pentane (5 mL) to afford (1R,4R)-N1-(3-(1-cyclohexyl-5-(1-methyl-1H-pyrazol-5-yl)-1H-indol-3-yl)-3-(m-tolyl) propyl) cyclohexane-1,4-diamine dihydrochloride (16 mg, yield: 23%) as off white solid.

1HNMR (400 MHz, DMSO-d₆) δ 1.22-1.44 (m, 5H), 1.46-1.56 (m, 2H), 1.70-1.85 (m, 5H), 1.95-2.06 (m, 6H), 2.24 (s, 3H), 2.31 (s, 1H), 2.79 (s, 1H), 2.92 (s, 3H), 3.79 (s, 4H), 4.31-4.37 (m, 2H), 6.96 (s, 1H), 7.13-7.18 (m, 4H), 7.41 (d, J=1.5 Hz, 1H), 7.49 (s, 1H), 7.57 (d, J=8.5 Hz, 1H), 7.61 (s, 1H), 7.99 (s, 3H), 9.03 (s, 1H), 9.14 (s, 1H)

LC-MS m/z (M+H): 524.3

Following the procedure described in scheme 3/Example III, compounds of Table 3 are prepared by using suitable starting materials and proper conditions.

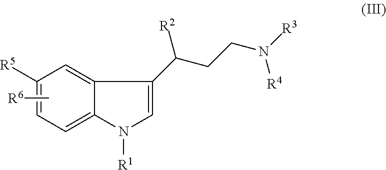

TABLE 3

| Cmpd | R¹ | R² | R³ | R⁴ | R⁵ | R⁶ |
|---|---|---|---|---|---|---|
| 362 | 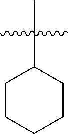 [SMILES: *C1CCCCC1] | H | H | 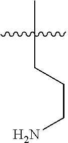 [SMILES: *CCCN] | 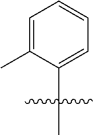 [SMILES: *c1ccccc1C] | H |
| 370 | 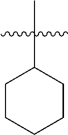 [SMILES: *C1CCCCC1] | H | H | 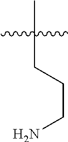 [SMILES: *CCCN] | 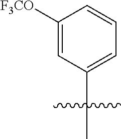 [SMILES: *c1cccc(OC(F)(F)F)c1] | H |
| 384 | 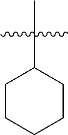 [SMILES: *C1CCCCC1] | H | H | 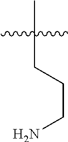 [SMILES: *CCCN] | 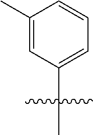 [SMILES: *c1cccc(C)c1] | H |
| 385 | 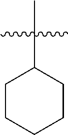 [SMILES: *C1CCCCC1] | H | H | 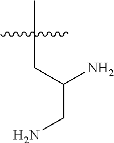 [SMILES: *CC(N)CN] | 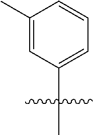 [SMILES: *c1cccc(C)c1] | H |
| 412 | 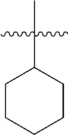 [SMILES: *C1CCCCC1] | 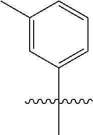 [SMILES: *c1cccc(C)c1] | H | 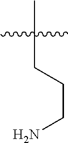 [SMILES: *CCCN] | 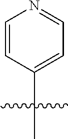 [SMILES: *c1ccncc1] | H |
| 399 | 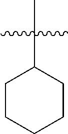 [SMILES: *C1CCCCC1] | H | H | 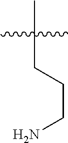 [SMILES: *CCCN] | 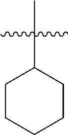 [SMILES: *C1CCCCC1] | H |
| 409 | 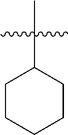 [SMILES: *C1CCCCC1] | 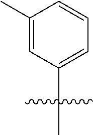 [SMILES: *c1cccc(C)c1] | H | 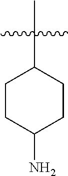 [SMILES: *C1CCC(N)CC1] | 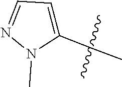 [SMILES: *c1ccnn1C] | H |
| 398 | 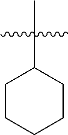 [SMILES: *C1CCCCC1] | H | H | 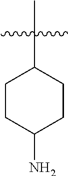 [SMILES: *C1CCC(N)CC1] | 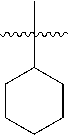 [SMILES: *C1CCCCC1] | H |

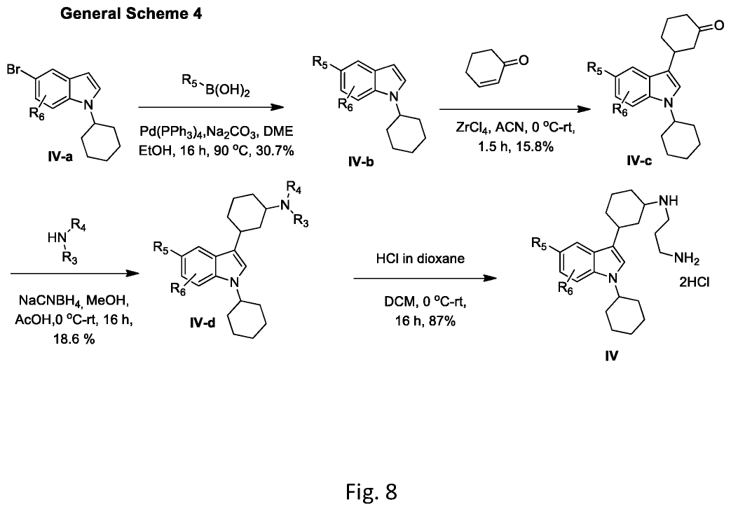

General scheme 4 (FIG. 8) shows for the synthesis of compound of formula IV. Suzuki coupling of IV-a with various boronic acid or ester like R₅—B(OH)₂ gave compounds of formula IV-b, which under Michael reaction under Lewis acid gave corresponding ketone IV-c. Reductive amination of IV-c gave corresponding amine IV-d. If R₃, R₄ contains protecting group then deprotection was carried out under acidic condition to provide salt of compound IV.

### Example 4: Synthesis of (3-((2-(1-cyclohexyl-5-(3-(trifluoromethoxy) phenyl-1H-indol-3-yl)ethyl)amino)propyl)carbamate Dihydrochloride

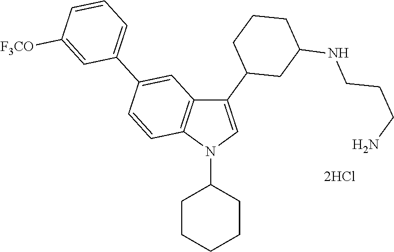 [SMILES: NCCCNC1CCCC(c2cn(C3CCCCC3)c3ccc(-c4cccc(OC(F)(F)F)c4)cc23)C1]

### Synthesis of 1-cyclohexyl-5-(3-(trifluoromethoxy) phenyl)-1H-indole

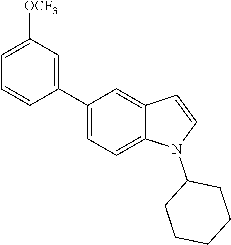 [SMILES: FC(F)(F)Oc1cccc(-c2ccc3c(ccn3C3CCCCC3)c2)c1]

To a stirred solution of 5-bromo-1-cyclohexyl-1H-indole (3 g, 10.791 mmol) in DME (39 mL), was added Pd(PPh₃)₄ (623 mg, 0.539 mmol) under nitrogen atmosphere and the reaction mixture was stirred at rt for 15 mins. After 15 mins, (3-(trifluoromethoxy) phenyl)boronic acid (2.22 g, 10.791 mmol) in EtOH (10 mL) was added to the reaction mixture and was stirred at rt again for 15 min. Finally, aq Na₂CO₃ (2 M) solution (39 mL) was added and the reaction mixture was stirred at 90° C. for 16 h. Progress of the reaction was monitored by TLC. Reaction mixture was cooled to rt, filtered through celite bed then filtrate was extracted with EtOAc (3×50 mL). The organic layer was dried over anhydrous sodium sulphate, concentrated under reduced pressure. The crude compound was purified by column chromatography using 2% EtOAc in Pet-ether as an eluent to afford desired product as colourless liquid (yield: 1.19 g, 30.7%).

¹H NMR (400 MHz, CDCl₃) δ 7.82 (s, 1H), 7.63-7.55 (m, 1H), 7.50-7.38 (m, 3H), 7.29-7.26 (m, 1H), 7.25-7.18 (m, 1H), 7.16-7.08 (m, 1H), 4.28-4.20 (m, 1H), 2.20-2.10 (m, 2H), 2.00-1.90 (m, 2H), 1.85-1.68 (m, 3H), 1.52-1.46 (m, 2H), 1.38-1.22 (m, 1H)

LC-MS m/z (M): calculated 359.3; found (M+H): 360.17

### Synthesis of 3-(1-cyclohexyl-5-(3-(trifluoromethoxy) phenyl)-1H-indol-3-yl)cyclohexanone

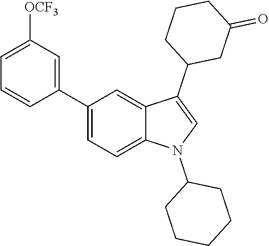 [SMILES: O=C1CCCC(c2cn(C3CCCCC3)c3ccc(-c4cccc(OC(F)(F)F)c4)cc23)C1]

To a stirred solution of 1-cyclohexyl-5-(3-(trifluoromethoxy) phenyl)-1H-indole (1.19 g, 3.311 mmol) in dry ACN (12 mL), were added cyclohex-2-enone (0.32 mL, 3.311 mmol) followed by ZrCl₄ at 0° C. and the reaction mixture was stirred at rt for 1.5 h. Reaction mixture was turned into blue colour and progress of the reaction was monitored by TLC. The reaction mixture was diluted with water, extracted with EtOAc, dried over sodium sulphate and concentrated under reduced pressure. The crude compound was purified by column chromatography using 6% EtOAc in Pet-ether as an eluent to afford desired product as brown colour liquid (yield: 238 mg, 15.8%).

¹H NMR (400 MHz, CDCl₃) δ 7.75 (s, 1H), 7.58-7.55 (m, 1H), 7.48-7.42 (m, 3H), 7.20-7.15 (m, 1H), 7.04 (s, 1H), 7.02-6.98 (m, 1H), 4.24-4.18 (m, 1H), 3.52-3.48 (m, 1H), 2.82-2.78 (m, 1H), 2.68-2.60 (m, 1H), 2.49-2.40 (m, 2H), 2.39-2.32 (m, 1H), 2.30-2.22 (m, 1H), 2.15-2.10 (m, 2H), 2.05-1.90 (m, 4H), 1.88-1.78 (m, 2H), 1.75-1.68 (m, 2H), 1.55-1.45 (m, 2H), 1.35-1.20 (m, 5H), 0.90-0.80 (m, 2H)

LC-MS m/z (M): calculated 455.51; found (M+H): 456.2

### Synthesis of Tert-Butyl (3-((3-(1-cyclohexyl-5-(3-(trifluoromethoxy) phenyl)-1H-indol-3-yl)cyclohexyl)amino)propyl)carbamate

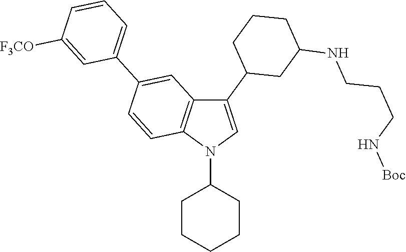 [SMILES: CC(C)(C)OC(=O)NCCCNC1CCCC(c2cn(C3CCCCC3)c3ccc(-c4cccc(OC(F)(F)F)c4)cc23)C1]

To a stirred solution of 3-(1-cyclohexyl-5-(3-(trifluoromethoxy)phenyl)-1H-indol-3-yl) cyclohexanone (120 mg, 0.263 mmol) in MeOH (3 mL), were added tert-butyl (3-aminopropyl) carbamate (59.6 mg, 0.342 mmol), AcOH (36.2 mg, 0.604 mmol) and reaction mixture was stirred at rt, after 1 h of stirring, was added NaCNBH₄ (33 mg, 0.526) at 0° C. then stirred the reaction mixture at rt for 16 h. Progress of the reaction was monitored by TLC. The reaction mixture was diluted with aq NaHCO₃ solution (10 mL) and compound was extracted with 10% MeOH in DCM (3×10 mL). The organic layer was dried over anhydrous sodium sulphate, concentrated under reduced pressure. The crude compound was purified by preparative HPLC method to afford desired product as colourless gummy mass (yield: 30 mg, 18.6%).

¹H NMR (400 MHz, CDCl₃) δ 7.81 (s, 1H), 7.72-7.68 (m, 1H), 7.60-7.52 (m, 3H), 7.40 (d, J=8.71 Hz, 1H), 7.30-7.22 (m, 2H), 6.82-6.78 (m, 1H), 4.32-4.28 (m, 1H), 3.02-2.88 (m, 3H), 2.60-2.55 (m, 2H), 2.20-2.18 (m, 1H), 2.0-1.90 (m, 4H), 1.88-1.78 (m, 3H), 1.75-1.68 (m, 3H), 1.15-1.45 (m, 5H), 1.38-1.36 (m, 1H), 1.32 (s, 9H), 1.25-1.20 (m, 6H)

LC-MS m/z (M): calculated 613.7; found (M+H): 614.23

### Synthesis of (3-((2-(1-cyclohexyl-5-(3-(trifluoromethoxy) phenyl-1H-indol-3-yl)ethyl)amino)propyl)carbamate Dihydrochloride

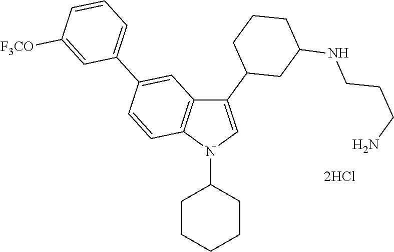 [SMILES: NCCCNC1CCCC(c2cn(C3CCCCC3)c3ccc(-c4cccc(OC(F)(F)F)c4)cc23)C1]

To a stirred solution of tert-butyl (3-((3-(1-cyclohexyl-5-(3-(trifluoromethoxy) phenyl)-1H-indol-3-yl)cyclohexyl)amino)propyl)carbamate (30 mg, 0.048 mmol) in CH₂C12 (1 mL), was added HCl in dioxane (4M, 1 mL) at 0° C. and the reaction mixture was stirred at room temperature for 2 h. The reaction mixture was concentrated under reduced pressure and the crude compound was washed with n-pentane to afford desired compound as off white solid (yield: 25 mg, 87%).

MP: 202-206° C.

¹H NMR (400 MHz, DMSO-d6) δ 9.05-9.02 (m, 2H), 8.80-8.74 (m, 2H), 7.89-7.88 (m, 1H), 7.74 (d, J=7.88 Hz, 1H), 7.61-7.45 (m, 3H), 7.44 (d, J=8.36 Hz, 1H), 7.30-7.28 (m, 2H), 4.33 (t, J=11.56 Hz, 1H), 3.59-3.52 (m, 2H), 2.42-2.38 (m, 1H), 2.25-2.10 (m, 3H), 2.0-1.90 (m, 4H), 1.89-1.80 (m, 5H), 1.75-1.62 (m, 3H), 1.60-1.40 (m, 6H), 1.32-1.30 (m, 1H),

LC-MS m/z (M): calculated 513.6; found (M+H): 514.33

Following the procedure described in scheme 4/Example IV, compounds of Table 4 are prepared by using suitable starting materials and proper conditions.

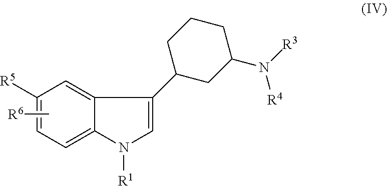

TABLE 4

| Cmpd | R¹ | R³ | R⁴ | R⁵ | R⁶ |
|---|---|---|---|---|---|
| 380 | 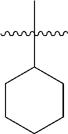 [SMILES: *C1CCCCC1] | H | 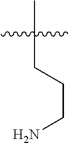 [SMILES: *CCCN] | Br | H |
| 381 | 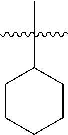 [SMILES: *C1CCCCC1] | H | 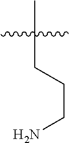 [SMILES: *CCCN] | 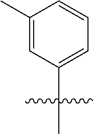 [SMILES: *c1cccc(C)c1] | H |
| 389 | 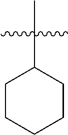 [SMILES: *C1CCCCC1] | H | 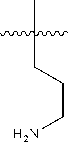 [SMILES: *CCCN] | CN | H |
| 395 | 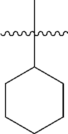 [SMILES: *C1CCCCC1] | H | 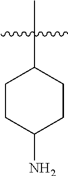 [SMILES: *C1CCC(N)CC1] | 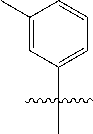 [SMILES: *c1cccc(C)c1] | H |
| 396 | 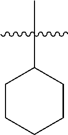 [SMILES: *C1CCCCC1] | H | 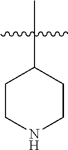 [SMILES: *C1CCNCC1] | 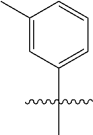 [SMILES: *c1cccc(C)c1] | H |
| 403 | 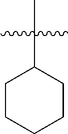 [SMILES: *C1CCCCC1] | H | 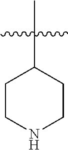 [SMILES: *C1CCNCC1] | 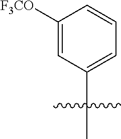 [SMILES: *c1cccc(OC(F)(F)F)c1] | H |
| 404 | 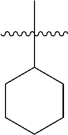 [SMILES: *C1CCCCC1] | H | 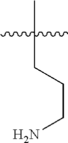 [SMILES: *CCCN] | 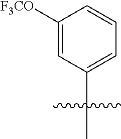 [SMILES: *c1cccc(OC(F)(F)F)c1] | H |
| 420 | 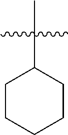 [SMILES: *C1CCCCC1] | H | 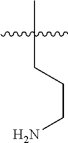 [SMILES: *CCCN] | 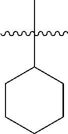 [SMILES: *C1CCCCC1] | H |
| 397 | 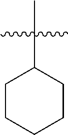 [SMILES: *C1CCCCC1] | H | 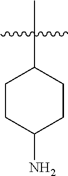 [SMILES: *C1CCC(N)CC1] | 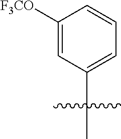 [SMILES: *c1cccc(OC(F)(F)F)c1] | H |
| 436 | 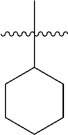 [SMILES: *C1CCCCC1] | H | 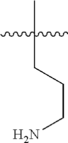 [SMILES: *CCCN] | Br | H |

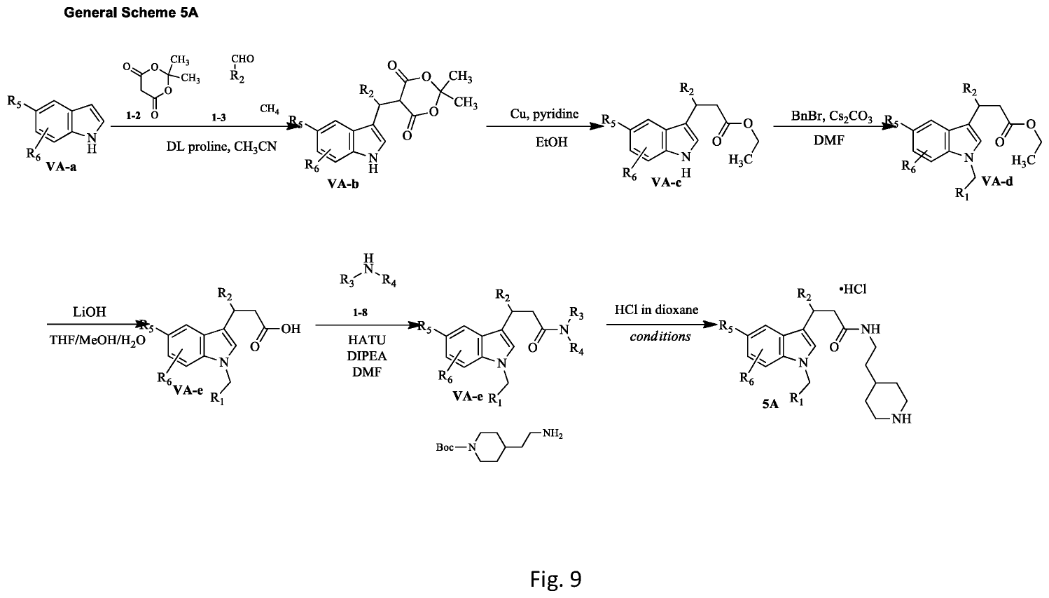

Synthetic route for the synthesis of compound V is described in general scheme 5A (FIG. 9). Condensation reaction with R₂CHO and cyclic ester with indole derivative gave VA-b, which under decarboxylation Cu—EtOH yielded ester derivative VA-d. Saponification of ester and coupling with amine gave amide derivative VA-f. If compound VA-f contains any protecting group as VA-g then final compound V was obtained by deprotection under acidic condition to give acidic salt of free base.

### Example 5A: Synthesis of 3-(1-benzyl-1H-indol-3-yl)-N-(2-(piperidin-4-yl) ethyl)-3-(m-tolyl) propanamide.hydrochloride

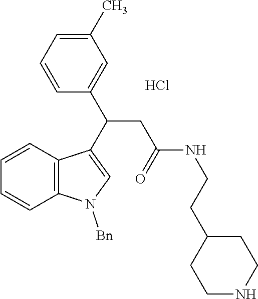 [SMILES: Cc1cccc(C(CC(=O)NCCC2CCNCC2)c2cn(Cc3ccccc3)c3ccccc23)c1 ; Cl]

### Synthesis of 5-((1H-indol-3-yl) (m-tolyl)methyl)-2,2-dimethyl-1,3-dioxane-4,6-dione

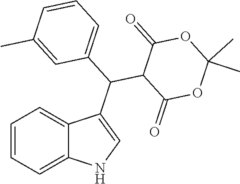

A mixture of indole (2.0 g, 17.1 mmol), Meldrum's acid (3.03 g, 21.0 mmol), m-tolualdehyde (4.1 g 34.2 mmol and DL-proline (100 mg) in CH₃CN (25 mL) were stirred at room temperature for 16 h. The reaction mixture was concentrated under vacuum, and the crude product was carried forward to next step without purification.

### Synthesis of Ethyl 3-(H-indol-3-yl)-3-(m-tolyl)propanoate

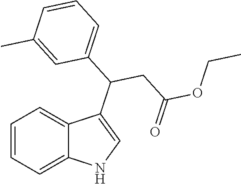 [SMILES: CCOC(=O)CC(c1cccc(C)c1)c1c[nH]c2ccccc12]

To the crude product (4.6 g, 12.6 mmol) in a 1:1 mixture of pyridine and EtOH (60 mL) from previous step Cu powder (80 mg, 1.26 mmol) was added. The reaction mixture was heated to reflux for 16 h. The reaction mixture was filtered and the filtrate was concentrated under vacuum. The residue was purified by column chromatography (silica gel, ethyl acetate/hexanes) to afford as red color oil (2.15 g, 54%). ESI MS m/z 308 [M+H]⁺.

### Synthesis of Ethyl 3-(1-benzyl-1H-indol-3-yl)-3-(m-tolyl) propanoate

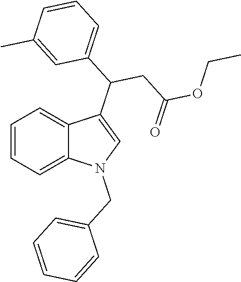 [SMILES: CCOC(=O)CC(c1cccc(C)c1)c1cn(Cc2ccccc2)c2ccccc12]

To a mixture of (1.0 g, 3.45 mmol) and CS₂CO₃ (1.70 g, 5.18 mmol) in DMF (10 mL), benzyl bromide (0.5 mL, 3.80 mmol) was added at 0° C. The reaction mixture was stirred at room temperature for 16 h. The reaction was quenched by the addition of ice water (10 mL) followed by extraction with EtOAc (2×25 mL). The organic layers are recombined, dried over anhydrous MgSO₄ and concentrated under reduced pressure and the crude material was purified by column chromatography (silica gel, EtOAc/Hexanes) to provide intermediate (320 mg, 32%) as a yellow oil. ESI MS m/z 398 [M+H]⁺.

### Synthesis of 3-(1-benzyl-1H-indol-3-yl)-3-(m-tolyl) propanoic Acid

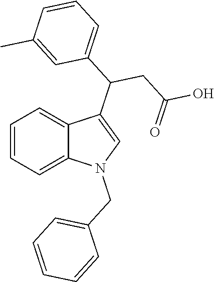 [SMILES: Cc1cccc(C(CC(=O)O)c2cn(Cc3ccccc3)c3ccccc23)c1]

To a solution of (320 mg 0.8 mmol) in mixture of THF/MeOH/H₂O (6 mL), LiGH (192 mg, 8 mmol) was added. The reaction mixture was stirred at room temperature for 8 h and concentrated under vacuum. The residue was dissolved in H₂O (5 mL) and the pH was adjusted to 6.0 using 1N HCl and the aqueous layer was extracted with EtOAc (2×20 mL). The organic layers are recombined, dried over anhydrous MgSO₄ and concentrated under reduced pressure to provide intermediate (254 mg, 85%) as an off white solid. ESI MS m/z 370 [M+H]⁺.

### Synthesis of Tert-Butyl 4-(2-(3-(1-benzyl-1H-indol-3-yl)-3-(m-tolyl) propanamido)-ethyl) piperidine-1-carboxylate

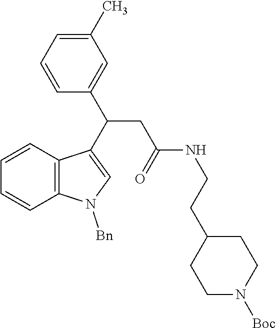 [SMILES: Cc1cccc(C(CC(=O)NCCC2CCN(C(=O)OC(C)(C)C)CC2)c2cn(Cc3ccccc3)c3ccccc23)c1]

To a mixture of (48 mg, 0.13 mmol) in DMF (1.5 mL, HATU (69 mg, 0.18 mmol), DIPEA (45 uL, 0.26 mmol) and) tert-butyl 4-(2-aminoethyl) piperidine-1-carboxylate (35 mg. 0.15 mmol) were added. The reaction mixture was stirred at room temperature for 16 h and was purified by reverse phase column chromatography to afford intermediate (33 mg, 44%) as a white solid. ESI MS m/z 580 [M+H]⁺.

### Synthesis of 3-(1-benzyl-1H-indol-3-yl)-N-(2-(piperidin-4-yl) ethyl)-3-(m-tolyl) propanamide.hydrochloride

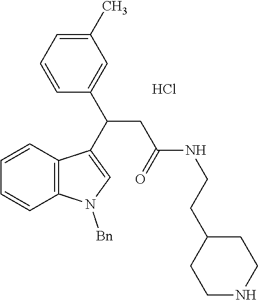 [SMILES: Cc1cccc(C(CC(=O)NCCC2CCNCC2)c2cn(Cc3ccccc3)c3ccccc23)c1 ; Cl]

To a solution of (30 mg, 0.052 mmol) in MeOH (2 mL), HCl in dioxane (4 M, 1 mL) was added. The reaction mixture was stirred at room temperature for 2 h. The reaction mixture was concentrated under vacuum and the residue was lyophilized to afford product (25 mg, 70%) as a brown-red semisolid. ¹H NMR (400 MHz, DMSO-d₆) 8.49 (bs, 1H), 8.21 (bs, 1H), 7.81 (t, J=5.74 Hz, 1H), 7.43 (bs, 1H), 7.36 (d, J=8.61 Hz, 2H), 7.32-7.23 (m, 3H), 7.19-7.15 (m, 2H), 7.13-7.08 (m, 3H), 7.03 (t, J=7.76 Hz, 1H), 6.95-6.88 (m, 2H), 5.37 (bs, 2H), 4.64 (t, J=7.98 Hz, 1H), 3.19-2.98 (m, 4H), 2.95-2.83 (m, 2H), 2.74 (dd, J=14.0, 8.10 Hz, 1H), 2.61-2.55 (m, 1H), 2.23 (bs, 3H), 1.67-1.55 (m, 2H), 1.20-1.08 (m, 5H); HPLC (Method 6) 96.4% (AUC), t_{R}=19.83 min, ESI MS m/z 480 [M+H]⁺.

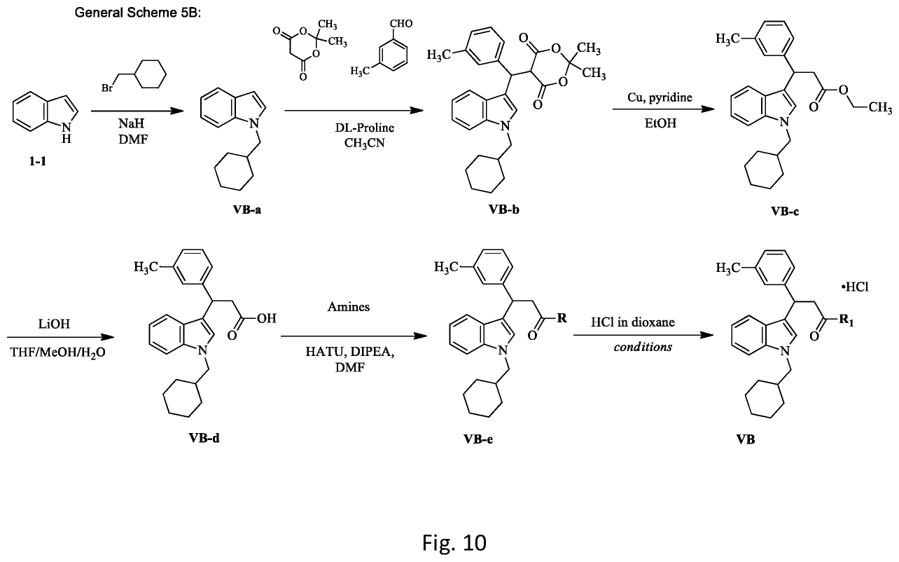

Synthetic route for the synthesis of compound VB is described in general scheme 5B (FIG. 10). N-alkylation of indole with suitable alkyl halide gave compound VB-a, which on condensation with Meldru's acid and proper aldehyde gave compound VB-b followed by decarboxylation under Cu—EtOH yielded ester derivative VB-c. Saponification of ester and coupling with amine gave amide derivative VB-e. If compound VA-f contains any protecting group final compound V was obtained by deprotection under acidic condition to give acidic salt of free base.

### Example 5B: Synthesis of N-((1R,4R)-4-aminocyclohexyl)-3-(1-(cyclohexylmethyl)-1H-indol-3-yl)-3-(m-tolyl)propanamide

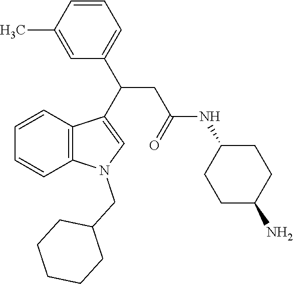 [SMILES: Cc1cccc(C(CC(=O)N[C@H]2CC[C@H](N)CC2)c2cn(CC3CCCCC3)c3ccccc23)c1]

### Synthesis of 1-(cyclohexylmethyl)-1H-indole

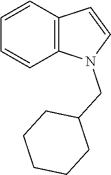 [SMILES: c1ccc2c(c1)ccn2CC1CCCCC1]

To a slurry of NaH (2.0 g, 0.51 mmol) in DMF (25 mL), indole (4.0 g, 34.0 mmol) was added at 0° C. (bromomethyl)cyclohexane (9.8 g, 0.51 mmol) was added and the reaction mixture was stirred at room temperature for 16 h. The reaction was quenched by the addition of water (15 mL) and then extracted with EtOAc (2×30 mL). The EtOAc layer dried (Na₂SO₄), concentrated and the residue was purified by column chromatography (silica gel, EtOAc/Hexanes) to provide 1-(cyclohexylmethyl)-1H-indole as a white sticky solid (6.3 g, 86% yield). ESI MS m/z 214 [M+H]⁺.

### Synthesis of 5-((1-(cyclohexylmethyl)-1H-indol-3-yl) (m-tolyl)methyl)-2,2-dimethyl-1,3-dioxane-4,6-dione

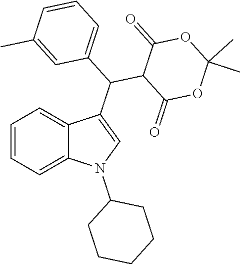

5-((1-(cyclohexylmethyl)-1H-indol-3-yl)(m-tolyl)methyl)-2,2-dimethyl-1,3-dioxane-4,6-dione was prepared by the procedure described for the synthesis of intermediate by stirring a solution of (1.0 equiv), m-tolualdehyde (1.3 equiv), Meldrum's acid (2.0 equiv) and DL-proline (0.1 equiv) in CH₃CN at room temperature for 16 h. The crude 5-((1-(cyclohexylmethyl)-1H-indol-3-yl)(m-tolyl)methyl)-2,2-dimethyl-1,3-dioxane-4,6-dione was carried forward to next step. ESI MS m/z 460 [M+H]⁺.

### Synthesis of Ethyl 3-(1-(cyclohexylmethyl)-1H-indol-3-yl)-3-(m-tolyl) Propanoate

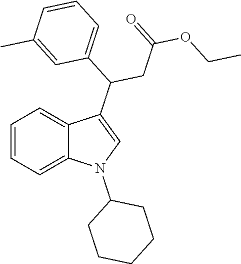 [SMILES: CCOC(=O)CC(c1cccc(C)c1)c1cn(C2CCCCC2)c2ccccc12]

Ethyl 3-(1-(cyclohexylmethyl)-1H-indol-3-yl)-3-(m-tolyl) propanoate was prepared by the procedure described for the synthesis of intermediate 1-5 by heating a solution of 5-((1-(cyclohexylmethyl)-1H-indol-3-yl)(m-tolyl)methyl)-2,2-dimethyl-1,3-dioxane-4,6-dione (1.0 equiv) and Cu powder (0.1 equiv) in a mixture of pyridine/EtOH at 90° C. for 16 h. It was obtained as brown oil (58% yield).

### Synthesis of 3-(1-(cyclohexylmethyl)-1H-indol-3-yl)-3-(m-tolyl) Propanoic Acid

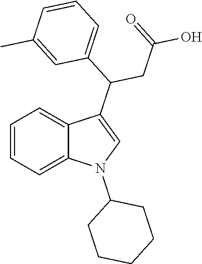 [SMILES: Cc1cccc(C(CC(=O)O)c2cn(C3CCCCC3)c3ccccc23)c1]

3-(1-(cyclohexylmethyl)-1H-indol-3-yl)-3-(m-tolyl)propanoic acid was prepared by the ester hydrolysis of ethyl 3-(1-(cyclohexylmethyl)-1H-indol-3-yl)-3-(m-tolyl)propanoate (1.0 equiv) and LiGH (10.0 equiv) in a mixture of THF/MeOH/H20 (1:1:1) at room temperature for 4-6 h. It was obtained as an off-white solid (90% yield).

General Procedure for the Synthesis of Amide Intermediates:

To a mixture of 8 (1.0 equiv) HATU (1.5 equiv) and DIPEA (2.0 equiv) in DMF (1 mL) the corresponding amines (1.3 equiv) were added. The reaction mixture was stirred at room temperature for 16 h and was purified by either reverse phase C18 column chromatography or by precipitation by addition of water to afford the amide intermediates.

General Procedure for the Deprotection of BOC Group:

The amide intermediates with a Boc group were subjected Boc deprotection by adding HCl in dioxane to a solution of amide intermediates in MeOH. The reaction mixture was then concentrated in vacuo, the residue was washed with solvents such as EtOAc or CH₃CN, followed by lyophilisation. Those intermediates that have basic nitrogen are converted to the corresponding hydrochloride salts by the addition of 1 M HCl to a suspension of the intermediate in H₂O followed by lyophilisation.

### Synthesis of N-((1R,4R)-4-aminocyclohexyl)-3-(1-(cyclohexylmethyl)-1H-indol-3-yl)-3-(m-tolyl) Propanamide

¹H NMR (400 MHz, Methanol-d₄) δ 7.30 (t, J=8.8 Hz, 2H), 7.14-7.04 (m, 5H), 6.96 (t, J=7.5 Hz, 1H), 6.91-6.86 (m, 1H), 4.69 (t, J=8.1 Hz, 1H), 3.96 (d, J=7.2 Hz, 2H), 3.04-2.92 (m, 2H), 2.84-2.75 (m, 1H), 2.25 (s, 3H), 2.00-1.91 (m, 2H), 1.90-1.81 (m, 1H), 1.80-1.65 (m, 5H), 1.64-1.54 (m, 2H), 1.45-1.32 (m, 2H), 1.27-1.17 (m, 4H), 1.16-0.95 (m, 4H; HPLC (Method 5) 93.6% (AUC), t_{R}=12.28 min; ESI-MS m/z 472 [M+H]⁺.

Following the procedure described in scheme 5A & 5B/Example VA & VB, compounds of Table 5 are prepared by varying suitable starting materials and proper conditions.

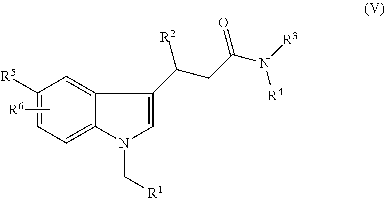

TABLE 5

| Cmpd | R¹ | R² | R³ | R⁴ | R⁵ | R⁶ |
|---|---|---|---|---|---|---|
| 002 | Ph | 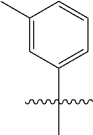 [SMILES: *c1cccc(C)c1] | H | 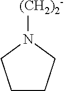 | H | H |
| 045 | Ph | 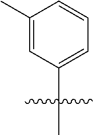 [SMILES: *c1cccc(C)c1] | H | 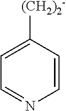 | H | H |
| 045 | Ph | 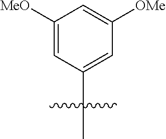 [SMILES: *c1cc(OC)cc(OC)c1] | H | 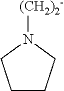 | H | H |
| 047 | 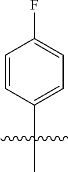 [SMILES: *c1ccc(F)cc1] | 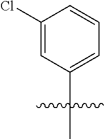 [SMILES: *c1cccc(Cl)c1] | H | 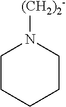 | H | H |
| 049 | 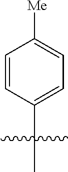 [SMILES: *c1ccc(C)cc1] | 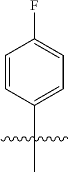 [SMILES: *c1ccc(F)cc1] | H | 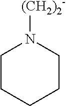 | H | H |
| 050 | 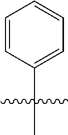 [SMILES: *c1ccccc1] | 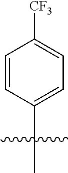 [SMILES: *c1ccc(C(F)(F)F)cc1] | H | 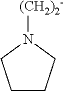 | H | H |
| 051 | Ph | 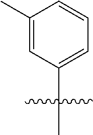 [SMILES: *c1cccc(C)c1] | H | 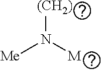 | H | H |
| 064 | Ph | 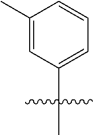 [SMILES: *c1cccc(C)c1] | H | 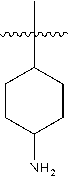 [SMILES: *C1CCC(N)CC1] | H | H |
| 066 | Ph | 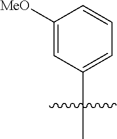 [SMILES: *c1cccc(OC)c1] | H | 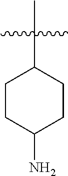 [SMILES: *C1CCC(N)CC1] | H | H |
| 067 | Ph | 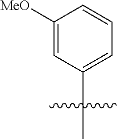 [SMILES: *c1cccc(OC)c1] | H | 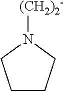 | H | H |
| 068 | Ph | 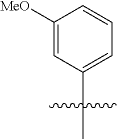 [SMILES: *c1cccc(OC)c1] | H | 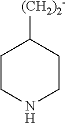 | H | H |
| 072 | Ph | 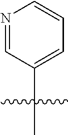 [SMILES: *c1cccnc1] | H | 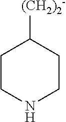 | H | H |
| 073 | Ph | 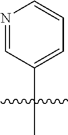 [SMILES: *c1cccnc1] | H | 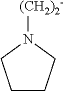 | H | H |
| 052 | Ph | 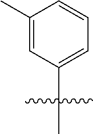 [SMILES: *c1cccc(C)c1] | H | 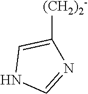 | H | H |
| 053 | Ph | 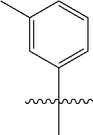 [SMILES: *c1cccc(C)c1] | H | 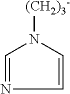 | H | H |
| 054 | Ph | 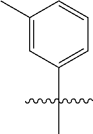 [SMILES: *c1cccc(C)c1] | H | 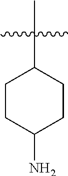 [SMILES: *C1CCC(N)CC1] | H | H |
| 055 | Ph | 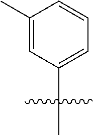 [SMILES: *c1cccc(C)c1] | H | 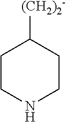 | H | H |
| 058 | Ph | 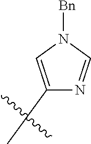 [SMILES: *c1cn(Cc2ccccc2)cn1] | H | 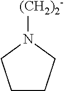 | H | H |
| 059 | Ph | 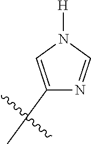 [SMILES: *c1c[nH]cn1] | H | 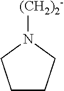 | H | H |
| 060 | Ph | 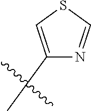 [SMILES: *c1cscn1] | H | 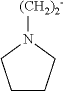 | H | H |
| 078 | Ph | 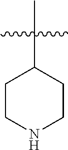 [SMILES: *C1CCNCC1] | H | 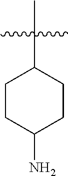 [SMILES: *C1CCC(N)CC1] | H | H |
| 079 | Ph | 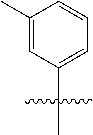 [SMILES: *c1cccc(C)c1] | H | 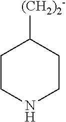 | H | H |
| 080 | Ph | 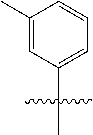 [SMILES: *c1cccc(C)c1] | H | 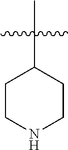 [SMILES: *C1CCNCC1] | H | H |
| 084 | CH₃ | 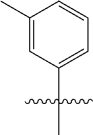 [SMILES: *c1cccc(C)c1] | H | 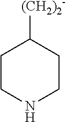 | H | H |
| | Ph | 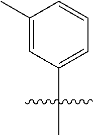 [SMILES: *c1cccc(C)c1] | H | 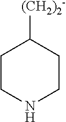 | H | H |
| 074 | Ph | 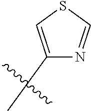 [SMILES: *c1cscn1] | H | 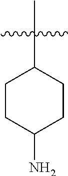 [SMILES: *C1CCC(N)CC1] | H | H |
| 075 | Ph | 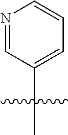 [SMILES: *c1cccnc1] | H | 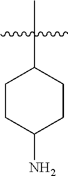 [SMILES: *C1CCC(N)CC1] | H | H |
| 076 | Ph | 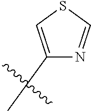 [SMILES: *c1cscn1] | H | 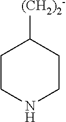 | H | H |
| 077 | Ph | 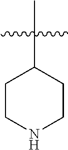 [SMILES: *C1CCNCC1] | H | 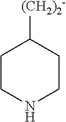 | H | H |
| 091 | 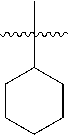 [SMILES: *C1CCCCC1] | 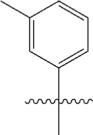 [SMILES: *c1cccc(C)c1] | H | 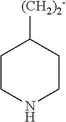 | H | H |
| 73 | Ph | 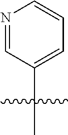 [SMILES: *c1cccnc1] | H | 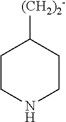 | H | H |
| 95 | Ph | 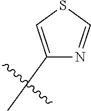 [SMILES: *c1cscn1] | H | 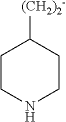 | H | H |
| 96 | Ph | 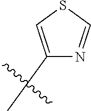 [SMILES: *c1cscn1] | H | 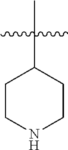 [SMILES: *C1CCNCC1] | H | H |
| 099 | Ph | 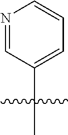 [SMILES: *c1cccnc1] | H | 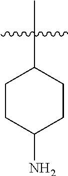 [SMILES: *C1CCC(N)CC1] | H | H |
| 100 | Ph | H | H | 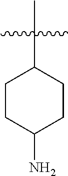 [SMILES: *C1CCC(N)CC1] | H | H |
| 101 | 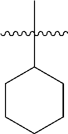 [SMILES: *C1CCCCC1] | 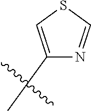 [SMILES: *c1cscn1] | H | 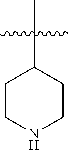 [SMILES: *C1CCNCC1] | H | H |
| 102 | Ph | H | H | 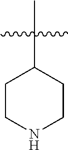 [SMILES: *C1CCNCC1] | H | H |
| 088 | Ph | H | H | 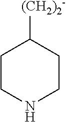 | H | H |
| 089 | 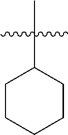 [SMILES: *C1CCCCC1] | 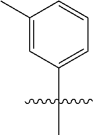 [SMILES: *c1cccc(C)c1] | H | 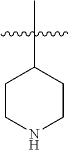 [SMILES: *C1CCNCC1] | H | H |
| 091 | CH₃ | 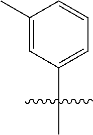 [SMILES: *c1cccc(C)c1] | H | 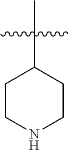 [SMILES: *C1CCNCC1] | H | H |
| 104 | 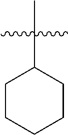 [SMILES: *C1CCCCC1] | 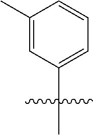 [SMILES: *c1cccc(C)c1] | H | 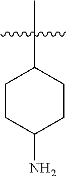 [SMILES: *C1CCC(N)CC1] | H | H |
| 106 | 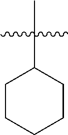 [SMILES: *C1CCCCC1] | 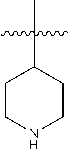 [SMILES: *C1CCNCC1] | H | 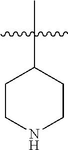 [SMILES: *C1CCNCC1] | H | H |
| 107 | 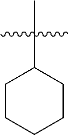 [SMILES: *C1CCCCC1] | 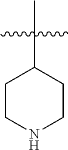 [SMILES: *C1CCNCC1] | H | 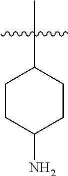 [SMILES: *C1CCC(N)CC1] | H | H |
| 108 | 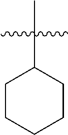 [SMILES: *C1CCCCC1] | 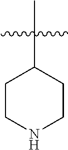 [SMILES: *C1CCNCC1] | H | 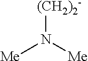 | H | H |
| 109 | 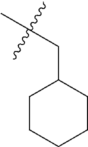 [SMILES: *CC1CCCCC1] | 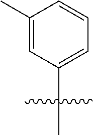 [SMILES: *c1cccc(C)c1] | H | 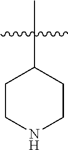 [SMILES: *C1CCNCC1] | H | H |
| 110 | 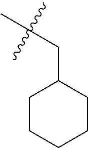 [SMILES: *CC1CCCCC1] | 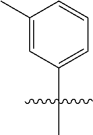 [SMILES: *c1cccc(C)c1] | H | 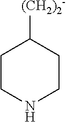 | H | H |
| 111 | 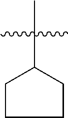 [SMILES: *C1CCCC1] | 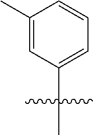 [SMILES: *c1cccc(C)c1] | H | 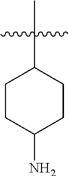 [SMILES: *C1CCC(N)CC1] | H | H |
| 112 | 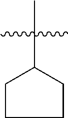 [SMILES: *C1CCCC1] | 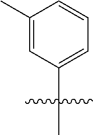 [SMILES: *c1cccc(C)c1] | H | 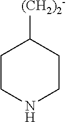 | H | H |
| 113 | 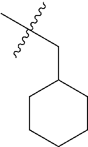 [SMILES: *CC1CCCCC1] | 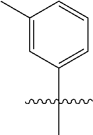 [SMILES: *c1cccc(C)c1] | H | 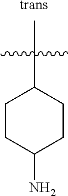 [SMILES: "trans" ; *C1CCC(N)CC1] | H | H |
| 103 | 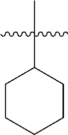 [SMILES: *C1CCCCC1] | 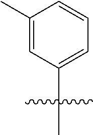 [SMILES: *c1cccc(C)c1] | H | 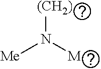 | H | H |
| 114 | 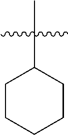 [SMILES: *C1CCCCC1] | 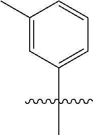 [SMILES: *c1cccc(C)c1] | H | 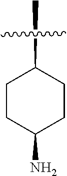 | H | H |
| 115 | 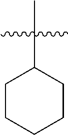 [SMILES: *C1CCCCC1] | H | H | 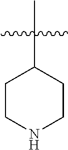 [SMILES: *C1CCNCC1] | H | H |
| 116 | 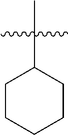 [SMILES: *C1CCCCC1] | H | H | 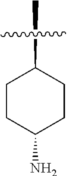 | H | H |
| 117 | 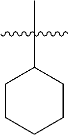 [SMILES: *C1CCCCC1] | H | H | 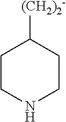 | H | H |
| 118 | 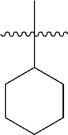 [SMILES: *C1CCCCC1] | 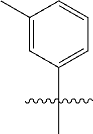 [SMILES: *c1cccc(C)c1] | H | 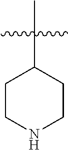 [SMILES: *C1CCNCC1] | H | H |
| 119 | 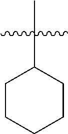 [SMILES: *C1CCCCC1] | 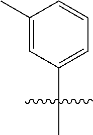 [SMILES: *c1cccc(C)c1] | H | 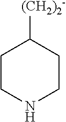 | H | H |
| 120 | 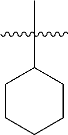 [SMILES: *C1CCCCC1] | 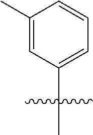 [SMILES: *c1cccc(C)c1] | H | 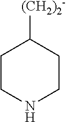 | Br | H |
| 121 | 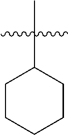 [SMILES: *C1CCCCC1] | 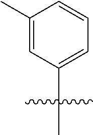 [SMILES: *c1cccc(C)c1] | H | 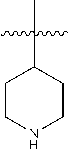 [SMILES: *C1CCNCC1] | Br | H |
| 132 | 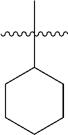 [SMILES: *C1CCCCC1] | 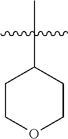 [SMILES: *C1CCOCC1] | H | 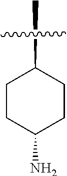 | H | H |
| 133 | 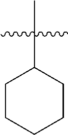 [SMILES: *C1CCCCC1] | 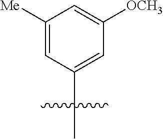 [SMILES: *c1cc(C)cc(OC)c1] | H | 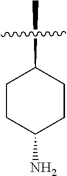 | H | H |
| 123 | 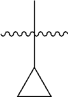 [SMILES: *C1CC1] | 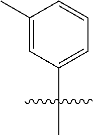 [SMILES: *c1cccc(C)c1] | H | 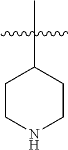 [SMILES: *C1CCNCC1] | H | H |
| 124 | 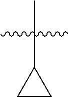 [SMILES: *C1CC1] | 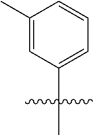 [SMILES: *c1cccc(C)c1] | H | 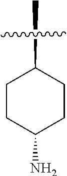 | H | H |
| 125 | 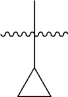 [SMILES: *C1CC1] | 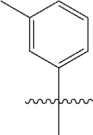 [SMILES: *c1cccc(C)c1] | H | 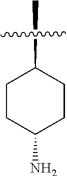 | F | H |
| 126 | 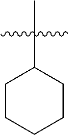 [SMILES: *C1CCCCC1] | 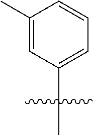 [SMILES: *c1cccc(C)c1] | H | 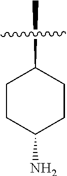 | F | H |
| 127 | 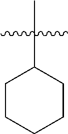 [SMILES: *C1CCCCC1] | 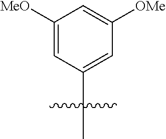 [SMILES: *c1cc(OC)cc(OC)c1] | H | 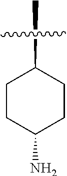 | H | H |
| 128 | 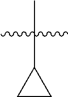 [SMILES: *C1CC1] | 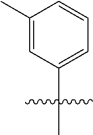 [SMILES: *c1cccc(C)c1] | H | 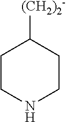 | H | H |
| | 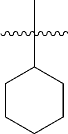 [SMILES: *C1CCCCC1] | 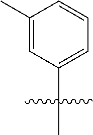 [SMILES: *c1cccc(C)c1] | H | 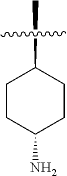 | H | H |
| 130 | 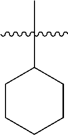 [SMILES: *C1CCCCC1] | 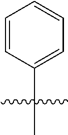 [SMILES: *c1ccccc1] | H | 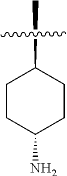 | H | H |
| 131 | 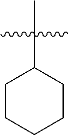 [SMILES: *C1CCCCC1] | 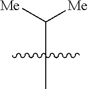 [SMILES: *C(C)C] | H | 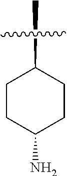 | H | H |
| 142 | 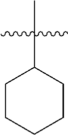 [SMILES: *C1CCCCC1] | 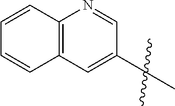 [SMILES: *c1cnc2ccccc2c1] | H | 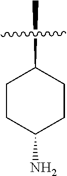 | H | H |
| 143 | 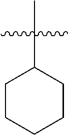 [SMILES: *C1CCCCC1] | 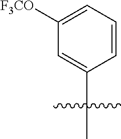 [SMILES: *c1cccc(OC(F)(F)F)c1] | H | 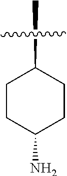 | H | H |
| 134 | 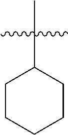 [SMILES: *C1CCCCC1] | 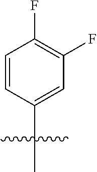 [SMILES: *c1ccc(F)c(F)c1] | H | 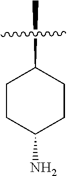 | H | H |
| 135 | 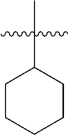 [SMILES: *C1CCCCC1] | 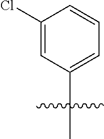 [SMILES: *c1cccc(Cl)c1] | H | 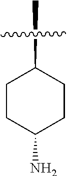 | H | H |
| 136 | 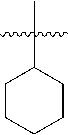 [SMILES: *C1CCCCC1] | 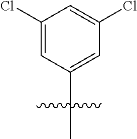 [SMILES: *c1cc(Cl)cc(Cl)c1] | H | 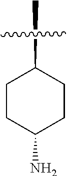 | H | H |
| 137 | 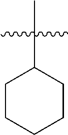 [SMILES: *C1CCCCC1] | 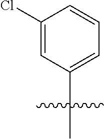 [SMILES: *c1cccc(Cl)c1] | H | 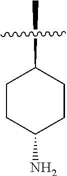 | H | H |
| 138 | 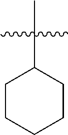 [SMILES: *C1CCCCC1] | 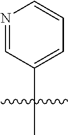 [SMILES: *c1cccnc1] | H | 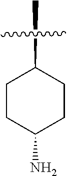 | H | H |
| 139 | 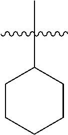 [SMILES: *C1CCCCC1] | 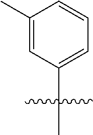 [SMILES: *c1cccc(C)c1] | H | 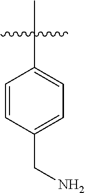 [SMILES: *c1ccc(CN)cc1] | H | H |
| 140 | 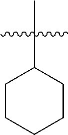 [SMILES: *C1CCCCC1] | 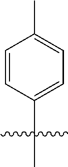 [SMILES: *c1ccc(C)cc1] | H | 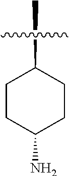 | H | H |
| 156 | 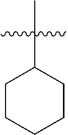 [SMILES: *C1CCCCC1] | H | H | 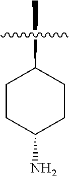 | Br | H |
| 157 | 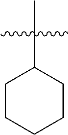 [SMILES: *C1CCCCC1] | 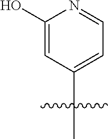 [SMILES: *c1ccnc(O)c1] | H | 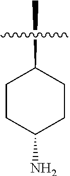 | H | H |
| 158 | 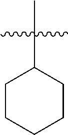 [SMILES: *C1CCCCC1] | 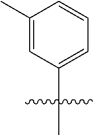 [SMILES: *c1cccc(C)c1] | H | 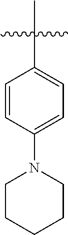 [SMILES: *c1ccc(N2CCCCC2)cc1] | H | H |
| 159 | 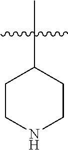 [SMILES: *C1CCNCC1] | 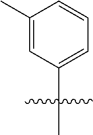 [SMILES: *c1cccc(C)c1] | H | 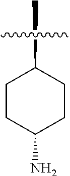 | H | H |
| 145 | 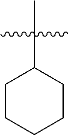 [SMILES: *C1CCCCC1] | 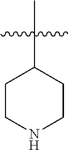 [SMILES: *C1CCNCC1] | H | 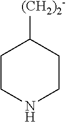 | H | H |
| 146 | 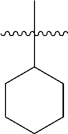 [SMILES: *C1CCCCC1] | 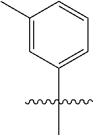 [SMILES: *c1cccc(C)c1] | H | 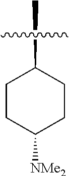 | H | H |
| | 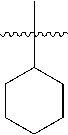 [SMILES: *C1CCCCC1] | 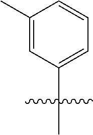 [SMILES: *c1cccc(C)c1] | H | 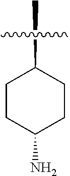 | H | H |
| 150 | 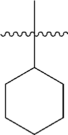 [SMILES: *C1CCCCC1] | 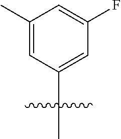 [SMILES: *c1cc(C)cc(F)c1] | H | 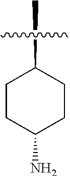 | H | H |
| 151 | 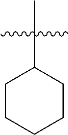 [SMILES: *C1CCCCC1] | 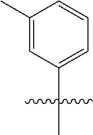 [SMILES: *c1cccc(C)c1] | H | 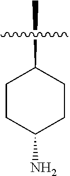 | OCH₃ | H |
| 154 | 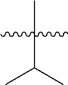 [SMILES: *C(C)C] | 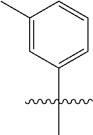 [SMILES: *c1cccc(C)c1] | H | 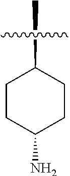 | H | H |
| 155 | 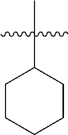 [SMILES: *C1CCCCC1] | 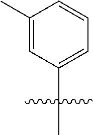 [SMILES: *c1cccc(C)c1] | H | 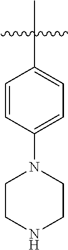 [SMILES: *c1ccc(N2CCNCC2)cc1] | H | H |
| 167 | 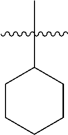 [SMILES: *C1CCCCC1] | 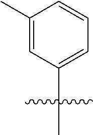 [SMILES: *c1cccc(C)c1] | 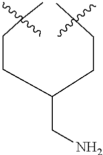 [SMILES: *CCC(CN)CC*] | | H | H |
| 174 | 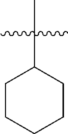 [SMILES: *C1CCCCC1] | 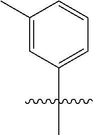 [SMILES: *c1cccc(C)c1] | H | 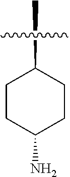 | H | 8-Br |
| 175 | 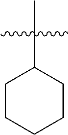 [SMILES: *C1CCCCC1] | 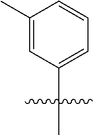 [SMILES: *c1cccc(C)c1] | H | 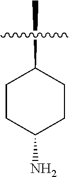 | H | 7-Br |
| 178 | 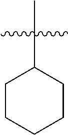 [SMILES: *C1CCCCC1] | 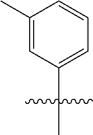 [SMILES: *c1cccc(C)c1] | H | 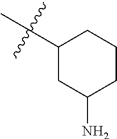 [SMILES: *C1CCCC(N)C1] | H | H |
| 160 | 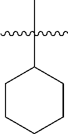 [SMILES: *C1CCCCC1] | 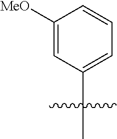 [SMILES: *c1cccc(OC)c1] | H | 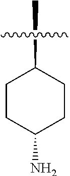 | H | H |
| 161 | 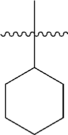 [SMILES: *C1CCCCC1] | 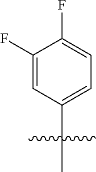 [SMILES: *c1ccc(F)c(F)c1] | H | 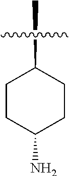 | H | H |
| 162 | 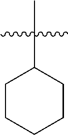 [SMILES: *C1CCCCC1] | 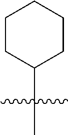 [SMILES: *C1CCCCC1] | H | 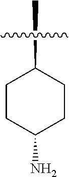 | H | H |
| 165 | 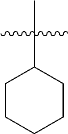 [SMILES: *C1CCCCC1] | 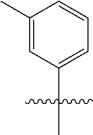 [SMILES: *c1cccc(C)c1] | H | 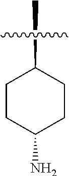 | OH | H |
| 166 | 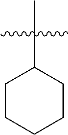 [SMILES: *C1CCCCC1] | 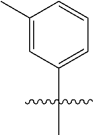 [SMILES: *c1cccc(C)c1] | H | 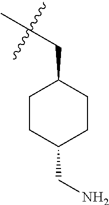 [SMILES: *C[C@H]1CC[C@H](CN)CC1] | H | H |
| 189 | 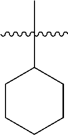 [SMILES: *C1CCCCC1] | 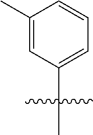 [SMILES: *c1cccc(C)c1] | H | 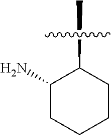 | H | H |
| 198 | 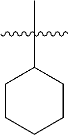 [SMILES: *C1CCCCC1] | 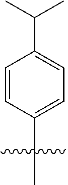 [SMILES: *c1ccc(C(C)C)cc1] | H | 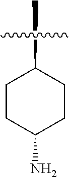 | H | H |
| 201 | 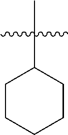 [SMILES: *C1CCCCC1] | 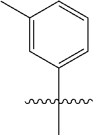 [SMILES: *c1cccc(C)c1] | H | 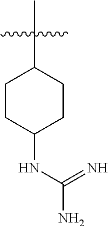 [SMILES: *C1CCC(NC(=N)N)CC1] | H | H |
| 202 | 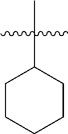 [SMILES: *C1CCCCC1] | 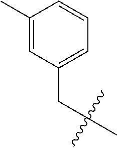 [SMILES: *Cc1cccc(C)c1] | H | 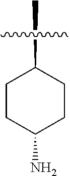 | H | H |
| 203 | 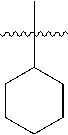 [SMILES: *C1CCCCC1] | 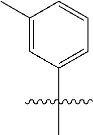 [SMILES: *c1cccc(C)c1] | H | 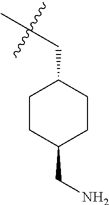 [SMILES: *C[C@H]1CC[C@H](CN)CC1] | Br | H |
| 179 | 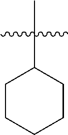 [SMILES: *C1CCCCC1] | 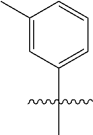 [SMILES: *c1cccc(C)c1] | 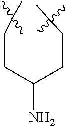 [SMILES: *CCC(N)CC*] | | H | H |
| 180 | 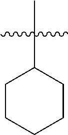 [SMILES: *C1CCCCC1] | 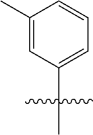 [SMILES: *c1cccc(C)c1] | H | 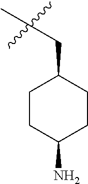 [SMILES: *C[C@H]1CC[C@@H](N)CC1] | H | H |
| 181 | 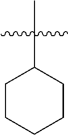 [SMILES: *C1CCCCC1] | 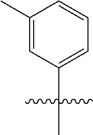 [SMILES: *c1cccc(C)c1] | 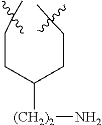 | | H | H |
| 182 | 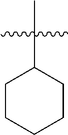 [SMILES: *C1CCCCC1] | 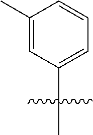 [SMILES: *c1cccc(C)c1] | H | 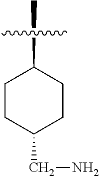 | H | H |
| 210 | 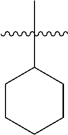 [SMILES: *C1CCCCC1] | 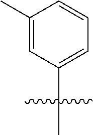 [SMILES: *c1cccc(C)c1] | H | 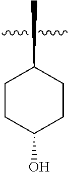 | H | H |
| 214 | 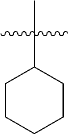 [SMILES: *C1CCCCC1] | 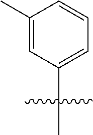 [SMILES: *c1cccc(C)c1] | H | 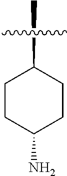 | Br | H |
| 223 | Adamantyl | 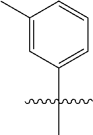 [SMILES: *c1cccc(C)c1] | H | 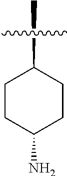 | H | H |
| 226 | 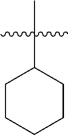 [SMILES: *C1CCCCC1] | 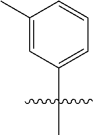 [SMILES: *c1cccc(C)c1] | H | 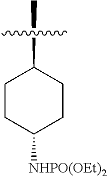 | H | H |
| 227 | 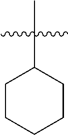 [SMILES: *C1CCCCC1] | 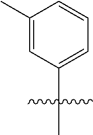 [SMILES: *c1cccc(C)c1] | | 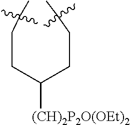 | H | H |
| 236 | 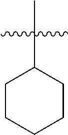 [SMILES: *C1CCCCC1] | 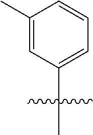 [SMILES: *c1cccc(C)c1] | H | 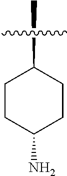 | CN | H |
| 206 | 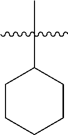 [SMILES: *C1CCCCC1] | 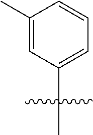 [SMILES: *c1cccc(C)c1] | H | 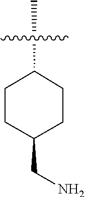 | Br | H |
| 208 | 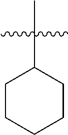 [SMILES: *C1CCCCC1] | 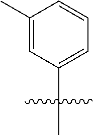 [SMILES: *c1cccc(C)c1] | H | 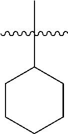 [SMILES: *C1CCCCC1] | H | H |
| 247 | 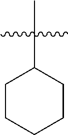 [SMILES: *C1CCCCC1] | 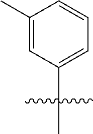 [SMILES: *c1cccc(C)c1] | H | 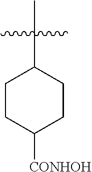 | H | H |
| 259 | 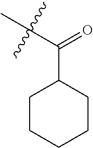 [SMILES: *C(=O)C1CCCCC1] | 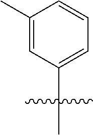 [SMILES: *c1cccc(C)c1] | H | 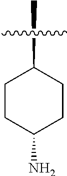 | H | H |
| 264 | 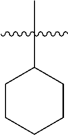 [SMILES: *C1CCCCC1] | H; R10 = NH2 | H | 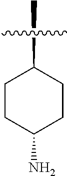 | H | H |
| 270 | 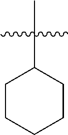 [SMILES: *C1CCCCC1] | H | H | 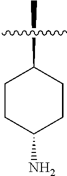 | Br | H |
| 278 | 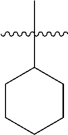 [SMILES: *C1CCCCC1] | 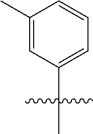 [SMILES: *c1cccc(C)c1] | Me | 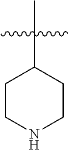 [SMILES: *C1CCNCC1] | Br | H |
| 279 | 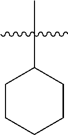 [SMILES: *C1CCCCC1] | 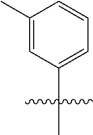 [SMILES: *c1cccc(C)c1] | Me | 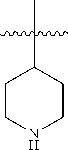 [SMILES: *C1CCNCC1] | H | H |
| 208 | 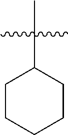 [SMILES: *C1CCCCC1] | 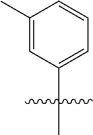 [SMILES: *c1cccc(C)c1] | H | 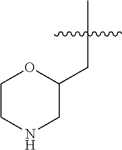 [SMILES: *CC1CNCCO1] | H | H |
| 281 | 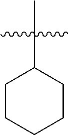 [SMILES: *C1CCCCC1] | 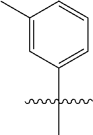 [SMILES: *c1cccc(C)c1] | H | 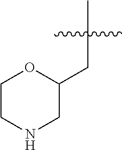 [SMILES: *CC1CNCCO1] | Br | H |
| 238 | 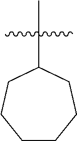 [SMILES: *C1CCCCCC1] | 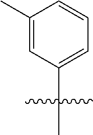 [SMILES: *c1cccc(C)c1] | H | 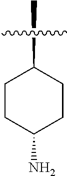 | H | H |
| 243 | | | | | | |
| 282 | 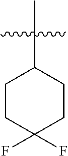 [SMILES: *C1CCC(F)(F)CC1] | 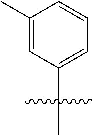 [SMILES: *c1cccc(C)c1] | H | 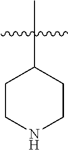 [SMILES: *C1CCNCC1] | H | H |
| 284 | 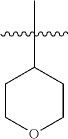 [SMILES: *C1CCOCC1] | 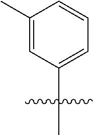 [SMILES: *c1cccc(C)c1] | H | 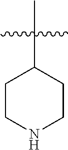 [SMILES: *C1CCNCC1] | H | H |
| 285 | 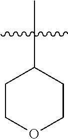 [SMILES: *C1CCOCC1] | 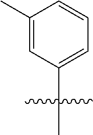 [SMILES: *c1cccc(C)c1] | H | 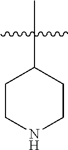 [SMILES: *C1CCNCC1] | Br | H |
| 286 | 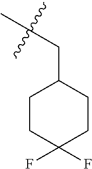 [SMILES: *CC1CCC(F)(F)CC1] | H | H | 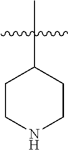 [SMILES: *C1CCNCC1] | Br | H |
| 287 | 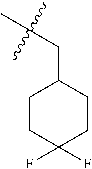 [SMILES: *CC1CCC(F)(F)CC1] | 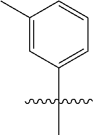 [SMILES: *c1cccc(C)c1] | H | 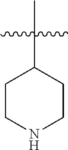 [SMILES: *C1CCNCC1] | Br | H |
| 296 | 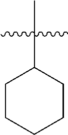 [SMILES: *C1CCCCC1] | 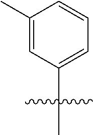 [SMILES: *c1cccc(C)c1] | H | 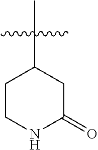 [SMILES: *C1CCNC(=O)C1] | Br | H |
| 297 | 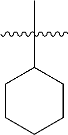 [SMILES: *C1CCCCC1] | 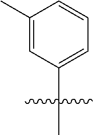 [SMILES: *c1cccc(C)c1] | H | 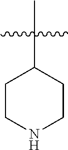 [SMILES: *C1CCNCC1] | COOH | H |
| 298 | 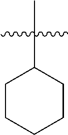 [SMILES: *C1CCCCC1] | H | H | 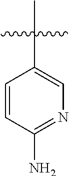 [SMILES: *c1ccc(N)nc1] | Br | H |
| 299 | 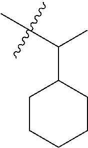 [SMILES: *C(C)C1CCCCC1] | H | H | 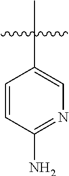 [SMILES: *c1ccc(N)nc1] | Br | H |

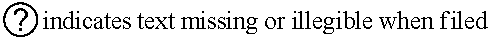

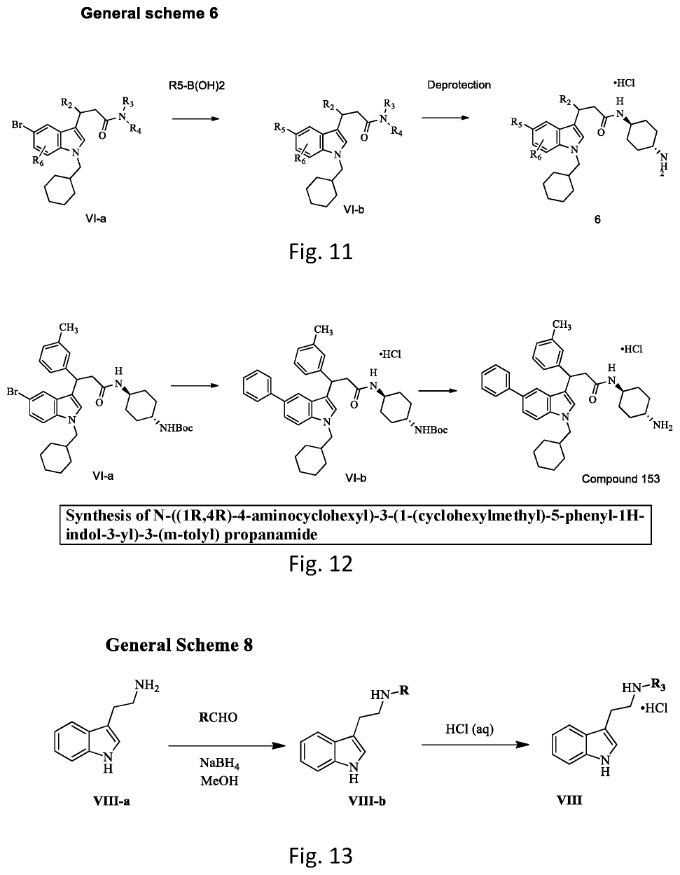

Compound VI-a (General Scheme 6, FIG. 11) was synthesized following the process followed in scheme 5B starting with 5-Br indole, followed by coupling with suitable boronic acid and followed deprotection gave compound VI.

### Example VI: Synthesis of N-((1R,4R)-4-aminocyclohexyl)-3-(1-(cyclohexylmethyl)-5-phenyl-1H-indol-3-yl)-3-(m-tolyl) propanamide

See FIG. 12.

Pd(PPh₃)₄ (5.3 mg, 0.0046 mmol), sodium carbonate (14.49 mg, 0.138 mmol), phenylboronic acid (6.67, 0.552 mmol) and tert-butyl ((1R,4R)-4-(3-(5-bromo-1-(cyclohexylmethyl)-1H-indol-3-yl)-3-(m-tolyl)propanamido)cyclohexyl)carbamate (30 mg, 0.046 mmol) were added to the 2 mL of degassed mixture of 1,4-dioxane and water (8:2). Reaction was heated in a microwave oven for 1 h at 120° C. The reaction mixture was diluted with EtOAc (25 mL) and washed with H₂O (30 ml×2). The EtOAc layer was dried (Na₂SO₄), concentrated in vacuo and the residue was purified by combi-flash chromatography (silica gel, Ethyl acetate/hexanes) to afford tert-butyl ((1R,4R)-4-(3-(1-(cyclohexylmethyl)-5-phenyl-1H-indol-3-yl)-3-(m-tolyl)propanamido)cyclohexyl)carbamate (17 mg, 33%) as a white solid. APCI MS m/z 648 [M+H]+. Which was deprotected under acidic condition to obtain title compound.

¹H NMR (400 MHz, DMSO-d₆) δ 7.80 (bs, 4H), 7.55-7.50 (m, 3H), 7.47 (d, J=7.4 Hz, 1H), 7.41 (t, J=7.4 Hz, 2H), 7.37-7.32 (m, 1H), 7.30-7.23 (m, 2H), 7.14-7.03 (m, 3H), 6.94-6.87 (m, 1H), 4.68 (t, J=7.9 Hz, 1H), 3.98 (d, J=7.2 Hz, 2H), 3.70-3.53 (m, 1H), 3.40-3.32 (m, 1H), 3.16-3.06 (m, 1H), 2.99-2.79 (m, 2H), 2.76-2.61 (m, 1H), 2.21 (s, 3H), 1.89-1.81 (m, 2H), 1.69-1.60 (m, 4H), 1.56-1.50 (m, 1H), 1.30-1.23 (m, 7H), 1.17-1.07 (m, 3H). HPLC (Method 5) 98.1% (AUC), t_{R}=13.31 min; ESI-MS m/z 548.6 [M+H]⁺.

Following the procedure described in scheme 6/Example VI, compounds of Table 6 are prepared by using suitable starting materials and proper conditions.

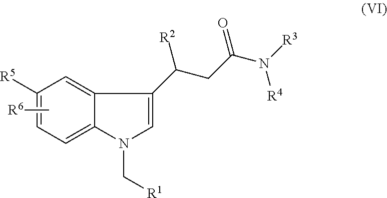

TABLE 6

| Cmpd | R¹ | R² | R³ | R⁴ | R⁵ | R⁶ |
|---|---|---|---|---|---|---|
| 144 | 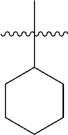 [SMILES: *C1CCCCC1] | H | H | 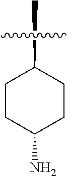 | 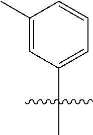 [SMILES: *c1cccc(C)c1] | H |
| 219 | 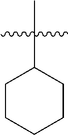 [SMILES: *C1CCCCC1] | 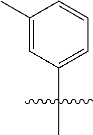 [SMILES: *c1cccc(C)c1] | H | 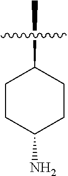 | 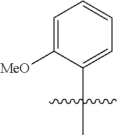 [SMILES: *c1ccccc1OC] | H |
| 153 | 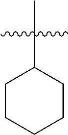 [SMILES: *C1CCCCC1] | 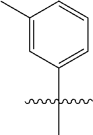 [SMILES: *c1cccc(C)c1] | H | 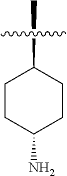 | 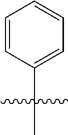 [SMILES: *c1ccccc1] | H |
| 183 | 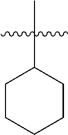 [SMILES: *C1CCCCC1] | 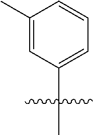 [SMILES: *c1cccc(C)c1] | H | 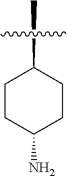 | 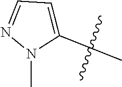 [SMILES: *c1ccnn1C] | H |
| 188 | 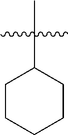 [SMILES: *C1CCCCC1] | 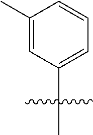 [SMILES: *c1cccc(C)c1] | H | 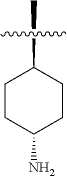 | 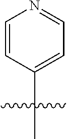 [SMILES: *c1ccncc1] | H |
| 205 | 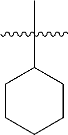 [SMILES: *C1CCCCC1] | 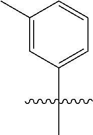 [SMILES: *c1cccc(C)c1] | H | 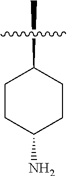 | 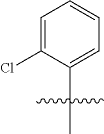 [SMILES: *c1ccccc1Cl] | H |
| 213 | 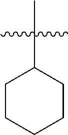 [SMILES: *C1CCCCC1] | H | H | 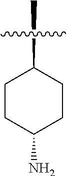 | 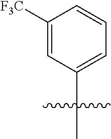 [SMILES: *c1cccc(C(F)(F)F)c1] | H |
| 221 | 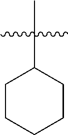 [SMILES: *C1CCCCC1] | 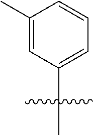 [SMILES: *c1cccc(C)c1] | H | 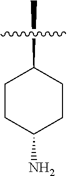 | 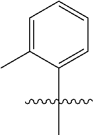 [SMILES: *c1ccccc1C] | H |
| 228 | 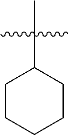 [SMILES: *C1CCCCC1] | 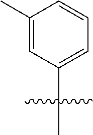 [SMILES: *c1cccc(C)c1] | H | 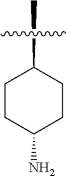 | 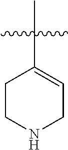 [SMILES: *C1=CCNCC1] | H |
| 230 | 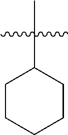 [SMILES: *C1CCCCC1] | H | H | 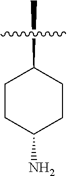 | 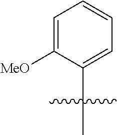 [SMILES: *c1ccccc1OC] | H |
| 231 | 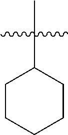 [SMILES: *C1CCCCC1] | H | H | 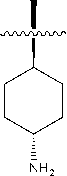 | 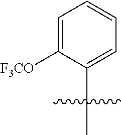 [SMILES: *c1ccccc1OC(F)(F)F] | H |
| 239 | 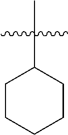 [SMILES: *C1CCCCC1] | H | H | 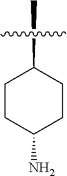 | 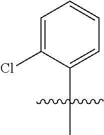 [SMILES: *c1ccccc1Cl] | H |

The general scheme 8 (FIG. 13) illustrates for synthesis of compound VIII. Reductive amination of VIII-a with appropriate aldehyde RCHO gave VIII-b, which under acidic condition undergoes N-deprotection and yields salt of compound VIII.

### Example VIII: Synthesis of 2-(1H-indol-3-yl)-N-(3-phenoxybenzyl) ethan-1-amine

General Procedure for Reductive Amination:

A mixture of tryptamine (1.0 equiv) and the corresponding aldehyde (1.05 equiv) was stirred at room temperature for 1 h. The reaction mixture was then cooled to 0° C. and NaBH₄ (1.2 equiv) was added. The reaction mixture was stirred at room temperature for 2-16 h. Upon completion, the reaction mixture was cooled to 0° C., quenched by dropwise addition of H₂O and extracted with CH₂Cl₂. The CH₂C12 layer was dried (Na₂SO₄), concentrated and the residue was purified by column chromatography (silica gel, EtOAc/Hexanes) to afford intermediates VIII-b.

General Procedure for Boc Deprotection/HCl Salt Formation:

The intermediates with a Boc group were subjected Boc deprotection by adding HCl in dioxane to a solution of intermediates in MeOH. The reaction mixture was then concentrated in vacuo, the residue was washed with solvents such as EtOAc or CH₃CN, followed by lyophilisation. Those intermediates that have a basic nitrogen are converted to the corresponding hydrochloride salts.

Following the procedure described in scheme 8/Example VIII, compounds of Table 8 are prepared by using suitable starting materials and proper conditions.

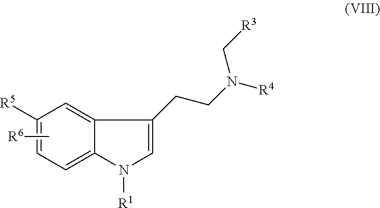

TABLE 8

| Cmpd | R¹ | R³ | R⁴ | R⁵ | R⁶ |
|---|---|---|---|---|---|
| 004 | H | 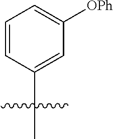 [SMILES: *c1cccc(Oc2ccccc2)c1] | H | H | H |
| 007 | H | 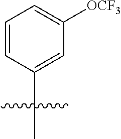 [SMILES: *c1cccc(OC(F)(F)F)c1] | H | H | H |
| 008 | H | 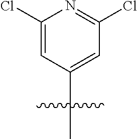 [SMILES: *c1cc(Cl)nc(Cl)c1] | H | H | H |
| 009 | H | 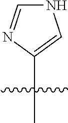 [SMILES: *c1c[nH]cn1] | H | H | H |
| 010 | H | 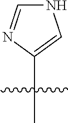 [SMILES: *c1c[nH]cn1] | 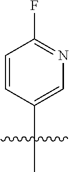 [SMILES: *c1ccc(F)nc1] | H | H |
| 011 | H | 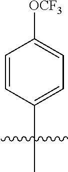 [SMILES: *c1ccc(OC(F)(F)F)cc1] | H | H | H |
| 012 | H | 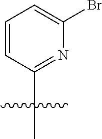 [SMILES: *c1cccc(Br)n1] | H | H | H |
| 020 | H | 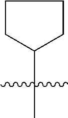 [SMILES: *C1CCCC1] | H | H | H |
| 021 | H | 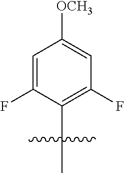 [SMILES: *c1c(F)cc(OC)cc1F] | H | H | H |
| 022 | H | 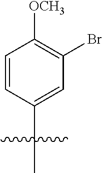 [SMILES: *c1ccc(OC)c(Br)c1] | H | H | H |
| 023 | H | 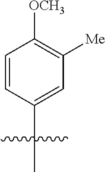 [SMILES: *c1ccc(OC)c(C)c1] | H | H | H |
| 013 | H | 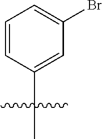 [SMILES: *c1cccc(Br)c1] | H | H | H |
| 014 | H | 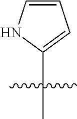 [SMILES: *c1ccc[nH]1] | H | H | H |
| 015 | H | 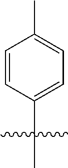 [SMILES: *c1ccc(C)cc1] | COCH₂ NHCH₂ (4-F-C₆H₄) | H | H |
| 016 | H | 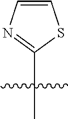 [SMILES: *c1nccs1] | H | H | H |
| 017 | H | 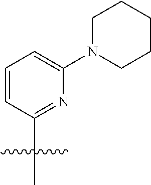 [SMILES: *c1cccc(N2CCCCC2)n1] | H | H | H |
| 017 | H | 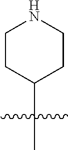 [SMILES: *C1CCNCC1] | H | H | H |
| 019 | H | 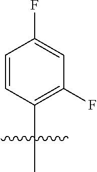 [SMILES: *c1ccc(F)cc1F] | H | H | H |
| 032 | H | 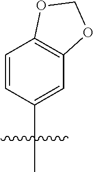 [SMILES: *c1ccc2c(c1)OCO2] | H | H | H |
| 034 | H | 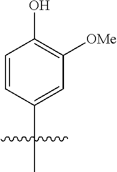 [SMILES: *c1ccc(O)c(OC)c1] | H | H | H |
| 035 | H | 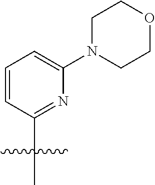 [SMILES: *c1cccc(N2CCOCC2)n1] | H | H | H |
| 036 | H | 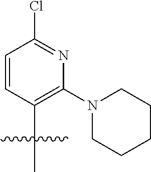 [SMILES: *c1ccc(Cl)nc1N1CCCCC1] | H | H | H |
| 024 | H | 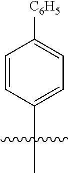 | H | H | H |
| 025 | H | 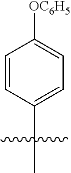 | H | H | H |
| 026 | H | 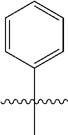 [SMILES: *c1ccccc1] | H | H | H |
| 027 | H | 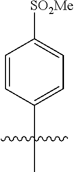 [SMILES: *c1ccc(S(C)(=O)=O)cc1] | H | H | H |
| 028 | H | 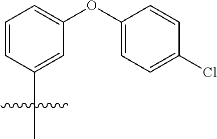 [SMILES: *c1cccc(Oc2ccc(Cl)cc2)c1] | H | H | H |
| 029 | H | 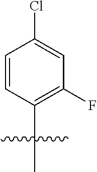 [SMILES: *c1ccc(Cl)cc1F] | H | H | H |
| 031 | H | 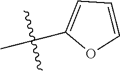 [SMILES: *c1ccco1] | H | H | H |
| 241 | 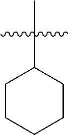 [SMILES: *C1CCCCC1] | H | 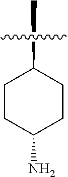 | 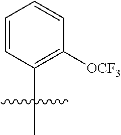 [SMILES: *c1ccccc1OC(F)(F)F] | H |
| 037 | H | 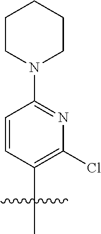 [SMILES: *c1ccc(N2CCCCC2)nc1Cl] | H | H | H |
| 081 | H | H | H | H | H |
| 082 | H | Me | H | H | H |
| | 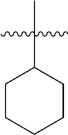 [SMILES: *C1CCCCC1] | H | 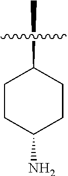 | Br | H |
| | 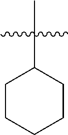 [SMILES: *C1CCCCC1] | H | 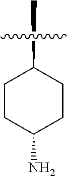 | 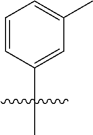 [SMILES: *c1cccc(C)c1] | H |
| 244 | 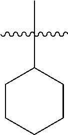 [SMILES: *C1CCCCC1] | H | 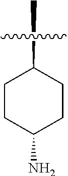 | 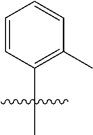 [SMILES: *c1ccccc1C] | H |
| 274 | 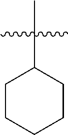 [SMILES: *C1CCCCC1] | H | 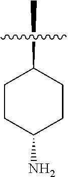 | 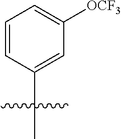 [SMILES: *c1cccc(OC(F)(F)F)c1] | H |
| 290 | 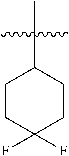 [SMILES: *C1CCC(F)(F)CC1] | H | 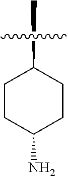 | Br | H |

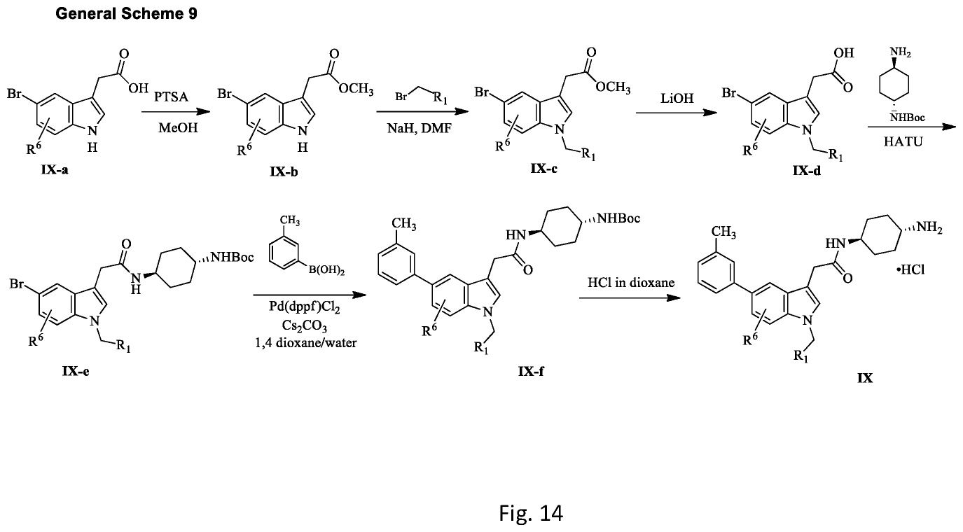

The general scheme 9 (FIG. 14) demonstrates a synthetic routed for synthesis of compound IX. Esterification of IX-a and subsequent alkylation of IX-b provided ester IX-c. Ester hydrolysis of IX-c and subsequent coupling reaction with suitable amine provides compound IX-e. Under Suzuki coupling of IX-e with boronic acid was carried out to afford compound IX-f which under acidic condition undergo deprotection and yield salt of compound IX.

### Example IX: Synthesis of N-((1R,4R)-4-aminocyclohexyl)-2-(1-(cyclohexylmethyl)-5-(m-tolyl)-1H-indol-3-yl) acetamide.hydrochloride

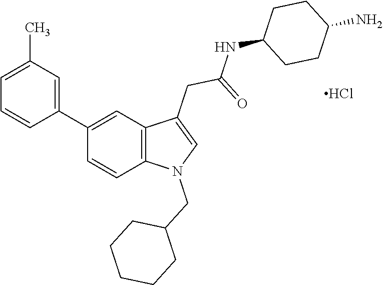 [SMILES: Cc1cccc(-c2ccc3c(c2)c(CC(=O)N[C@H]2CC[C@H](N)CC2)cn3CC2CCCCC2)c1 ; Cl]

### Synthesis of Methyl 2-(5-bromo-1H-indol-3-yl) Acetate

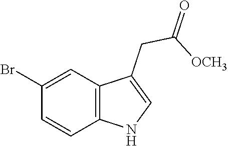 [SMILES: COC(=O)Cc1c[nH]c2ccc(Br)cc12]

A solution of 2-(5-bromo-1H-indol-3-yl) acetic acid (500 mg, 1.97 mmol) anhydrous MeOH (100 mL) was treated with PTSA (34 mg, 0.197 mmol) and heated at 75° C. for 16 h. The mixture was concentrated, the residue was dissolved in CH₂C12 (50 mL), washed with water (3×20 mL) and brine (20 mL). The CH₂C12 layer was separated, dried (Na₂SO₄), filtered and concentrated to give methyl 2-(5-bromo-1H-indol-3-yl) acetate as a dark red solid (465 mg, 88%). ESI-MS m/z 268 [M]⁺.

### Synthesis of Methyl 2-(5-bromo-1-(cyclohexylmethyl)-1H-indol-3-yl) Acetate

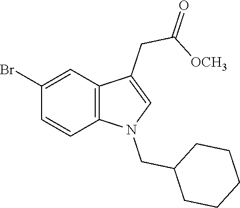 [SMILES: COC(=O)Cc1cn(CC2CCCCC2)c2ccc(Br)cc12]

To a slurry of caesium carbonate (486 mg, 1.49 mmol) in DMF (3 mL) at 0° C., a solution of methyl 2-(5-bromo-1H-indol-3-yl) acetate (200 mg, 0.746 mmol) in DMF (10 mL) was added followed by the addition of bromomethyl cyclohexane (0.156 mL, 1.12 mmol). The reaction mixture was gradually warmed to room temperature over 16 h. The reaction mixture was quenched with water, dissolved in EtOAc (50 mL), washed with water (3×20 mL) and brine (20 mL). The EtOAc layer was separated, dried (Na₂SO₄), filtered and concentrated. The residue was purified by combi-flash chromatography (silica gel, EtOAc/Hexanes) to give methyl 2-(5-bromo-1-(cyclohexylmethyl)-1H-indol-3-yl) acetate as a yellow oil (64 mg, 24%). ESI-MS m/z 364 [M]⁺.

### Synthesis of 2-(5-bromo-1-(cyclohexylmethyl)-1H-indol-3-yl) acetic Acid

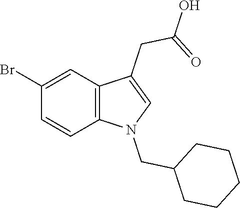 [SMILES: O=C(O)Cc1cn(CC2CCCCC2)c2ccc(Br)cc12]

2-(5-bromo-1-(cyclohexylmethyl)-1H-indol-3-yl) acetic acid was prepared by the ester hydrolysis of 180-3 (155 mg, 0.425 mmol) with lithium hydroxide (102 mg, 4.25 mmol) in MeOH/THF/H₂O (1:1:1) using the procedure described for intermediate 1-7 (Scheme 4). It was obtained as a yellow solid (126 mg, 85%). ESI-MS m/z 350 [M]⁺.

### Synthesis of Tert-Butyl ((1R,4R)-4-(2-(5-bromo-1-(cyclohexylmethyl)-1H-indol-3-yl) acetamido)cyclohexyl)carbamate

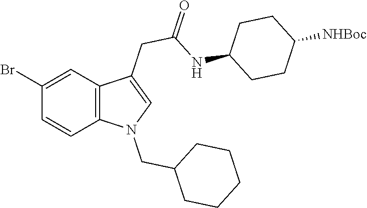 [SMILES: CC(C)(C)OC(=O)N[C@H]1CC[C@H](NC(=O)Cc2cn(CC3CCCCC3)c3ccc(Br)cc23)CC1]

tert-butyl((1r,4r)-4-(2-(5-bromo-1-(cyclohexylmethyl)-1H-indol-3-yl) acetamido) cyclo hexyl) carbamate was prepared by coupling 2-(5-bromo-1-(cyclohexylmethyl)-1H-indol-3-yl)acetic acid (86 mg, 0.245 mmol) with tert-butyl ((r,4r)-4-aminocyclohexyl) carbamate (63 mg, 0.295 mmol) with HATU (130 mg, 0.343 mmol) as the coupling reagent and DIPEA (0.08 mL, 0.49 mmol), as the base in DMF as described for the synthesis of intermediate 1-9. It was obtained as a yellow solid (74 mg, 56%). ESI-MS m/z 546 [M]⁺.

### Synthesis of Tert-Butyl ((1R,4R)-4-(2-(1-(cyclohexylmethyl)-5-(m-tolyl)-1H-indol-3-yl)acetamido)cyclohexyl)carbamate

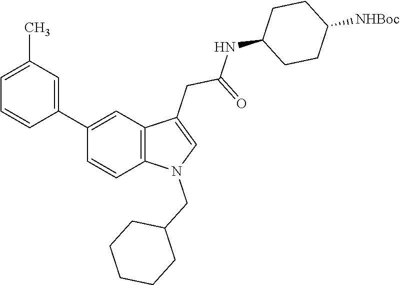 [SMILES: Cc1cccc(-c2ccc3c(c2)c(CC(=O)N[C@H]2CC[C@H](NC(=O)OC(C)(C)C)CC2)cn3CC2CCCCC2)c1]

A solution of tert-butyl ((1R,4R)-4-(2-(5-bromo-1-(cyclohexylmethyl)-1H-indol-3-yl) acetamido)cyclohexyl) carbamate (80 mg, 0.146 mmol), m-tolylboronic acid (30 mg, 0.220 mmol), caesium carbonate (142 mg, 0.438 mmol) dissolved in 1,4 dioxane (1.6 mL) and water (0.4 mL) was bubbled with Ar gas for 10 min. Pd(dppf) (5 mg, 0.007 mmol) was then added into the vial and sealed. The reaction mixture was heated at 100° C. for 16 h. It was filtered dissolved in EtOAc (20 mL), washed with water (3×10 mL) and brine (10 mL). The EtOAc layer was separated, dried (Na₂SO₄), filtered and concentrated in vacuo. The residue was dissolved in MeOH and purified by C18 reverse phase combi-flash chromatography (Acetonitrile/Water) to give tert-butyl ((1R,4R)-4-(2-(1-(cyclohexylmethyl)-5-(m-tolyl)-1H-indol-3-yl) acetamido)cyclohexyl)carbamate as a light yellow solid (16 mg, 20%). ESI-MS m/z 558 [M+H]⁺.

### Synthesis of N-((1R,4R)-4-aminocyclohexyl)-2-(1-(cyclohexylmethyl)-5-(m-tolyl)-1H-indol-3-yl)acetamide.hydrochloride

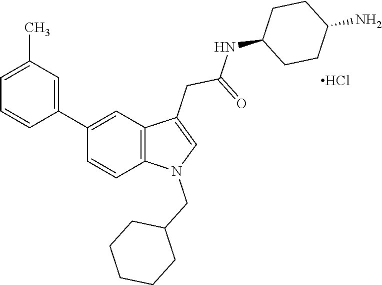 [SMILES: Cc1cccc(-c2ccc3c(c2)c(CC(=O)N[C@H]2CC[C@H](N)CC2)cn3CC2CCCCC2)c1 ; Cl]

Title compound was prepared by deprotection of the Boc group of 5 (30 mg, 0.05 mmol) with HCl in dioxane using the procedure described earlier. It was obtained as an amorphous off-white solid (6 mg, 43%). ¹H NMR (400 MHz, Methanol-d₄) δ 7.90 (d, J=7.3 Hz, 1H), 7.77 (s, 1H), 7.46-7.39 (m, 4H), 7.28 (t, J=7.6 Hz, 1H), 7.13-7.08 (m, 2H), 3.98 (d, J=7.3 Hz, 2H), 3.65 (s, 3H), 3.07-3.01 (m, 1H), 2.40 (s, 3H), 2.06-1.97 (m, 4H), 1.99-1.83 (m, 1H), 1.79-1.71 (m, 2H), 1.69-1.59 (m, 3H), 1.53-1.41 (m, 2H), 1.39-1.28 (m, 2H), 1.27-1.17 (m, 3H), 1.11-0.99 (m, 2H); HPLC (Method 5) 97.1% (AUC), t_{R}=12.62 min; ESI-MS m/z 458 [M+H]⁺.

Following the procedure described in scheme 9/Example IX, compounds of Table 9 are prepared by using suitable starting materials and proper conditions.

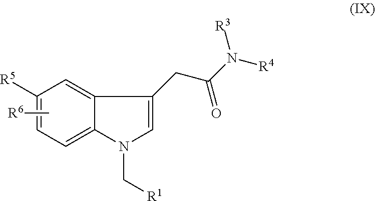

TABLE 9

| Cmpd | R¹ | R³ | R⁵ | R⁶ |
|---|---|---|---|---|
| 190 | 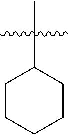 [SMILES: *C1CCCCC1] | 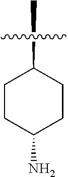 | Br | H |
| 209 | 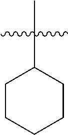 [SMILES: *C1CCCCC1] | 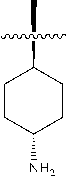 | 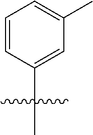 [SMILES: *c1cccc(C)c1] | H |
| 234 | 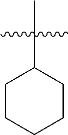 [SMILES: *C1CCCCC1] | 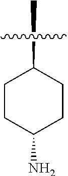 | 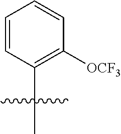 [SMILES: *c1ccccc1OC(F)(F)F] | H |
| 235 | 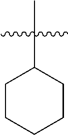 [SMILES: *C1CCCCC1] | 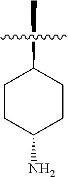 | 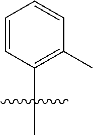 [SMILES: *c1ccccc1C] | H |
| 240 | 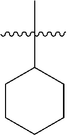 [SMILES: *C1CCCCC1] | 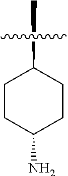 | 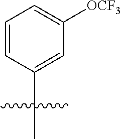 [SMILES: *c1cccc(OC(F)(F)F)c1] | H |
| 255 | 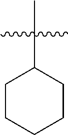 [SMILES: *C1CCCCC1] | 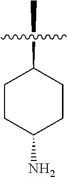 | 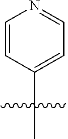 [SMILES: *c1ccncc1] | H |
| 277 | 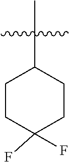 [SMILES: *C1CCC(F)(F)CC1] | 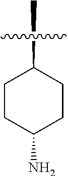 | Br | H |

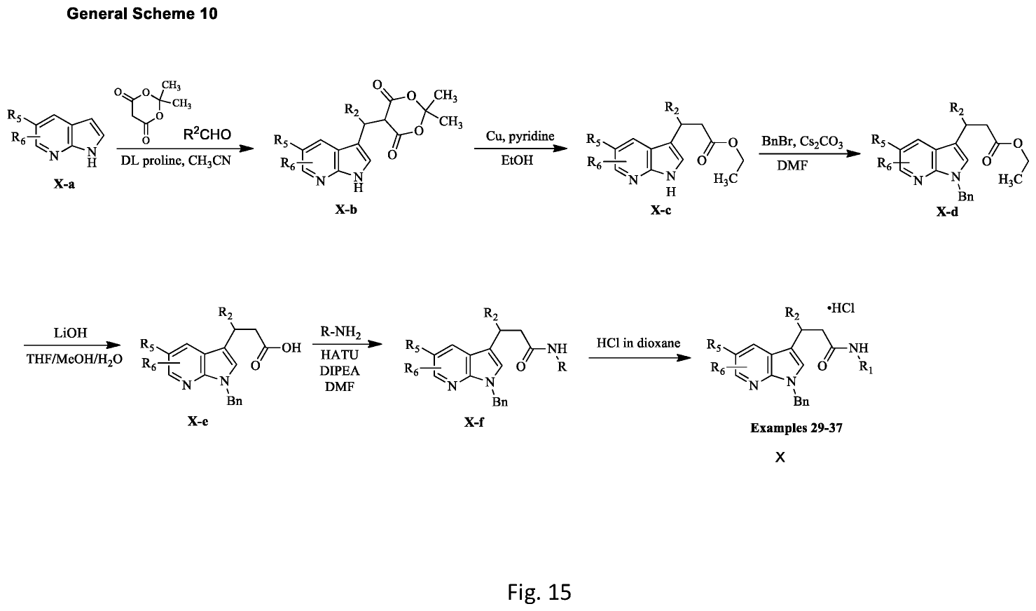

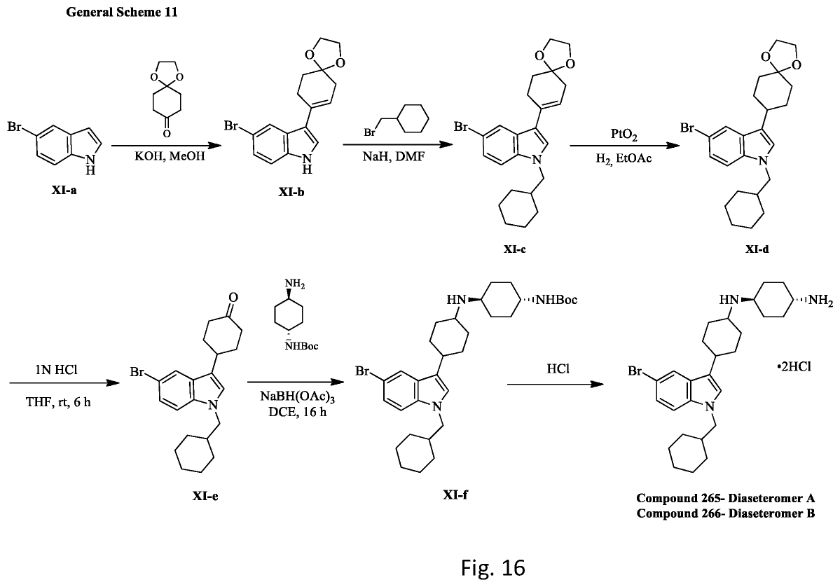

The general scheme 10 (FIG. 15) shows method of preparation of compound X. Condensation of appropriate azaindole (X-a), Meldrum's acid and aldehyde R₂CHO gave compound X-b, which under decarboxylation yielded ester derivatives X-c. N-Alkylation of X-c with benzyl halide gave compound X-d followed by hydrolysis of ester group afforded corresponding acid X-e. Treatment of X-e with appropriate NHR₃R₄ under coupling condition gave compound of formula X-f. Finally, deprotection of N-protecting group under appropriate condition provide compound X.

Compound of formula X, mentioned in Table 10, were prepared following the process of preparation of compound VA described in general scheme VA starting from appropriate azaindole/instead of indole derivatives.

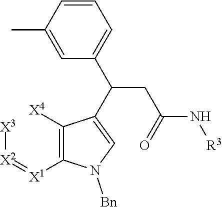

TABLE 10

| Cmpd | X | R³ |
|---|---|---|
| 030 | X¹ = N X², X³ = CH | 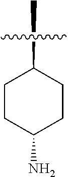 |
| 033 | X¹ = N X², X³ = CH | 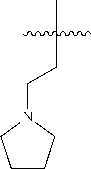 [SMILES: *CCN1CCCC1] |
| 038 | X¹ = N X², X³ = CH | 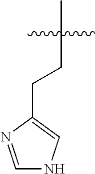 [SMILES: *CCc1c[nH]cn1] |
| 039 | X¹ = N X², X³ = CH | 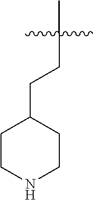 [SMILES: *CCC1CCNCC1] |
| 040 | X¹ = N X², X³ = CH | 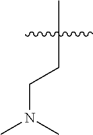 [SMILES: *CCN(C)C] |
| 041 | X¹ = N X², X³ = CH | 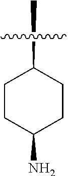 |
| 043 | X¹ = N X², X³ = CH | 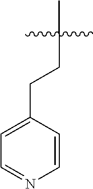 [SMILES: *CCc1ccncc1] |
| 044 | X¹ = N X², X³ = CH | 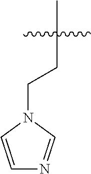 [SMILES: *CCn1ccnc1] |
| 069 | X¹ = N X², X³ = CH | 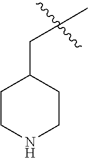 [SMILES: *CC1CCNCC1] |
| 070 | X¹ = N X², X³ = CH | 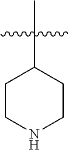 [SMILES: *C1CCNCC1] |
| 090 | X² = N X¹, X³ = CH | 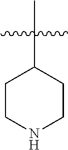 [SMILES: *C1CCNCC1] |
| 094 | X¹, X³ = N X² = CH | 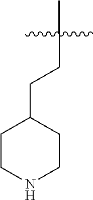 [SMILES: *CCC1CCNCC1] |
| 097 | X² = N X¹, X³ = CH | 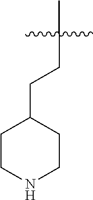 [SMILES: *CCC1CCNCC1] |
| 098 | X⁴ = N X¹, X², X³ = CH | 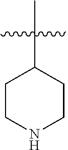 [SMILES: *C1CCNCC1] |

### Synthesis of (1R,4R)—N¹-(4-(5-bromo-1-(cyclohexylmethyl)-1H-indol-3-yl)cyclohexyl)cyclo hexane-1,4-diamine dihydrochloride (Diastereomer B -Compound 265 & 266)

See General scheme 11 (FIG. 16).

### Synthesis of 5-bromo-3-(1,4-dioxaspiro[4.5]dec-7-en-8-yl)-1H-indole (XI-b)

A mixture of 5-bromo-1H-indole (1.0 g, 5.10 mmol), 1,4-dioxaspiro[4.5]decan-8-one (795 mg, 5.10 mmol) and potassium hydroxide (16 g, 25.50 mmol) in MeOH (10 mL) was heated to reflux for 2-3 h. Reaction mixture was cooled to room temperature and water (20 mL) was added to quench the reaction. The reaction mixture was extracted with EtOAc (50 mL), washed with water (30 mL×2) and brine (15 mL). The EtOAc layer was dried (Na₂SO₄), concentrated in vacuo and the residue was purified by combi-flash chromatography (silica gel, EtOAc/Hexanes) to afford 5-bromo-3-(1,4-dioxaspiro [4.5]dec-7-en-8-yl)-1H-indole (1.50 g, 87%) as white solid. ESI MS m/z 334 [M+H]⁺.

### Synthesis of 5-bromo-1-(cyclohexylmethyl)-3-(1,4-dioxaspiro[4.5]dec-en-8-yl)-1H-indole (XI-c)

5-bromo-1-(cyclohexylmethyl)-3-(1,4-dioxaspiro[4.5]dec-7-en-8-yl)-1H-indole was prepared by N-alkylation of 5-bromo-3-(1,4-dioxaspiro[4.5]dec-7-en-8-yl)-1H-indole with (bromomethyl) cyclohexane and NaH as the base using the procedure described for the synthesis of intermediate (XI-c). It was obtained as colorless oil (70% yield). ESI MS m/z 430 [M+H]⁺.

### Synthesis of 5-bromo-1-(cyclohexylmethyl)-3-(1,4-dioxaspiro[4.5]decan-8-yl)-1H-indole(XI-d)

5-bromo-1-(cyclohexylmethyl)-3-(1,4-dioxaspiro[4.5]dec-7-en-8-yl)-1H-indole (450 mg) (5) was dissolved in 10 ml of EtOAc and to that 5 mg of platinum oxide was added. Reaction mixture was shaken at 35 PSI hydrogen gas pressure in the Parr shaker for 8 h. The reaction mixture was filtered through a celite bed and concentrated in vacuo to afford 5-bromo-1-(cyclohexylmethyl)-3-(1,4-dioxaspiro[4.5]decan-8-yl)-1H-indole (450 mg) as a semisolid, which was used as such in the next step without purification. ESI MS m/z 432 [M+H]⁺.

### Synthesis of 4-(5-bromo-1-(cyclohexylmethyl)-1H-indol-3-yl)cyclohexanone (XI-e)

5-bromo-1-(cyclohexylmethyl)-3-(1,4-dioxaspiro[4.5]decan-8-yl)-1H-indole (450 mg) was taken in the mixture of 6 ml of THE and 6 ml of N HCl. The reaction mixture was stirred at room temperature for 14 h and neutralized with a saturated solution of sodium bicarbonate. The reaction mixture was extracted with EtOAc (50 mL), washed with water (30 mL×2) and brine (15 mL). The EtOAc layer was dried (Na₂SO₄) and concentrated in vacuo to afford 4-(5-bromo-1-(cyclohexylmethyl)-1H-indol-3-yl)cyclohexanone (350 mg, 86%) semisolid mass. ESI MS m/z 388 [M+H]⁺.

### Synthesis of Tert-Butyl ((1r,4r)-4-((4-(5-bromo-1-(cyclohexylmethyl)-1H-indol-3-yl)cyclohexyl)amino)cyclohexyl)carbamate (IX-f)

tert-butyl ((1R,4R)-4-aminocyclohexyl)carbamate (145 mg, 0.68 mmol), 4-(5-bromo-1-(cyclohexylmethyl)-1H-indol-3-yl)cyclohexanone (220 mg, 0.56 mmol) and NaBH(OAc)₃ were taken in 5 mL of 1,2-dichloroethane and acetic acid (0.1 mL) was added. The reaction mixture was stirred at room temperature for 16 h and was neutralized with saturated solution of sodium bicarbonate. The reaction mixture was extracted with CH₂Cl₂ (50 ml) and washed with brine (15 mL). The CH₂Cl₂ layer was separated, dried (Na₂SO₄), concentrated in vacuo and the residue was purified by combi-flash chromatography (silica gel, EtOAc/Hexanes) to afford of tert-butyl ((1R,4R)-4-((4-(5-bromo-1-(cyclohexylmethyl)-1H-indol-3-yl)cyclohexyl) amino)cyclohexyl) carbamate (30 mg, 9%) as Diastereomer A (XI-ga), ¹H NMR (400 MHz, CDCl₃) δ 7.71 (d, J=1.7 Hz, 1H), 7.23 (dd, J=8.6, 1.8, Hz, 1H), 7.21 (d, J=1.9 Hz, 1H), 7.13 (d, J=8.7 Hz, 1H), 4.36 (bs, 1H), 3.87 (d, J=7.8 Hz, 2H), 3.39 (bs, 1H), 3.01-2.84 (m, 2H), 2.58-2.42 (m, 1H), 2.06-1.89 (m, 4H), 1.86-1.75 (m, 5H), 1.73-1.62 (m, 8H), 1.61-1.55 (m, 2H), 1.43 (s, 9H), 1.29-1.06 (m, 7H), 1.04-0.90 (m, 2H); ESI MS m/z 586 [M+H]⁺ and tert-butyl ((1R,4R)-4-((4-(5-bromo-1-(cyclohexylmethyl)-1H-indol-3-yl)cyclo hexyl) amino)cyclohexyl) carbamate (20 mg, 6%) as Diasteromer B (XI-gb) as a white solid. ¹H NMR (400 MHz, CDCl3) δ 7.71 (d, J=1.8 Hz, 1H), 7.23 (dd, J=8.6, 1.8 Hz, 1H), 7.13 (d, J=8.7 Hz, 1H), 6.77 (s, 1H), 4.36 (bs, 1H), 3.82 (d, J=7.2 Hz, 2H), 3.42 (bs, 1H), 2.78-2.59 (m, 3H), 2.14-2.05 (m, 3H), 2.04-1.96 (m, 5H), 1.94-1.87 (m, 3H), 1.81-1.74 (m, 1H), 1.72-1.64 (m, 3H), 1.61-1.55 (m, 2H), 1.53-1.48 (m, 1H), 1.43 (s, 9H), 1.34-1.30 (m, 1H), 1.26-1.18 (m, 3H), 1.17-1.09 (m, 4H), 1.01-0.89 (m, 2H); ESI MS m/z 586 [M+H]⁺.

### Synthesis of (1R,4R)—N¹-(4-(5-bromo-1-(cyclohexylmethyl)-1H-indol-3-yl)cyclohexyl)cyclo hexane-1,4-diamine Dihydrochloride (Diastereomer a -Compound 265)

(1R,4R)—N¹-(4-(5-bromo-1-(cyclohexylmethyl)-1H-indol-3-yl)cyclohexyl)cyclo hexane-1,4-diamine dihydrochloride was prepared by deprotection of the Boc group of tert-butyl ((1R,4R)-4-((4-(5-bromo-1-(cyclohexylmethyl)-1H-indol-3-yl)cyclohexyl) amino)cyclohexyl) carbamate (XI-ga), with HCl in dioxane using the procedure described elsewhere. It was obtained as an amorphous white solid (70% yield).

### Synthesis of (1R,4R)—Ni-(4-(5-bromo-1-(cyclohexylmethyl)-1H-indol-3-yl)cyclohexyl)cyclo hexane-1,4-diamine Dihydrochloride (Diastereomer B -Compound 266)

(1R,4R)—N¹-(4-(5-bromo-1-(cyclohexylmethyl)-1H-indol-3-yl)cyclohexyl)cyclo hexane-1,4-diamine dihydrochloride was prepared by deprotection of the Boc group of tert-butyl ((1R,4R)-4-((4-(5-bromo-1-(cyclohexylmethyl)-1H-indol-3-yl)cyclohexyl) amino)cyclohexyl) carbamate (XI-gb)), with HCl in dioxane using the procedure described earlier. It was obtained as an amorphous white solid (52% yield).

Salts of the compounds of formula F-I, I or any subgroup thereof can be prepared by subjecting the compound to the desired acid. The method is depicted for Compound 372 in Scheme 12.

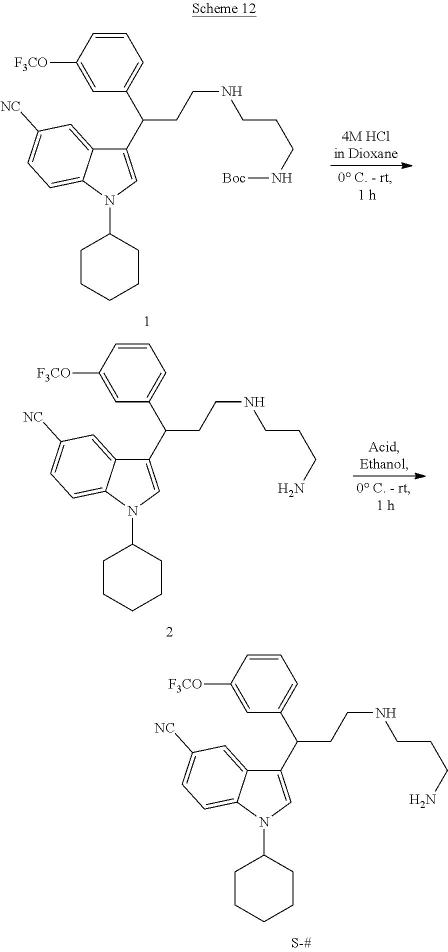

### Synthesis of 3-(3-((3-aminopropyl) amino)-1-(3-(trifluoromethoxy) phenyl) propyl)-1-cyclohexyl-1H-indole-5-carbonitrile

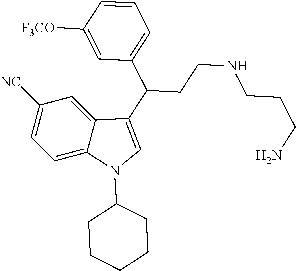 [SMILES: N#Cc1ccc2c(c1)c(C(CCNCCCN)c1cccc(OC(F)(F)F)c1)cn2C1CCCCC1]

To a stirred solution of tert-butyl (3-((3-(5-cyano-1-cyclohexyl-1H-indol-3-yl)-3-(3-(trifluoromethoxy) phenyl) propyl) amino) propyl) carbamate (500 mg, 0.836 mmol, 1 eq) in DCM (5 mL) was added 4M HCl in 1,4 Dioxane (5 mL) at 0° C. and stirred at room temperature for 1 hour. The progress of the reaction was monitored by TLC analysis. After completion of reaction, the reaction mixture was concentrated under reducer pressure to obtain crude compound. The crude compound was basified with saturated aqueous NaHCO₃ solution (20 mL), extracted with DCM (2×30 mL). The combined organic layer was dried over anhydrous sodium sulphate and concentrated under reduced pressure. The crude compound was washed with diethyl ether to afford product (yield: 350 mg, 84%) as pale yellow Solid.

¹H NMR (400 MHz, DMSO-d₆) δ 7.97 (s, 1H), 7.77 (s, 1H), 7.68 (d, J=8.6 Hz, 2H), 7.40 (d, J=9.3 Hz, 4H), 7.12 (d, J=6.7 Hz, 2H), 4.40 (s, 2H), 3.55 (s, 2H), 3.15 (s, 1H), 2.64 (s, 1H), 2.43-2.37 (m, 2H), 2.24 (s, 1H), 2.15 (s, 1H), 1.91 (s, 2H), 1.82 (s, 3H), 1.78-1.66 (m, 4H), 1.49 (d, J=12.4 Hz, 5H), 1.30 (d, J=17.8 Hz, 2H), 1.23 (d, J=10.8 Hz, 3H)

### Synthesis of 3-(3-((3-aminopropyl) amino)-1-(3-(trifluoromethoxy) phenyl) propyl)-1-cyclohexyl-1H-indole-5-carbonitrile Benzenesulfonate (S-1)

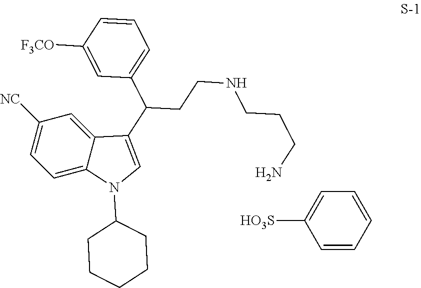 [SMILES: N#Cc1ccc2c(c1)c(C(CCNCCCN)c1cccc(OC(F)(F)F)c1)cn2C1CCCCC1 ; O=S(=O)(O)c1ccccc1]

To a stirred solution of 3-(3-((3-aminopropyl) amino)-1-(3-(trifluoromethoxy) phenyl) propyl)-1-cyclohexyl-1H-indole-5-carbonitrile (50 mg, 0.100 mmol, 1 eq) in Ethanol (2 mL) was added Benzene Sulfonic acid (19 mg, 0.12 mmol, 1.2 eq) at 0° C. and stirred the reaction mixture at room temperature for 1 h. The progress of the reaction was monitored by TLC. After completion of reaction, the reaction mixture was concentrated under reduced pressure at low temperature. The crude compound was washed with diethyl ether to afford product (yield: 38.6 mg, 58%) as white solid.

The salts of compound 372 listed in Table 11 were prepared using the appropriate acid according to method described in Scheme 12.

TABLE 11

| Salt # | Acid | Structure of Compound |
|---|---|---|
| S-1 | Benzene Sulfonic acid | 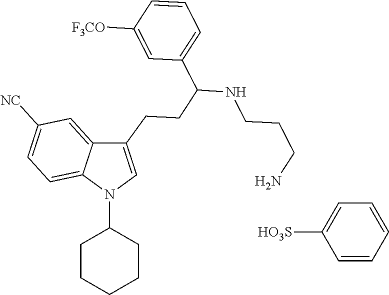 [SMILES: N#Cc1ccc2c(c1)c(CCC(NCCCN)c1cccc(OC(F)(F)F)c1)cn2C1CCCCC1 ; O=S(=O)(O)c1ccccc1] |
| S-2 | Phosphoric acid | 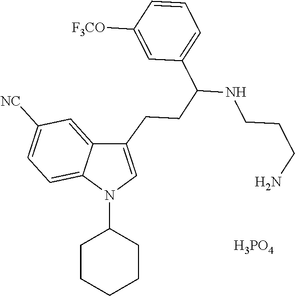 [SMILES: N#Cc1ccc2c(c1)c(CCC(NCCCN)c1cccc(OC(F)(F)F)c1)cn2C1CCCCC1 ; "H3PO4"] |
| S-3 | Tartaric acid | 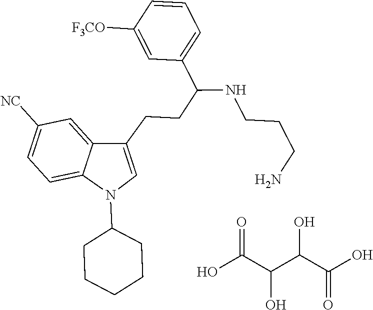 [SMILES: N#Cc1ccc2c(c1)c(CCC(NCCCN)c1cccc(OC(F)(F)F)c1)cn2C1CCCCC1 ; O=C(O)C(O)C(O)C(=O)O] |
| S-4 | Citric acid | 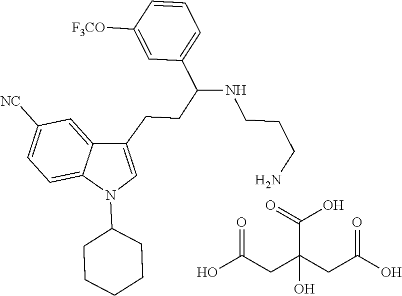 |
| S-5 | Methanesulfonic acid | 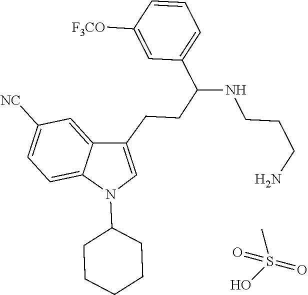 |
| S-6 | Benzoic acid | 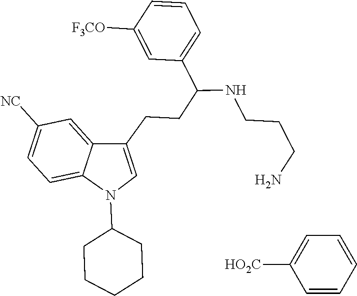 [SMILES: N#Cc1ccc2c(c1)c(CCC(NCCCN)c1cccc(OC(F)(F)F)c1)cn2C1CCCCC1 ; O=C(O)c1ccccc1] |
| S-7 | Maleic acid | 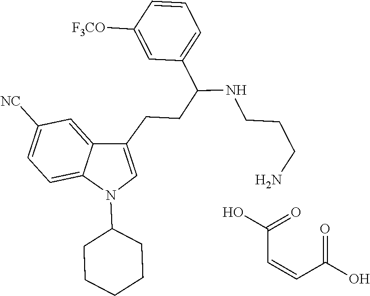 |

Characterisation of the Synthesised Compounds

Table XI below provides LC-MS data on the compounds synthesised and indicates which general synthetic method (Scheme number) was used to obtain the compound.

| | | | | Exact | | |
|---|---|---|---|---|---|---|
| | | | Exact | Mass | | |
| | | | Mass | Free | LCMS | Scheme |
| Cmpd | Structure | Name | (Salt) | base | (m/z) | no |
| 2 | 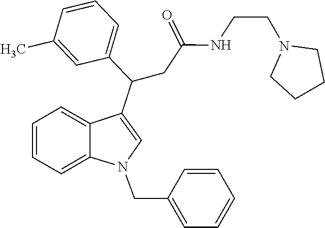 [SMILES: Cc1cccc(C(CC(=O)NCCN2CCCC2)c2cn(Cc3ccccc3)c3ccccc23)c1] | 3-(1-benzyl- 1H-indol-3-yl)- N-(2-(pyrrolidin- 1-yl)ethyl)- 3-(m-tolyl)propanamide | 465.64 | 465.65 | NA | V |
| 3 | 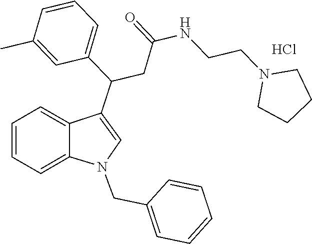 [SMILES: Cc1cccc(C(CC(=O)NCCN2CCCC2)c2cn(Cc3ccccc3)c3ccccc23)c1 ; Cl] | 3-(1-benzyl-1H- indol-3-yl)- N-(2-(pyrrolidin- 1-yl)ethyl)- 3-(m-tolyl)propanamide hydrochloride salt | 502.09 | 468.28 | NA | V |
| 7 | 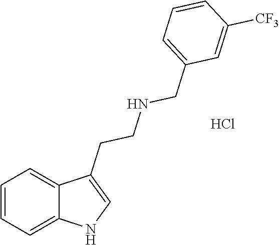 [SMILES: FC(F)(F)c1cccc(CNCCc2c[nH]c3ccccc23)c1 ; Cl] | 2-(1H-indol-3-yl)-N-(3- (trifluoromethyl) benzyl)ethan- 1-amine hydrochloride salt | 354.8 | 318.13 | 319 | VIII |
| 8 | 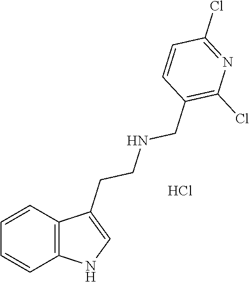 [SMILES: Clc1ccc(CNCCc2c[nH]c3ccccc23)c(Cl)n1 ; Cl] | N-((2,6-dichloropyridin-3- yl)methyl)-2-(1H-indol-3- yl)ethan-1-amine hydrochloride salt | 356.68 | 319.06 | 320 | VIII |
| 9 | 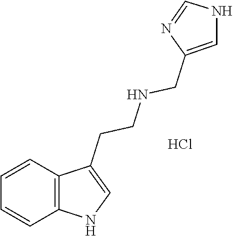 [SMILES: c1ccc2c(CCNCc3c[nH]cn3)c[nH]c2c1 ; Cl] | N-((1H-imidazol-4- yl)methyl)-2-(1H-indol-3- yl)ethan-1-amine hydrochloride salt | 276.76 | 240.14 | 241 | VIII |
| 10 | 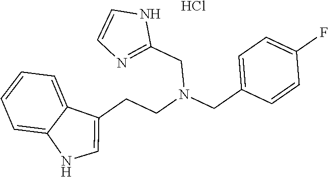 [SMILES: Cl ; Fc1ccc(CN(CCc2c[nH]c3ccccc23)Cc2ncc[nH]2)cc1] | N-((1H-imidazol-2- yl)methyl)-N-(4- fluorobenzyl)-2-(1H-indol- 3-yl)ethan-1-amine hydrochloride salt | 384.88 | 348.18 | 349 | VIII |
| 11 | 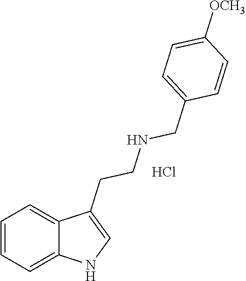 [SMILES: COc1ccc(CNCCc2c[nH]c3ccccc23)cc1 ; Cl] | 2-(1H-indol-3-yl)-N-(4- methoxybenzyl)ethan-1- amine hydrochloride salt | 316.83 | 280.16 | 281 | VIII |
| 12 | 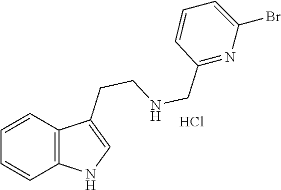 [SMILES: Brc1cccc(CNCCc2c[nH]c3ccccc23)n1 ; Cl] | N-((6-bromopyridin-2- yl)methyl)-2-(1H-indol-3- yl)ethan-1-amine hydrochloride salt | 366.68 | 329.05 | | VIII |
| 13 | 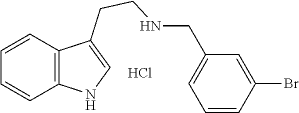 [SMILES: Brc1cccc(CNCCc2c[nH]c3ccccc23)c1 ; Cl] | N-(3-bromobenzyl)-2-(1H- indol-3-yl)ethan-1-amine hydrochloride salt | 365.7 | 328.06 | 329 | VIII |
| 14 | 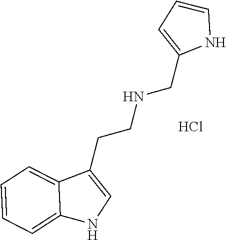 [SMILES: c1c[nH]c(CNCCc2c[nH]c3ccccc23)c1 ; Cl] | N-((1H-pyrrol-2- yl)methyl)- 2-(1H-indol-3-yl)ethan-1- amine hydrochloride salt | 275.78 | 239.14 | 240 | VIII |
| 15 | 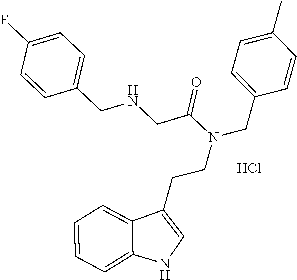 [SMILES: Cc1ccc(CN(CCc2c[nH]c3ccccc23)C(=O)CNCc2ccc(F)cc2)cc1 ; Cl] | N-(2-(1H-indol-3-yl)ethyl)- 2-((4-fluorobenzyl)amino)- N-(4- methylbenzyl)acetamide hydrochloride salt | 465.99 | 429.22 | 430 | VIII |
| 16 | 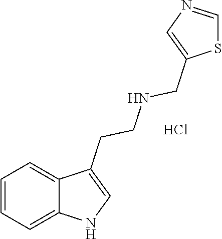 [SMILES: c1ccc2c(CCNCc3cncs3)c[nH]c2c1 ; Cl] | 2-(1H-indol-3-yl)-N- (thiazol-5-ylmethyl) ethan-1- amine hydrochloride salt | 293.81 | 257.1 | 258 | VIII |
| 17 | 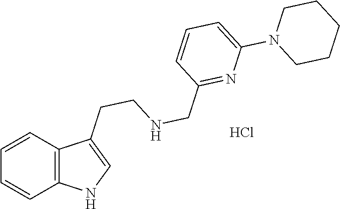 [SMILES: c1cc(CNCCc2c[nH]c3ccccc23)nc(N2CCCCC2)c1 ; Cl] | 2-(1H-indol-3-yl)-N-((6- (piperidin-1-yl)pyridin-2- yl)methyl)ethan-1-amine hydrochloride salt | 370.92 | 334.22 | 335 | VIII |
| 18 | 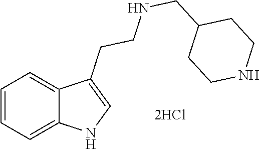 [SMILES: c1ccc2c(CCNCC3CCNCC3)c[nH]c2c1 ; "2HCl"] | 2-(1H-indol-3-yl)-N- (piperidin-4- ylmethyl)ethan- 1-amine hydrochloride salt | 330.3 | 257.19 | 258 | VIII |
| 19 | 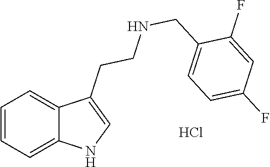 [SMILES: Fc1ccc(CNCCc2c[nH]c3ccccc23)c(F)c1 ; Cl] | N-(2,4-difluorobenzyl)-2- (1H-indol-3-yl)ethan-1- amine hydrochloride salt | 322.78 | 286.13 | 287 | VIII |
| 20 | 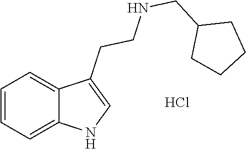 [SMILES: c1ccc2c(CCNCC3CCCC3)c[nH]c2c1 ; Cl] | N-(cyclopentylmethyl)-2- (1H-indol-3-yl)ethan-1- amine hydrochloride salt | 278.82 | 242.18 | 243 | VIII |
| 21 | 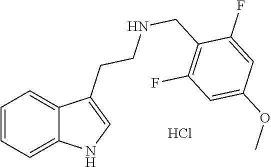 [SMILES: COc1cc(F)c(CNCCc2c[nH]c3ccccc23)c(F)c1 ; Cl] | N-(2,6-difluoro-4- methoxybenzyl)-2-(1H- indol-3-yl)ethan-1-amine hydrochloride salt | 352.81 | 316.14 | 317 | VIII |
| 22 | 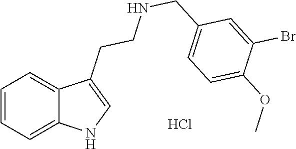 [SMILES: COc1ccc(CNCCc2c[nH]c3ccccc23)cc1Br ; Cl] | N-(3-bromo-4- methoxybenzyl)-2-(1H- indol-3-yl)ethan-1-amine hydrochloride salt | 395.72 | 358.07 | 359 | VIII |
| 23 | 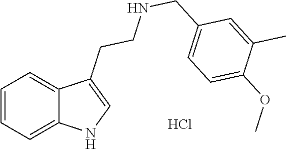 [SMILES: COc1ccc(CNCCc2c[nH]c3ccccc23)cc1C ; Cl] | 2-(1H-indol-3-yl)-N-(4- methoxy-3- methylbenzyl) ethan-1-amine hydrochloride salt | 330.85 | 294.17 | 295 | VIII |
| 24 | 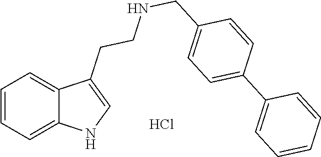 [SMILES: c1ccc(-c2ccc(CNCCc3c[nH]c4ccccc34)cc2)cc1 ; Cl] | N-([1,1′-biphenyl]-4- ylmethyl)-2-(1H-indol-3- yl)ethan-1-amine hydrochloride salt | 362.9 | 326.18 | 327 | VIII |
| 25 | 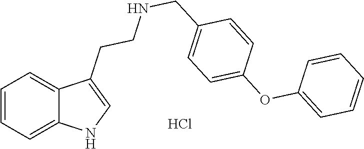 [SMILES: c1ccc(Oc2ccc(CNCCc3c[nH]c4ccccc34)cc2)cc1 ; Cl] | 2-(1H-indol-3-yl)-N-(4- phenoxybenzyl)ethan-1- amine hydrochloride salt | 378.89 | 342.17 | 343 | VIII |
| 26 | 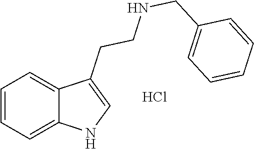 [SMILES: c1ccc(CNCCc2c[nH]c3ccccc23)cc1 ; Cl] | N-benzyl-2-(1H-indol-3- yl)ethan-1-amine hydrochloride salt | 286.8 | 250.15 | 251 | VIII |
| 27 | 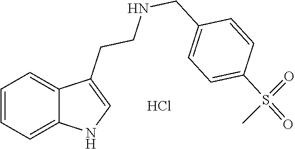 [SMILES: CS(=O)(=O)c1ccc(CNCCc2c[nH]c3ccccc23)cc1 ; Cl] | 2-(1H-indol-3-yl)-N-(4- (methylsulfonyl) benzyl)ethan- 1-amine hydrochloride | 364.89 | 328.12 | 329 | VIII |
| 28 | 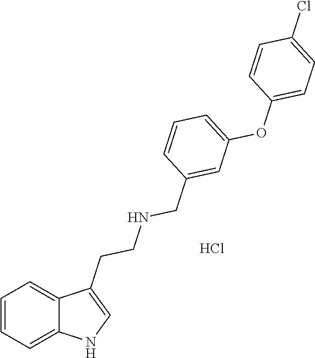 [SMILES: Clc1ccc(Oc2cccc(CNCCc3c[nH]c4ccccc34)c2)cc1 ; Cl] | N-(3-(4- chlorophenoxy)benzyl)-2- (1H-indol-3-yl)ethan-1- amine hydrochloride salt | 413.34 | 376.13 | 377 | VIII |
| 29 | 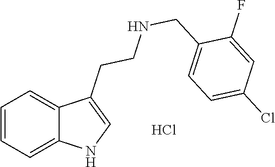 [SMILES: Fc1cc(Cl)ccc1CNCCc1c[nH]c2ccccc12 ; Cl] | N-(4-chloro-2- fluorobenzyl)- 2-(1H-indol- 3-yl)ethan-1-amine hydrochloride salt | 339.23 | 302.1 | 303 | VIII |
| 30 | 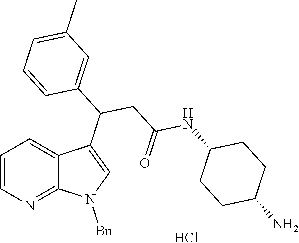 [SMILES: Cc1cccc(C(CC(=O)N[C@H]2CC[C@@H](N)CC2)c2cn(Cc3ccccc3)c3ncccc23)c1 ; Cl] | N-((1s,4s)-4- aminocyclohexyl)-3-(1- benzyl-1H-pyrrolo[2,3- b]pyridin-3-yl)-3-(m- tolyl)propanamide hydrochloride salt | 502.25 | 466.27 | NA | X |
| 31 | 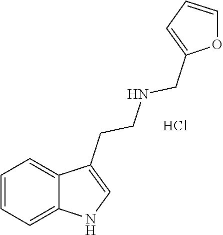 [SMILES: c1coc(CNCCc2c[nH]c3ccccc23)c1 ; Cl] | N-(furan-2-ylmethyl)-2- (1H-indol-3-yl)ethan-1- amine hydrochloride salt | 276.76 | 240.13 | NA | VIII |
| 32 | 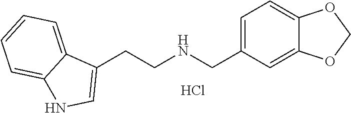 [SMILES: c1ccc2c(CCNCc3ccc4c(c3)OCO4)c[nH]c2c1 ; Cl] | N-(benzo[d][1,3]dioxol-5- ylmethyl)-2-(1H-indol-3- yl)ethan-1-amine hydrochloride salt | 330.81 | 294.14 | 295 | VIII |
| 33 | 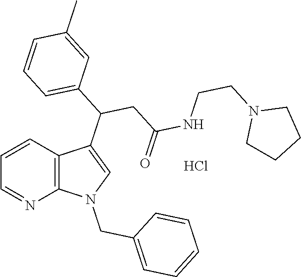 [SMILES: Cc1cccc(C(CC(=O)NCCN2CCCC2)c2cn(Cc3ccccc3)c3ncccc23)c1 ; Cl] | 3-(1-benzyl- 1H-pyrrolo[2,3- b]pyridin-3-yl)-N-(2- (pyrrolidin-1- yl)ethyl)-3-(m- tolyl)propanamide hydrochloride salt | 503.08 | 466.26 | NA | X |
| 34 | 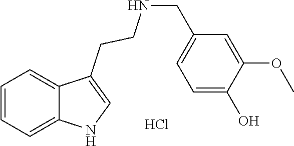 [SMILES: COc1cc(CNCCc2c[nH]c3ccccc23)ccc1O ; Cl] | 4-((2-(1H-indol-3- yl)ethyl)amino)methyl)-2- methoxyphenol hydrochloride salt | 332.82 | 296.15 | 297 | VIII |
| 35 | 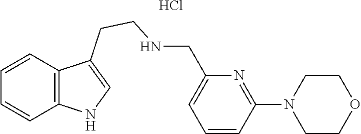 [SMILES: Cl ; c1cc(CNCCc2c[nH]c3ccccc23)nc(N2CCOCC2)c1] | 2-(1H-indol-3-yl)-N-((6- morpholinopyridin-2- yl)methyl)ethan-1-amine hydrochloride salt | 372.89 | 336.20 | 337 | VIII |
| 36 | 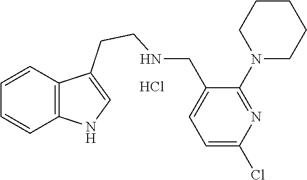 [SMILES: Clc1ccc(CNCCc2c[nH]c3ccccc23)c(N2CCCCC2)n1 ; Cl] | N-((6-chloro- 2-(piperidin-1- yl)pyridin-3- yl)methyl)-2- (1H-indol-3- yl)ethan-1- amine hydrochloride salt | 405.36 | 368.18 | 369 | VIII |
| 37 | 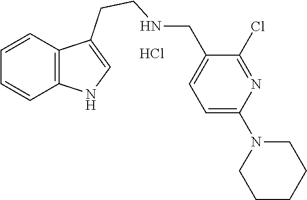 [SMILES: Clc1nc(N2CCCCC2)ccc1CNCCc1c[nH]c2ccccc12 ; Cl] | N-((2-chloro-6- (piperidin-1- yl)pyridin-3-yl) methyl)-2- (1H-indol-3-yl)ethan-1- amine hydrochloride salt | 405.36 | 368.18 | NA | VIII |
| 38 | 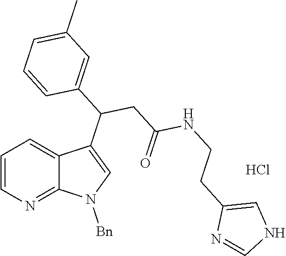 [SMILES: Cc1cccc(C(CC(=O)NCCc2c[nH]cn2)c2cn(Cc3ccccc3)c3ncccc23)c1 ; Cl] | N-(2-(1H-imidazol-4- yl)ethyl)-3-(1-benzyl-1H- pyrrolo[2,3-b] pyridin-3-yl)- 3-(m-tolyl)propanamide hydrochloride salt | 500.03 | 463.24 | 464 | X |
| 39 | 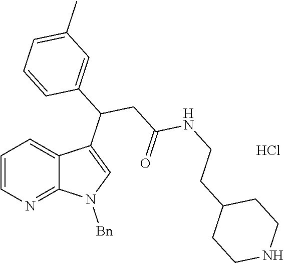 [SMILES: Cc1cccc(C(CC(=O)NCCC2CCNCC2)c2cn(Cc3ccccc3)c3ncccc23)c1 ; Cl] | 3-(1-benzyl-1H- pyrrolo[2,3- b]pyridin-3-yl)-N-(2- (piperidin-4-yl) ethyl)-3-(m- tolyl)propanamide hydrochloride salt | 517.1 | 480.29 | 481 | X |
| 40 | 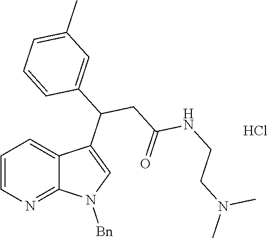 [SMILES: Cc1cccc(C(CC(=O)NCCN(C)C)c2cn(Cc3ccccc3)c3ncccc23)c1 ; Cl] | 3-(1-benzyl- 1H-pyrrolo[2,3- b]pyridin-3-yl)-N-(2- (dimethylamino)ethyl)-3- (m-tolyl)propanamide hydrochloride salt | 477.04 | 440.26 | 441 | X |
| 41 | 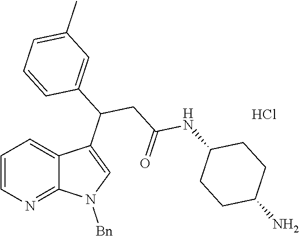 [SMILES: Cc1cccc(C(CC(=O)N[C@H]2CC[C@@H](N)CC2)c2cn(Cc3ccccc3)c3ncccc23)c1 ; Cl] | N-((1s,4s)-4- aminocyclohexyl)-3-(1- benzyl-1H-pyrrolo[2,3- b]pyridin-3-yl)-3-(m- tolyl)propanamide hydrochloride salt | 503.08 | 466.27 | 467 | X |
| 43 | 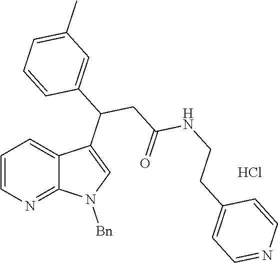 [SMILES: Cc1cccc(C(CC(=O)NCCc2ccncc2)c2cn(Cc3ccccc3)c3ncccc23)c1 ; Cl] | 3-(1-benzyl-1H- pyrrolo[2,3- b]pyridin-3-yl)-N-(2- (pyridin-4-yl)ethyl)-3-(m- tolyl)propanamide hydrochloride salt | 511.06 | 474.24 | 475 | X |
| 44 | 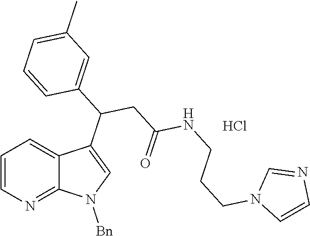 [SMILES: Cc1cccc(C(CC(=O)NCCCn2ccnc2)c2cn(Cc3ccccc3)c3ncccc23)c1 ; Cl] | N-(3-(1H-imidazol-1- yl)propyl)-3-(1-benzyl-1H- pyrrolo[2,3-b]pyridin-3-yl)- 3-(m-tolyl)propanamide | 514.06 | 477.25 | 478 | X |
| 45 | 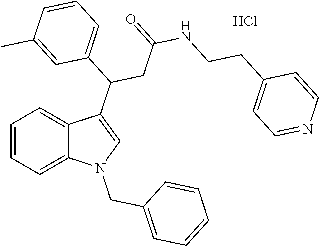 [SMILES: Cc1cccc(C(CC(=O)NCCc2ccncc2)c2cn(Cc3ccccc3)c3ccccc23)c1 ; Cl] | 3-(1-benzyl-1H-indol-3-yl)- N-(2-(pyridin-4-yl)ethyl)-3- (m-tolyl)propanamide hydrochloride salt | 510.07 | 473.25 | 474 | V |
| 46 | 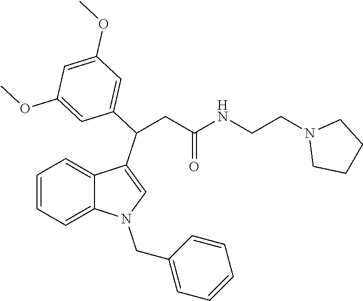 [SMILES: COc1cc(OC)cc(C(CC(=O)NCCN2CCCC2)c2cn(Cc3ccccc3)c3ccccc23)c1] | 3-(1-benzyl-1H-indol-3-yl)- 3-(3,5-dimethoxyphenyl)-N- (2-(pyrrolidin-1- yl)ethyl)propanamide hydrochloride salt | NA | 511.65 | NA | V |
| 47 | 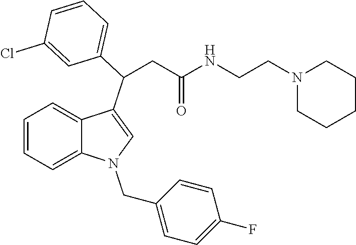 [SMILES: O=C(CC(c1cccc(Cl)c1)c1cn(Cc2ccc(F)cc2)c2ccccc12)NCCN1CCCCC1] | 3-(3-chlorophenyl)-3-(1-(4- fluorobenzyl)-1H-indol-3- yl)-N-(2-(piperidin-1- yl)ethyl)propanamide hydrochloride salt | NA | 517.06 | NA | V |
| 49 | 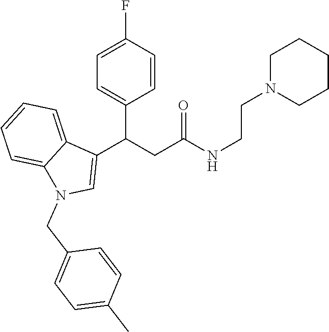 [SMILES: Cc1ccc(Cn2cc(C(CC(=O)NCCN3CCCCC3)c3ccc(F)cc3)c3ccccc32)cc1] | 3-(4-fluorophenyl)-3-(1-(4- methylbenzyl)-1H-indol-3- yl)-N-(2-(piperidin-1- yl)ethyl)propanamide hydrochloride salt | NA | 497.65 | NA | V |
| 50 | 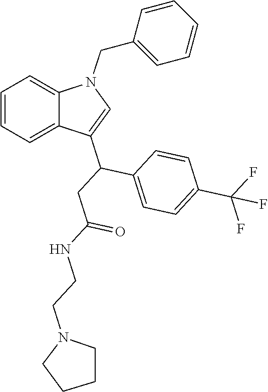 [SMILES: O=C(CC(c1ccc(C(F)(F)F)cc1)c1cn(Cc2ccccc2)c2ccccc12)NCCN1CCCC1] | 3-(1-benzyl-1H- indol-3-yl)- N-(2-(pyrrolidin- 1-yl)ethyl)-3-(4- (trifluoromethyl)phenyl) propanamide hydrochloride salt | NA | 519.6 | NA | V |
| 51 | 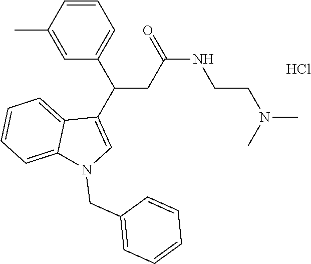 [SMILES: Cc1cccc(C(CC(=O)NCCN(C)C)c2cn(Cc3ccccc3)c3ccccc23)c1 ; Cl] | 3-(1-benzyl-1H- indol-3-yl)-N-(2- (dimethylamino)ethyl)-3- (m-tolyl)propanamide hydrochloride salt | 476.05 | 439.26 | 440 | V |
| 52 | 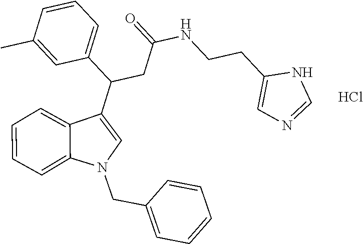 [SMILES: Cc1cccc(C(CC(=O)NCCc2cnc[nH]2)c2cn(Cc3ccccc3)c3ccccc23)c1 ; Cl] | N-(2-(1H-imidazol-5- yl)ethyl)-3-(1-benzyl-1H- indol-3-yl)-3-(m- tolyl)propanamide hydrochloride salt | 499.05 | 462.24 | 463 | V |
| 53 | 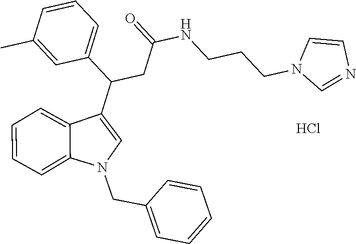 [SMILES: Cc1cccc(C(CC(=O)NCCCn2ccnc2)c2cn(Cc3ccccc3)c3ccccc23)c1 ; Cl] | N-(3-(1H-imidazol-1- yl)propyl)-3-(1- benzyl-1H- indol-3-yl)-3-(m- tolyl)propanamide hydrochloride salt | 513.07 | 476.26 | 463 | V |
| 54 | 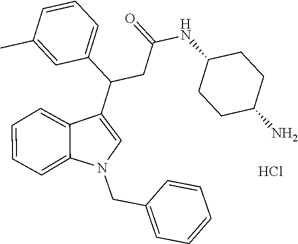 [SMILES: Cc1cccc(C(CC(=O)N[C@H]2CC[C@@H](N)CC2)c2cn(Cc3ccccc3)c3ccccc23)c1 ; Cl] | N-((1S,4S)-4- aminocyclohexyl)-3-(1- benzyl-1H-indol- 3-yl)-3-(m- tolyl)propanamide hydrochloride salt | 502.09 | 465.28 | 466 | V |
| 55 | 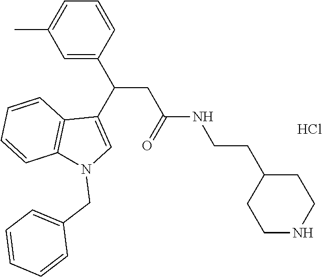 [SMILES: Cc1cccc(C(CC(=O)NCCC2CCNCC2)c2cn(Cc3ccccc3)c3ccccc23)c1 ; Cl] | 3-(1-benzyl-1H- indol-3-yl)- N-(2-(piperidin- 4-yl)ethyl)- 3-(m-tolyl)propanamide hydrochloride salt | 516.12 | 479.29 | 480 | V |
| 58 | 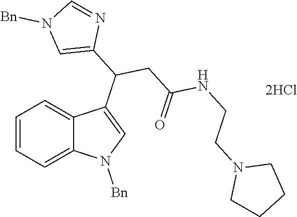 [SMILES: O=C(CC(c1cn(Cc2ccccc2)cn1)c1cn(Cc2ccccc2)c2ccccc12)NCCN1CCCC1 ; "2HCl"] | 3-(1-benzyl-1H- imidazol-4- yl)-3-(1-benzyl- 1H-indol-3- yl)-N-(2-(pyrrolidin-1- yl)ethyl)propanamide hydrochloride salt | 604.61 | 531.20 | NA | V |
| 59 | 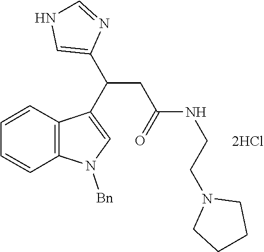 [SMILES: O=C(CC(c1c[nH]cn1)c1cn(Cc2ccccc2)c2ccccc12)NCCN1CCCC1 ; "2HCl"] | 3-(1-benzyl-1H- indol-3-yl)- 3-(1H-imidazol- 4-yl)-N-(2- (pyrrolidin-1- yl)ethyl)propanamide hydrochloride salt | 514.49 | 441.25 | 442 | V |
| 60 | 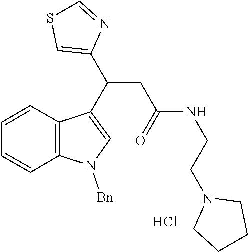 [SMILES: O=C(CC(c1cscn1)c1cn(Cc2ccccc2)c2ccccc12)NCCN1CCCC1 ; Cl] | 3-(1-benzyl-1H- indol-3-yl)- N-(2-(pyrrolidin- 1-yl)ethyl)- 3-(thiazol-4-yl) propanamide hydrochloride salt | 495.08 | 458.21 | 459 | V |
| 61 | 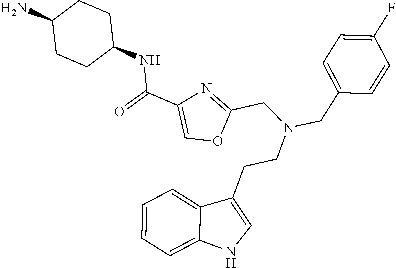 [SMILES: N[C@H]1CC[C@@H](NC(=O)c2coc(CN(CCc3c[nH]c4ccccc34)Cc3ccc(F)cc3)n2)CC1] | 2-(((2-(1H-indol-3- yl)ethyl)(4- fluorobenzyl) amino)methyl)- N-((1s,4s)-4- aminocyclohexyl) oxazole-4- carboxamide hydrochloride salt | 489.58 | 489.58 | NA | VII |
| 62 | 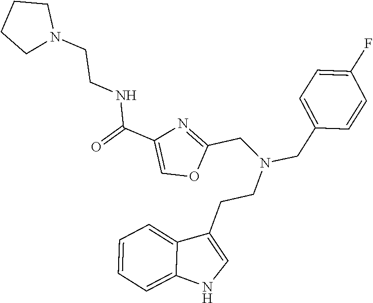 [SMILES: O=C(NCCN1CCCC1)c1coc(CN(CCc2c[nH]c3ccccc23)Cc2ccc(F)cc2)n1] | 2-(((2-(1H-indol-3- yl)ethyl)(4- fluorobenzyl) amino)methyl)- N-(2-(pyrrolidin-1- yl)ethyl)oxazole-4- carboxamide hydrochloride salt | NA | 489.58 | 490 | VII |
| 63 | 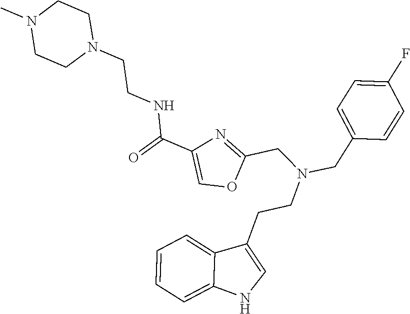 [SMILES: CN1CCN(CCNC(=O)c2coc(CN(CCc3c[nH]c4ccccc34)Cc3ccc(F)cc3)n2)CC1] | 2-(((2-(1H-indol-3- yl)ethyl)(4- fluorobenzyl) amino)methyl)- N-(2-(4- methylpiperazin-1- yl)ethyl)oxazole-4- carboxamide hydrochloride salt | | 518.63 | 519 | VII |
| 64 | 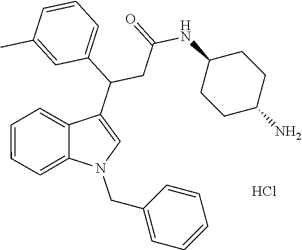 [SMILES: Cc1cccc(C(CC(=O)N[C@H]2CC[C@H](N)CC2)c2cn(Cc3ccccc3)c3ccccc23)c1 ; Cl] | N-((1R,4R)-4- aminocyclohexyl)-3-(1- benzyl-1H-indol- 3-yl)-3-(m- tolyl)propanamide salt | 502.09 | 465.28 | 466 | V |
| 66 | 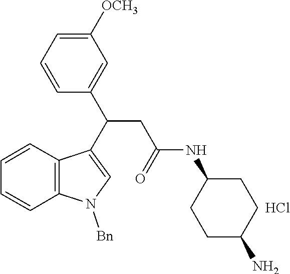 [SMILES: COc1cccc(C(CC(=O)N[C@H]2CC[C@@H](N)CC2)c2cn(Cc3ccccc3)c3ccccc23)c1 ; Cl] | N-((1S,4S)-4- aminocyclohexyl)-3-(1- benzyl-1H-indol- 3-yl)-3-(3- methoxyphenyl) propanamide hydrochloride salt | 518.09 | 481.27 | 482 | V |
| 67 | 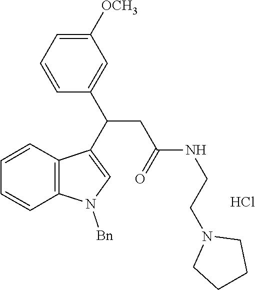 [SMILES: COc1cccc(C(CC(=O)NCCN2CCCC2)c2cn(Cc3ccccc3)c3ccccc23)c1 ; Cl] | 3-(1-benzyl- 1H-indol-3-yl)- 3-(3-methoxyphenyl)- N-(2-(pyrrolidin-1- yl)ethyl)propanamide hydrochloride salt | 518.09 | | 482 | V |
| 68 | 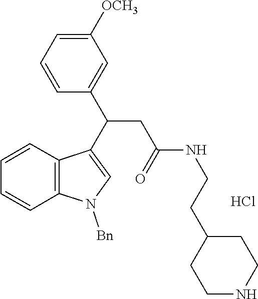 [SMILES: COc1cccc(C(CC(=O)NCCC2CCNCC2)c2cn(Cc3ccccc3)c3ccccc23)c1 ; Cl] | 3-(1-benzyl-1H- indol-3-yl)- 3-(3-methoxyphenyl)- N-(2-(piperidin-4- yl)ethyl)propanamide hydrochloride salt | 532.12 | 495.29 | 496 | V |
| 69 | 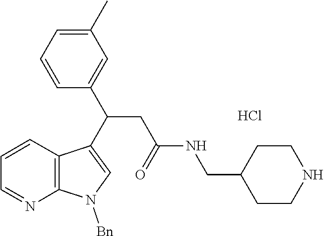 [SMILES: Cc1cccc(C(CC(=O)NCC2CCNCC2)c2cn(Cc3ccccc3)c3ncccc23)c1 ; Cl] | 3-(1-benzyl- 1H-pyrrolo[2,3- b]pyridin-3-yl)-N- (piperidin-4-ylmethyl)-3- (m-tolyl)propanamide hydrochloride salt | 503.08 | 466.27 | 467 | X |
| 70 | 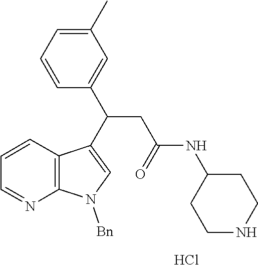 [SMILES: Cc1cccc(C(CC(=O)NC2CCNCC2)c2cn(Cc3ccccc3)c3ncccc23)c1 ; Cl] | 3-(1-benzyl-1H- pyrrolo[2,3- b]pyridin-3-yl)-N- (piperidin-4-yl)-3-(m- tolyl)propanamide hydrochloride salt | 489.05 | 452.26 | 453 | X |
| 71 | 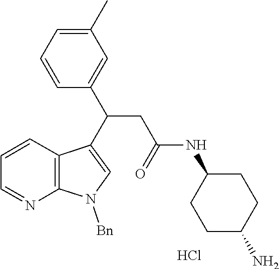 [SMILES: Cc1cccc(C(CC(=O)N[C@H]2CC[C@H](N)CC2)c2cn(Cc3ccccc3)c3ncccc23)c1 ; Cl] | N-((1R,4R)-4- aminocyclohexyl)-3-(1- benzyl-1H-pyrrolo[2,3- b]pyridin-3-yl)-3-(m- tolyl)propanamide hydrochloride salt | 503.08 | 466.27 | NA | X |
| 72 | 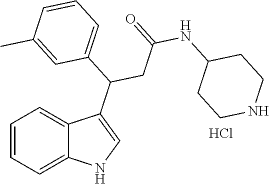 [SMILES: Cc1cccc(C(CC(=O)NC2CCNCC2)c2c[nH]c3ccccc23)c1 ; Cl] | 3-(1H-indol-3-yl)-N- (piperidin-4-yl)-3-(m- tolyl)propanamide hydrochloride salt | 397.94 | 361.22 | NA | V |
| 73 | 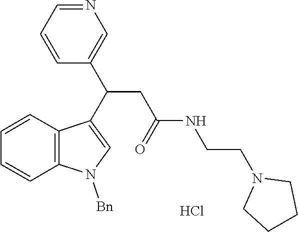 [SMILES: O=C(CC(c1cccnc1)c1cn(Cc2ccccc2)c2ccccc12)NCCN1CCCC1 ; Cl] | 3-(1-benzyl- 1H-indol-3-yl)- 3-(pyridin-3-yl)-N-(2- (pyrrolidin-1- yl)ethyl)propanamide hydrochloride salt | 489.05 | 452.26 | 453 | V |
| 74 | 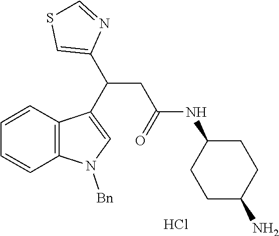 [SMILES: N[C@H]1CC[C@@H](NC(=O)CC(c2cscn2)c2cn(Cc3ccccc3)c3ccccc23)CC1 ; Cl] | N-((1s,4s)-4- aminocyclohexyl)-3-(1- benzyl-1H-indol-3-yl)-3- (thiazol-4-yl)propanamide hydrochloride salt | 495.08 | 458.21 | 459 | V |
| 75 | 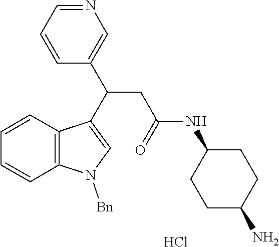 [SMILES: N[C@H]1CC[C@@H](NC(=O)CC(c2cccnc2)c2cn(Cc3ccccc3)c3ccccc23)CC1 ; Cl] | N-((1s,4s)-4- aminocyclohexyl)-3-(1- benzyl-1H-indol-3-yl)-3- (pyridin-3-yl)propanamide hydrochloride salt | 489.05 | 452.26 | 453 | V |
| 76 | 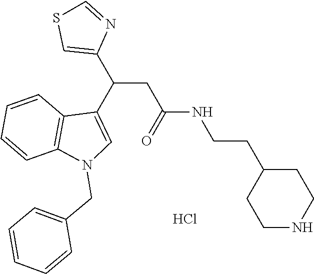 [SMILES: O=C(CC(c1cscn1)c1cn(Cc2ccccc2)c2ccccc12)NCCC1CCNCC1 ; Cl] | 3-(1-benzyl- 1H-indol-3-yl)- N-(2-(piperidin- 4-yl)ethyl)- 3-(thiazol-4-yl) propanamide hydrochloride salt | 509.11 | 472.23 | 473 | V |
| 77 | 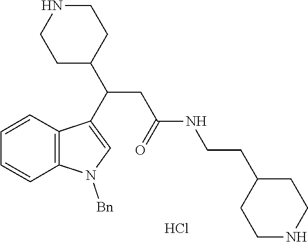 [SMILES: O=C(CC(c1cn(Cc2ccccc2)c2ccccc12)C1CCNCC1)NCCC1CCNCC1 ; Cl] | 3-(1-benzyl- 1H-indol-3-yl)- 3-(piperidin-4-yl)-N-(2- (piperidin-4- yl)ethyl)propanamide hydrochloride salt | 545.59 | 472.32 | 473 | V |
| 78 | 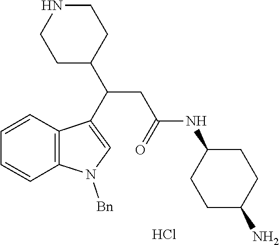 [SMILES: N[C@H]1CC[C@@H](NC(=O)CC(c2cn(Cc3ccccc3)c3ccccc23)C2CCNCC2)CC1 ; Cl] | N-((1s,4s)-4- aminocyclohexyl)-3-(1- benzyl-1H-indol-3-yl)-3- (piperidin-4- yl)propanamide hydrochloride salt | 531.56 | 458.30 | 459 | V |
| 79 | 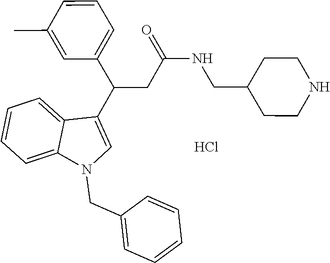 [SMILES: Cc1cccc(C(CC(=O)NCC2CCNCC2)c2cn(Cc3ccccc3)c3ccccc23)c1 ; Cl] | 3-(1-benzyl- 1H-indol-3-yl)- N-(piperidin-4- ylmethyl)-3- (m-tolyl)propanamide hydrochloride salt | 502.09 | 465.28 | 466 | V |
| 80 | 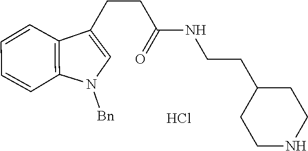 [SMILES: O=C(CCc1cn(Cc2ccccc2)c2ccccc12)NCCC1CCNCC1 ; Cl] | 3-(1-benzyl- 1H-indol-3-yl)- N-(piperidin-4-yl)-3-(m- tolyl)propanamide hydrochloride salt | 488.06 | 451.26 | 452 | V |
| 84 | 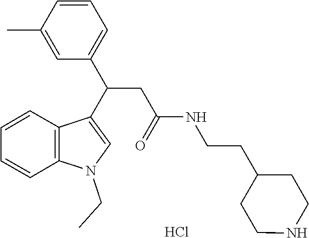 [SMILES: CCn1cc(C(CC(=O)NCCC2CCNCC2)c2cccc(C)c2)c2ccccc21 ; Cl] | 3-(1-ethyl-1H-indol-3-yl)- N-(2-(piperidin- 4-yl)ethyl)- 3-(m-tolyl)propanamide hydrochloride salt | 454.05 | 417.28 | 418 | V |
| 85 | 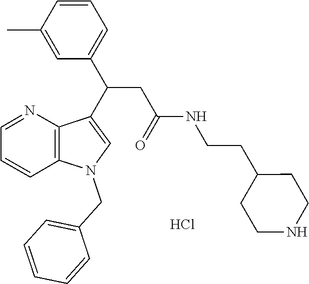 [SMILES: Cc1cccc(C(CC(=O)NCCC2CCNCC2)c2cn(Cc3ccccc3)c3cccnc23)c1 ; Cl] | 3-(1-benzyl-1H- pyrrolo[3,2- b]pyridin-3-yl)-N-(2- (piperidin-4-yl) ethyl)-3-(m- tolyl)propanamide hydrochloride salt | 517.1 | 480.29 | NA | X |
| 86 | 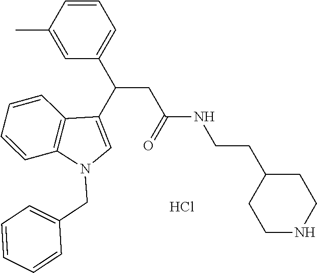 [SMILES: Cc1cccc(C(CC(=O)NCCC2CCNCC2)c2cn(Cc3ccccc3)c3ccccc23)c1 ; Cl] | 3-(1-benzyl-1H- indol-3-yl)- N-(2-(piperidin- 4-yl)ethyl)- 3-(m-tolyl)propanamide hydrochloride salt | 516.12 | 479.29 | NA | V |
| 87 | 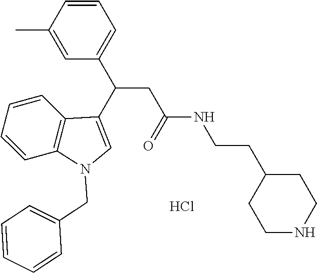 [SMILES: Cc1cccc(C(CC(=O)NCCC2CCNCC2)c2cn(Cc3ccccc3)c3ccccc23)c1 ; Cl] | 3-(1-benzyl-1H- indol-3-yl)- N-(2-(piperidin- 4-yl)ethyl)- 3-(m-tolyl) propanamide hydrochloride salt | 516.12 | 479.29 | NA | V |
| 88 | 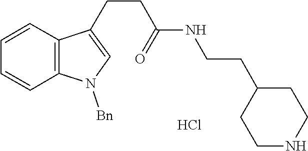 [SMILES: O=C(CCc1cn(Cc2ccccc2)c2ccccc12)NCCC1CCNCC1 ; Cl] | 3-(1-benzyl-1H- indol-3-yl)- N-(2-(piperidin-4- yl)ethyl)propanamide hydrochloride salt | 425.99 | 389.25 | 390 | V |
| 89 | 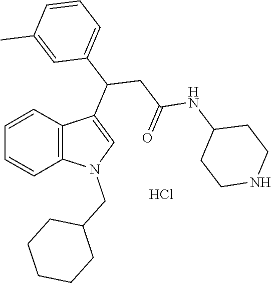 [SMILES: Cc1cccc(C(CC(=O)NC2CCNCC2)c2cn(CC3CCCCC3)c3ccccc23)c1 ; Cl] | 3-(1-(cyclohexylmethyl)- 1H-indol-3-yl)- N-(piperidin- 4-yl)-3-(m- tolyl)propanamide hydrochloride salt | 494.11 | 457.31 | 458 | V |
| 90 | 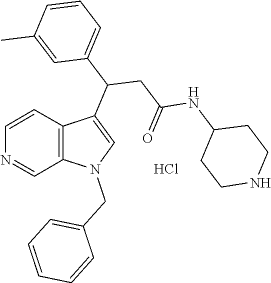 [SMILES: Cc1cccc(C(CC(=O)NC2CCNCC2)c2cn(Cc3ccccc3)c3cnccc23)c1 ; Cl] | 3-(1-benzyl-1H- pyrrolo[2,3- c]pyridin-3-yl)-N- (piperidin-4-yl)-3-(m- tolyl)propanamide hydrochloride salt | 489.05 | 452.26 | 453 | X |
| 91 | 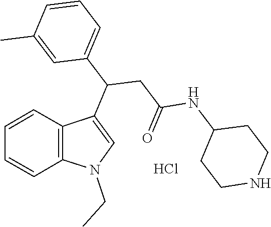 [SMILES: CCn1cc(C(CC(=O)NC2CCNCC2)c2cccc(C)c2)c2ccccc21 ; Cl] | 3-(1-ethyl-1H-indol-3-yl)- N-(piperidin-4-yl)-3-(m- tolyl)propanamide hydrochloride salt | 425.99 | 389.25 | 390 | V |
| 92 | 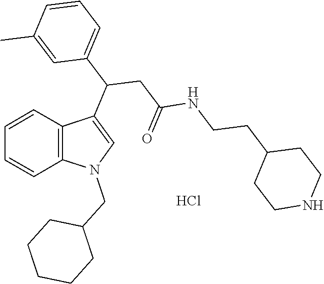 [SMILES: Cc1cccc(C(CC(=O)NCCC2CCNCC2)c2cn(CC3CCCCC3)c3ccccc23)c1 ; Cl] | 3-(1-(cyclohexylmethyl)- 1H-indol-3-yl)-N-(2- (piperidin-4-yl) ethyl)-3-(m- tolyl)propanamide hydrochloride salt | 522.16 | 485.34 | 486 | V |
| 93 | 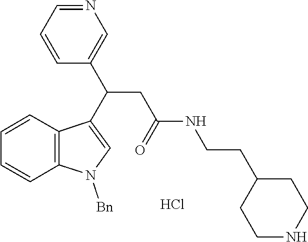 [SMILES: O=C(CC(c1cccnc1)c1cn(Cc2ccccc2)c2ccccc12)NCCC1CCNCC1 ; Cl] | 3-(1-benzyl- 1H-indol-3-yl)- N-(2-(piperidin- 4-yl)ethyl)- 3-(pyridin-3-yl) propanamide hydrochloride salt | 503.08 | 466.27 | 467 | V |
| 94 | 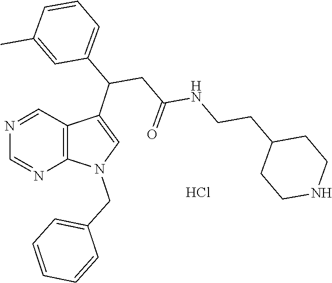 [SMILES: Cc1cccc(C(CC(=O)NCCC2CCNCC2)c2cn(Cc3ccccc3)c3ncncc23)c1 ; Cl] | 3-(7-benzyl-7H- pyrrolo[2,3- d]pyrimidin- 5-yl)-N-(2- (piperidin-4-yl) ethyl)-3-(m- tolyl)propanamide hydrochloride salt | 518.09 | 481.28 | 482 | X |
| 95 | 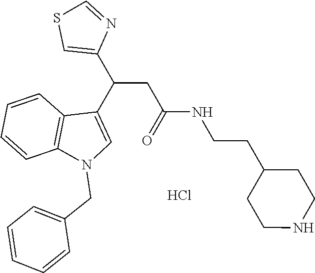 [SMILES: O=C(CC(c1cscn1)c1cn(Cc2ccccc2)c2ccccc12)NCCC1CCNCC1 ; Cl] | 3-(1-benzyl-1H- indol-3-yl)- N-(2-(piperidin- 4-yl)ethyl)- 3-(thiazol-4-yl) propanamide hydrochloride salt | 509.11 | 472.23 | NA | V |
| 96 | 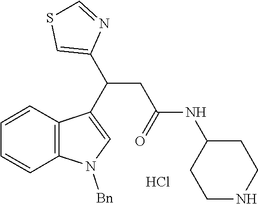 [SMILES: O=C(CC(c1cscn1)c1cn(Cc2ccccc2)c2ccccc12)NC1CCNCC1 ; Cl] | 3-(1-benzyl-1H- indol-3-yl)- N-(piperidin-4-yl)-3- (thiazol-4-yl) propanamide hydrochloride salt | 481.05 | 444.20 | 445 | V |
| 97 | 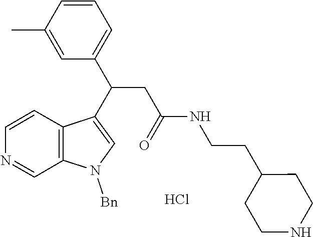 [SMILES: Cc1cccc(C(CC(=O)NCCC2CCNCC2)c2cn(Cc3ccccc3)c3cnccc23)c1 ; Cl] | 3-(1-benzyl-1H- pyrrolo[2,3- c]pyridin-3-yl)-N-(2- (piperidin-4-yl) ethyl)-3-(m- tolyl)propanamide hydrochloride salt | 517.1 | 480.29 | 482 | X |
| 98 | 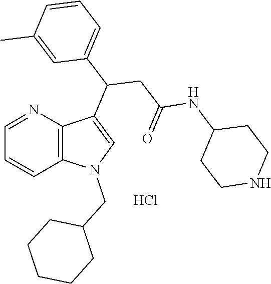 [SMILES: Cc1cccc(C(CC(=O)NC2CCNCC2)c2cn(CC3CCCCC3)c3cccnc23)c1 ; Cl] | 3-(1-(cyclohexylmethyl)- 1H-pyrrolo[3,2- b]pyridin-3- yl)-N-(piperidin-4- yl)-3-(m- tolyl)propanamide hydrochloride salt | 495.1 | 458.30 | 481 | X |
| 99 | 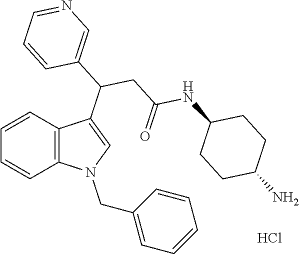 [SMILES: N[C@H]1CC[C@H](NC(=O)CC(c2cccnc2)c2cn(Cc3ccccc3)c3ccccc23)CC1 ; Cl] | N-((1R,4R)-4- aminocyclohexyl)-3-(1- benzyl-1H-indol-3-yl)-3- (pyridin-3-yl)propanamide hydrochloride salt | 489.05 | 452.26 | 453 | V |
| 100 | 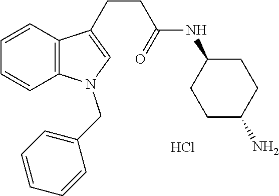 [SMILES: N[C@H]1CC[C@H](NC(=O)CCc2cn(Cc3ccccc3)c3ccccc23)CC1 ; Cl] | N-((1R,4R)-4- aminocyclohexyl)-3-(1- benzyl-1H-indol-3- yl)propanamide hydrochloride salt | 411.97 | 375.23 | 376 | V |
| 101 | 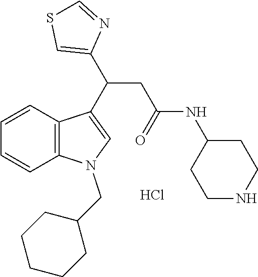 [SMILES: O=C(CC(c1cscn1)c1cn(CC2CCCCC2)c2ccccc12)NC1CCNCC1 ; Cl] | 3-(1-(cyclohexylmethyl)- 1H-indol-3-yl)- N-(piperidin- 4-yl)-3-(thiazol-4- yl)propanamide hydrochloride salt | 487.1 | 450.25 | 451 | V |
| 102 | 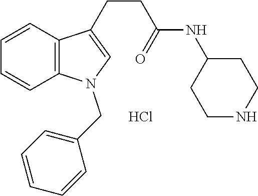 [SMILES: O=C(CCc1cn(Cc2ccccc2)c2ccccc12)NC1CCNCC1 ; Cl] | 3-(1-benzyl-1H- indol-3-yl)- N-(piperidin-4- yl)propanamide hydrochloride salt | 397.94 | 361.22 | 362 | V |
| 103 | 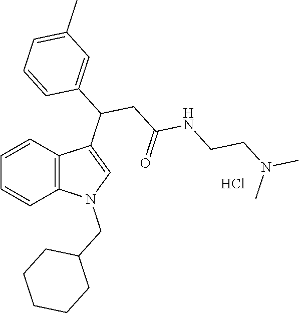 [SMILES: Cc1cccc(C(CC(=O)NCCN(C)C)c2cn(CC3CCCCC3)c3ccccc23)c1 ; Cl] | 3-(1-(cyclohexylmethyl)- 1H-indol-3-yl)-N-(2- (dimethylamino)ethyl)-3- (m-tolyl)propanamide hydrochloride salt | 482.1 | 445.31 | 446 | V |
| 104 | 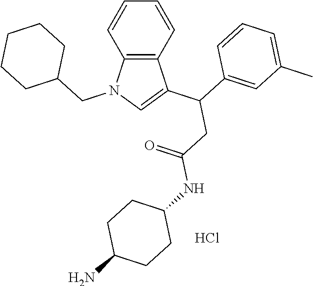 [SMILES: Cc1cccc(C(CC(=O)N[C@H]2CC[C@H](N)CC2)c2cn(CC3CCCCC3)c3ccccc23)c1 ; Cl] | N-((1R,4R)-4- aminocyclohexyl)-3-(1- (cyclohexylmethyl)-1H- indol-3-yl)-3-(m- tolyl)propanamide hydrochloride salt | 508.14 | 471.32 | 472 | V |
| 106 | 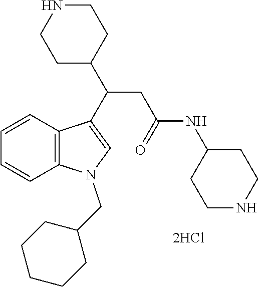 [SMILES: O=C(CC(c1cn(CC2CCCCC2)c2ccccc12)C1CCNCC1)NC1CCNCC1 ; "2HCl"] | 3-(1-(cyclohexylmethyl)- 1H-indol-3-yl)-N,3- di(piperidin-4- yl)propanamide hydrochloride salt | 523.58 | 450.34 | 451 | V |
| 107 | 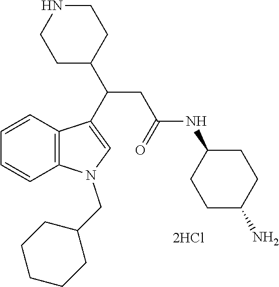 [SMILES: N[C@H]1CC[C@H](NC(=O)CC(c2cn(CC3CCCCC3)c3ccccc23)C2CCNCC2)CC1 ; "2HCl"] | N-((1r,4r)-4- aminocyclohexyl)-3-(1- (cyclohexylmethyl)-1H- indol-3-yl)-3-(piperidin-4- yl)propanamide hydrochloride salt | 537.61 | 464.35 | 465 | V |
| 108 | 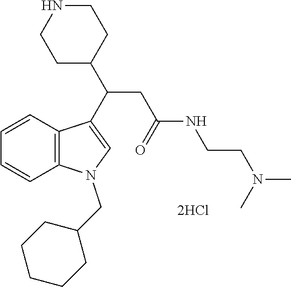 [SMILES: CN(C)CCNC(=O)CC(c1cn(CC2CCCCC2)c2ccccc12)C1CCNCC1 ; "2HCl"] | 3-(1-(cyclohexylmethyl)- 1H-indol-3-yl)-N-(2- (dimethylamino)ethyl)-3- (piperidin-4-yl) propanamide hydrochloride salt | 511.57 | 438.34 | 439 | V |
| 109 | 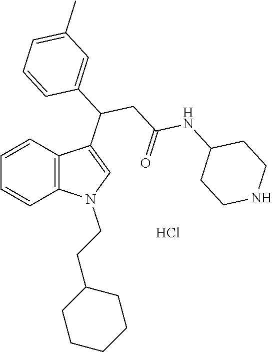 [SMILES: Cc1cccc(C(CC(=O)NC2CCNCC2)c2cn(CCC3CCCCC3)c3ccccc23)c1 ; Cl] | 3-(1-(2-cyclohexylethyl)- 1H-indol-3-yl)- N-(piperidin- 4-yl)-3-(m- tolyl)propanamide hydrochloride salt | 508.14 | 471.32 | 472 | V |
| 110 | 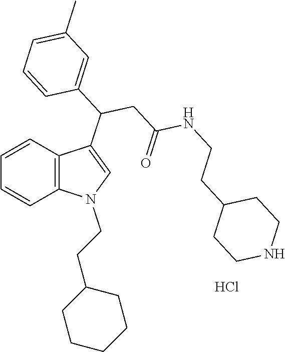 [SMILES: Cc1cccc(C(CC(=O)NCCC2CCNCC2)c2cn(CCC3CCCCC3)c3ccccc23)c1 ; Cl] | 3-(1-(2-cyclohexylethyl)- 1H-indol-3-yl)-N-(2- (piperidin-4-yl) ethyl)-3-(m- tolyl)propanamide hydrochloride salt | 536.19 | 499.36 | 500 | V |
| 111 | 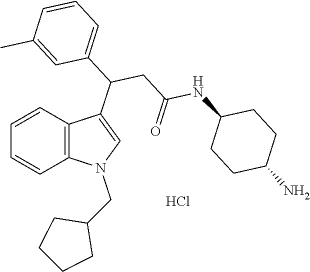 [SMILES: Cc1cccc(C(CC(=O)N[C@H]2CC[C@H](N)CC2)c2cn(CC3CCCC3)c3ccccc23)c1 ; Cl] | N-((1R,4R)-4- aminocyclohexyl)-3-(1- (cyclopentylmethyl)-1H- indol-3-yl)-3-(m- tolyl)propanamide hydrochloride salt | 494.11 | 457.31 | 458 | V |
| 112 | 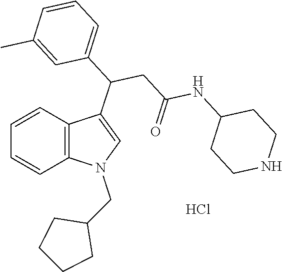 [SMILES: Cc1cccc(C(CC(=O)NC2CCNCC2)c2cn(CC3CCCC3)c3ccccc23)c1 ; Cl] | 3-(1-(cyclopentylmethyl)- 1H-indol-3-yl)- N-(piperidin- 4-yl)-3-(m- tolyl)propanamide hydrochloride salt | 480.08 | 443.29 | 444 | V |
| 113 | 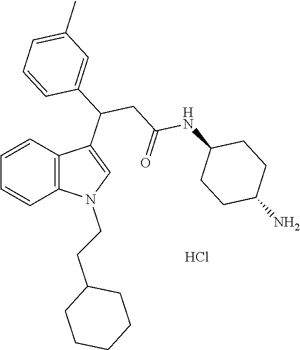 [SMILES: Cc1cccc(C(CC(=O)N[C@H]2CC[C@H](N)CC2)c2cn(CCC3CCCCC3)c3ccccc23)c1 ; Cl] | N-((1R,4R)-4- aminocyclohexyl)- 3-(1-(2- cyclohexylethyl)- 1H-indol- 3-yl)-3-(m- tolyl)propanamide hydrochloride salt | 522.16 | 485.34 | 486 | V |
| 114 | 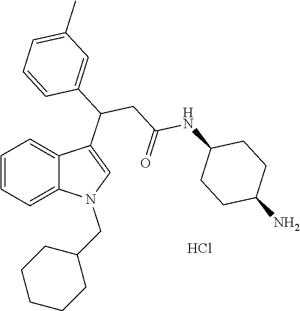 [SMILES: Cc1cccc(C(CC(=O)N[C@H]2CC[C@@H](N)CC2)c2cn(CC3CCCCC3)c3ccccc23)c1 ; Cl] | N-((1s,4s)-4- aminocyclohexyl)-3-(1- (cyclohexylmethyl)-1H- indol-3-yl)-3-(m- tolyl)propanamide hydrochloride salt | 508.14 | 471.32 | 472 | V |
| 115 | 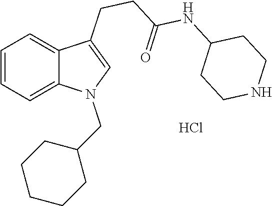 [SMILES: O=C(CCc1cn(CC2CCCCC2)c2ccccc12)NC1CCNCC1 ; Cl] | 3-(1-(cyclohexylmethyl)- 1H-indol-3-yl)- N-(piperidin- 4-yl)propanamide hydrochloride salt | 403.99 | 367.26 | 368 | V |
| 116 | 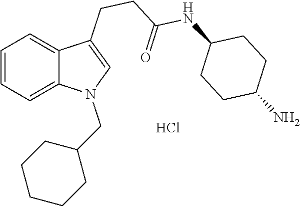 [SMILES: N[C@H]1CC[C@H](NC(=O)CCc2cn(CC3CCCCC3)c3ccccc23)CC1 ; Cl] | N-((1r,4r)-4- aminocyclohexyl)-3-(1- (cyclohexylmethyl)-1H- indol-3-yl)propanamide hydrochloride salt | 418.02 | 381.28 | 382 | V |
| 117 | 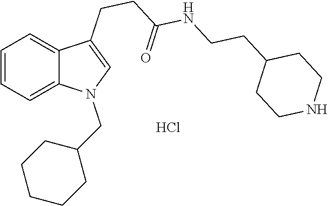 [SMILES: O=C(CCc1cn(CC2CCCCC2)c2ccccc12)NCCC1CCNCC1 ; Cl] | 3-(1-(cyclohexylmethyl)- 1H-indol-3-yl)-N-(2- (piperidin-4- yl)ethyl)propanamide hydrochloride salt | 432.04 | 395.29 | 396 | V |
| 118 | 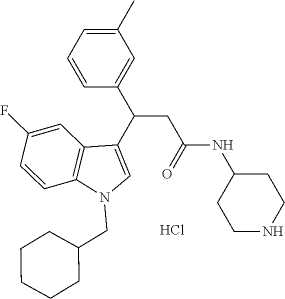 [SMILES: Cc1cccc(C(CC(=O)NC2CCNCC2)c2cn(CC3CCCCC3)c3ccc(F)cc23)c1 ; Cl] | 3-(1- (cyclohexylmethyl)-5- fluoro-1H-indol-3-yl)-N- (piperidin-4-yl)-3-(m- tolyl)propanamide hydrochloride salt | 512.1 | 475.3 | 476 | V |
| 119 | 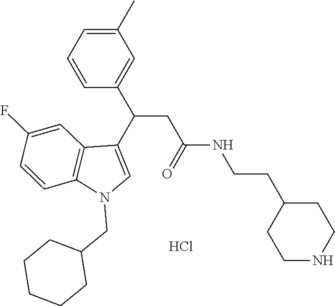 [SMILES: Cc1cccc(C(CC(=O)NCCC2CCNCC2)c2cn(CC3CCCCC3)c3ccc(F)cc23)c1 ; Cl] | 3-(1- (cyclohexylmethyl)-5- fluoro-1H-indol- 3-yl)-N-(2- (piperidin-4-yl) ethyl)-3-(m- tolyl)propanamide hydrochloride salt | 540.15 | 503.33 | 504 | V |
| 120 | 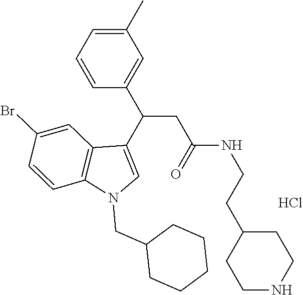 [SMILES: Cc1cccc(C(CC(=O)NCCC2CCNCC2)c2cn(CC3CCCCC3)c3ccc(Br)cc23)c1 ; Cl] | 3-(5-bromo-1- (cyclohexylmethyl)-1H- indol-3-yl)-N- (2-(piperidin- 4-yl)ethyl)-3-(m- tolyl)propanamide hydrochloride salt | 601.06 | 563.25 | 566 | V |
| 121 | 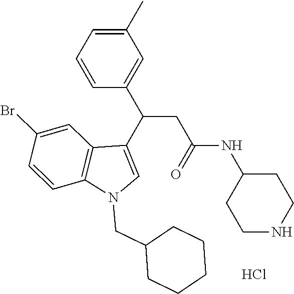 [SMILES: Cc1cccc(C(CC(=O)NC2CCNCC2)c2cn(CC3CCCCC3)c3ccc(Br)cc23)c1 ; Cl] | 3-(5-bromo-1- (cyclohexylmethyl)-1H- indol-3-yl)-N- (piperidin-4- yl)-3-(m-tolyl) propanamide hydrochloride salt | 573.01 | 535.22 | 538 | V |
| 122 | 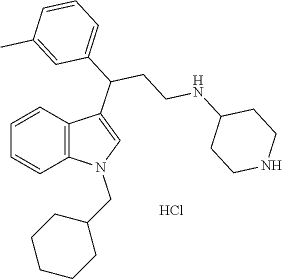 [SMILES: Cc1cccc(C(CCNC2CCNCC2)c2cn(CC3CCCCC3)c3ccccc23)c1 ; Cl] | N-(3-(1- (cyclohexylmethyl)- 1H-indol-3-yl)-3-(m- tolyl)propyl)piperidin-4- amine hydrochloride salt | 480.13 | 443.33 | NA | V |
| 123 | 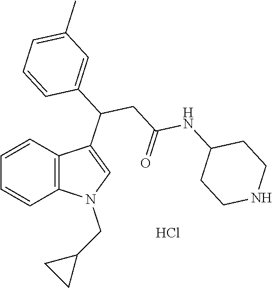 [SMILES: Cc1cccc(C(CC(=O)NC2CCNCC2)c2cn(CC3CC3)c3ccccc23)c1 ; Cl] | 3-(1- (cyclopropylmethyl)- 1H-indol-3-yl)- N-(piperidin- 4-yl)-3-(m- tolyl)propanamide hydrochloride salt | 452.03 | 415.26 | 416 | V |
| 124 | 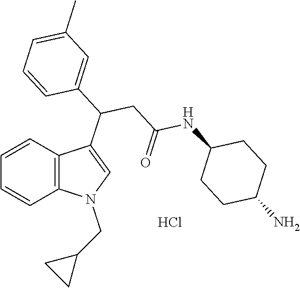 [SMILES: Cc1cccc(C(CC(=O)N[C@H]2CC[C@H](N)CC2)c2cn(CC3CC3)c3ccccc23)c1 ; Cl] | N-((1R,4R)-4- aminocyclohexyl)-3-(1- (cyclopropylmethyl)-1H- indol-3-yl)-3-(m- tolyl)propanamide hydrochloride salt | 466.06 | 429.28 | 430 | V |
| 125 | 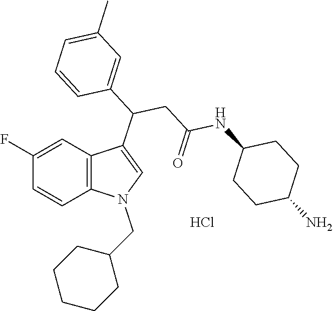 [SMILES: Cc1cccc(C(CC(=O)N[C@H]2CC[C@H](N)CC2)c2cn(CC3CCCCC3)c3ccc(F)cc23)c1 ; Cl] | N-((1R,4R)-4- aminocyclohexyl)-3-(1- (cyclohexylmethyl)-5- fluoro-1H- indol-3-yl)-3-(m- tolyl)propanamide hydrochloride salt | 526.13 | 489.2 | 490 | V |
| 126 | 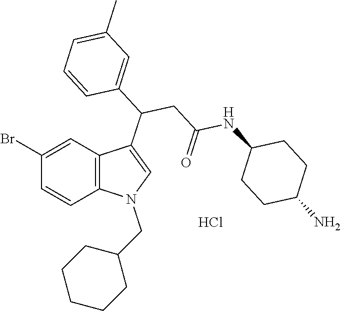 [SMILES: Cc1cccc(C(CC(=O)N[C@H]2CC[C@H](N)CC2)c2cn(CC3CCCCC3)c3ccc(Br)cc23)c1 ; Cl] | N-((1R,4R)-4- aminocyclohexyl)-3-(5- bromo-1- (cyclohexylmethyl)-1H- indol-3-yl)-3-(m- tolyl)propanamide hydrochloride salt | 587.03 | 549.24 | | V |
| 127 | 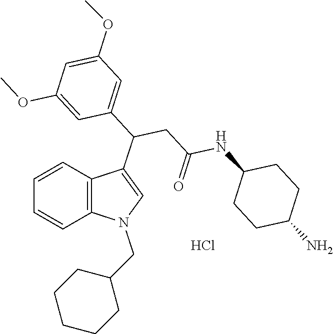 [SMILES: COc1cc(OC)cc(C(CC(=O)N[C@H]2CC[C@H](N)CC2)c2cn(CC3CCCCC3)c3ccccc23)c1 ; Cl] | N-((1R,4R)-4- aminocyclohexyl)-3-(1- (cyclohexylmethyl)-1H- indol-3-yl)-3-(3,5- dimethoxyphenyl) propanamide hydrochloride salt | 554.16 | 517.33 | 518 | V |
| 128 | 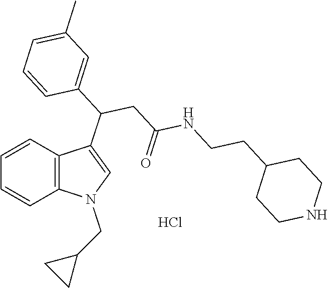 [SMILES: Cc1cccc(C(CC(=O)NCCC2CCNCC2)c2cn(CC3CC3)c3ccccc23)c1 ; Cl] | 3-(1-(cyclopropylmethyl)- 1H-indol-3-yl)-N-(2- (piperidin-4-yl)ethyl)- 3-(m-tolyl) propanamide hydrochloride salt | 480.08 | 443.29 | 444 | V |
| 129 | 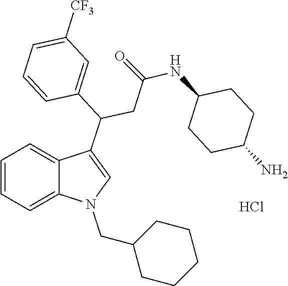 [SMILES: N[C@H]1CC[C@H](NC(=O)CC(c2cccc(C(F)(F)F)c2)c2cn(CC3CCCCC3)c3ccccc23)CC1 ; Cl] | N-((1R,4R)-4- aminocyclohexyl)-3-(1- (cyclohexylmethyl)-1H- indol-3-yl)-3-(3- (trifluoromethyl)phenyl) propanamide hydrochloride salt | 562.11 | 525.3 | NA | V |
| 130 | 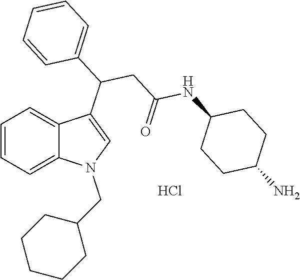 [SMILES: N[C@H]1CC[C@H](NC(=O)CC(c2ccccc2)c2cn(CC3CCCCC3)c3ccccc23)CC1 ; Cl] | N-((1R,4R)-4- aminocyclohexyl)-3-(1- (cyclohexylmethyl)-1H- indol-3-yl)-3- phenylpropanamide hydrochloride salt | 494.11 | 457.31 | 458 | V |
| 131 | 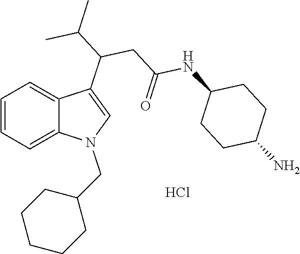 [SMILES: CC(C)C(CC(=O)N[C@H]1CC[C@H](N)CC1)c1cn(CC2CCCCC2)c2ccccc12 ; Cl] | N-((1R,4R)-4- aminocyclohexyl)-3-(1- (cyclohexylmethyl)-1H- indol-3-yl)-4- methylpentanamide hydrochloride salt | 460.09 | 423.32 | 424 | V |
| 132 | 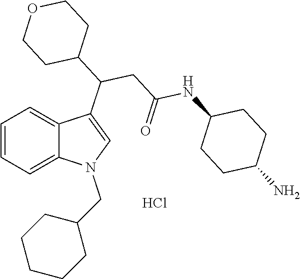 [SMILES: N[C@H]1CC[C@H](NC(=O)CC(c2cn(CC3CCCCC3)c3ccccc23)C2CCOCC2)CC1 ; Cl] | N-((1R,4R)-4- aminocyclohexyl)-3-(1- (cyclohexylmethyl)-1H- indol-3-yl)-3-(tetrahydro- 2H-pyran-4- yl)propanamide hydrochloride salt | 502.13 | 465.34 | 466 | V |
| 133 | 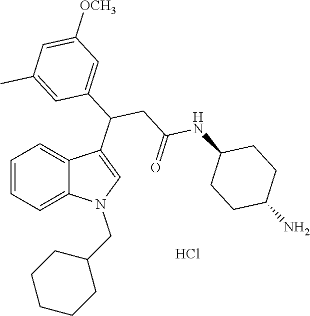 [SMILES: COc1cc(C)cc(C(CC(=O)N[C@H]2CC[C@H](N)CC2)c2cn(CC3CCCCC3)c3ccccc23)c1 ; Cl] | N-((1R,4R)-4- aminocyclohexyl)-3-(1- (cyclohexylmethyl)-1H- indol-3-yl)-3- (3-methoxy-5- methylphenyl) propanamide hydrochloride salt | 538.16 | 501.34 | 502 | V |
| 134 | 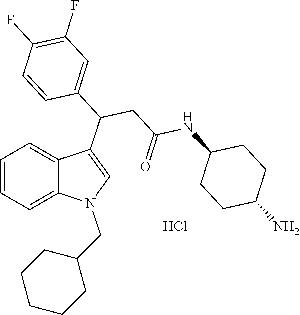 [SMILES: N[C@H]1CC[C@H](NC(=O)CC(c2ccc(F)c(F)c2)c2cn(CC3CCCCC3)c3ccccc23)CC1 ; Cl] | N-((1R,4R)-4- aminocyclohexyl)-3-(1- (cyclohexylmethyl)-1H- indol-3-yl)-3-(3,4- difluorophenyl )propanamide hydrochloride salt | 530.09 | 493.29 | 494 | V |
| 135 | 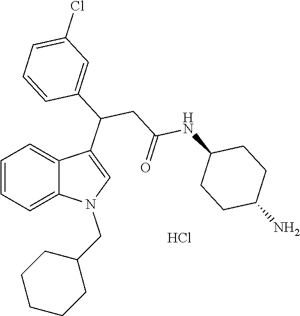 [SMILES: N[C@H]1CC[C@H](NC(=O)CC(c2cccc(Cl)c2)c2cn(CC3CCCCC3)c3ccccc23)CC1 ; Cl] | N-((1r,4r)-4- aminocyclohexyl)-3-(3- chlorophenyl)-3-(1- (cyclohexylmethyl)-1H- indol-3-yl)propanamide hydrochloride salt | 528.56 | 491.27 | 492 | V |
| 136 | 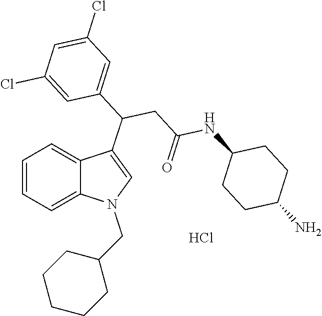 [SMILES: N[C@H]1CC[C@H](NC(=O)CC(c2cc(Cl)cc(Cl)c2)c2cn(CC3CCCCC3)c3ccccc23)CC1 ; Cl] | N-((1r,4r)-4- aminocyclohexyl)-3-(1- (cyclohexylmethyl)-1H- indol-3-yl)-3-(3,5- dichlorophenyl) propanamide hydrochloride salt | 563 | 525.23 | 526 | V |
| 137 | 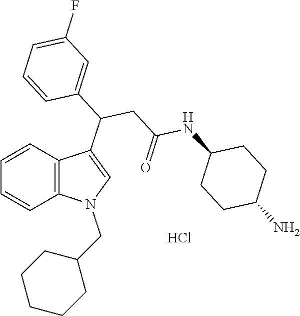 [SMILES: N[C@H]1CC[C@H](NC(=O)CC(c2cccc(F)c2)c2cn(CC3CCCCC3)c3ccccc23)CC1 ; Cl] | N-((1R,4R)-4- aminocyclohexyl)-3-(1- (cyclohexylmethyl)-1H- indol-3-yl)-3-(3- fluorophenyl) propanamide hydrochloride salt | 512.1 | 475.3 | 476 | V |
| 138 | 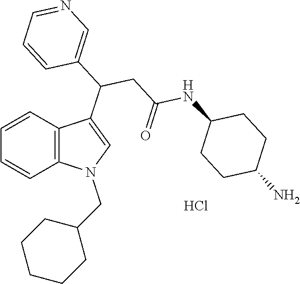 [SMILES: N[C@H]1CC[C@H](NC(=O)CC(c2cccnc2)c2cn(CC3CCCCC3)c3ccccc23)CC1 ; Cl] | N-((1R,4R)-4- aminocyclohexyl)-3-(1- (cyclohexylmethyl)-1H- indol-3-yl)-3-(pyridin-3- yl)propanamide hydrochloride salt | 495.1 | 458.30 | 459 | V |
| 139 | 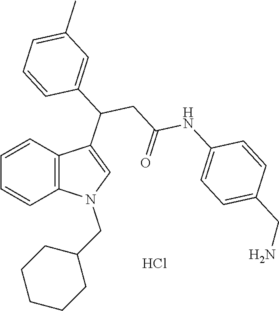 [SMILES: Cc1cccc(C(CC(=O)Nc2ccc(CN)cc2)c2cn(CC3CCCCC3)c3ccccc23)c1 ; Cl] | N-(4-(aminomethyl) phenyl)-3-(1- (cyclohexylmethyl)- 1H-indol-3-yl)-3-(m- tolyl)propanamide hydrochloride salt | 516.12 | 479.29 | 481 | V |
| 140 | 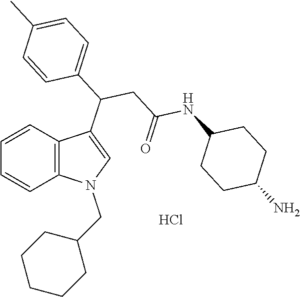 | N-((1S,4S)-4- aminocyclohexyl)-3-(1- (cyclohexylmethyl)-1H- indol-3-yl)-3-(p- tolyl)propanamide hydrochloride salt | 508.14 | 471.32 | 472 | V |
| 142 | 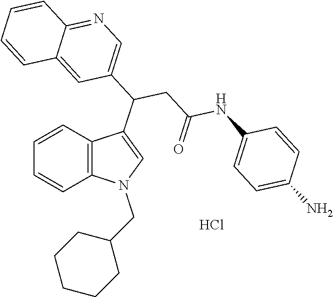 | N-((1S,4S)-4- aminocyclohexyl)-3-(1- (cyclohexylmethyl)-1H- indol-3-yl)-3-(quinolin-3- yl)propanamide hydrochloride salt | 545.16 | 508.32 | 509 | V |
| 143 | 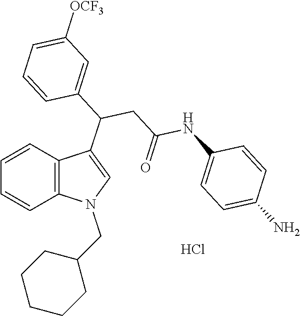 | N-((1S,4S)-4- aminocyclohexyl)-3-(1- (cyclohexylmethyl)-1H- indol-3-yl)-3-(3- (trifluoromethoxy)phenyl) propanamide hydrochloride salt | 578.11 | 541.29 | 542 | V |
| 144 | 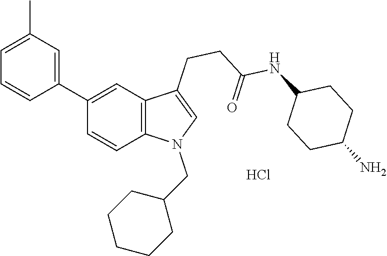 | N-((1s,4s)-4- aminocyclohexyl)-3-(1- (cyclohexylmethyl)-5-(m- tolyl)-1H-indol-3- yl)propanamide hydrochloride salt | 508.14 | 471.32 | 472 | V |
| 145 | 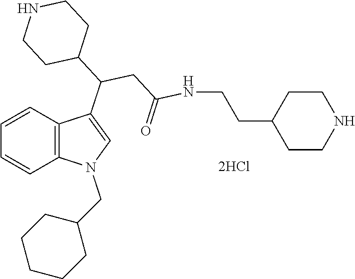 [SMILES: O=C(CC(c1cn(CC2CCCCC2)c2ccccc12)C1CCNCC1)NCCC1CCNCC1 ; "2HCl"] | 3-(1-(cyclohexylmethyl)- 1H-indol-3-yl)- 3-(piperidin-4-yl)- N-(2-(piperidin-4- yl)ethyl)propanamide hydrochloride salt | 551.63 | 478.37 | 479 | V |
| 146 | 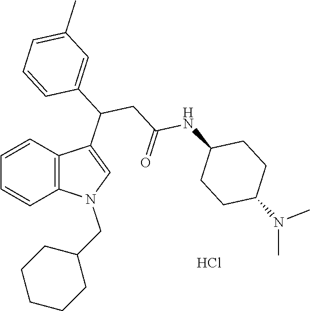 [SMILES: Cc1cccc(C(CC(=O)N[C@H]2CC[C@H](N(C)C)CC2)c2cn(CC3CCCCC3)c3ccccc23)c1 ; Cl] | 3-(1-(cyclohexylmethyl)- 1H-indol-3-yl)-N- ((1R,4R)- 4- (dimethylamino) cyclohexyl)- 3-(m-tolyl)propanamide hydrochloride salt | 536.19 | 499.36 | 500 | V |
| 149 | 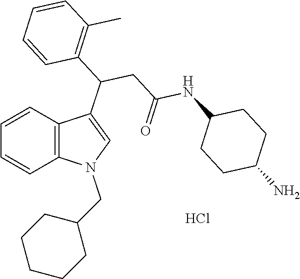 | N-((1S,4S)-4- aminocyclohexyl)-3-(1- (cyclohexylmethyl)-1H- indol-3-yl)-3-(o- tolyl)propanamide hydrochloride salt | 508.14 | 471.32 | NA | V |
| 150 | 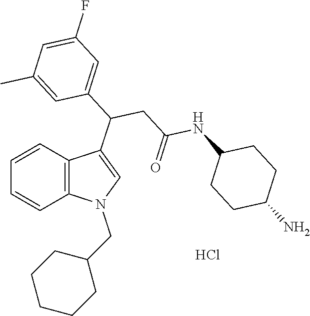 | N-((1S,4S)-4- aminocyclohexyl)-3-(1- (cyclohexylmethyl)-1H- indol-3-yl)-3-(3-fluoro-5- methylphenyl) propanamide hydrochloride salt | 526.13 | 489.32 | 502 | V |
| 151 | 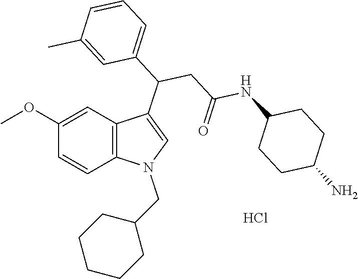 | N-((1S,4S)-4- aminocyclohexyl)-3-(1- (cyclohexylmethyl)-5- methoxy-1H-indol-3-yl)- 3-(m-tolyl)propanamide hydrochloride salt | 538.16 | 501.34 | 490 | V |
| 153 | 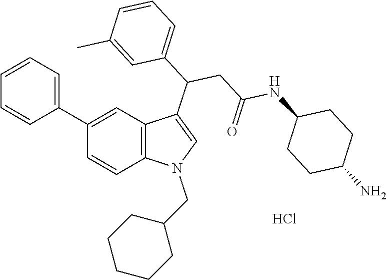 [SMILES: Cc1cccc(C(CC(=O)N[C@H]2CC[C@H](N)CC2)c2cn(CC3CCCCC3)c3ccc(-c4ccccc4)cc23)c1 ; Cl] | N-((1R,4R)-4- aminocyclohexyl)-3-(1- (cyclohexylmethyl)-5- phenyl-1H-indol- 3-yl)-3-(m- tolyl)propanamide hydrochloride salt | 584.23 | 547.36 | 548.6 | V |
| 154 | 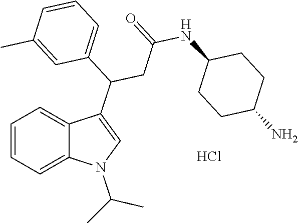 [SMILES: Cc1cccc(C(CC(=O)N[C@H]2CC[C@H](N)CC2)c2cn(C(C)C)c3ccccc23)c1 ; Cl] | N-((1R,4R)-4- aminocyclohexyl)-3-(1- isopropyl-1H- indol-3-yl)-3- (m-tolyl)propanamide hydrochloride salt | 454.05 | 417.28 | 418 | V |
| 155 | 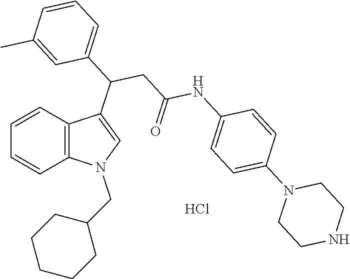 [SMILES: Cc1cccc(C(CC(=O)Nc2ccc(N3CCNCC3)cc2)c2cn(CC3CCCCC3)c3ccccc23)c1 ; Cl] | 3-(1-(cyclohexylmethyl)- 1H-indol-3-yl)-N-(4- (piperazin-1-yl)phenyl)-3- (m-tolyl)propanamide hydrochloride salt | 571.2 | 534.34 | 534 | V |
| 156 | 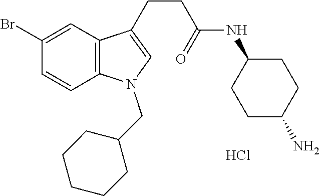 [SMILES: N[C@H]1CC[C@H](NC(=O)CCc2cn(CC3CCCCC3)c3ccc(Br)cc23)CC1 ; Cl] | N-((1R,4R)-4- aminocyclohexyl)-3-(5- bromo-1- (cyclohexylmethyl)-1H- indol-3-yl)propanamide hydrochloride salt | 496.91 | 459.19 | NA | V |
| 157 | 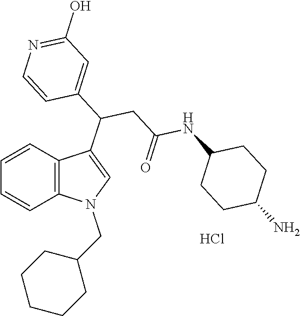 | N-((1S,4S)-4- aminocyclohexyl)-3-(1- (cyclohexylmethyl)-1H- indol-3-yl)-3-(2- hydroxypyridin-4- yl)propanamide hydrochloride salt | 511.1 | 474.30 | 475 | V |
| 158 | 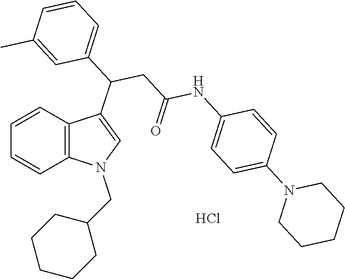 [SMILES: Cc1cccc(C(CC(=O)Nc2ccc(N3CCCCC3)cc2)c2cn(CC3CCCCC3)c3ccccc23)c1 ; Cl] | 3-(1-(cyclohexylmethyl)- 1H-indol-3-yl)-N-(4- (piperidin-1-yl)phenyl)-3- (m-tolyl)propanamide hydrochloride salt | 570.21 | 533.34 | 534 | V |
| 159 | 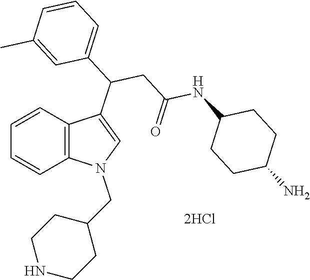 | N-((1S,4S)-4- aminocyclohexyl)-3-(1- (piperidin-4-ylmethyl)- 1H-indol-3-yl)-3-(m- tolyl)propanamide hydrochloride salt | 545.59 | 472.32 | | V |
| 160 | 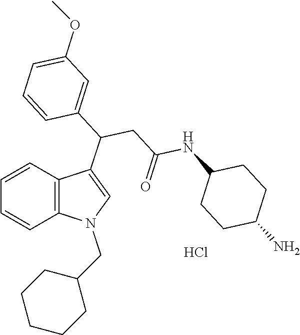 | N-((1S,4S)-4- aminocyclohexyl)-3-(1- (cyclohexylmethyl)-1H- indol-3-yl)-3-(3- methoxyphenyl) propanamide hydrochloride salt | 524.14 | 487.32 | 488 | V |
| 161 | 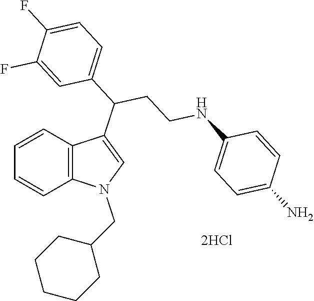 | (1S,4S)-N1-(3-(1- (cyclohexylmethyl)-1H- indol-3-yl)-3-(3,4- difluorophenyl)propyl) cyclohexane- 1,4-diamine hydrochloride salt | 552.57 | 479.31 | 480 | I |
| 162 | 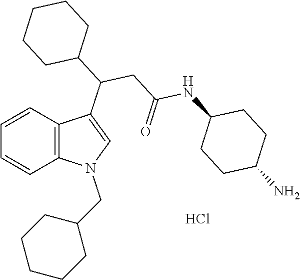 [SMILES: N[C@H]1CC[C@H](NC(=O)CC(c2cn(CC3CCCCC3)c3ccccc23)C2CCCCC2)CC1 ; Cl] | N-((1R,4R)-4- aminocyclohexyl)-3- cyclohexyl-3-(1- (cyclohexylmethyl)-1H- indol-3-yl)propanamide hydrochloride salt | 500.16 | 463.36 | 464 | V |
| 163 | 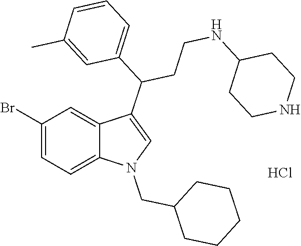 [SMILES: Cc1cccc(C(CCNC2CCNCC2)c2cn(CC3CCCCC3)c3ccc(Br)cc23)c1 ; Cl] | N-(3-(5-bromo-1- (cyclohexylmethyl)-1H- indol-3-yl)-3-(m- tolyl)propyl)piperidin-4- amine hydrochloride salt | 595.48 | 521.34 | 524 | II |
| 164 | 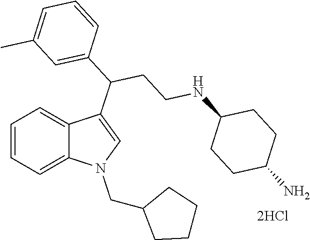 [SMILES: Cc1cccc(C(CCN[C@H]2CC[C@H](N)CC2)c2cn(CC3CCCC3)c3ccccc23)c1 ; "2HCl"] | (1R,4R)-N1-(3-(1- (cyclopentylmethyl)-1H- indol-3-yl)-3-(m- tolyl)propyl)cyclohexane- 1,4-diamine hydrochloride salt | 516.59 | 443.33 | 444 | II |
| 165 | 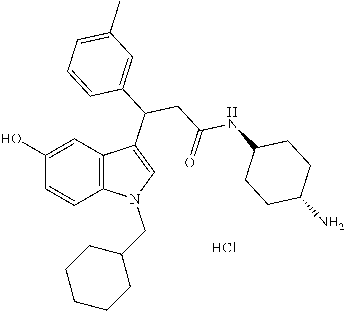 | N-((1S,4S)-4- aminocyclohexyl)-3-(1- (cyclohexylmethyl)-5- hydroxy-1H- indol-3-yl)-3- (m-tolyl)propanamide hydrochloride salt | 524.14 | 487.32 | 489 | V |
| 166 | 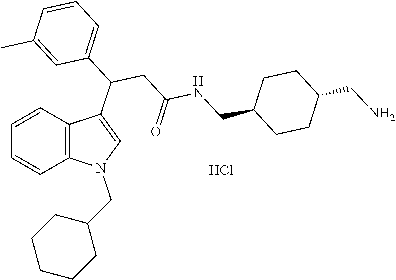 [SMILES: Cc1cccc(C(CC(=O)NC[C@H]2CC[C@H](CN)CC2)c2cn(CC3CCCCC3)c3ccccc23)c1 ; Cl] | N-(((1S,4S)-4- (aminomethyl) cyclohexyl) methyl)-3-(1- (cyclohexylmethyl)-1H- indol-3-yl)-3-(m- tolyl)propanamide hydrochloride salt | 536.19 | 499.36 | 500 | V |
| 167 | 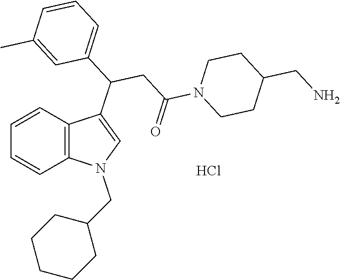 [SMILES: Cc1cccc(C(CC(=O)N2CCC(CN)CC2)c2cn(CC3CCCCC3)c3ccccc23)c1 ; Cl] | 1-(4- (aminomethyl)piperidin- 1-yl)-3-(1- (cyclohexylmethyl)-1H- indol-3-yl)-3-(m- tolyl)propan-1-one hydrochloride salt | 508.14 | 471.32 | 472 | V |
| 168 | 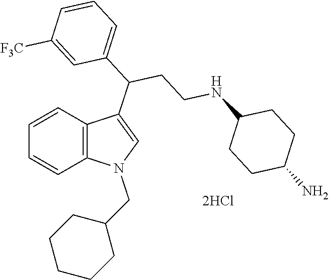 | (1S,4S)-N1-(3-(1- (cyclohexylmethyl)-1H- indol-3-yl)-3-(3- (trifluoromethyl)phenyl) propyl)cyclohexane-1,4- diamine hydrochloride salt | 584.59 | 511.32 | 512 | II |
| 169 | 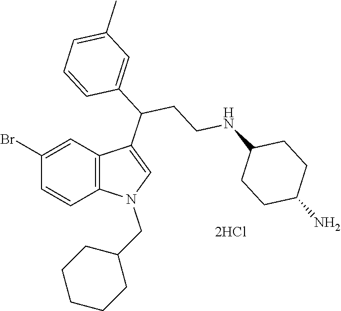 | (1S,4S)-N1-(3- (5-bromo-1- (cyclohexylmethyl)- 1H-indol-3-yl)-3-(m- tolyl)propyl)cyclohexane- 1,4-diamine hydrochloride salt | 609.51 | 535.26 | 538 | II |
| 170 | 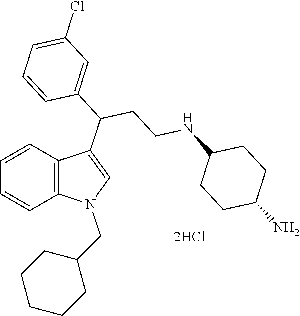 | (1S,4S)-N1-(3-(3- chlorophenyl)-3-(1- (cyclohexylmethyl)-1H- indol-3-yl)propyl) cyclohexane-1,4- diamine hydrochloride salt | 551.03 | 477.29 | NA | II |
| 171 | 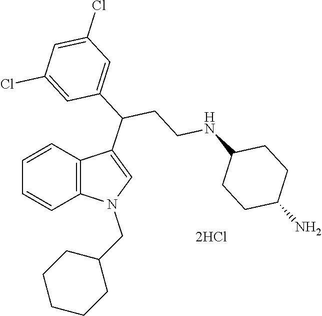 | (1S,4S)-N1-(3-(1- (cyclohexylmethyl)-1H- indol-3-yl)-3-(3,5- dichlorophenyl)propyl) cyclohexane-1,4-diamine hydrochloride salt | 585.48 | 511.25 | 512 | II |
| 172 | 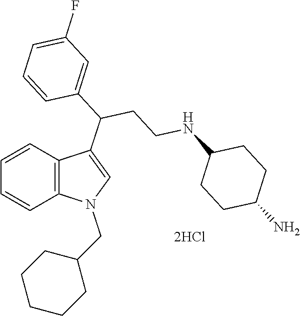 | (1S,4S)-N1-(3-(1- (cyclohexylmethyl)-1H- indol-3-yl)-3-(3- fluorophenyl)propyl) cyclohexane-1,4-diamine hydrochloride salt | 534.58 | 461.32 | 462 | II |
| 173 | 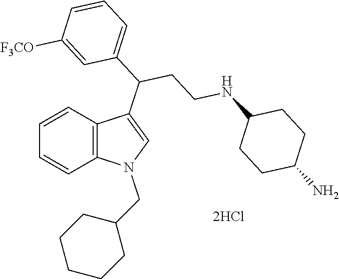 | (1S,4S)-N1-(3-(1- (cyclohexylmethyl)-1H- indol-3-yl)-3-(3- (trifluoromethoxy)phenyl) propyl)cyclohexane-1,4- diamine hydrochloride salt | 600.59 | 527.31 | 528 | II |
| 174 | 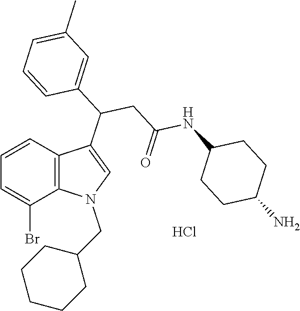 | N-((1S,4S)-4- aminocyclohexyl)-3-(7- bromo-1- (cyclohexylmethyl)-1H- indol-3-yl)-3-(m- tolyl)propanamide hydrochloride salt | 587.03 | 549.24 | 550 | V |
| 175 | 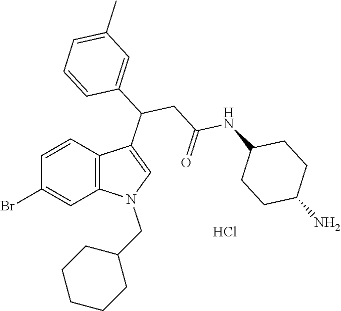 | N-((1S,4S)-4- aminocyclohexyl)-3-(6- bromo-1- (cyclohexylmethyl)-1H- indol-3-yl)-3-(m- tolyl)propanamide hydrochloride salt | 587.03 | 549.24 | 550 | V |
| 177 | 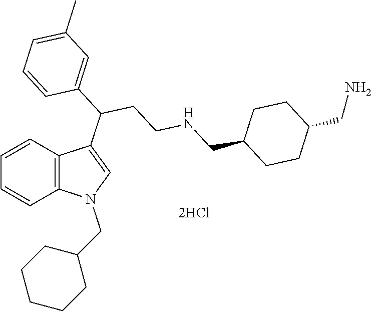 [SMILES: Cc1cccc(C(CCNC[C@H]2CC[C@H](CN)CC2)c2cn(CC3CCCCC3)c3ccccc23)c1 ; "2HCl"] | N-(((1R,4R)-4- (aminomethyl)cyclohexyl) methyl)-3-(1- (cyclohexylmethyl)-1H- indol-3-yl)-3-(m- tolyl)propan-1-amine hydrochloride salt | 558.67 | 485.38 | 486 | II |
| 178 | 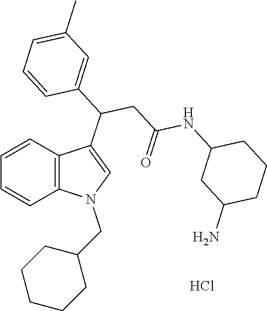 [SMILES: Cc1cccc(C(CC(=O)NC2CCCC(N)C2)c2cn(CC3CCCCC3)c3ccccc23)c1 ; Cl] | N-(3-aminocyclohexyl)-3- (1-(cyclohexylmethyl)- 1H-indol-3-yl)-3-(m- tolyl)propanamide hydrochloride salt | 508.14 | 471.32 | 472 | V |
| 179 | 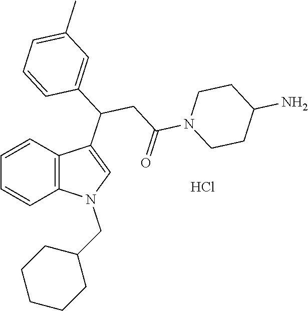 [SMILES: Cc1cccc(C(CC(=O)N2CCC(N)CC2)c2cn(CC3CCCCC3)c3ccccc23)c1 ; Cl] | 1-(4- aminopiperidin- 1-yl)-3-(1- (cyclohexylmethyl)- 1H-indol-3-yl)-3-(m- tolyl)propan-1-one hydrochloride salt | 494.11 | 457.31 | 458 | V |
| 180 | 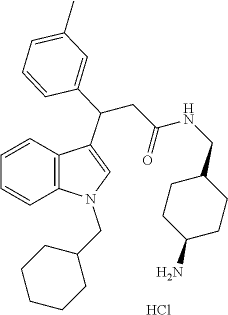 | N-(((1R,4R)-4- aminocyclohexyl) methyl)-3-(1- (cyclohexylmethyl)- 1H- indol-3-yl)-3-(m- tolyl)propanamide hydrochloride salt | 522.16 | 485.34 | 486 | V |
| 181 | 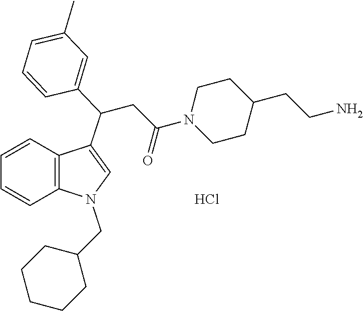 [SMILES: Cc1cccc(C(CC(=O)N2CCC(CCN)CC2)c2cn(CC3CCCCC3)c3ccccc23)c1 ; Cl] | 1-(4-(2- aminoethyl) piperidin-1-yl)-3-(1- (cyclohexylmethyl)- 1H-indol-3-yl)-3-(m- tolyl)propan-1-one hydrochloride salt | 522.16 | 485.34 | 486 | V |
| 182 | 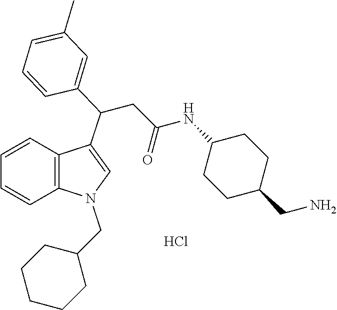 | N-((1r,4r)-4- (aminomethyl) cyclohexyl)- 3-(1-(cyclohexylmethyl)- 1H-indol-3-yl)-3-(m- tolyl)propanamide hydrochloride salt | 522.16 | 485.34 | 486 | V |
| 183 | 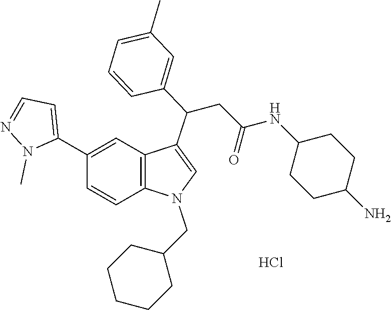 [SMILES: Cc1cccc(C(CC(=O)NC2CCC(N)CC2)c2cn(CC3CCCCC3)c3ccc(-c4ccnn4C)cc23)c1 ; Cl] | N-(4-aminocyclohexyl)- 3-(1-(cyclohexylmethyl)- 5-(1-methyl-1H- pyrazol-5-yl)- 1H-indol-3-yl)-3-(m- tolyl)propanamide hydrochloride salt | 588.23 | 551.36 | 552 | VI |
| 186 | 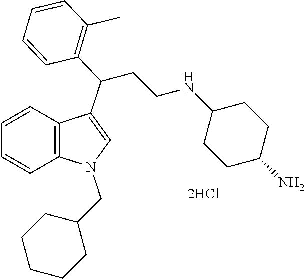 | N1-(3-(1- (cyclohexylmethyl)-1H- indol-3-yl)-3-(o- tolyl)propyl)cyclohexane- 1,4-diamine hydrochloride salt | 530.62 | 457.35 | 458 | II |
| 188 | 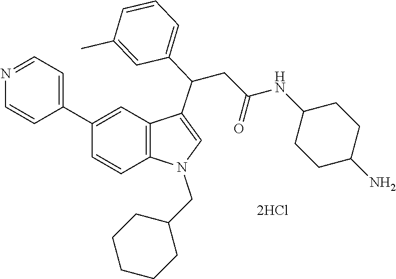 [SMILES: Cc1cccc(C(CC(=O)NC2CCC(N)CC2)c2cn(CC3CCCCC3)c3ccc(-c4ccncc4)cc23)c1 ; "2HCl"] | N-(4-aminocyclohexyl)-3- (1-(cyclohexylmethyl)-5- (pyridin-4-yl)-1H-indol-3- yl)-3-(m-tolyl) propanamide hydrochloride salt | 621.68 | 548.35 | 549 | VI |
| 189 | 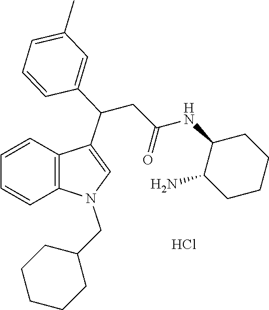 [SMILES: Cc1cccc(C(CC(=O)N[C@H]2CCCC[C@@H]2N)c2cn(CC3CCCCC3)c3ccccc23)c1 ; Cl] | N-((1S,2S)-2- aminocyclohexyl)-3-(1- (cyclohexylmethyl)-1H- indol-3-yl)-3-(m- tolyl)propanamide hydrochloride salt | 508.14 | 471.32 | 472 | V |
| 190 | 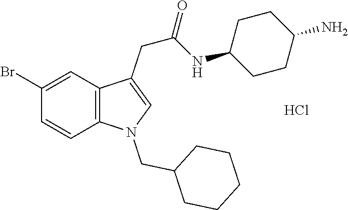 [SMILES: N[C@H]1CC[C@H](NC(=O)Cc2cn(CC3CCCCC3)c3ccc(Br)cc23)CC1 ; Cl] | N-((1R,4R)-4- aminocyclohexyl)-2-(5- bromo-1- (cyclohexylmethyl)-1H- indol-3-yl)acetamide hydrochloride salt | 482.88 | 445.17 | 446 | V |
| 191 | 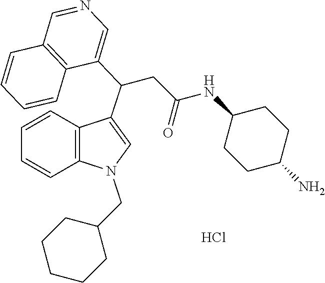 [SMILES: N[C@H]1CC[C@H](NC(=O)CC(c2cncc3ccccc23)c2cn(CC3CCCCC3)c3ccccc23)CC1 ; Cl] | N-((1R,4R)-4- aminocyclohexyl)-3-(1- (cyclohexylmethyl)-1H- indol-3-yl)-3- (isoquinolin-4- yl)propanamide hydrochloride salt | 567.64 | 508.32 | 495 | V |
| 197 | 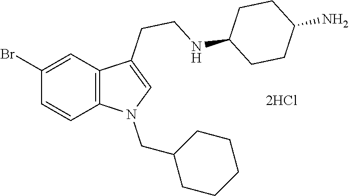 [SMILES: N[C@H]1CC[C@H](NCCc2cn(CC3CCCCC3)c3ccc(Br)cc23)CC1] | (1R,4R)-N1-(2- (5-bromo-1- (cyclohexylmethyl)-1H- indol-3-yl)ethyl) cyclohexane-1,4- diamine hydrochloride salt | 505.36 | 431.19 | 432 | VIII |
| 198 | 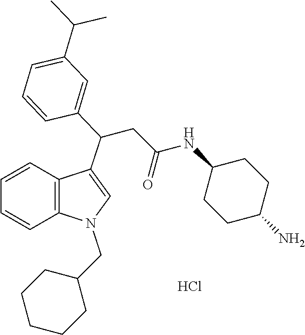 [SMILES: CC(C)c1cccc(C(CC(=O)N[C@H]2CC[C@H](N)CC2)c2cn(CC3CCCCC3)c3ccccc23)c1 ; Cl] | N-((1R,4R)-4- aminocyclohexyl)-3-(1- (cyclohexylmethyl)-1H- indol-3-yl)-3-(3- isopropylphenyl) propanamide hydrochloride salt | 536.19 | 499.36 | NA | V |
| 199 | 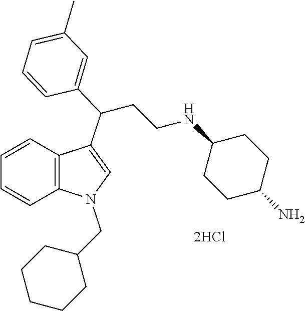 [SMILES: Cc1cccc(C(CCN[C@H]2CC[C@H](N)CC2)c2cn(CC3CCCCC3)c3ccccc23)c1 ; "2HCl"] | (1R,4R)-N1-(3-(1- (cyclohexylmethyl)-1H- indol-3-yl)-3-(m- tolyl)propyl) cyclohexane- 1,4-diamine hydrochloride | 530.62 | 457.35 | NA | II |
| 200 | 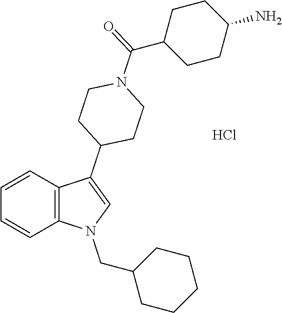 | (4-aminocyclohexyl) (4-(1-(cyclohexylmethyl)- 1H-indol-3-yl)piperidin-1- yl)methanone hydrochloride salt | 458.08 | 421.31 | NA | |
| 201 | 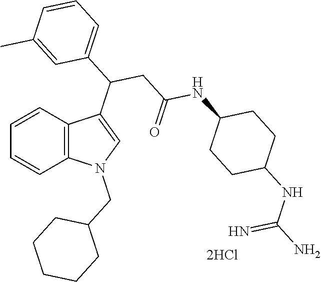 | 3-(1-(cyclohexylmethyl)- 1H-indol-3-yl)-N-(4- guanidinocyclohexyl)- 3-(m-tolyl) propanamide hydrochloride salt | 586.64 | 513.35 | 514 | V |
| 202 | 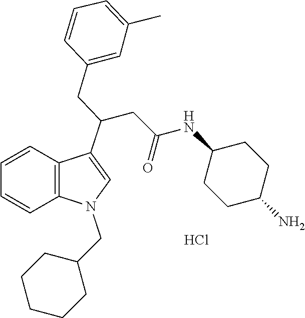 [SMILES: Cc1cccc(CC(CC(=O)N[C@H]2CC[C@H](N)CC2)c2cn(CC3CCCCC3)c3ccccc23)c1 ; Cl] | N-((1R,4R)-4- aminocyclohexyl)-3-(1- (cyclohexylmethyl)-1H- indol-3-yl)-4-(m- tolyl)butanamide hydrochloride salt | 522.16 | 485.34 | 486 | V |
| 203 | 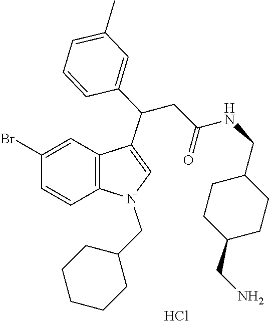 | N-((4- (aminomethyl)cyclohexyl) methyl)-3-(5-bromo-1- (cyclohexylmethyl)-1H- indol-3-yl)-3-(m- tolyl)propanamide hydrochloride salt | 615.09 | 577.27 | 580 | V |
| 204 | 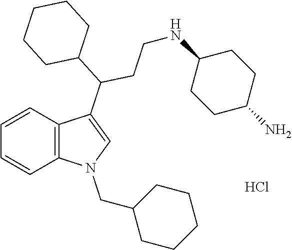 [SMILES: N[C@H]1CC[C@H](NCCC(c2cn(CC3CCCCC3)c3ccccc23)C2CCCCC2)CC1 ; Cl] | (1rR4R)-N1- (3-cyclohexyl- 3-(1-(cyclohexylmethyl)- 1H-indol-3-yl)propyl) cyclohexane-1,4- diamine hydrochloride salt | 522.64 | 449.38 | 450 | II |
| 205 | 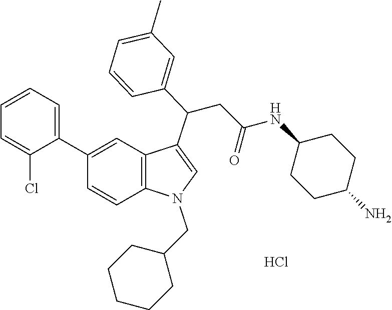 [SMILES: Cc1cccc(C(CC(=O)N[C@H]2CC[C@H](N)CC2)c2cn(CC3CCCCC3)c3ccc(-c4ccccc4Cl)cc23)c1 ; Cl] | N-((1R,4R)-4- aminocyclohexyl)- 3-(5-(2- chlorophenyl)-1- (cyclohexylmethyl)-1H- indol-3-yl)-3-(m- tolyl)propanamide hydrochloride salt | 618.68 | 581.32 | 582 | VI |
| 206 | 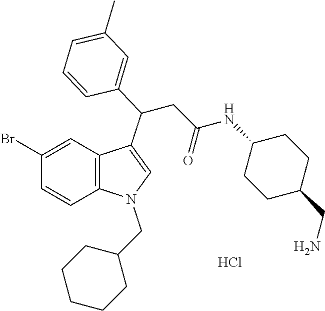 [SMILES: Cc1cccc(C(CC(=O)N[C@H]2CC[C@H](CN)CC2)c2cn(CC3CCCCC3)c3ccc(Br)cc23)c1 ; Cl] | N-((1R,4R)-4- (aminomethyl) cyclohexyl)- 3-(5-bromo-1- (cyclohexylmethyl)-1H- indol-3-yl)-3-(m- tolyl)propanamide hydrochloride salt | 601.06 | 563.25 | 486 | V |
| 207 | 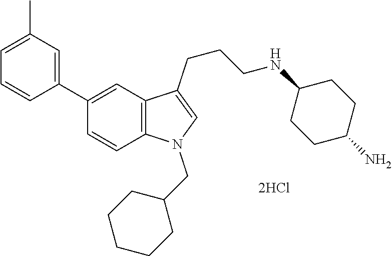 [SMILES: Cc1cccc(-c2ccc3c(c2)c(CCCN[C@H]2CC[C@H](N)CC2)cn3CC2CCCCC2)c1 ; "2HCl"] | (1R,4R)-N1-(3-(1- (cyclohexylmethyl)-5-(m- tolyl)-1H-indol-3-yl) propyl)cyclohexane-1,4- diamine hydrochloride salt | 530.62 | 457.35 | 458 | III |
| 208 | 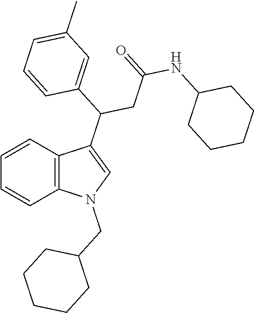 [SMILES: Cc1cccc(C(CC(=O)NC2CCCCC2)c2cn(CC3CCCCC3)c3ccccc23)c1] | N-cyclohexyl-3-(1- (cyclohexylmethyl)-1H- indol-3-yl)-3-(m- tolyl)propanamide | NA | 456.66 | 457 | V |
| 209 | 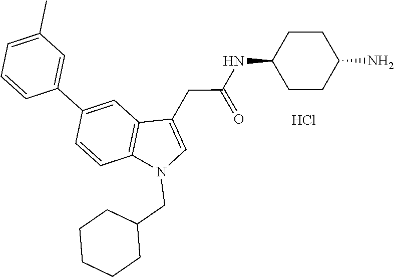 [SMILES: Cc1cccc(-c2ccc3c(c2)c(CC(=O)N[C@H]2CC[C@H](N)CC2)cn3CC2CCCCC2)c1 ; Cl] | N-((1R,4R)-4- aminocyclohexyl)-2-(1- (cyclohexylmethyl)-5- (m-tolyl)-1H-indol-3- yl)acetamide hydrochloride salt | 494.11 | 457.31 | 458 | VI |
| 210 | 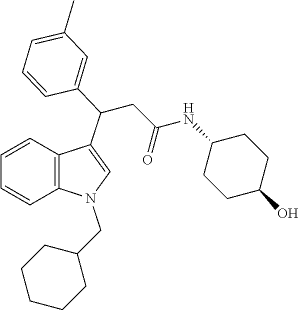 [SMILES: Cc1cccc(C(CC(=O)N[C@H]2CC[C@H](O)CC2)c2cn(CC3CCCCC3)c3ccccc23)c1] | 3-(1- (cyclohexylmethyl)- 1H-indol-3-yl)- N-((1R,4R)-4- hydroxycyclohexyl)-3- (m- tolyl)propanamide | NA | 472.66 | 473 | V |
| 211 | 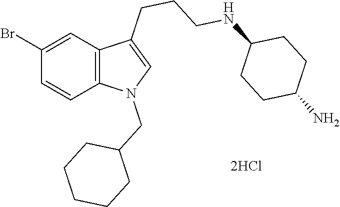 [SMILES: N[C@H]1CC[C@H](NCCCc2cn(CC3CCCCC3)c3ccc(Br)cc23)CC1 ; "2HCl"] | (1R,4R)-N1-(3- (5-bromo-1- (cyclohexylmethyl)- 1H- indol-3- yl)propyl)cyclohexane- 1,4-diamine hydrochloride salt | 519.39 | 445.21 | 446 | V |
| 213 | 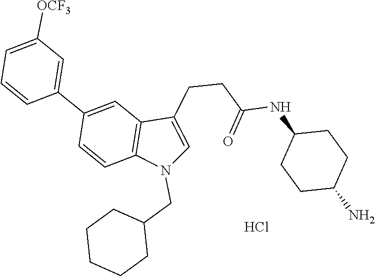 [SMILES: N[C@H]1CC[C@H](NC(=O)CCc2cn(CC3CCCCC3)c3ccc(-c4cccc(OC(F)(F)F)c4)cc23)CC1 ; Cl] | N-((1R,4R)-4- aminocyclohexyl)-3-(1- (cyclohexylmethyl)-5-(3- (trifluoromethoxy) phenyl)- 1H-indol-3-yl) propanamide hydrochloride salt | 578.11 | 541.29 | NA | VI |
| 214 | 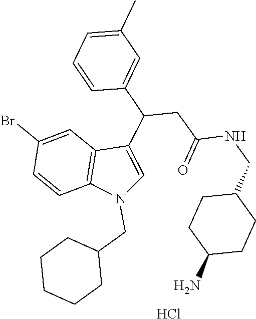 | N-(((1R,4R)-4- aminocyclohexyl) methyl)-3- (5-bromo-1- (cyclohexylmethyl)-1H- indol-3-yl)-3-(m- tolyl)propanamide hydrochloride salt | 601.06 | 563.25 | NA | V |
| 215 | 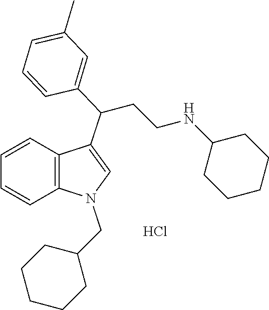 [SMILES: Cc1cccc(C(CCNC2CCCCC2)c2cn(CC3CCCCC3)c3ccccc23)c1 ; Cl] | N-(3-(1- (cyclohexylmethyl)- 1H-indol-3-yl)-3-(m- tolyl)propyl) cyclohexanamine hydrochloride salt | 479.14 | 442.33 | 443 | III |
| 216 | 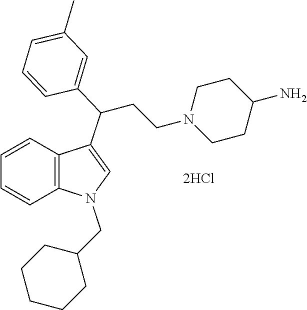 [SMILES: Cc1cccc(C(CCN2CCC(N)CC2)c2cn(CC3CCCCC3)c3ccccc23)c1 ; "2HCl"] | 1-(3-(1- (cyclohexylmethyl)- 1H-indol-3-yl)-3-(m- tolyl)propyl)piperidin-4- amine hydrochloride salt | 516.59 | 443.33 | 444 | II |
| 217 | 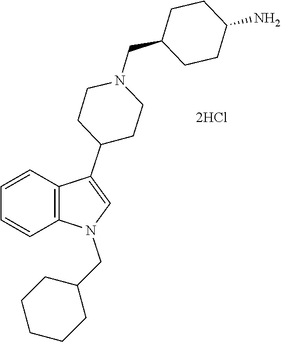 [SMILES: N[C@H]1CC[C@H](CN2CCC(c3cn(CC4CCCCC4)c4ccccc34)CC2)CC1 ; "2HCl"] | (1R,4R)-4-((4-(1- (cyclohexylmethyl)-1H- indol-3-yl)piperidin-1- yl)methyl)cyclohexan-1- amine hydrochloride salt | 480.56 | 407.33 | NA | |
| 218 | 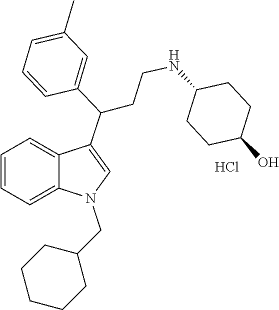 [SMILES: Cc1cccc(C(CCN[C@H]2CC[C@H](O)CC2)c2cn(CC3CCCCC3)c3ccccc23)c1 ; Cl] | (1R,4R)-4-((3-(1- (cyclohexylmethyl)- 1H-indol-3-yl)-3-(m- tolyl)propyl)amino) cyclohexan-1-ol hydrochloride salt | 495.14 | 458.33 | NA | II |
| 219 | 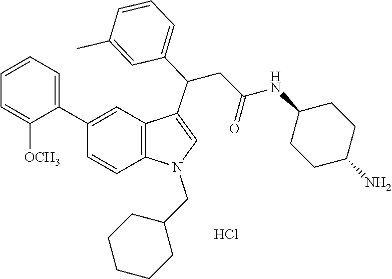 [SMILES: COc1ccccc1-c1ccc2c(c1)c(C(CC(=O)N[C@H]1CC[C@H](N)CC1)c1cccc(C)c1)cn2CC1CCCCC1 ; Cl] | N-((1S,4S)-4- aminocyclohexyl)-3-(1- (cyclohexylmethyl)-5- (2-(trifluoromethoxy) phenyl)-1H-indol- 3-yl)-3-(m- tolyl)propanamide hydrochloride salt | 614.26 | 577.37 | 578 | VI |
| 221 | 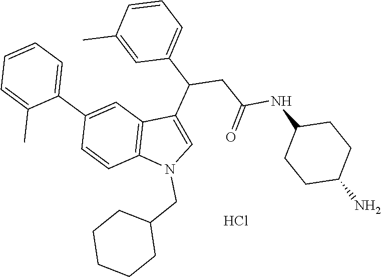 | N-((1S,4S)-4- aminocyclohexyl)-3-(1 - (cyclohexylmethyl)-5-(o- tolyl)-1H-indol-3-yl)-3- (m-tolyl)propanamide hydrochloride salt | 598.26 | 561.37 | 562 | VI |
| 222 | 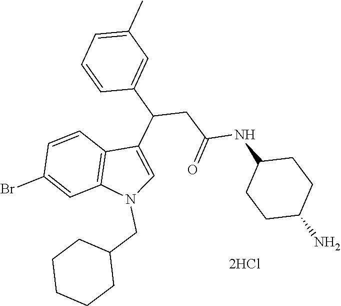 | N-((1S,4S)-4- aminocyclohexyl)-3-(6- bromo-1- (cyclohexylmethyl)-1H- indol-3-yl)-3-(m- tolyl)propanamide hydrochloride salt | 609.51 | 549.24 | 538 | V |
| 223 | 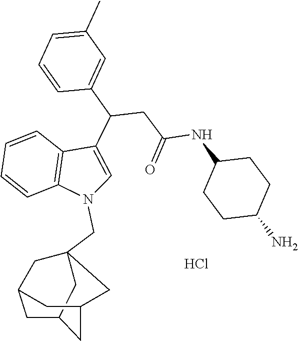 | 3-(1-(((1S,3S)- adamantan-1- yl)methyl)-1H- indol-3-yl)- N-((1s,4s)-4- aminocyclohexyl)-3- (m-tolyl)propanamide hydrochloride salt | 560.21 | 523.36 | 524 | V |
| 224 | 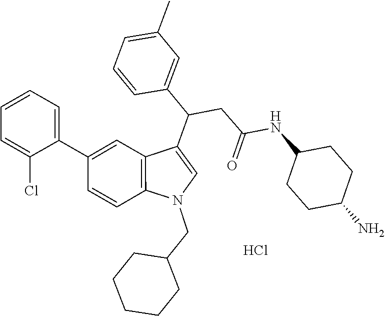 | N-((1S,4S)-4- aminocyclohexyl)- 3-(5-(2- chlorophenyl)-1- (cyclohexylmethyl)- 1H-indol-3-yl)-3-(m- tolyl)propanamide hydrochloride salt | 618 | 581.32 | NA | VI |
| 225 | 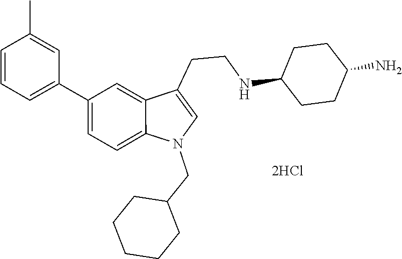 [SMILES: Cc1cccc(-c2ccc3c(c2)c(CCN[C@H]2CC[C@H](N)CC2)cn3CC2CCCCC2)c1] | (1R,4R)-N1-(2-(1- (cyclohexylmethyl)- 5-(m-tolyl)-1H-indol-3- yl)ethyl)cyclohexane- 1,4-diamine hydrochloride salt | 516.59 | 443.33 | NA | VI |
| 226 | 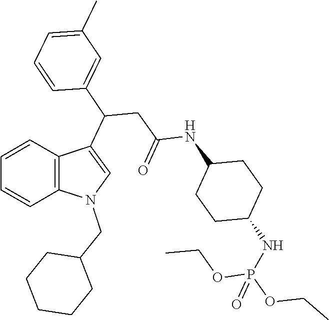 | diethyl ((1s,4s)-4-(3-(1- (cyclohexylmethyl)-1H- indol-3-yl)-3-(m- tolyl)propanamido) cyclohexyl) phosphoramidate | NA | 607.76 | 608 | V |
| 227 | 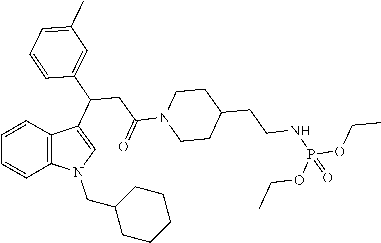 [SMILES: CCOP(=O)(NCCC1CCN(C(=O)CC(c2cccc(C)c2)c2cn(CC3CCCCC3)c3ccccc23)CC1)OCC] | diethyl (2-(1-(3-(1- (cyclohexylmethyl)-1H- indol-3-yl)-3-(m- tolyl)propanoyl) piperidin-4- yl)ethyl)phosphoramidate | NA | 621.79 | 622 | V |
| 228 | 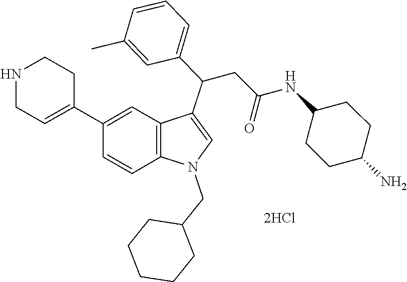 | N-((1S,4S)-4- aminocyclohexyl)-3-(1- (cyclohexylmethyl)-5-(1, 2,3,6-tetrahydropyridin- 4-yl)-1H-indol-3-yl)-3- (m-tolyl)propanamide hydrochloride salt | 625.71 | 552.38 | 553 | VI |
| 229 | 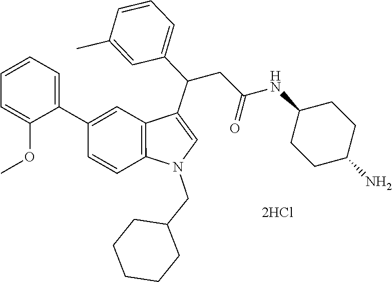 [SMILES: COc1ccccc1-c1ccc2c(c1)c(C(CC(=O)N[C@H]1CC[C@H](N)CC1)c1cccc(C)c1)cn2CC1CCCCC1 ; "2HCl"] | (1R,4R)-N1-(3-(1- (cyclohexylmethyl)-5- (2-methoxyphenyl)-1H- indol-3-yl)-3-(m- tolyl)propyl)cyclohexane- 1,4-diamine hydrochloride salt | 636.74 | 563.39 | 564 | VI |
| 230 | 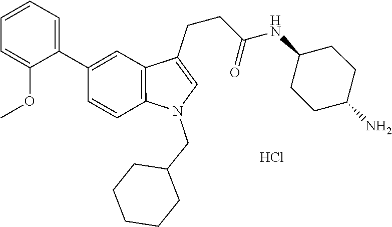 [SMILES: COc1ccccc1-c1ccc2c(c1)c(CCC(=O)N[C@H]1CC[C@H](N)CC1)cn2CC1CCCCC1 ; Cl] | N-((1R,4R)-4- aminocyclohexyl)-3-(1- (cyclohexylmethyl)-5-(2- methoxyphenyl)-1H- indol-3-yl)propanamide hydrochloride salt | 524.14 | 487.32 | 488 | VI |
| 231 | 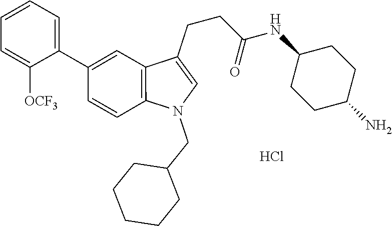 [SMILES: N[C@H]1CC[C@H](NC(=O)CCc2cn(CC3CCCCC3)c3ccc(-c4ccccc4OC(F)(F)F)cc23)CC1 ; Cl] | N-((1R,4R)-4- aminocyclohexyl)-3-(1- (cyclohexylmethyl)-5-(2- (trifluoromethoxy)phenyl)- 1H-indol-3-yl) propanamide hydrochloride salt | 578.11 | 541.29 | 542 | VI |
| 232 | 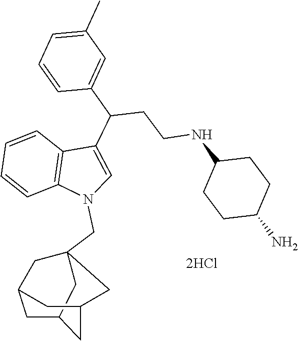 | (1S,4S)-N1-(3- (1-(((1S,4S)- adamantan-1-yl)methyl)- 1H-indol-3-yl)-3-(m- tolyl)propyl)cyclohexane- 1,4-diamine hydrochloride salt | 582.69 | 509.38 | 510 | II |
| 234 | 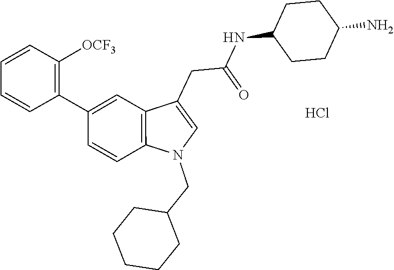 | N-((1S,4S)-4- aminocyclohexyl)-2-(1- (cyclohexylmethyl)-5- (2-(trifluoromethoxy) phenyl)-1H-indol-3- yl)acetamide hydrochloride salt | 564.08 | 527.38 | 528 | VI |
| 235 | 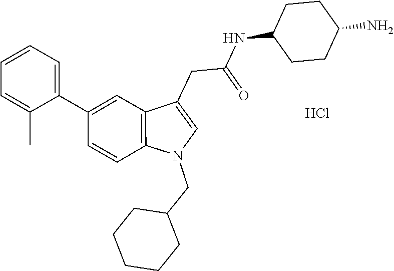 | N-((1S,4S)-4- aminocyclohexyl)-2-(1- (cyclohexylmethyl)-5- (o-tolyl)-1H-indol-3- yl)acetamide hydrochloride salt | 494.11 | 457.31 | 458 | VI |
| 236 | 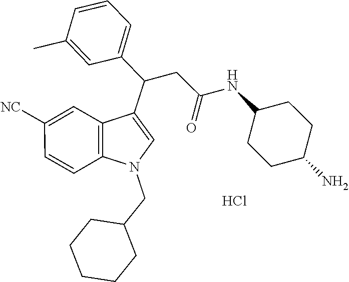 | N-((1S,4S)-4- aminocyclohexyl)-3-(5- cyano-1- (cyclohexylmethyl)-1H- indol-3-yl)-3-(m- tolyl)propanamide hydrochloride salt | 533.15 | 496.32 | 497 | V |
| 237 | 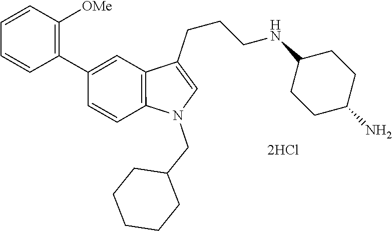 | (1S,4S)-N1-(3-(1- (cyclohexylmethyl)-5- (2-methoxyphenyl)- 1H-indol-3- yl)propyl)cyclohexane- 1,4-diamine hydrochloride salt | 546.61 | 473.34 | 488 | III |
| 238 | 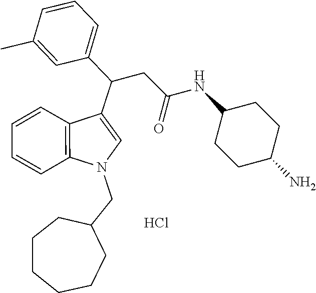 | N-((1S,4S)-4- aminocyclohexyl)-3-(1- (cycloheptylmethyl)- 1H-indol-3-yl)-3-(m- tolyl)propanamide hydrochloride salt | 522.16 | 485.34 | 486 | V |
| 239 | 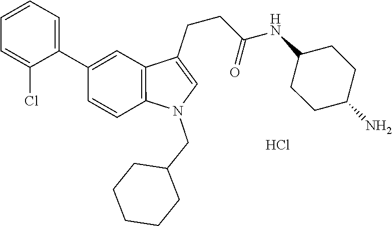 | N-((1S,4S)-4- aminocyclohexyl)-3-(5- (2-chlorophenyl)-1- (cyclohexylmethyl)-1H- indol-3-yl)propanamide hydrochloride salt | 528.56 | 491.27 | NA | VI |
| 240 | 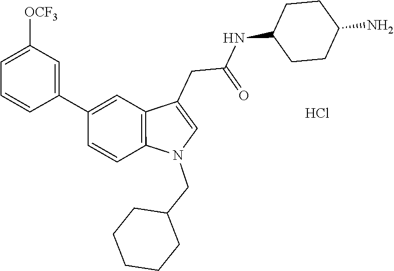 | N-((1S,4S)-4- aminocyclohexyl)-2-(1- (cyclohexylmethyl)-5- (3-(trifluoromethoxy) phenyl)-1H-indol- 3-yl)acetamide hydrochloride salt | 564.08 | 527.24 | 528 | VI |
| 241 | 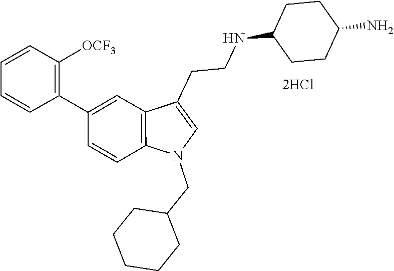 | (1S,4S)-N1-(2-(1- (cyclohexylmethyl)-5- (2-(trifluoromethoxy) phenyl)-1H-indol-3- yl)ethyl)cyclohexane-1,4- diamine hydrochloride salt | 586.56 | 513.30 | 514 | III |
| 242 | 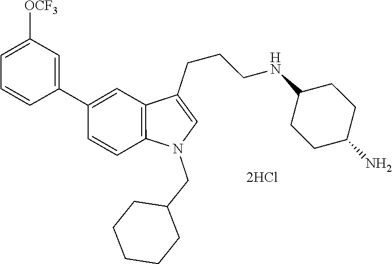 | (1S,4S)-N1-(3-(1- (cyclohexylmethyl)-5-(3- (trifluoromethoxy) phenyl)-1H-indol-3- yl)propyl)cyclohexane- 1,4-diamine hydrochloride salt | 600.59 | 527.31 | 528 | III |
| 243 | 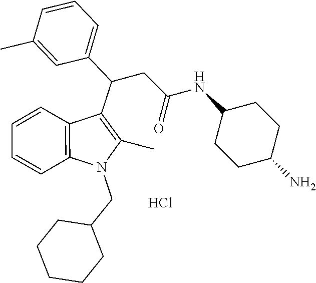 | N-((1S,4S)-4- aminocyclohexyl)-3-(1- (cyclohexylmethyl)-2- methyl-1H-indol-3-yl)-3- (m-tolyl)propanamide hydrochloride salt | 522.16 | 485.34 | NA | V |
| 244 | 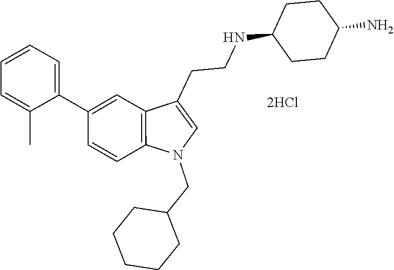 [SMILES: Cc1ccccc1-c1ccc2c(c1)c(CCN[C@H]1CC[C@H](N)CC1)cn2CC1CCCCC1 ; "2HCl"] | (1R,4R)-N1-(2-(1- (cyclohexylmethyl)-5-(o- tolyl)-1H-indol-3- yl)ethyl)cyclohexane-1,4- diamine hydrochloride salt | 516.59 | 443.33 | 444 | III |
| 245 | 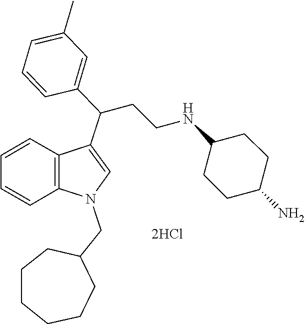 | (1S,4S)-N1-(3-(1- (cycloheptylmethyl)-1H- indol-3-yl)-3-(m- tolyl)propyl)cyclohexane- 1,4-diamine hydrochloride salt | 544.64 | 471.36 | 472 | II |
| 247 | 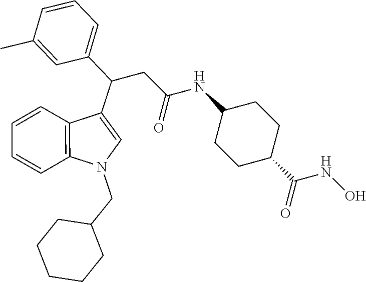 | (1S,4S)-4-(3-(1- (cyclohexylmethyl)-1H- indol-3-yl)-3-(m- tolyl)propanamido)-N- hydroxycyclohexane-1- carboxamide hydrochloride salt | NA | 515.69 | 516 | V |
| 255 | 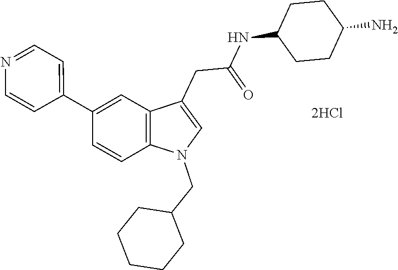 | N-((1S,4S)-4- aminocyclohexyl)-2-(1- (cyclohexylmethyl)-5- (pyridin-4-yl)-1H-indol- 3-yl)acetamide hydrochloride salt | 517.53 | 444.29 | 445 | VI |
| 259 | 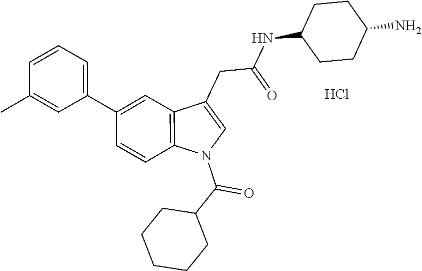 | N-((1S,4S)-4- aminocyclohexyl)-2-(1- (cyclohexanecarbonyl)- 5-(m-tolyl)-1H-indol-3- yl)acetamide hydrochloride salt | 508.12 | 471.29 | 486 | VI |
| 264 | 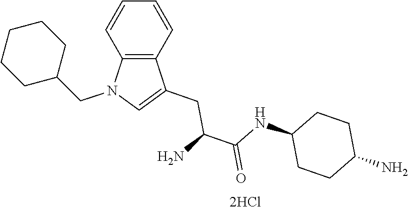 [SMILES: N[C@@H](Cc1cn(CC2CCCCC2)c2ccccc12)C(=O)N[C@H]1CC[C@H](N)CC1] | (S)-2-amino-N-((1R,4S)- 4-aminocyclohexyl)-3-(1- (cyclohexylmethyl)-1H- indol-3-yl)propanamide hydrochloride salt | 469.49 | 396.27 | NA | |
| 265 | 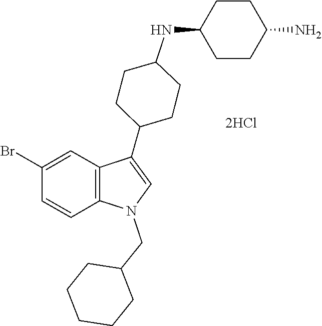 [SMILES: N[C@H]1CC[C@H](NC2CCC(c3cn(CC4CCCCC4)c4ccc(Br)cc34)CC2)CC1 ; "2HCl"] | (1r,4r)-N1-(4-(5-bromo-1- (cyclohexylmethyl)-1H- indol-3-yl) cyclohexyl)cyclohexane- 1,4-diamine hydrochloride salt | 559.45 | 485.24 | NA | |
| 266 | 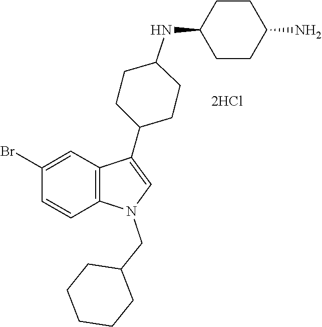 [SMILES: N[C@H]1CC[C@H](NC2CCC(c3cn(CC4CCCCC4)c4ccc(Br)cc34)CC2)CC1 ; "2HCl"] | (1r,4r)-N1-(4-(5-bromo-1- (cyclohexylmethyl)-1H- indol-3-yl) cyclohexyl)cyclohexane- 1,4-diamine hydrochloride salt | 559.45 | 485.24 | NA | |
| 268 | 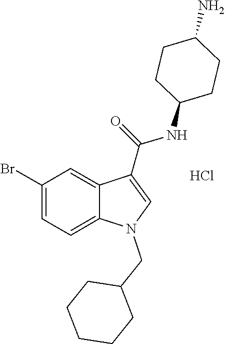 [SMILES: N[C@H]1CC[C@H](NC(=O)c2cn(CC3CCCCC3)c3ccc(Br)cc23)CC1 ; Cl] | N-((1R,4R)-4- aminocyclohexyl)- 5-bromo- 1-(cyclohexylmethyl)- 1H-indole-3- carboxamide hydrochloride salt | 468.86 | 431.26 | NA | |
| 269 | 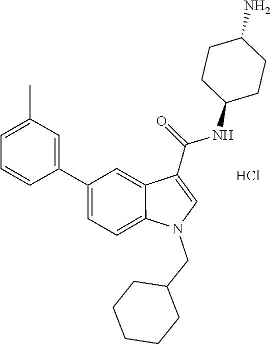 [SMILES: Cc1cccc(-c2ccc3c(c2)c(C(=O)N[C@H]2CC[C@H](N)CC2)cn3CC2CCCCC2)c1 ; Cl] | N-((1r,4r)-4- aminocyclohexyl)-1- (cyclohexylmethyl)-5- (m-tolyl)-1H-indole-3- carboxamide hydrochloride salt | 480.08 | 443.29 | NA | |
| 270 | 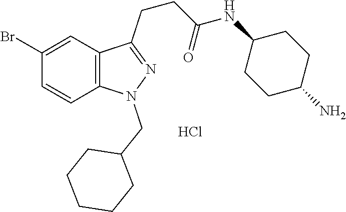 [SMILES: N[C@H]1CC[C@H](NC(=O)CCc2nn(CC3CCCCC3)c3ccc(Br)cc23)CC1 ; Cl] | N-((1r,4r)-4- aminocyclohexyl)-3-(5- bromo-1- (cyclohexylmethyl)-1H- indazol-3-yl)propanamide hydrochloride salt | 497.9 | 46018 | 462 | |
| 272 | 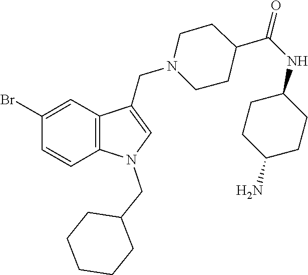 [SMILES: N[C@H]1CC[C@H](NC(=O)C2CCN(Cc3cn(CC4CCCCC4)c4ccc(Br)cc34)CC2)CC1] | N-((1R,4R)-4- aminocyclohexyl)-1-((5- bromo-1- (cyclohexylmethyl)-1H- indol-3- yl)methyl)piperidine-4- carboxamide | NA | 528.56 | NA | |
| 274 | 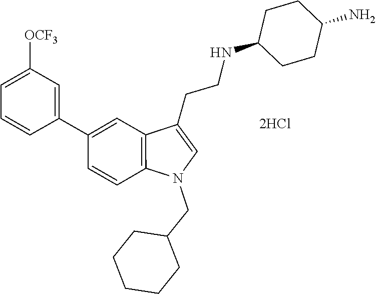 [SMILES: N[C@H]1CC[C@H](NCCc2cn(CC3CCCCC3)c3ccc(-c4cccc(OC(F)(F)F)c4)cc23)CC1 ; "2HCl"] | (3-(2-(((1S,4S)-4- aminocyclohexyl)amino) ethyl)-5-(3- (trifluoromethoxy)phenyl)- 1H-indol-1- yl)(cyclohexyl)methanone hydrochloride salt | 586.56 | 513.30 | 514 | II |
| 275 | 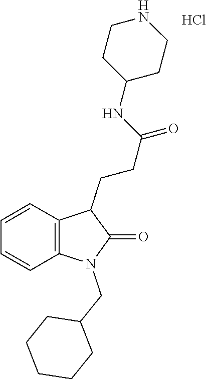 [SMILES: O=C(CCC1C(=O)N(CC2CCCCC2)c2ccccc21)NC1CCNCC1 ; Cl] | 3-(1-(cyclohexylmethyl)- 2-oxoindolin-3-yl)-N- (piperidin-4-yl) propanamide hydrochloride salt | 419.99 | 383.36 | NA | |
| 276 | 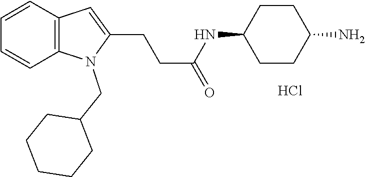 [SMILES: N[C@H]1CC[C@H](NC(=O)CCc2cc3ccccc3n2CC2CCCCC2)CC1 ; Cl] | N-((1R,4R)-4- aminocyclohexyl)-3-(1- (cyclohexylmethyl)-1H- indol-2-yl)propanamide hydrochloride salt | 418.02 | 381.28 | NA | |
| 277 | 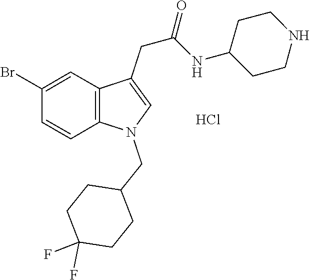 [SMILES: O=C(Cc1cn(CC2CCC(F)(F)CC2)c2ccc(Br)cc12)NC1CCNCC1 ; Cl] | 2-(5-bromo-1-((4,4- difluorocyclohexyl) methyl)-1H-indol- 3-yl)-N-(piperidin- 4-yl)acetamide hydrochloride salt | 504.84 | 467.18 | NA | IX |
| 278 | 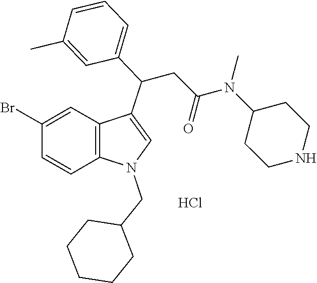 [SMILES: Cc1cccc(C(CC(=O)N(C)C2CCNCC2)c2cn(CC3CCCCC3)c3ccc(Br)cc23)c1 ; Cl] | 3-(5-bromo-1- (cyclohexylmethyl)-1H- indol-3-yl)-N-methyl-N- (piperidin-4-yl)-3-(m- tolyl)propanamide hydrochloride salt | 587.03 | 549.24 | 552 | V |
| 279 | 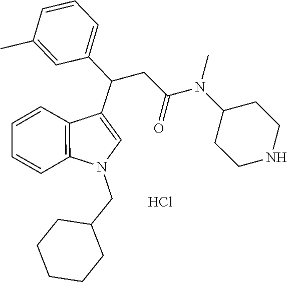 [SMILES: Cc1cccc(C(CC(=O)N(C)C2CCNCC2)c2cn(CC3CCCCC3)c3ccccc23)c1 ; Cl] | 3-(1-(cyclohexylmethyl)- 1H-indol-3-yl)- N-methyl-N- (piperidin-4-yl)-3-(m- tolyl)propanamide hydrochloride salt | 508.14 | 471.32 | 472 | V |
| 280 | 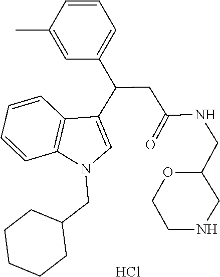 [SMILES: Cc1cccc(C(CC(=O)NCC2CNCCO2)c2cn(CC3CCCCC3)c3ccccc23)c1 ; Cl] | 3-(1-(cyclohexylmethyl)- 1H-indol-3-yl)-N- (morpholin-2- ylmethyl)-3- (m-tolyl)propanamide hydrochloride salt | 510.11 | 473.30 | 474 | V |
| 281 | 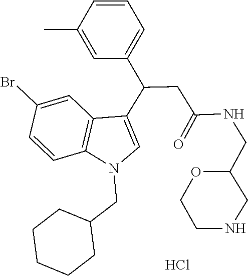 [SMILES: Cc1cccc(C(CC(=O)NCC2CNCCO2)c2cn(CC3CCCCC3)c3ccc(Br)cc23)c1 ; Cl] | 3-(5-bromo-1- (cyclohexylmethyl)-1H- indol-3-yl)-N- (morpholin-2- ylmethyl)-3-(m- tolyl)propanamide hydrochloride salt | 589.01 | 551.21 | 552 | V |
| 282 | 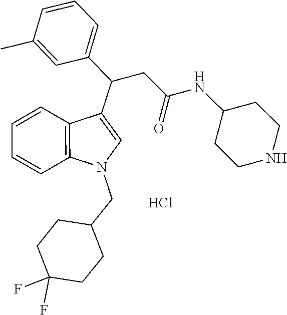 [SMILES: Cc1cccc(C(CC(=O)NC2CCNCC2)c2cn(CC3CCC(F)(F)CC3)c3ccccc23)c1 ; Cl] | 3-(1-((4,4- difluorocyclohexyl) methyl)-1H-indol-3- yl)-N-(piperidin- 4-yl)-3-(m- tolyl)propanamide hydrochloride salt | 530.09 | 493.29 | 494 | V |
| 284 | 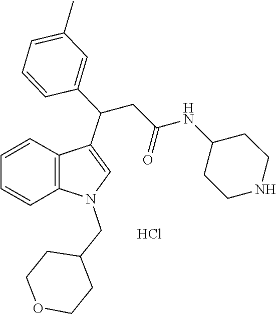 [SMILES: Cc1cccc(C(CC(=O)NC2CCNCC2)c2cn(CC3CCOCC3)c3ccccc23)c1 ; Cl] | N-(piperidin-4-yl)-3-(1- ((tetrahydro-2H-pyran-4- yl)methyl)-1H- indol-3-yl)- 3-(m-tolyl)propanamide hydrochloride salt | 496.08 | 459.29 | 460 | V |
| 285 | 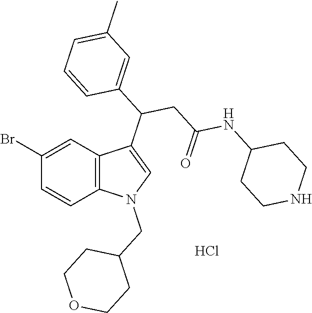 [SMILES: Cc1cccc(C(CC(=O)NC2CCNCC2)c2cn(CC3CCOCC3)c3ccc(Br)cc23)c1 ; Cl] | 3-(5-bromo-1- ((tetrahydro- 2H-pyran-4-yl) methyl)-1H- indol-3-yl)-N- (piperidin-4- yl)-3-(m-tolyl) propanamide hydrochloride salt | 574.98 | 537.20 | 538 | V |
| 286 | 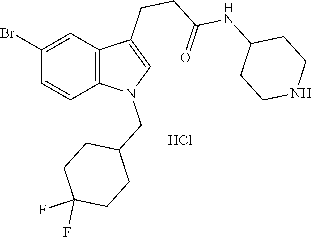 [SMILES: O=C(CCc1cn(CC2CCC(F)(F)CC2)c2ccc(Br)cc12)NC1CCNCC1 ; Cl] | 3-(5-bromo-1-((4,4- difluorocyclohexyl) methyl)-1H-indol- 3-yl)-N-(piperidin- 4-yl)propanamide hydrochloride salt | 518.87 | 481.15 | 482 | V |
| 287 | 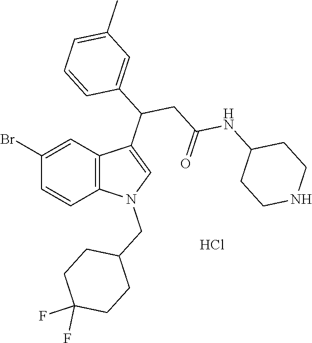 [SMILES: Cc1cccc(C(CC(=O)NC2CCNCC2)c2cn(CC3CCC(F)(F)CC3)c3ccc(Br)cc23)c1 ; Cl] | 3-(5-bromo-1-((4,4- difluorocyclohexyl) methyl)-1H-indol-3- yl)-N-(piperidin- 4-yl)-3-(m- tolyl)propanamide hydrochloride salt | 608.99 | 571.20 | 574 | V |
| 288 | 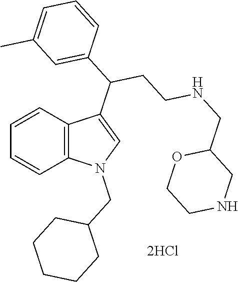 [SMILES: Cc1cccc(C(CCNCC2CNCCO2)c2cn(CC3CCCCC3)c3ccccc23)c1 ; "2HCl"] | 3-(1-(cyclohexylmethyl)- 1H-indol-3-yl)-N- (morpholin-2- ylmethyl)-3- (m-tolyl)propan-1-amine hydrochloride salt | 532.59 | 459.32 | 460 | II |
| 289 | 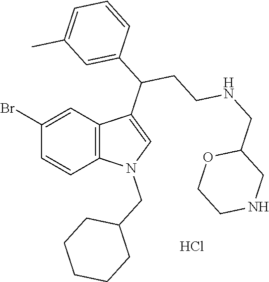 [SMILES: Cc1cccc(C(CCNCC2CNCCO2)c2cn(CC3CCCCC3)c3ccc(Br)cc23)c1 ; Cl] | 3-(5-bromo-1- (cyclohexylmethyl)-1H- indol-3-yl)-N- (morpholin-2- ylmethyl)-3-(m- tolyl)propan-1-amine hydrochloride salt | 611.48 | 537.28 | 538 | II |
| 290 | 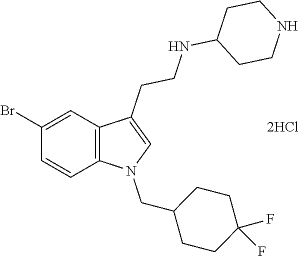 [SMILES: FC1(F)CCC(Cn2cc(CCNC3CCNCC3)c3cc(Br)ccc32)CC1 ; "2HCl"] | N-(2-(5-bromo-1-((4,4- difluorocyclohexyl) methyl)-1H-indol-3- yl)ethyl)piperidin-4-amine hydrochloride salt | 527.32 | 453.16 | 456 | II |
| 291 | 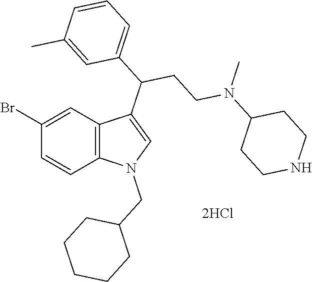 [SMILES: Cc1cccc(C(CCN(C)C2CCNCC2)c2cn(CC3CCCCC3)c3ccc(Br)cc23)c1 ; "2HCl"] | N-(3-(5-bromo-1- (cyclohexylmethyl)-1H- indol-3-yl)-3-(m- tolyl)propyl)-N- methylpiperidin-4-amine hydrochloride salt | 609.51 | 535.26 | | II |
| 292 | 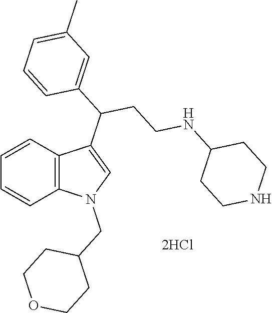 [SMILES: Cc1cccc(C(CCNC2CCNCC2)c2cn(CC3CCOCC3)c3ccccc23)c1 ; "2HCl"] | N-(3-(1-((tetrahydro-2H- pyran-4-yl)methyl)-1H- indol-3-yl)-3-(m- tolyl)propyl)piperidin-4- amine hydrochloride salt | 518.56 | 445.31 | 458 | II |
| 293 | 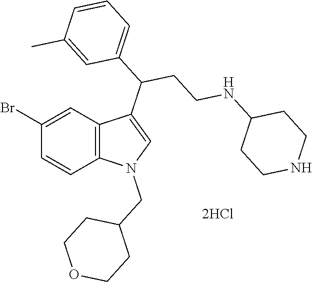 [SMILES: Cc1cccc(C(CCNC2CCNCC2)c2cn(CC3CCOCC3)c3ccc(Br)cc23)c1 ; "2HCl"] | N-(3-(5-bromo-1- ((tetrahydro-2H-pyran-4- yl)methyl)-1H-indol-3-yl)- 3-(m-tolyl)propyl) piperidin-4-amine hydrochloride salt | 597.46 | 523.22 | 526 | II |
| 295 | 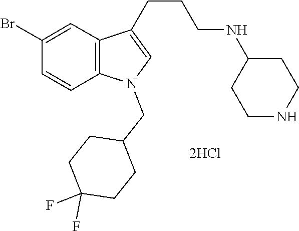 [SMILES: FC1(F)CCC(Cn2cc(CCCNC3CCNCC3)c3cc(Br)ccc32)CC1] | N-(3-(5-bromo-1-((4,4- difluorocyclohexyl) methyl)-1H-indol-3- yl)propyl)piperidin- 4-amine hydrochloride salt | 541.34 | 467.17 | 468 | II |
| 296 | 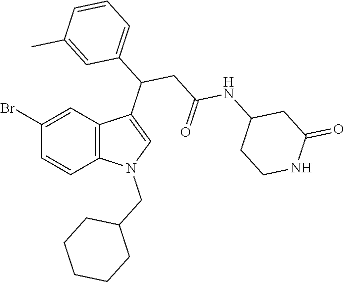 [SMILES: Cc1cccc(C(CC(=O)NC2CCNC(=O)C2)c2cn(CC3CCCCC3)c3ccc(Br)cc23)c1] | 3-(5-bromo-1- (cyclohexylmethyl)-1H- indol-3-yl)-N-(2- oxopiperidin-4-yl)-3-(m- tolyl)propanamide hydrochloride salt | 550.53 | 549.20 | 550 | V |
| 297 | 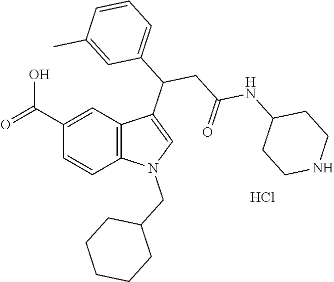 [SMILES: Cc1cccc(C(CC(=O)NC2CCNCC2)c2cn(CC3CCCCC3)c3ccc(C(=O)O)cc23)c1 ; Cl] | 1-(cyclohexylmethyl)-3-(3- oxo-3-(piperidin-4- ylamino)-1-(m- tolyl)propyl)-1H-indole-5- carboxylic acid hydrochloride salt | 538.12 | 501.30 | 502 | V |
| 298 | 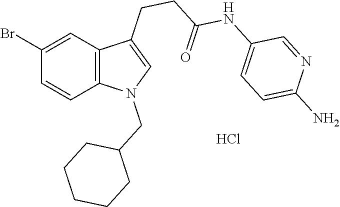 [SMILES: Nc1ccc(NC(=O)CCc2cn(CC3CCCCC3)c3ccc(Br)cc23)cn1 ; Cl] | N-(6-aminopyridin-3-yl)-3- (5-bromo-1- (cyclohexylmethyl)-1H- indol-3-yl)propanamide hydrochloride salt | 455.39 | 454.14 | 455 | V |
| 299 | 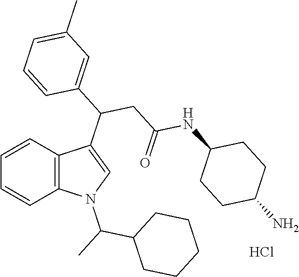 [SMILES: Cc1cccc(C(CC(=O)N[C@H]2CC[C@H](N)CC2)c2cn(C(C)C3CCCCC3)c3ccccc23)c1 ; Cl] | N-((1r,4r)-4- aminocyclohexyl)-3-(1-(1- cyclohexylethyl)-1H-indol- 3-yl)-3-(m- tolyl)propanamide hydrochloride salt | 522.16 | 485.34 | 486 | V |
| 300 | 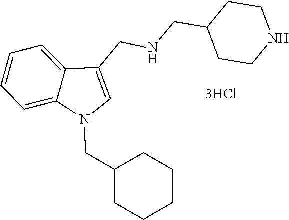 [SMILES: c1ccc2c(c1)c(CNCC1CCNCC1)cn2CC1CCCCC1] | 1-(1-(cyclohexylmethyl)- 1H-indol-3-yl)- N-(piperidin- 4-ylmethyl)methanamine hydrochloride salt | 448.9 | 339.27 | 340.4 | |
| 301 | 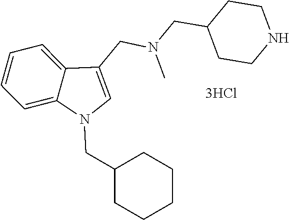 [SMILES: CN(Cc1cn(CC2CCCCC2)c2ccccc12)CC1CCNCC1] | 1-(1-(cyclohexylmethyl)- 1H-indol-3-yl)- N-methyl-N- (piperidin-4- ylmethyl)methanamine hydrochloride salt | 462.93 | 353.28 | 354.3 | |
| 302 | 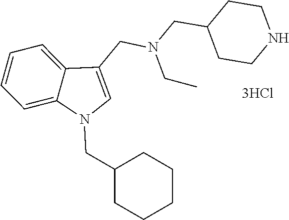 [SMILES: CCN(Cc1cn(CC2CCCCC2)c2ccccc12)CC1CCNCC1] | 1-(1-(cyclohexylmethyl)- 1H-indol-3-yl)- N-(piperidin- 4-ylmethyl)methanamine hydrochloride salt | 476.96 | 367.30 | 368.3 | |
| 303 | 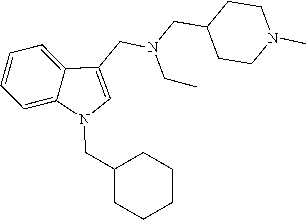 [SMILES: CCN(Cc1cn(CC2CCCCC2)c2ccccc12)CC1CCN(C)CC1] | N-((1-(cyclohexylmethyl)- 1H-indol-3-yl)methyl)-N- ((1-methylpiperidin-4- yl)methyl)ethanamine | NA | 381.61 | 382.3 | |
| 304 | 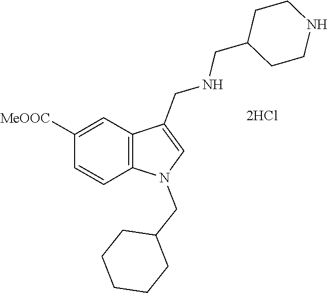 [SMILES: COC(=O)c1ccc2c(c1)c(CNCC1CCNCC1)cn2CC1CCCCC1] | methyl 1- (cyclohexylmethyl)-3- (((piperidin-4- ylmethyl)amino)methyl)- 1H-indole-5-carboxylate hydrochloride salt | 470.48 | 397.26 | 398.3 | |
| 305 | 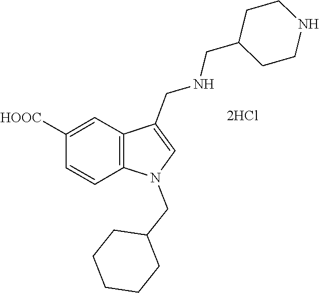 [SMILES: O=C(O)c1ccc2c(c1)c(CNCC1CCNCC1)cn2CC1CCCCC1] | 1-(cyclohexylmethyl)-3- (((piperidin-4- ylmethyl)amino)methyl)- 1H-indole-5- carboxylic acid hydrochloride salt | 456.45 | 383.26 | 384.3 | |
| 306 | 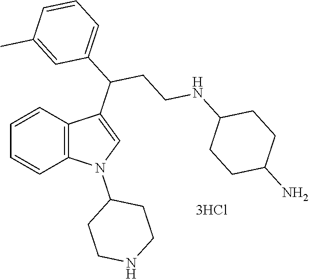 [SMILES: Cc1cccc(C(CCNC2CCC(N)CC2)c2cn(C3CCNCC3)c3ccccc23)c1 ; "3HCl"] | N1-(3-(1-(piperidin-4-yl)- 1H-indol-3-yl)-3-(m- tolyl)propyl)cyclohexane- 1,4-diamine hydrochloride salt | 554.04 | 444.33 | 445.3 | I |
| 307 | 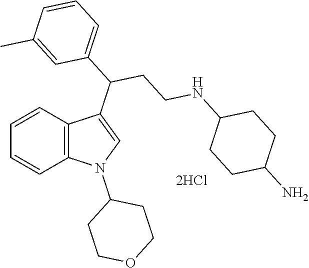 [SMILES: Cc1cccc(C(CCNC2CCC(N)CC2)c2cn(C3CCOCC3)c3ccccc23)c1 ; "2HCl"] | N1-(3-(1-(tetrahydro- 2H-pyran-4-yl)-1H- indol-3-yl)-3-(m- tolyl)propyl) cyclohexane- 1,4-diamine hydrochloride salt | 518.56 | 445.31 | 446.3 | I |
| 308 | 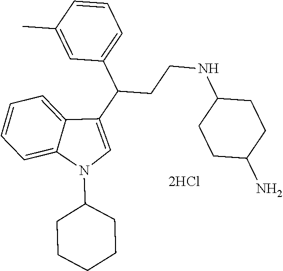 [SMILES: Cc1cccc(C(CCNC2CCC(N)CC2)c2cn(C3CCCCC3)c3ccccc23)c1 ; "2HCl"] | N1-(3-(1-cyclohexyl-1H- indol-3-yl)-3-(m- tolyl)propyl)cyclohexane- 1,4-diamine hydrochloride salt | 516.6 | 443.33 | 444.3 | I |
| 309 | 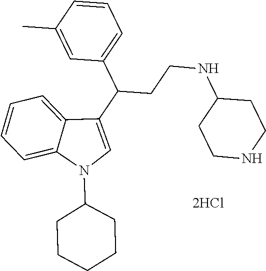 [SMILES: Cc1cccc(C(CCNC2CCNCC2)c2cn(C3CCCCC3)c3ccccc23)c1] | N-(3-(1-cyclohexyl-1H- indol-3-yl)-3-(m- tolyl)propyl)piperidin-4- amine hydrochloride salt | 502.57 | 429.31 | 430.3 | I |
| 310 | 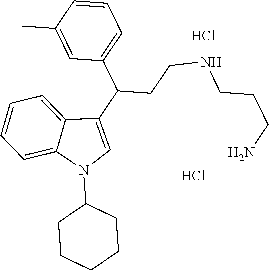 [SMILES: Cc1cccc(C(CCNCCCN)c2cn(C3CCCCC3)c3ccccc23)c1 ; Cl ; Cl] | N1-(3-(1-cyclohexyl-1H- indol-3-yl)-3-(m- tolyl)propyl)propane-1,3- diamine hydrochloride salt | 476.52 | 403.30 | 404.2 | I |
| 311 | 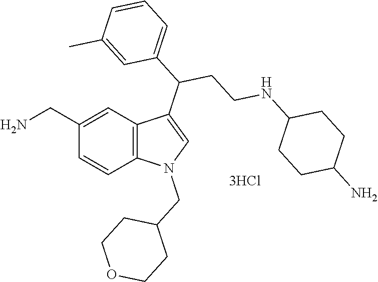 [SMILES: Cc1cccc(C(CCNC2CCC(N)CC2)c2cn(CC3CCOCC3)c3ccc(CN)cc23)c1 ; "3HCl"] | N1-(3-(5- (aminomethyl)-1- ((tetrahydro-2H- pyran-4- yl)methyl)-1H- indol-3-yl)-3-(m- tolyl)propyl) cyclohexane- 1,4-diamine hydrochloride salt | 598.09 | 488.35 | 489.3 | I |
| 312 | 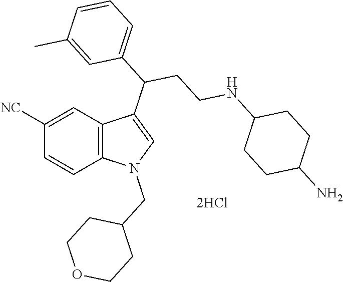 [SMILES: Cc1cccc(C(CCNC2CCC(N)CC2)c2cn(CC3CCOCC3)c3ccc(C#N)cc23)c1 ; "2HCl"] | 3-(3-((4- aminocyclohexyl) amino)-1-(m-tolyl) propyl)-1-((tetrahydro- 2H-pyran-4- yl)methyl)-1H-indole-5- carbonitrile hydrochloride salt | 557.6 | 484.32 | 485.2 | I |
| 313 | 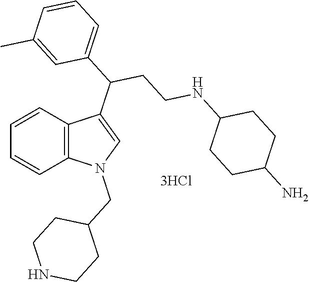 [SMILES: Cc1cccc(C(CCNC2CCC(N)CC2)c2cn(CC3CCNCC3)c3ccccc23)c1 ; "3HCl"] | N1-(3-(1-(piperidin-4- ylmethyl)-1H-indol- 3-yl)-3-(m-tolyl) propyl)cyclohexane- 1,4-diamine hydrochloride salt | 568.06 | 458.34 | 459.3 | I |
| 314 | 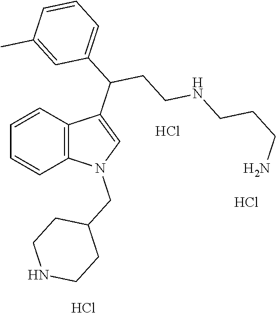 [SMILES: Cc1cccc(C(CCNCCCN)c2cn(CC3CCNCC3)c3ccccc23)c1 ; Cl ; Cl ; Cl] | N1-(3-(1-(piperidin-4- ylmethyl)-1H- indol-3-yl)-3- (m-tolyl)propyl)propane- 1,3-diamine hydrochloride salt | 528 | 418.31 | 419.3 | I |
| 315 | 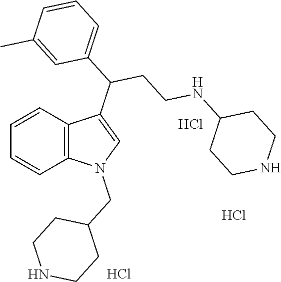 [SMILES: Cc1cccc(C(CCNC2CCNCC2)c2cn(CC3CCNCC3)c3ccccc23)c1 ; Cl ; Cl ; Cl] | N-(3-(1-(piperidin-4- ylmethyl)-1H- indol-3-yl)-3- (m-tolyl)propyl) piperichn-4- amine hydrochloride salt | 554.04 | 444.33 | 444.2 | I |
| 316 | 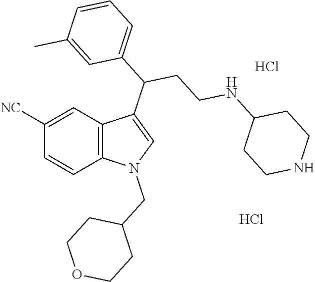 [SMILES: Cc1cccc(C(CCNC2CCNCC2)c2cn(CC3CCOCC3)c3ccc(C#N)cc23)c1 ; Cl ; Cl] | 3-(3-(piperidin- 4-ylamino)- 1-(m-tolyl)propyl)-1- ((tetrahydro-2H-pyran-4- yl)methyl)-1H-indole- 5-carbonitrile hydrochloride salt | 543.57 | 470.3 | 471.3 | I |
| 317 | 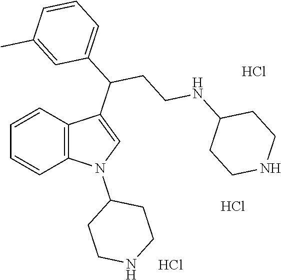 [SMILES: Cc1cccc(C(CCNC2CCNCC2)c2cn(C3CCNCC3)c3ccccc23)c1 ; Cl ; Cl ; Cl] | N-(3-(1- (piperidin-4-yl)-1H- indol-3-yl)-3-(m- tolyl)propyl)piperidin-4- amine hydrochloride salt | 540.01 | 430.31 | 431.2 | I |
| 318 | 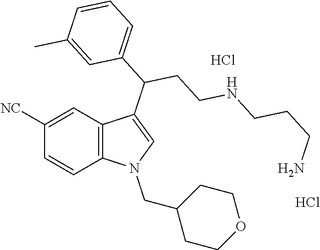 [SMILES: Cc1cccc(C(CCNCCCN)c2cn(CC3CCOCC3)c3ccc(C#N)cc23)c1 ; Cl ; Cl] | 3-(3-((3- aminopropyl) amino)-1-(m- tolyl)propyl)- 1-((tetrahydro- 2H-pyran-4- yl)methyl)-1H- indole-5-carbonitrile hydrochloride salt | 517.53 | 444.29 | 445.2 | I |
| 319 | 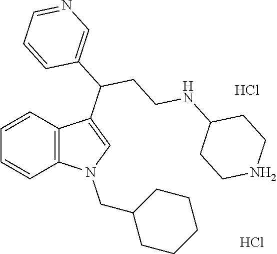 [SMILES: c1cncc(C(CCNC2CCNCC2)c2cn(CC3CCCCC3)c3ccccc23)c1 ; Cl ; Cl] | N1-(3-(1- (cyclohexylmethyl)-1H- indol-3-yl)-3-(pyridin- 3-yl)propyl) cyclohexane-1,4- diamine hydrochloride salt | 517.58 | 444.33 | 445.3 | I |
| 320 | 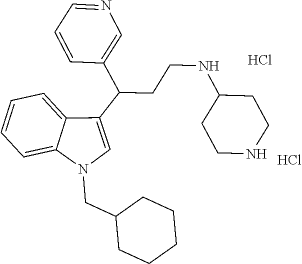 [SMILES: c1cncc(C(CCNC2CCNCC2)c2cn(CC3CCCCC3)c3ccccc23)c1 ; Cl ; Cl] | N-(3-(1- (cyclohexylmethyl)- 1H-indol-3-yl)-3- (pyridin-3- yl)propyl) piperidin-4-amine hydrochloride salt | 503.56 | 430.31 | 431.3 | I |
| 321 | 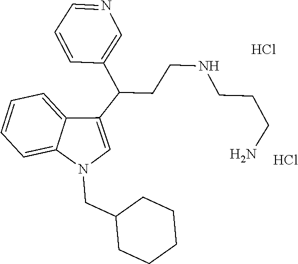 [SMILES: NCCCNCCC(c1cccnc1)c1cn(CC2CCCCC2)c2ccccc12 ; Cl ; Cl] | N1-(3-(1- (cyclohexylmethyl)- 1H-indol-3-yl)-3- (pyridin-3- yl)propyl)propane- 1,3-diamine hydrochloride salt | 477.52 | 404.29 | 405.3 | I |
| 322 | 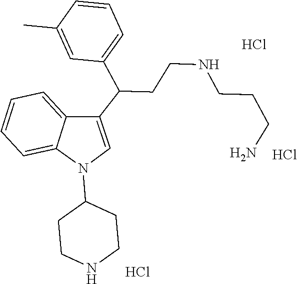 [SMILES: Cc1cccc(C(CCNCCCN)c2cn(C3CCNCC3)c3ccccc23)c1 ; Cl ; Cl ; Cl] | N1-(3-(1- (piperidin-4-yl)- 1H-indol-3-yl)-3-(m- tolyl)propyl)propane- 1,3-diamine hydrochloride salt | 513.98 | 404.29 | 405.3 | I |
| 323 | 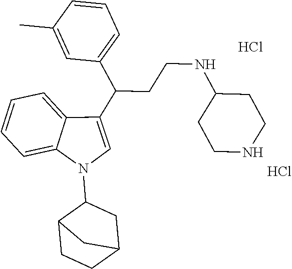 [SMILES: Cc1cccc(C(CCNC2CCNCC2)c2cn(C3CC4CCC3C4)c3ccccc23)c1 ; Cl ; Cl] | N1-(3-(1- (bicyclo[2.2.1] heptan-2-yl)- 1H-indol-3-yl)-3-(m- tolyl)propyl) piperidin-4-amine hydrochloride salt | 514.58 | 441.31 | 442.3 | I |
| 324 | 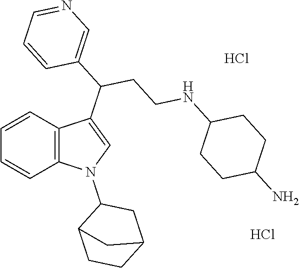 [SMILES: NC1CCC(NCCC(c2cccnc2)c2cn(C3CC4CCC3C4)c3ccccc23)CC1 ; Cl ; Cl] | N1-(3-(1- (bicyclo[2.2.1] heptan-2-yl)- 1H-indol-3-yl)- 3-(pyridin-3- yl)propyl)cyclohexane- 1,4-diamine hydrochloride salt | 515.57 | 442.31 | 443.3 | I |
| 325 | 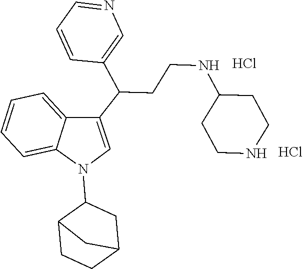 [SMILES: c1cncc(C(CCNC2CCNCC2)c2cn(C3CC4CCC3C4)c3ccccc23)c1 ; Cl ; Cl] | N1-(3-(1- (bicyclo[2.2.1] heptan-2-yl)- 1H-indol-3-yl)- 3-(pyridin-3- yl)propyl)piperidin- 4-amine hydrochloride salt | 501.54 | 428.29 | 429.3 | I |
| 326 | 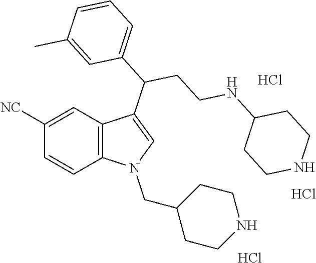 [SMILES: Cc1cccc(C(CCNC2CCNCC2)c2cn(CC3CCNCC3)c3ccc(C#N)cc23)c1 ; Cl ; Cl ; Cl] | 3-(3-(piperidin-4- ylamino)-1-(m- tolyl)propyl)-1- (piperidin-4-ylmethyl)- 1H-indole-5-carbonitrile hydrochloride salt | 579.05 | 469.32 | 470.3 | II |
| 327 | 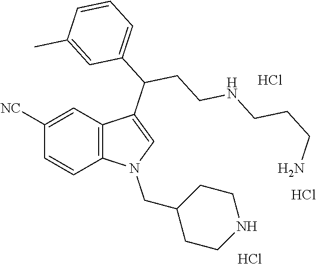 [SMILES: Cc1cccc(C(CCNCCCN)c2cn(CC3CCNCC3)c3ccc(C#N)cc23)c1 ; Cl ; Cl ; Cl] | 3-(3-((3- aminopropyl) amino)-1-(m- tolyl)propyl)-1- (piperidin-4- ylmethyl)-1H-indole-5- carbonitrile hydrochloride salt | 553.01 | 443.3 | 444.3 | II |
| 328 | 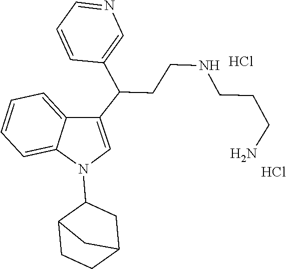 [SMILES: NCCCNCCC(c1cccnc1)c1cn(C2CC3CCC2C3)c2ccccc12 ; Cl ; Cl] | N1-(3-(1- (bicyclo[2.2.1] heptan-2-yl)- 1H-indol-3-yl)- 3-(pyridin-3- yl)propyl)propane-1,3- diamine hydrochloride salt | 475.5 | 402.28 | 403.3 | I |
| 329 | 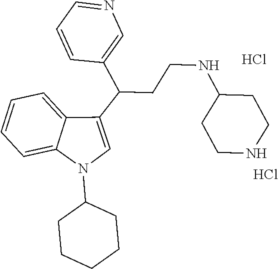 [SMILES: c1cncc(C(CCNC2CCNCC2)c2cn(C3CCCCC3)c3ccccc23)c1 ; Cl ; Cl] | N-(3-(1-cyclohexyl-1H- indol-3-yl)-3-(pyridin-3- yl)propyl)piperidin- 4-amine hydrochloride salt | 489.53 | 416.29 | 417.3 | I |
| 330 | 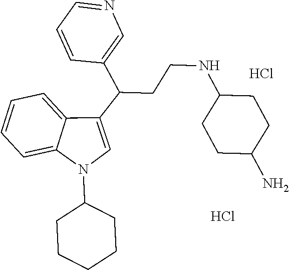 [SMILES: NC1CCC(NCCC(c2cccnc2)c2cn(C3CCCCC3)c3ccccc23)CC1 ; Cl ; Cl] | N1-(3-(1-cyclohexyl-1H- indol-3-yl)-3-(pyridin-3- yl)propyl)cyclohexane- 1,4-diamine hydrochloride salt | 503.56 | 430.31 | 431.3 | I |
| 331 | 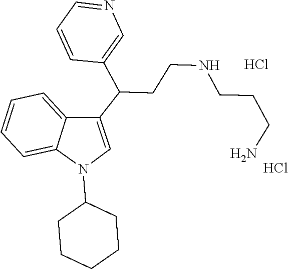 [SMILES: NCCCNCCC(c1cccnc1)c1cn(C2CCCCC2)c2ccccc12 ; Cl ; Cl] | N1-(3-(1-cyclohexyl-1H- indol-3-yl)-3-(pyridin-3- yl)propyl)propane-1,3- diamine hydrochloride salt | 463.49 | 390.28 | 391.3 | I |
| 332 | 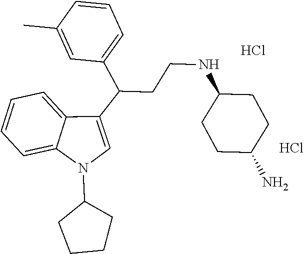 [SMILES: Cc1cccc(C(CCN[C@H]2CC[C@H](N)CC2)c2cn(C3CCCC3)c3ccccc23)c1 ; Cl ; Cl] | (1R,4R)-N1-(3-(1- cyclopentyl-1H- indol-3-yl)-3-(m- tolyl)propyl) cyclohexane- 1,4-diamine hydrochloride salt | 502.57 | 429.31 | 430.3 | I |
| 333 | 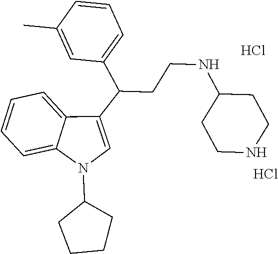 [SMILES: Cc1cccc(C(CCNC2CCNCC2)c2cn(C3CCCC3)c3ccccc23)c1 ; Cl ; Cl] | N-(3-(1-cyclopentyl-1H- indol-3-yl)-3-(m- tolyl)propyl)piperidin-4- amine hydrochloride salt | 488.54 | 415.30 | 416.3 | I |
| 334 | 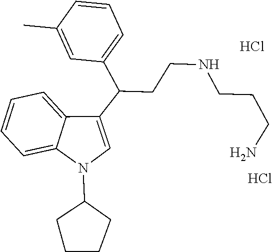 [SMILES: Cc1cccc(C(CCNCCCN)c2cn(C3CCCC3)c3ccccc23)c1 ; Cl ; Cl] | N1-(3-(1- cyclopentyl-1H- indol-3-yl)-3-(m- tolyl)propyl)propane- 1,3-diamine hydrochloride salt | 462.5 | 389.28 | 390.3 | I |
| 335 | 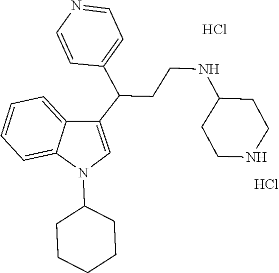 [SMILES: c1ccc2c(c1)c(C(CCNC1CCNCC1)c1ccncc1)cn2C1CCCCC1 ; Cl ; Cl] | N-(3-(1-cyclohexyl-1H- indol-3-yl)-3-(pyridin-4- yl)propyl)piperidin- 4-amine hydrochloride salt | 489.53 | 416.29 | 417.3 | I |
| 336 | 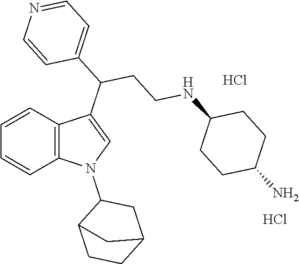 [SMILES: N[C@H]1CC[C@H](NCCC(c2ccncc2)c2cn(C3CC4CCC3C4)c3ccccc23)CC1 ; Cl ; Cl] | (1R,4R)-N1-(3-(1- (bicyclo[2.2.1] heptan-2-yl)- 1H-indol-3-yl)- 3-(pyridin-4- yl)propyl)cyclohexane- 1,4-diamine hydrochloride salt | 515.57 | 442.31 | 443.3 | I |
| 337 | 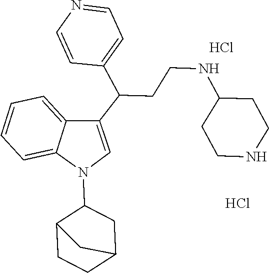 [SMILES: c1ccc2c(c1)c(C(CCNC1CCNCC1)c1ccncc1)cn2C1CC2CCC1C2 ; Cl ; Cl] | N-(3-(1- (bicyclo[2.2.1] heptan-2-yl)- 1H-indol-3-yl)- 3-(pyridin-4- yl)propyl)piperidin- 4-amine hydrochloride salt | 501.54 | 428.29 | 429.3 | I |
| 338 | 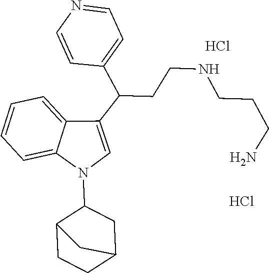 [SMILES: NCCCNCCC(c1ccncc1)c1cn(C2CC3CCC2C3)c2ccccc12 ; Cl ; Cl] | N1-(3-(1- (bicyclo[2.2.1] heptan-2-yl)- 1H-indol-3-yl)- 3-(pyridin-4- yl)propyl)propane-1,3- diamine hydrochloride salt | 475.5 | 402.28 | 403.2 | I |
| 339 | 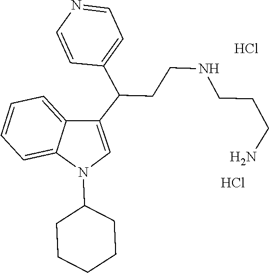 [SMILES: NCCCNCCC(c1ccncc1)c1cn(C2CCCCC2)c2ccccc12 ; Cl ; Cl] | N1-(3-(1- cyclohexyl-1H- indol-3-yl)-3-(pyridin-4- yl)propyl)propane-1,3- diamine hydrochloride salt | 463.49 | 390.28 | 391.2 | I |
| 340 | 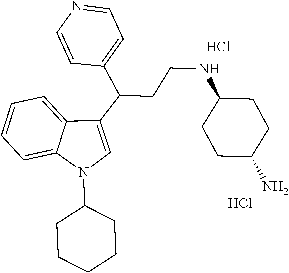 [SMILES: N[C@H]1CC[C@H](NCCC(c2ccncc2)c2cn(C3CCCCC3)c3ccccc23)CC1 ; Cl ; Cl] | (1r,4r)-N1-(3-(1- cyclohexyl-1H- indol-3-yl)-3- (pyridin-4-yl)propyl) cyclohexane-1,4- diamine hydrochloride salt | 503.56 | 430.31 | 431.3 | I |
| 341 | 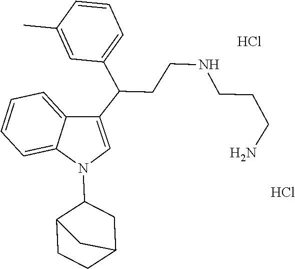 [SMILES: Cc1cccc(C(CCNCCCN)c2cn(C3CC4CCC3C4)c3ccccc23)c1 ; Cl ; Cl] | N1-(3-(1- (bicyclo[2.2.1] heptan-2-yl)- 1H-indol-3-yl)-3-(m- tolyl)propyl)propane- 1,3-diamine hydrochloride salt | 488.54 | 415.30 | | I |
| 342 | 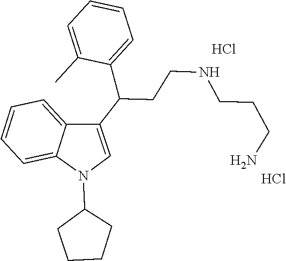 [SMILES: Cc1ccccc1C(CCNCCCN)c1cn(C2CCCC2)c2ccccc12 ; Cl ; Cl] | N1-(3-(1-cyclopentyl- 1H-indol-3-yl)-3-(o- tolyl)propyl)propane- 1,3- diamine hydrochloride salt | 462.5 | 389.28 | 390.2 | I |
| 343 | 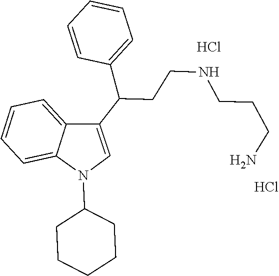 [SMILES: NCCCNCCC(c1ccccc1)c1cn(C2CCCCC2)c2ccccc12 ; Cl ; Cl] | N1-(3-(1-cyclohexyl- 1H- indol-3-yl)-3- phenylpropyl) propane-1,3- diamine hydrochloride salt | 462.5 | 398.28 | 390.2 | I |
| 344 | 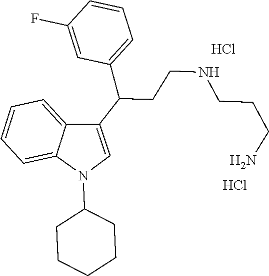 [SMILES: NCCCNCCC(c1cccc(F)c1)c1cn(C2CCCCC2)c2ccccc12 ; Cl ; Cl] | N1-(3-(1-cyclohexyl- 1H-indol-3-yl)-3-(3- fluorophenyl)propyl) propane-1,3-diamine hydrochloride salt | 480.49 | | 408.2 | I |
| 345 | 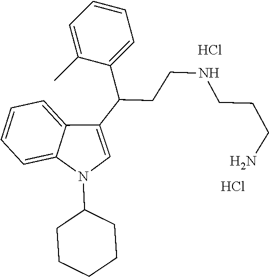 [SMILES: Cc1ccccc1C(CCNCCCN)c1cn(C2CCCCC2)c2ccccc12 ; Cl ; Cl] | N1-(3-(1-cyclohexyl- 1H- indol-3-yl)-3-(o- tolyl)propyl)propane- 1,3- diamine hydrochloride salt | 476.53 | 407.27 | 404.3 | I |
| 346 | 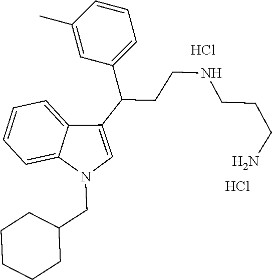 [SMILES: Cc1cccc(C(CCNCCCN)c2cn(CC3CCCCC3)c3ccccc23)c1 ; Cl ; Cl] | N1-(3-(1- (cyclohexylmethyl)- 1H-indol-3-yl)-3-(m- tolyl)propyl)propane- 1,3-diamine hydrochloride salt | 490.56 | 417.31 | 418.3 | II |
| 347 | 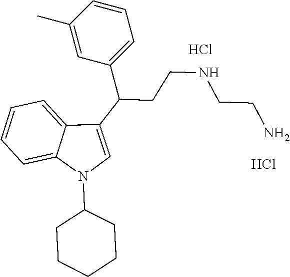 [SMILES: Cc1cccc(C(CCNCCN)c2cn(C3CCCCC3)c3ccccc23)c1 ; Cl ; Cl] | N1-(3-(1-cyclohexyl-1H- indol-3-yl)-3-(m- tolyl)propyl)ethane-1,2- diamine hydrochloride salt | 462.5 | 389.28 | 390.2 | I |
| 348 | 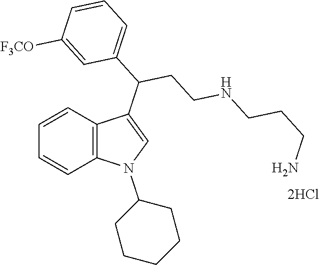 [SMILES: NCCCNCCC(c1cccc(OC(F)(F)F)c1)c1cn(C2CCCCC2)c2ccccc12 ; "2HCl"] | N1-(3-(1-cyclohexyl-1H- indol-3-yl)-3-(3- (trifluoromethoxy)phenyl) propyl)propane-1,3- diaminesalt | 546.5 | 473.27 | 474.2 | I |
| 349 | 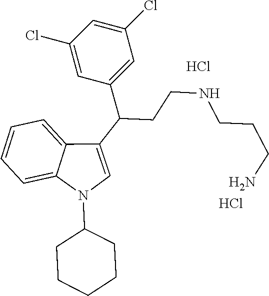 [SMILES: NCCCNCCC(c1cc(Cl)cc(Cl)c1)c1cn(C2CCCCC2)c2ccccc12 ; Cl ; Cl] | N1-(3-(1-cyclohexyl-1H- indol-3-yl)-3-(3,5- dichlorophenyl)propyl) propane-1,3-diamine hydrochloride salt | 531.39 | 457.21 | 458.2 | I |
| 350 | 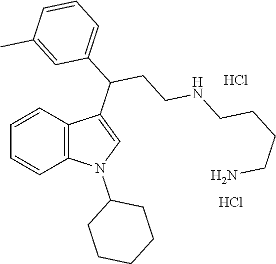 [SMILES: Cc1cccc(C(CCNCCCCN)c2cn(C3CCCCC3)c3ccccc23)c1 ; Cl ; Cl] | N1-(3-(1-cyclohexyl-1H- indol-3-yl)-3-(m- tolyl)propyl)butane-1,4- diamine salt | 490.56 | 417.31 | 418.3 | I |
| 351 | 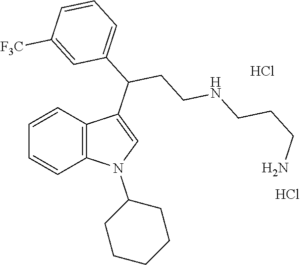 [SMILES: NCCCNCCC(c1cccc(C(F)(F)F)c1)c1cn(C2CCCCC2)c2ccccc12 ; Cl ; Cl] | N1-(3-(1-cyclohexyl-1H- indol-3-yl)-3-(3- (trifluoromethyl)phenyl) propyl)propane-1,3- diamine hydrochloride salt | 530.5 | 457.27 | 458.3 | I |
| 352 | 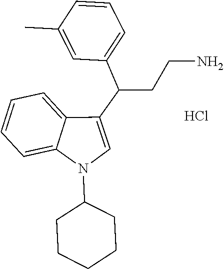 [SMILES: Cc1cccc(C(CCN)c2cn(C3CCCCC3)c3ccccc23)c1 ; Cl] | 3-(1-cyclohexyl- 1H-indol-3-yl)-3- (m-tolyl)propan-1- amine hydrochloride salt | 382.98 | 34.24 | 347.2 | I |
| 353 | 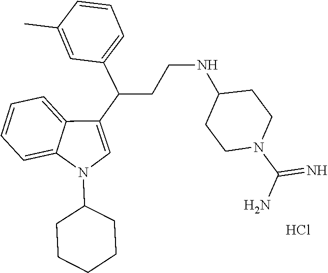 [SMILES: Cc1cccc(C(CCNC2CCN(C(=N)N)CC2)c2cn(C3CCCCC3)c3ccccc23)c1 ; Cl] | 4-((3-(1-cyclohexyl-1H- indol-3-yl)-3-(m-tolyl) propyl)amino)piperidine- 1-carboximidamide hydrochloride salt | 544.61 | 471.34 | 472.3 | I |
| 354 | 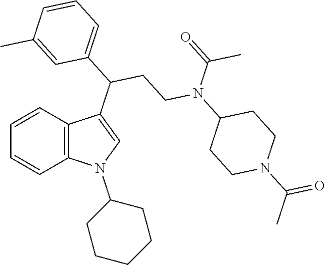 [SMILES: CC(=O)N1CCC(N(CCC(c2cccc(C)c2)c2cn(C3CCCCC3)c3ccccc23)C(C)=O)CC1] | N-(1-acetylpiperidin- 4-yl)-N-(3-(1- cyclohexyl-1H- indol-3-yl)-3-(m- tolyl)propyl)acetamide salt | 513.73 | 513.34 | 514.3 | I |
| 655 | 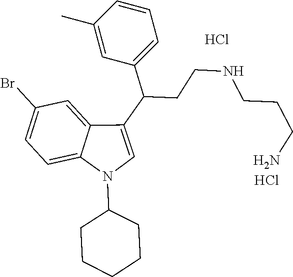 [SMILES: Cc1cccc(C(CCNCCCN)c2cn(C3CCCCC3)c3ccc(Br)cc23)c1 ; Cl ; Cl] | N1-(3-(5-bromo-1- cyclohexyl-1H- indol-3-yl)-3-(m- tolyl)propyl)propane- 1,3-diamine hydrochloride salt | 555.43 | 481.21 | 482.2 | I |
| 356 | 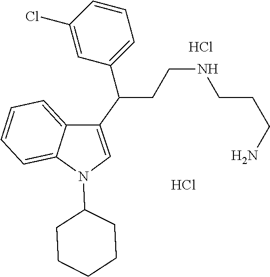 [SMILES: NCCCNCCC(c1cccc(Cl)c1)c1cn(C2CCCCC2)c2ccccc12 ; Cl ; Cl] | N1-(3-(3- chlorophenyl)-3- (1-cyclohexyl-1H- indol-3-yl)propyl) propane-1,3- diamine hydrochloride salt | 496.95 | 423.24 | 424.3 | I |
| 357 | 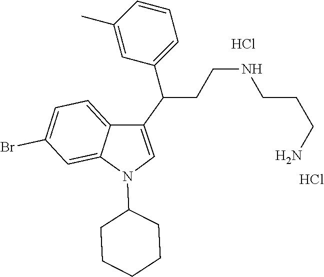 [SMILES: Cc1cccc(C(CCNCCCN)c2cn(C3CCCCC3)c3cc(Br)ccc23)c1 ; Cl ; Cl] | N1-(3-(6-bromo-1- cyclohexyl-1H- indol-3-yl)- 3-(m-tolyl) propyl)propane- 1,3-diamine hydrochloride salt | 555.43 | 481.21 | 484.2 | I |
| 358 | 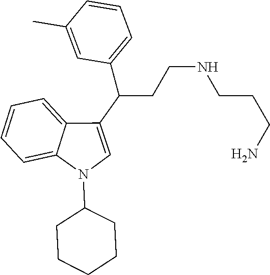 [SMILES: Cc1cccc(C(CCNCCCN)c2cn(C3CCCCC3)c3ccccc23)c1] | N1-(3-(1-cyclohexyl- 1H-indol-3-yl)-3-(m- tolyl)propyl)propane-1,3- diamine | NA | 403.61 | 404.3 | I |
| 359 | 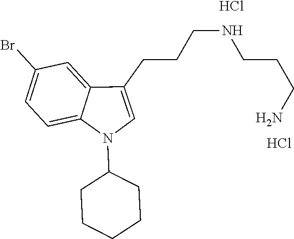 [SMILES: Cl ; NCCCNCCCc1cn(C2CCCCC2)c2ccc(Br)cc12 ; Cl] | N1-(3-(5-bromo-1- cyclohexyl-1H-indol-3- yl)propyl)propane-1,3- diamine hydrochloride salt | 465.3 | 391.16 | 392.2 | I |
| 360 | 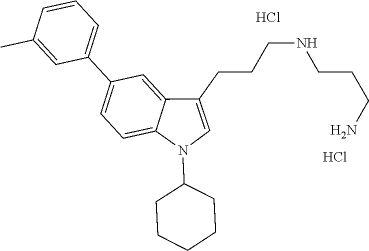 [SMILES: Cc1cccc(-c2ccc3c(c2)c(CCCNCCCN)cn3C2CCCCC2)c1 ; Cl ; Cl] | N1-(3-(1-cyclohexyl-5- (m-tolyl)-1H-indol-3- yl)propyl)propane-1,3- diamine hydrochloride salt | 476.53 | 403.30 | 404.3 | III |
| 361 | 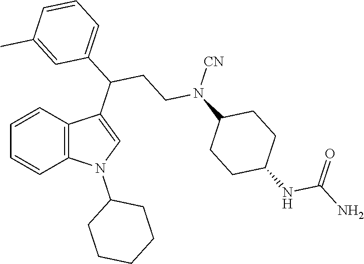 | 1-(((1S,4S)-4-(N-(3-(1- cyclohexyl-1H- indol-3-yl)- 3-(m- tolyl)propyl)cyanamido) cyclohexyl)urea salt | 511.71 | 511.33 | 512.3 | I |
| 362 | 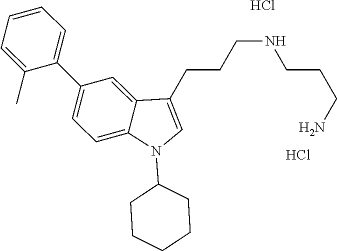 [SMILES: Cl ; Cc1ccccc1-c1ccc2c(c1)c(CCCNCCCN)cn2C1CCCCC1 ; Cl] | N1-(3-(1-cyclohexyl- 5-(o-tolyl)-1H-indol-3- yl)propyl)propane-1,3- diamine hydrochloride salt | 476.53 | 403.30 | 404.3 | III |
| 363 | 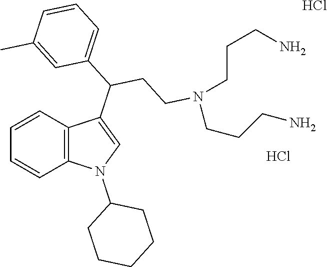 [SMILES: Cc1cccc(C(CCN(CCCN)CCCN)c2cn(C3CCCCC3)c3ccccc23)c1 ; Cl ; Cl] | N1-(3-aminopropyl)- N1-(3-(1-cyclohexyl- 1H-indol-3-yl)-3-(m- tolyl)propyl)propane- 1,3-diamine hydrochloride salt | 533.63 | 460.36 | 461.3 | I |
| 364 | 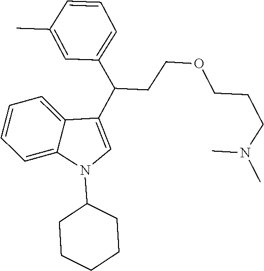 [SMILES: Cc1cccc(C(CCOCCCN(C)C)c2cn(C3CCCCC3)c3ccccc23)c1] | 3-(3-(1-cyclohexyl-1H- indol-3-yl)-3-(m- tolyl)propoxy)-N,N- dimethylpropan-1-amine salt | 432.65 | 432.31 | 433.3 | I |
| 365 | 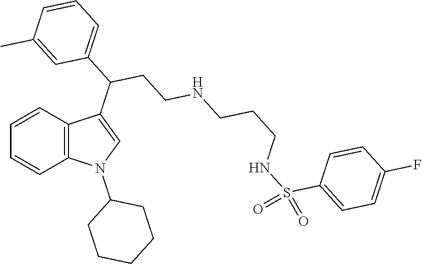 [SMILES: Cc1cccc(C(CCNCCCNS(=O)(=O)c2ccc(F)cc2)c2cn(C3CCCCC3)c3ccccc23)c1] | N-(3-((3-(1-cyclohexyl- 1H-indol-3-yl)-3-(m- tolyl)propyl)amino) propyl)- 4- fluorobenzenesulfonamide | NA | 561.76 | 562.3 | I |
| 366 | 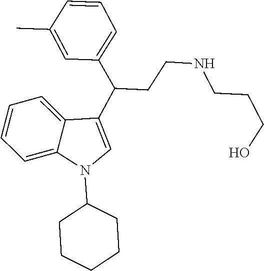 [SMILES: Cc1cccc(C(CCNCCCO)c2cn(C3CCCCC3)c3ccccc23)c1] | 3-((3-(1-cyclohexyl- 1H-indol-3-yl)-3-(m- tolyl)propyl)amino) propan-1-ol | NA | 404.6 | 458.3 | I |
| 367 | 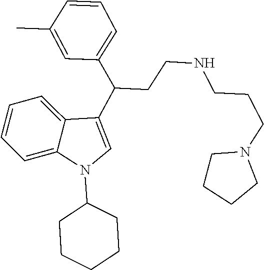 [SMILES: Cc1cccc(C(CCNCCCN2CCCC2)c2cn(C3CCCCC3)c3ccccc23)c1] | 3-(1-cyclohexyl- 1H-indol-3- yl)-N-(3-(pyrrolidin-1- yl)propyl)-3-(m- tolyl)propan-1-amine | NA | 457.71 | 458.3 | I |
| 368 | 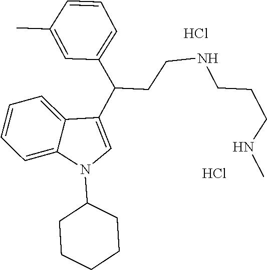 [SMILES: CNCCCNCCC(c1cccc(C)c1)c1cn(C2CCCCC2)c2ccccc12 ; Cl ; Cl] | N1-(3-(1-cyclohexyl- 1H-indol-3-yl)-3-(m- tolyl)propyl)-N3- methylpropane- 1,3-diamine hydrochloride | 490.56 | 417.31 | 418.3 | I |
| 369 | 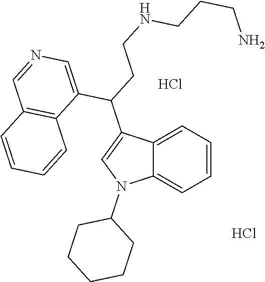 [SMILES: NCCCNCCC(c1cncc2ccccc12)c1cn(C2CCCCC2)c2ccccc12 ; Cl ; Cl] | N1-(3-(1-cyclohexyl- 1H-indol-3-yl)-3- (isoquinolin-4- yl)propyl)propane-1,3- diamine hydrochloride | 513.56 | 440.29 | 441.3 | I |
| 370 | 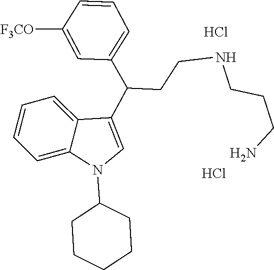 [SMILES: NCCCNCCC(c1cccc(OC(F)(F)F)c1)c1cn(C2CCCCC2)c2ccccc12 ; Cl ; Cl] | N1-(3-(1-cyclohexyl- 1H-indol-3-yl)-3-(3- (trifluoromethoxy) phenyl)propyl) propane- 1,3-diamine salt | 546.5 | 473.27 | 473.2 | I |
| 371 | 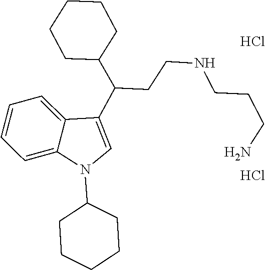 [SMILES: NCCCNCCC(c1cn(C2CCCCC2)c2ccccc12)C1CCCCC1 ; Cl ; Cl] | N1-(3-cyclohexyl-3-(1- cyclohexyl-1H-indol-3- yl)propyl)propane-1,3- diamine hydrochloride | 468.55 | 395.33 | 396.3 | I |
| 372 | 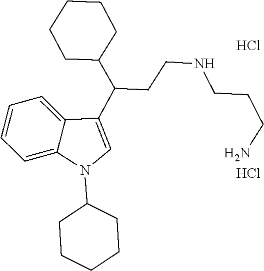 [SMILES: NCCCNCCC(c1cn(C2CCCCC2)c2ccccc12)C1CCCCC1 ; Cl ; Cl] | N1-(3-cyclohexyl-3-(1- cyclohexyl-1H-indol-3- yl)propyl)propane-1,3- diamine dihydrochloride salt | 571.51 | 395.33 | 499.3 | I |
| 373 | 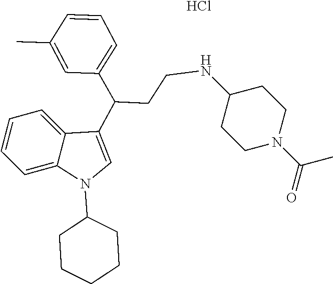 [SMILES: Cl ; CC(=O)N1CCC(NCCC(c2cccc(C)c2)c2cn(C3CCCCC3)c3ccccc23)CC1] | 1-(4-((3-(1- cyclohexyl-1H- indol-3-yl)-3-(m- tolyl)propyl)amino) piperidin-1-yl) ethan-1-one salt | 508.15 | 471.32 | 472.2 | I |
| 374 | 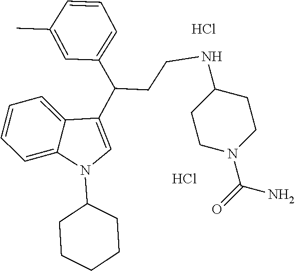 [SMILES: Cc1cccc(C(CCNC2CCN(C(N)=O)CC2)c2cn(C3CCCCC3)c3ccccc23)c1 ; Cl ; Cl] | 4-((3-(1-cyclohexyl- 1H-indol-3-yl)-3-(m- tolyl)propyl) amino)piperidine- 1-carboxamide hydrochloride salt | 509.14 | 472.32 | 473.3 | I |
| 375 | 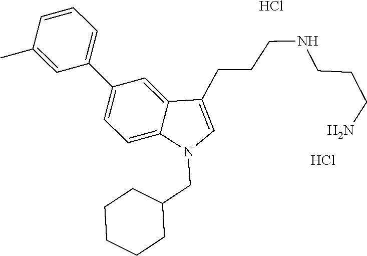 [SMILES: Cl ; Cc1cccc(-c2ccc3c(c2)c(CCCNCCCN)cn3CC2CCCCC2)c1 ; Cl] | N1-(3-(1- (cyclohexylmethyl)- 5-(m-tolyl)-1H-indol-3- yl)propyl)propane-1,3- diamine hydrochloride salt | 490.56 | 417.31 | 418.3 | III |
| 376 | 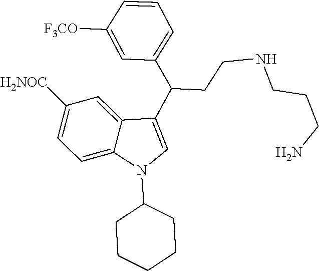 [SMILES: NCCCNCCC(c1cccc(OC(F)(F)F)c1)c1cn(C2CCCCC2)c2ccc(C(N)=O)cc12] | 3-(3-((3- aminopropyl) amino)-1-(3- (trifluoromethoxy) phenyl)propyl)-1- cyclohexyl-1H- indole-5-carboxamide | NA | 516.61 | 517.2 | I |
| 377 | 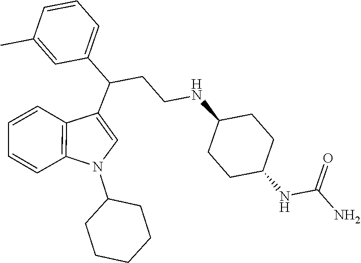 [SMILES: Cc1cccc(C(CCN[C@H]2CC[C@H](NC(N)=O)CC2)c2cn(C3CCCCC3)c3ccccc23)c1] | 1-((1R,4R)-4-((3- (1-cyclohexyl-1H- indol-3-yl)-3-(m- tolyl)propyl)amino) cyclohexyl) urea | NA | 486.7 | 487.3 | I |
| 378 | 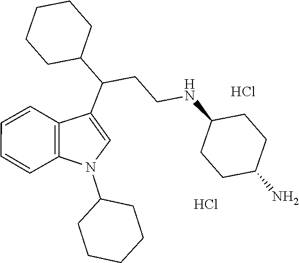 [SMILES: N[C@H]1CC[C@H](NCCC(c2cn(C3CCCCC3)c3ccccc23)C2CCCCC2)CC1 ; Cl ; Cl] | ((1R,4R)-N1-(3- cyclohexyl- 3-(1-cyclohexyl- 1H-indol-3- yl)propyl) cyclohexane-1,4- diamine salt | 508.62 | 435.36 | 436.3 | I |
| 379 | 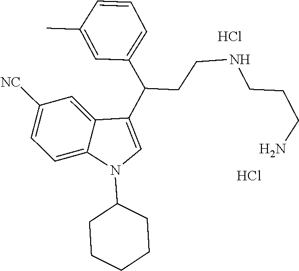 [SMILES: Cc1cccc(C(CCNCCCN)c2cn(C3CCCCC3)c3ccc(C#N)cc23)c1 ; Cl ; Cl] | 3-(3-((3- aminopropyl) amino)-1-(m- tolyl)propyl)-1- cyclohexyl- 1H-indole-5- carbonitrile hydrochloride salt | 501.45 | 428.29 | 429.45 | I |
| 380 | 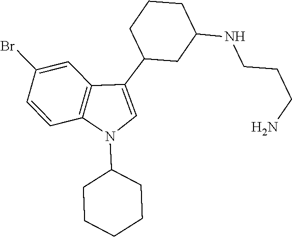 [SMILES: NCCCNC1CCCC(c2cn(C3CCCCC3)c3ccc(Br)cc23)C1] | N1-(3-(5-bromo-1- cyclohexyl-1H-indol-3- yl)cyclohexyl)propane- 1,3-diamine salt | NA | 432.15 | 432.2 | I |
| 381 | 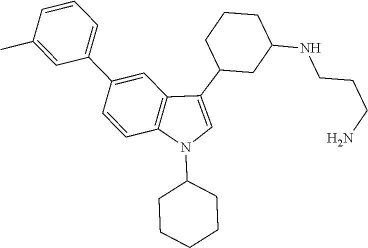 [SMILES: Cc1cccc(-c2ccc3c(c2)c(C2CCCC(NCCCN)C2)cn3C2CCCCC2)c1] | N1-(3-(1-cyclohexyl-5- (m- tolyl)-1H-indol-3- yl)cyclohexyl)propane- 1,3-diamine | NA | 443.68 | 444.2 | III |
| 382 | 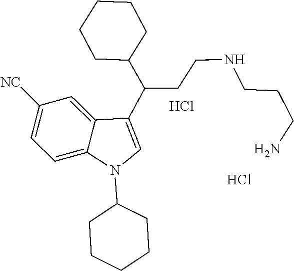 [SMILES: N#Cc1ccc2c(c1)c(C(CCNCCCN)C1CCCCC1)cn2C1CCCCC1 ; Cl ; Cl] | 3-(3-((3 - aminopropyl)amino)-1- cyclohexylpropyl)-1- cyclohexyl-1H-indole-5- carbonitrile hydrochloride salt | 493.56 | 420.33 | 421.5 | I |
| 383 | 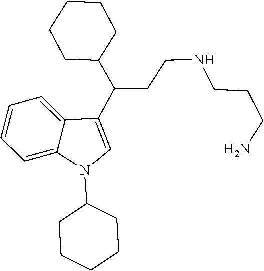 [SMILES: NCCCNCCC(c1cn(C2CCCCC2)c2ccccc12)C1CCCCC1] | N1-(3-cyclohexyl-3-(1- cyclohexyl-1H-indol-3- yl)propyl)propane-1,3- diamine | NA | 395.64 | 396.51 | I |
| 384 | 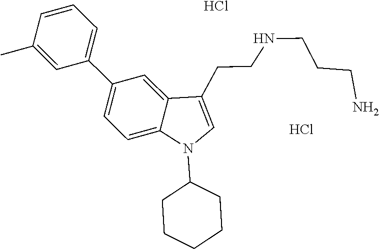 [SMILES: Cc1cccc(-c2ccc3c(c2)c(CCNCCCN)cn3C2CCCCC2)c1 ; Cl ; Cl] | N1-(2-(1-cyclohexyl-5- (m-tolyl)-1H-indol-3- yl)ethyl)propane-1,3- diamine hydrochloride salt | 461.22 | 389.28 | 390.44 | III |
| 385 | 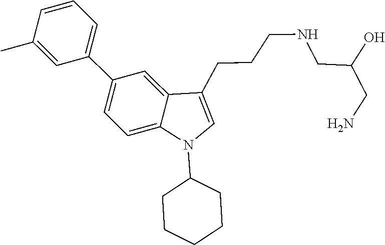 [SMILES: Cc1cccc(-c2ccc3c(c2)c(CCCNCC(O)CN)cn3C2CCCCC2)c1] | 1-amino-3-((3-(1- cyclohexyl-5-(m- tolyl)-1H- indol-3- yl)propyl)amino) propan-2-ol | NA | 419.29 | 420.51 | III |
| 386 | 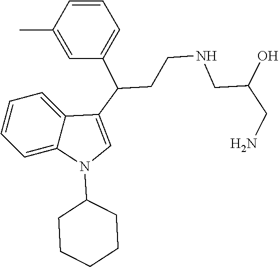 [SMILES: Cc1cccc(C(CCNCC(O)CN)c2cn(C3CCCCC3)c3ccccc23)c1] | 1-amino-3-((3-(1- cyclohexyl-1H- indol-3-yl)- 3-(m- tolyl)propyl)amino) propan- 2-ol | NA | 419.29 | 420.46 | I |
| 387 | 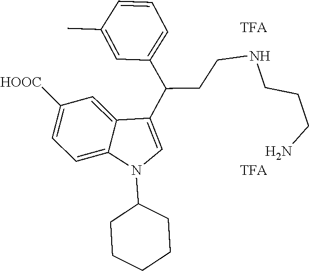 [SMILES: Cc1cccc(C(CCNCCCN)c2cn(C3CCCCC3)c3ccc(C(=O)O)cc23)c1] | 3-(3-((3- aminopropyl) amino)-1-(m- tolyl)propyl)-1- cyclohexyl- 1H-indole-5- carboxylic acid compound with 2,2,2- trifluoroacetaldehyde (1:2) | 675.27 | 447.29 | 448.53 | I |
| 388 | 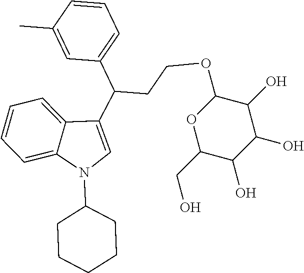 [SMILES: Cc1cccc(C(CCOC2OC(CO)C(O)C(O)C2O)c2cn(C3CCCCC3)c3ccccc23)c1] | 2-(3-(1-cyclohexyl-1H- indol-3-yl)-3-(m- tolyl)propoxy)-6- (hydroxymethyl) tetrahydro- 2H-pyran-3,4,5-triol | NA | 509.28 | 510.3 | I |
| 389 | 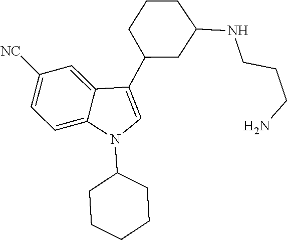 [SMILES: N#Cc1ccc2c(c1)c(C1CCCC(NCCCN)C1)cn2C1CCCCC1] | 3-(3-((3- aminopropyl) amino)cyclohexyl)- 1-cyclohexyl-1H- indole-5-carbonitrile | NA | 378.28 | 379.4 | IV |
| 390 | 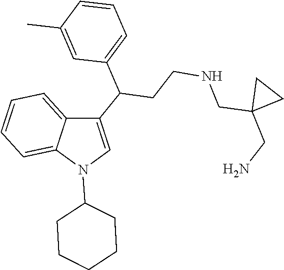 [SMILES: Cc1cccc(C(CCNCC2(CN)CC2)c2cn(C3CCCCC3)c3ccccc23)c1] | N-((1- (aminomethyl) cyclopropyl) methyl)-3-(1-cyclohexyl- 1H-indol-3-yl)-3-(m- tolyl)propan-1-amine | NA | 429.31 | 430.5 | I |
| 391 | 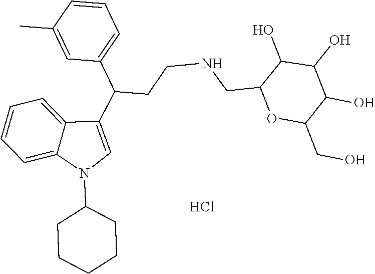 [SMILES: Cc1cccc(C(CCNCC2OC(CO)C(O)C(O)C2O)c2cn(C3CCCCC3)c3ccccc23)c1 ; Cl] | 2-(((3-(1-cyclohexyl- 1H- indol-3-yl)-3-(m- tolyl)propyl) amino)methyl)-6- (hydroxymethyl) tetrahydro- 2H-pyran-3,4,5-triol | NA | 522.61 | 523.5 | I |
| 392 | 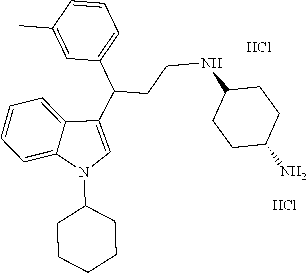 | (1S,4S)- -N1-(3-(1- cyclohexyl-1H- indol-3-yl)-3-(m- tolyl)propyl) cyclohexane- 1,4-diamine hydrochloride salt | 515.28 | 443.33 | 444.5 | I |
| 393 | 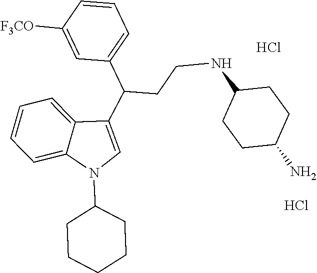 | (1S,4S)- -N1-(3-(1- cyclohexyl-1H- indol-3-yl)- 3-(3- (trifluoromethoxy) phenyl) propyl)cyclohexane- 1,4-diamine hydrochloride salt | 585.25 | 513.30 | 514.5 | I |
| 394 | 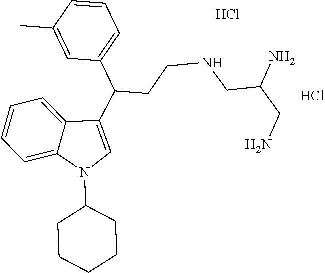 [SMILES: Cc1cccc(C(CCNCC(N)CN)c2cn(C3CCCCC3)c3ccccc23)c1 ; Cl ; Cl] | N1-(3-(1-cyclohexyl- 1H-indol-3-yl)-3-(m- tolyl)propyl)propane- 1,2,3-triamine hydrochloride salt | 526.14 | 418.31 | 419.3 | I |
| 395 | 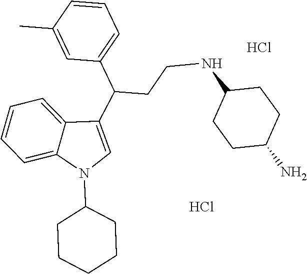 | (1S,4S)- -N1-(3-(1- cyclohexyl-1H-indol- 3-yl)-3-(m- tolyl)propyl) cyclohexane- 1,4-diamine hydrochloride salt | 555.31 | 443.33 | 484.4 | I |
| 396 | 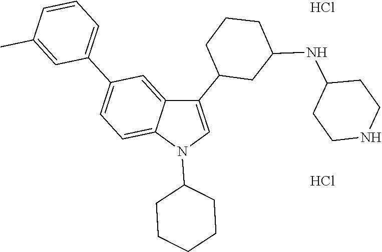 [SMILES: Cc1cccc(-c2ccc3c(c2)c(C2CCCC(NC4CCNCC4)C2)cn3C2CCCCC2)c1 ; Cl ; Cl] | N-(3-(1-cyclohexyl-5-(m- tolyl)-1H-indol-3-yl) cyclohexyl)piperidin-4- amine hydrochloride salt | 541.3 | 469.35 | 470.3 | IV |
| 397 | 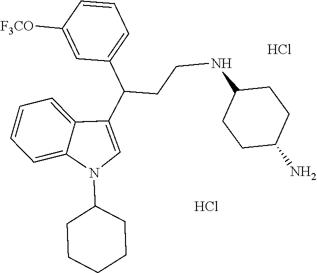 | (1S,4S)-N1-(3-(1- cyclohexyl-1H- indol-3-yl)-3-(3- (trifluoromethoxy)phenyl) propyl)cyclohexane-1,4- diamine hydrochloride salt | 625.28 | 513.30 | 544.4 | I |
| 398 | 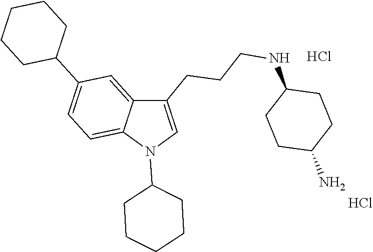 [SMILES: N[C@H]1CC[C@H](NCCCc2cn(C3CCCCC3)c3ccc(C4CCCCC4)cc23)CC1 ; Cl ; Cl] | (1r,4r)-N1-(3-(1,5- dicyclohexyl- 1H-indol-3- yl)propyl)cyclohexane- 1,4-diamine hydrochloride salt | 507.31 | 435.36 | 436.3 | III |
| 399 | 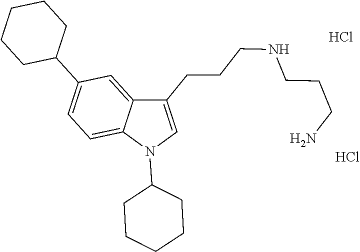 [SMILES: NCCCNCCCc1cn(C2CCCCC2)c2ccc(C3CCCCC3)cc12 ; Cl ; Cl] | N1-(3-(1,5- dicyclohexyl- 1H-indol-3- yl)propyl)propane-1,3- diamine hydrochloride salt | 467.28 | 395.33 | 396.3 | III |
| 400 | 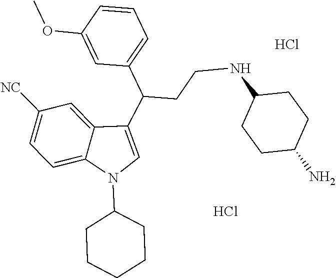 | 3-(3-(((1s,4s)-4- aminocyclohexyl) amino)-1-(3- methoxyphenyl) propyl)-1-cyclohexyl- 1H-indole-5- carbonitrile hydrochloride salt | 556.27 | 484.32 | 488.2 | I |
| 401 | 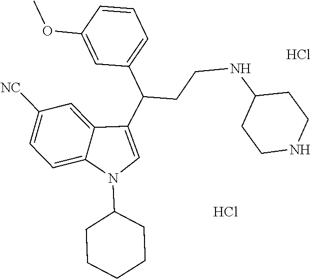 [SMILES: COc1cccc(C(CCNC2CCNCC2)c2cn(C3CCCCC3)c3ccc(C#N)cc23)c1 ; Cl ; Cl] | 1-cyclohexyl-3-(1-(3- methoxyphenyl)-3- (piperidin-4- ylamino)propyl)- 1H-indole- 5-carbonitrile hydrochloride salt | 542.26 | 470.30 | 471.3 | I |
| 402 | 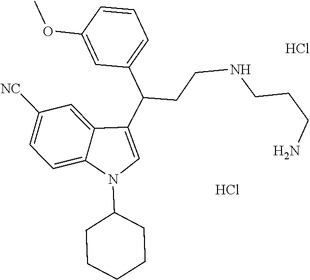 [SMILES: COc1cccc(C(CCNCCCN)c2cn(C3CCCCC3)c3ccc(C#N)cc23)c1 ; Cl ; Cl] | 3-(3-((3- aminopropyl) amino)-1-(3- methoxyphenyl) propyl)-1- cyclohexyl-1H- indole-5- carbonitrile hydrochloride salt | 516.24 | 444.29 | 445.3 | I |
| 403 | 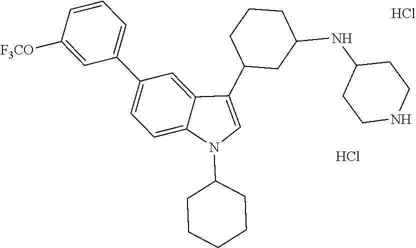 [SMILES: FC(F)(F)Oc1cccc(-c2ccc3c(c2)c(C2CCCC(NC4CCNCC4)C2)cn3C2CCCCC2)c1 ; Cl ; Cl] | N-(3-(1-cyclohexyl- 5-(3-(trifluoromethoxy) phenyl)-1H-indol-3- yl)cyclohexyl) piperidin-4- amine hydrochloride salt | 611.27 | 539.31 | 540.3 | IV |
| 404 | 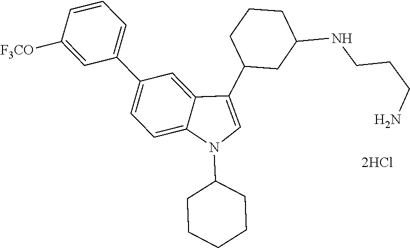 [SMILES: NCCCNC1CCCC(c2cn(C3CCCCC3)c3ccc(-c4cccc(OC(F)(F)F)c4)cc23)C1 ; "2HCl"] | N1-(3-(1- cyclohexyl-5-(3- (trifluoromethoxy) phenyl)- 1H-indol-3- yl)cyclohexyl)propane- 1,3- diamine hydrochloride salt | 585.25 | 523.30 | 514.33 | IV |
| 405 | 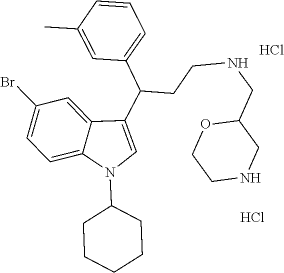 [SMILES: Cc1cccc(C(CCNCC2CNCCO2)c2cn(C3CCCCC3)c3ccc(Br)cc23)c1 ; Cl ; Cl] | 3-(5-bromo-1- cyclohexyl- 1H-indol-3-yl)-N- (morpholin-2-ylmethyl)- 3-(m-tolyl)propan- 1-amine hydrochloride salt | 595.17 | 523.22 | 524.3 | I |
| 406 | 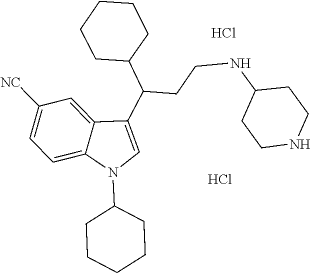 [SMILES: N#Cc1ccc2c(c1)c(C(CCNC1CCNCC1)C1CCCCC1)cn2C1CCCCC1 ; Cl ; Cl] | 1-cyclohexyl-3-(1- cyclohexyl-3-(piperidin-4- ylamino)propyl)-1H-indole- 5-carbonitrile salt | 518.29 | 446.34 | 447.4 | I |
| 407 | 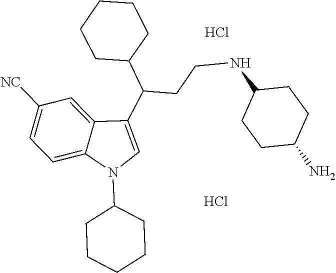 | 3-(3-(((1R,4R)-4- aminocyclohexyl) amino)-1- cyclohexylpropyl)-1- cyclohexyl-1H-indole- 5-carbonitrile salt | 532.31 | 460.36 | 461.3 | I |
| 408 | 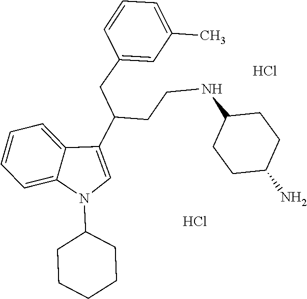 | (1R,4R)-N1-(3-(1- cyclohexyl-1H- indol-3-yl)-4-(m- tolyl)butyl)cyclohexane- 1,4-diamine hydrochloride salt | 529.3 | 457.3 | 458.39 | I |
| 409 | 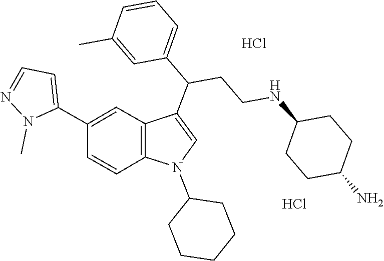 [SMILES: Cc1cccc(C(CCN[C@H]2CC[C@H](N)CC2)c2cn(C3CCCCC3)c3ccc(-c4ccnn4C)cc23)c1 ; Cl ; Cl] | (1R,4R)-N1-(3-(1- cyclohexyl-5-(1-methyl- 1H-pyrazol-5-yl)-1H- indol-3-yl)-3-(m- tolyl)propyl)cyclohexane- 1,4-diamine hydrochloride salt | 595.32 | 523.37 | 524.39 | III |
| 410 | 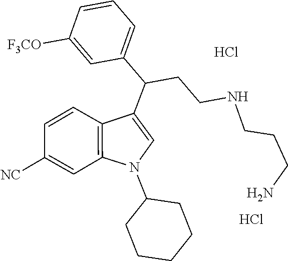 [SMILES: N#Cc1ccc2c(C(CCNCCCN)c3cccc(OC(F)(F)F)c3)cn(C3CCCCC3)c2c1 ; Cl ; Cl] | 3-(3-((3- aminopropyl)amino)-1-(3- (trifluoromethoxy)phenyl) propyl)-1-cyclohexyl-1H- indole-6-carbonitrile salt | 500.21 | 498.26 | 499.33 | 1 |
| 412 | 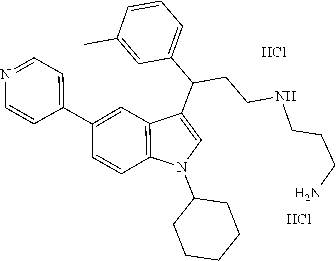 [SMILES: Cc1cccc(C(CCNCCCN)c2cn(C3CCCCC3)c3ccc(-c4ccncc4)cc23)c1 ; Cl ; Cl] | N1-(3-(1-cyclohexyl-5- (pyridin-4-yl)- 1H-indol-3- yl)-3-(m- tolyl)propyl) propane-1,3- diamine hydrochloride salt | 552.28 | 480.33 | 481.35 | III |
| 413 | 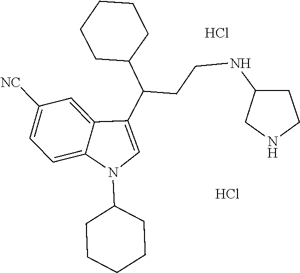 [SMILES: N#Cc1ccc2c(c1)c(C(CCNC1CCNC1)C1CCCCC1)cn2C1CCCCC1 ; Cl ; Cl] | 1-cyclohexyl-3-(1- cyclohexyl-3- (pyrrolidin-3- ylamino)propyl)- 1H-indole- 5-carbonitrile hydrochloride salt | 504.28 | 432.22 | 433.3 | I |
| 414 | 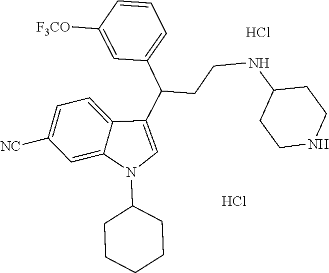 [SMILES: N#Cc1ccc2c(C(CCNC3CCNCC3)c3cccc(OC(F)(F)F)c3)cn(C3CCCCC3)c2c1 ; Cl ; Cl] | 1-cyclohexyl-3-(3- (piperidin-4- ylamino)-1-(3- (trifluoromethoxy)phenyl) propyl)-1H-indole-6- carbonitrile hydrochloride salt | 596.23 | 524.28 | 525.32 | I |
| 415 | 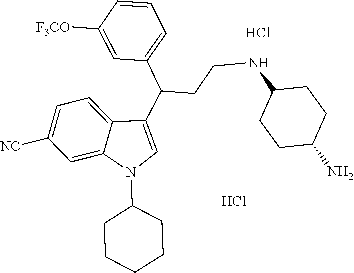 | 3-(3-(((1S,4S)-4- aminocyclohexyl) amino)-1-(3- (trifluoromethoxy) phenyl)propyl)-1- cyclohexyl-1H- indole-6-carbonitrile hydrochloride salt | 610.25 | 538.29 | 539.2 | I |
| 416 | 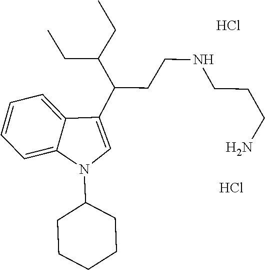 [SMILES: CCC(CC)C(CCNCCCN)c1cn(C2CCCCC2)c2ccccc12 ; Cl ; Cl] | N1-(3-(1-cyclohexyl-1H- indol-3-yl)-4- ethylhexyl)propane-1,3- diamine hydrochloride salt | 455.28 | 383.33 | 384.3 | I |
| 417 | 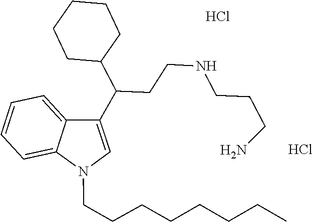 [SMILES: CCCCCCCCn1cc(C(CCNCCCN)C2CCCCC2)c2ccccc21 ; Cl ; Cl] | N1-(3-cyclohexyl-3-(1- octyl-1H-indol-3- yl)propyl)propane-1,3- diamine hydrochloride salt | 497.33 | 425.38 | 426.4 | I |
| 418 | 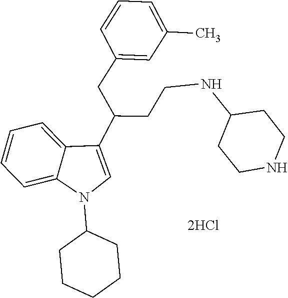 [SMILES: Cc1cccc(CC(CCNC2CCNCC2)c2cn(C3CCCCC3)c3ccccc23)c1 ; "2HCl"] | N-(3-(1-cyclohexyl-1H- indol-3-yl)-4-(m- tolyl)butyl)piperidin-4- amine hydrochloride salt | 443.33 | 443.33 | 444.3 | I |
| 419 | 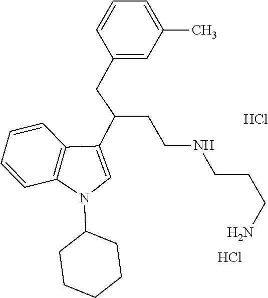 [SMILES: Cc1cccc(CC(CCNCCCN)c2cn(C3CCCCC3)c3ccccc23)c1 ; Cl ; Cl] | N1-(3-(1-cyclohexyl-1H- indol-3-yl)-4-(m- tolyl)butyl)propane-1,3- diamine hydrochloride salt | 489.27 | 417.31 | 418.3 | I |
| 420 | 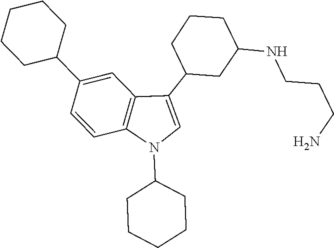 [SMILES: NCCCNC1CCCC(c2cn(C3CCCCC3)c3ccc(C4CCCCC4)cc23)C1] | N1-(3-(1,5-dicyclohexyl- 1H-indol-3- yl)cyclohexyl)propane- 1,3-diamine | NA | 435.36 | 436.4 | III |
| 421 | 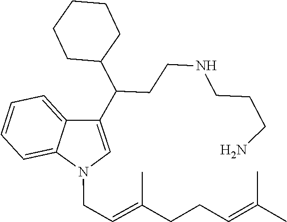 [SMILES: CC(C)=CCC/C(C)=C/Cn1cc(C(CCNCCCN)C2CCCCC2)c2ccccc21] | (E)-N1-(3-cyclohexyl- 3-(1-(3,7- dimethylocta-2,6-dien- 1-yl)-1H-indol-3- yl)propyl)propane-1,3- diamine | NA | 449.38 | 450.51 | I |
| 422 | 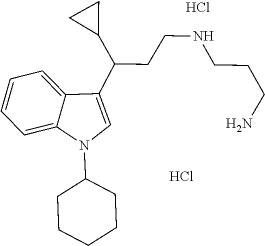 [SMILES: NCCCNCCC(c1cn(C2CCCCC2)c2ccccc12)C1CC1 ; Cl ; Cl] | N1-(3-(1-cyclohexyl- 1H- indol-3-yl)-3- cyclopropylpropyl) propane-1,3-diamine hydrochloride salt | 425.24 | 353.28 | 354.2 | I |
| 423 | 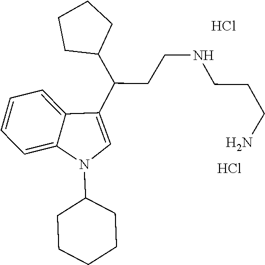 [SMILES: NCCCNCCC(c1cn(C2CCCCC2)c2ccccc12)C1CCCC1 ; Cl ; Cl] | N1-(3-(1-cyclohexyl- 1H- indol-3-yl)-3- cyclopentylpropyl) propane- 1,3-diamine hydrochloride salt | 453.24 | 381.31 | 382.39 | I |
| 424 | 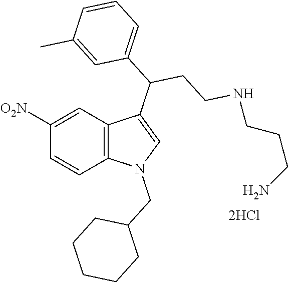 [SMILES: Cc1cccc(C(CCNCCCN)c2cn(CC3CCCCC3)c3ccc([N+](=O)[O-])cc23)c1] | N1-(3-(1- cyclohexylmethyl)- 5-nitro- 1H-indol-3-yl)-3-(m- tolyl)propyl)propane-1,3- diamine hydrochloride salt | 534.25 | 462.30 | 463.35 | I |
| 425 | 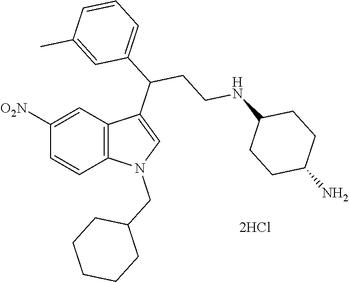 | (1S,4S)-N1-(3-(1- (cyclohexylmethyl)- 5-nitro- 1H-indol-3-yl)-3-(m- tolyl)propyl) cyclohexane- 1,4-diamine hydrochloride salt | 574.28 | 502.33 | 503.34 | I |
| 426 | 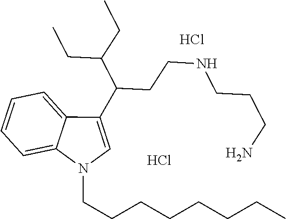 [SMILES: CCCCCCCCn1cc(C(CCNCCCN)C(CC)CC)c2ccccc21 ; Cl ; Cl] | N1-(4-ethyl-3- (1-octyl-1H- indol-3-yl)hexyl)propane- 1,3-diamine hydrochloride salt | 485.33 | 413.38 | 414.59 | I |
| 427 | 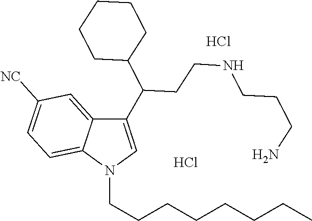 [SMILES: CCCCCCCCn1cc(C(CCNCCCN)C2CCCCC2)c2cc(C#N)ccc21 ; Cl ; Cl] | 3-(3-((3- aminopropyl)amino)-1- cyclohexylpropyl)- 1-octyl-1H-indole- 5-carbonitrile hydrochloride salt | 522.33 | 450.37 | 451.39 | I |
| 428 | 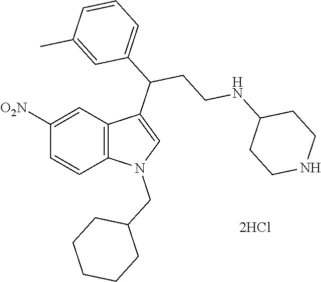 [SMILES: Cc1cccc(C(CCNC2CCNCC2)c2cn(CC3CCCCC3)c3ccc([N+](=O)[O-])cc23)c1] | N-(3-(1- (cyclohexylmethyl)- 5-nitro-1H-indol- 3-yl)-3-(m- tolyl)propyl)piperidin-4- amine hydrochloride salt | 560.27 | 488.32 | 489.39 | I |
| 429 | 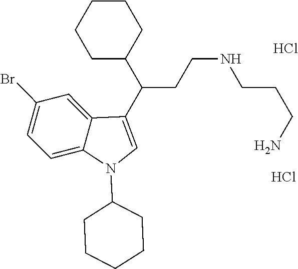 [SMILES: NCCCNCCC(c1cn(C2CCCCC2)c2ccc(Br)cc12)C1CCCCC1 ; Cl ; Cl] | N1-(3-(5-bromo-1- cyclohexyl-1H- indol-3-yl)-3- cyclohexylpropyl) propane- 1,3-diamine hydrochloride salt | 545.19 | 473.24 | 474.36 | I |
| 430 | 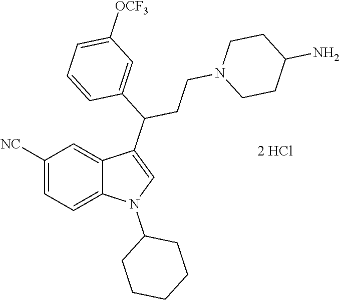 [SMILES: N#Cc1ccc2c(c1)c(C(CCN1CCC(N)CC1)c1cccc(OC(F)(F)F)c1)cn2C1CCCCC1] | 3-(3-(4-aminopiperidin-1- yl)-1-(3- (trifluoromethoxy)phenyl) propyl)-1-cyclohexyl-1H- indole-5-carbonitrile | | | 524.75 | I |
| 431 | 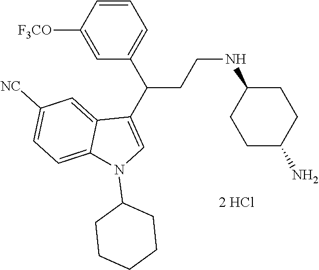 [SMILES: N#Cc1ccc2c(c1)c(C(CCN[C@H]1CC[C@H](N)CC1)c1cccc(OC(F)(F)F)c1)cn2C1CCCCC1] | 3-(3-(((1r,4r)-4- aminocyclohexyl) amino)-1-(3- (trifluoromethoxy) phenyl) propyl)-1- cyclohexyl-1H-indole- 5-carbonitrile dihydrochloride | | | 539 | I |
| 432 | 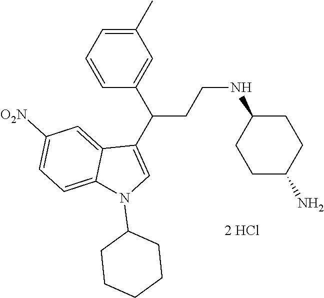 [SMILES: Cc1cccc(C(CCN[C@H]2CC[C@H](N)CC2)c2cn(C3CCCCC3)c3ccc([N+](=O)[O-])cc23)c1] | (1r,4r)-N1-(3-(1- cyclohexyl- 5-nitro-1H-indol- 3-yl)-3-(m- tolyl) propyl) cyclohexane- 1,4-diamine dihydrochloride | | | 488.79 | I |
| 433 | 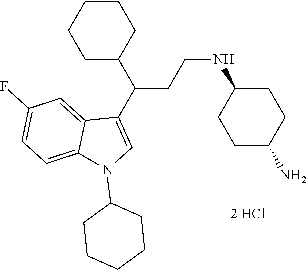 [SMILES: N[C@H]1CC[C@H](NCCC(c2cn(C3CCCCC3)c3ccc(F)cc23)C2CCCCC2)CC1] | (1r,4r)-N1-(3- cyclohexyl-3- (1-cyclohexyl-5- fluoro-1H- indol-3-yl) propyl) cyclohexane- 1,4-diamine dihydrochloride | | | 453.81 | I |
| 434 | 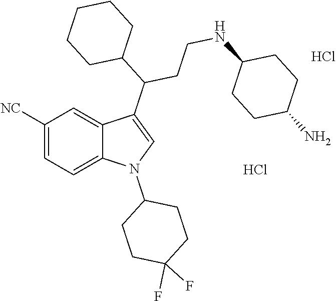 [SMILES: N#Cc1ccc2c(c1)c(C(CCN[C@H]1CC[C@H](N)CC1)C1CCCCC1)cn2C1CCC(F)(F)CC1 ; Cl ; Cl] | 3-(3-(((1r,4r)-4- aminocyclohexyl) amino)-1- cyclohexylpropyl)- 1-(4,4- difluorocyclohexyl)- 1H-indole-5- carbonitrile dihydrochloride | | | 496.81 | I |
| 435 | 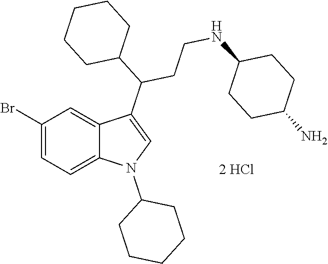 [SMILES: N[C@H]1CC[C@H](NCCC(c2cn(C3CCCCC3)c3ccc(Br)cc23)C2CCCCC2)CC1] | (1r,4r)-N1-(3-(5-bromo- 1-cyclohexyl-1H- indol-3-yl)-3 cyclohexylpropyl) cyclohexane-1,4-diamine dihydrochloride | | | 515.71 | I |
| 436 | 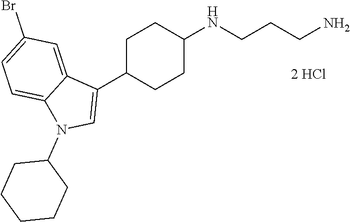 [SMILES: NCCCNC1CCC(c2cn(C3CCCCC3)c3ccc(Br)cc23)CC1] | N1-(4-(5-bromo-1- cyclohexyl-1H-indol-3-yl) cyclohexyl) propane-1,3- diamine dihydrochloride | | | 433.45 | IV |

TABLE XII
provides a summary of NMR data for the compounds synthesise

| Cmpd | Structure | NMR data |
|---|---|---|
| 7 | 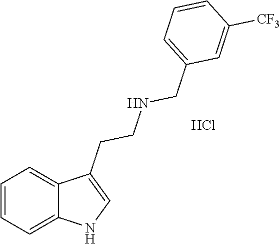 [SMILES: FC(F)(F)c1cccc(CNCCc2c[nH]c3ccccc23)c1 ; Cl] | ¹H NMR (400 MHz, DMSO-d₆): δ 10.96 (s, 1H), 9.27 (bs, 2H), 7.99 (s, 1H), 7.87 (d, J = 7.7 Hz, 1H), 7.80 (d, J = 8.1 Hz, 1H), 7.69 (t, J = 7.7 Hz, 1H), 7.56 (d, J = 7.9 Hz, 1H), 7.38-7.35 (m, 1H), 7.24 (d, J = 2.4 Hz, 1H), 7.13-7.07 (m, 1H), 7.04-6.98 (m, 1H), 4.32 (s, 2H), 3.24-3.16 (m, 2H), 3.15-3.08 (m, 2H); |
| 8 | 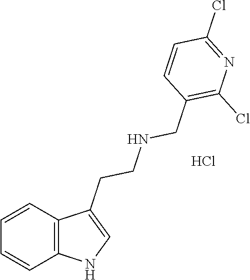 [SMILES: Clc1ccc(CNCCc2c[nH]c3ccccc23)c(Cl)n1 ; Cl] | ¹H NMR (400 MHz, DMSO-d₆): δ 10.96 (bs, 1H), 9.42 (bs, 2H), 8.25 (d, J = 7.6 Hz, 1H), 7.72 (d, J = 8.0 Hz, 1H), 7.58 (d, J = 7.6 Hz, 1H), 7.36 (d, J = 8.0 Hz, 1H), 7.25 (d, J = 2.0 Hz, 1H), 7.11-7.08 (m, 1H), 7.03-6.99 (m, 1H), 4.33 (s, 2H), 3.26 (t, J = 9.6 Hz, 2H), 3.11(t, J = 8.0 Hz, 2H); |
| 9 | 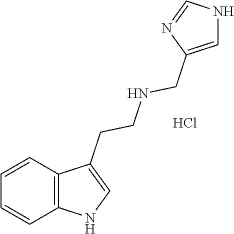 [SMILES: c1ccc2c(CCNCc3c[nH]cn3)c[nH]c2c1 ; Cl] | ¹H NMR (400 MHz, DMSO-d₆): δ 10.97 (bs, 1H), 9.91 (bs, 2H), 9.02 (s, 1H), 7.85-7.77 (m, 1H), 7.57 (d, J = 7.6 Hz, 1H), 7.37-7.24 (m, 2H), 7.15-6.62 (m, 3H), 4.32 (s, 2H), 3.21 (bs, 2H), 3.13 (bs, 2H); |
| 10 | 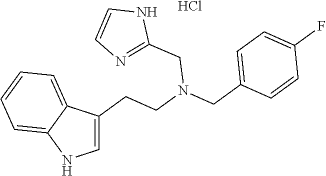 [SMILES: Cl ; Fc1ccc(CN(CCc2c[nH]c3ccccc23)Cc2ncc[nH]2)cc1] | ¹H NMR (400 MHz, DMSO-d₆): δ 10.88 (s, 1H), 7.70-7.57 (m, 4H), 7.32 (d, J = 8.7 Hz, 2H), 7.25-7.18 (m, 2H), 7.16- 7.12 (m, 1H), 7.08-7.02 (m, 1H), 6.95-6.89 (m, 1H), 4.66- 3.94 (m, 8H), 3.05 (s, 2H); |
| 11 | 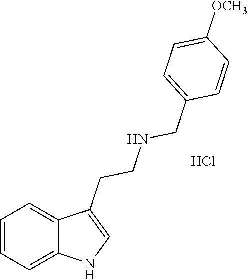 [SMILES: COc1ccc(CNCCc2c[nH]c3ccccc23)cc1 ; Cl] | ¹H NMR (400 MHz, DMSO-d₆): δ 10.95 (s, 1H), 9.02 (bs, 2H), 7.55 (d, J = 8.2 Hz, 1H), 7.47 (d, J = 8.6 Hz, 2H), 7.36 (d, J = 8.1 Hz, 1H), 7.21 (d, J = 2.4 Hz, 1H), 7.12-7.06 (m, 1H), 7.03-6.96 (m, 3H), 4.11 (s, 2H), 3.77 (s, 3H), 3.15-3.04 (m, 4H); |
| 12 | 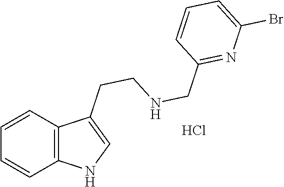 [SMILES: Brc1cccc(CNCCc2c[nH]c3ccccc23)n1 ; Cl] | ¹H NMR (400 MHz, DMSO-d₆): δ 10.96 (s, 1H), 9.23 (bs, 2H), 7.86 (t, J = 7.7 Hz, 1H), 7.71 (d, J = 7.9 Hz, 1H), 7.64- 7.54 (m, 2H), 7.36 (d, J = 8.1 Hz, 1H), 7.23 (d, J = 2.3 Hz, 1H), 7.10-6.97 (m, 2H), 4.38 (s, 2H), 3.29-3.21 (m, 2H), 3.18-3.08 (m, 2H); |
| 13 | 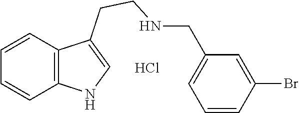 [SMILES: Brc1cccc(CNCCc2c[nH]c3ccccc23)c1 ; Cl] | ¹H NMR (400 MHz, Methanol-d₄): δ 7.70 (s, 1H), 7.62 (d, J = 7.2 Hz, 1H), 7.55 (d, J = 8.0 Hz, 1H), 7.44 (d, J = 7.2 Hz, 1H), 7.40-7.36 (m, 2H), 7.17 (s, 1H), 7.13 (t, J = 7.2 Hz, 1H), 7.05 (t, J = 7.2 Hz, 1H), 4.21 (s, 2H), 3.35 (t, J = 7.6 Hz, 2H), 3.18 (t, J = 7.6 Hz, 2H); |
| 14 | 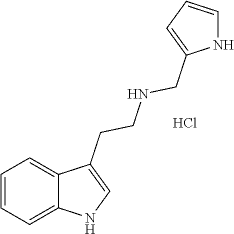 [SMILES: c1c[nH]c(CNCCc2c[nH]c3ccccc23)c1 ; Cl] | ¹H NMR (400 MHz, DMSO-d₆): δ 11.01-10.93 (m, 2H), 9.05 (bs, 2H), 7.54 (d, J = 8.0 Hz, 1H), 7.36 (d, J = 8.0 Hz, 1H), 7.21 (d, J = 2.4 Hz, 1H), 7.12-7.06 (m, 1H), 7.03-6.98 (m, 1H), 6.85-6.82 (m, 1H), 6.23-6.20 (m, 1H), 6.07-6.03 (m, 1H), 4.14 (s, 2H), 3.13-3.01 (m, 4H); |
| 15 | 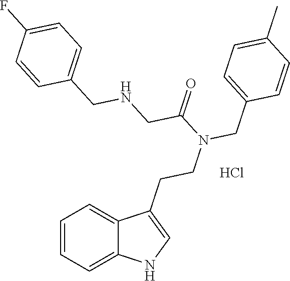 [SMILES: Cc1ccc(CN(CCc2c[nH]c3ccccc23)C(=O)CNCc2ccc(F)cc2)cc1 ; Cl] | ¹H NMR (400 MHz, DMSO-d₆): δ 9.35-9.16 (m, 2H), 7.56- 7.45 (m, 3H), 7.38-7.30 (m, 1H), 7.29-7.23 (m, 2H), 7.20- 7.08 (m, 6H), 7.07-6.93 (m, 2H), 4.61 (s, 1H), 4.47 (s, 1H), 4.17-4.12 (m, 1H), 4.04-4.00 (m, 1H), 3.90 (s, 2H), 3.57- 3.50 (m, 1H), 3.43-3.40 (m, 1H), 2.97-2.84 (m, 2H), 2.29 (d, J = 7.3 Hz, 3H); |
| 16 | 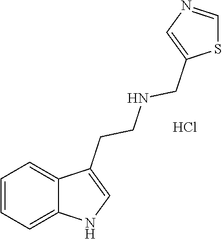 [SMILES: c1ccc2c(CCNCc3cncs3)c[nH]c2c1 ; Cl] | ¹H NMR (400 MHz, DMSO-d₆): δ 10.96 (s, 1H), 9.36 (bs, 2H), 9.23 (d, J = 1.9 Hz, 1H), 7.92 (d, J = 1.8 Hz, 1H), 7.55 (d, J = 7.8 Hz, 1H), 7.36 (d, J = 8.1 Hz, 1H), 7.22 (d, J = 2.4 Hz, 1H), 7.12-7.07 (m, 1H), 7.03-6.98 (m, 1H), 4.38 (t, J = 5.6 Hz, 2H), 3.23-3.17 (m, 2H), 3.13-3.07 (m, 2H); |
| 17 | 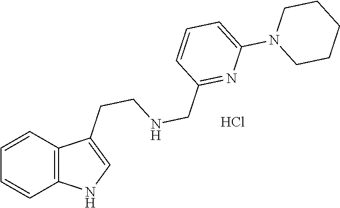 [SMILES: c1cc(CNCCc2c[nH]c3ccccc23)nc(N2CCCCC2)c1 ; Cl] | ¹H NMR (400 MHz, DMSO-d₆): δ 10.96 (s, 1H), 9.06 (br s, 2H), 7.61-7.48 (m, 2H), 7.36 (d, J = 7.9 Hz, 1H), 7.22 (d, J = 1.7 Hz, 1H), 7.09 (t, J = 7.5 Hz, 1H), 7.04-6.95 (m, 1H), 6.81 (d, J = 8.7 Hz, 1H), 6.66 (d, J = 7.2 Hz, 1H), 4.22-4.16 (m, 4H), 3.54 (t, J = 4.9 Hz, 4H), 3.33-3.19 (m, 2H), 3.18- 3.05 (m, 2H), 1.67-1.47 (m, 6H); |
| 18 | 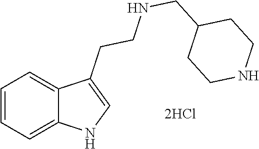 [SMILES: c1ccc2c(CCNCC3CCNCC3)c[nH]c2c1 ; "2HCl"] | ¹H NMR (400 MHz, DMSO-d₆): δ 10.98 (bs, 1H), 9.22 (s, 2H), 9.12-9.01 (m, 1H), 8.97-8.82 (m, 1H), 7.62 (d, J = 7.8 Hz, 1H), 7.36 (d, J = 8.0 Hz, 1H), 7.22 (d, J = 2.3 Hz, 1H), 7.13-7.06 (m, 1H), 7.04-6.97 (m, 1H), 3.26 (d, J = 12.7 Hz, 2H), 3.14 (s, 4H), 2.94-2.80 (m, 4H), 2.11-1.93 (m, 3H), 1.51-1.39 (m, 2H); |
| 19 | 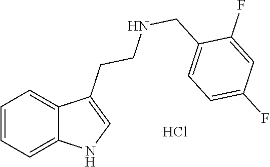 [SMILES: Fc1ccc(CNCCc2c[nH]c3ccccc23)c(F)c1 ; Cl] | ¹H NMR (400 MHz, Methanol-d₄): δ 7.57-7.51 (m, 2H), 7.37 (d, J = 8.0 Hz, 1H), 7.18 (s, 1H), 7.16-7.09 (m, 2H), 7.08-7.03 (m, 2H), 4.29 (s, 2H), 3.37 (t, J = 8.0 Hz, 2H), 3.19 (t, J = 8.0 Hz, 2H); |
| 20 | 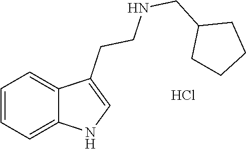 [SMILES: c1ccc2c(CCNCC3CCCC3)c[nH]c2c1 ; Cl] | ¹H NMR (400 MHz, Methanol-d₄): δ 7.58 (d, J = 7.6 Hz, 1H), 7.37 (d, J = 7.6 Hz, 1H), 7.18 (s, 1H), 7.13 (t, J = 7.6 Hz, 1H), 7.05 (t, J = 7.6 Hz, 1H), 3.21-3.14 (m, 2H), 3.01 (t, J = 7.6 Hz, 1H), 2.18 (m, 1H), 1.94-1.83 (m, 2H), 1.75-1.56 (m, 4H), 1.32-1.22 (m, 2H); |
| 21 | 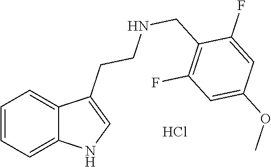 [SMILES: COc1cc(F)c(CNCCc2c[nH]c3ccccc23)c(F)c1 ; Cl] | ¹H NMR (400 MHz, Methanol-d₄): δ 7.55 (d, J = 8.0 Hz, 1H), 7.37 (d, J = 8.0 Hz, 1H), 7.18 (s, 1H), 7.13 (t, J = 7.4 Hz, 1H), 7.04 (t, J = 7.4 Hz, 1H), 6.72 (d, J = 9.6 Hz, 2H), 4.28 (s, 2H), 3.84 (s, 3H), 3.35 (t, J = 8.0 Hz, 2H), 3.19 (t, J = 8.0 Hz, 2H); |
| 22 | 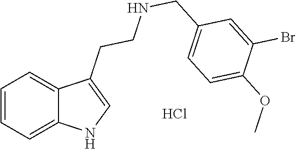 [SMILES: COc1ccc(CNCCc2c[nH]c3ccccc23)cc1Br ; Cl] | ¹H NMR (400 MHz, Methanol-d₄): δ 7.68 (s, 1H), 7.53 (d, J = 8.0 Hz, 1H), 7.40 (m, 1H), 7.36 (s, 1H), 7.16 (s, 1H), 7.11 (m, 1H), 7.09 (d, J = 8.8 Hz, 1H), 7.04 (d, J = 7.6 Hz, 1H), 4.13 (s, 2H), 3.90 (s, 3H), 3.36-3.32 (m, 2H), 3.17-3.10 (m, 2H); |
| 23 | 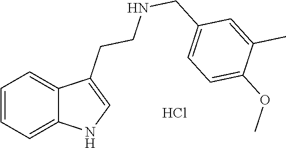 [SMILES: COc1ccc(CNCCc2c[nH]c3ccccc23)cc1C ; Cl] | ¹H NMR (400 MHz, Methanol-d₄): δ 7.52 (d, J = 8.0 Hz, 1H), 7.37 (d, J = 8.0 Hz, 1H), 7.25 (dd, J = 8.4, 2.0 Hz, 1H), 7.21 (s, 1H), 7.16 (s, 1H), 7.13 (t, J = 7.2 Hz, 1H), 7.03 (t, J = 7.2 Hz, 1H), 6.95 (d, J = 8.4 Hz, 1H), 4.10 (s, 2H), 3.58 (s, 3H), 3.30-3.28 (m, 2H), 3.16 (t, J = 7.6 Hz, 2H), 2.82 (s, 3H); |
| 24 | 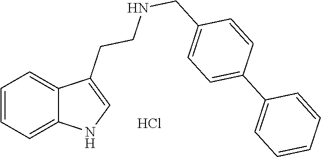 [SMILES: c1ccc(-c2ccc(CNCCc3c[nH]c4ccccc34)cc2)cc1 ; Cl] | ¹H NMR (400 MHz, DMSO-d₆): δ 10.96 (s, 1H), 9.29 (s, 2H), 7.77-7.72 (m, 2H), 7.71-7.68 (m, 2H), 7.67-7.63 (m, 2H), 7.57 (d, J = 7.9 Hz, 1H), 7.51-7.46 (m, 2H), 7.42-7.34 (m, 2H), 7.23 (d, J = 2.4 Hz, 1H), 7.12-7.07 (m, 1H), 7.03- 6.98 (m, 1H), 4.24 (s, 2H), 3.23-3.10 (m, 4H); |
| 25 | 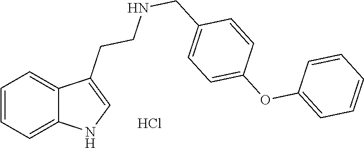 [SMILES: c1ccc(Oc2ccc(CNCCc3c[nH]c4ccccc34)cc2)cc1 ; Cl] | ¹H NMR (400 MHz, DMSO-d₆): δ 10.96 (s, 1H), 9.18 (s, 2H), 7.60-7.53 (m, 3H), 7.45-7.34 (m, 3H), 7.22 (d, J = 2.4 Hz, 1H), 7.21-7.15 (m, 1H), 7.13-6.98 (m, 6H), 4.17 (s, 2H), 3.20-3.07 (m, 4H); |
| 26 | 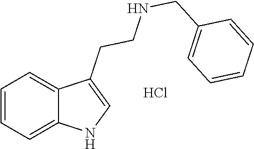 [SMILES: c1ccc(CNCCc2c[nH]c3ccccc23)cc1 ; Cl] | ¹H NMR (400 MHz, DMSO-d₆): δ 10.95 (s, 1H), 9.25 (s, 2H), 7.59-7.53 (m, 3H), 7.48-7.41 (m, 3H), 7.38-7.34 (m, 1H), 7.22 (d, J = 2.4 Hz, 1H), 7.12-7.06 (m, 1H), 7.03-6.98 (m, 1H), 4.19 (s, 2H), 3.18-3.08 (m, 4H); |
| 27 | 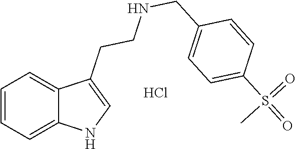 [SMILES: CS(=O)(=O)c1ccc(CNCCc2c[nH]c3ccccc23)cc1 ; Cl] | ¹H NMR (400 MHz, DMSO-d₆): δ 10.96 (s, 1H), 9.46 (s, 2H), 8.00 (d, J = 8.2 Hz, 2H), 7.87-7.82 (m, 2H), 7.57 (d, J = 7.7 Hz, 1H), 7.37 (d, J = 8.0 Hz, 1H), 7.23 (d, J = 2.3 Hz, 1H), 7.13-7.06 (m, 1H), 7.04-6.98 (m, 1H), 4.32 (s, 2H), 3.24 (s, 3H), 3.22-3.10 (m, 4H); |
| 28 | 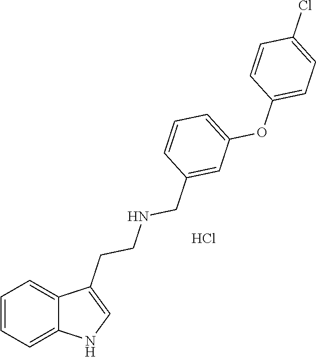 [SMILES: Clc1ccc(Oc2cccc(CNCCc3c[nH]c4ccccc34)c2)cc1 ; Cl] | ¹H NMR (400 MHz, DMSO-d₆): δ 10.93 (s, 1H), 8.69 (br s, 2H), 7.53 (d, J = 7.8 Hz, 1H), 7.48-7.39 (m, 3H), 7.35 (d, J = 8.0 Hz, 1H), 7.31 (d, J = 7.5 Hz, 1H), 7.22 (s, 1H), 7.19 (d, J = 2.3 Hz, 1H), 7.12-7.01 (m, 4H), 7.01-6.95 (m, 1H), 4.12 (s, 2H), 3.15-3.00 (m, 4H); |
| 29 | 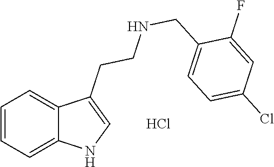 [SMILES: Fc1cc(Cl)ccc1CNCCc1c[nH]c2ccccc12 ; Cl] | ¹H NMR (400 MHz, DMSO-d₆): δ 10.98 (s, 1H), 9.56 (s, 2H), 7.78 (t, J = 8.3 Hz, 1H), 7.61-7.52 (m, 2H), 7.43-7.34 (m, 2H), 7.23 (d, J = 2.4 Hz, 1H), 7.12-7.06 (m, 1H), 7.04- 6.98 (m, 1H), 4.23 (s, 2H), 3.24-3.11 (m, 4H); |
| 31 | 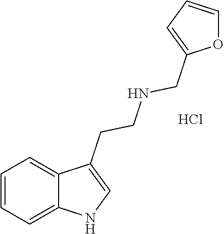 [SMILES: c1coc(CNCCc2c[nH]c3ccccc23)c1 ; Cl] | ¹H NMR (400 MHz, DMSO-d₆): δ 10.98 (s, 1H), 9.43 (s, 2H), 7.79-7.76 (m, 1H), 7.56 (d, J = 7.9 Hz, 1H), 7.36 (d, J = 8.0 Hz, 1H), 7.21 (d, J = 2.4 Hz, 1H), 7.12-7.07 (m, 1H), 7.03-6.97 (m, 1H), 6.66 (d, J = 3.1 Hz, 1H), 6.53 (dd, J = 3.2, 1.9 Hz, 1H), 4.27 (s, 2H), 3.18-3.05 (m, 4H); |
| 32 | 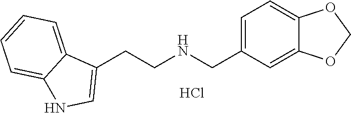 [SMILES: c1ccc2c(CCNCc3ccc4c(c3)OCO4)c[nH]c2c1 ; Cl] | ¹H NMR (400 MHz, DMSO-d₆): δ 10.95 (s, 1H), 9.21 (s, 2H), 7.56 (d, J = 7.9 Hz, 1H), 7.36 (d, J = 8.0 Hz, 1H), 7.21 (d, J = 2.4 Hz, 1H), 7.18 (d, J = 1.6 Hz, 1H), 7.12-7.07 (m, 1H), 7.04-6.98 (m, 2H), 6.96 (d, J = 8.0 Hz, 1H), 6.05 (s, 2H), 4.09 (s, 2H), 3.10 (s, 4H); |
| 34 | 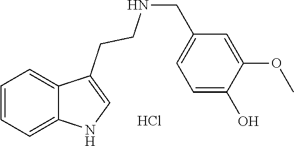 [SMILES: COc1cc(CNCCc2c[nH]c3ccccc23)ccc1O ; Cl] | ¹H NMR (400 MHz, Methanol-d₄): δ 7.53 (d, J = 8.0 Hz, 1H), 7.37 (d, J = 8.0 Hz, 1H), 7.16 (s, 1H), 7.13 (t, J = 7.6 Hz, 1H), 7.03 (t, J = 7.6 Hz, 1H), 7.00 (s, 1H), 6.89 (dd, J = 8.4, 2.0 Hz, 1H), 6.84 (d, J = 8.4 Hz, 1H), 4.11 (s, 2H), 3.58 (s, 3H), 3.36-3.33 (m, 2H), 3.20-3.13 (m, 2H); |
| 35 | 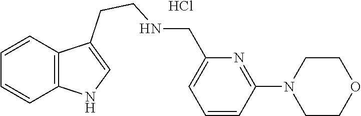 [SMILES: c1cc(CNCCc2c[nH]c3ccccc23)nc(N2CCOCC2)c1 ; Cl] | ¹H NMR (400 MHz, DMSO-d₆): δ 10.97 (s, 1H), 9.12 (s, 1H), 7.64-7.52 (m, 2H), 7.36 (d, J = 8.1 Hz, 1H), 7.25-7.21 (m, 1H), 7.12-7.06 (m, 1H), 7.02-6.97 (m, 1H), 6.81 (d, J = 8.5 Hz, 1H), 6.76 (d, J = 7.1 Hz, 1H), 4.21 (t, J = 5.7 Hz, 2H), 3.69 (t, J = 4.8 Hz, 4H), 3.47 (t, J = 4.8 Hz, 4H), 3.29- 3.18 (m, 2H), 3.17-3.09 (m, 2H); |
| 36 | 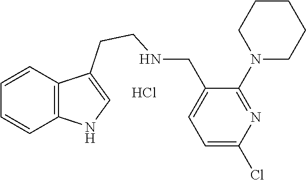 [SMILES: Clc1ccc(CNCCc2c[nH]c3ccccc23)c(N2CCCCC2)n1 ; Cl] | ¹H NMR (400 MHz, DMSO-d₆): δ 10.97 (s, 1H), 9.46 (bs, 2H), 8.14 (d, J = 8.0 Hz, 1H), 7.55 (d, J = 7.9 Hz, 1H), 7.36 (d, J = 8.0 Hz, 1H), 7.27-7.16 (m, 2H), 7.13-7.04 (m, 1H), 7.04-6.95 (m, 1H), 4.17 (t, J = 5.8 Hz, 2H), 3.31-3.18 (m, 2H), 3.18-3.08 (m, 2H), 3.04-2.88 (m, 4H), 1.66-1.55 (m, 4H), 1.55-1.46 (m, 2H); |
| 37 | 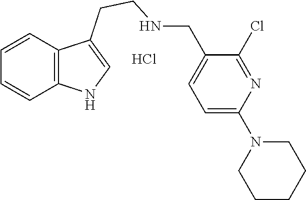 [SMILES: Clc1nc(N2CCCCC2)ccc1CNCCc1c[nH]c2ccccc12 ; Cl] | ¹H NMR (400 MHz, DMSO-d₆): δ 10.96 (s, 1H), 9.13 (bs, 2H), 7.79 (d, J = 8.7 Hz, 1H), 7.56 (d, J = 7.8 Hz, 1H), 7.36 (d, J = 8.1 Hz, 1H), 7.23 (d, J = 2.3 Hz, 1H), 7.12-7.06 (m, 1H), 7.04-6.97 (m, 1H), 6.86 (d, J = 8.7 Hz, 1H), 4.14 (t, J = 5.6 Hz, 2H), 3.59-3.47 (m, 4H), 3.27-3.15 (m, 2H), 3.15- 3.05 (m, 2H), 1.68-1.56 (m, 2H), 1.56-1.44 (m, 4H); |
| 38 | 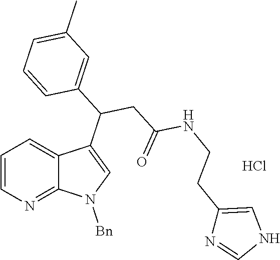 [SMILES: Cc1cccc(C(CC(=O)NCCc2c[nH]cn2)c2cn(Cc3ccccc3)c3ncccc23)c1 ; Cl] | ¹H NMR (400 MHz, DMSO-d₆): δ 14.27 (s, 1H), 14.05 (s, 1H), 8.96 (s, 1H), 8.49 (dd, J = 1.4 Hz, 1H), 8.05 (t, J = 5.8 Hz, 1H), 7.72 (dd, J = 1.52 Hz, 1H), 7.54 (s, 1H), 7.30- 7.18 (m, 6H), 7.13-6.93 (m, 5H), 5.44 (s, 2H), 4.61 (t, J = 8.0 Hz, 1H), 3.26-3.17 (m, 2H), 2.85 (dd, J = 7.8 Hz, 1H), 2.71 (dd, J = 7.8 Hz, 1H), 2.57 (t, J = 6.8 Hz, 2H), 2.21 (s, 3H); |
| 39 | 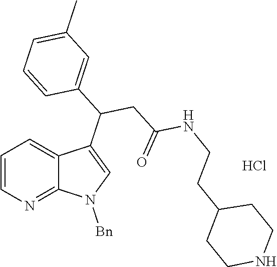 [SMILES: Cc1cccc(C(CC(=O)NCCC2CCNCC2)c2cn(Cc3ccccc3)c3ncccc23)c1 ; Cl] | ¹H NMR (400 MHz, DMSO-d₆): δ 8.80 (bs, 1H), 8.53 (bs, 1H), 8.21 (d, J = 4.2 Hz, 1H), 7.89-7.82 (m, 2H), 7.6 (s, 1H), 7.32-7.31 (m, 5H), 7.11-7.03 (m, 4H), 6.94 (d, J = 6.9 Hz, 1H), 5.47 (s, 2H), 4.63 (t, J = 7.8 Hz, 1H), 3.51- 3.48 (m, 1H), 3.14 (d, J = 8.0 Hz, 2H), 3.02-2.72 (m, 5H), 2.23 (s, 3H), 1.59 (t, J = 8.0 Hz, 2H), 1.23-1.12 (m, 5H); |
| 40 | 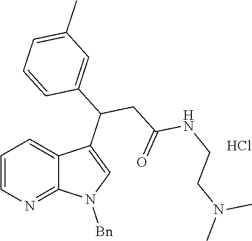 [SMILES: Cc1cccc(C(CC(=O)NCCN(C)C)c2cn(Cc3ccccc3)c3ncccc23)c1 ; Cl] | ¹H NMR (400 MHz, DMSO-d₆): δ 10.08 (bs, 1H), 8.26 (t, J = 5.64 Hz, 1H), 8.19 (dd, J = 4.8, 1.2 Hz, 1H), 7.76 (dd, J = 8.0, 2.4 Hz, 1H), 7.61 (s, 1H), 7.32-7.29 (m, 2H), 7.29- 7.20 (m, 3H), 7.14-7.03 (m, 3H), 7.02-6.95 (m, 2H), 5.46 (s, 2H), 4.65 (d, J = 8.0 Hz, 1H), 3.32-3.23 (m, 2H), 2.98- 2.90 (m, 3H), 2.83-2.78 (m, 1H), 2.63 (s, 6H), 2.23 (s, 3H); |
| 41 | 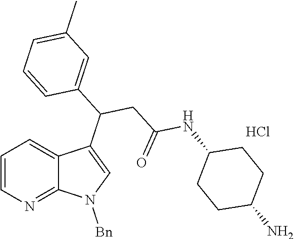 [SMILES: Cc1cccc(C(CC(=O)N[C@H]2CC[C@@H](N)CC2)c2cn(Cc3ccccc3)c3ncccc23)c1 ; Cl] | ¹H NMR (400 MHz, DMSO-d6): δ 8.19 (d, J = 3.2 Hz, 1H), 7.90 (bs, 3H), 7.72 (t, J = 7.2 Hz, 2H), 7.57 (s, 1H), 7.31- 7.24 (m, 3H), 7.19-7.06 (m, 5H), 7.101-6.93 (m, 2H), 5.51 (q, J = 15.2 Hz, 2H), 4.63 (t, J = 8.0 Hz, 1H), 3.51- 3.39 (m, 3H), 2.97-2.92 (m, 1H), 2.80-2.75 (m, 1H), 2.22 (s. 3H), 1.61-1.48 (m, 5H), 1.39-1.37 (m, 2H); |
| 43 | 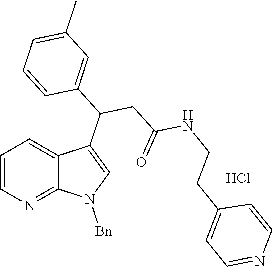 [SMILES: Cc1cccc(C(CC(=O)NCCc2ccncc2)c2cn(Cc3ccccc3)c3ncccc23)c1 ; Cl] | ¹H NMR (400 MHz, DMSO-d₆): δ 8.64 (d, J = 6.8 Hz, 2H), 8.20 (dd, J = 4.8, 1.6 Hz, 1H), 8.06 (t, J = 5.8 Hz, 1H), 7.73 (dd, J = 8.0, 1.6 Hz, 1H), 7.67 (d, J = 6.4 Hz, 2H), 7.54 (s, 1H), 7.30-7.19 (m, 5H), 6.93-7.11 (m, 5H), 5.44 (s, 2H), 4.58 (t, J = 8.0 Hz, 1H), 3.39-3.24 (m, 2H), 2.89-2.78 (m, 3H), 2.69-2.66 (m, 1H), 2.21 (s, 3H) ); |
| 44 | 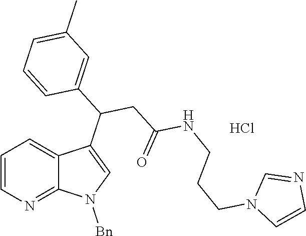 [SMILES: Cc1cccc(C(CC(=O)NCCCn2ccnc2)c2cn(Cc3ccccc3)c3ncccc23)c1 ; Cl] | ¹H NMR (400 MHz, DMSO-d₆): δ 14.2 (bs, 1H), 8.98 (s, 1H), 8.19 (dd, J = 8.0, 1.6 Hz, 1H), 8.09 (t, J = 6.0 Hz, 1H), 7.77 (dd, J = 8.0, 1.6 Hz, 1H), 7.66 (s, 1H), 7.61-7.59 (m, 2H), 7.29-7.24 (m, 5H), 7.23-7.11 (m, 3H), 7.02-6.99 (m, 1H), 6.94-6.92 (m, 1H), 5.4 (q, J = 15.2 Hz, 2H), 4.64 (t, J = 8.0 Hz, 1H), 3.82 (t, J = 6.8 Hz, 2H), 2.97-2.76 (m, 4H), 2.21(s, 3H), 1.70 (t, J = 6.8 Hz, 2H); |
| 45 | 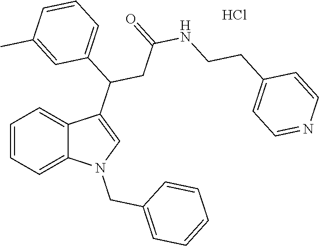 [SMILES: Cc1cccc(C(CC(=O)NCCc2ccncc2)c2cn(Cc3ccccc3)c3ccccc23)c1 ; Cl] | ¹H NMR (400 MHz, DMSO-d₆): δ 8.51-8.48 (m, 2H), 7.98 (t, J = 5.74 Hz, 1H), 7.44-7.42 (m, 2H), 7.41 (bs, 1H), 7.36 (d, J = 8.16 Hz, 1H), 7.33-7.25 (m, 3H), 7.24-7.08 (m, 5H), 7.06-7.02 (m, 3H), 6.95-6.88 (m, 2H), 5.36 (bs, 2H), 4.61 (t, J = 8.0 Hz, 1H), 3.23-3.18 (m, 2H), 2.89-2.82 (m, 1H), 2.77- 2.67 (m, 3H), 2.21 (s, 3H); |
| 51 | 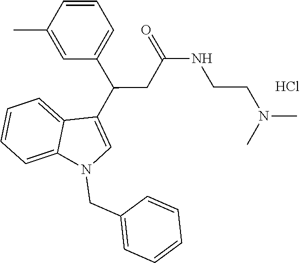 [SMILES: Cc1cccc(C(CC(=O)NCCN(C)C)c2cn(Cc3ccccc3)c3ccccc23)c1 ; Cl] | ¹H NMR (400 MHz, DMSO-d₆): δ □ 9.46 (bs, 1H), 8.17 (t, J = 5.75 Hz, 1H), 7.44 (bs, 1H), 7.36-7.28 (m, 4H), 7.26-7.23 (m, 1H), 7.18-7.15 (m, 2H), 7.11-7.07 (m, 3H), 7.04-7.01 (m, 1H), 6.96-6.90 (m, 2H), 5.38 (bs, 2H), 4.65 (t, J = 8.0 Hz, 1H), 2.97-2.90 (m, 4H), 2.80-2.78 (m, 2H), 2.66 (bs, 6H), 2.23 (bs, 3H); |
| 52 | 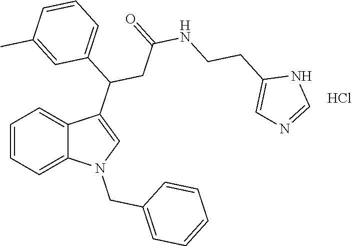 [SMILES: Cc1cccc(C(CC(=O)NCCc2cnc[nH]2)c2cn(Cc3ccccc3)c3ccccc23)c1 ; Cl] | ¹H NMR (400 MHz, DMSO-d₆): δ 14.26-13.89 (bs, 2H), 8.94 (d, J = 1.28 Hz, 1H), 8.03 (t, J = 5.83 Hz, 1H), 7.41 (bs, 1H), 7.36 (d, J = 8.4 Hz, 1H), 7.33-7.26 (m, 3H), 7.24- 7.19 (m, 2H), 7.16-7.13 (m, 2H), 7.13-7.08 (m, 1H), 7.08- 7.00 (m, 3H), 6.94-6.87 (m, 2H), 5.37 (bs, 2H), 4.62 (t, J = 8.0 Hz, 1H), 3.27-3.16 (m, 2H), 2.87 (dd, J = 14.2, 8.0 Hz, 1H), 2.74-2.72 (m, 1H), 2.61 (t, J = 7.00 Hz, 2H), 2.22-2.20 (s, 3H); |
| 53 | 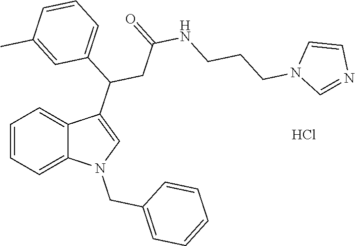 [SMILES: Cc1cccc(C(CC(=O)NCCCn2ccnc2)c2cn(Cc3ccccc3)c3ccccc23)c1 ; Cl] | ¹H NMR (400 MHz, DMSO-d₆): δ 14.43 (bs, 1H), 8.95 (bs, 1H), 8.13 (t, J = 5.74 Hz, 1H), 7.65 (t, J = 1.68, 1H), 7.59 (t, J = 1.68, 1H), 7.48 (bs, 1H), 7.39-7.33 (m, 2H), 7.29-7.20 (m, 3H), 7.18-7.14 (m, 2H), 7.13-7.10 (m, 3H), 7.05-7.00 (m, 1H), 6.93-6.87 (m, 2H), 5.37 (bs, 2H), 4.66 (t, J = 7.75 Hz, 1H), 3.83 (t, J = 6.75 Hz, 2H), 2.99-2.85 (m, 3H), 2.78 (dd, J = , 13.97, 7.86, Hz, 1H), 2.21 (s, 3H), 1.78-1.70 (s, 2H); |
| 54 | 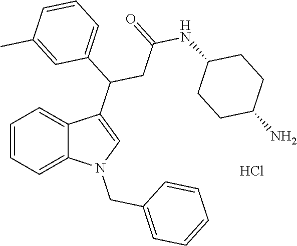 [SMILES: Cc1cccc(C(CC(=O)N[C@H]2CC[C@@H](N)CC2)c2cn(Cc3ccccc3)c3ccccc23)c1 ; Cl] | ¹H NMR (400 MHz, DMSO-d₆): δ 7.80 (bs, 2H), 7.75-7.73 (m, 2H), 7.42 (bs, 1H), 7.36-7.23 (m, 5H), 7.18-7.14 (m, 2H), 7.11-7.01 (m, 4H), 6.93 (d, J = 7.28 Hz, 1H), 6.88 (t, J = 7.28 Hz, 1H), 5.37 (bs, 2H), 4.63 (t, J = 8.0 Hz, 1H), 2.94- 2.82 (m, 2H), 2.67 (dd, J = 14.04, 7.62 Hz, 1H), 2.22 (bs, 3H), 1.88-1.80 (m, 2H), 1.65-1.53 (m, 3H), 1.36-1.18 (m, 2H), 1.08-1.03 (m, 2H); |
| 55 | 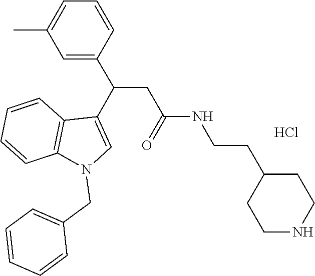 [SMILES: Cc1cccc(C(CC(=O)NCCC2CCNCC2)c2cn(Cc3ccccc3)c3ccccc23)c1 ; Cl] | ¹H NMR (400 MHz, DMSO-d₆): δ 8.49 (bs, 1H), 8.21 (bs, 1H), 7.81 (t, J = 5.74 Hz, 1H), 7.43 (bs, 1H), 7.36 (d, J = 8.61 Hz, 2H), 7.32-7.23 (m, 3H), 7.19-7.15 (m, 2H), 7.13- 7.08 (m, 3H), 7.03 (t, J = 7.76 Hz, 1H), 6.95-6.88 (m, 2H), 5.37 (bs, 2H), 4.64 (t, J = 7.98 Hz, 1H), 3.19-2.98 (m, 4H), 2.95-2.83 (m, 2H), 2.74 (dd, J = 14.0, 8.10 Hz, 1H), 2.61- 2.55 (m, 1H), 2.23 (bs, 3H), 1.67-1.55 (m, 2H), 1.20-1.08 (m, 5H); |
| 58 | 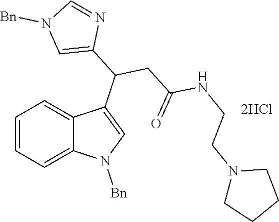 [SMILES: O=C(CC(c1cn(Cc2ccccc2)cn1)c1cn(Cc2ccccc2)c2ccccc12)NCCN1CCCC1 ; "2HCl"] | ¹H NMR (400 MHz, DMSO-d₆): δ 11.1 (s, 1H), 10.70 (bs, 1H), 9.28 (s, 1H), 7.99 (s, 1H), 8.40 (t, J = 5.6 Hz, 1H), 7.43- 7.32 (m, 10H), 7.53-7.47 (m, 1H), 7.08-7.05 (m, 1H), 6.85 (t, J = 8.0 Hz, 1H), 5.41-5.26 (m, 4H), 4.80 (t, J = 7.2 Hz, 1H), 3.45-3.31 (m, 2H), 3.08-3.01 (m, 2H), 2.93- 3.0 (m, 4H), 2.92-2.90 (m, 3H), 1.95-1.90 (m, 1H), 1.89- 1.86 (m, 2H); |
| 59 | 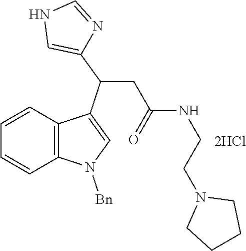 [SMILES: O=C(CC(c1c[nH]cn1)c1cn(Cc2ccccc2)c2ccccc12)NCCN1CCCC1 ; "2HCl"] | ¹H NMR (400 MHz, DMSO-d₆): δ 14.48 (bs, 1H), 11.05 (s, 1H), 10.47 (bs, 1H), 9.07 (bs, 1H), 8.36 (s, 1H), 7.63 (bs, 1H), 7.41-7.34 (m, 7H), 7.27 (s, 1H), 7.09-6.93 (m, 2H), 5.34 (s, 2H), 4.80 (t, J = 7.6 Hz, 1H), 3.36-3.31 (m, 2H), 3.04-3.02 (m, 2H), 2.98-2.80 (m, 6H), 1.85 (bs, 4H); |
| 60 | 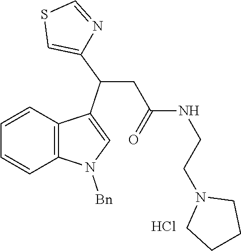 [SMILES: O=C(CC(c1cscn1)c1cn(Cc2ccccc2)c2ccccc12)NCCN1CCCC1 ; Cl] | ¹H NMR (400 MHz, DMSO-d₆): δ 10.24 (bs, 1H), 9.0 (s, 1H), 8.27 (t, J = 5.6 Hz, 1H), 7.50 (d, J = 7.8 Hz, 1H), 7.41 (s, 1H), 7.38-7.28 (m, 4H), 7.25-7.23 (m, 1H), 7.19-7.16 (m, 2H), 7.0 (dt, J = 8.0, 1.0 Hz, 1H), 6.93 (dt, J = 7.2, 1.0 Hz, 1H), 5.36 (s, 2H), 4.93 (t, J = 7.6 Hz, 1H), 3.42-3.38 (m, 2H), 3.28 (q, J = 5.6 Hz, 2H), 3.07-2.93 (m, 4H), 2.75- 2.71 (m, 2H), 1.84-1.79 (m, 2H), 1.76-1.74 (m, 2H); |
| 64 | 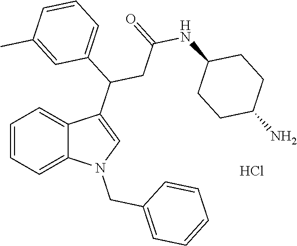 [SMILES: Cc1cccc(C(CC(=O)N[C@H]2CC[C@H](N)CC2)c2cn(Cc3ccccc3)c3ccccc23)c1 ; Cl] | ¹H NMR (400 MHz, DMSO-d₆): δ 7.78 (bs, 3H), 7.71 (d, J = 6.06 Hz, 1H), 7.43 (bs, 1H), 7.36-7.23 (m, 5H), 7.16-7.07 (m, 5H), 7.04-7.00 (m, 1H), 6.94-6.86 (m, 2H), 5.38 (bs, 2H), 4.64 (t, J = 8.0 Hz, 1H), 3.63-3.57 (m, 1H), 3.08-2.87 (m, 3H), 2.80-2.74 (m, 1H), 2.22 (bs, 3H), 1.64-1.57 (m, 3H), 1.55-1.49 (m, 2H), 1.47-1.40 (m, 2H); |
| 66 | 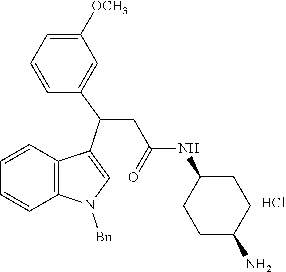 [SMILES: COc1cccc(C(CC(=O)N[C@H]2CC[C@@H](N)CC2)c2cn(Cc3ccccc3)c3ccccc23)c1 ; Cl] | ¹H NMR (400 MHz, DMSO-d₆): 7.83 (bs, 3H), 7.72 (d, J = 6.24 Hz, 1H), 7.45 (s, 1H), 7.36-7.23 (m, 5H), 7.16-7.12 (m, 3H), 7.0 (d, J = 10.4 Hz, 1H), 6.86, (dd, J = 7.2 Hz, 1H), 6.78-6.65 (m, 2H), 6.61-6.56 (m, 1H), 5.39 (s, 2H), 4.64 (t, J = 7.6 Hz, 1H), 3.68 (s, 3H), 3.58 (bs, 1H), 3.02 (bs, 1H), 2.91 (d, J = 8.0 Hz, 1H), 2.76 (d, J = 8.0 Hz, 1H), 1.62-1.43 (m, 8H); |
| 67 | 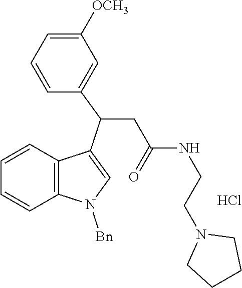 [SMILES: COc1cccc(C(CC(=O)NCCN2CCCC2)c2cn(Cc3ccccc3)c3ccccc23)c1 ; Cl] | ¹H NMR (400 MHz, DMSO-d₆): 10.19 (bs, 1H), 8.27 (t, J = 5.6 Hz, 1H), 7.5 (s, 1H), 7.38-7.14 (m, 8H), 7.05-7.01 (m, 1H), 6.95-6.84 (m, 3H), 6.72-6.70 (m, 1H), 5.38 (s, 2H), 4.67 (t, J = 7.6 Hz, 1H), 3.68 (s, 3H), 3.32-3.24 (m, 3H), 3.05-2.92 (m, 3H), 2.85-2.66 (m, 4H), 1.87-1.72 (m, 4H) |
| 68 | 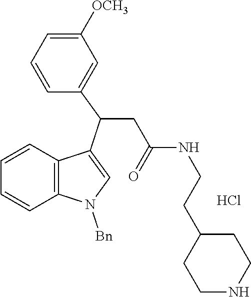 [SMILES: COc1cccc(C(CC(=O)NCCC2CCNCC2)c2cn(Cc3ccccc3)c3ccccc23)c1 ; Cl] | ¹H NMR (400 MHz, DMSO-d₆): δ 8.57 (bs, 1H), 8.30 (bs, 1H), 7.82 (t, J = 5.6 Hz, 1H), 7.49-7.30 (m, 3H), 7.29- 7.18 (m, 6H), 7.13 (t, J = 6.4 Hz, 1H), 7.05-6.88 (m, 3H), 6.68 (dd, J = 5.6, 1.6 Hz, 1H), 5.37 (s, 2H), 4.64 (t, J = 8.0 Hz, 1H), 3.68 (s, 3H), 3.57-3.46 (m, 2H), 3.16-3.01 (m, 4H), 2.90-2.56 (m, 3H), 1.65-1.57 (m, 2H), 1.16-1.10 (m, 4H); |
| 69 | 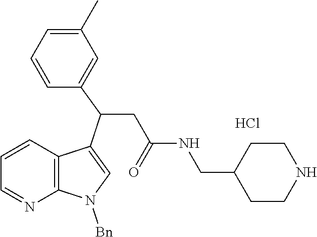 [SMILES: Cc1cccc(C(CC(=O)NCC2CCNCC2)c2cn(Cc3ccccc3)c3ncccc23)c1 ; Cl] | ¹H NMR (400 MHz, DMSO-d₆): δ 8.68 (bs, 1H), 8.39 (bs, 1H), 8.19 (d, J = 7.8 Hz, 1H), 7.92 (t, J = 5.6 Hz, 1H), 7.77 (d, J = 7.6 Hz, 1H), 7.56 (s, 1H), 7.32-7.20 (m, 5H), 7.15- 7.08 (m, 3H), 7.03-6.94 (m, 2H), 5.45 (s, 2H), 4.61 (t, J = 7.6 Hz, 1H), 3.77-3.66 (m, 1H), 3.50-3.46 (m, 1H), 3.06 (bs, 2H), 2.91-2.80 (m, 2H), 2.78-2.68 (m, 2H), 2.23 (s, 3H), 1.37-1.34 (m, 3H), 1.07-1.04 (m, 2H); |
| 70 | 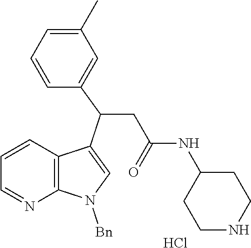 [SMILES: Cc1cccc(C(CC(=O)NC2CCNCC2)c2cn(Cc3ccccc3)c3ncccc23)c1 ; Cl] | ¹H NMR (400 MHz, DMSO-d₆): bs, 2H), 8.19 (d, J = 8.0 Hz, 1H), 8.01 (d, J = 7.6 Hz, 1H), 7.74 (d, J = 6.8 Hz, 1H), 7.55 (s, 1H), 7.32-7.12 (m, 5H), 6.94-7.10 (m, 5H), 5.41 (q, J = 15.6 Hz, 2H), 4.62 (t, J = 8.0 Hz, 1H), 3.72-3.65 (m, 3H), 3.51-3.45 (m, 2H), 3.16 (bs, 2H), 2.91-2.73 (m, 2H), 2.22 (s, 3H), 1.36-1.60 (m, 2H); |
| 73 | 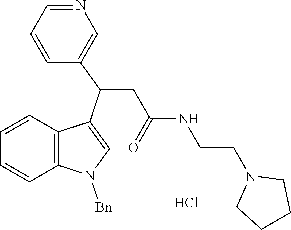 [SMILES: O=C(CC(c1cccnc1)c1cn(Cc2ccccc2)c2ccccc12)NCCN1CCCC1 ; Cl] | ¹H NMR (400 MHz, DMSO-d₆): δ 10.59 (bs, 1H), 8.92 (s, 1H), 8.68 (d, J = 5.2 Hz, 1H), 8.46-8.41 (m, 2H), 7.88- 7.85 (m, 1H), 7.63 (s, 1H), 7.39 (dd, J = 8.0 Hz, 2H), 7.31 (d, J = 6.8 Hz, 2H), 7.29-7.20 (m, 3H), 7.05 (t, J = 8.0 Hz, 1H), 6.93 (t, J = 7.2 Hz, 1H), 5.40 (s, 2H), 4.98 (t, J = 8.0 Hz, 1H), 3.44-3.35 (m, 2H), 3.34-3.31 (m, 2H), 3.11- 3.02 (m, 4H), 2.85-2.78 (m, 2H), 1.90-1.79 (m, 4H); |
| 74 | 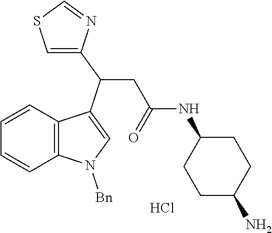 [SMILES: N[C@H]1CC[C@@H](NC(=O)CC(c2cscn2)c2cn(Cc3ccccc3)c3ccccc23)CC1 ; Cl] | ¹H NMR (400 MHz, DMSO-d₆): δ 8.99 (s, 1H), 7.93 (bs, 3H), 7.74 (d, J = 6.0 Hz, 1H), 7.49 (d, J = 8.0 Hz, 1H), 7.38- 7.23 (m, 7H), 7.14 (d, J = 6.8 Hz, 2H), 6.91 (t, J = 7.2 Hz, 1H), 5.36 (s, 2H), 4.91 (t, J = 7.6 Hz, 1H), 3.59 (bs, 1H), 3.07-2.91 (m, 3H), 1.51-1.60 (m, 6H), 1.42 (bs, 2H); |
| 75 | 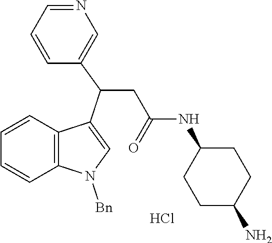 [SMILES: N[C@H]1CC[C@@H](NC(=O)CC(c2cccnc2)c2cn(Cc3ccccc3)c3ccccc23)CC1 ; Cl] | ¹H NMR (400 MHz, DMSO-d6): δ 8.93 (s, 1H), 8.71 (d, J = 4.0 Hz, 1H), 8.45 (d, J = 7.2 Hz, 1H), 8.03 (bs, 3H), 7.96- 7.95 (m, 2H), 7.60 (s, 1H), 7.39 (dd, J = 8.0 Hz, 2H), 7.32- 7.22 (m, 3H), 7.17 (d, J = 7.2 Hz, 2H), 7.07 (t, J = 7.6 Hz, 1H), 6.93 (t, J = 7.6 Hz, 1H), 5.36 (q, J = 16.0 Hz, 2H), 4.94 (t, J = 7.2 Hz, 1H), 3.58 (bs, 1H), 3.16-3.02 (m, 3H), 1.76- 1.58 (m, 4H), 1.53-1.40 (m, 4H); |
| 76 | 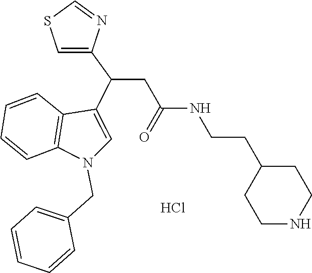 [SMILES: O=C(CC(c1cscn1)c1cn(Cc2ccccc2)c2ccccc12)NCCC1CCNCC1 ; Cl] | ¹H NMR (400 MHz, CD₃OD): 9.44 (d, J = 2.20 Hz, 1H), 7.52 (d, J = 7.80 Hz, 1H), 7.34 (d, J = 8.38 Hz, 1H), 7.37 (d, J = 8.24 Hz, 1H), 7.31-7.26 (m, 3H), 7.26-7.24 (m, 1H), 7.20-7.16 (m, 2H), 7.13 (t, J = 7.78 Hz, 1H), 7.03 (t J = 7.53 Hz, 1H), 5.36 (bs, 2H), 5.10 (t, J = 7.88 Hz, 1H), 3.20- 3.11 (m, 5H), 3.11-3.03 (m, 2H), 2.60-2.46 (m, 2H), 1.71 (d, J = 11.90 Hz, 1H), 1.59 (d, J = 11.23 Hz, 1H), 1.24-1.17 (m, 2, 1.15-1.08 (m, 2H);, |
| 77 | 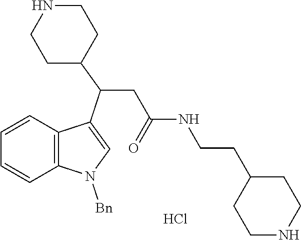 [SMILES: O=C(CC(c1cn(Cc2ccccc2)c2ccccc12)C1CCNCC1)NCCC1CCNCC1 ; Cl] | ¹H NMR (400 MHz, DMSO-d₆): 8.30 (bs, 2H), 7.76 (s, 1H), 7.55 (d, J = 7.2 Hz, 1H), 7.36-7.23 (m, 5H), 7.17-7.15 (m, 2H), 7.05-6.97 (m, 2H), 5.36 (s, 2H), 3.35 (bs, 2H), 3.19-3.08 (m, 5H), 2.93 (bs, 2H), 2.78-2.67 (m, 3H), 1.89-1.86 (m, 2H), 1.67-1.59 (m, 2H), 1.48-1.46 (m, 1H), 1.35-1.25 (m, 3H), 1.11 (bs, 5H); |
| 78 | 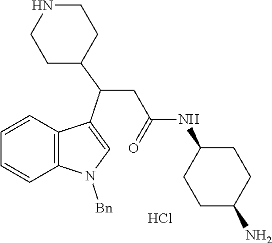 [SMILES: N[C@H]1CC[C@@H](NC(=O)CC(c2cn(Cc3ccccc3)c3ccccc23)C2CCNCC2)CC1 ; Cl] | ¹H NMR (400 MHz, DMSO-d₆): δ 8.71 (bs, 1H), 8.29 (bs, 1H), 7.97 (bs, 3H), 7.66 (d, J = 6.4 Hz, 1H), 7.57 (d, J = 8.0 Hz, 1H), 7.36-7.14 (m, 5H), 7.12 (d, J = 6.8 Hz, 2H), 7.07- 6.97 (m, 2H), 5.32 (q, J = 16.0 Hz, 2H), 3.70-3.61 (m, 1H), 3.41-3.31 (m, 1H), 3.19-3.22 (m, 2H), 3.01 (bs, 1H), 2.79-2.67 (m, 2H), 2.65-2.56 (m, 2H), 1.87-1.74 (m, 2H), 1.64-1.43 (m, 6H), 1.43-1.22 (m, 5H); |
| 79 | 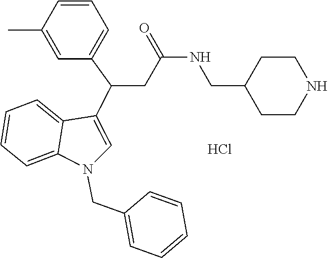 [SMILES: Cc1cccc(C(CC(=O)NCC2CCNCC2)c2cn(Cc3ccccc3)c3ccccc23)c1 ; Cl] | ¹H NMR (400 MHz, CD₃OD): δ 7.44 (d, J = 7.84 Hz, 1H), 7.31-7.21 (m, 5H), 7.19-7.12 (m, 5H), 7.06 (t, J = 7.68 Hz, 1H), 7.01-6.91 (m, 2H), 5.34 (bs, 2H), 4.73 (t, J = 8.34 Hz, 1H), 3.10-2.98 (m, 4H), 2.92-2.80 (m, 2H), 2.55-2.42 (m, 2H), 2.28 (bs, 3H), 1.32-1.30 (m, 3H), 1.08-0.947 (m, 2H); |
| 80 | 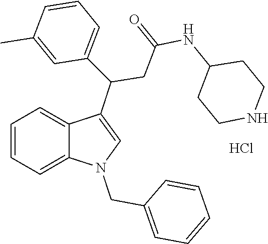 [SMILES: Cc1cccc(C(CC(=O)NC2CCNCC2)c2cn(Cc3ccccc3)c3ccccc23)c1 ; Cl] | ¹H NMR (400 MHz, CD₃OD): δ 7.35 (d, J = 7.84 Hz, 1H), 7.30-7.22 (m, 4H), 7.17 (bs, 1H), 7.14-7.08 (m, 5H), 7.05 (t, J = 7.58 Hz, 1H), 6.98 (d, J = 6.82 Hz, 1H), 6.91 (t, J = 7.33 Hz, 1H), 5.35 (bs, 2H), 4.72 (t, J = 8.35 Hz, 1H), 3.79-3.72 (m, 1H), 3.18-3.12 (m, 2H), 3.02-2.90 (m, 3H), 2.87-2.81 (m, 1H), 2.26 (bs, 3H), 1.82-1.71 (m, 2H), 1.45-1.36 (m, 2H); |
| 84 | 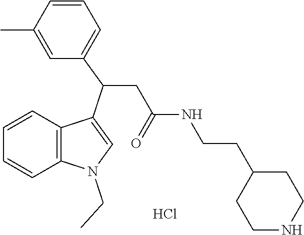 [SMILES: CCn1cc(C(CC(=O)NCCC2CCNCC2)c2cccc(C)c2)c2ccccc21 ; Cl] | ¹H NMR (400 MHz, DMSO-d₆): δ 8.47 (bs, 1H), 8.19 (bs, 1H), 7.82 (t, J = 5.6 Hz, 1H), 7.37 (t, J = 8.3 Hz, 2H), 7.29 (s, 1H), 7.15-7.05 (m, 4H), 6.96-6.88 (m, 2H), 4.62 (t, J = 7.9 Hz, 1H), 4.15 (q, J = 7.2 Hz, 2H), 3.19-3.11 (m, 3H), 3.04-2.94 (m, 2H), 2.89-2.81 (m, 1H), 2.78-2.70 (m, 1H), 2.59-2.52 (m, 2H), 2.23 (s, 3H), 1.70-1.56 (m, 2H), 1.34 (t, J = 7.2 Hz, 3H), 1.18-1.11 (m, 4H); |
| 88 | 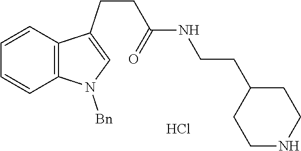 [SMILES: O=C(CCc1cn(Cc2ccccc2)c2ccccc12)NCCC1CCNCC1 ; Cl] | ¹H NMR (400 MHz, DMSO-d₆): δ 8.61 (bs, 1H), 8.35 (bs, 1H), 7.78 (t, J = 5.6 Hz, 1H), 7.53 (d, J = 7.6 Hz, 1H), 7.37 (d, J = 8.0 Hz, 1H), 7.31-7.15 (m, 4H), 7.07 (d, J = 8.0 Hz, 2H), 7.06 (t, J = 7.2 Hz, 1H), 6.97 (t, J = 7.2 Hz, 1H), 5.34 (s, 2H) 3.16 (d, J = 6.0 Hz, 2H), 3.03 (q, J = 6.8 Hz, 2H), 2.90 (t, J = 7.6 Hz, 2H), 2.68 (q, J = 12.0 Hz, 2H), 2.40 (t, J = 7.6 Hz, 2H), 1.72 (d, J = 12.0 Hz, 2H), 1.48-1.43 (m, 1H), 1.33-1.17 (m, 4H); |
| 89 | 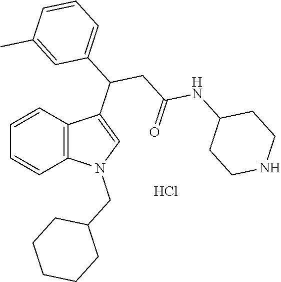 [SMILES: Cc1cccc(C(CC(=O)NC2CCNCC2)c2cn(CC3CCCCC3)c3ccccc23)c1 ; Cl] | ¹H NMR (400 MHz, DMSO-d₆): δ 8.44 (bs, 2H), 7.97 (d, J = 7.2 Hz, 1H), 7.37 (d, J = 7.9 Hz, 1H), 7.29 (d, J = 7.9 Hz, 1H) 7.25 (s, 1H), 7.13-6.99 (m, 4H), 6.95-6.90 (m, 1H), 6.87 (t, J = 7.2 Hz, 1H), 4.60 (t, J = 8.1 Hz, 1H), 4.01-3.90 (m, 2H), 3.78-3.64 (m, 1H), 3.20-3.04 (m, 2H), 2.95-2.80 (m, 3H), 2.77-2.68 (m, 1H), 2.21 (s, 3H), 1.84-1.30 (m, 10H), 1.20-0.89 (m, 5H); |
| 90 | 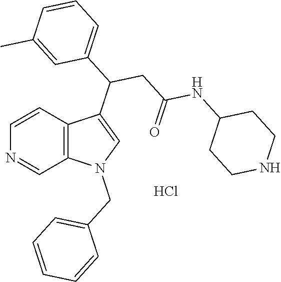 [SMILES: Cc1cccc(C(CC(=O)NC2CCNCC2)c2cn(Cc3ccccc3)c3cnccc23)c1 ; Cl] | ¹H NMR (400 MHz, Methanol-d₄): δ 9.16 (s, 1H), 8.24-8.20 (m, 1H), 8.16 (s, 1H), 7.79 (d, J = 6.7 Hz, 1H), 7.41 (s, 5H), 7.20-7.07 (m, 3H), 7.06-7.02 (m, 1H), 5.75 (s, 2H), 3.84- 3.75 (m, 1H), 3.25-3.17 (m, 2H), 3.11-2.93 (m, 4H), 2.27 (s, 3H), 1.93-1.85 (m, 1H), 1.84-1.76 (m, 1H), 1.62-1.50 (m, 1H), 1.48-1.36 (m, 1H), 1.33-1.16 (m, 1H); |
| 91 | 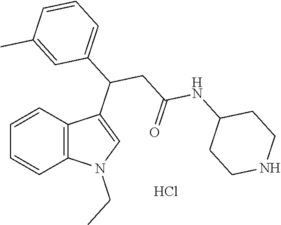 [SMILES: CCn1cc(C(CC(=O)NC2CCNCC2)c2cccc(C)c2)c2ccccc21 ; Cl] | ¹H NMR (400 MHz, DMSO-d₆): δ 8.60 (bs, 2H), 8.03 (d, J = 7.7 Hz, 1H), 7.38 (d, J = 8.2 Hz, 1H), 7.32 (t, J = 3.9 Hz, 2H) 7.14-7.02 (m, 4H), 6.96-6.85 (m, 2H), 4.60 (t, J = 7.9 Hz, 1H), 4.15 (q, J = 7.2 Hz, 2H), 3.78-3.66 (m, 1H), 3.20- 3.06 (m, 2H), 2.93-2.81 (m, 3H), 2.78-2.69 (m, 1H), 2.23 (s, 3H), 1.75-1.62 (m, 2H), 1.54-1.30 (m, 5H); |
| 92 | 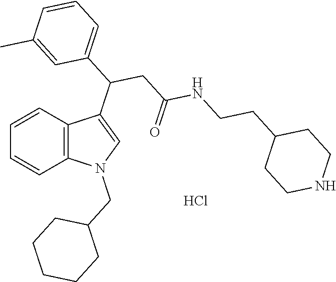 [SMILES: Cc1cccc(C(CC(=O)NCCC2CCNCC2)c2cn(CC3CCCCC3)c3ccccc23)c1 ; Cl] | ¹H NMR (400 MHz, DMSO-d₆): δ 8.17 (bs, 2H), 7.80 (m, 1H), 7.37 (d, J = 8.3 Hz, 1H), 7.32 (d, J = 7.3 Hz, 1H) 7.25 (s, 1H), 7.14-7.02 (m, 4H), 6.95-6.86 (m, 2H), 4.61 (t, J = 7.9 Hz, 1H), 3.95 (d, J = 7.2 Hz, 2H), 3.19-3.09 (m, 2H), 3.08-2.98 (m, 1H), 2.97-2.80 (m, 2H), 2.74-2.64 (m, 1H), 2.61-2.51 (m, 2H), 2.21 (s, 3H), 1.82-1.45 (m, 8H), 1.25- 0.90 (m, 10H); |
| 93 | 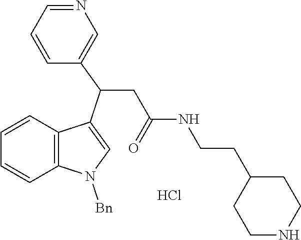 [SMILES: O=C(CC(c1cccnc1)c1cn(Cc2ccccc2)c2ccccc12)NCCC1CCNCC1 ; Cl] | ¹H NMR (400 MHz, DMSO-d6): δ 8.86 (bs, 1H), 8.77 (bs, 1H), 8.66 (bs, 1H), 8.53 (bs, 1H), 8.32 (s, 1H), 8.04 (s, 1H), 7.82 (s, 1H), 7.57 (s, 1H), 7.39 (d, J = 8.0 Hz, 2H), 7.32- 7.19 (m, 5H), 7.05 (t, J = 7.2 Hz, 1H), 6.93 (t, J = 7.2 Hz, 1H), 5.39 (s, 2H), 4.89 (t, J = 6.8 Hz, 1H), 3.14 (bs, 2H), 3.01-2.97 (m, 4H), 2.66-2.60 (m, 3H), 1.68-1.60 (m, 2H), 1.23-1.16 (m, 4H); |
| 94 | 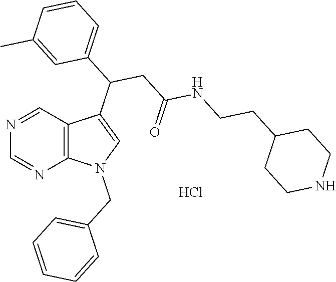 [SMILES: Cc1cccc(C(CC(=O)NCCC2CCNCC2)c2cn(Cc3ccccc3)c3ncncc23)c1 ; Cl] | ¹H NMR (400 MHz, DMSO-d₆): δ 9.13 (bs, 1H), 9.03 (bs, 1H), 8.76 (bs, 1H), 8.50 (bs, 1H), 7.93 (s, 2H), 7.39-7.23 (m, 5H), 7.22-7.11 (m, 3H), 7.05-6.95 (m, 1H), 5.60-5.43 (m, 2H), 4.76 (t, J = 7.8 Hz, 1H), 3.21-3.10 (m, 2H), 3.05-2.74 (m, 5H), 2.69-2.58 (m, 2H), 2.25 (s, 3H), 1.68-1.54 (m, 2H), 1.30-1.00 (m, 5H); |
| 96 | 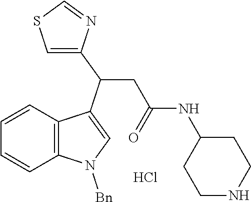 [SMILES: O=C(CC(c1cscn1)c1cn(Cc2ccccc2)c2ccccc12)NC1CCNCC1 ; Cl] | ¹H NMR (400 MHz, DMSO-d₆): δ 8.90 (s, 1H), 8.70 (bs, 2H), 8.05 (d, J = 7.2 Hz, 1H), 7.48 (d, J = 8.0 Hz, 1H), 7.39- 7.36 (m, 3H), 7.30-7.15 (m, 3H), 7.05 (d, J = 7.6 Hz, 1H), 7.03 (t, J = 7.2 Hz, 1H), 6.92 (t, J = 7.2 Hz, 1H), 5.35 (s, 2H), 4.91 (t, J = 7.6 Hz, 1H), 3.50-3.46 (m, 1H), 3.12 (bs, 2H), 2.99-2.37 (m, 4H), 1.72-1.59 (m, 2H), 1.49- 1.41 (m, 2H); |
| 97 | 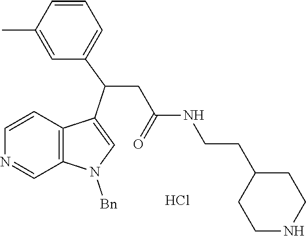 [SMILES: Cc1cccc(C(CC(=O)NCCC2CCNCC2)c2cn(Cc3ccccc3)c3cnccc23)c1 ; Cl] | ¹H NMR (400 MHz, Methanol-d₄): δ 9.15 (s, 1H), 8.24-8.20 (m, 1H), 8.12 (s, 1H), 7.82 (d, J = 6.8 Hz, 1H), 7.44-7.38 (m, 5H), 7.22-7.11 (m, 3H), 7.07-7.03 (m, 1H), 5.75 (s, 2H), 3.09-2.94 (m, 4H), 2.86-2.75 (m, 2H), 2.29 (s, 3H), 1.87- 1.77 (m, 2H), 1.40-1.34 (m, 4H), 1.32-1.25 (m, 4H); |
| 98 | 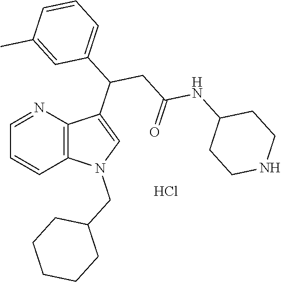 [SMILES: Cc1cccc(C(CC(=O)NC2CCNCC2)c2cn(CC3CCCCC3)c3cccnc23)c1 ; Cl] | ¹H NMR (400 MHz, DMSO-d₆): δ 8.88-8.26 (m, 5H), 8.00 (bs, 1H), 7.59 (bs, 1H), 7.37-7.12 (m, 8H) 7.02-6.96 (m, 1H), 5.62 (bs, 2H), 5.00 (t, J = 7.8 Hz, 1H), 3.20-3.10 (m, 2H), 3.07-2.92 (m, 2H), 2.91-2.76 (m, 2H), 2.70-2.55 (m, 2H), 2.25 (s, 3H), 1.61 (t, J = 10.3 Hz, 2H), 1.31-1.24 (m, 1H), 1.22-1.05 (m, 4H); |
| 99 | 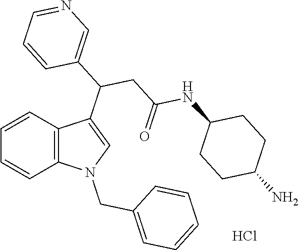 [SMILES: N[C@H]1CC[C@H](NC(=O)CC(c2cccnc2)c2cn(Cc3ccccc3)c3ccccc23)CC1 ; Cl] | ¹H NMR 400 MHz; DMSO-d₆): δ 8.77 (brs, 1H), 8.64 (d, J = 5.0 Hz, 1H), 8.50 (d, J = 8.0 Hz, 1H), 8.04-7.88 (m, 4H), 7.81 (t, J = 7.2 Hz, 1H), 7.39 (s, 1H), 7.38-7.32 (m, 2H), 7.31-7.22 (m, 3H), 7.18-7.08 (m, 3H), 7.01-6.95 (m, 1H), 5.45-5.34 (m, 2H), 5.04 (t, J = 7.7 Hz, 1H), 3.05-2.93 (m, 4H), 1.84-1.73 (m, 2H), 1.72-1.63 (m, 2H), 1.46-1.27 (m, 2H), 1.24-1.04 (m, 2H); |
| 100 | 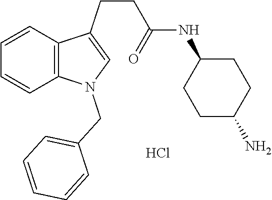 [SMILES: N[C@H]1CC[C@H](NC(=O)CCc2cn(Cc3ccccc3)c3ccccc23)CC1 ; Cl] | ¹H NMR (400 MHz, DMSO-d₆): δ 7.87-7.84 (m, 3H), 7.73 (d, J = 7.6 Hz, 1H), 7.54 (d, J = 7.8 Hz, 1H), 7.39 (d, J = 8.2 Hz, 1H), 7.32-7.22 (m, 3H), 7.21-7.15 (m, 3H), 7.12-7.05 (m, 1H), 7.02-6.96 (m, 1H), 5.33 (s, 2H), 3.47-3.42 (m, 1H), 2.91 (t, J = 7.5 Hz, 3H), 2.39 (t, J = 7.5 Hz, 2H), 1.94-1.86 (m, 2H), 1.80-1.72 (m, 2H), 1.42-1.27 (m, 2H), 1.19-1.06 (m, 2H); |
| 101 | 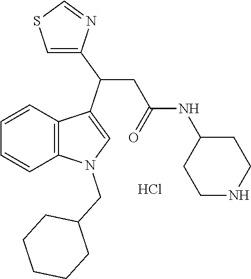 [SMILES: O=C(CC(c1cscn1)c1cn(CC2CCCCC2)c2ccccc12)NC1CCNCC1 ; Cl] | ¹H NMR (400 MHz, Methanol-d₄): δ 8.95-8.91 (m, 1H), 7.47 (d, J = 8.0 Hz, 1H), 7.32 (d, J = 8.3 Hz, 1H), 7.22-7.20 (m, 1H), 7.14-7.06 (m, 2H), 6.99-6.93 (m, 1H), 5.00 (t, J = 8.0 Hz, 1H), 3.94 (d, J = 7.2 Hz, 2H), 3.82-3.71 (m, 1H), 3.16-3.07 (m, 2H), 3.04-2.87 (m, 3H), 1.96-1.80 (m, 2H), 1.76-1.65 (m, 4H), 1.63-1.55 (m, 2H), 1.53-1.28 (m, 3H), 1.25-1.15 (m, 3H), 1.11-0.96 (m, 2H); |
| 102 | 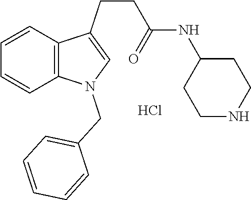 [SMILES: O=C(CCc1cn(Cc2ccccc2)c2ccccc12)NC1CCNCC1 ; Cl] | ¹H NMR (400 MHz, DMSO-d₆): δ 8.59-8.74 (m, 2H), 7.97 (d, J = 7.4, 1H), 7.54 (d, J = 7.8, 1H), 7.39 (d, J = 8.5 Hz, 1H), 7.33-7.27 (m, 2H), 7.26-7.21 (m, 2H), 7.19-7.15 (m, 2H), 7.11-7.06 (m, 1H), 7.02-6.97 (m, 1H), 5.34 (s, 2H), 3.87-3.76 (m, 1H), 3.23-3.16 (m, 2H), 2.96-2.90 (m, 4H), 2.43 (t, J = 7.6 Hz, 2H), 1.85-1.77 (m, 2H), 1.59-1.46 (m, 2H); |
| 103 | 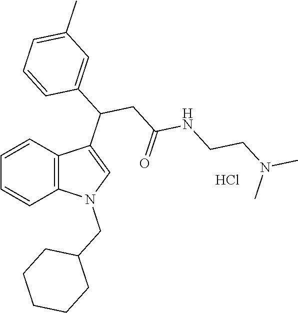 [SMILES: Cc1cccc(C(CC(=O)NCCN(C)C)c2cn(CC3CCCCC3)c3ccccc23)c1 ; Cl] | ¹H NMR (400 MHz, Methanol-d₄): δ 7.36 (d, J = 8.0 Hz, 1H), 7.31 (d, J = 8.3 Hz, 1H), 7.18-7.05 (m, 5H), 6.99 (d, J = 6.9 Hz, 1H), 6.95-6.89 (m, 1H), 4.72 (t, J = 8.1 Hz, 1H), 3.96 (d, J = 7.2 Hz, 2H), 3.43-3.35 (m, 2H), 3.09-3.02 (m, 1H), 3.01-2.96 (m, 2H), 2.95-2.87 (m, 1H), 2.76 (s, 3H), 2.74 (s, 3H), 2.27 (s, 3H), 1.89-1.78 (m, 1H), 1.77-1.64 (m, 3H), 1.63-1.54 (m, 2H), 1.26-1.17 (m, 3H), 1.08-0.96 (m, 2H); |
| 104 | 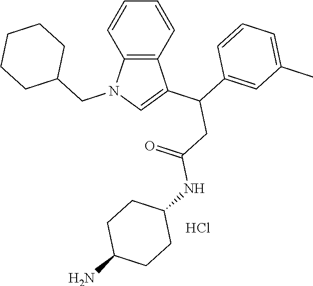 [SMILES: Cc1cccc(C(CC(=O)N[C@H]2CC[C@H](N)CC2)c2cn(CC3CCCCC3)c3ccccc23)c1 ; Cl] | ¹H NMR (400 MHz, Methanol-d₄): δ 7.30 (t, J = 8.8 Hz, 2H), 7.14-7.04 (m, 5H), 6.96 (t, J = 7.5 Hz, 1H), 6.91-6.86 (m, 1H), 4.69 (t, J = 8.1 Hz, 1H), 3.96 (d, J = 7.2 Hz, 2H), 3.04-2.92 (m, 2H), 2.84-2.75 (m, 1H), 2.25 (s, 3H), 2.00- 1.91 (m, 2H), 1.90-1.81 (m, 1H), 1.80-1.65 (m, 5H), 1.64- 1.54 (m, 2H), 1.45-1.32 (m, 2H), 1.27-1.17 (m, 4H), 1.16- 0.95 (m, 4H; |
| 106 | 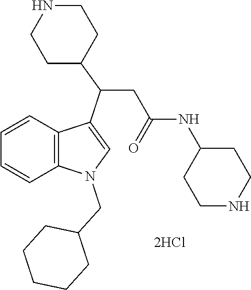 [SMILES: O=C(CC(c1cn(CC2CCCCC2)c2ccccc12)C1CCNCC1)NC1CCNCC1] | ¹H NMR (400 MHz, DMSO-d₆): δ 8.75 (bs, 3H), 8.27 (bs, 1H), 7.98 (d, J = 7.6 Hz, 1H), 7.53 (d, J = 8.0 Hz, 1H), 7.37 (d, J = 8.0 Hz, 1H), 7.11-7.06 (m, 2H), 6.95 (t, J = 7.2 Hz, 1H), 3.93 (d, J = 7.2 Hz, 1H), 3.66-3.69 (m, 2H), 3.35- 3.30 (m, 1H), 3.33-3.07 (m, 4H), 2.91-2.67 (m, 5H), 1.86- 1.75 (m, 4H), 1.49-1.64 (m, 9H), 1.39-1.33 (m, 3H), 1.21-0.95 (m, 5H); |
| 107 | 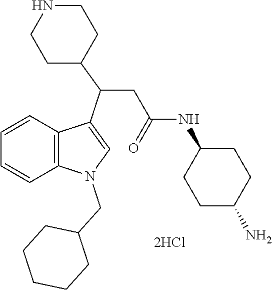 [SMILES: N[C@H]1CC[C@H](NC(=O)CC(c2cn(CC3CCCCC3)c3ccccc23)C2CCNCC2)CC1] | ¹H NMR (400 MHz, DMSO-d₆): δ 8.75 (bs, 1H), 8.27 (bs, 1H), 7.94 (bs, 3H), 7.67 (d, J = 7.2 Hz, 1H), 7.52 (d, J = 8.0 Hz, 1H), 7.37 (d, J = 8.0 Hz, 1H), 7.24-7.06 (m, 2H), 6.95 (t, J = 7.2 Hz, 1H), 3.92 (s, 2H), 3.34-3.18 (m, 3H), 2.87- 2.69 (m, 4H), 1.90-1.83 (m, 8H), 1.74-1.45 (m, 6H), 1.33- 1.24 (m, 4H), 1.12-0.95 (m, 8H); |
| 108 | 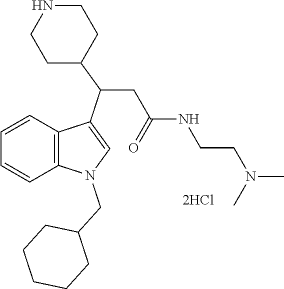 [SMILES: CN(C)CCNC(=O)CC(c1cn(CC2CCCCC2)c2ccccc12)C1CCNCC1] | ¹H NMR (400 MHz, DMSO-d₆): δ 10.24 (bs, 1H), 8.72 (bs, 1H), 8.25 (bs, 2H), 7.53 (d, J = 7.6 Hz, 1H), 7.38 (d, J = 8.0 Hz, 1H), 7.15 (s, 1H), 7.07 (t, J = 7.2 Hz, 1H), 6.98 (t, J = 7.2 Hz, 1H), 3.95 (s, 2H), 3.32-3.17 (m, 4H), 2.95-2.93 (m, 2H), 2.79-2.72 (m, 2H), 2.63 (s, 6H), 2.62-2.58 (m, 2H), 1.87-1.84 (m, 2H), 1.76 (bs, 1H), 1.64-1.49 (m, 6H), 1.29 (t, J = 8.0 Hz, 2H), 1.13-0.95 (m, 6H); |
| 109 | 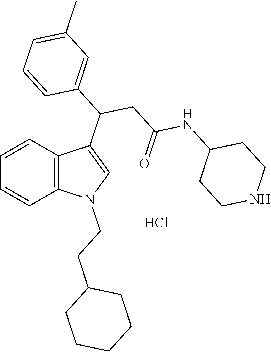 [SMILES: Cc1cccc(C(CC(=O)NC2CCNCC2)c2cn(CCC3CCCCC3)c3ccccc23)c1 ; Cl] | ¹H NMR (400 MHz, DMSO): δ 8.45 (bs, 1H), 8.36 (bs, 1H), 7.96 (d, J = 9.0 Hz, 1H), 7.37-7.27 (m, 3H), 7.12-7.03 (m, 4H), 6.94-6.89 (m, 2H), 4.60 (t, J = 8.2 Hz, 1H), 4.20- 4.07(m, 2H), 3.74-3.69 (m, 1H), 3.17-3.07 (m, 2H), 2.94- 2.67 (m, 4H), 2.22 (s, 3H), 1.79-1.59 (m, 9H), 1.50-1.40 (m, 2H), 1.23-1.09 (m, 4H), 1.00-0.87 (m, 2H). |
| 110 | 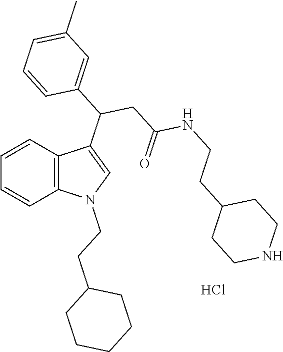 [SMILES: Cc1cccc(C(CC(=O)NCCC2CCNCC2)c2cn(CCC3CCCCC3)c3ccccc23)c1 ; Cl] | ¹H NMR (400 MHz, DMSO-d₆): δ 8.47 (bs, 1H), 8.17 (bs, 1H), 7.81 (t, J = 6.3 Hz, 1H), 7.35 (d, J = 8.4 Hz, 2H), 7.28 (s, 1H), 7.13-7.04 (m, 4H), 6.94-6.88 (m, 2H), 4.61 (t, J = 7.7 Hz, 1H), 4.16-4.09 (m, 2H), 3.20-3.14 (m, 2H), 3.05- 2.92 (m, 2H), 2.86-2.81 (m, 1H), 2.75-2.65 (m, 1H), 2.22 (s, 3H), 1.78-1.60 (m, 9H), 1.22-1.06 (m, 10H), 0.99-0.88 (m, 3H). |
| 111 | 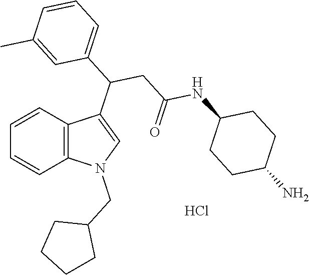 [SMILES: Cc1cccc(C(CC(=O)N[C@H]2CC[C@H](N)CC2)c2cn(CC3CCCC3)c3ccccc23)c1 ; Cl] | ¹H NMR 400 MHz; DMSO-d₆): δ 7.82-7.64 (m, 4H), 7.39 (d, J = 8.6 Hz, 1H), 7.32-7.25 (m, 2H), 7.13-7.00 (m, 4H), 6.94-6.84 (m, 2H), 4.60 (t, J = 8.0 Hz, 1H), 4.03 (d, J = 7.4 Hz, 2H), 3.40-3.34 (m, 1H), 2.98-2.79 (m, 2H), 2.71-2.62 (m, 1H), 2.39-2.29 (m, 1H), 2.21 (s, 3H), 1.90-1.79 (m, 2H), 1.69-1.43 (m, 8H), 1.34-1.20 (m, 4H), 1.18-1.00 (m, 2H). |
| 112 | 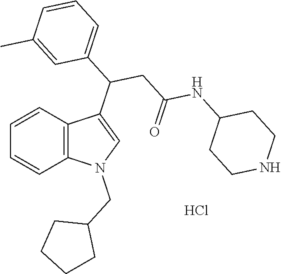 [SMILES: Cc1cccc(C(CC(=O)NC2CCNCC2)c2cn(CC3CCCC3)c3ccccc23)c1 ; Cl] | ¹H NMR (400 MHz, DMSO-d₆): δ 8.41 (bs, 2H), 7.97 (d, J = 7.5 Hz, 1H), 7.40 (d, J = 8.3 Hz, 1H), 7.30 (d, J = 8.2 Hz, 2H), 7.14-7.01 (m, 4H), 6.97-6.84 (m, 2H), 4.61 (t, J = 7.9 Hz, 1H), 4.11-3.97 (m, 2H), 3.78-3.68 (m, 1H), 3.21-3.08 (m, 2H), 2.94-2.82 (m, 3H), 2.79-2.69 (m, 1H), 2.38-2.31 (m, 1H), 2.22 (s, 3H), 1.72-1.55 (m, 6H), 1.53-1.45 (m, 3H), 1.43-1.34 (m, 1H), 1.30-1.22 (m, 2H); |
| 113 | 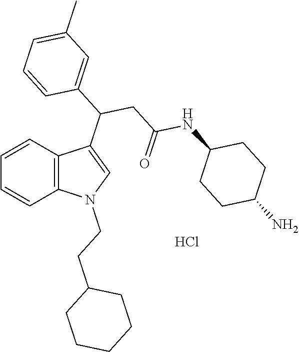 [SMILES: Cc1cccc(C(CC(=O)N[C@H]2CC[C@H](N)CC2)c2cn(CCC3CCCCC3)c3ccccc23)c1 ; Cl] | ¹H NMR (400 MHz, DMSO-d₆): δ 7.76 (d, J = 8.0 Hz, 1H), 7.71 (bs, 3H), 7.35 (d, J = 8.3 Hz, 1H), 7.30 (d, J = 10.3 Hz, 2H), 7.11-7.03 (m, 4H), 6.93-6.86 (m, 2H), 4.60 (t, J = 8.2 Hz, 1H), 4.13 (t, J = 7.1 Hz, 2H), 3.38-3.33 (m, 1H), 2.94- 2.78 (m, 2H), 2.70-2.64 (m, 1H), 2.21 (s, 3H), 1.89-1.78 (m, 3H), 1.70-1.57 (m, 8H), 1.33-1.21 (m, 2H), 1.21-1.02 (m, 6H), 0.98-0.88 (m, 2H). |
| 114 | 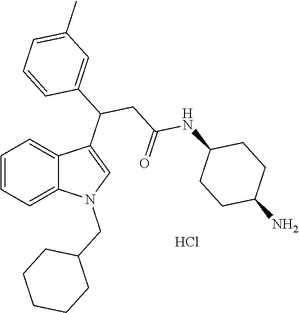 [SMILES: Cc1cccc(C(CC(=O)N[C@H]2CC[C@@H](N)CC2)c2cn(CC3CCCCC3)c3ccccc23)c1 ; Cl] | ¹H NMR 400 MHz; DMSO-d₆): δ 7.84 (bs, 3H), 7.72 (d, J = 6.2 Hz, 1H), 7.37 (d, J = 8.3 Hz, 1H), 7.28 (d, J = 7.8 Hz, 1H), 7.26 (s, 1H), 7.10 (t, J = 7.5 Hz, 1H), 7.08-7.01 (m, 3H), 6.92 (d, J = 7.5 Hz, 1H), 6.87 (t, J = 7.2 Hz, 1H), 4.61 (t, J = 7.9 Hz, 1H), 4.02-3.89 (m, 2H), 3.63-3.54 (m, 1H), 3.10-2.98 (m, 1H), 2.96-2.87 (m, 1H), 2.79-2.70 (m, 1H), 2.21 (s, 3H), 1.81-1.36 (m, 14H), 1.20-1.04 (m, 3H), 1.03- 0.90 (m, 2H). |
| 115 | 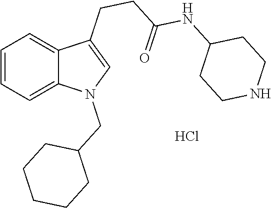 [SMILES: O=C(CCc1cn(CC2CCCCC2)c2ccccc12)NC1CCNCC1 ; Cl] | ¹H NMR 400 MHz; DMSO-d₆): δ 8.81 (bs, 2H), 8.00 (d, J = 7.4 Hz, 1H), 7.51 (d, J = 7.7 Hz, 1H), 7.38 (d, J = 8.0 Hz, 1H), 7.09 (t, J = 7.7 Hz, 1H), 7.05 (s, 1H), 6.97 (t, J = 7.4 Hz, 1H), 3.92 (d, J = 7.1 Hz, 2H), 3.86-3.78 (m, 1H), 3.25- 3.13 (m, 2H), 3.01-2.83 (m, 4H), 2.42 (t, J = 7.5 Hz, 2H), 1.90-1.44 (m, 10H), 1.19-0.89 (m, 5H). |
| 116 | 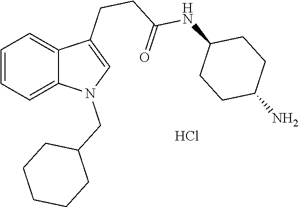 [SMILES: N[C@H]1CC[C@H](NC(=O)CCc2cn(CC3CCCCC3)c3ccccc23)CC1 ; Cl] | ¹H NMR 400 MHz; DMSO-d₆): δ 7.92 (bs, 3H), 7.73 (d, J = 7.4 Hz, 1H), 7.50 (d, J = 7.7 Hz, 1H), 7.38 (d, J = 8.2 Hz, 1H), 7.08 (t, J = 7.4 Hz, 1H), 7.04 (s, 1H), 6.97 (t, J = 7.4 Hz, 1H), 3.91 (d, J = 7.1 Hz, 2H), 3.51-3.40 (m, 1H), 3.01- 2.82 (m, 3H), 2.38 (t, J = 7.4 Hz, 2H), 1.97-1.86 (m, 2H), 1.82-1.56 (m, 6H), 1.54-1.45 (m, 2H), 1.41-1.28 (m, 2H), 1.22-1.06 (m, 5H), 1.02-0.89 (m, 2H). |
| 117 | 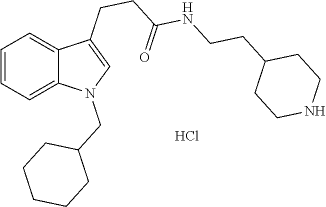 [SMILES: O=C(CCc1cn(CC2CCCCC2)c2ccccc12)NCCC1CCNCC1 ; Cl] | ¹H NMR 400 MHz; DMSO-d₆): δ 8.74 (bs, 1H), 8.49 (bs, 1H), 7.80 (d, J = 5.2 Hz, 1H), 7.51 (d, J = 8.2 Hz, 1H), 7.38 (d, J = 8.2 Hz, 1H), 7.09 (t, J = 7.4 Hz, 1H), 7.06 (s, 1H), 6.97 (t, J = 7.4 Hz, 1H), 3.92 (d, J = 7.1 Hz, 2H), 3.19 (d, J = 12.2 Hz, 2H), 3.05 (q, J = 6.7 Hz, 2H), 2.89 (t, J = 7.4 Hz, 2H), 2.72 (q, J = 12.2 Hz, 2H), 2.40 (t, J = 8.2 Hz, 2H), 1.80-1.39 (m, 9H), 1.35-1.18 (m, 4H), 1.16-1.05 (m, 3H), 1.04-0.88 (m, 2H). |
| 118 | 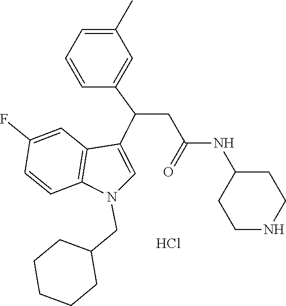 [SMILES: Cc1cccc(C(CC(=O)NC2CCNCC2)c2cn(CC3CCCCC3)c3ccc(F)cc23)c1 ; Cl] | ¹H NMR (400 MHz, DMSO-d₆): δ 8.41 (bs, 1H), 8.35 (bs, 1H), 7.96 (d, J = 7.6 Hz, 1H), 7.42-7.38 (m, 1H), 7.34 (s, 1H), 7.13-7.04 (m, 3H), 7.0-6.86 (m, 3H), 4.53 (t, J = 8.0 Hz, 1H), 3.99-3.91 (m, 2H), 3.75-3.68 (m, 1H), 3.17- 2.91 (m, 2H), 2.89-2.78 (m, 3H), 2.73-2.70 (m, 1H), 2.22 (s, 3H), 1.76-1.61 (m, 6H), 1.51-1.31 (m, 4H), 1.38- 0.94 (m, 5H); |
| 119 | 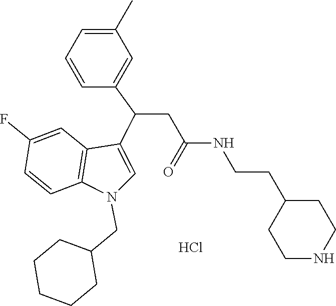 [SMILES: Cc1cccc(C(CC(=O)NCCC2CCNCC2)c2cn(CC3CCCCC3)c3ccc(F)cc23)c1 ; Cl] | ¹H NMR (400 MHz, DMSO-d₆): δ 8.57 (bs, 1H), 8.28 (bs, 1H), 7.81 (t, J = 6.0 Hz, 1H), 7.41-7.38 (m, 1H), 7.14 (s, 1H), 7.13-7.11 (m, 3H), 7.07 (d, J = 6.4 Hz, 1H), 6.95- 6.86 (m, 2H), 4.54 (t, J = 8.0 Hz, 1H), 3.94 (d, J = 7.2 Hz, 2H), 3.17-3.14 (m, 2H), 2.80-3.05 (m, 3H), 2.74-2.72 (m, 1H), 2.68-2.59 (m, 2H), 2.22 (s, 3H), 1.77-1.60 (m, 6H), 1.51-1.47 (m, 2H), 1.24-1.11 (m, 8H), 1.0-0.94 (m, 2H); |
| 120 | 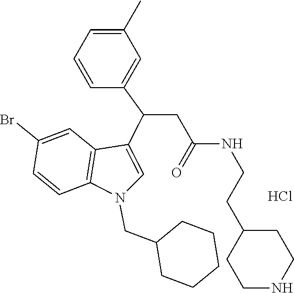 [SMILES: Cc1cccc(C(CC(=O)NCCC2CCNCC2)c2cn(CC3CCCCC3)c3ccc(Br)cc23)c1 ; Cl] | ¹H NMR (400 MHz, DMSO-d₆): δ 8.49 (bs, 1H), 8.20 (bs, 1H), 7.83-7.80 (m, 1H), 7.46-7.45 (m, 1H), 7.39 (d, J = 8.4 Hz, 1H), 7.32 (s, 1H), 7.17-7.12 (m, 2H), 7.07 (d, J = 7.8 Hz, 2H), 6.95 (d, J = 7.6 Hz, 1H), 4.59 (t, J = 8.1 Hz, 1H), 3.96 (d, J = 7.55 Hz, 1H), 3.18-3.15 (m, 2H), 3.05-2.99 (m, 2H), 2.95-2.87 (m, 1H), 2.84-2.79 (m, 1H), 2.74-2.51 (m, 3H), 2.22 (s, 3H), 1.78-1.43 (m, 8H), 1.23-0.90 (m, 10H). |
| 121 | 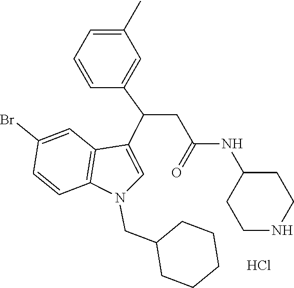 [SMILES: Cc1cccc(C(CC(=O)NC2CCNCC2)c2cn(CC3CCCCC3)c3ccc(Br)cc23)c1 ; Cl] | ¹H NMR (400 MHz, DMSO-d₆): δ 8.45 (bs, 2H), 7.98-7.96 (m, 1H), 7.42-7.39 (m, 2H), 7.33 (s, 1H), 7.17-7.10 (m, 2H), 7.06-7.04 (m, 2H), 6.95 (d, J = 7.1, 1H), 4.59 (t, J = 7.9 Hz, 1H), 4.00-3.93 (m, 2H), 3.75-3.67 (m, 1H), 3.18- 3.07 (m, 2H), 2.93-2.59 (m, 4H), 2.23 (s, 3H), 1.74-1.57 (m, 6H), 1.51-1.36 (m, 4H), 1.16-1.06 (m, 3H), 1.00-0.92 (m, 2H). |
| 123 | 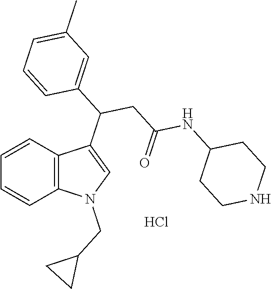 [SMILES: Cc1cccc(C(CC(=O)NC2CCNCC2)c2cn(CC3CC3)c3ccccc23)c1 ; Cl] | ¹H NMR (400 MHz, Methanol-d₄): δ 7.36-7.32 (m, 2H), 7.16 (s, 1H), 7.14-7.07 (m, 4H), 7.00-6.96 (m, 1H), 6.93- 6.88 (m, 1H), 4.69 (t, J = 8.0 Hz, 1H), 4.01 (d, J = 6.7 Hz, 2H), 3.84-3.75 (m, 1H), 3.22-3.12 (m, 2H), 3.04-2.93 (m, 3H), 2.91-2.83 (m, 1H), 2.27 (s, 3H), 1.89-1.76 (m, 2H), 1.55-1.34 (m, 2H), 1.31-1.22 (m, 1H), 0.61-0.54 (m, 2H), 0.39-0.34 (m, 2H); |
| 124 | 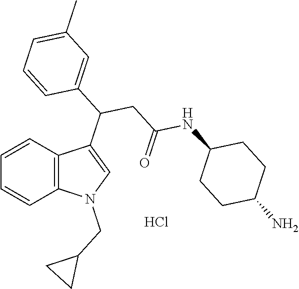 [SMILES: Cc1cccc(C(CC(=O)N[C@H]2CC[C@H](N)CC2)c2cn(CC3CC3)c3ccccc23)c1 ; Cl] | ¹H NMR (400 MHz, Methanol-d₄): δ 7.36-7.31 (m, 2H), 7.16-7.06 (m, 5H), 6.99-6.95 (m, 1H), 6.90 (t, J = 7.7 Hz, 1H), 4.68 (t, J = 8.0 Hz, 1H), 4.01 (d, J = 6.7 Hz, 2H), 3.02- 2.93 (m, 2H), 2.85-2.78 (m, 1H), 2.26 (s, 3H), 1.98-1.91 (m, 2H), 1.80-1.66 (m, 2H), 1.45-1.32 (m, 2H), 1.31-1.19 (m, 2H), 1.18-1.00 (m, 2H) 0.58-0.54 (m,. 2H), 0.38-0.34 (m,. 2H); |
| 125 | 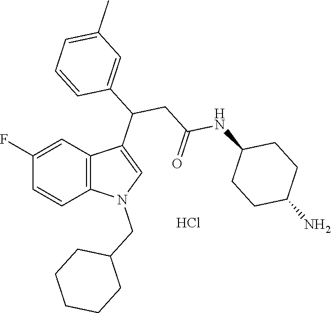 [SMILES: Cc1cccc(C(CC(=O)N[C@H]2CC[C@H](N)CC2)c2cn(CC3CCCCC3)c3ccc(F)cc23)c1 ; Cl] | ¹H NMR (400 MHz, DMSO-d₆): δ 7.95 (d, J = 7.6 Hz, 1H), 7.80 (bs, 3H), 7.38 (d, J = 4.4 Hz, 1H), 7.33 (s, 1H), 7.09 (t, J = 7.2 Hz, 1H), 7.06 (bs, 1H), 7.03 (bs, 1H), 6.97 (dd, J = 10.0, 2.4 Hz, 1H), 6.98-6.86 (m, 2H), 4.53 (t, J = 8.0 Hz, 1H), 3.94 (d, J = 7.2 Hz, 2H), 2.92-2.80 (m, 2H), 2.68- 2.65 (m, 1H), 2.21 (s, 3H), 1.90-1.84 (m, 2H), 1.75-1.52 (m, 6H), 1.46 (t, J = 12.4 Hz, 2H), 1.33-1.25 (m, 3H), 1.28- 0.94 (m, 7H); |
| 126 | 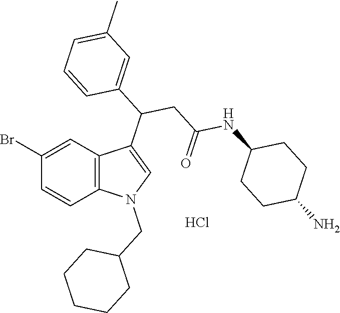 [SMILES: Cc1cccc(C(CC(=O)N[C@H]2CC[C@H](N)CC2)c2cn(CC3CCCCC3)c3ccc(Br)cc23)c1 ; Cl] | ¹H NMR (400 MHz, DMSO-d₆): δ 7.76 (d, J = 8.0 Hz, 1H), 7.71 (bs, 3H), 7.42-7.38 (m, 2H), 7.32 (s, 1H), 7.17-7.10 (m, 2H), 7.05-7.03 (m, 2H), 6.94 (d, J = 7.4 Hz, 1H), 4.58 (t, J = 8.7 Hz, 1H), 3.96 (d, J = 6.7 Hz, 2H), 3.37-3.32 (m, 1H), 2.95-2.88 (m, 1H), 2.84-2.78 (m, 1H), 2.68-2.62 (m, 1H), 2.22 (s, 3H), 1.88-1.82 (m, 2H), 1.77-1.58 (m, 6H), 1.51- 1.41 (m, 2H), 1.34-1.23 (m, 2H), 1.16-0.93 (m, 7H). |
| 127 | 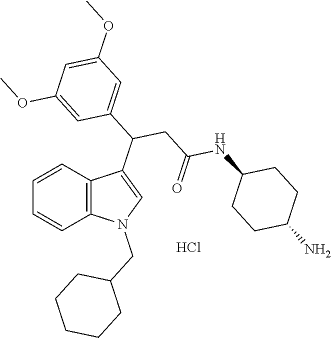 [SMILES: COc1cc(OC)cc(C(CC(=O)N[C@H]2CC[C@H](N)CC2)c2cn(CC3CCCCC3)c3ccccc23)c1 ; Cl] | ¹H NMR (400 MHz, DMSO-d₆): δ 7.82-7.69 (m, 4H), 7.40- 7.32 (m, 2H), 7.26 (s, 1H), 7.05 (t, J = 7.5 Hz, 1H), 6.89 (t, J = 7.4 Hz, 1H), 6.39 (m, 2H), 6.27-6.24 (m, 1H), 4.58 (t, J = 7.7 Hz, 1H), 3.99-3.93 (m, 2H), 3.65 (s, 6H), 2.98-2.88 (m, 1H), 2.87-2.78 (m, 1H), 2.72-2.63 (m, 1H), 1.92-1.83 (m, 2H), 1.81-1.60 (m, 6H), 1.57-1.45 (m, 3H), 1.36-1.23 (m, 2H), 1.21-1.06 (m, 5H), 1.05-0.93 (m, 2H); |
| 128 | 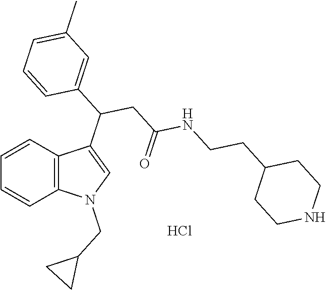 [SMILES: Cc1cccc(C(CC(=O)NCCC2CCNCC2)c2cn(CC3CC3)c3ccccc23)c1 ; Cl] | ¹H NMR (400 MHz, Methanol-d₄): δ 7.43 (d, J = 7.7 Hz, 1H), 7.36 (d, J = 8.3 Hz, 1H), 7.22 (s, 1H), 7.17-7.09 (m, 4H), 7.00-6.92 (m, 2H), 4.75-4.70 (m, 1H), 4.01 (t, J = 6.7 Hz, 2H), 3.22-3.11 (m, 3H), 3.03-2.96 (m, 1H), 2.89- 2.82 (m, 1H), 2.53-2.43 (m, 1H), 2.42-2.33 (m, 1H), 2,28 (s, 3H), 1.81-1.74 (m, 1H), 1.67-1.59 (m, 1H), 1.40-1.34 (m, 2H), 1.28-1.22 (m, 2H), 1.20-1.08 (m, 2H), 1.06-0.96 (m, 1H), 0.61-0.55 (m, 2H), 0.42-0.35 (m, 2H); |
| 130 | 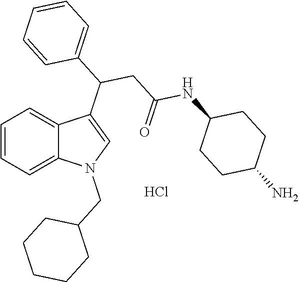 [SMILES: N[C@H]1CC[C@H](NC(=O)CC(c2ccccc2)c2cn(CC3CCCCC3)c3ccccc23)CC1 ; Cl] | ¹H NMR (400 MHz; DMSO-d₆): δ 7.88 (bs, 3H), 7.80 (d, J = 7.8 Hz, 1H), 7.36 (d, J = 8.2 Hz, 1H), 7.31-7.17 (m, 6H), 7.14-7.08 (m, 1H), 7.07-7.00 (m, 1H), 6.89-6.82 (m, 1H), 4.64 (t, J = 7.8 Hz, 1H), 4.00-3.88 (m, 2H), 3.42-3.34 (m, 1H), 2.96-2.81 (m, 2H), 2.73-2.62 (m, 1H), 1.93-1.46 (m, 10H), 1.36-1.22 (m, 2H), 1.20-0.91 (m, 7H). |
| 131 | 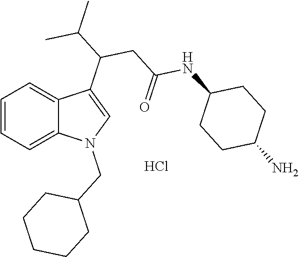 [SMILES: CC(C)C(CC(=O)N[C@H]1CC[C@H](N)CC1)c1cn(CC2CCCCC2)c2ccccc12 ; Cl] | ¹H NMR (400 MHz, Methanol-d₄): δ 7.56 (d, J = 8.0 Hz, 1H), 7.29 (d, J = 8.2 Hz, 1H), 7.11-7.06 (m, 1H), 7.01-6.93 (m, 2H), 3.96-3.90 (m, 2H), 3.23-3.19 (m, 1H), 2.95-2.86 (m, 1H), 2.71-2.63 (m, 1H), 2.56-2.47 (m, 1H), 2.06-1.98 (m, 1H), 1.96-1.89 (m, 1H), 1.86-1.78 (m, 2H), 1.77-1.65 (m, 4H), 1.63-1.55 (m, 2H), 1.40-1.28 (m, 4H), 1.27-1.16 (m, 4H), 1.14-1.08 (m, 1H), 1.07-1.00 (m, 2H), 0.96 (d, J = 6.7 Hz, 3H), 0.85 (d, J = 6.7 Hz, 3H); |
| 132 | 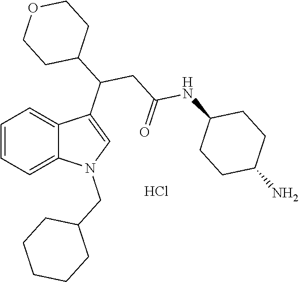 [SMILES: N[C@H]1CC[C@H](NC(=O)CC(c2cn(CC3CCCCC3)c3ccccc23)C2CCOCC2)CC1 ; Cl] | ¹H NMR (400 MHz, Methanol-d₄): δ 7.57 (d, J = 7.9 Hz, 1H), 7.30 (d, J = 8.2 Hz, 1H), 7.13-7.08 (m, 1H), 7.02-6.94 (m, 2H), 3.97-3.91 (m, 3H), 3.86-3.81 (m, 1H), 3.27-3.20 (m, 2H), 2.94-2.78 (m, 2H), 2.74-2.67 (m, 1H), 2.55-2.48 (m, 2H), 1.98-1.88 (m, 2H), 1.87-1.76 (m, 4H), 1.75-1.64 (m, 3H), 1.63-1.55 (m, 2H), 1.44-1.31 (m, 4H), 1.30-1.17 (m, 5H), 1.16-0.97 (m, 3H), 0.89-0.77 (m, 1H); |
| 133 | 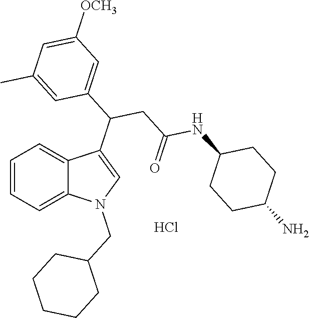 [SMILES: COc1cc(C)cc(C(CC(=O)N[C@H]2CC[C@H](N)CC2)c2cn(CC3CCCCC3)c3ccccc23)c1 ; Cl] | ¹H NMR (400 MHz, DMSO-d6): δ 7.75 (d, J = 7.6 Hz, 1H), 7.67 (bs, 3H), 7.36 (d, J = 8.4 Hz, 1H), 7.30 (d, J = 8.0 Hz, 1H), 7.25 (s, 1H), 7.02 (dt, J = 8.0, 1.2 Hz, 1H), 6.86 (dt, J = 7.6, 0.8 Hz, 1H), 6.64 (s, 1H), 6.58 (s, 1H), 6.50 (s, 1H), 4.45 (t, J = 8.0 Hz, 1H), 3.94 (d, J = 7.2 Hz, 2H), 3.4 (s, 3H), 3.39-3.34 (m, 1H), 2.93-2.79 (m, 1H), 2.67-2.65 (m, 1H), 2.64-2.62 (m, 1H), 2.18 (s, 3H), 1.90-1.84 (m, 3H), 1.78-1.48 (m, 7H), 1.29-1.23 (m, 2H), 1.12-0.96 (m, 7H); |
| 134 | 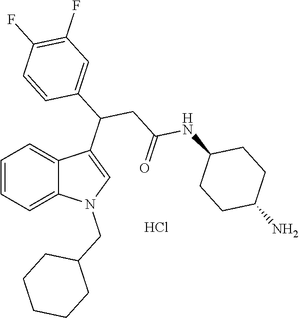 [SMILES: N[C@H]1CC[C@H](NC(=O)CC(c2ccc(F)c(F)c2)c2cn(CC3CCCCC3)c3ccccc23)CC1 ; Cl] | ¹H NMR (400 MHz, DMSO-d₆): δ 7.78 (d, J = 8.0 Hz, 1H), 7.69 (bs, 3H), 7.29 (d, J = 8.0 Hz, 1H), 7.85-7.22 (m, 4H), 7.08-7.04 (m, 2H), 6.88 (t, J = 7.2 Hz, 1H), 4.63 (t, J = 8.0 Hz, 1H), 3.32-3.31 (m, 1H), 3.94 (dd, J = 7.2, 2.0 Hz, 2H), 2.94-2.74 (m, 2H), 2.72-2.69 (m, 1H), 1.90-1.83 (m, 3H), 1.76-1.48 (m, 7H), 1.33-1.24 (m, 2H), 1.23-0.97 (m, 7H); |
| 135 | 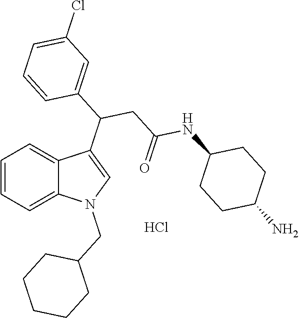 [SMILES: N[C@H]1CC[C@H](NC(=O)CC(c2cccc(Cl)c2)c2cn(CC3CCCCC3)c3ccccc23)CC1 ; Cl] | ¹H NMR (400 MHz; DMSO-d₆): δ 7.97-7.76 (m, 4H), 7.39 (d, J = 8.2 Hz, 1H), 7.33-7.27 (m, 2H), 7.27-7.21 (m, 3H), 7.20-7.14 (m, 1H), 7.09-7.02 (m, 1H), 6.93-6.86 (m, 1H), 4.66 (t, J = 7.8 Hz, 1H), 3.96 (d, J = 6.9 Hz, 2H), 3.42-3.34 (m, 1H), 2.96-2.80 (m, 2H), 2.77-2.68 (m, 1H), 1.95-1.82 (m, 2H), 1.82-1.44 (m, 8H), 1.37-0.90 (m, 9H). |
| 136 | 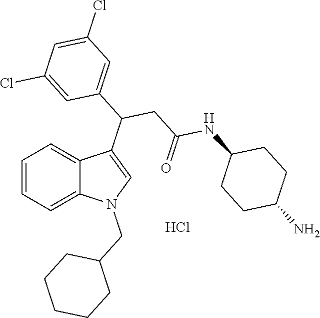 [SMILES: N[C@H]1CC[C@H](NC(=O)CC(c2cc(Cl)cc(Cl)c2)c2cn(CC3CCCCC3)c3ccccc23)CC1 ; Cl] | ¹H NMR 400 MHz; DMSO-d₆): δ 7.89-7.62 (m, 4H), 7.41 (d, J = 8.2 Hz, 1H), 7.37-7.31 (m, 3H), 7.26 (d, J = 2.3 Hz, 2H), 7.11-7.04 (m, 1H), 6.95-6.88 (m, 1H), 4.67 (t, J = 8.2 Hz, 1H), 4.03-3.91 (m, 2H), 3.43-3.34 (m, 1H), 2.99-2.72 (m, 3H), 1.93-1.82 (m, 2H), 1.82-1.43 (m, 8H), 1.37-1.22 (m, 2H), 1.21-0.92 (m, 7H). |
| 137 | 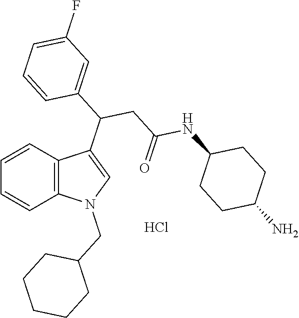 [SMILES: N[C@H]1CC[C@H](NC(=O)CC(c2cccc(F)c2)c2cn(CC3CCCCC3)c3ccccc23)CC1 ; Cl] | ¹H NMR (400 MHz; DMSO-d₆): δ 7.92-7.67 (m, 4H), 7.38 (d, J = 8.2 Hz, 1H), 7.34-7.21 (m, 3H), 7.13-6.98 (m, 3H), 6.97-6.85 (m, 2H), 4.67 (t, J = 7.8 Hz, 1H), 4.04-3.90 (m, 2H), 3.42-3.32 (m, 1H), 2.98-2.79 (m, 2H), 2.77-2.68 (m, 1H), 1.93-1.44 (m, 10H), 1.36-1.22 (m, 2H), 1.20-0.91 (m, 7H). |
| 138 | 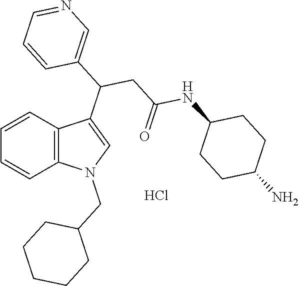 [SMILES: N[C@H]1CC[C@H](NC(=O)CC(c2cccnc2)c2cn(CC3CCCCC3)c3ccccc23)CC1 ; Cl] | ¹H NMR (400 MHz; DMSO-d₆): δ 8.78 (bs, 1H), 8.63 (d, J = 5.0 Hz, 1H), 8.24 (d, J = 8.2 Hz, 1H), 8.03-7.86 (m, 4H), 7.78 (t, J = 6.9 Hz, 1H), 7.42 (d, J = 8.2 Hz, 1H), 7.40-7.34 (m, 2H), 7.13-7.04 (m, 1H), 6.92 (t, J = 7.3 Hz, 1H), 4.86 (t, J = 7.8 Hz, 1H), 4.04-3.90 (m, 2H), 3.39-3.29 (m, 1H), 2.99- 2.83 (m, 2H), 1.94-1.83 (m, 2H), 1.82-1.46 (m, 8H), 1.37- 1.24 (m, 2H), 1.21-0.91 (m, 8H). |
| 139 | 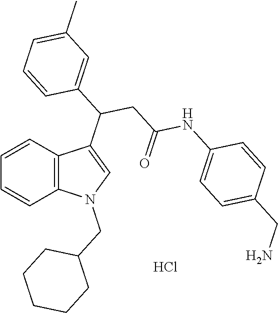 [SMILES: Cc1cccc(C(CC(=O)Nc2ccc(CN)cc2)c2cn(CC3CCCCC3)c3ccccc23)c1 ; Cl] | ¹H NMR (400 MHz, DMSO-d₆): δ 10.09 (s, 1H), 8.15-7.93 (m, 3H), 7.54 (d, J = 8.6 Hz, 2H), 7.39-7.29 (m, 5H), 7.16- 7.09 (m, 3H), 7.05 (t, J = 7,7 Hz, 1H), 6.96-6.87 (m, 2H), 4.72 (t, J = 7.9 Hz, 1H), 4.04-3.89 (m, 4H), 3.20-3.10 (m, 1H), 3.01-2.91 (m, 1H), 2.22 (s, 3H), 1.78-1.66 (m, 1H), 1.61-1.42 (m, 5H), 1.12-1.00 (m, 3H), 0.99-0.83 (m, 2H); |
| 140 | 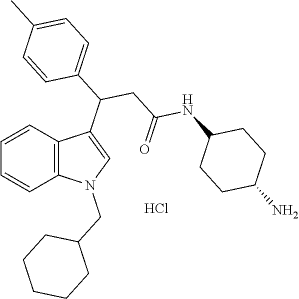 | ¹H NMR (400 MHz, Methanol-d₄): δ 7.33-7.27 (m, 2H), 7.16 (d, J = 8.4 Hz, 2H), 7.10-7.02 (m, 4H), 6.88 (t, J = 7.6 Hz, 1H), 4.68 (t, J = 7.9 Hz, 1H), 3.94 (d, J = 7.1 Hz, 2H), 2.99-2.91 (m, 2H), 2.83-2.76 (m, 1H), 2.26 (s, 3H), 1.99- 1.91 (m, 2H), 1.88-1.80 (m, 1H), 1.78-1.65 (m, 5H), 1.64- 1.55 (m, 2H), 1.47-1.31 (m, 3H), 1.24-1.17 (m, 3H), 1.16- 0.97 (m, 4H); |
| 142 | 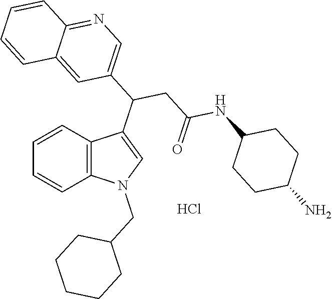 | ¹H NMR (400 MHz, DMSO-d₆): δ 8.91 (s, 1H), 8.38 (bs, 1H), 8.01-7.91 (m, 3H), 7.83-7.75 (m, 4H), 7.62 (t, J = 7.4 Hz, 1H), 7.42-7.35 (m, 3H), 7.04 (t, J = 7.2 Hz, 1H), 6.85 (t, J = 8.0 Hz, 1H), 4.90 (t, J = 8.0 Hz, 1H), 3.97 (dd, J = 7.2, 3.2 Hz, 2H), 3.36-3.31 (m, 1H), 3.04-2.89 (m, 3H), 1.90-1.77 (m, 4H), 1.65-1.61 (m, 4H), 1.53-1.51 (m, 2H), 1.36-1.23 (m, 2H), 1.20-1.18 (m, 4H), 1.14- 0.94 (m, 3H); |
| 143 | 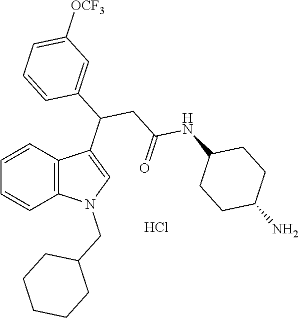 | ¹H NMR (400 MHz, DMSO-d₆): δ 7.81 (s, 1H), 7.79 (bs, 3H), 7.40-7.29 (m, 5H), 7.18 (s, 1H), 7.18-7.04 (m, 2H), 6.91-6.87 (m, 1H), 4.69 (t, J = 8.0 Hz, 1H), 3.95 (dd, J = 7.0, 2.4 Hz, 2H), 2.95-2.83 (m, 2H), 2.79-2.73 (m, 1H), 1.85-1.79 (m, 2H), 1.78-1.74 (m, 1H), 1.64-1.60 (m, 4H), 1.54-1.47 (m, 2H), 1.33-1.22 (m, 3H), 1.17-1.11 (m, 5H), 1.04-0.95 (m, 3H); |
| 144 | 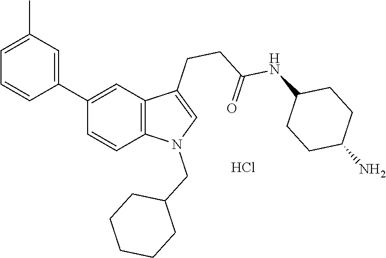 | ¹H NMR (400 MHz, DMSO-d₆): δ 7.91 (bs, 3H), 7.74-7.71 (m, 2H), 7.51-7.42 (m, 3H), 7.41-7.27 (m, 2H), 7.13-7.05 (m, 2H), 3.94 (d, J = 7.0 Hz, 2H), 3.51-3.43 (m, 1H), 3.00- 2.89 (m, 3H), 2.43-2.37 (m, 5H), 1.95-1.85 (m, 2H), 1.81- 1.71 (m, 3H), 1.70-1.57 (m, 3H), 1.56-1.46 (m, 2H), 1.41- 1.27 (m, 2H), 1.19-1.06 (m, 5H), 1.04-0.91 (m, 2H). |
| 145 | 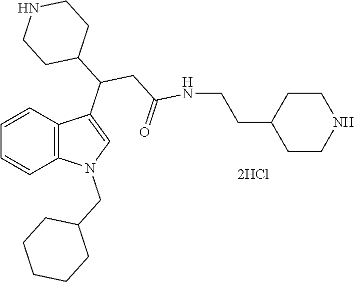 [SMILES: O=C(CC(c1cn(CC2CCCCC2)c2ccccc12)C1CCNCC1)NCCC1CCNCC1 ; "2HCl"] | ¹H NMR (400 MHz, DMSO-d₆): δ 8.37 (bs, 4H), 7.71(t, J = 5.6 Hz, 1H), 7.52 (d, J = 8.0 Hz, 1H), 7.38 (d, J = 8.0 Hz, 1H), 7.11 (s, 1H), 7.06 (dt, J = 8.0, 0.8 Hz, 1H), 6.96 (t, J = 7.0 Hz, 1H), 3.87 (h, J = 8.0 Hz, 2H), 3.31-3.30 (m, 1H), 3.21-3.09 (m, 4H), 2.80-2.94 (m, 3H), 2.77-2.66 (m, 3H), 1.87-1.74 (m, 3H), 1.73-1.50 (m, 9H), 1.38-1.25 (m, 3H), 1.12 (bs, 8H), 1.0-0.94 (m, 2H), |
| 146 | 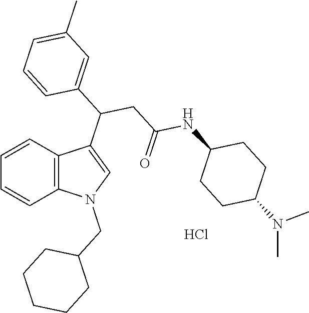 [SMILES: Cc1cccc(C(CC(=O)N[C@H]2CC[C@H](N(C)C)CC2)c2cn(CC3CCCCC3)c3ccccc23)c1 ; Cl] | ¹H NMR (400 MHz, Methanol-d₄): δ 7.28 (d, J = 8.8 Hz, 2H), 7.13-7.05 (m, 5H), 6.95 (d, J = 6.8 Hz, 1H), 6.86 (t, J = 7.2 Hz, 1H), 4.66 (t, J = 8.0 Hz, 1H), 3.94 (d, J = 7.2 Hz, 2H), 3.53-3.49 (m, 1H), 3.08-3.13 (m, 1H), 2.98-2.93 (m, 1H), 2.77 (s, 6H), 2.25 (s, 3H), 2.02-1.98 (m, 2H), 1.85- 1.48 (m, 10H), 1.38-1.21 (m, 5H), 1.09-1.01 (m, 3H);. |
| 150 | 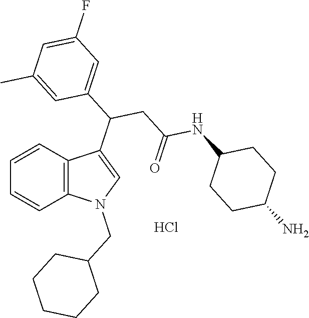 | ¹H NMR (400 MHz, Methanol-d₄): δ 7.31 (d, J = 9.0 Hz, 2H), 7.12-7.06 (m, 2H), 6.94-6.89 (m, 2H), 6.76 (d, J = 9.9 Hz, 1H), 6.70 (d, J = 9.7 Hz, 1H), 4.70 (t, J = 8.0 Hz, 1H), 3.97 (d, J = 7.2 Hz, 2H), 3.54-3.46 (m, 1H), 3.04-2.91 (m, 2H), 2.83-2.76 (m, 1H), 2.27 (s, 3H), 2.01-1.93 (m, 2H), 1.90-1.82 (m, 1H), 1.81-1.70 (m, 4H), 1.69-1.65 (m, 1H), 1.64-1.55 (m, 2H), 1.46-1.34 (m, 2H), 1.30-1.17 (m, 4H), 1.15-0.99 (m, 3H); |
| 151 | 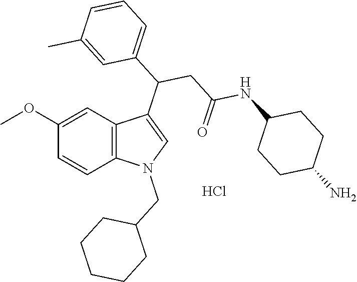 | ¹H NMR (400 MHz, Methanol-d₄): δ 7.74 (d, J = 7.6 Hz, 1H), 7.68 (bs, 3H), 7.26 (d, J = 8.8 Hz, 1H), 7.20 (s, 1H), 7.13-7.03 (m, 3H), 6.93 (d, J = 7.3 Hz, 1H), 6.77-6.73 (m, 1H), 6.72-6.67 (m, 1H), 4.55 (t, J = 7.8 Hz, 1H), 3.90 (d, J = 7.0 Hz, 2H), 3.65 (s, 3H), 2.98-2.89 (m, 1H), 2.87-2.79 (m, 1H), 2.69-2.61 (m, 1H), 2.22 (s, 3H), 1.89-1.82 (m, 2H), 1.79-1.70 (m, 1H), 1.69-1.58 (m, 5H), 1.56-1.45 (m, 2H), 1.35-1.22 (m, 3H), 1.19-1.06 (m, 5H), 1.01-0.92 (m, 2H); |
| 153 | 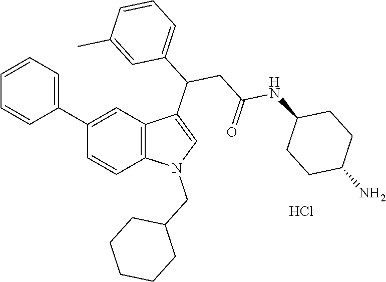 [SMILES: Cc1cccc(C(CC(=O)N[C@H]2CC[C@H](N)CC2)c2cn(CC3CCCCC3)c3ccc(-c4ccccc4)cc23)c1 ; Cl] | ¹H NMR (400 MHz, DMSO-d₆): δ 7.80 (bs, 4H), 7.55-7.50 (m, 3H), 7.47 (d, J = 7.4 Hz, 1H), 7.41 (t, J = 7.4 Hz, 2H), 7.37-7.32 (m, 1H), 7.30-7.23 (m, 2H), 7.14-7.03 (m, 3H), 6.94-6.87 (m, 1H), 4.68 (t, J = 7.9 Hz, 1H), 3.98 (d, J = 7.2 Hz, 2H), 3.70-3.53 (m, 1H), 3.40-3.32 (m, 1H), 3.16-3.06 (m, 1H), 2.99-2.79 (m, 2H), 2.76-2.61 (m, 1H), 2.21 (s, 3H), 1.89-1.81 (m, 2H), 1.69-1.60 (m, 4H), 1.56-1.50 (m, 1H), 1.30-1.23 (m, 7H), 1.17-1.07 (m, 3H). |
| 154 | 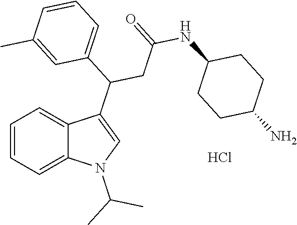 [SMILES: Cc1cccc(C(CC(=O)N[C@H]2CC[C@H](N)CC2)c2cn(C(C)C)c3ccccc23)c1 ; Cl] | ¹H NMR (400 MHz, DMSO-d₆): δ 7.89-7.75 (m, 4H), 7.46- 7.39 (m, 2H), 7.29 (d, J = 7.6 Hz, 1H), 7.14-7.0 (m, 4H), 6.95-6.85 (m, 2H), 4.77-4.65 (m, 2H), 3.46-3.34 (m, 1H), 2.98-2.82 (m, 2H), 2.71-2.62 (m, 1H), 2.22 (s, 3H), 1.93-1.82 (m, 2H), 1.71-1.59 (m, 2H), 1.45 (dd, J = 19.2, 6.4 Hz, 6H), 1.38-1.21 (m, 3H), 1.20-1.02 (m, 3H); |
| 155 | 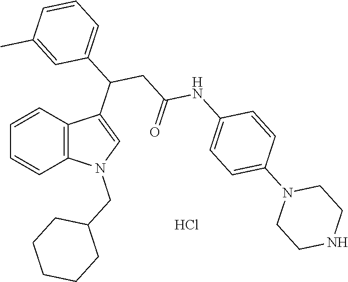 [SMILES: Cc1cccc(C(CC(=O)Nc2ccc(N3CCNCC3)cc2)c2cn(CC3CCCCC3)c3ccccc23)c1 ; Cl] | ¹H NMR (400 MHz, DMSO-d₆): δ 9.69 (s, 1H), 7.40-7.30 (m, 6H), 7.14-7.10 (m, 3H), 7.08-7.01 (m, 2H), 6.96- 6.86 (m, 3H), 6.80 (d, J = 8.0 Hz, 1H), 4.71 (t, J = 7.6 Hz, 1H), 4.02-3.87 (m, 2H), 3.13-2.97 (m, 5H), 2.91-2.83 (m, 1H), 2.22 (s, 3H), 1.54-1.37 (m, 11H), 1.07-0.78 (m, 4H); |
| 157 | 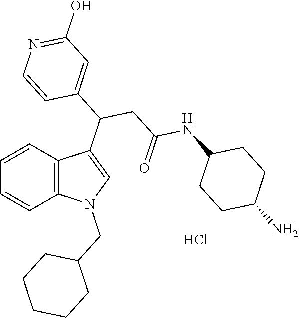 | ¹H NMR (400 MHz, DMSO-d₆): δ 7.96-7.77 (m, 5H), 7.39 (t, J = 8.8 Hz, 2H), 7.26 (s, 1H), 7.19 (d, J = 6.8 Hz, 1H), 7.09 (t, J = 7.6 Hz, 1H), 6.94 (t, J = 7.3 Hz, 1H), 6.20 (s, 1H), 6.06-6.00 (m, 1H), 4.44 (t, J = 7.8 Hz, 1H), 3.99-3.93 (m, 2H), 2.99-2.90 (m, 1H), 2.85-2.65 (m, 3H), 1.92-1.85 (m, 2H), 1.79-1.70 (m, 2H), 1.69-1.58 (m, 4H), 1.57-1.47 (m, 2H), 1.37-1.28 (m, 2H), 1.18-1.08 (m, 5H), 1.04-0.94 (m, 2H); |
| 158 | 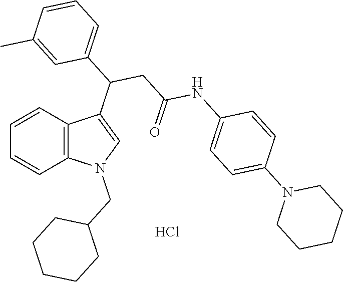 [SMILES: Cc1cccc(C(CC(=O)Nc2ccc(N3CCCCC3)cc2)c2cn(CC3CCCCC3)c3ccccc23)c1 ; Cl] | ¹H NMR (400 MHz, DMSO-d₆): δ 7.61 (bs, 3H), 7.40- 7.28 (m, 4H), 7.17-7.01 (m, 5H), 6.98-6.85 (m, 3H), 4.72 (t, J = 8.8 Hz, 1H), 4.08-3.82 (m, 2H), 3.22-3.07 (m, 1H), 3.0-2.88 (m, 1H), 2.22 (s, 3H), 1.92-1.62 (m, 5H), 1.60-1.34 (m, 8H), 1.30-1.20 (m, 2H), 1.05-0.94 (m, 4H), 0.93-0.82-(m, 2H); |
| 160 | 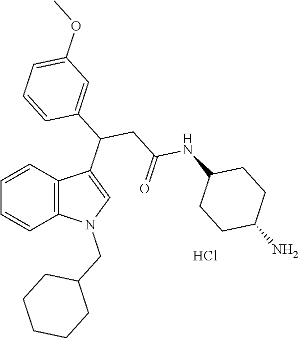 | ¹H NMR (400 MHz, DMSO-d₆): δ 7.83-7.73 (m, 4H), 7.38 (d, J = 8.3 Hz, 1H), 7.31 (d, J = 8.0 Hz, 1H), 7.26 (s, 1H), 7.13 (t, J = 7.9 Hz, 1H), 7.05 (t, J = 7.2 Hz, 1H), 6.90-6.81 (m, 2H), 6.80-6.77 (m, 1H), 6.71-6.66 (m, 1H), 4.62 (t, J = 7.9 Hz, 1H), 3.95 (d, J = 7.1 Hz, 2H), 3.67 (s, 3H), 3.41- 3.35 (m, 1H), 2.98-2.90 (m, 1H), 2.89-2.81 (m, 1H), 2.72- 2.65 (m, 1H), 1.90-1.82 (m, 2H), 1.81-1.72 (m, 1H), 1.70- 1.59 (m, 5H), 1.58-1.47 (m, 2H), 1.34-1.22 (m, 2H), 1.19- 1.07 (m, 5H), 1.06-0.95 (m, 2H); |
| 161 | 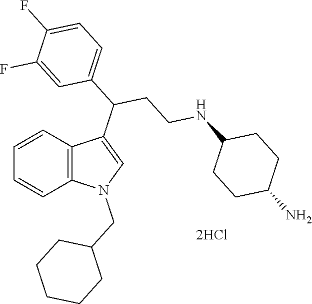 | ¹H NMR (400 MHz, DMSO-d₆): δ 9.25 (bs, 2H), 7.95 (bs, 3H), 7.43-7.30 (m, 5H), 7.18 (bs, 1H), 7.08 (dt, J = 8.0, 0.8 Hz, 1H), 6.91 (t, J = 7.6 Hz, 1H), 4.36 (t, J = 7.6 Hz, 1H), 3.98 (d, J = 7.6 Hz, 2H), 3.07-3.04 (m, 3H), 2.94- 2.91 (m, 1H), 2.50-2.49 (m, 1H), 2.33-2.33 (m, 1H), 1.95- 1.81 (m, 4H), 1.80-1.77 (m, 1H), 1.64-1.60 (m, 3H), 1.52-1.49 (m, 2H), 1.40-1.29 (m, 4H), 1.17-1.11 (m, 3H), 1.02-0.97 (m, 2H); |
| 162 | 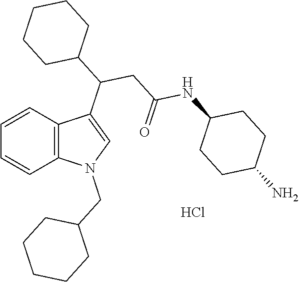 [SMILES: N[C@H]1CC[C@H](NC(=O)CC(c2cn(CC3CCCCC3)c3ccccc23)C2CCCCC2)CC1 ; Cl] | ¹H NMR (400 MHz, DMSO-d₆): δ 7.73 (s, 3H), 7.58 (d, J = 7.5 Hz, 1H), 7.48 (d, J = 7.9 Hz, 1H), 7.36 (d, J = 8.1 Hz, 1H), 7.02-6.91 (m, 3H), 3.92 (d, J = 7.0 Hz, 2H), 2.96-2.85 (m, 1H), 2.47-2.30 (m, 4H), 1.90-1.70 (m, 5H), 1.68-1.59 (m, 6H), 1.55-1.44 (m, 5H), 1.33-1.21 (m, 2H), 1.17-1.07 (m, 6H), 1.01-0.80 (m, 6H); |
| 163 | 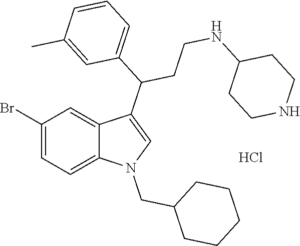 [SMILES: Cc1cccc(C(CCNC2CCNCC2)c2cn(CC3CCCCC3)c3ccc(Br)cc23)c1 ; Cl] | ¹H NMR (400 MHz, DMSO-d₆): δ 9.31 (bs, 2H), 8.87 (bs, 2H), 7.54 (d, J = 1.8 Hz, 1H), 7.42 (d, J = 7.4 Hz, 2H), 7.21-7.11 (m, 4H), 6.99 (d, J = 7.2 Hz, 1H), 4.29 (t, J = 7.5 Hz, 1H), 3.99 (d, J = 6.6 Hz, 2H), 3.35, 3.28 (m, 2H), 3.03- 2.69 (m, 4H), 2.44-2.27 (m, 2H), 2.25 (s, 3H), 2.11-2.08 (m, 2H), 1.79-1.74 (m, 3H), 1.71-1.54 (m, 3H), 1.51-1.42 (m, 2H), 1.40-0.75 (m, 6H). |
| 164 | 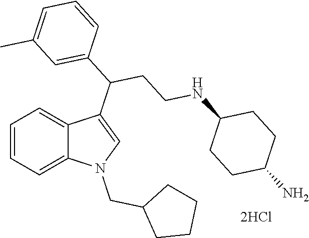 [SMILES: Cc1cccc(C(CCN[C@H]2CC[C@H](N)CC2)c2cn(CC3CCCC3)c3ccccc23)c1 ; "2HCl"] | ¹H NMR (400 MHz, DMSO-d₆) δ 9.08 (bs, 2H), 8.06 (bs, 3H), 7.48-7.34 (m, 3H), 7.20-7.02 (m, 4H), 7.01-6.86 (m, 2H), 4.26 (t, J = 7.6 Hz, 1H), 4.06 (d, J = 7.6 Hz, 2H), 3.03- 2.72 (m, 4H), 2.43-2.27 (m, 2H), 2.24 (s, 3H), 2.09-1.92 (m, 4H), 1.70-1.17 (m, 13H). |
| 165 | 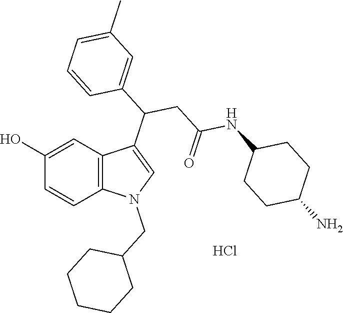 | ¹H NMR (400 MHz, DMSO-d₆): δ 8.53 (s, 1H), 7.78-7.69 (m, 4H), 7.18-7.07 (m, 3H), 7.04-6.97 (m, 2H), 6.92 (d, J = 7.4 Hz, 1H), 6.60-6.53 (m, 2H), 4.48 (t, J = 7.8 Hz, 1H), 3.86 (d, J = 7.0 Hz, 2H), 3.00-2.88 (m, 1H), 2.83-2.75 (m, 1H), 2.64-2.57 (m, 1H), 2.22 (s, 3H), 1.90-1.81 (m, 2H), 1.75-1.59 (m, 6H), 1.56-1.47 (m, 2H), 1.36-1.23 (m, 3H), 1.19-1.09 (m, 4H), 1.06-0.91 (m, 3H); |
| 166 | 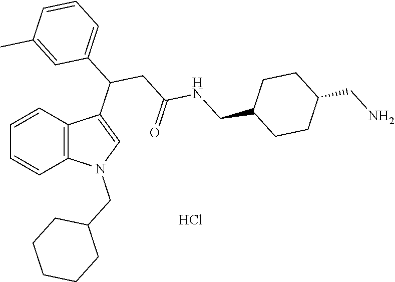 | ¹H NMR (400 MHz, DMSO-d₆): δ 7.76 (t, J = 5.6 Hz, 1H), 7.70 (bs, 3H), 7.36 (d, J = 8.4 Hz, 1H), 7.29 (d, J = 8.0 Hz, 1H), 7.24 (s, 1H), 7.11-7.02 (m, 4H), 6.98 (d, J = 7.2 Hz, 1H), 6.85 (dt, J = 8.0, 0.8 Hz, 1H), 4.57 (t, J = 8.0 Hz, 1H), 4.0-3.93 (m, 2H), 2.92-2.82 (m, 2H), 2.76-2.76 (m, 4H), 2.21 (s, 3H), 1.82-1.78 (m, 1H), 1.77-1.61 (m, 5H), 1.53- 1.36 (m, 5H), 1.11 (bs, 4H), 1.01-0.95 (m, 2H), 0.85- 0.62 (m, 4H); |
| 167 | 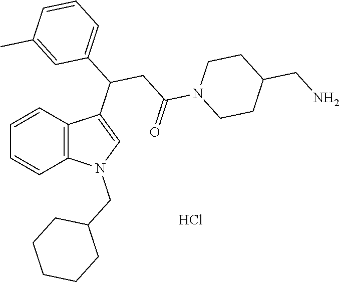 [SMILES: Cc1cccc(C(CC(=O)N2CCC(CN)CC2)c2cn(CC3CCCCC3)c3ccccc23)c1 ; Cl] | ¹H NMR (400 MHz, DMSO-d₆): δ 7.66 (bs, 3H), 7.38-7.27 (m, 3H), 7.11-7.09 (m, 3H), 7.02 (t, J = 7.2 Hz, 1H), 6.92- 6.86 (m, 2H), 4.61 (t, J = 7.6 Hz, 1H), 4.26 (t, J = 13.4 Hz, 1H), 4.01 (bs, 1H), 3.94 (d, J = 7.2 Hz, 2H), 3.11-3.01 (m, 2H), 2.89-2.78 (m, 1H), 2.67-2.4 (m, 2H), 2.49-2.31 (m, 1H), 2.21 (s, 3H), 1.79-1.49 (m, 9H), 1.11 (bs, 3H), 0.98-0.58 (m, 4H); |
| 168 | 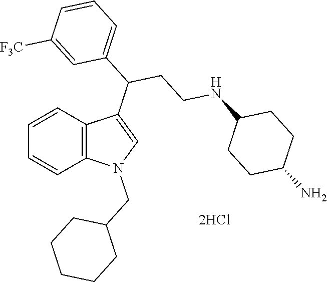 | ¹H NMR (400 MHz, DMSO-d₆): δ 8.98 (bs, 2H), 7.96 (bs, 3H), 7.68-7.64 (m, 2H), 7.54-7.37 (m, 5H), 7.06 (t, J = 7.5 Hz, 1H), 6.90 (t, J = 7.5 Hz, 1H), 4.48 (t, J = 7..5 Hz, 1H), 3.99 (d, J = 6.6 Hz, 2H), 2.95-2.82 (m, 4H), 2.39- 2.35 (m, 1H), 2.04-1.99 (m, 4H), 1.83-1.79 (m, 1H), 1.63 (bs, 4H), 1.37-1.25 (m, 5H), 1.13-0.82 (m, 6H). |
| 169 | 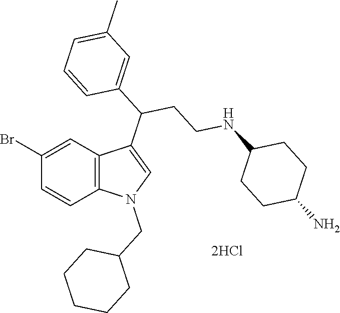 | ¹H NMR (400 MHz, DMSO-d₆): δ 7.53 (d, J = 1.86 Hz, 1H), 7.40-7.48 (m, 2H), 7.31 (bs, 3H), 7.28-7.09 (m, 6H), 6.99 (d, J = 7.3 Hz, 1H), 4.28 (t, J = 7.8 Hz, 1H), 3.99 (d, J = 7.2 Hz, 2H), 3.05 (d, J = 7.3 Hz, 1H), 2.99-2.83 (m, 3H), 2.81-2.71 (m, 1H), 2.37-2.27 (m, 1H), 2.24 (s, 3H), 2.11- 1.90 (m, 4H), 1.85-1.73 (m, 1H), 1.70-1.55 (m, 3H), 1.48- 1.45 (m, 2H), 1.41-1.30 (m, 3H), 1.20-1.10 (m, 4H), 1.03- 0.91 (m, 2H). |
| 171 | 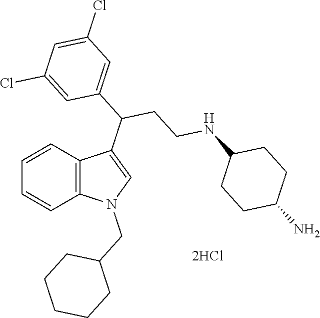 | ¹H NMR (400 MHz, DMSO-d₆): δ 9.51-8.86 (m, 2H), 8.09 (bs, 3H), 7.52 (s, 1H), 7.47-7.28 (m, 5H), 7.09 (t, J = 7.5 Hz, 1H), 6.95 (t, J = 7.5 Hz, 1H), 4.54-4.42 (m, 1H), 4.00 (d, J = 6.2 Hz, 2H), 3.06-2.72 (m, 4H), 2.42-2.27 (m, 1H), 2.17-1.91 (m, 4H), 1.87-1.73 (m, 1H), 1.71-1.22 (m, 10H), 1.20-0.90 (m, 5H). |
| 172 | 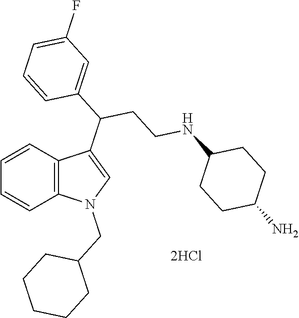 | ¹H NMR (400 MHz, DMSO-d₆): δ 9.21-8.67 (m, 2H), 7.92 (bs, 3H), 7.46-7.27 (m, 4H), 7.23-7.11 (m, 2H), 7.08 (t, J = 7.6 Hz, 1H), 7.03-6.95 (m, 1H), 6.92 (t, J = 7.1 Hz, 1H), 4.37 (t, J = 7.1 Hz, 1H), 4.04-3.92 (m, 2H), 3.03-2.72 (m, 4H), 2.36-2.35 (m, 1H), 2.09-1.91 (m, 4H), 1.85-1.72 (m, 1H), 1.71-1.56 (m, 3H), 1.55-1.46 (m, 2H), 1.45-1.21 (m, 5H), 1.18-1.08 (m, 3H), 1.05-0.90 (m, 2H). |
| 173 | 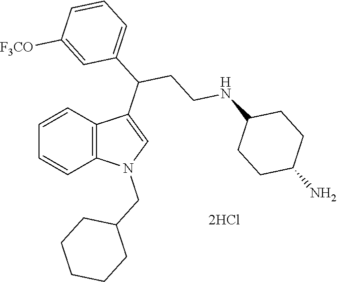 | ¹H NMR (400 MHz, DMSO-d₆): δ 9.02 (bs, 2H), 7.91 (bs, 3H), 7.44-7.38 (m, 5H), 7.28 (s, 1H), 7.15 (d, J = 7.2 Hz, 1H), 7.07 (t, J = 7.2 Hz, 1H), 6.90 (t, J = 7.2 Hz, 1H), 4.41 (t, J = 7.2 Hz, 1H), 3.98 (d, J = 6.8 hz, 2H), 2.94-2.81 (m, 4H), 2.32 (t, J = 2.0 Hz, 1H), 1.98-1.80 (m, 4H), 1.79- 1.74 (m, 1H), 1.60-1.49 (m, 5H), 1.34-1.23 (m, 5H), 1.12 (bs, 3H), 1.11-0.96 (m, 2H); |
| 174 | 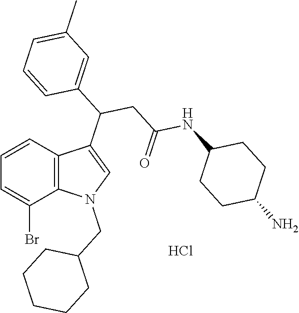 | ¹H NMR (400 MHz, DMSO-d₆): δ 7.86-7.82 (m, 4H), 7.35- 7.23 (m, 3H), 7.13-7.07 (m, 1H), 7.05-7.00 (m, 2H), 6.93 (d, J = 7.4 Hz, 1H), 6.81 (t, J = 7.8 Hz, 1H), 4.59 (t, J = 8.0 Hz, 1H), 4.37-4.20 (m, 2H), 3.00-2.89 (m, 1H), 2.88-2.80 (m, 1H), 2.69-2.60 (m, 1H), 2.20 (s, 3H), 1.90-1.75 (m, 3H), 1.72-1.60 (m, 5H), 1.52-1.45 (m, 2H), 1.35-1.22 (m, 3H), 1.19-0.98 (m, 7H); |
| 175 | 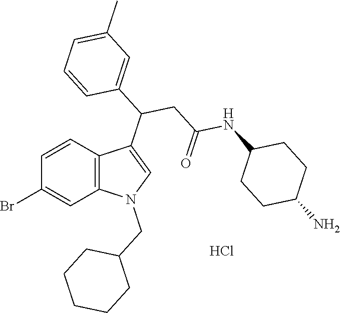 | ¹H NMR (400 MHz, DMSO-d₆): δ 7.76 (d, J = 7.2 Hz, 1H), 7.66 (d, J = 1.6 Hz, 1H), 7.46 (bs, 3H), 7.28 (s, 1H), 7.21 (d, J = 8.4 Hz, 1H), 7.10 (t, J = 7.3 Hz, 1H), 7.05-6.97 (m, 3H), 6.93 (d, J = 7.3 Hz, 1H), 4.58 (t, J = 7.9 Hz, 1H), 3.97-3.94 (m, 2H), 3.01-2.73 (m, 2H), 2.68-2.59 (m, 1H), 2.20 (s, 3H), 1.91-1.79 (m, 2H), 1.78-1.56 (m, 6H), 1.56-1.42 (m, 2H), 1.40-0.78 (m, 10H). |
| 177 | 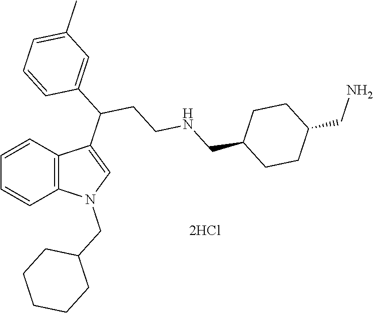 [SMILES: Cc1cccc(C(CCNC[C@H]2CC[C@H](CN)CC2)c2cn(CC3CCCCC3)c3ccccc23)c1 ; "2HCl"] | ¹H NMR (400 MHz, DMSO-d₆): δ 8.60 (bs, 3H), 7.80 (bs, 2H), 7.37 (t, J = 8.8 Hz, 2H), 7.32 (s, 1H), 7.16-7.05 (m, 4H), 6.96 (d, J = 6.8 Hz, 1H), 6.88 (t, J = 7.2 Hz, 1H), 4.20 (t, J = 7.6 Hz, 1H), 3.97 (d, J = 7.2 Hz, 2H), 2.78-2.74 (m, 1H), 2.73-2.71 (m, 3H), 2.66-2.62 (m, 2H), 2.33-2.27 (m, 1H), 2.24 (s, 3H), 1.79-1.75 (m, 5H), 1.65-1.49 (m, 7H), 1.15-1.11 (m, 3H), 1.02-0.85 (m, 7H); |
| 178 | 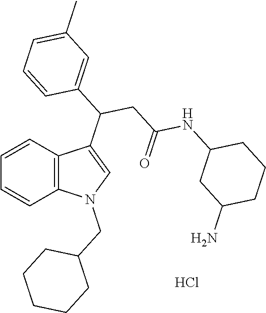 [SMILES: Cc1cccc(C(CC(=O)NC2CCCC(N)C2)c2cn(CC3CCCCC3)c3ccccc23)c1 ; Cl] | ¹H NMR (400 MHz, DMSO-d₆): δ 7.91-7.85 (m, 1H), 7.83 (bs, 3H), 7.36 (dd, J = 8.0, 2.4 Hz, 1H), 7.29-7.23 (m, 2H), 7.03-7.01 (m, 4H), 6.93-6.85 (m, 2H), 4.58 (dt, J = 7.6, 4.0 Hz, 1H), 3.99-3.90 (m, 2H), 3.48-3.30 (m, 1H), 2.98- 2.92 (m, 1H), 2.90-2.84 (m, 1H), 2.79-2.66 (m, 1H), 2.32 (s, 3H), 1.94-1.90 (m, 1H), 1.84-1.52 (m, 10H), 1.23-0.96 (m, 8H); |
| 179 | 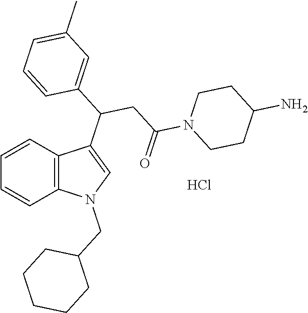 [SMILES: Cc1cccc(C(CC(=O)N2CCC(N)CC2)c2cn(CC3CCCCC3)c3ccccc23)c1 ; Cl] | ¹H NMR (400 MHz, DMSO-d₆): δ 8.20 (bs, 3H), 7.80- 7.67 (m, 3H), 7.55-7.45 (m, 3H), 7.45 (t, J = 7.6 Hz, 1H), 7.34-7.29 (m, 2H), 5.02 (q, J = 7.2 Hz, 1H), 4.68 (bs, 1H), 4.44 (t, J = 11.2 Hz, 1H), 4.32 (d, J = 7.2 Hz, 2H), 3.61- 3.33 (m, 4H), 2.63 (s, 3H), 2.28-2.17 (m, 3H), 2.08-2.03 (m, 3H), 1.94-1.91 (m, 2H), 1.85-1.82 (m, 2H), 1.68- 1.55 (m, 4H), 1.41-1.39 (m, 2H); |
| 180 | 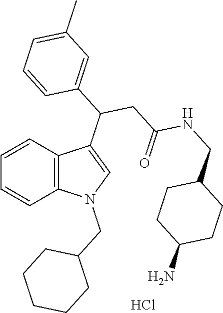 | ¹H NMR (400 MHz, DMSO-d₆): δ 7.80-7.70 (m, 4H), 7.40- 7.31 (m, 2H), 7.23 (s, 1H), 7.13-7.02 (m, 4H), 6.95-6.86 (m, 2H), 4.59 (t, J = 8.0 Hz, 1H), 4.02-3.89 (m, 2H), 2.94-2.81 (m, 2H), 2.80-2.72 (m, 2H), 2.22 (s, 3H), 1.83-1.73 (m, 3H), 1.70-1.54 (m, 4H), 1.53-1.39 (m, 4H), 1.16-1.06 (m, 6H), 1.03-0.95 (m, 2H), 0.81-0.64 (m, 2H). |
| 181 | 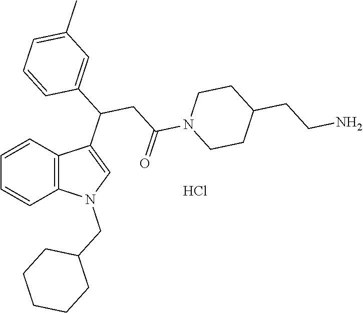 [SMILES: Cc1cccc(C(CC(=O)N2CCC(CCN)CC2)c2cn(CC3CCCCC3)c3ccccc23)c1 ; Cl] | ¹H NMR (400 MHz, DMSO-d₆): δ 7.68 (bs, 3H), 7.40-7.24 (m, 3H), 7.15-7.00 (m, 4H), 6.98-6.85 (m, 2H), 4.68-4.58 (m, 1H), 4.34 (t, J = 7.2 Hz, 1H) 4.01-3.90 (m, 3H), 3.17- 3.04 (m, 1H), 3.02-2.93 (m, 1H), 2.90-2.71 (m, 3H), 2.40- 2.30 (m, 1H), 2.22 (s, 3H), 1.83-1.71 (m, 1H), 1.70-1.59 (m, 3H), 1.58-1.47 (m, 4H), 1.46-1.30 (m, 3H), 1.19-1.10 (m, 3H), 1.04-0.94 (m, 2H), 0.85-0.39 (m, 2H); |
| 182 | 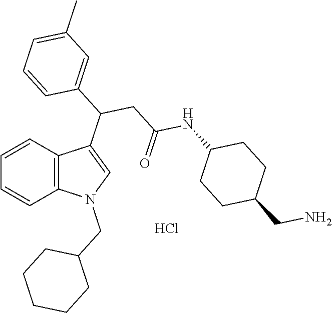 | ¹H NMR (400 MHz, DMSO-d₆): δ 7.77-7.60 (m, 4H), 7.37 (d, J = 8.7, 1H), 7.33-7.24 (m, 2H), 7.14-7.01 (m, 4H), 6.95- 6.83 (m, 2H), 4.60 (t, J = 7.6 Hz, 1H), 3.95 (d, J = 7.2 Hz, 2H), 3.41-3.35 (m, 1H), 2.89-2.79 (m, 1H), 2.69-2.60 (m, 3H), 2.21 (s, 3H), 1.82-1.60 (m, 8H), 1.57-1.37 (m, 3H), 1.19-1.08 (m, 3H), 1.07-0.91 (m, 6H); |
| 183 | 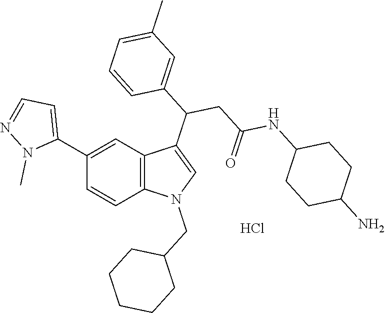 [SMILES: Cc1cccc(C(CC(=O)NC2CCC(N)CC2)c2cn(CC3CCCCC3)c3ccc(-c4ccnn4C)cc23)c1 ; Cl] | ¹H NMR (400 MHz, DMSO-d₆): δ 7.87 (bs, 3H), 7.80 (d, J = 7.6 Hz, 1H), 7.49 (d, J = 8.7 Hz, 1H), 7.42 (d, J = 1.7 Hz, 1H), 7.40-7.31 (m, 2H), 7.18-7.14 (m, 1H), 7.14-7.04 (m, 3H), 7.09-7.06 (m, 1H), 6.93 (d, J = 6.9 Hz, 1H), 4.64 (t, J = 7.7 Hz, 1H), 3.99 (d, J = 6.7 Hz, 2H), 3.70 (s, 3H), 3.40- 3.29 (m, 1H), 2.96-2.81 (m, 2H), 2.74-2.65 (m, 1H), 2.20 (s, 3H), 1.91-1.82 (m, 2H), 1.81-1.73 (m, 1H), 1.70-1.50 (m, 7H), 1.35-1.23 (m, 2H), 1.19-0.95 (m, 7H). |
| 186 | 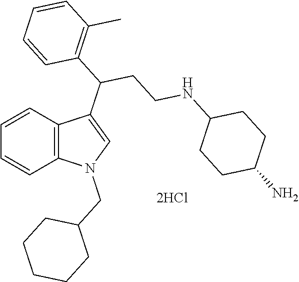 | ¹H NMR (400 MHz; DMSO-d₆): δ 9.04 (bs, 2H), 8.00 (bs, 3H), 7.40 (d, J = 8.1 Hz, 1H), 7.33 (d, J = 8.1 Hz, 1H), 7.29- 7.20 (m, 2H), 7.18-7.7.03 (m, 4H), 6.90 (t, J = 7.4 Hz, 1H), 4.45 (t, J = 7.0 Hz, 1H), 4.03-3.91 (m, 2H), 3.03-2.80 (m, 4H), 2.38 (s, 3H), 2.34-2.25 (m, 1H), 2.08-1.92 (m, 4H), 1.84-1.72 (m, 1H), 1.69-1.56 (m, 3H), 1.54-1.23 (m, 7H), 1.20-1.06 (m, 3H), 1.04-0.91 (m, 2H). |
| 188 | 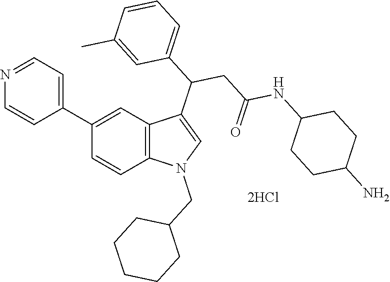 [SMILES: Cc1cccc(C(CC(=O)NC2CCC(N)CC2)c2cn(CC3CCCCC3)c3ccc(-c4ccncc4)cc23)c1 ; "2HCl"] | ¹H NMR (400 MHz, DMSO-d₆): δ 8.77(d, J = 6.1 Hz, 2H), 8.18 (bs, 2H), 8.03 (bs, 1H), 7.89-7.75 (m, 4H), 7.71 (d, J = 8.7 Hz, 1H), 7.62 (d, J = 8.7 Hz, 1H), 7.41(s, 1H), 7.25- 6.99 (m, 4H), 6.93 (d, J = 6.9 Hz, 1H), 4.79 (t, J = 8.7 Hz, 1H), 4.04 (d, J = 7.2 Hz, 2H), 3.04-2.84 (m, 2H), 2.72-2.63 (m, 2H), 2.21 (s, 3H), 1.96-1.41 (m, 10H), 1.35-1.20 (m, 2H), 1.17-0.97 (m, 7H). |
| 189 | 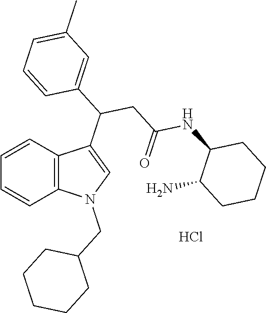 [SMILES: Cc1cccc(C(CC(=O)N[C@H]2CCCC[C@@H]2N)c2cn(CC3CCCCC3)c3ccccc23)c1 ; Cl] | ¹H NMR(400 MHz, Methanol-d₄): δ 7.39-7.29 (m, 2H), 7.19-7.06 (m, 5H), 7.03-6.87 (m, 2H), 3.99-3.93 (m, 2H), 3.64-3.55 (m, 2H), 3.07-3.00 (m, 1H), 2.96-2.86 (m, 1H), 2.85-2.75 (m, 1H), 2.27 (s, 3H), 2.06-1.96 (m, 1H), 1.90- 1.66 (m, 6H), 1.64-1.55 (m, 2H), 1.49-1.20 (m, 8H), 1.10- 0.99 (m, 2H); |
| 190 | 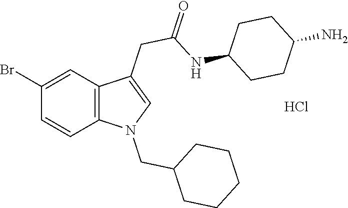 [SMILES: N[C@H]1CC[C@H](NC(=O)Cc2cn(CC3CCCCC3)c3ccc(Br)cc23)CC1 ; Cl] | ¹H NMR (400 MHz, DMSO-d₆): δ 7.96, (d, J = 7.6 Hz, 1H), 7.77-7.67 (m, 4H), 7.41 ((d, J = 9.0 Hz, 1H), 7.23-7.18 (m, 2H), 3.95 (d, J = 7.2 Hz, 2H), 3.51-3.41 (m, 3H), 3.03-2.93 (m, 1H), 1.96-1.88 (m, 2H), 1.85-1.77 (m, 2H), 1.76-1.68 (m, 1H), 1.67-1.58 (m, 3H), 1.50-1.40 (m, 2H), 1.37-1.20 (m, 4H), 1.16-1.08 (m, 3H), 1.02-0.88 (m, 2H); |
| 191 | 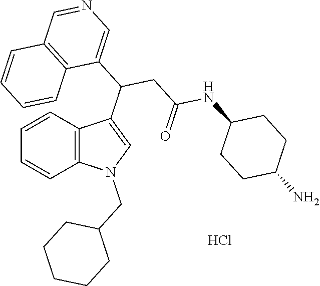 [SMILES: N[C@H]1CC[C@H](NC(=O)CC(c2cncc3ccccc23)c2cn(CC3CCCCC3)c3ccccc23)CC1 ; Cl] | ¹H NMR (400 MHz, DMSO-d₆): δ 8.89 (bs, 2H), 8.65 (bs, 1H), 8.27 (s, 1H), 7.97-7.91 (m, 4H), 7.72-7.67 (m, 1H), 7.56 (dt, J = 8.0, 1.2 Hz, 1H), 7.44-7.41 (m, 2H), 7.42 (s, 1H), 7.10-7.0 (m, 1H), 6.97-6.89 (m, 1H), 6.49 (s, 1H), 4.57 (t, J = 7.6 Hz, 1H), 3.99 (d, J = 7.2 Hz, 2H), 2.96- 2.76 (m, 5H), 2.08-1.94 (m, 4H), 1.82-1.80 (m, 1H), 1.65- 1.61 (m, 3H), 1.53-1.50 (m, 3H), 1.33-1.24 (m, 4H), 1.03 (bs, 3H), 1.0-0.98 (m, 2H); |
| 197 | 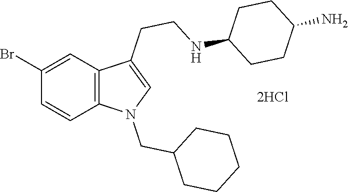 [SMILES: N[C@H]1CC[C@H](NCCc2cn(CC3CCCCC3)c3ccc(Br)cc23)CC1 ; "2HCl"] | ¹H NMR (400 MHz, DMSO-d₆): δ 8.88 (bs, 3H), 8.00 (bs, 2H), 7.79-7.78 (m, 1H), 7.45 (d, J = 8.6 Hz, 1H), 7.31 (s, 1H), 7.24 (dd, J = 8.76 1.86 Hz, 1H), 3.95 (d, J = 6.9 Hz, 2H), 3.18-3.10 (m, 2H), 3.05-3.01 (m, 3H), 2.99-2.90 (m, 1H), 2.06-1.97 (m, 2H), 1.95-1.79 (m, 2H) 1.80-1.70 (m, 1H), 1.68-1.58 (m, 3H), 1.51-1.33 (m, 6H), 1.14-1.08 (m, 3H), 1.03-0.93 (m, 2H); |
| 201 | 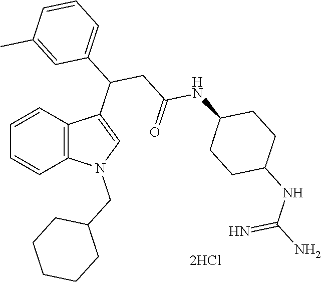 | ¹H NMR 400 MHz; DMSO-d₆): δ 7.76 (d, J = 7.6 Hz, 1H), 7.49 (d, J = 8.3 Hz, 1H), 7.37 (d, J = 8.3 Hz, 1H), 7.33-7.21 (m, 3H), 7.17 (s, 1H), 7.14-6.98 (m, 6H), 6.90-6.82 (m, 3H), 4.60 (t, J = 7.6 Hz, 1H), 3.95 (d, J = 7.0 Hz, 2H), 2.89-2.77 (m, 1H), 2.73-2.61 (m, 1H), 2.21 (s, 3H), 1.83-1.46 (m, 11H), 1.24-0.96 (m, 10H). |
| 202 | 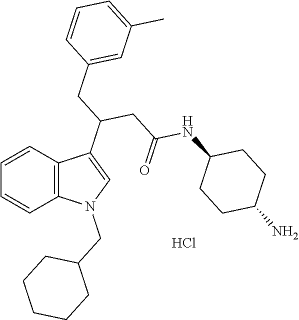 [SMILES: Cc1cccc(CC(CC(=O)N[C@H]2CC[C@H](N)CC2)c2cn(CC3CCCCC3)c3ccccc23)c1 ; Cl] | ¹H NMR (400 MHz, DMSO-d₆): δ 7.75 (bs, 3H), 7.70 (d, J = 7.6 Hz, 1H), 7.49 (d, J = 8.7 Hz, 1H), 7.42 (d, J = 1.7 Hz, 1H), 7.09-6.86 (m, 6H), 6.93 (d, J = 6.9 Hz, 1H), 3.88 (d, J = 6.7 Hz, 2H), 3.61 (t, J = 7.0 Hz, 1H), 3.40-3.29 (m, 1H), 2.96-2.58 (m, 3H) 2.41 (d, J = 7.7 Hz, 2H), 2.18 (s, 3H), 1.90-1.71 (m, 3H), 1.69-1.54 (m, 5H), 1.44-1.41 (m, 2H), 1.29-1.25 (m, 2H), 1.15-0.95 (m, 5H), 0.91-0.81 (m, 2H); |
| 203 | 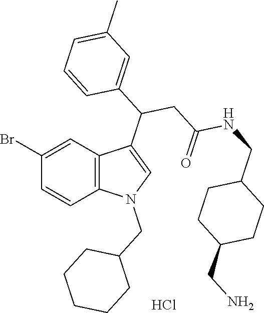 | ¹H NMR (400 MHz, DMSO-d₆): δ 7.76 (t, J = 5.9 Hz, 1H), 7.70 (bs, 3H), 7.44 (d, J = 1.9 Hz, 1H), 7.39 (d, J = 8.8 Hz, 1H), 7.32 (s, 1H), 7.19-7.02-(m, 4H), 6.95 (d, J = 7.2 Hz, 1H), 4.57 (t, J = 8.2 Hz, 1H), 4.00-3.90 (m, 2H), 2.96-2.56 (m, 6H), 2.22 (s, 3H), 1.80-1.55 (m, 6H), 1.53-1.33 (m, 5H), 1.20-1.03 (m, 4H), 1.02-0.89 (m, 2H), 0.85-0.62 (m, 4H). |
| 204 | 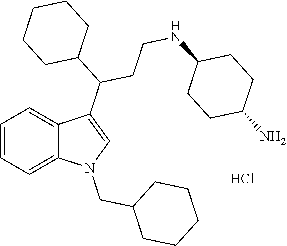 [SMILES: N[C@H]1CC[C@H](NCCC(c2cn(CC3CCCCC3)c3ccccc23)C2CCCCC2)CC1 ; Cl] | ¹H NMR (400 MHz, Methanol-d₄): δ 7.55 (d, J = 8.0 Hz, 1H), 7.34 (d, J = 8.0 Hz, 1H), 7.11 (dt, J = 7.8, 1.2 Hz, 1H), 7.03 (s, 1H), 6.99 (t, J = 7.2 Hz, 1H), 4.54 (s, 1H), 3.96 (d, J = 7.2 Hz, 2H), 3.10-3.04 (m, 1H), 2.89-2.84 (m, 1H), 2.75-2.68 (m, 3H), 2.17-2.11 (m, 2H), 1.99-1.84 (m, 5H), 1.82-1.78 (m, 1H), 1.70-1.69 (m, 2H), 1.65-1.62 (m, 2H), 1.57-1.54 (m, 3H), 1.45-1.43 (m, 1H), 1.39- 1.28 (m, 5H), 1.25-1.11 (m, 5H), 1.09-0.93 (m, 5H); |
| 205 | 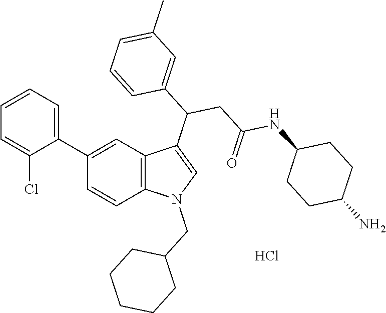 [SMILES: Cc1cccc(C(CC(=O)N[C@H]2CC[C@H](N)CC2)c2cn(CC3CCCCC3)c3ccc(-c4ccccc4Cl)cc23)c1 ; Cl] | ¹H NMR (400 MHz, DMSO-d₆): δ 7.76 (d, J = 7.5 Hz, 1H), 7.64 (bs, 3H), 7.53-7.48 (m, 1H), 7.46 (d, J = 8.8 Hz, 1H), 7.42-7.28 (m, 5H), 7.15-7.01 (m, 4H), 6.92 (d, J = 7.1 Hz, 1H), 4.65-4.58 (m, 1H), 3.99 (d, J = 7.3 Hz, 2H), 2.96-2.78 (m, 2H), 2.77-2.63 (m, 1H), 2.20 (s, 3H), 1.91-1.73 (m, 3H), 1.71-1.50 (m, 7H), 1.37-0.93 (m, 10H). |
| 206 | 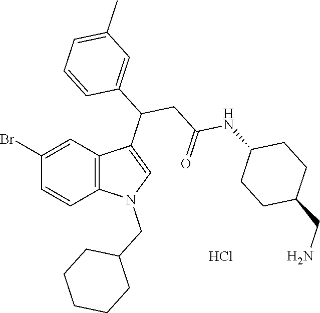 [SMILES: Cc1cccc(C(CC(=O)N[C@H]2CC[C@H](CN)CC2)c2cn(CC3CCCCC3)c3ccc(Br)cc23)c1 ; Cl] | ¹H NMR (400 MHz, DMSO-d₆): δ 7.77-7.60 (m, 4H), 7.37 (d, J = 8.7, 1H), 7.33-7.24 (m, 2H), 7.14-7.01 (m, 4H), 6.95- 6.83 (m, 2H), 4.60 (t, J = 7.6 Hz, 1H), 3.95 (d, J = 7.2 Hz, 2H), 3.41-3.35 (m, 1H), 2.89-2.79 (m, 1H), 2.69-2.60 (m, 3H), 2.21 (s, 3H), 1.82-1.60 (m, 8H), 1.57-1.37 (m, 3H), 1.19-1.08 (m, 3H), 1.07-0.91 (m, 6H). |
| 207 | 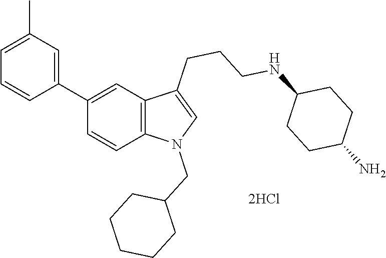 [SMILES: Cc1cccc(-c2ccc3c(c2)c(CCCN[C@H]2CC[C@H](N)CC2)cn3CC2CCCCC2)c1 ; "2HCl"] | ¹H NMR (400 MHz, DMSO-d₆): δ 7.72 (bs, 2H), 7.94 (bs, 3H), 7.77 (s, 1H), 7.53-7.37 (m, 4H), 7.32 (t, J = 7.6 Hz, 1H), 7.18 (s, 1H), 7.11 (d, J = 7.3 Hz, 1H), 3.98 (d, J = 7.2 Hz, 2H), 3.05-2.87 (m, 4H), 2.82 (t, J = 7.2 Hz, 2H), 2.38 (s, 3H), 2.13-1.92 (m, 6H), 1.83-1.72 (m, 1H), 1.73-1.57 (m, 3H), 1.55-1.45 (m, 2H), 1.43-1.28 (m, 4H), 1.19-1.08 (m, 3H), 1.04-0.92 (m, 2H). |
| 208 | 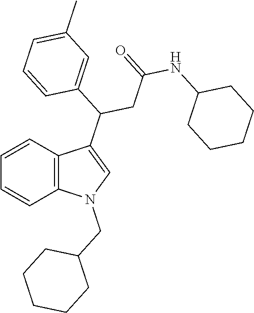 [SMILES: Cc1cccc(C(CC(=O)NC2CCCCC2)c2cn(CC3CCCCC3)c3ccccc23)c1] | ¹H NMR (400 MHz, CDCl₃): δ 7.40 (d, J = 7.6 Hz, 1H), 7.29 (s, 1H), 7.25-7.08 (m, 4H), 7.0-6.96 (m, 2H), 6.92 (s, 1H), 4.99 (d, J = 8.0 Hz, 1H), 4.64 (t, J = 8.0 Hz, 1H), 3.91-3.89 (m, 2H), 3.62-3.57 (m, 2H), 2.98 (q, J = 7.2 Hz, 1H), 2.77 (q, J = 7.6 Hz, 1H), 2.28 (s, 3H), 1.85-1.78 (m, 1H), 1.71-1.63 (m, 4H), 1.51-1.47 (m, 3H), 1.28- 1.13 (m, 6H), 1.07-0.97 (m, 4H), 0.81-0.73 (m, 2H). |
| 209 | 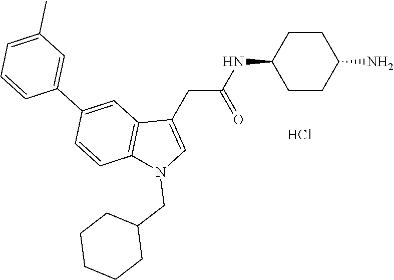 [SMILES: Cc1cccc(-c2ccc3c(c2)c(CC(=O)N[C@H]2CC[C@H](N)CC2)cn3CC2CCCCC2)c1 ; Cl] | %). ¹H NMR (400 MHz, Methanol-d₄): δ 7.90 (d, J = 7.3 Hz, 1H), 7.77 (s, 1H), 7.46-7.39 (m, 4H), 7.28 (t, J = 7.6 Hz, 1H), 7.13-7.08 (m, 2H), 3.98 (d, J = 7.3 Hz, 2H), 3.65 (s, 3H), 3.07-3.01 (m, 1H), 2.40 (s, 3H), 2.06-1.97 (m, 4H), 1.99-1.83 (m, 1H), 1.79-1.71 (m, 2H), 1.69-1.59 (m, 3H), 1.53-1.41 (m, 2H), 1.39-1.28 (m, 2H), 1.27-1.17 (m, 3H), 1.11-0.99 (m, 2H). |
| 210 | 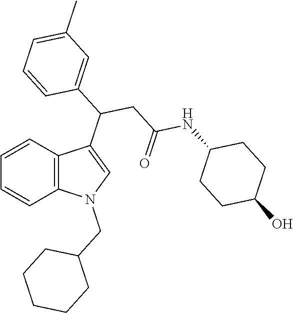 [SMILES: Cc1cccc(C(CC(=O)N[C@H]2CC[C@H](O)CC2)c2cn(CC3CCCCC3)c3ccccc23)c1] | ¹H NMR (400 MHz, DMSO-d₆): δ 7.63 (d, J = 7.75 Hz, 1H), 7.41-7.36 (m, 1H), 7.29-7.25 (m, 2H), 7.11-7.01 (m, 4H), 6.92-6.85 (m, 2H), 4.60 (t, J = 8.0 Hz, 1H), 4.46 (d, J = 4.4 Hz, 1H), 4.00-3.90 (m, 2H), 2.86-2.80 (m, 1H), 2.67- 2.60 (m, 1H), 2.21 (s, 3H), 1.77-1.70 (m, 3H), 1.68-1.55 (m, 6H), 1.52-1.43 (m, 2H), 1.15-1.06 (m, 6H), 1.04-0.92 (m, 3H). |
| 211 | 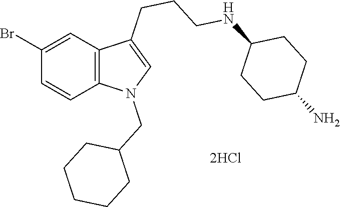 [SMILES: N[C@H]1CC[C@H](NCCCc2cn(CC3CCCCC3)c3ccc(Br)cc23)CC1] | ¹H NMR (400 MHz, DMSO-d₆): δ 8.84 (bs, 2H), 8.03 (bs, 3H), 7.73 (d, J = 1.8 Hz, 1H), 7.43 (d, J = 8.9 Hz, 1H), 7.31- 7.15 (m, 2H), 3.95 (d, J = 7.2 Hz, 2H), 3.06-2.85 (m, 4H), 2.74 (t, J = 7.3 Hz, 2H), 2.12-1.87 (m, 6H), 1.79-1.68 (m, 1H), 1.68-1.53 (m, 3H), 1.51-1.31 (m, 6H), 1.17-1.04 (m, 3H), 1.01-0.89 (m, 2H). |
| 215 | 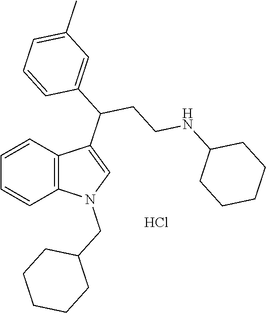 [SMILES: Cc1cccc(C(CCNC2CCCCC2)c2cn(CC3CCCCC3)c3ccccc23)c1 ; Cl] | ¹H NMR (400 MHz, Methanol-d₄): δ 7.36 (d, J = 8.0 Hz, 1H), 7.31 (d, J = 8.0 Hz, 1H), 7.19-7.06 (m, 5H), 6.99 (bs, 1H), 6.94-6.90 (m, 1H), 4.24 (t, J = 5.6 Hz, 1H), 3.97 (d, J = 7.2 Hz, 2H), 3.08-2.90 (m, 2H), 2.55-2.51 (m, 1H), 2.37-2.36 (m, 2H), 2.27 (s, 3H), 1.99-1.97 (m, 3H), 1.85- 1.80 (m, 4H), 1.72-1.66 (m, 2H), 1.59-1.56 (m, 2H), 1.37-1.30 (m, 3H), 1.29-1.19 (m, 5H), 1.04-0.99 (m, 2H). |
| 216 | 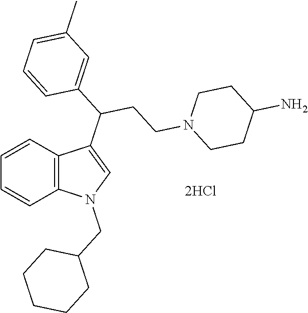 [SMILES: Cc1cccc(C(CCN2CCC(N)CC2)c2cn(CC3CCCCC3)c3ccccc23)c1 ; "2HCl"] | ¹H NMR (400 MHz, DMSO-d₆): δ 10.62 (bs, 1H), 8.23 (bs, 3H), 7.42-7.39 (m, 3H), 7.18-7.13 (m, 3H), 7.04 (dt, J = 8.0, 0.8 Hz, 1H), 6.96 (d, J = 6.4 Hz, 1H), 6.89 (t, J = 7.6 Hz, 1H), 4.15 (t, J = 7.6 Hz, 1H), 3.92-4.02 (m, 2H), 3.57- 3.46 (m, 2H), 3.21 (bs, 1H), 3.02-2.85 (m, 4H), 2.52- 2.50 (m, 1H), 2.49-2.46 (m, 2H), 2.04 (bs, 2H), 1.90 (bs, 2H), 1.81-1.76 (m, 1H), 1.65 (bs, 2H), 1.61 (bs, 1H), 1.54- 1.48 (m, 3H), 1.23 (s, 1H), 1.14-1.12 (m, 3H), 1.02- 0.97 (m, 2H). |
| 219 | 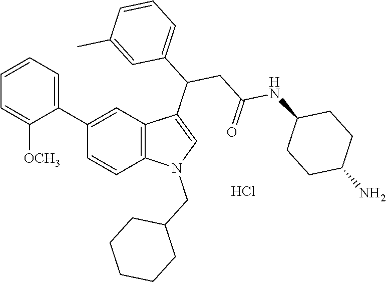 [SMILES: COc1ccccc1-c1ccc2c(c1)c(C(CC(=O)N[C@H]1CC[C@H](N)CC1)c1cccc(C)c1)cn2CC1CCCCC1 ; Cl] | ¹H NMR (400 MHz; DMSO-d₆): δ 7.80-7.70 (m, 4H), 7.40- 7.34 (m, 2H), 7.30-7.23 (m, 2H), 7.20-7.14 (m, 2H), 7.10 (t, J = 7.3 Hz, 1H), 7.03-7.01 (m, 3H), 6.98 (dt, J = 7.3, 1.0 Hz, 1H), 6.92 (d, J = 7.3 Hz, 1H), 4.61 (t, J = 7.7 Hz, 1H), 3.97 (d, J = 7.0 Hz, 2H), 3.67 (s, 3H), 3.42-3.33 (m, 1H), 2.98-2.76 (m, 2H), 2.73-2.62 (m, 1H), 2.21 (s, 3H), 1.92- 1.49 (m, 10H), 1.36-1.22 (m, 2H), 1.20-0.92 (m, 7H). |
| 221 | 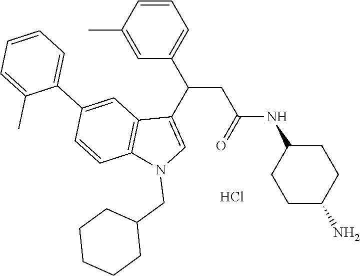 | ¹H NMR (400 MHz, DMSO-d₆): δ 7.76 (d, J = 7.52 Hz, 1H), 7.71 (bs, 3H), 7.42 (d, J = 8.3 Hz, 1H), 7.29 (s, 1H), 7.27-7.17 (m, 4H), 7.15-6.99 (m, 5H), 6.92 (d, J = 7.1 Hz, 1H), 4.61 (t, 1H, J = 8.1 Hz), 4.24-4.01 (m, 2H), 3.39-3.32 (m, 1H), 2.98-2.77 (m, 2H), 2.74-2.63 (m, 1H), 2.20 (s, 3H), 2.10 (s, 3H), 1.92-1.74 (m, 3H), 1.74-1.51 (m, 7H), 1.33-1.23 (m, 2H), 1.20-1.08 (m, 4H), 1.08-0.95 (m, 3H). |
| 222 | 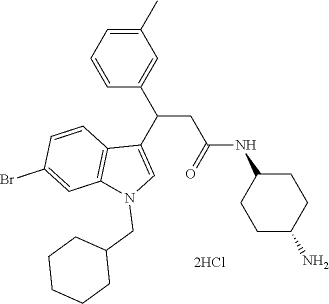 | ¹H NMR (400 MHz, DMSO-d₆): δ 8.77 (bs, 2H), 7.86 (bs, 3H), 7.70 (d, J = 1.2 Hz, 1H), 7.41-7.25 (m, 2H), 7.21-7.07 (m, 3H), 7.07-6.49 (m, 2H), 4.30-4.20 (m, 1H), 3.99 (d, J = 6.7 Hz, 2H), 3.03-2.82 (m, 3H), 2.80-2.71 (m, 1H), 2.23 (s, 3H), 2.07-1.90 (m, 4H), 1.83-1.72 (m, 1H), 1.70-1.56 (m, 3H), 1.54-1.21 (m, 7H), 1.18-0.92 (m, 7H).; |
| 223 | 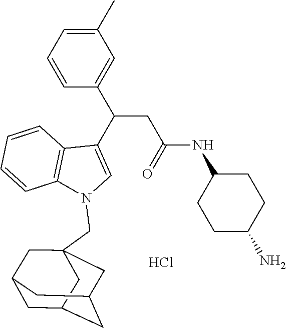 | ¹H NMR (400 MHz, DMSO-d₆): δ 7.78 (bs, 4H), 7.39 (d, J = 8.6 Hz, 1H), 7.26 (d, J = 7.7 Hz, 1H), 7.18 (s, 1H), 7.14- 7.06 (m, 1H), 7.06-6.99 (m, 3H), 6.92 (d, J = 7.3 Hz, 1H), 6.85 (t, J = 7.3 Hz, 1H), 4.61(t, J = 8.0 Hz, 1H), 3.80 (s, 2H), 3.02-2.78 (m, 2H), 2.70-2.56 (m, 1H), 2.20 (s, 3H), 1.96-1.81 (m, 5H), 1.72-1.59 (m, 5H), 1.59-1.46 (m, 9H), 1.38-1.21 (m, 3H), 1.19-1.00 (m, 2H). |
| 226 | 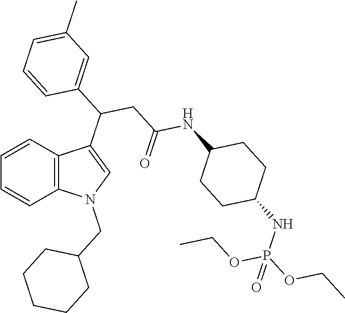 | ¹H NMR (400 MHz, DMSO-d₆): δ 7.70-7.65 (m, 1H), 7.37 (d, J = 8.3 Hz, 1H), 7.30-7.23 (m, 2H), 7.12-7.00 (m, 4H), 6.94-6.84 (m, 2H), 4.60 (t, J = 7.9 Hz, 1H), 4.14-405 (m, 2H), 4.04-3.92 (m, 3H), 3.89-3.81 (m, 2H), 2.87-2.80 (m, 1H), 2.68-2.62 (m, 1H), 2.21 (s, 3H), 1.82-1.71 (m, 3H), 1.69-1.57 (m, 5H), 1.55-1.47 (m, 2H), 1.28-1.23 (m, 4H), 1.22-1.17 (m, 5H), 1.16-1.09 (m, 5H), 1.05-0.94 (m, 3H). |
| 227 | 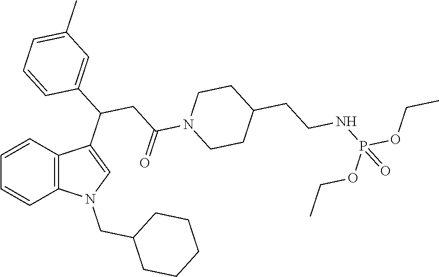 [SMILES: CCOP(=O)(NCCC1CCN(C(=O)CC(c2cccc(C)c2)c2cn(CC3CCCCC3)c3ccccc23)CC1)OCC] | ¹H NMR (400 MHz, DMSO-d₆): δ 7.39-7.23 (m, 3H), 7.14- 7.01 (m, 4H), 6.95-6.84 (m, 2H), 5.32-5.4 (m, 1H), 4.67- 4.59 (m, 1H), 4.29 (t, J = 14.2 Hz, 1H), 4.15-4.06 (m, 2H), 4.05-3.92 (m, 4H), 3.91-3.82 (m, 2H), 3.20-3.05 (m, 1H), 3.00-2.91 (m, 1H), 2.85-2.73 (m, 2H), 2.39-2.30 (m, 1H), 2.21 (s, 3H), 1.81-1.72 (m, 1H), 1.69-1.59 (m, 3H), 1.57- 1.43 (m, 5H), 1.29-1.18 (m, 9H), 1.16-1.10 (m, 3H), 1.02- 0.93 (m, 2H), 0.77-0.66 (m, 1H). |
| 228 | 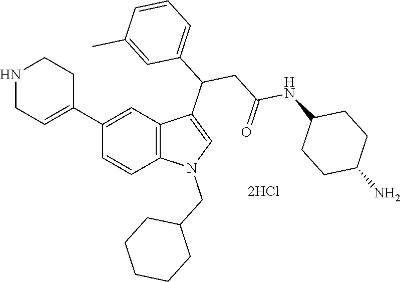 | ¹H NMR (400 MHz; DMSO-d₆): δ 8.99 (bs, 2H), 7.99-7.85 (m, 3H), 7.81 (d, J = 7.5 Hz, 1H), 7.38 (d, J = 8.8 Hz, 1H), 7.33 (d, J = 1.3 Hz, 1H), 7.27 (s, 1H), 7.21 (dd, J = 8.8, 1.6 Hz, 1H), 7.10 (d, J = 7.5 Hz, 1H), 7.08-7.03 (m, 2H), 6.93 (d, J = 6.2 Hz, 1H), 6.01-5.95 (m, 1H), 4.64 (t, J = 7.5 Hz, 1H), 3.95 (d, J = 7.2 Hz, 2H), 3.75-3.67 (m, 2H), 2.95-2.81 (m, 2H), 2.70-2.61 (m, 3H), 2.21 (s, 3H), 1.91-1.83 (m, 2H), 1.79-1.71 (m, 1H), 1.70-1.56 (m, 6H), 1.55-1.46 (m, 2H), 1.37-1.22 (m, 3H), 1.19-0.94 (m, 8H). |
| 229 | 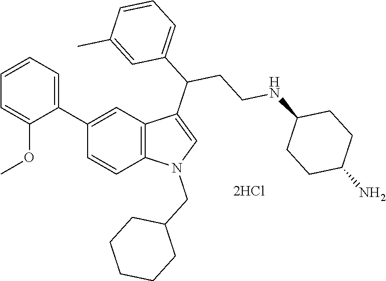 [SMILES: COc1ccccc1-c1ccc2c(c1)c(C(CCN[C@H]1CC[C@H](N)CC1)c1cccc(C)c1)cn2CC1CCCCC1 ; "2HCl"] | ¹H NMR (400 MHz, DMSO-d₆): δ 8.93 (bs, 2H), 7.95 (bs, 3H), 7.45 (d, J = 1.3 Hz, 1H), 7.41 (d, J = 8.5 Hz, 1H), 7.38 (s, 1H), 7.31-7.24 (m, 1H), 7.22-7.11 (m, 5H), 7.06 (d, J = 7.5 Hz, 1H), 7.02-6.95 (m, 2H), 4.27 (t, J = 7.5 Hz, 1H), 4.00 (d, J = 6.9 Hz, 2H), 3.68 (s, 3H), 3.02-2.88 (m, 3H), 2.85-2.72 (m, 1H), 2.36-2.28 (m, 1H), 2.24 (s, 3H), 2.06- 1.92(m, 4H), 1.87-1.76 (m, 1H), 1.71-1.26 (m, 10H), 1.20- 1.10 (m, 3H), 1.07-0.95 (m, 2H). |
| 230 | 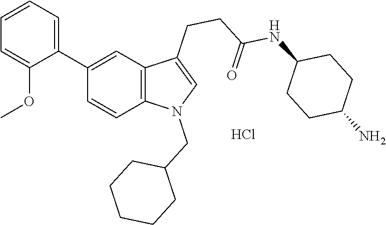 [SMILES: COc1ccccc1-c1ccc2c(c1)c(CCC(=O)N[C@H]1CC[C@H](N)CC1)cn2CC1CCCCC1 ; Cl] | ¹H NMR 400 MHz; DMSO-d₆): δ 7.80 (bs, 3H), 7.70 (d, J = 7.5 Hz, 1H), 7.53 (d, J = 1.3 Hz, 1H), 7.38 (d, J = 8.5 Hz, 1H), 7.33-7.26 (m, 2H), 7.21 (dd, J = 8.5, 1.6 Hz, 1H),7.12- 7.04 (m, 2H), 7.01 (dt, J = 7.5, 0.9 Hz, 1H), 3.93 (d, J = 6.9 Hz, 2H), 3.75 (s, 3H), 3.51-3.37 (m, 1H), 2.99-2.85 (m, 3H), 2.38 (t, J = 7.5 Hz, 2H), 1.94-1.83(m, 2H), 1.82-1.49 (m, 8H), 1.40-1.25 (m, 2H), 1.20-1.06 (m, 5H), 1.05-0.94 (m, 2H). |
| 231 | 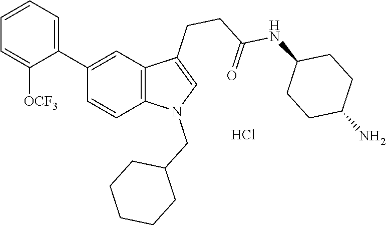 [SMILES: N[C@H]1CC[C@H](NC(=O)CCc2cn(CC3CCCCC3)c3ccc(-c4ccccc4OC(F)(F)F)cc23)CC1 ; Cl] | ¹H NMR (400 MHz, DMSO-d₆): δ 7.78 (bs, 3H), 7.72 (d, J = 7.6 Hz, 1H), 7.62-7.54 (m, 2H), 7.51-7.43 (m, 4H), 7.23- 7.19 (m, 1H), 7.12 (s, 1H), 3.96 (d, J = 6.8 Hz, 2H), 3.50- 3.38 (m, 1H), 2.91 (t, J = 7.5 Hz, 3H), 2.39 (t, J = 7.5 Hz, 2H), 1.91-1.82 (m, 2H), 1.82-1.71 (m, 3H), 1.70-1.48 (m, 5H), 1.39-1.23 (m, 2H), 1.20-1.06 (m, 5H), 1.06-0.93 (m, 2H). |
| 232 | 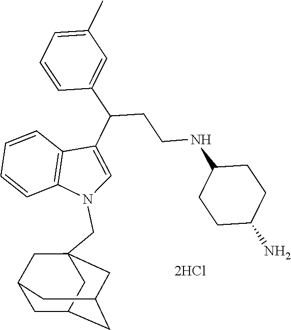 | ¹H NMR (400 MHz, DMSO-d₆): δ 9.02 (bs, 2H), 7.98 (bs, 3H), 7.42 (d, J = 8.0 Hz, 1H), 7.35 (d, J = 8.0 Hz, 1H), 7.30 (s, 1H), 7.19-7.09 (m, 3H), 7.08-7.01 (m, 1H), 6.97 (d, J = 7.1 Hz, 1H), 6.88 (t, J = 7.1 Hz, 1H), 4.27(t, J = 7.9 Hz, 1H), 3.85 (s, 2H), 3.02-2.87 (m, 3H), 2.86-2.74 (m, 1H), 2.35-2.28 (m, 1H), 2.23 (s, 3H), 2.04-1.89 (m, 7H), 1.71- 1.59 (m, 3H), 1.58-1.46 (m, 10H), 1.42-1.28 (m, 4H). |
| 234 | 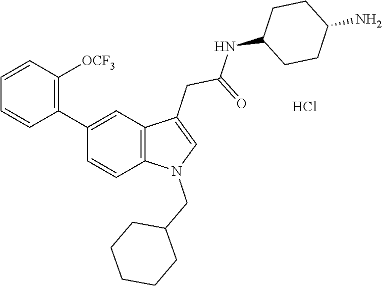 | ¹H NMR (400 MHz, DMSO-d₆): δ 7.91 (d, J = 7.6 Hz, 1H), 7.67-7.57 (m, 3H), 7.54-7.50 (m, 2H), 7.49-7.45 (m, 4H), 7.23-7.19 (m, 2H), 3.99 (d, J = 7.0 Hz, 2H), 3.47 (s, 3H), 2.98-2.89 (m, 1H), 1.93-1.86 (m, 2H), 1.84-1.74 (m, 3H), 1.70-1.64 (m, 2H), 1.63-1.54 (m, 3H), 1.38-1.27 (m, 2H), 1.25-1.13 (m, 5H), 1.06-0.96 (m, 2H); |
| 235 | 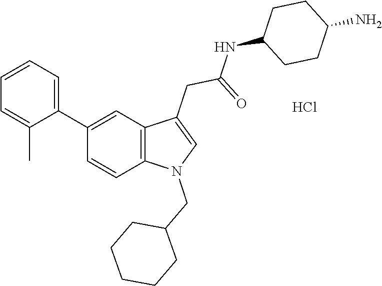 | ¹H NMR (400 MHz, DMSO-d₆): δ 7.91 (d, J = 7.6 Hz, 1H), 7.75 (bs, 3H), 7.47 (s, 1H), 7.43 (d, J = 8.4 Hz, 1H), 7.29- 7.25 (m, 1H), 7.25-7.19 (m, 3H), 7.18 (s, 1H), 7.06 (dd, J = 8.4 Hz, 1.6 Hz, 1H), 3.97 (d, J = 7.2 Hz, 2H), 3.50-3.40 (m, 3H), 2.97-2.91 (m, 1H), 2.25 (s, 3H), 1.96-1.94 (m, 2H), 1.90-1.88 (m, 3H), 1.79-1.55 (m, 5H), 1.38-1.29 (m, 2H), 1.37-1.11 (m, 5H), 1.04-0.98 (m, 2H); |
| 236 | 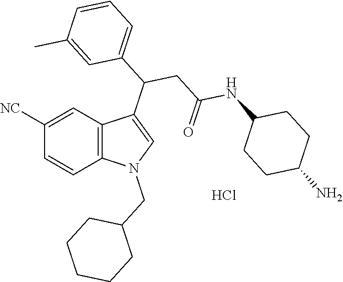 | ¹H NMR (400 MHz, DMSO-d₆): δ 7.81 (d, J = 1.2 Hz, 1H) 7.77 (s, 1H), 7.75 (bs, 3H), 7.60 (d, J = 8.4 Hz, 1H), 7.47 (s, 1H), 7.42 (dd, J = 8.4, 1.6 Hz, 1H), 7.15-7.07 (m, 3H), 6.94 (d, J = 7.2 Hz, 1H), 4.63 (t, J = 8.0 Hz, 1H), 4.02 (d, J = 6.8 Hz, 2H), 2.94-2.83 (m, 2H), 2.70-2.66 (m, 1H), 2.22 (s, 3H), 2.0-1.99 (m, 2H), 1.86-1.84 (m, 1H), 1.61 (bs, 4H), 1.44-1.14 (m, 2H), 1.33-1.22 (m, 4H), 1.17- 0..98 (m, 5H), 0.96-0.84 (m, 2H); |
| 237 | 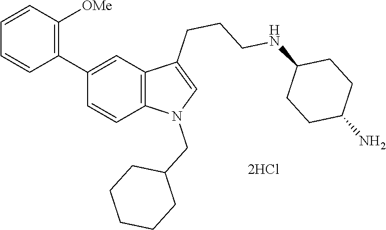 [SMILES: COc1ccccc1-c1ccc2c(c1)c(CCCN[C@H]1CC[C@H](N)CC1)cn2CC1CCCCC1] | ¹H NMR (400 MHz, DMSO-d₆): δ 7.80 (bs, 4H), 7.70 (d, J = 7.5 Hz, 1H), 7.53 (d, J = 1.3 Hz, 1H), 7.38 (d, J = 8.5 Hz, 1H), 7.33-7.26 (m, 2H), 7.21 (dd, J = 8.5, 1.6 Hz, 1H),7.12- 7.04 (m, 2H), 7.01 (dt, J = 7.5, 0.9 Hz, 1H), 3.93 (d, J = 6.9 Hz, 2H), 3.75 (s, 3H), 3.51-3.37 (m, 1H), 2.99-2.85 (m, 3H), 2.38 (t, J = 7.5 Hz, 2H), 1.94-1.83(m, 2H), 1.82-1.49 (m, 8H), 1.40-1.25 (m, 3H), 1.20-1.06 (m, 6H), 1.05-0.94 (m, 2H). |
| 238 | 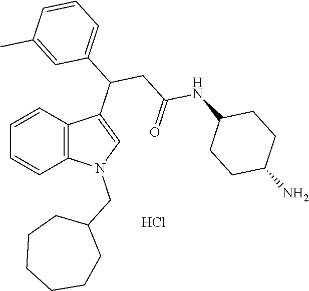 | ¹H NMR 400 MHz; DMSO-d₆): δ 7.92-7.69 (m, 4H), 7.36 (d, J = 8.3 Hz, 1H), 7.31-7.23 (m, 2H), 7.14-6.98 (m, 4H), 6.92 (d, J = 7.5 Hz, 1H), 6.87 (t, J = 7.3 Hz, 1H), 4.60 (t, J = 8.0 Hz, 1H), 3.93 (d, J = 7.5 Hz, 2H), 3.43-3.33 (m, 1H), 2.97-2.78 (m, 2H), 2.71-2.58 (m, 1H), 2.20 (s, 3H), 2.07- 1.93 (m, 1H), 1.92-1.82 (m, 2H), 1.71-1.40 (m, 10H), 1.38- 1.04 (m, 8H). |
| 240 | 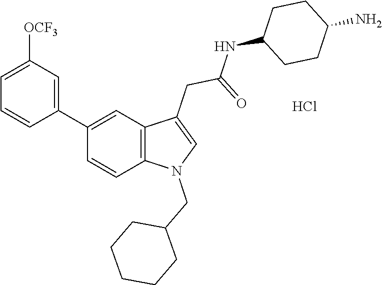 | ¹H NMR (400 MHz, DMSO-d₆): δ 8.00 (d, J = 8.1 Hz, 1H), 7.89 (d, J = 1.8 Hz, 1H), 7.82-7.61 (m, 4H), 7.60-7.50 (m, 3H), 7.48-7.43 (m, 1H), 7.31-7.27 (m, 1H), 7.21 (s, 1H), 3.99 (d, J = 7.1 Hz, 2H), 3.51 (s, 2H), 3.50-3.41 (m, 1H), 3.00-2.89 (m, 1H), 1.93-1.86 (m, 2H), 1.85-1.77 (m, 2H), 1.68-1.58 (m, 3H), 1.55-1.49 (m, 2H), 1.39-1.20 (m, 5H), 1.16-0.96 (m, 5H). |
| 241 | 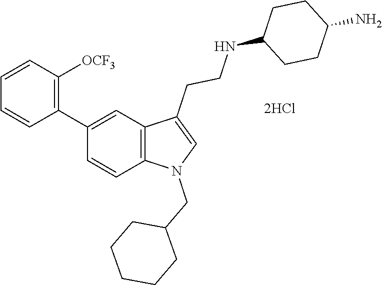 | ¹H NMR (400 MHz, DMSO-d₆): δ 8.87 (bs, 2H), 8.76 (bs, 1H), 7.97 (s, 2H), 7.67 (s, 1H), 7.60-7.54 (m, 2H), 7.51-7.47 (m, 3H), 7.34-7.23 (m, 2H), 3.99 (d, J = 7.0 Hz, 2H), 3.20- 3.13 (m, 2H), 3.11-3.04 (m, 3H), 2.99-2.92 (m, 1H), 2.17- 2.10 (m, 2H), 2.04-1.97 (m, 2H), 1.84-1.75 (m, 1H), 1.70- 1.65 (m, 2H), 1.63-1.54 (m, 2H), 1.48-1.31 (m, 5H), 1.19- 1.12 (m, 3H), 1.06-0.97 (m, 2H); |
| 242 | 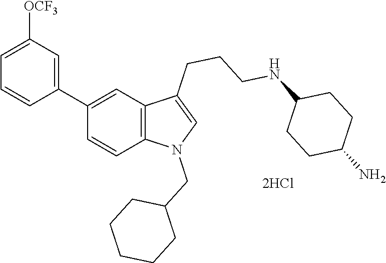 | ¹H NMR (400 MHz, DMSO-d₆): δ 8.78 (bs, 2H), 7.95 (bs, 3H), 7.87 (d, J = 1.4 Hz, 1H), 7.75 (d, J = 7.9 Hz, 1H), 7.63 (s, 1H), 7.60-7.51 (m, 2H), 7.49-7.42 (m, 1H), 7.31-7.26 (m, 1H), 7.22 (s, 1H), 3.99 (d, J = 6.9 Hz, 2H), 3.02-2.88 (m, 4H), 2.83 (t, J = 6.9 Hz, 2H), 2.15-1.93 (m, 6H), 1.86-1.74 (m, 1H), 1.72-1.57 (m, 3H), 1.55-1.47 (m, 2H), 1.45-1.28 (m, 4H), 1.21-1.07 (m, 3H), 1.05-0.93 (m, 2H). |
| 244 | 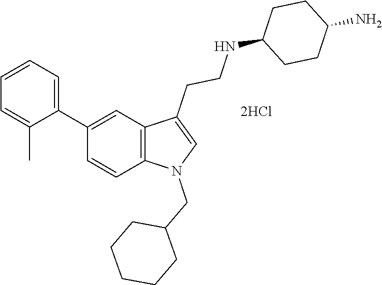 [SMILES: Cc1ccccc1-c1ccc2c(c1)c(CCN[C@H]1CC[C@H](N)CC1)cn2CC1CCCCC1 ; "2HCl"] | ¹H NMR (400 MHz, DMSO-d₆): δ 8.88 (bs, 2H), 7.96 (bs, 3H), 7.52 (s, 1H), 7.50 (d, J = 8.4 Hz, 1H), 7.30-7.28 (m, 2H), 7.25-7.22 (m, 3H), 7.10 (dd, J = 8.4, 1.6 Hz, 1H), 3.97 (d, J = 7.2 Hz, 2H), 3.14 (bs, 1H), 3.09-2.96 (m, 4H), 2.27 (s, 3H), 2.13 (bs, 1H), 2.11 (bs, 1H), 2.01 (bs, 1H), 1.98 (bs, 1H), 1.79-1.77 (m, 1H), 1.67-1.55 (m, 5H), 1.47-1.31 (m, 5H), 1.23-1.13 (m, 3H), 1.03-0.96 (m, 2H); |
| 245 | 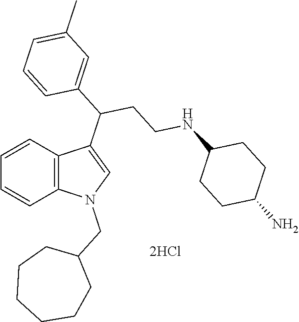 | ¹H NMR (400 MHz, DMSO-d₆): δ 9.19 (bs, 1H), 9.10 (bs, 1H), 8.06 (bs, 3H), 7.49-7.32 (m, 3H), 7.22-7.02 (m, 4H), 6.96 (d, J = 6.5 Hz, 1H), 6.90 (t, J = 7.8 Hz, 1H), 4.27 (t, J = 7.8 Hz, 1H), 3.96 (d, J = 7.5 Hz, 2H), 3.04-2.70 (m, 4H), 2.38-2.27 (m, 1H), 2.23 (s, 3H), 2.10-1.94 (m, 5H), 1.69- 1.25 (m, 15H), 1.24-1.13 (m, 2H). |
| 247 | 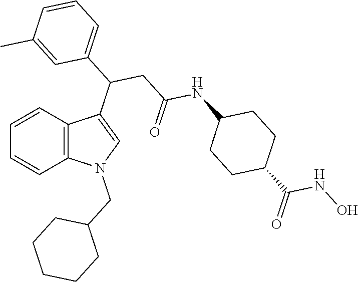 | ¹H NMR 400 MHz; DMSO-d₆): δ 10.28 (bs, 1H), 8.61 (bs, 1H), 7.67 (d, J = 7.8 Hz, 1H), 7.36 (d, J = 8.3 Hz, 1H), 7.28 (d, J = 8.0 Hz, 1H), 7.24 (s, 1H), 7.09 (t, J = 7.5 Hz, 1H), 7.06-6.99 (m, 3H), 6.91 (d, J = 7.3 Hz, 1H), 6.89-6.82 (m, 1H), 4.60 (t, J = 8.0 Hz, 1H), 3.95 (d, J = 7.0 Hz, 2H), 3.41- 3.31 (m, 1H), 2.89-2.76 (m, 1H), 2.70-2.59 (m, 1H), 2.20 (s, 3H), 1.93-1.70 (m, 2H), 1.69-1.47 (m, 9H), 1.41-1.29 (m, 2H), 1.17-0.93 (m, 7H). |
| 255 | 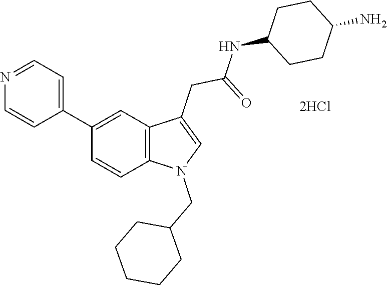 | ¹H NMR (400 MHz, DMSO-d₆): δ 8.80 (d, J = 8.8 Hz, 2H), 8.36-8.17 (m, 3H), 8.04 (d, J = 7.5 Hz, 1H,), 7.95-7.70 (m, 4H), 7.64 (d, J = 7.8 Hz, 1H), 7.28 (s, 1H), 4.03 (d, J = 7.0 Hz, 2H), 3.56 (s, 2H), 3.02-2.87 (m, 1H), 1.98-1.71 (m, 5H), 1.71-1.46 (m, 5H), 140-1.07 (m, 8H), 1.06-0.92 (m, 2H). |
| 259 | 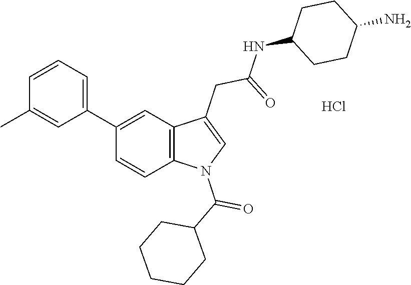 | ¹H NMR (400 MHz, DMSO-d₆): δ 8.30 (d, J = 8.1 Hz, 1H), 7.97-7.88 (m, 2H), 7.75 (bs, 3H), 7.33 (d, J = 7.0 Hz, 1H), 7.25 (t, J = 7.6 Hz, 1H),7.18-7.07 (m, 4H), 6.96 (d, J = 7.0 Hz, 1H), 4.66-4.58 (m, 1H), 3.47-3.36 (m, 1H), 3.06-2.86 (m, 2H), 2.73-2.61 (m, 1H), 2.23 (s, 3H), 1.98-1.67 (m, 9H), 1.61-1.45 (m, 4H), 1.37-1.08 (m, 6H). |
| 270 | 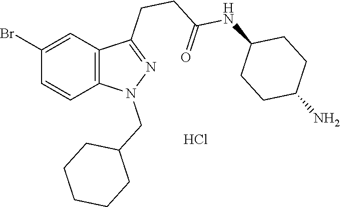 [SMILES: N[C@H]1CC[C@H](NC(=O)CCc2nn(CC3CCCCC3)c3ccc(Br)cc23)CC1 ; Cl] | ¹H NMR (400 MHz, DMSO-d₆): δ 7.87-7.68 (m, 4H), 7.67 (d, J = 1.9 Hz, 1H), 7.39 (d, J = 9.5 Hz, 1H), 7.21-7.17 (m, 1H), 7.12 (s, 1H), 3.92 (d, J = 7.2 Hz, 2H), 3.49-3.39 (m, 1H), 2.99-2.80 (m, 3H), 2.35-2.31(m, 2H), 1.95-1.83 (m, 2H), 1.81-1.71 (m, 2H), 1.68-1.57 (m, 3H), 1.51-1.20 (m, 5H), 1.19-1.06 (m, 5H), 0.97-0.84 (m, 2H); |
| 274 | 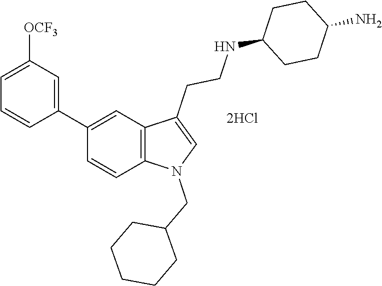 [SMILES: N[C@H]1CC[C@H](NCCc2cn(CC3CCCCC3)c3ccc(-c4cccc(OC(F)(F)F)c4)cc23)CC1 ; "2HCl"] | ¹H NMR (400 MHz, DMSO-d₆): δ 9.08 (bs, 2H), 8.00 (bs, 3H), 7.92 (s, 1H), 7.76 (d, J = 7.6 Hz, 1H), 7.64-7.53 (m, 3H), 7.51-7.46 (m, 1H), 7.33-7.29 (m, 2H), 3.99 (d, J = 6.9 Hz, 2H,), 3.25-3.10 (m, 4H), 3.06-2.90 (m, 2H), 2.15 (d, J = 10.2, 2H), 2.01 (d, J = 10.2 Hz, 2H), 1.86-1.72 (m, 1H), 1.71-1.29 (m, 9H), 1.20-1.08 (m, 3H), 1.06-0.94 (m, 2H). |
| 278 | 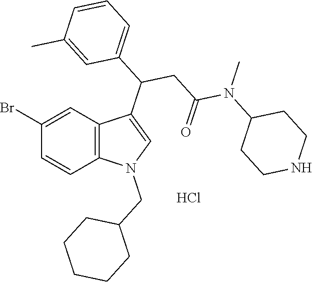 [SMILES: Cc1cccc(C(CC(=O)N(C)C2CCNCC2)c2cn(CC3CCCCC3)c3ccc(Br)cc23)c1 ; Cl] | ¹H NMR (400 MHz, DMSO-d₆): δ 8.55 (bs, 1H), 8.28 (bs, 1H), 7.50-7.25 (m, 3H), 7.21-7.08 (m, 4H), 6.99-6.92 (m, 1H), 4.63 (dd, J₁ = 6.6 Hz, J₂ = 6.6 Hz, 1H), 4.49-4.01 (m, 1H), 3.96 (d, J = 7.1 Hz, 2H), 3.29-3.05 (m, 3H), 3.04-2.84 (m, 3H), 2.78-2.54 (m, 3H), 2.22 (d, J = 3.7 Hz, 3H), 1.88- 1.37 (m, 10H), 1.21-1.05 (m, 3H), 1.03-0.87 (m, 2H). |
| 279 | 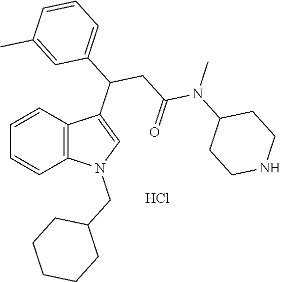 [SMILES: Cc1cccc(C(CC(=O)N(C)C2CCNCC2)c2cn(CC3CCCCC3)c3ccccc23)c1 ; Cl] | ¹H NMR (400 MHz, DMSO-d₆): δ 8.51 (bs, 2H), 7.19-7.40 (m, 3H), 7.01-7.17 (m, 4H), 6.82-6.96 (m, 2H), 4.62-4.69 (m, 1H), 4.10-3.99 (m, 1H), 3.95 (d, J = 6.8 Hz, 1H,), 3.17- 3.29 (m, 3H), 2.80-3.14 (m, 4H), 2.54-2.79 (m, 3H), 2.21 (d, J = 3.6 Hz, 3H,), 1.27-1.90 (m, 10H), 0.88-1.21 (m, 5H). |
| 280 | 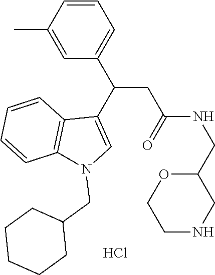 [SMILES: Cc1cccc(C(CC(=O)NCC2CNCCO2)c2cn(CC3CCCCC3)c3ccccc23)c1 ; Cl] | ¹H NMR (400 MHz, DMSO-d₆): δ 8.82 (bs, 2H), 8.08 (t, J = 6.0 Hz, 1H), 7.36 (d, J = 8.4 Hz, 1H), 7.29 (d, J = 8.0 Hz, 1H), 7.25 (s, 1H), 7.13-7.02 (m, 4H), 6.94-6.86 (m, 2H), 4.59 (t, J = 8.0 Hz, 1H), 3.97-3.87 (m, 3H), 3.60-3.55 (m, 2H), 3.12-2.95 (m, 2H), 2.93-2.76 (m, 4H), 2.21 (s, 3H), 1.90-1.48 (m, 6H), 1.26-1.23 (m, 2H), 1.13-1.11 (m, 3H), 1.01-0.95 (m, 2H); |
| 281 | 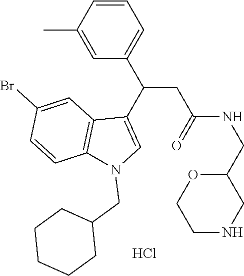 [SMILES: Cc1cccc(C(CC(=O)NCC2CNCCO2)c2cn(CC3CCCCC3)c3ccc(Br)cc23)c1 ; Cl] | ¹H NMR (400 MHz, DMSO-d₆): δ 8.79 (bs, 2H), 8.08 (t, J = 6.0 Hz, 1H), 7.43 (d, J = 2.0 Hz, 1H), 7.39 (d, J = 8.8 Hz, 1H), 7.34 (s, 1H), 7.17-7.12 (m, 2H), 7.05-7.09 (m, 2H), 6.94 (d, J = 7.2 Hz, 1H), 4.57 (t, J = 8.0 Hz, 1H), 3.98- 3.88 (m, 2H), 3.62-3.53 (m, 2H), 3.12-3.06 (m, 1H), 3.02- 2.92 (m, 2H), 2.90-2.76 (m, 3H), 2.22 (s, 3H), 1.81- 1.60 (m, 4H), 1.48-1.45 (m, 2H), 1.27-1.23 (m, 4H), 1.18- 1.10 (m, 2H), 1.05-0.94 (m, 2H); |
| 282 | 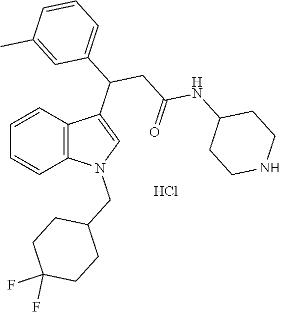 [SMILES: Cc1cccc(C(CC(=O)NC2CCNCC2)c2cn(CC3CCC(F)(F)CC3)c3ccccc23)c1 ; Cl] | ¹H NMR (400 MHz, DMSO-d₆): δ 8.49 (bs, 2H), 7.97 (d, J = 7.6 Hz, 1H), 7.41 (d, J = 8.0 Hz, 1H), 7.29 (d, J = 8.0 Hz, 1H), 7.27 (s, 1H), 7.12-7.04 (m, 4H), 6.94-6.87 (m, 2H), 4.59 (t, J = 8.0 Hz, 1H), 4.04 (d, J = 7.2 Hz, 2H), 3.73- 3.68 (m, 1H), 3.17-3.08 (m, 2H), 2.91-2.73 (m, 2H), 2.67- 2.66 (m, 1H), 2.21 (s, 3H), 1.95-1.81 (m, 2H), 1.78- 1.61 (m, 4H), 1.58-1.50 (m, 2H), 1.48-1.45 (m, 1H), 1.42- 1.41 (m, 1H), 1.38-1.22 (m, 4H); |
| 284 | 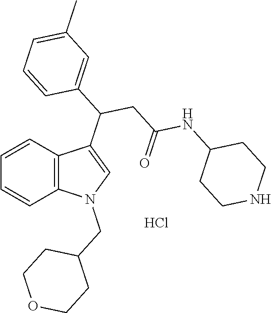 [SMILES: Cc1cccc(C(CC(=O)NC2CCNCC2)c2cn(CC3CCOCC3)c3ccccc23)c1 ; Cl] | ¹H NMR 400 MHz; DMSO-d₆): δ 8.62 (bs, 2H), 8.01 (d, J = 7.5 Hz, 1H), 7.42 (d, J = 8.4 Hz, 1H), 7.33-7.23 (m, 2H), 7.17-6.99 (m, 4H), 6.96-6.82 (m, 2H), 4.61 (t, J = 7.8 Hz, 1H), 4.02 (d, J = 7.1 Hz, 2H), 3.87-3.77 (m, 2H), 3.76-3.66 (m, 1H), 3.26-3.06 (m, 4H), 2.96-2.81 (m, 3H), 2.79-2.68 (m, 1H), 2.21 (s, 3H), 2.09-1.93 (m, 1H), 1.77-1.61 (m, 2H), 1.53-1.22 (m, 6H). |
| 285 | 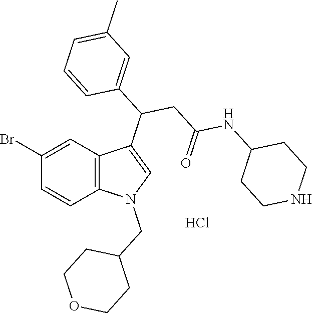 [SMILES: Cc1cccc(C(CC(=O)NC2CCNCC2)c2cn(CC3CCOCC3)c3ccc(Br)cc23)c1 ; Cl] | ¹H NMR 400 MHz; DMSO-d₆): δ 8.49 (bs, 2H), 7.99 (d, J = 7.6 Hz, 1H), 7.51-7.39 (m, 2H), 7.35 (s, 1H), 7.21-7.00 (m, 4H), 6.95 (d, J = 7.6 Hz, 1H), 4.59 (t, J = 8.2 Hz, 1H), 4.03 (d, J = 7.3 Hz, 2H), 3.86-3.77 (m, 2H), 3.76-3.62 (m, 1H), 3.25-3.07 (m, 4H), 2.96-2.78 (m, 3H), 2.77-2.67 (m, 1H), 2.22 (s, 3H), 2.06-1.92 (m, 1H), 1.75-1.60 (m, 2H), 1.51-1.22 (m, 6H). |
| 286 | 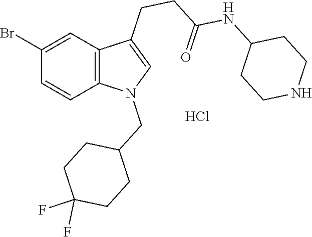 [SMILES: O=C(CCc1cn(CC2CCC(F)(F)CC2)c2ccc(Br)cc12)NC1CCNCC1 ; Cl] | ¹H NMR (400 MHz, DMSO-d₆): δ 8.41 (bs, 2H), 7.91 (d, J = 8.0 Hz, 1H), 7.70 (d, J = 4.0 Hz, 1H), 7.46 (d, J = 8.0 Hz, 1H), 7.20 (dd, J = 4.0, 8.0 Hz, 1H), 7.16 (s, 1H), 4.02 (d, J = 8.0 Hz, 2H), 3.78-3.76 (m, 1H), 3.41-3.38 (m, 2H), 3.17- 3.14 (m, 3H), 2.93-2.83 (m, 3H), 2.38 (t, J = 8.0 Hz, 2H), 2.02-1.92 (m, 2H), 1.85-1.18 (m, 9H); |
| 287 | 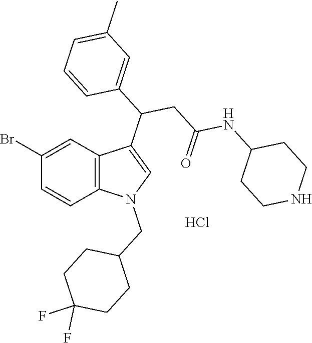 [SMILES: Cc1cccc(C(CC(=O)NC2CCNCC2)c2cn(CC3CCC(F)(F)CC3)c3ccc(Br)cc23)c1 ; Cl] | ¹H NMR (400 MHz, DMSO-d₆): δ 8.48 (bs, 2H), 7.98 (d, J = 8.0 Hz, 1H), 7.46 (s, 1H), 7.44 (d, J = 4.0 Hz, 1H), 7.36 (s, 1H), 7.18 (dd, J = 4.0, 8.0 Hz, 1H), 7.12 (d, J = 8.0 Hz, 1H), 7.06 (s, 1H), 7.05 (d, J = 8.0 Hz, 1H), 6.95 (d, J = 8.0 Hz, 1H), 4.59 (t, J = 8.0 Hz, 1H), 4.06 (d, J = 8.0 Hz, 2H), 3.72-3.69 (m, 1H), 3.16-3.09 (m, 2H), 2.88-2.74 (m, 4H), 2.22 (s, 3H), 1.98-1.90 (m, 3H), 1.77-1.67 (m, 4H), 1.55- 1.38 (m, 4H), 1.24-1.17 (m, 2H); |
| 288 | 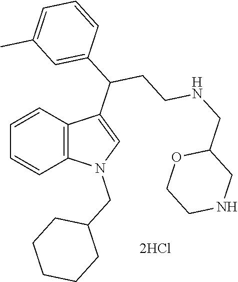 [SMILES: Cc1cccc(C(CCNCC2CNCCO2)c2cn(CC3CCCCC3)c3ccccc23)c1 ; "2HCl"] | ¹H NMR (400 MHz, Methanol-d₄): δ 7.37 (d, J = 8.0, 0.8 Hz, 1H), 7.31 (d, J = 8.0 Hz, 1H), 7.17-7.08 (m, 5H), 6.99 (d, J = 6.8 Hz, 1H), 6.90 (t, J = 7.6 Hz, 1H), 4.23 (t, J = 8.0 Hz, 1H), 4.16-4.09 (m, 2H), 3.96 (d, J = 7.2 Hz, 2H), 3.80 (t, J = 12.0 Hz, 1H), 3.70-3.61 (m, 1H), 3.40-3.35 (m, 1H), 3.20-3.10 (m, 4H), 3.07-2.97 (m, 2H), 2.60-2.56 (m, 1H), 2.44-2.41 (m, 1H), 2.27 (s, 3H), 1.87-1.82 (m, 1H), 1.71-1.58 (m, 5H), 1.26-1.20 (m, 3H), 1.04-0.98 (m, 2H); |
| 289 | 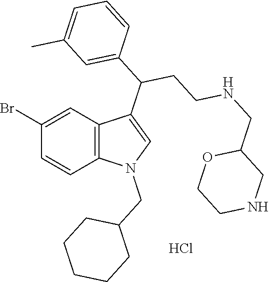 [SMILES: Cc1cccc(C(CCNCC2CNCCO2)c2cn(CC3CCCCC3)c3ccc(Br)cc23)c1 ; Cl] | ¹H NMR (400 MHz, DMSO-d₆): δ 9.27 (bs, 2H), 8.92 (bs, 2H), 7.53 (d, J = 1.6 Hz, 1H), 7.41 (d, J = 8.8 Hz, 1H), 7.36 (s, 1H), 7.17 (dd, J = 8.4, 2.0 Hz, 2H), 7.13-7.10 (m, 2H), 6.99 (d, J = 7.2 Hz, 1H), 4.22 (t, J = 8.0 Hz, 1H), 4.08 (t, J = 8.8 Hz, 1H), 4.01-3.94 (m, 3H), 3.77-3.71 (m, 1H), 3.22-3.11 (m, 3H), 3.0-2.89 (m, 3H), 2.85-2.77 (m, 2H), 2.45-2.41 (m, 1H), 2.24-2.20 (m, 1H), 2.21 (s, 3H), 1.73-1.71 (m, 1H), 1.72-1.60 (m, 3H), 1.46 (bs, 2H), 1.16-1.12 (m, 3H), 01.0-0.95 (m, 2H); |
| 290 | 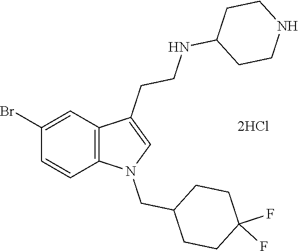 [SMILES: FC1(F)CCC(Cn2cc(CCNC3CCNCC3)c3cc(Br)ccc32)CC1] | ¹H NMR (400 MHz, DMSO-d₆): δ 9.20-8.56 (bs, 4H), 7.80 (d, J = 1.9 Hz, 1H), 7.51 (d, J = 8.9 Hz, 1H), 7.33 (s, 1H), 7.26 (dd, J = 6.8, 1.9 Hz, 1H), 4.06 (d, J = 6.8 Hz, 2H), 3.32-3.41 (m, 3H), 3.79-320 (m, 6H), 2.25-2.11-(m, 2H), 2.06-1.18 (m, 3H), 1.84-1.49 (m, 6H), 1.32-1.19 (m, 2H). |
| 291 | 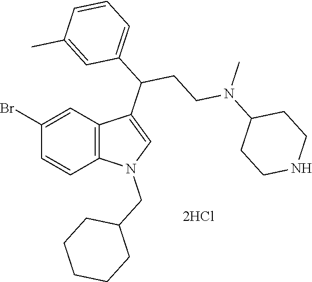 [SMILES: Cc1cccc(C(CCN(C)C2CCNCC2)c2cn(CC3CCCCC3)c3ccc(Br)cc23)c1] | ¹H NMR (400 MHz, DMSO-d₆): δ 10.98 (bs, 1H), 8.91 (bs, 1H), 8.66 (bs, 1H), 7.60-7.51 (m, 2H), 7.43 (d, J = 8.6 Hz, 1H), 7.23-7.10 (m, 4H), 7.02-6.96 (d, J = 5.6 Hz, 1H), 4.28- 4.18 (m, 1H), 3.98 (d, J = 7.2 Hz, 2H), 3.59-3.45 (m, 1H), 3.41-3.28 (m, 2H), 3.17-2.81 (m, 4H), 2.69 (m, 3H), 2.47- 2.29 (m, 2H), 2.25 (m, 3H), 2.19-2.01 (m, 2H), 1.94-1.71 (m, 3H), 1.71-1.55 (m, 3H), 1.54-1.42 (m, 2H), 1.21-0.92 (m, 5H). |
| 292 | 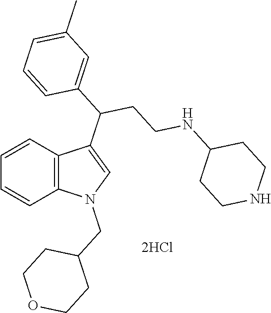 [SMILES: Cc1cccc(C(CCNC2CCNCC2)c2cn(CC3CCOCC3)c3ccccc23)c1] | ¹H NMR (400 MHz, DMSO-d₆): δ 9.08 (bs, 2H), 8.03 (bs, 3H), 7.43-7.34 (m, 3H), 7.20-7.10 (m, 3H), 7.09-7.03 (m, 1H), 6.99-6.94 (m, 1H), 6.90 (t, J = 7.2 Hz, 1H), 4.26 (t, J = 7.6 Hz, 1H), 3.98 (d, J = 6.9 Hz, 2H), 3.01-2.86 (m, 3H), 2.84-2.72 (m, 1H), 2.48-2.40 (m, 1H), 2.37-2.27 (m, 1H), 2.23 (s, 3H), 2.09-1.93 (m, 4H), 1.86-1.72 (m, 1H), 1.71- 1.55 (m, 3H), 1.54-1.29 (m, 6H), 1.21-1.06 (m, 3H), 1.05- 0.89 (m, 2H). |
| 293 | 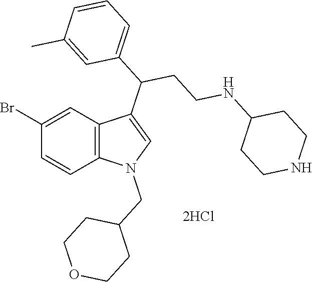 [SMILES: Cc1cccc(C(CCNC2CCNCC2)c2cn(CC3CCOCC3)c3ccc(Br)cc23)c1] | ¹H NMR (400 MHz, DMSO-d₆): δ 9.92-8.63 (m, 4H), 7.72- 7.38 (m, 3H), 7.35-6.83 (m, 5H), 4.44-4.21 (m, 1H), 4.19 (m, 2H), 3.93-3.71 (m, 2H), 3.26-3.12 (m, 6H), 3.03-2.70 (m, 4H), 2.40-1.62 (m, 9H), 1.50-1.03 (m, 4H). |
| 295 | 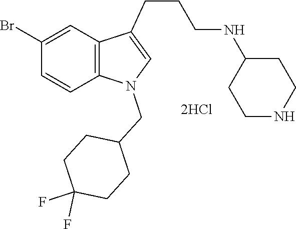 [SMILES: FC1(F)CCC(Cn2cc(CCCNC3CCNCC3)c3cc(Br)ccc32)CC1 ; "2HCl"] | ¹H NMR (400 MHz, DMSO-d₆): δ 8.91 (bs, 4H), 7.74 (s, 1H), 7.48 (d, J = 8.0 Hz, 1H), 7.25 (s, 1H), 7.23 (dd, J = 4.0, 8.0 Hz, 1H), 4.05 (d, J = 8.0 Hz, 2H), 3.01-2.85 (m, 5H), 2.74 (t, J = 8.0 Hz, 2H), 2.15-2.12 (m, 3H), 1.96-1.90 (m, 5H), 1.78-1.53 (m, 7H), 1.26-1.23 (m, 2H). |
| 296 | 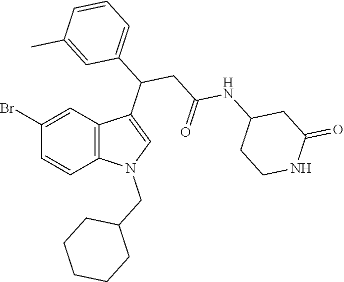 [SMILES: Cc1cccc(C(CC(=O)NC2CCNC(=O)C2)c2cn(CC3CCCCC3)c3ccc(Br)cc23)c1] | ¹H NMR (400 MHz, CDCl₃): δ 7.52 (dd, J = 4.0, 8.0 Hz, 1H), 7.23-7.21 (m, 1H), 7.18-7.13 (m, 2H), 7.08 (s, 1H), 7.06-7.02 (m, 2H), 6.90 (d, J = 8.0 Hz, 1H), 5.56 (s, 1H), 5.19 (t, J = 8.0 Hz, 1H), 4.64-4.60 (m, 1H), 4.13-4.11 (m, 1H), 3.90-3.82 (m, 2H), 3.18-3.14 (m, 1H), 3.02-2.76 (m, 3H), 2.48-2.44 (m, 1H), 2.30 (s, 3H), 1.86-1.67 (m, 6H), 1.47-1.44 (m, 1H), 1.19-1.14 (m, 3H), 1.00-0.94 (m, 2H). |
| 297 | 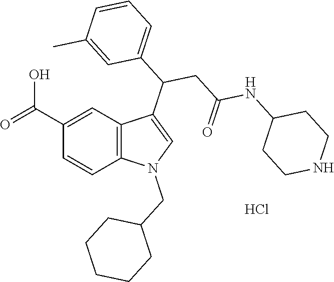 [SMILES: Cc1cccc(C(CC(=O)NC2CCNCC2)c2cn(CC3CCCCC3)c3ccc(C(=O)O)cc23)c1 ; Cl] | ¹H NMR (400 MHz, DMSO-d₆): δ 12.32 (bs, 1H), 8.39 (bs, 2H), 8.05-7.95 (m, 2H), 7.73-7.65 (m, 1H), 7.53-7.45 (m, 1H), 7.42-7.35 (m, 1H), 7.13 (t, J = 7.2 Hz, 1H), 7.09-7.02 (m, 2H), 6.95 (d, J = 7.6 Hz, 1H), 4.72-4.63 (m, 1H), 4.07- 3.93 (m, 2H), 3.77-3.65 (m, 1H), 3.23-3.05 (m, 2H), 2.97- 2.70 (m, 4H), 2.21 (s, 3H), 1.80-1.70 (m, 2H), 1.69-1.59 (m, 3H), 1.55-1.44 (m, 3H), 1.42-1.33 (m, 1H), 1.25-1.12 (m, 4H), 1.04-0.93 (m, 2H). |
| 298 | 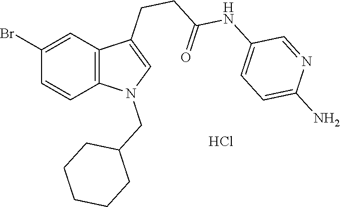 [SMILES: Nc1ccc(NC(=O)CCc2cn(CC3CCCCC3)c3ccc(Br)cc23)cn1 ; Cl] | ¹H NMR (400 MHz, DMSO-d₆): δ 9.52 (s, 1H), 8.01 (d, J = 4.0 Hz, 1H), 7.73 (d, J = 4.0 Hz, 1H), 7.51 (dd, J = 4.0, 8.0 Hz, 1H), 7.39 (d, J = 8.0 Hz, 1H), 7.19 (dd, J = 4.0, 8.0 Hz, 1H), 7.16 (s, 1H), 6.37 (d, J = 8.0 Hz, 1H), 3.92 (d, J = 8.0 Hz, 2H), 2.96 (t, J = 8.0 Hz, 2H), 2.55 (t, J = 8.0 Hz, 2H), 1.69-1.68 (m, 1H), 1.58-1.56 (m, 3H), 1.45-1.42 (m, 2H), 1.06-1.04 (m, 3H), 0.94-0.89 (m, 2H). |
| 299 | 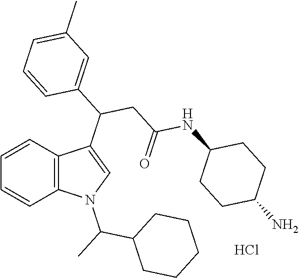 [SMILES: Cc1cccc(C(CC(=O)N[C@H]2CC[C@H](N)CC2)c2cn(C(C)C3CCCCC3)c3ccccc23)c1 ; Cl] | ¹H NMR (400 MHz, DMSO-d₆): δ 7.92-7.73 (m, 4H), 7.46- 7.36 (m, 2H), 7.28-7.22 (m, 1H), 7.13-7.08 (m, 1H), 7.06- 6.98 (m, 3H), 6.91 (d, J = 6.9 Hz, 1H), 6.85 (t, J = 7.3 Hz, 1H), 4.64-4.60 (m, 1H), 4.33-4.23 (m, 1H), 2.92-2.83 (m, 2H), 2.68-2.57 (m, 3H), 2.20 (d, J = 10.7 Hz, 3H), 1.91-1.80 (m, 3H), 1.76-1.66 (m, 3H), 1.62-1.55 (m, 2H), 1.47 (t, J = 6.3 Hz, 3H), 1.34-1.21 (m, 3H), 1.16-1.02 (m, 6H), 0.87- 0.76 (m, 1H). |
| 300 | 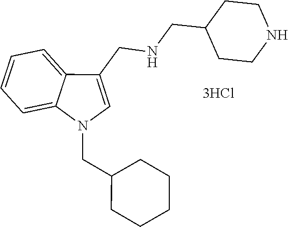 [SMILES: c1ccc2c(c1)c(CNCC1CCNCC1)cn2CC1CCCCC1 ; "3HCl"] | ¹H NMR (400 MHz, DMSO-d₆): δ 0.9-1.05 (m, 2H), 1.05- 1.21 (m, 3H), 1.3-1.4 (m, 2H), 1.45-1.55 (m, 2H), 1.55-1.6 (m, 2H), 1.95-2.05 (m, 1H), 2.85-2.95 (m, 4H), 3.25 (d, J = 12.56 Hz, 2H), 4.02 (d, J = 7.09 Hz, 2H), 4.28 (s, 2H), 7.10 (t, J = 7.22 Hz, 1H), 7.19 (t, J = 7.65 Hz, 1H), 7.51 (d, J = 7.65 Hz, 1H), 7.6 (s, 1H), 7.78 (d, J = 7.89 Hz, 1H), 8.85(brs, 1H), 9.0(brs, 1H), 9.15 (brs, 2H). |
| 301 | 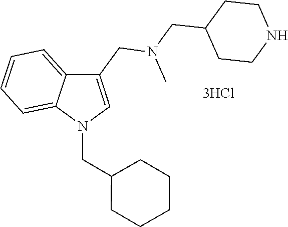 [SMILES: CN(Cc1cn(CC2CCCCC2)c2ccccc12)CC1CCNCC1 ; "3HCl"] | ¹H NMR (400 MHz, DMSO-d₆): δ 0.98-1.12 (m, 2H), 1.12- 1.23(m, 3H), 1.26-1.41(m, 3H) 1.50 (m, 2H) 1.60-1.75 (m, 3H), 1.75-1.90 (m, 2H), 2.02-2.15 (m, 2H), 2.68 (s, 3H), 2.82-2.88 (m, 3H), 2.88-3.22 (m, 1H), 3.23 (d, J = 11.29 Hz, 2H), 4.01-4.1(m, 2H), 4.41-4.49 (m, 2H), 7.11-7.22 (m, 2H), 7.55 (d, J = 8.15 Hz, 1H), 7.69(s, 1H), 7.80(d, J = 7.78 Hz, 1H), 8.82 (brs, 1H), 8.98 (brs, 1H), 10.27 (brs, 1H). |
| 302 | 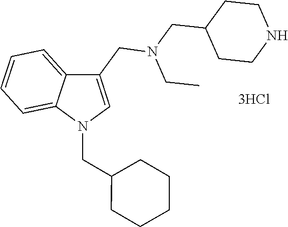 [SMILES: CCN(Cc1cn(CC2CCCCC2)c2ccccc12)CC1CCNCC1 ; "3HCl"] | ¹H NMR (400 MHz, DMSO-d₆): δ 0.98-1.03 (m, 2H), 1.12- 1.18(m, 5H), 1.20-1.47(m, 6H), 1.50 (d, , J = 10.63 Hz, 2H), 1.60-1.65 (m, 3H), 1.80-1.81 (m, 1H), 1.86-1.96 (m, 1H), 1.99-2.14 (m, 1H), 2.1-2.2 (m, 1H), 2.82-2.99 (m, 4H), 3.11-3.14 (m, 2H), 3.14-3.18(m, 2H), 3.18-3.29 (m, 2H), 4.05-4.06 (m, 2H), 4.50 (s 2H), 7.17(t, J = 7.64 Hz, 1H), 7.2(t, J = 7.78 Hz, 1H), 7.55(d, J = 8.20 Hz, 1H), 7.66 (s, 1H), 7.75(d, J = 7.90 Hz, 1H), 8.81 (brs, 1H), 9.02 (brs, 1H), 10.16 (brs, 1H). |
| 303 | 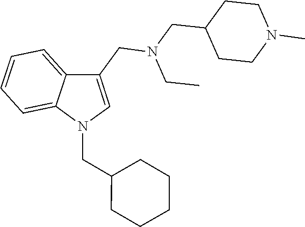 [SMILES: CCN(Cc1cn(CC2CCCCC2)c2ccccc12)CC1CCN(C)CC1] | ¹H NMR (400 MHz, DMSO-d₆): δ 0.85-1.0 (m, 7H), 1.11- 1.20(m, 4H) 1.23(s, 2H) 1.46-1.49 (m, 3H) 1.58-1.63 (m, 4H), 1.63-1.74 (m, 4H), 2.20 (m, 4H), 2.32 (m, 3H), 2.43- 2.50 (m, 3H), 2.88 (brs, 2H), 3.64 (s, 2H), 3.95(d, J = 7.06 Hz, 2H), 6.96 (t, J = 7.55 Hz, 1H), 7.11 (t, J = 7.03 Hz, 1H), 7.18 (brs, 1H), 7.40(d, J = 8.18 Hz, 1H), 7.58 (d, J = 8.15 Hz, 1H). |
| 304 | 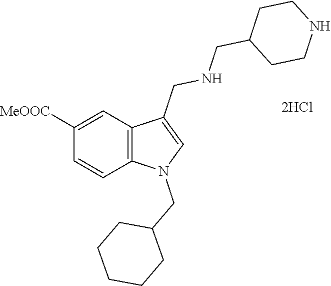 [SMILES: COC(=O)c1ccc2c(c1)c(CNCC1CCNCC1)cn2CC1CCCCC1 ; "2HCl"] | ¹H NMR (400 MHz, DMSO-d₆): δ 0.99-1.10 (m, 2H), 1.12- 1.35 (m, 4H), 1.35-1.44(m, 2H), 1.52 (d, J = 11.77 Hz, 2H), 1.59-1.75 (m, 3H), 1.77-1.89 (m, 1H), 1.92 (d, J = 13.97 Hz, 2H), 2.0-2.32 (m, 1H), 2.79-2.87 (m, 4H), 3.24-3.33 (m, 2H), 3.87 (s, 3H), 4.08(d, J = 7.07 Hz, 2H), 4.34 (brs, 2H), 7.64 (d, J = 8.73 Hz, 1H), 7.79 (s, 1H), 7.80(d, J = 1.20 Hz, 1H), 8.50 (s, 1H), 8.80 (brs, 1H), 8.81-9.11 (m, 3H). |
| 305 | 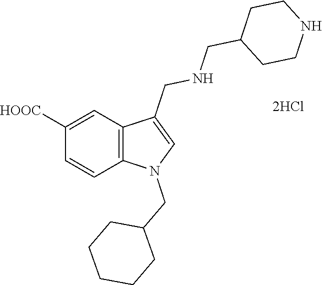 [SMILES: O=C(O)c1ccc2c(c1)c(CNCC1CCNCC1)cn2CC1CCCCC1 ; "2HCl"] | ¹H NMR (400 MHz, DMSO-d₆): δ 0.84-1.01 (m, 2H), 1.09- 1.23 (m, 4H), 1.35-1.44(m, 2H), 1.52 (d, J = 11.45 Hz, 2H), 1.53-1.69 (m, 3H), 1.70-1.83 (m, 2H), 1.92 (d, J = 12.63 Hz, 2H), 2.03 (brs, 1H), 2.79-2.87 (m, 4H), 3.24 (d, J = 12.09 Hz, 2H), 4.06 (d, J = 6.99 Hz, 2H), 4.32 (s, 2H), 7.59 (d, J = 8.73 Hz, 1H), 7.69 (s, 1H), 7.77 (d, J = 8.63 Hz, 1H), 8.45 (s, 1H), 8.97 (brs, 3H), 12.45 (brs, 1H). |
| 306 | 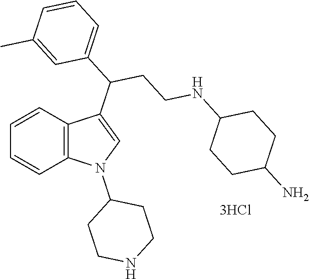 [SMILES: Cc1cccc(C(CCNC2CCC(N)CC2)c2cn(C3CCNCC3)c3ccccc23)c1] | ¹H NMR (400 MHz, DMSO-d₆): δ 1.66-1.85 (m, 9H), 1.92 (brs, 1H), 2.05-2.12 (m, 2H), 2.24 (s, 5H), 2.33-2.40 (m, 1H), 2.75-2.81 (m, 2H), 2.92-2.94 (m, 2H), 3.13 (brs, 5H), 3.43 (d, 2H), 4.23 (t, J = 7.16 Hz, 1H), 4.65-4.67 (m, 1H), 6.95-6.99 (m, 2H), 7.10-7.19 (m, 4H), 7.33 (s, 1H), 7.45 (d, J = 7.91 Hz, 1H), 7.52 (d, J = 8.08 Hz, 1H), 7.95 (brs, 2H), 8.70 (brs, 2H), 8.80-8.98 (m, 3H). |
| 307 | 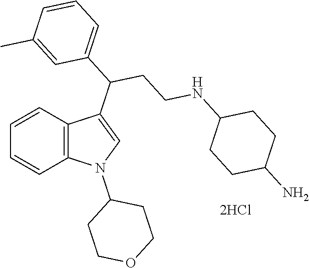 [SMILES: Cc1cccc(C(CCNC2CCC(N)CC2)c2cn(C3CCOCC3)c3ccccc23)c1] | ¹H NMR (400 MHz, DMSO-d₆): δ 1.69-1.97 (m, 10H), 2.0- 2.05 (m, 2H), 2.24 (s, 3H), 2.32-2.35 (m, 2H), 2.56-2.58 (m, 1H), 3.15-3.24 (m, 3H), 3.57 (t, J = 11.18 Hz, 2H), 3.99-4.01 (m, 2H), 4.25 (t, J = 7.40 Hz, 1H), 4.60 (t, J = 10.82 Hz, 1H), 6.92 (t, J = 7.21 Hz, 1H), 6.96 (d, J = 5.96 Hz, 1H), 7.07 (t, J = 7.64 Hz, 1H), 7.14 (s, 3H), 7.39 (d, J = 7.98 Hz, 1H), 7.51 (d, J = 8.03 Hz, 1H), 7.59 (s, 1H), 8.02 (brs, 3H), 8.94 (brs, 1H), 8.99 (brs, 1H). |
| 308 | 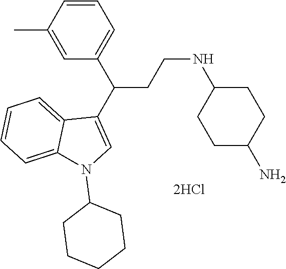 [SMILES: Cc1cccc(C(CCNC2CCC(N)CC2)c2cn(C3CCCCC3)c3ccccc23)c1] | ¹H NMR (400 MHz, DMSO-d₆): δ 1.20-1.32 (m, 1H), 1.52- 1.55 (m, 2H), 1.62-1.90 (m, 13H), 1.95-2.06 (m, 2H), 2.24 (s, 3H), 2.33-2.35 (m, 1H), 2.81(m, 1H), 2.91(m, 1H), 3.16(m, 1H), 3.23 (m, 1H), 4.22-4.32 (m, 2H), 6.90 (t, J = 7.26 Hz, 1H), 6.96 (d, J = 6.14 Hz, 1H), 7.05 (t, J = 7.45 Hz, 1H), 7.14-7.17 (m, 3H), 7.39 (d, J = 7.91 Hz, 1H), 7.45 (d, J = 8.27 Hz, 1H), 7.53 (s, 1H), 8.01 (brs, 3H), 8.93 (brs, 1H), 8.98 (brs, 1H). |
| 309 | 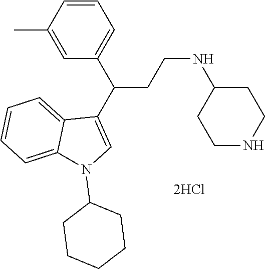 [SMILES: Cc1cccc(C(CCNC2CCNCC2)c2cn(C3CCCCC3)c3ccccc23)c1] | ¹H NMR (400 MHz, DMSO-d₆): δ 1.22-1.25 (m, 1H), 1.46- 1.55 (m, 2H), 1.70-1.83 (m, 8H), 1.94 (m, 2H), 2.095 (d, J = 12.72 Hz, 2H), 2.24 (s, 3H), 2.31-2.49(m, 1H), 2.65-2.89(m, 4H), 3.33(m, 1H), 4.23-4.32 (m, 2H), 6.90 (t, J = 7.28 Hz, 1H), 6.96 (d, J = 6.92 Hz, 1H), 7.06 (t, J = 7.49 Hz, 1H), 7.11-7.17 (m, 3H), 7.39 (d, J = 7.95 Hz, 1H), 7.45 (d, J = 8.30 Hz, 1H), 7.52 (s, 1H), 8.81 (brs, 1H), 8.94 (brs, 1H), 9.32 (brs, 1H). 9.34 (brs, 1H). |
| 310 | 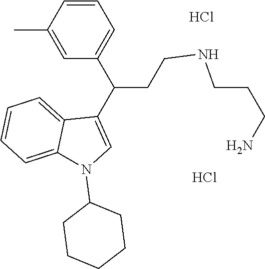 [SMILES: Cc1cccc(C(CCNCCCN)c2cn(C3CCCCC3)c3ccccc23)c1 ; Cl ; Cl] | ¹H NMR (400 MHz, DMSO-d₆): δ 1.22-1.32 (m, 2H), 1.48- 1.52 (m, 2H), 1.73-1.83 (m, 3H), 1.86-1.89 (m, 2H), 1.94- 1.97 (m, 2H), 2.24 (s, 3H), 2.72-2.82 (m, 1H), 2.85-2.86 (m, 2H), 2.95-3.01 (m, 2H), 4.21-4.32(m, 2H), 6.90 (t, J = 7.12 Hz, 1H), 6.97 (d, J = 7.38 Hz, 1H), 7.06 (t, J = 7.40 Hz, 1H), 7.13-7.17 (m, 3H), 7.38 (d, J = 8.02 Hz, 1H), 7.45 (d, J = 9.74 Hz, 2H), 7.86 (brs, 3H), 8.86 (brs, 2H). |
| 311 | 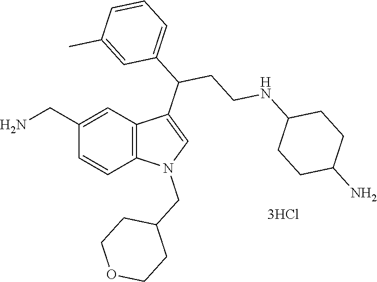 [SMILES: Cc1cccc(C(CCNC2CCC(N)CC2)c2cn(CC3CCOCC3)c3ccc(CN)cc23)c1] | ¹H NMR (400 MHz, DMSO-d₆): δ 9.28 (brs, 1H), 9.17 (brs, 1H), 8.96 (brs, 2H), 8.30 (brs, 3H), 8.12 (brs, 1H), 7.66 (s, 1H), 7.51 (d, J = 8.47 Hz, 1H), 7.46 (s, 1H), 7.21 (d, J = 8.47 Hz, 1H), 7.18-7.14 (m, 3H), 6.97 (d, J = 5.88 Hz, 1H), 4.23- 4.19 (m, 1H), 4.10-4.01 (m, 4H), 3.79 (d, J = 10.73 Hz, 2H), 3.21-3.10 (m, 4H), 2.95-2.84 (m, 2H), 2.72-2.65 (m, 1H), 2.40-2.38 (m, 1H), 2.24 (s, 3H), 2.10-1.98 (m, 3H), 1.90-1.60 [symbol] (m, 6H), 1.85-1.20 (m, 4H) |
| 312 | 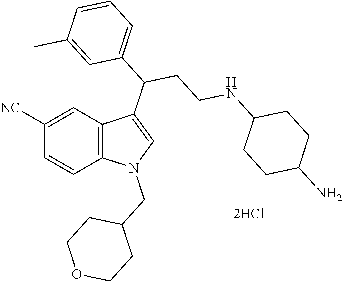 [SMILES: Cc1cccc(C(CCNC2CCC(N)CC2)c2cn(CC3CCOCC3)c3ccc(C#N)cc23)c1] | ¹H NMR (400 MHz, DMSO-d₆): δ 8.97 (brs, 2H), 9.17 (brs, 1H), 8.01 (brs, 3H), 7.94 (s, 1H), 7.69 (d, J = 8.73 Hz, 1H), 7.65 (s, 1H), 7.44 (d, J = 8.54 Hz, 1H), 7.20-7.15 (m, 3H), 6.99 (d, J = 5.73 Hz, 1H), 4.36-4.33 (m, 1H), 4.15-4.10 (m, 2H), 3.80 (d, J = 10.23 Hz, 2H), 3.22-3.16 (m, 4H), 2.89 (brs [symbol] 1H), 2.80 (brs, 1H), 2.36-2.34 (m, 1H), 2.24 (s, 3H), 2.10-1.9 [symbol] (m, 1H), 1.90-1.60 (m, 8H), 1.41-1.20 (m, 4H). |
| 313 | 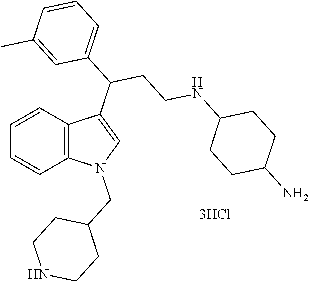 [SMILES: Cc1cccc(C(CCNC2CCC(N)CC2)c2cn(CC3CCNCC3)c3ccccc23)c1] | ¹H NMR (400 MHz, DMSO-d₆): δ 1.40-1.46 (m, 2H), 1.48- 1.52 (m, 2H), 1.62-1.84 (m, 10H), 2.11 (m, 1H), 2.24 (s, 3H), 2.31-2.34 (m, 1H), 2.73-2.82 (m, 3H), 2.93 (m, 1H), 3.14 (m, 1H), 3.23(d, J = 11.9 Hz, 3H), 4.08(d, J = 6.85 Hz, 2H), 4.25(t, 4H), 6.92 (t, J = 7.24 Hz, 1H), 6.96 (d, J = 7.30 Hz, 1H), 7.06-7.17 (m, J = 7.40 Hz, 4H), 7.38-7.45 (m, 3H), 8.05 (brs, 1H), 8.60 (brs, 1H), 8.79 (brs, 1H), 9.02 (brs, 1H), 9.11 (brs, 1H). |
| 314 | 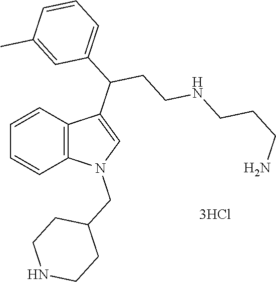 [SMILES: Cc1cccc(C(CCNCCCN)c2cn(CC3CCNCC3)c3ccccc23)c1] | ¹H NMR (400 MHz, DMSO-d₆): δ 1.40-1.43 (m, 2H), 1.62- 1.63 (m, 2H), 1.92-2.01 (m, 2H), 2.24 (s, 3H), 2.77-3.94 (m, 8H), 3.23 (d, J = 11.51 Hz, 2H), 4.08 (d, J = 6.41 Hz, 2H), 4.25 (t, J = 7.73 Hz, 1H), 6.92 (t, J = 7.34 Hz, 1H), 6.97 (d, J = 7.32 Hz, 1H), 7.06-7.18 (m, 4H), 7.38-7.45 (m, 3H), 7.99 (brs, 3H), 8.56 (brs, 1H), 8.77 (brs, 1H), 9.13 (brs, 1H), 9.14 (brs, 1H). |
| 315 | 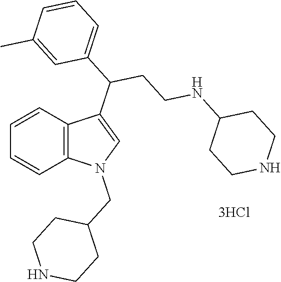 [SMILES: Cc1cccc(C(CCNC2CCNCC2)c2cn(CC3CCNCC3)c3ccccc23)c1] | ¹H NMR (400 MHz, DMSO-d₆): δ 1.40-1.43 (m, 2H), 1.64 (d, J = 13.71 Hz, 2H), 1.82-1.92 (m, 2H), 2.12 (d, J = 12.92 Hz, 3H), 2.24 (s, 3H), 2.31-2.34 (m, 1H), 2.80-2.89 (m, 7H), 3.23 (d, J = 11.20 Hz, 3H), 4.08 (d, J = 6.81 Hz, 1H), 4.27 (t, J = 7.84 Hz, 1H), 6.90-6.98 (m, 2H), 7.07-7.18 (m, 4H), 7.39-7.45 (m, 3H), 8.61 (brs, 1H), 8.81 (brs, 1H), 8.96 (brs, 1H), 9.02 (brs, 1H), 9.43 (brs, 1H), 9.46 (brs, 1H). |
| 316 | 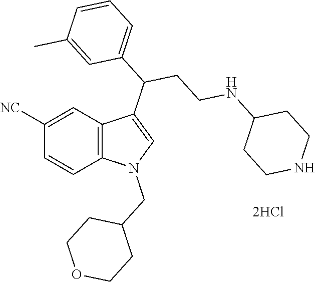 [SMILES: Cc1cccc(C(CCNC2CCNCC2)c2cn(CC3CCOCC3)c3ccc(C#N)cc23)c1] | ¹H NMR (400 MHz, DMSO-d₆): δ 9.44-9.32 (m, 2H), 8.91- 8.75 (m, 2H), 7.95 (s, 1H), 7.70 (d, J = 8.51 Hz, 1H), 7.62 (s, 1H), 7.45 (d, J = 8.50 Hz, 1H), 7.21-7.16 (m, 3H), 7.00 (d, J = [symbol] 6.54 Hz, 1H), 4.36(t, J = 7.55 Hz, 1H), 4.11 (d, J = 4.76 Hz, 2H), 3.80 (d, J = 10.25 Hz, 2H), 3.33- 3.20 (m, 4H), 2.66-2.8 [symbol] (m, 5H), 2.35-2.32 (m, 1H), 2.25 (s, 3H), 2.15-2.05 (m, 3H), 1.85-1.72 (m, 2H), 1.39-1.23 (m, 4H). |
| 317 | 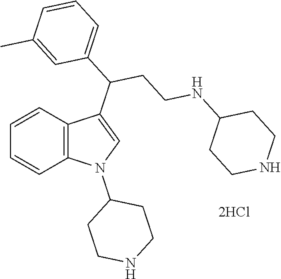 [SMILES: Cc1cccc(C(CCNC2CCNCC2)c2cn(C3CCNCC3)c3ccccc23)c1] | ¹H NMR (400 MHz, DMSO-d₆): δ 1.79-1.85 (m, 2H), 2.10 (m, 4H), 2.24 (s, 3H), 2.29-2.40 (m, 3H), 2.55 (m, 1H), 2.72-2.89 (m, 4H), 3.11-3.13(m, 2H), 3.31-3.47 (m, 11H), 4.29 (t, J = 8.0 Hz, 1H), 4.68 (t, J = 11.80 Hz, 1H), 6.93- 6.98 (m, 2H), 7.09-7.19 (m, 4H), 7.35 (s, 1H), 7.45(d, J = 7.19 Hz, 1H), 7.54 (d, J = 8.28 Hz, 1H), 8.87-8.89 (m, 1H), 9.10 (brs, 1H), 9.49 (brs, 1H), 9.55(brs, 1H). |
| 318 | 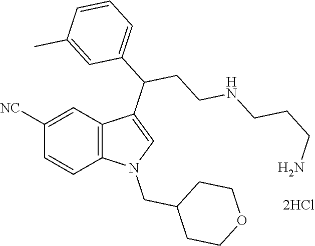 [SMILES: Cc1cccc(C(CCNCCCN)c2cn(CC3CCOCC3)c3ccc(C#N)cc23)c1] | ¹H NMR (400 MHz, DMSO-d₆): δ 1.22-1.37 (m, 5H), 1.87- 1.90 (m, 2H), 2.00-2.02 (m, 1H), 2.24 (s, 3H), 2.72-2.74 (m, 1H), 2.85-2.90 (m, 3H), 2.94-2.96 (m, 2H), 3.20 (t, J = 10.39 Hz, 2H), 3.81 (d, J = 11.28 Hz, 2H), 4.11(d, J = 6.92 Hz, 2H), 4.34 (t, J = 7.91 Hz, 1H), 7.0 (d, J = 6.8 Hz, 1H), 7.15-7.21 (m, 3H), 7.44 (d, J = 8.53 Hz, 1H), 7.58(s, 1H), 7.69 (d, J = 8.56 Hz, 1H), 7.90 (brs, 3H), 7.92 (s, 1H), 8.96 (brs, 1H), 8.99 (brs, 1H). |
| 319 | 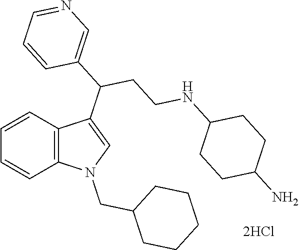 [SMILES: NC1CCC(NCCC(c2cccnc2)c2cn(CC3CCCCC3)c3ccccc23)CC1] | ¹H NMR (400 MHz, DMSO-d₆): δ 0.96-1.12 (m, 5H), 1.50 (d, J = 11.11 Hz, 2H), 1.59-1.83 (m, 12H), 2.61-2.63 (m, 1H), 2.81-2.98 (m, 3H), 3.15-3.32 (m, 2H), 3.93-4.02 (m, 2H), 4.65 (t, J = 7.13 Hz, 1H), 6.94 (t, J = 7.62 Hz, 1H), 7.09 (t, J = 7.69 Hz, 1H), 7.45 (d, J = 8.17 Hz, 2H), 7.53 (s, 1H), 7.77 (m, 1H), 8.08 (brs, 3H), 8.27 (brs, 1H), 8.65 (d, J = 4.63 Hz, 1H), 8.87 (brs, 1H), 9.17 (brs, 1H), 9.25 (brs, 1H). |
| 320 | 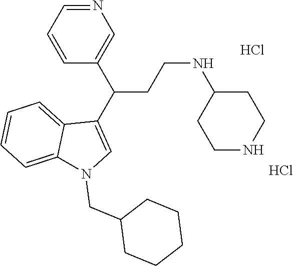 [SMILES: c1cncc(C(CCNC2CCNCC2)c2cn(CC3CCCCC3)c3ccccc23)c1 ; Cl ; Cl] | ¹H NMR (400 MHz, DMSO-d₆): δ 0.96-1.12 (m, 5H), 1.50 (d, J = 12.17 Hz, 3H), 1.59-1.64 (m, 3H), 2.13 (d, J = 12.79 Hz, 2H), 2.52-2.59 (m, 1H), 2.71-2.89 (m, 3H), 2.90-2.99 (m, 2H), 3.35-3.49 (m, 3H), 3.97-3.98 (m, 2H), 4.65 (t, J = 7.13 Hz, 1H), 6.94 (t, J = 7.16 Hz, 1H), 7.10 (t, J = 7.18 Hz, 1H), 7.40-7.44 (m, 2H), 7.51 (s, 1H), 7.78 (s, 1H), 8.27 (s, 1H), 8.66 (d, J = 4.46 Hz, 1H), 8.87 (brs, 2H), 9.05 (brs, 1H), 9.55 (brs, 1H), 9.66 (brs, 1H). |
| 321 | 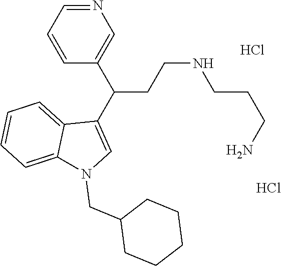 [SMILES: NCCCNCCC(c1cccnc1)c1cn(CC2CCCCC2)c2ccccc12 ; Cl ; Cl] | ¹H NMR (400 MHz, DMSO-d₆): δ 0.99-1.49 (m, 7H), 1.60- 1.80 (m, 6H), 1.97 (m, 2H), 2.89-2.95 (m, 6H), 3.98 (s, 2H), 4.78 (s, 1H), 6.93 (s, 1H), 7.09 (s, 1H), 7.42-7.51 (m, 3H), 7.82 (s, 1H), 8.10 (brs, 3H), 8.33 (bs, 1H), 8.68 (s, 1H), 8.90 (brs, 1H), 9.40 (brs, 1H), 9.50 (brs, 1H). |
| 322 | 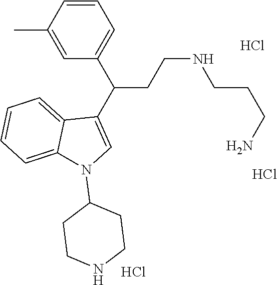 [SMILES: Cc1cccc(C(CCNCCCN)c2cn(C3CCNCC3)c3ccccc23)c1 ; Cl ; Cl ; Cl] | ¹H NMR (400 MHz, DMSO-d₆): δ 1.91-1.94 (m, 2H), 2.10 (d, J = 10.37 Hz, 2H), 2.25-2.31 (m, 5H), 2.34-2.40 (m, 1H), 2.87-2.95 (m, 5H), 3.12 (s, 2H), 3.42 (d, J = 12.00 Hz, 2H), 4.27 (t, J = 7.40 Hz, 1H), 4.68 (t, J = 11.80 Hz, 1H), 6.93-6.98 (m, 2H), 7.09-7.19 (m, 4H), 7.34 (s, 1H), 7.44 (d, J = 7.97 Hz, 1H), 7.54 (d, J = 8.16 Hz, 1H), 8.02 (brs, 3H), 9.06 (brs, 1H), 9.17 (brs, 1H), 9.20 (brs, 1H), 9.26 (brs, 1H). |
| 323 | 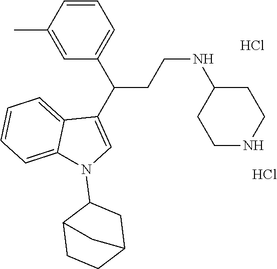 [SMILES: Cc1cccc(C(CCNC2CCNCC2)c2cn(C3CC4CCC3C4)c3ccccc23)c1 ; Cl ; Cl] | ¹H NMR (400 MHz, DMSO-d₆): δ 0.94 (s, 1H), 1.23-1.26 (m, 1H), 1.40 (d, J = 7.69 Hz, 1H), 1.57-1.59 (m, 3H), 1.79-1.89 (m, 3H), 2.11-2.16 (m, 3H), 2.24(s, 3H), 2.35(d, J = 23.61 Hz, 1H), 4.29-4.31 (m, 1H), 4.88 (s, 1H), 6.90-6.97 (m, 2H), 7.07 (t, J = 7.79 Hz, 1H), 7.11-7.17 (m, 3H), 7.37- 7.40 (m, 1H), 7.44 (d, J = 8.23 Hz, 1H), 7.60-7.66 (m, 1H), 8.86 (brs, 1H), 9.23 (brs, 1H), 9.36 (brs, 1H), 9.40 (brs, 1H). |
| 324 | 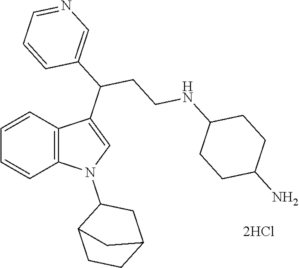 [SMILES: NC1CCC(NCCC(c2cccnc2)c2cn(C3CC4CCC3C4)c3ccccc23)CC1] | ¹H NMR (400 MHz, DMSO-d₆): δ 0.92 (s, 1H), 1.22 (t, J = 10.85 Hz, 1H), 1.40 (d, J = 9.03 Hz, 1H), 1.55 (s, 3H), 1.65- 1.96 (m, 12H), 2.14 (t, J = 11.05 Hz, 1H), 2.65 (s, 1H), 2.78-2.95 (m, 4H), 3.16-3.22 (m, 2H), 4.71 (t, J = 8.09 Hz, 1H), 4.88 (d, J = 5.65 Hz, 1H), 6.96 (t, J = 7.54 Hz, 1H), 7.10 (t, J = 7.62 Hz, 1H), 7.42-7.48 (m, 2H), 7.83 (t, J = 7.12 Hz, 1H), 7.87 (s, 1H), 8.10 (brs, 3H), 8.37 (d, J = 7.41 Hz, 1H), 8.67(d, J = 5.07 Hz, 1H), 8.95 (s, 1H), 9.24 (brs, 1H), 9.44 (brs, 1H). |
| 325 | 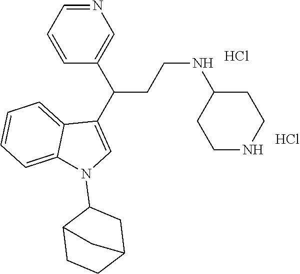 [SMILES: c1cncc(C(CCNC2CCNCC2)c2cn(C3CC4CCC3C4)c3ccccc23)c1 ; Cl ; Cl] | ¹H NMR (400 MHz, DMSO-d₆): δ 0.92-0.97 (m, 1H), 1.23 (m, 1H), 1.40 (d J = 8.80 Hz, 1H), 1.53-1.57 (m 1H), 1.66- 1.68 (m, 2H), 1.85-1.95 (m, 3H), 2.15 (s, 1H), 2.38 (s, 1H), 2.57 (s, 1H), 2.72-2.74 (m, 1H), 2.87-3.05 (m, 4H), 3.26- 3.41 (m, 4H), 4.74 (t, J = 6.5 Hz, 1H), 4.88 (d, J = 11.51 Hz, 1H) 6.93 (t, J = 7.45 Hz, 1H), 7.11 (t, J = 7.56 Hz, 1H), 7.43-7.49 (m, 2H), 7.82-7.87 (m, 2H), 8.38 (d, J = 6.93 Hz, 1H), 8.86-8.90 (m, 1H), 8.96 (s, 1H), 9.08 (s, 1H), 9.08 (s, 1H), 9.61 (brs, 1H), 9.82 (brs, 1H). |
| 326 | 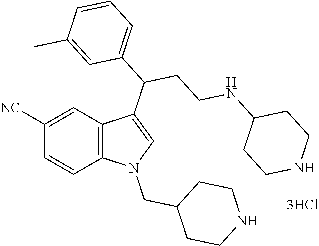 [SMILES: Cc1cccc(C(CCNC2CCNCC2)c2cn(CC3CCNCC3)c3ccc(C#N)cc23)c1] | ¹H NMR (400 MHz, DMSO-d6) δ 9.65 (brs, 1H), 9.55 (brs, 1H), 9.11 (brs, 1H), 8.89 (brs, 2H), 8.72-8.70 (m, 1H), 7.96 (s [symbol] 1H), 7.70-7.69 (m, 2H), 7.47 (d, J = 8.34 Hz, 1H), 7.19-7.17 (m, 3H), 7.01-6.99 (m, 1H), 4.38 (t, J = 7.42 Hz, 1H), 4.16 (d, J = 6.49 Hz, 2H), 3.33-3.32 (m, 1H), 3.22 (d, J = 12.35 Hz [symbol] 2H), 2.86-2.66 (m, 6H), 2.40- 2.32 (m, 1H), 2.26 (s, 3H), 2.15 [symbol] 2.11 (m, 3H), 1.84- 1.81(m, 2H), 1.64-1.62 (m, 2H), 1.46-1.38 [symbol] (m, 2H). |
| 327 | 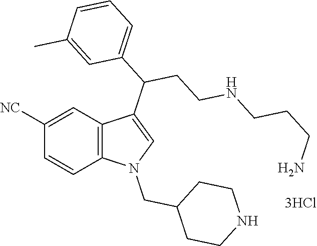 [SMILES: Cc1cccc(C(CCNCCCN)c2cn(CC3CCNCC3)c3ccc(C#N)cc23)c1] | ¹H NMR (400 MHz, DMSO-d6) δ 9.33 (brs, 1H), 9.25 (brs, 1H), 8.86 (brs, 1H), 8.66 (brs, 1H), 8.03 (s, 3H), 7.94 (s, 1H), 7.69-7.65 (m, 2H), 7.46(d, J = 8.38 Hz, 1H), 7.21-7.16 (m, 3H), 7.0 (d, J = 6.33 Hz, 1H), 4.36 (t, J = 7.64 Hz, 1H), 4.15 (d, J = 6.8 Hz, 2H), 3.22 (d, J = 10.97 Hz, 2H), 2.95-2.76 (m, 9H), 2.36-2.34 (m, 1H), 2.25 (s, 3H), 2.13-2.11 (m, 1H), 1.91 [symbol] 1.95 (m, 2H), 1.64-1.59 (m, 2H), 1.46-1.40 (m, 2H). |
| 328 | 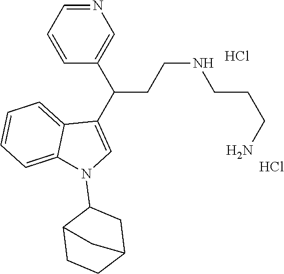 [SMILES: NCCCNCCC(c1cccnc1)c1cn(C2CC3CCC2C3)c2ccccc12 ; Cl ; Cl] | ¹H NMR (400 MHz, DMSO-d₆): δ 0.94 (m, 1H), 1.40 (d, J = 9.68 Hz, 1H), 1.53 (s, 1H), 1.68 (d, J = 8.65 Hz, 2H), 1.92 (s, 3H), 2.14 (s, 1H), 2.38 (s, 1H), 2.57 (s, 1H), 2.66 (s, 1H), 2.87-2.96 (m, 7H), 4.65 (s, 1H), 4.87 (t, J = 5.11 Hz, 1H), 6.96 (t, J = 7.35 Hz, 1H), 7.10 (t, J = 7.48 Hz, 1H), 7.46 (t, J = 10.20 Hz, 2H), 7.72 (brs, 1H), 7.77 (brs, 1H), 7.98 (brs, 3H), 8.26 (brs, 1H), 8.60 (brs, 1H), 8.90 (brs, 1H), 9.26 (brs, 1H), 9.37 (brs, 1H). |
| 329 | 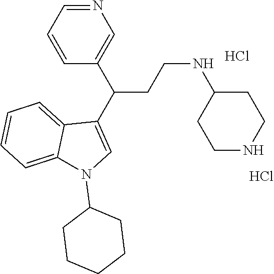 [SMILES: c1cncc(C(CCNC2CCNCC2)c2cn(C3CCCCC3)c3ccccc23)c1 ; Cl ; Cl] | ¹H NMR (400 MHz, DMSO-d₆): δ 1.25-1.39 (m, 1H), 1.49- 1.52 (m, 2H), 1.74-1.86 (m, 7H), 1.95 (d, J = 10.62 Hz, 2H), 2.13 (d, J = 12.81 Hz, 2H), 2.65 (s, 1H), 2.83-2.86 (m, 5H), 3.32-3.37 (m, 5H), 4.31(t, J = 11.87 Hz, 1H), 4.65 (t, J = 7.12 Hz, 1H), 6.94 (t, J = 7.66 Hz, 1H), 7.09 (t, J = 7.82 Hz, 1H), 7.47 (dd, J_{1,2} = 8.0 Hz, J_{1,3} = 16.40 Hz, 2H), 7.69 (brs, 1H), 7.77 (brs, 1H), 8.27 (brs, 3H), 8.64 (d, J = 4.60 Hz, 2H), 8.82 (brs, 1H), 8.89 (brs, 1H), 8.97 (s, 1H), 9.51 (brs, 1H), 9.69 (1H). |
| 330 | 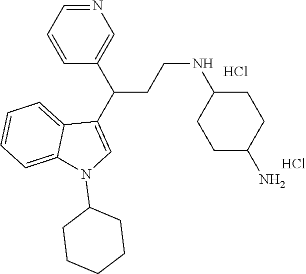 [SMILES: NC1CCC(NCCC(c2cccnc2)c2cn(C3CCCCC3)c3ccccc23)CC1 ; Cl ; Cl] | ¹H NMR (400 MHz, DMSO-d₆): δ 1.22-1.52 (m, 8H), 1.71- 1.74 (m, 3H), 1.85 (d, J = 13.89 Hz, 2H), 2.03 (s, 1H), 2.49-2.50 (m, 1H), 2.83-2.94 (m, 5H), 4.31 (t, J = 11.15 Hz, 1H), 4.31(t, J = 11.15 Hz, 1H), 4.34 (m, 1H), 6.94 (t, J = 7.43 Hz, 1H), 7.09 (t, J = 7.42 Hz, 1H), 7.46 (dd, J_{1,2} = 7.88 Hz, J_{1,3} = 18.29 Hz, 2H), 7.61-7.65 (m, 1H), 7.99 (brs, 1H), 8.58 (brs, 1H), 8.84 (s, 1H), 9.12 (brs, 1H), 9.27 (1H). |
| 331 | 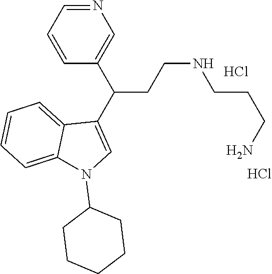 [SMILES: NCCCNCCC(c1cccnc1)c1cn(C2CCCCC2)c2ccccc12 ; Cl ; Cl] | ¹H NMR (400 MHz, DMSO-d₆): δ 1.22-1.31 (m, 2H), 1.48- 1.52 (m, 2H), 1.55-1.86 (m, 5H), 2.49-2.59 (m, 1H), 2.87- 2.96 (m, 7H), 4.31 (t, J = 10.54 Hz, 1H), 4.66 (s, 1H), 6.94 (t, J = 7.20 Hz, 1H), 7.09 (t, J = 8.03 Hz, 1H), 7.47 (dd, J_{1,2} = 7.78 Hz, J_{1,3} = 13. 0 Hz, 2H), 7.67 (s, 1H), 7.78 (brs, 1H), 8.01 (brs, 1H), 8.30 (brs, 1H), 8.65 (brs, 1H), 8.90 (brs, 1H), 9.27 (1H), 9.38 (brs, 1H). |
| 332 | 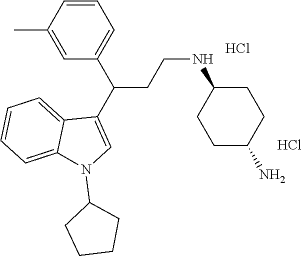 [SMILES: Cc1cccc(C(CCN[C@H]2CC[C@H](N)CC2)c2cn(C3CCCC3)c3ccccc23)c1 ; Cl ; Cl] | ¹H NMR (400 MHz, DMSO-d₆): δ 9.32 (brs, 1H), 9.15 (brs, 1H), 8.10 (s, 3H), 7.52 (s, 1H), 7.44 (s, 1H), 7.43 (d, J = 8.21 Hz, 1H), 7.38 (d, J = 7.82 Hz, 1H), 7.16-7.13 (m, 3H), 7.06 (t [symbol] J = 7.59 Hz, 1H), 6.96-6.94 (m, 1H), 6.90 (t, J = 7.55 Hz, 1H [symbol] 4.86-4.83 (m, 1H), 4.25 (t, J = 7.35 Hz, 1H), 3.0-2.91 (m, 3H), 2.81-2.73 (m, 1H), 2.60-2.55 (m, 1H), 2.35-2.30 (m, 1H [symbol] 2.23 (s, 3H), 1.87-1.70 (m, 2H), 1.44-1.29 (m, 4H). |
| 333 | 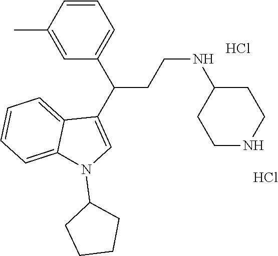 [SMILES: Cc1cccc(C(CCNC2CCNCC2)c2cn(C3CCCC3)c3ccccc23)c1 ; Cl ; Cl] | ¹H NMR (400 MHz, DMSO-d₆): δ 9.57 (brs, 1H), 9.43 (brs, 1H), 9.04-9.10 (m, 1H), 8.90-8.82 (m, 1H), 7.52 (s, 1H), 7.43 (d, J = 8.28 Hz, 1H), 7.39 (d, J = 7.88 Hz, 1H), 7.17-7.12 (m, 3H), 7.06 (t, J = 7.57 Hz, 1H), 6.96-6.94 (m, 1H), 6.90 (t, J = 7.53 Hz, 1H), 4.85 (t, J = 7.05 Hz, 1H), 4.26 (t, J = 7.87 Hz, 1H), 3.42-3.30 (m, 3H), 2.89-2.80 (m, 4H), 2.59-2.53 (m, 1H [symbol] 2.38-2.30 (m, 1H), 2.24 (s, 3H), 2.18-2.10 (m, 4H), 1.92-1.80 [symbol] (m, 6H), 1.72-1.69 (m, 2H). |
| 334 | 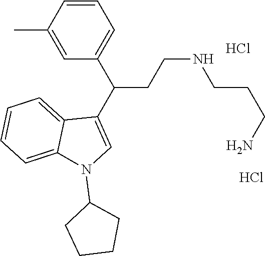 [SMILES: Cc1cccc(C(CCNCCCN)c2cn(C3CCCC3)c3ccccc23)c1 ; Cl ; Cl] | ¹H NMR (400 MHz, DMSO-d₆): δ 9.16 (brs, 1H), 8.10 (brs, 1H), 7.94 (s, 1H), 7.43 (d, J = 8.26 Hz, 1H), 7.37 (d, J = 7.92 Hz, 1H), 7.13-7.17 (m, 3H), 7.06 (t, J = 7.61 Hz, 1H), 6.95 (d [symbol] J = 6.69 Hz, 1H), 6.90 (t, J = 7.44 Hz, 1H), 4.84 (t, J = 6.82 Hz, 1H), 4.24 (t, J = 7.55 Hz, 1H), 2.94 (t, J = 7.17 Hz, 2H), 2.87(t, J = 7.33 Hz, 2H), 2.79-2.72 (m, 1H), 2.38-2.32 (m, 1H), 2.24 (s, 3H), 2.19-2.10 (m, 2H), 1.98- 1.82 (m, 6H), 1.75 [symbol] 1.66 (m, 2H). |
| 335 | 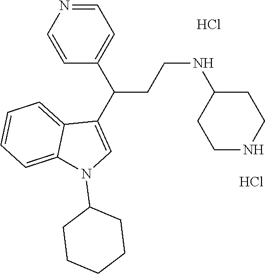 [SMILES: c1ccc2c(c1)c(C(CCNC1CCNCC1)c1ccncc1)cn2C1CCCCC1 ; Cl ; Cl] | ¹H NMR (400 MHz, DMSO-d₆): δ 1.22-1.31 (m, 1H), 1.46- 1.52 (m, 2H), 1.70-1.85 (m, 7H), 1.96 (d, J = 11.08 Hz, 2H), 2.14 (d, J = 11.06 Hz, 2H), 2.65 (s, 1H), 3.32-3.37(m, 4H), 4.32 (t, J = 11.18 Hz, 1H), 4.76 (t, J = 6.37 Hz, 1H), 6.95 (t, J = 7.41 Hz, 1H), 7.10 (t, J = 7.78 Hz, 1H), 7.47 (dd, J_{1,2} = 7.78 Hz, J_{1,3} = 21.01 Hz, 2H), 7.78 (s, 1H), 7.94 (d, J = 4.67 Hz, 2H), 8.75 (d, J = 5.97 Hz, 2H), 8.82 (brs, 1H), 9.06 (brs, 1H), 9.66 (brs, 1H), 9.81 (brs, 1H). |
| 336 | 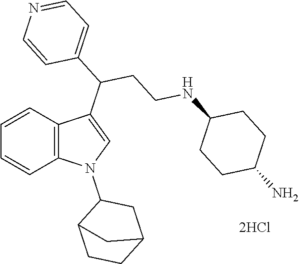 [SMILES: N[C@H]1CC[C@H](NCCC(c2ccncc2)c2cn(C3CC4CCC3C4)c3ccccc23)CC1] | ¹H NMR (400 MHz, DMSO-d₆): δ 0.87-0.91 (m, 1H), 1.22- 1.25 (m, 1H), 1.32-1.55 (m, 6H), 1.65-1.74 (m, 2H), 1.98 (d, J = 11.59 Hz, 3H), 2.07-2.17 (m, 3H), 2.37 (s, 1H), 2.65 (s, 1H), 2.71-2.80 (m, 1H), 2.91 (brs, 4H), 4.84 (t, J = 6.69 Hz, 1H), 4.88-4.89 (m, 1H), 6.94-6.98 (m, 1H), 7.10 (t, J = 7.35 Hz, 1H), 7.40-7.49 (m, 2H), 7.94 (dd, J_{1,2} = 6.788 Hz, J_{1,3} = 14.01 Hz, 2H), 8.0 (d, J = 5.74 Hz, 1H), 8.12 (s, 3H), 8.75-8.78 (m, 2H), 9.45 (brs, 1H), 9.68 (brs, 1H). |
| 337 | 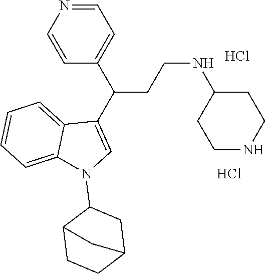 [SMILES: c1ccc2c(c1)c(C(CCNC1CCNCC1)c1ccncc1)cn2C1CC2CCC1C2 ; Cl ; Cl] | ¹H NMR (400 MHz, DMSO-d₆): δ 0.87-0.91 (m, 1H), 1.22 (t, J = 10.76 Hz, 1H), 1.40 (d, J = 9.18 Hz, 1H), 1.55 (brs, 1H), 1.66 (d, J = 9.70 Hz, 1H), 1.70-1.75 (m, 1H), 1.84-2.01 (m, 4H), 2.14 (brs, 3H), 2.37 (s, 1H), 2.48-2.57(m, 2H), 2.74-2.94 (m, 6H), 3.31-3.37 (m, 4H), 4.79-4.90 (m, 2H), 6.97 (t, J = 7.45 Hz, 1H), 7.11 (t, J = 7.32 Hz, 1H), 7.41- 7.49 (m, 2H), 7.91 (s, 1H), 7.95 (d, J = 5.74 Hz, 5.75, 1H), 8.0 (d, J = 6.31 Hz, 1H), 8.76-8.85 (m, 2H), 8.87 (brs, 1H), 9.12 (brs, 1H), 9.73 (brs, 1H), 9.91 (brs, 1H). |
| 338 | 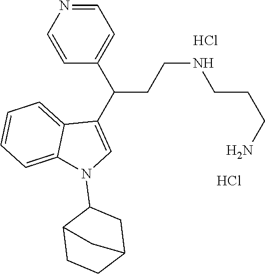 [SMILES: NCCCNCCC(c1ccncc1)c1cn(C2CC3CCC2C3)c2ccccc12 ; Cl ; Cl] | ¹H NMR (400 MHz, DMSO-d₆): δ 0.91 (brs, 1H), 1.22 (m, 1H), 1.40 (d, J = 9.30 Hz, 1H), 1.57 (brs, 1H), 1.66 (d, J = 9.54 Hz, 2H), 1.94-1.96 (m, 2H), 2.14 (t, J = 11.25 Hz, 1H), 2.37 (s, 1H), 2.55-2.59 (m, 1H), 2.70-2.74 (m, 1H), 2.88-2.98 (m, 7H), 4.75-4.81 (m, 1H), 4.88-4.89 (m, 1H), 6.97 (t, J = 7.64 Hz, 1H), 7.10 (t, J = 7.37 Hz, 1H), 7.42- 7.49 (m, 2H), 7.87 (d, J = 8.37 Hz, 1H), 7.92 (d, J = 5.58 Hz, 1H), 7.97 (d, J = 5.76 Hz, 1H), 8.04 (brs, 1H), 8.75 (t, J = 6.19 Hz, 2H), 9.41 (brs, 1H), 9.55 (brs, 1H). |
| 339 | 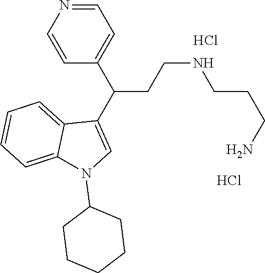 [SMILES: NCCCNCCC(c1ccncc1)c1cn(C2CCCCC2)c2ccccc12 ; Cl ; Cl] | ¹H NMR (400 MHz, DMSO-d₆): δ 1.23-1.26 (m, 1H), 1.49- 1.52 (m, 2H), 1.70-1.87 (m, 5H), 1.94-1.97 (m, 4H), 2.54- 2.62 (m, 1H), 2.88-2.98 (m, 7H), 4.29-4.35 (m, 1H), 4.74- 4.76 (m, 1H), 6.95 (t, J = 7.28 Hz, 1H), 7.10 (t, J = 7.43 Hz, 1H), 7.44 (d, J = 7.97 Hz, 1H), 7.50 (d, J = 8.38 Hz, 1H), 7.73 (s, 1H), 7.94 (brs, 2H), 8.03 (brs, 3H), 8.75 (d, J = 5.24 Hz, 1H), 9.38 (brs, 1H), 9.47 (brs, 1H). |
| 340 | 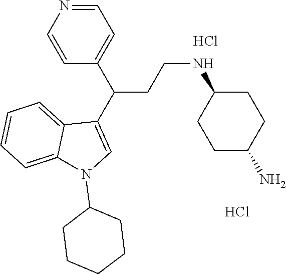 [SMILES: N[C@H]1CC[C@H](NCCC(c2ccncc2)c2cn(C3CCCCC3)c3ccccc23)CC1 ; Cl ; Cl] | ¹H NMR (400 MHz, DMSO-d₆): δ 1.22-1.38 (m, 3H), 1.46- 1.52 (m, 4H), 1.55-1.84 (m, 5H), 1.86-1.97 (m, 4H), 2.06 (s, 1H), 2.45 (m, 1H), 2.60-2.64 (m, 1H), 2.91 (brs, 4H), 4.32 (t, J = 11.27 Hz, 1H), 4.75-4.77 (m, 1H), 6.95 (t, J = 7.40 Hz, 1H), 7.10 (t, J = 7.63 Hz, 1H), 7.44 (d, J = 7.95 Hz, 1H), 7.50 (d, J = 8.36 Hz, 1H), 7.76 (s, 1H), 7.97 (brs, 2H), 8.09 (brs, 3H), 8.76 (d, J = 5.74 Hz, 1H), 9.38 (brs, 1H), 9.53 (brs, 1H). |
| 341 | 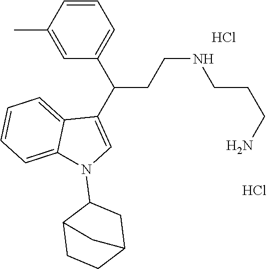 [SMILES: Cc1cccc(C(CCNCCCN)c2cn(C3CC4CCC3C4)c3ccccc23)c1 ; Cl ; Cl] | ¹H NMR (400 MHz, DMSO-d₆): δ 0.91-0.96 (m, 1H), 1.22- 1.32 (m, 2H), 1.39 (d, J = 9.30 Hz, 1H), 1.56-1.58 (m, 1H), 1.66 (d, J = 9.01 Hz, 2H), 1.85-1.97 (m, 3H), 2.12-2.18 (m, 1H), 2.23 (s, 3H), 2.43-2.49 (m, 2H), 2.60-2.65 (m, 2H), 2.87-2.95 (m, 7H), 4.28 (t, J = 7.79 Hz, 1H), 4.84-4.89 (m, 1H), 6.91 (t, J = 7.49 Hz, 1H), 6.96 (d, J = 6.89 Hz, 1H), 7.06 (t, J = 7.81 Hz, 1H), 7.12-7.14 (m, 3H), 7.37 (d, J = 7.96 Hz, 1H), 7.43 (d, J = 8.28 Hz, 1H), 7.61 (s, 1H), 8.02 (brs, 3H), 9.19-9.36 (m, 2H). |
| 342 | 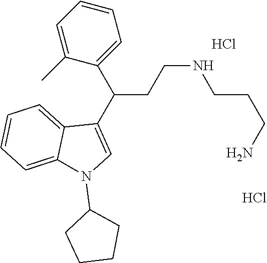 [SMILES: Cc1ccccc1C(CCNCCCN)c1cn(C2CCCC2)c2ccccc12 ; Cl ; Cl] | ¹H NMR (400 MHz, DMSO-d₆): δ 9.19 (brs, 1H), 8.0 (brs, 1H), 7.45-7.42 (m, 2H), 7.30 (d, J = 7.89 Hz, 1H), 7.19 (d, J = [symbol] 7.29 Hz, 1H), 7.14 (d, J = 6.95 Hz, 1H), 7.11-7.04 (m, 3H), 6.89 (t, J = 7.43 Hz, 1H), 4.84 (t, J = 6.59 Hz, 1H), 4.44 (t, J = [symbol] 7.41 Hz, 1H), 2.99-2.82 (m, 6H), 2.40 (s, 3H), 2.25-2.30 (s, 1H), 2.19-2.10 (m, 2H), 1.96-1.80 (m, 6H), 1.72-1.63 (m, 2H) [symbol] 1.22 (s, 1H). |
| 343 | 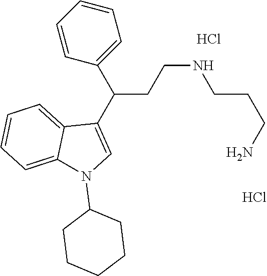 [SMILES: NCCCNCCC(c1ccccc1)c1cn(C2CCCCC2)c2ccccc12 ; Cl ; Cl] | ¹H NMR (400 MHz, DMSO-d₆): δ 1.25-1.32 (m, 1H), 1.45- 1.56 (m, 2H), 1.69-1.89 (m, 5H), 1.91-1.97 (m, 3H), 2.31- 2.37 (m, 1H), 2.76 (brs, 1H), 2.85-2.96 (m, 5H), 4.26-4.32 (m, 2H), 6.89 (t, J = 7.35 Hz, 1H), 7.05 (d, J = 7.30 Hz, 1H), 7.14 (t, J = 7.23 Hz, 1H), 7.26 (t, J = 7.43 Hz, 2H), 7.32-7.38 (m, 2H), 7.45 (d, J = 8.31 Hz, 1H), 7.53 (s, 1H), 8.03 (brs, 3H), 9.21 (brs, 1H), 9.27 (brs, 1H). |
| 344 | 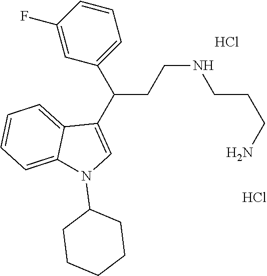 [SMILES: NCCCNCCC(c1cccc(F)c1)c1cn(C2CCCCC2)c2ccccc12 ; Cl ; Cl] | ¹H NMR (400 MHz, DMSO-d₆): δ 1.25-1.32 (m, 1H), 1.46- 1.54 (m, 2H), 1.70-1.89 (m, 5H), 1.91-1.96 (m, 4H), 2.31- 2.40 (m, 1H), 2.76 (brs, 1H), 2.87-2.95 (m, 5H), 4.27-4.36 (m, 1H), 4.38 (t, J = 7.57 Hz, 1H), 6.93 (t, J = 7.57 Hz, 1H), 6.96-7.00 (m, 1H), 7.06 (t, J = 7.11 Hz, 1H), 7.14-7.20 (m, 2H), 7.28-7.34 (m, 1H), 7.39 (d, J = 7.90 Hz, 1H), 7.46 (d, J = 8.33 Hz, 1H), 7.57 (s, 1H), 8.01 (brs, 3H), 9.17 (brs, 1H), 9.26 (brs, 1H). |
| 345 | 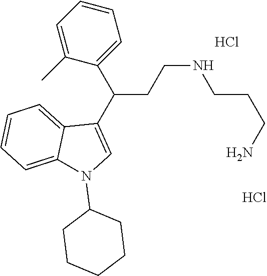 [SMILES: Cc1ccccc1C(CCNCCCN)c1cn(C2CCCCC2)c2ccccc12 ; Cl ; Cl] | ¹H NMR (400 MHz, DMSO-d₆): δ 1.22-1.31 (m, 1H), 1.44- 1.54 (m, 2H), 1.69-1.77 (m, 3H), 1.83 (d, J = 12.2 Hz, 2H), 1.92 (m, 4H), 2.24-2.31 (m, 1H), 2.40 (s, 3H), 2.85-2.95 (m, 6H), 4.29 (t, J = 11.59 Hz, 1H), 4.44 (t, J = 7.07 Hz, 1H), 6.89 (t, J = 7.38 Hz, 1H), 7.04-7.11 (m, 3H), 7.14 (d, J = 7.17 Hz, 2H), 7.19 (d, J = 7.37 Hz, 1H), 7.30 (d, J = 7.86 Hz, 1H), 7.44-7.46 (m, 2H), 7.99 (brs, 3H), 9.17 (brs, 2H). |
| 346 | 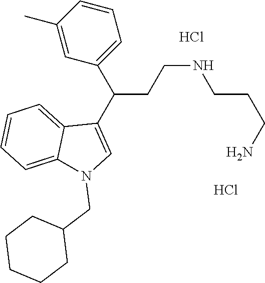 [SMILES: Cc1cccc(C(CCNCCCN)c2cn(CC3CCCCC3)c3ccccc23)c1 ; Cl ; Cl] | ¹H NMR (400 MHz, DMSO-d₆): δ 0.96-0.98 (m, 2H), 1.12- 1.24 (m, 3H), 1.26-1.29 (m, 1H), 1.49 (brs, 2H), 1.59-1.64 (m, 3H), 1.74 (brs, 2H), 1.93 (brs, 2H), 2.22 (s, 3H), 2.70- 2.74 (m, 2H), 2.87-3.09 (m, 7H), 3.58 (s, 1H), 3.97 (d, J = 5.06 Hz, 2H), 4.25 (t, J = 6.95 Hz, 1H), 6.89 (t, J = 7.32 Hz, 1H), 6.96 (d, J = 5.52 Hz, 1H), 7.0 (t, J = 7.71 Hz, 1H), 7.11-7.15 (m, 3H), 7.36-7.45 (m, 3H), 8.02 (brs, 3H), 9.23 (brs, 2H). |
| 347 | 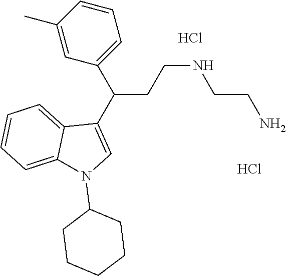 [SMILES: Cc1cccc(C(CCNCCN)c2cn(C3CCCCC3)c3ccccc23)c1 ; Cl ; Cl] | ¹H NMR (400 MHz, DMSO-d₆): δ 1.22-1.32 (m, 1H), 1.45- 1.51 (m, 2H), 1.54-1.86 (m, 5H), 1.95 (d, J = 7.45 Hz, 2H), 2.24 (s, 3H), 2.31 (s, 1H), 2.37-2.43 (m, 1H), 2.85-2.87 (m, 1H), 2.94 (brs, 1H), 3.14 (s, 4H), 4.25-4.32 (m, 2H), 6.90 (t, J = 7.44 Hz, 1H), 6.97 (d, J = 6.47 Hz, 1H), 7.07 (t, J = 7.89 Hz, 1H), 7.14-7.17 (m, 3H), 7.39 (d, J = 7.86 Hz, 1H), 7.45 (d, J = 8.02 Hz, 1H), 7.51 (s, 1H), 8.19 (brs, 3H), 9.36 (brs, 1H), 9.44 (brs, 1H). |
| 348 | 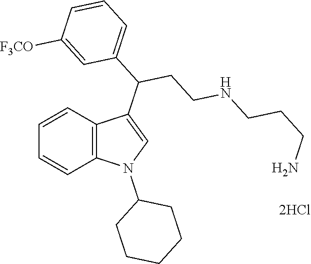 [SMILES: NCCCNCCC(c1cccc(OC(F)(F)F)c1)c1cn(C2CCCCC2)c2ccccc12] | ¹H NMR (400 MHz, DMSO-d₆): δ 1.22-1.32 (m, 1H), 1.45- 1.55 (m, 2H), 1.70-1.82 (m, 3H), 1.86 (d, J = 14.80 Hz, 2H), 1.86-1.96 (m, 4H), 2.35-2.37 (m, 1H), 2.52-2.57 (m, 1H), 2.78 (brs, 1H), 2.86-2.88 (m, 3H), 2.94 (t, J = 6.65 Hz, 1H), 4.27-4.33 (m, 1H), 4.43 (t, J = 7.75 Hz, 1H), 6.92 (t, J = 7.47 Hz, 1H), 7.07 (t, J = 7.78 Hz, 1H), 7.15 (d, J = 7.56 Hz, 1H), 7.33 (s, 1H), 7.36-7.43 (m, 3H), 7.47 (d, J = 8.34 Hz, 1H), 7.59 (s, 1H), 7.98 (brs, 3H), 9.11 (brs, 1H), 9.20 (brs, 1H). |
| 349 | 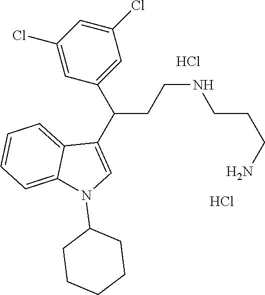 [SMILES: NCCCNCCC(c1cc(Cl)cc(Cl)c1)c1cn(C2CCCCC2)c2ccccc12 ; Cl ; Cl] | ¹H NMR (400 MHz, DMSO-d₆): δ 1.22-1.32 (m, 1H), 1.49- 1.52 (m, 2H), 1.70-1.84 (m, 6H), 1.93-1.95 (m, 4H), 2.34- 2.37 (m, 1H), 2.77 (brs, 1H), 2.87 (brs, 3H), 2.96 (brs, 2H), 4.27-4.33 (m, 1H), 4.42-4.43 (m, 1H), 6.94 (t, J = 6.26 Hz, 1H), 7.04 (t, J = 7.74 Hz, 1H), 7.40 (s, 4H), 7.48 (d, J = 8.32 Hz, 1H), 7.99 (brs, 3H), 9.14 (brs, 1H), 9.26 (brs, 1H). |
| 350 | 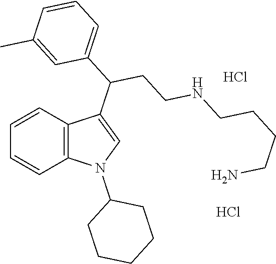 [SMILES: Cc1cccc(C(CCNCCCCN)c2cn(C3CCCCC3)c3ccccc23)c1 ; Cl ; Cl] | ¹H NMR (400 MHz, DMSO-d₆): δ 1.22-1.32 (m, 1H), 1.45- 1.58 (m, 6H), 1.70-1.79 (m, 3H), 1.84 (d, J = 12.33 Hz, 2H), 1.95 (d, J = 10.46 Hz, 2H), 2.24 (s, 3H), 2.28-2.33 (m, 1H), 2.76-2.78 (m, 3H), 2.87 (brs, 3H), 4.22 (t, J = 7.63 Hz, 1H), 4.26-4.32 (m, 1H), 6.90 (t, J = 7.28 Hz, 1H), 6.96 (d, J = 6.70 Hz, 1H), 7.05 (d, J = 7.61 Hz, 1H), 7.11-7.17 (m, 3H), 7.38 (d, J = 7.89 Hz, 1H), 7.45 (d, J = 8.23 Hz, 1H), 7.78 (s, 1H), 7.85 (brs, 3H), 8.86 (brs, 1H), 8.91 (brs, 1H). |
| 351 | 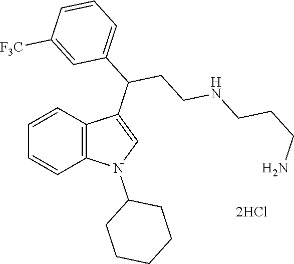 [SMILES: NCCCNCCC(c1cccc(C(F)(F)F)c1)c1cn(C2CCCCC2)c2ccccc12 ; "2HCl"] | ¹H NMR (400 MHz, DMSO-d₆): δ 9.06-9.15 (m, 2H), 7.95 (s [symbol] 3H), 7.70 (s, 1H), 7.65-7.62 (m, 2H), 7.53-7.51 (m, 2H), 7.47 [symbol] (d, J = 8.26 Hz, 1H), 7.38 (d, J = 7.88 Hz, 1H), 7.08 (t, J = 7.51 Hz, 1H), 6.92 (t, J = 7.44 Hz, 1H), 4.50 (t, J = 7.52 Hz, 1H), 4.31 (t, J = 11.33 Hz, 1H), 3.0- 2.92 (m, 2H), 2.90-2.85 (m, 3H), 2.82-2.73 (m, 1H), 2.60-2.55 (m, 1H), 2.40-2.35 (m, [symbol] 1H), 2.0-1.70 (m, 9H), 1.56-1.46 (m, 2H), 1.33-1.29 (m, 1H). |
| 352 | 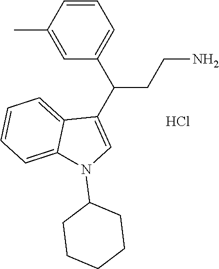 [SMILES: Cc1cccc(C(CCN)c2cn(C3CCCCC3)c3ccccc23)c1 ; Cl] | ¹H NMR (400 MHz, DMSO-d₆): δ 1.22-1.32 (m, 1H), 1.48- 1.51 (m, 2H), 1.74-1.80 (brs, 3H), 1.85 (d, J = 12.15 Hz, 2H), 1.95 (d, J = 11.70 Hz, 2H), 2.21 (s, 1H), 2.24 (s, 3H), 2.53-2.56 (m, 2H), 2.72-2.77 (m, 1H), 4.21 (t, J = 7.70 Hz, 1H), 4.29 (t, J = 10.90 Hz, 1H), 6.90 (t, J = 7.14 Hz, 1H), 6.96 (d, J = 6.81 Hz, 1H), 7.06 (t, J = 7.17 Hz, 1H), 7.10- 7.18 (m, 3H), 7.68 (brs, 2H). |
| 353 | 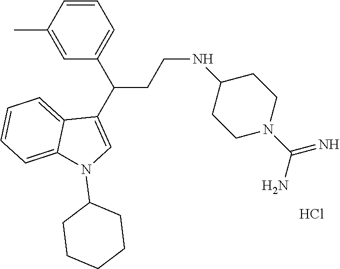 [SMILES: Cc1cccc(C(CCNC2CCN(C(=N)N)CC2)c2cn(C3CCCCC3)c3ccccc23)c1 ; Cl] | ¹H NMR (400 MHz, DMSO-d₆): δ 1.07 (t, J = 6.96 Hz, 1H), 1.22-132 (m, 1H), 1.45-1.58 (m, 4H), 1.70-1.86 (m, 5H), 1.94-2.03 (m, 4H), 2.24 (s, 3H), 2.31-2.35 (m, 1H), 2.80 (brs, 1H), 2.90 (brs, 1H), 2.98 (t, J = 12.69 Hz, 2H), 3.36 (brs, 1H), 3.91 (d, J = 12.87 Hz, 1H), 4.26-4.28 (m, 2H), 6.90 (t, J = 7.26 Hz, 1H), 6.94 (d, J = 17.87 Hz, 1H), 7.05 (t, J = 7.33 Hz, 1H), 7.13 (s, 3H), 7.39 (d, J = 7.76 Hz, 1H), 7.44 (d, J = 8.28 Hz, 1H), 7.52-7.55 (m, 5H), 9.41 (brs, 1H), 9.53 (brs, 1H). |
| 354 | 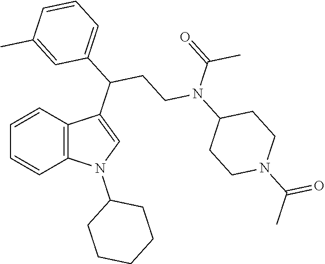 [SMILES: CC(=O)N1CCC(N(CCC(c2cccc(C)c2)c2cn(C3CCCCC3)c3ccccc23)C(C)=O)CC1] | ¹H NMR (400 MHz, DMSO-d₆): δ 0.81-0.85 (m, 1H), 1.22- 1.34 (m, 4H), 1.47-1.50 (m, 4H), 1.61-1.64 (m, 1H), 1.69- 1.77 (m, 3H), 1.86 (s, 4H), 1.93-1.94 (m, 5H), 2.04 (s, 2H), 2.21 (s, 1H), 2.23 (s, 3H), 2.39 (s, 1H), 2.94-3.02 (m, 2H), 3.06-3.17 (m, 1H), 3.76 (t, J = 11.71 Hz, 1H), 4.06-4.10 (m, 1H), 4.23-4.32 (m, 2H), 4.40 (d, J = 13.32 Hz, 1H), 6.85- 6.94 (m, 2H), 7.00-7.06 (m, 1H), 7.10-7.14 (m, 2H), 7.17 (d, J = 6.62 Hz, 1H), 7.39 (d, J = 7.86 Hz, 1H), 7.40-7.43 (m, 2H), 7.54 (d, J = 2.83 Hz, 1H). |
| 655 | 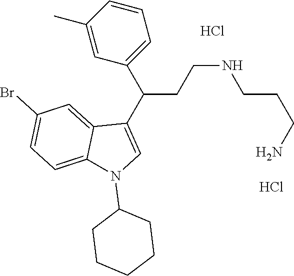 [SMILES: Cc1cccc(C(CCNCCCN)c2cn(C3CCCCC3)c3ccc(Br)cc23)c1 ; Cl ; Cl] | ¹H NMR (400 MHz, DMSO-d₆): δ 1.22-1.28 (m, 2H), 1.44- 1.54 (m, 2H), 1.69-1.94 (m, 5H), 1.91-1.94 (m, 4H), 2.24 (s, 3H), 2.74 (br s, 1H), 2.86-2.94 (m, 5H), 4.24-4.30 (m, 2H), 6.98 (d, J = 7.23 Hz, 1H), 7.12-7.16 (m, 4H), 7.47 (d, J = 8.79 Hz, 1H), 7.52 (s, 1H), 7.61 (s, 1H), 8.02 (brs, 3H), 9.18 (brs, 1H), 9.24 (brs, 1H). |
| 356 | 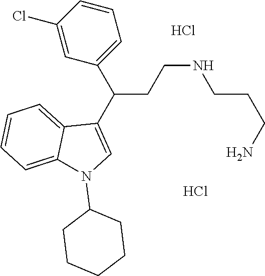 [SMILES: NCCCNCCC(c1cccc(Cl)c1)c1cn(C2CCCCC2)c2ccccc12 ; Cl ; Cl] | ¹H NMR (400 MHz, DMSO-d₆): δ 1.21-1.33 (m, 1H), 1.49- 1.52 (m, 2H), 1.70-1.90 (m, 6H), 1.92-1.94 (m, 4H), 2.34- 2.35 (m, 1H), 2.65-2.77 (m, 1H), 2.88-2.96 (m, 5H), 4.27- 4.33 (m, 1H), 4.37 (t, J = 7.55 Hz, 1H), 6.92 (t, J = 7.35 Hz, 1H), 7.07 (t, J = 7.41 Hz, 1H), 7.20-7.23 (m, 1H), 7.28- 7.32 (m, 2H), 7.38 (d, J = 7.42 Hz, 2H), 7.46 (d, J = 8.28 Hz, 1H), 7.58 (s, 1H), 7.97 (br s, 3H), 9.10 (br s, 1H), 9.19 (br s, 1H). |
| 357 | 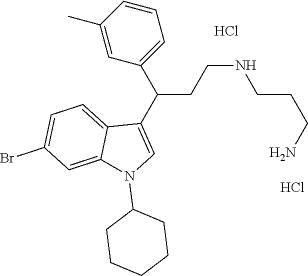 [SMILES: Cc1cccc(C(CCNCCCN)c2cn(C3CCCCC3)c3cc(Br)ccc23)c1 ; Cl ; Cl] | ¹H NMR (400 MHz, DMSO-d6) δ 9.25-9.07 (m, 2H), 8.25- 8.04 (m, 3H), 7.74 (s, 1H), 7.60 (s, 1H), 7.30 (d, J = 8.29 Hz, 1H), 7.16-7.04 (m, 3H), 7.03 (d, J = 8.27 Hz, 1H), 6.96 (d, J = [symbol] 6.7 Hz, 1H), 4.39-4.32(m,, 1H), 4.24 (t, J = 6.95 Hz, 1H), 2.9 [symbol] -2.85 (m, 5H), 2.84-2.74 (m, 1H), 2.38-2.30 (m, 1H), 2.23(s, 3H), 2.0-1.9 (m, 4H), 1.80-1.62 (m, 5H), 1.55-1.43 (m, 2H), 1.33-1.20 (m, 1H). |
| 358 | 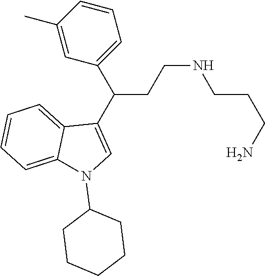 [SMILES: Cc1cccc(C(CCNCCCN)c2cn(C3CCCCC3)c3ccccc23)c1] | ¹H NMR (400 MHz, DMSO-d₆): δ 1.22-1.34 (m, 3H), 1.42- 1.52 (m, 4H), 1.70-1.86 (m, 4H), 1.81-1.83 (m, 2H), 1.91- 1.93 (m, 2H), 2.22 (brs, 1H), 2.31 (s, 4H), 3.08 (s, 1H), 4.16 (t, J = 7.39 Hz, 1H), 4.27 (t, J = 11.80 Hz, 1H), 6.86 (d, J = 7.07 Hz, 1H), 6.88-6.91 (m, 1H), 7.02 (t, J = 7.97 Hz, 1H), 7.09 (brs, 3H), 7.34-7.42 (m, 3H). |
| 359 | 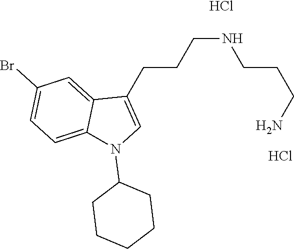 [SMILES: Cl ; NCCCNCCCc1cn(C2CCCCC2)c2ccc(Br)cc12 ; Cl] | ¹H NMR (400 MHz, DMSO-d₆): δ 1.22-1.28 (m, 2H), 1.43- 1.53 (m, 2H), 1.65-1.71 (m, 3H), 1.83 (d, J = 13.33 Hz, 2H), 1.90-1.97 (m, 6H), 2.73 (t, J = 7.48 Hz, 2H), 2.88-2.96 (m, 6H), 4.25-4.31 (m, 1H), 7.21 (dd, J_{1,2} = 1.80 Hz, J_{1,3} = 8.80 Hz, 1H), 7.48 (d, J = 8.86 Hz, 1H), 7.72 (d, J = 1.71 Hz, 1H), 7.93 (brs, 3H), 8.82 (brs, 1H), 8.84 (brs, 1H). |
| 360 | 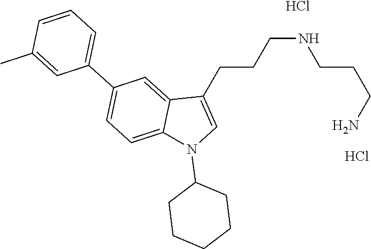 [SMILES: Cl ; Cc1cccc(-c2ccc3c(c2)c(CCCNCCCN)cn3C2CCCCC2)c1 ; Cl] | ¹H NMR (400 MHz, DMSO-d₆): δ 1.22-1.30 (m, 2H), 1.49- 1.55 (m, 2H), 1.71-1.78 (m, 3H), 1.85 (d, J = 13.29 Hz, 2H), 1.94-2.02 (m, 6H), 2.38 (s, 4H), 2.81 (t, J = 7.48 Hz, 2H), 2.89-2.95 (m, 7H), 4.30 (t, J = 11.52 Hz, 1H), 7.10 (d, J = 7.21 Hz, 1H), 7.30 (d, J = 7.70 Hz, 1H), 7.34 (s, 1H), 7.39 (d, J = 8.49 Hz, 1H), 7.45 (d, J = 7.62 Hz, 1H), 7.49 (s, 1H), 7.54 (d, J = 8.42 Hz, 1H), 7.73 (s, 1H), 7.97 (brs, 3H), 8.93 (brs, 2H). |
| 361 | 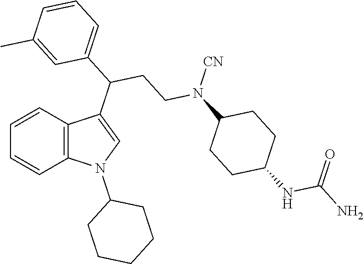 | ¹H NMR (400 MHz, DMSO-d₆): δ 1.00-1.10 (m, 2H), 1.22- 1.32 (m, 4H), 1.44-1.54 (m, 2H), 1.70-1.89 (m, 10H), 1.94 (d, J = 11.64 Hz, 2H), 2.24 (s, 4H), 2.37-2.38 (m, 1H), 2.71 (t, J = 11.52 Hz, 1H), 2.90-2.65 (m, 2H), 3.15-3.20 (m, 1H), 4.17 (t, J = 7.82 Hz, 1H), 4.28 (t, J = 11.46 Hz, 1H), 5.29 (s, 1H), 5.75 (d, (t, J = 4.52 Hz, 1H), 6.90 (t, J = 7.41 Hz, 1H), 6.93-6.95 (m, 1H), 7.05 (t, J = 7.75 Hz, 1H), 7.13 (d, J = 5.57 Hz, 3H), 7.41 (dd, J_{1,2} = 7.04 Hz, J_{1,3} = 14.93 Hz, 2H), 7.48 (s, 1H). |
| 362 | 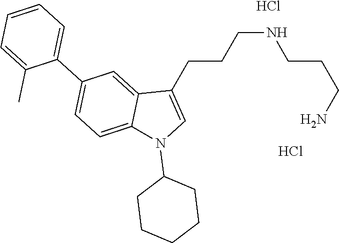 [SMILES: Cc1ccccc1-c1ccc2c(c1)c(CCCNCCCN)cn2C1CCCCC1 ; Cl ; Cl] | ¹H NMR (400 MHz, DMSO-d₆): δ 1.22-1.27 (m, 2H), 1.44- 1.54 (m, 2H), 1.73-1.95 (m, 11H), 2.25 (s, 4H), 2.76-2.89 (m, 9H), 4.28 (brs, 1H), 7.06-7.52 (m, 8H), 8.05 (s, 3H), 9.02 (brs, 2H). |
| 363 | 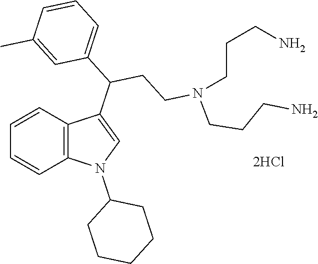 [SMILES: Cc1cccc(C(CCN(CCCN)CCCN)c2cn(C3CCCCC3)c3ccccc23)c1] | ¹H NMR (400 MHz, DMSO-d₆): δ 1.22-1.32 (m, 1H), 1.48- 1.51 (m, 2H), 1.70-1.87 (m, 5H), 1.94-1.96 (m, 6H), 2.24 (s, 3H), 2.63-2.65 (m, 1H), 2.88 (brs, 5H), 3.19 (m, 1H), 3.22 (brs, 5H), 4.18-4.20 (m, 1H), 4.21-4.30 (m, 1H), 6.91 (t, J = 7.48 Hz, 1H), 6.96 (d, J = 5.60 Hz, 1H), 7.07 (t, J = 7.55 Hz, 1H), 7.15-7.17 (m, 3H), 7.44 (dd, J = 6.80 Hz, 2H), 7.68 (s, 1H), 8.08 (brs, 6H), 11.19 (brs, 1H). |
| 364 | 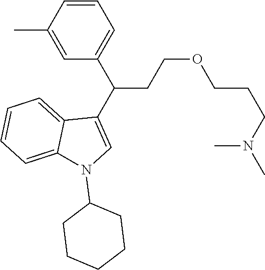 [SMILES: Cc1cccc(C(CCOCCCN(C)C)c2cn(C3CCCCC3)c3ccccc23)c1] | ¹H NMR (400 MHz, DMSO-d₆): δ 1.22-1.31 (m, 2H), 1.44- 1.53 (m, 2H), 1.59-1.62 (m, 2H), 1.73-1.77 (m, 3H), 1.84 (d, J = 12.82 Hz, 1H), 1.94 (d, J = 11.13 Hz, 1H), 2.17 (s, 7H), 2.28 (s, 3H), 2.31-2.33 (m, 4H), 3.16-3.25 (m, 4H), 4.20 (t, J = 7.41 Hz, 1H), 4.27 (t, , J = 11.55 Hz, 1H), 6.87 (t, J = 7.46 Hz, 1H), 6.91 (d, J = 6.48 Hz, 2H), 7.03 (t, J = 7.35 Hz, 1H), 7.10-7.13 (m, 3H), 7.34 (d, J = 7.79 Hz, 1H), 7.42 (s, 1H). |
| 365 | 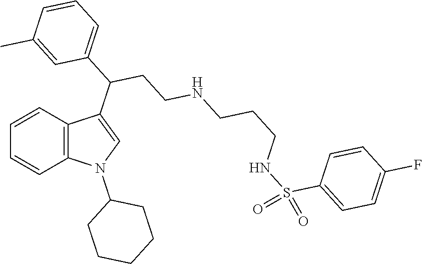 [SMILES: Cc1cccc(C(CCNCCCNS(=O)(=O)c2ccc(F)cc2)c2cn(C3CCCCC3)c3ccccc23)c1] | ¹H NMR (400 MHz, DMSO-d₆): δ 1.22-1.31 (m, 2H), 1.44- 1.50 (m, 4H), 1.69-1.85 (m, 3H), 1.84 (d, J = 12.31 Hz, 2H), 1.93 (d, J = 10.88 Hz, 2H), 2.06-2.08 (m, 2H), 2.22 (s, 4H), 2.41-2.43 (m, 4H), 2.76 (t, J = 6.67 Hz, 2H), 4.14 (t, J = 7.24 Hz, 1H), 4.26 (t, J = 11.18 Hz, 1H), 6.86 (d, J = 7.23 Hz, 1H), 6.90 (t, J = 5.05 Hz, 1H), 7.07 (t, J = 7.54 Hz, 1H), 7.01-7.10 (m, 4H), 7.33-7.42 (m, 5H), 7.81 (t, J = 5.32 Hz, 1H). |
| 366 | 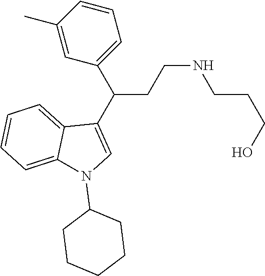 [SMILES: Cc1cccc(C(CCNCCCO)c2cn(C3CCCCC3)c3ccccc23)c1] | ¹H NMR (400 MHz, DMSO-d₆): δ 7.43-7.34 (m, 3H), 7.15- 7.09 (m, 3H), 7.03 (t, J = 7.41 Hz, 1H), 6.96-6.85 (m, 2H), 4.32-4.25 (m, 1H), 4.17 (t, J = 7.21 Hz, 1H), 2.40-2.32 (m, 6H), 2.22 (s, 4H), 2.10-2.05 (m, 1H), 1.98-1.91 (m, 2H), 1.89- [symbol] 1.80 (m, 2H), 1.78-1.70 (m, 3H), 1.63 (s, 4H), 1.54-1.45 (m, 4H), 1.36-1.20 (m, 3H). |
| 367 | 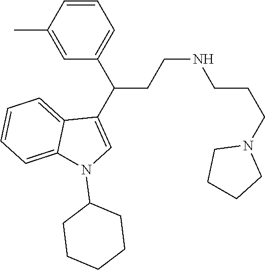 [SMILES: Cc1cccc(C(CCNCCCN2CCCC2)c2cn(C3CCCCC3)c3ccccc23)c1] | ¹H NMR (400 MHz, DMSO-d₆): δ 1.10-1.28 (m, 3H), 1.44- 1.51 (m, 4H), 1.62-1.68 (m, 6H), 1.81 (d, J = 11.59 Hz, 2H), 1.90 (d, J = 10.36 Hz, 2H), 2.09 (brs, 1H), 2.19 (s, 4H), 2.32-2.36 (m, 2H), 4.10-4.12 (m, 1H), 4.22 (brs, 1H), 6.84- 6.91 (m, 2H), 7.00-7.09 (m, 3H), 7.32-7.39 (m, 2H). |
| 368 | 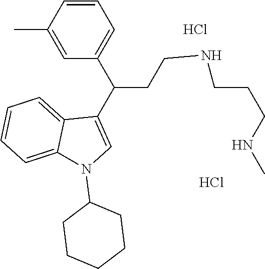 [SMILES: CNCCCNCCC(c1cccc(C)c1)c1cn(C2CCCCC2)c2ccccc12 ; Cl ; Cl] | ¹H NMR (400 MHz, DMSO-d₆): δ 1.22-1.32 (m, 2H), 1.46- 1.55 (m, 2H), 1.70-1.86 (m, 6H), 1.95 (d, J = 6.68 Hz, 5H), 2.24 (s, 4H), 2.31 (s, 1H), 2.72 (t, J = 3.38 Hz, 2H), 2.95 (d, J = 6.07 Hz, 6H), 4.22-4.32 (m, 2H), 6.90 (t, J = 7.33 Hz, 1H), 6.96 (d, J = 6.64 Hz, 1H), 7.05 (t, J = 7.79 Hz, 1H), 7.11-7.17 (m, 3H), 7.38 (d, J = 7.71 Hz, 1H),), 7.45 (d, J = 8.29 Hz, 1H), 7.49 (s, 1H), 8.78 (brs, 2H), 9.05 (brs, 2H). |
| 369 | 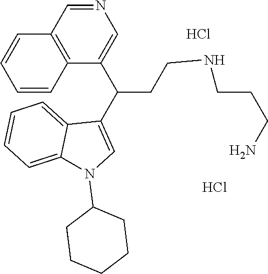 [SMILES: NCCCNCCC(c1cncc2ccccc12)c1cn(C2CCCCC2)c2ccccc12 ; Cl ; Cl] | ¹H NMR (400 MHz, DMSO-d₆): δ 1.22-1.32 (m, 2H), 1.46- 1.55 (m, 2H), 1.73-1.81 (m, 3H), 1.83 (d, J = 13.85 Hz, 3H), 1.93 (s, 6H), 2.58 (s, 1H), 2.66 (s, 1H), 2.88 (s, 2H), 2.96 (brs, 4H), 4.30 (t, J = 11.39 Hz, 1H), 2.52 (s, 1H), 6.90 (t, J = 7.41 Hz, 1H), 7.07 (t, J = 7.49 Hz, 1H), 7.38 (t, J = 8.05 Hz, 1H), 7.49 (t, J = 8.25 Hz, 1H), 7.65 (s, 1H), 7.84-8.00 (m, 5H), 8.33 (brs, 1H), 8.50 (s, 1H), 8.56 (brs, 1H), 9.15 (brs, 1H), 9.47 (brs, 1H). |
| 370 | 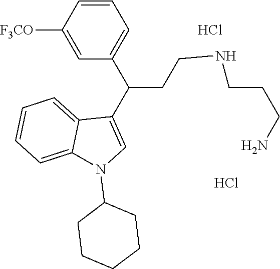 [SMILES: NCCCNCCC(c1cccc(OC(F)(F)F)c1)c1cn(C2CCCCC2)c2ccccc12 ; Cl ; Cl] | ¹H NMR (400 MHz, DMSO-d₆): δ 1.22-1.28 (m, 2H), 1.46- 1.56 (m, 2H), 1.69-1.78 (m, 3H), 1.85 (d, J = 18.50 Hz, 3H), 1.90-1.96 (m, 4H), 2.00-2.07 (m, 2H), 2.81-2.97 (m, 8H), 4.30-4.35 (m, 1H), 7.28 (d, J = 8.09 Hz, 1H), 7.38 (s, 1H), 7.45 (dd, J_{1,2} = 1.40 Hz, J_{1,3} = 8.68 Hz,), 7.54-7.59 (m, 2H), 7.62 (s, 1H), 7.74 (d, J = 7.83 Hz, 1H), 7.87 (d, J = 1.19 Hz, 1H), 8.01 (brs, 3H), 9.00 (brs, 2H). |
| 371 | 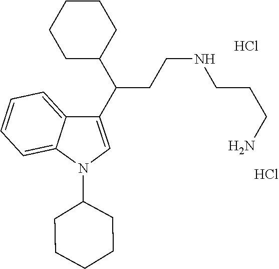 [SMILES: NCCCNCCC(c1cn(C2CCCCC2)c2ccccc12)C1CCCCC1 ; Cl ; Cl] | ¹H NMR (400 MHz, DMSO-d₆): δ 0.85-0.88 (m, 2H), 0.98- 1.44 (m, 4H), 1.47-1.65 (m, 7H), 1.68-1.74 (m, 4H), 1.81- 1.85 (m, 4H), 1.94 (s, 2H), 2.08 (s, 2H), 2.49-2.84 (m, 7H), 4.24-4.27 (m, 1H), 6.96 (t, J = 7.57 Hz, 1H), 7.07 (t, J = 7.38 Hz, 1H), 7.28 (s, 1H), 7.45 (d, J = 8.2 Hz, 1H), 7.51 (d, J = 7.91 Hz, 1H), 7.95 (br s, 3H), 8.92 (br s, 2H). |
| 372 | 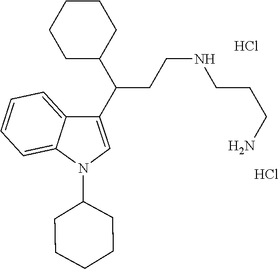 [SMILES: NCCCNCCC(c1cn(C2CCCCC2)c2ccccc12)C1CCCCC1 ; Cl ; Cl] | ¹H NMR (400 MHz, DMSO-d₆): δ 9.38-9.30 (m, 2H), 8.00- 7.70 (m, 5H), 7.71 (d, J = 8.61 Hz, 1H), 7.44-7.42 (m, 4H), 7.18 (brs, 1H), 4.55 (t, J = 7.40 Hz, 1H), 4.42-4.39 (m, 1H), 3.10-2.77 (m, 6H), 2.60-2.55 (m, 1H), 2.43-2.38 (m, 1H), 1.95-1.93 (m, 4H), 1.86-1.81 (m, 4H), 1.77-1.73 (m, 1H), 1.59-1.42 (m, 2H), 1.35-1.20 (m, 2H). |
| 373 | 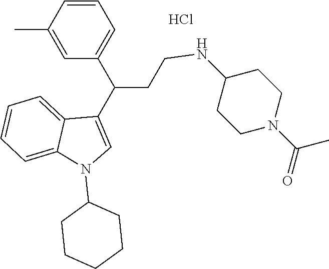 [SMILES: CC(=O)N1CCC(NCCC(c2cccc(C)c2)c2cn(C3CCCCC3)c3ccccc23)CC1 ; Cl] | ¹H NMR (400 MHz, DMSO-d₆): δ 1.08 (t, J = 7.02 Hz, 1H), 1.22-1.32 (m, 3H), 1.43-1.52 (m, 3H), 1.70-1.79 (m, 3H), 1.85 (d, J = 11.70 Hz, 2H), 1.90-1.97 (m, 7H), 2.80 (brs, 1H), 2.91-3.02 (m, 2H), 3.26 (brs, 2H), 3.31(s, 1H), 3.85 (d, J = 13.06 Hz, 1H), 4.22-4.32 (m, 2H), ), 4.38 (d, J = 12.34 Hz, 1H), 6.90 (t, J = 7.47 Hz, 1H), 6.96 (d, J = 6.53 Hz, 1H), 7.06 (t, J = 7.38 Hz, 1H), 7.14-7.17 (m, 3H), 7.39 (d- J = 7.81 Hz, 1H), 7.45 (d, J = 8.23 Hz, 1H), 7.50 (s, 1H), 8.97 (brs, 2H). |
| 374 | 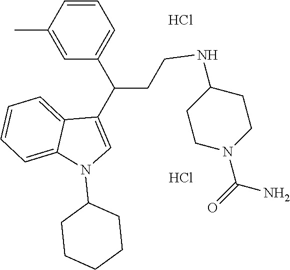 [SMILES: Cc1cccc(C(CCNC2CCN(C(N)=O)CC2)c2cn(C3CCCCC3)c3ccccc23)c1 ; Cl ; Cl] | ¹H NMR (400 MHz, DMSO-d₆): δ 1.22-1.32 (m, 3H), 1.45- 1.55 (m, 2H), 1.70-1.76 (m, 3H), 1.85 (d, J = 11.84 Hz, 4H), 1.90-1.97 (m, 2H), 2.24 (s, 4H), 2.59 (s, 1H), 2.80 (brs, 1H), 2.92 (brs, 1H), 3.21 (brs, 1H), 3.94 (d, J = 12.95 Hz, 2H), 4.22-4.32 (m, 2H), 6.91 (t, J = 7.40 Hz, 1H), 6.96 (d, J = 6.87 Hz, 1H), 7.06 (t, J = 7.77 Hz, 1H), 7.11-7.18 (m, 3H), 7.39 (d, , J = 7.84 Hz, 1H), 7.45 (d, J = 6.09 Hz, 1H), 8.68 (brs, 2H). |
| 375 | 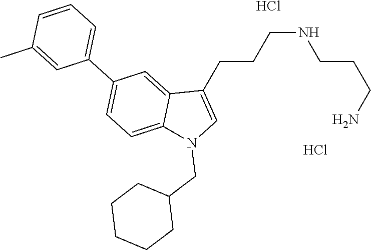 [SMILES: Cc1cccc(-c2ccc3c(c2)c(CCCNCCCN)cn3CC2CCCCC2)c1 ; Cl ; Cl] | ¹H NMR (400 MHz, DMSO-d₆): δ 0.96-1.1 (m, 2H), 1.12- 1.22 (m, 3H), 1.50 (t, J = 11.26 Hz, 2H), 1.59-1.64 (m, 3H), 1.70-1.77 (m, 1H), 1.94 (t, J = 6.82 Hz, 2H), 2.01 (t, J = 7.69 Hz, 1H), 2.37 (s, 3H), 2.79 (t, J = 7.33 Hz, 2H), 2.89- 2.95 (m, 6H), 3.96 (d, J = 6.94 Hz, 2H), 7.10 (d, J = 7.39 Hz, 1H), 7.14 (s, 1H), 7.31 (t, J = 7.55 Hz, 1H), 7.39 (d, J = 8.07 Hz, 1H), 7.44-7.49 (m, 3H), 7.77 (s, 1H), 8.03 (br s, 3H), 9.01 (br s, 2H). |
| 376 | 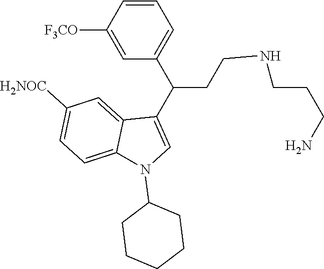 [SMILES: NCCCNCCC(c1cccc(OC(F)(F)F)c1)c1cn(C2CCCCC2)c2ccc(C(N)=O)cc12] | ¹H NMR (400 MHz, DMSO-d₆): δ 8.10 (brs, 1H), 7.80 (brs, 1H), 7.68-7.61 (m, 2H), 7.51 (d, J = 8.69 Hz, 1H), 7.40-7.34 (m, 3H), 7.13-7.07 (m, 2H), 4.34 (t, J = 11.72 Hz, 1H), 2.85- 2.81 (m, 2H), 2.74-2.70 (m, 2H), 2.29-2.25 (m, 2H), 2.00- 1.9 [symbol] (m, 2H), 1.89 (s, 1H), 1.87-1.72 (m, 7H), 1.54-1.45 (m, 2H), 1.32-1.22 (m, 4H). |
| 377 | 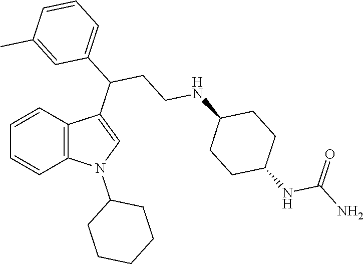 [SMILES: Cc1cccc(C(CCN[C@H]2CC[C@H](NC(N)=O)CC2)c2cn(C3CCCCC3)c3ccccc23)c1] | ¹H NMR (400 MHz, DMSO-d₆): δ 0.96-1.05 (m, 4H), 1.22- 1.32 (m, 2H), 1.44-1.54 (m, 2H), 1.70-1.76 (m, 7H), 1.85 (d, J = 13.03 Hz, 2H), 1.90-1.97 (d, J = 12.19 Hz, 2H), 2.06- 2.12 (m, 1H), 2.23 (s, 4H), 2.57 (s, 1H), 3.19 (brs, 1H), 4.17 (t, J = 7.73 Hz, 1H), 4.27 (t, J = 11.46 Hz, 1H), 5.75 (d, J = 7.91 Hz, 1H), 6.88 (t, J = 7.35 Hz, 1H), 6.92 (d, J = 6.36 Hz, 1H), 7.03 (t, J = 7.77 Hz, 1H), 7.10-7.14 (m, 3H), 7.35- 7.45 (d, 3H). |
| 378 | 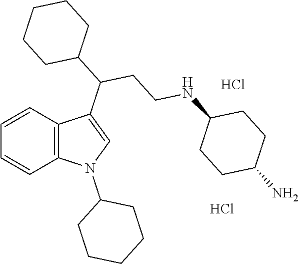 [SMILES: N[C@H]1CC[C@H](NCCC(c2cn(C3CCCCC3)c3ccccc23)C2CCCCC2)CC1 ; Cl ; Cl] | ¹H NMR (400 MHz, DMSO-d₆): δ 0.82-0.91 (m, 2H), 1.06 (t, 3H), 1.22-1.47 (m, 6H), 1.51-1.82 (m, 14H), 1.93 (s, 6H), 2.07-2.09 (m, 2H), 2.53-2.59 (m, 1H), 2.76 (t, J = 6.62 Hz, 2H), 2.88 (brs, 2H), 4.27 (t, J = 11.16 Hz, 1H), 6.96 (t, J = 7.32 Hz, 1H), 7.07 (t, J = 7.43 Hz, 1H), 7.30 (s, 1H), 7.45 (d, J = 8.25 Hz, 1H), 7.51 (d, J = 7.79 Hz, 1H) 8.01 (br s, 3H), 8.82 (br s, 1H), 8.99 (br s, 1H). |
| 379 | 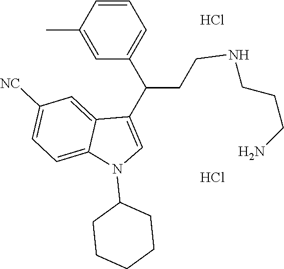 [SMILES: Cc1cccc(C(CCNCCCN)c2cn(C3CCCCC3)c3ccc(C#N)cc23)c1 ; Cl ; Cl] | ¹H NMR (400 MHz, DMSO-d₆): δ 1.22-1.35 (m, 2H), 1.48- 1.51 (m, 2H), 1.70 (d, J = 9.98 Hz, 2H), 1.83-1.96 (m, 9H), 2.25 (s, 4H), 2.87-2.96 (m, 7H), 4.35-4.40 (m, 2H), 6.99- 7.04 (m, 2H), 7.16 (s, 4H), 7.41 (d, J = 7.93 Hz, 1H), 7.69 (d, J = 7.77 Hz, 1H), 7.79 (s, 1H), 7.91 (s, 1H), 8.03 (brs, 3H), 9.22 (brs,, 2H). |
| 380 | 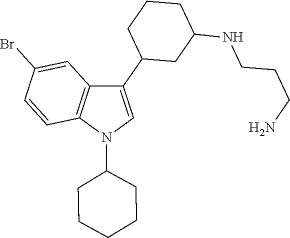 [SMILES: NCCCNC1CCCC(c2cn(C3CCCCC3)c3ccc(Br)cc23)C1] | ¹H NMR (400 MHz, DMSO-d₆): δ 0.95-1.04 (m, 1H), 1.14- 1.35 (m, 3H), 1.38-1.59 (m, 6H), 1.63-1.94 (m, 11H), 2.12 (d, J = 12.40 Hz, 1H), 2.56-2.66 (m, 5H), 2.77 (t, J = 11.74 Hz, 1H), 4.25 (t, J = 11.58 Hz, 1H), 7.17 (d, J = 8.73 Hz, 1H), 7.24 (d, J = 9.75 Hz, 1H), 7.45 (d, J = 8.75 Hz, 1H), 7.68 (s, 1H). |
| 381 | 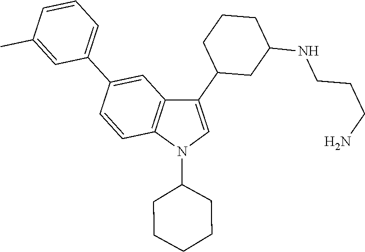 [SMILES: Cc1cccc(-c2ccc3c(c2)c(C2CCCC(NCCCN)C2)cn3C2CCCCC2)c1] | ¹H NMR (400 MHz, DMSO-d₆): δ 1.05-1.17 (m, 2H), 1.20- 1.35 (m, 4H), 1.31-1.50 (m, 4H), 1.53-1.58 (m, 2H), 1.60- 1.72 (m, 4H), 1.80 (d, J = 11.45 Hz, 3H), 1.89 (d, J = 11.68 Hz, 4H), 2.16 (d, J = 11.96 Hz, 1H), 2.33 (s, 3H), 2.64 (d, J = 6.58 Hz, 5H), 2.80-8.86 (m, 1H), 4.23 (t, J = 11.13 Hz, 1H), 7.08 (d, J = 7.44 Hz, 1H), 7.17 (s, 1H), 7.27-7.41 (m, 4H), 7.48 (d, J = 8.60 Hz, 1H), 7.70 (s, 1H). |
| 382 | 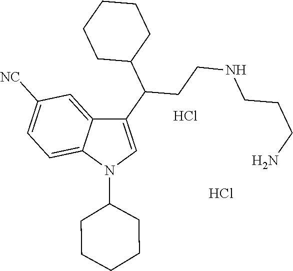 [SMILES: N#Cc1ccc2c(c1)c(C(CCNCCCN)C1CCCCC1)cn2C1CCCCC1 ; Cl ; Cl] | ¹H NMR (400 MHz, DMSO-d₆): δ 0.88-0.88 (m, 2H), 0.98- 1.27 (m, 5H), 1.47-1.62 (m, 7H), 1.65-1.92 (m, 11H), 2.07- 2.10 (m, 2H), 2.70 (brs, 1H), 2.84 (brs, 5H), 4.40 (t, J = 11.65 Hz, 1H), 7.43 (t, J = 8.74 Hz, 1H), 7.54 (s, 1H), 7.70 (d, J = 8.63 Hz, 1H), 7.92 (brs, 3H), 8.09 (s, 1H), 8.85 (brs, 2H). |
| 383 | 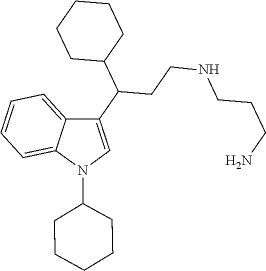 [SMILES: NCCCNCCC(c1cn(C2CCCCC2)c2ccccc12)C1CCCCC1] | ¹H NMR (400 MHz, DMSO-d₆): δ 0.88-0.92 (m, 2H), 0.96- 1.16 (m, 3H), 1.21-1.30 (m, 3H), 1.32 (s, 1H), 1.39-1.60 (m, 8H), 1.63-1.71 (m, 4H),), 1.75-1.84 (m, 4H), 1.88 (d, J = 13.68 Hz, 2H), 2.28-2.31 (m, 2H), 2.42 (d, J = 5.59 Hz, 2H), 2.60 (brs, 2H), 2.70-2.71 (m, 1H), 4.25 (t, J = 11.71 Hz, 1H), 6.93 (t, J = 7.25 Hz, 1H), 7.05 (t, J = 7.44 Hz, 1H), 7.15 (s, 1H), 7.54 (s, 1H), 7.42 (d, J = 8.25 Hz, 1H), 7.48 (d, J = 7.86 Hz, 1H). |
| 384 | 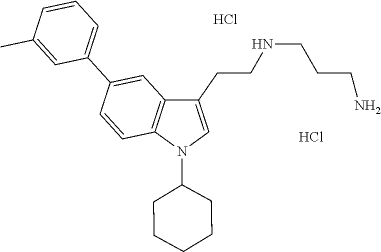 [SMILES: Cc1cccc(-c2ccc3c(c2)c(CCNCCCN)cn3C2CCCCC2)c1 ; Cl ; Cl] | ¹H NMR (400 MHz, DMSO-d6) δ 9.10 (brs, 1H), 9.05-8.98 (m, 3H), 7.86 (s, 1H), 7.57 (d, J = 8.75 Hz, 1H), 7.51 (s, 1H), 7.47 (d, J = 7.82 Hz, 1H), 7.43-7.41 (m, 2H), 7.31 (t, J = 7.57 [symbol] Hz, 1H), 7.10 (d, J = 7.25 Hz, 1H), 4.31 (t, J = 11.54 Hz, 1H) [symbol] 3.14 (s, 4H), 3.08-3.01 (m, 2H), 2.95- 2.86 (m, 2H), 2.37 (s, 3H), 1.96 (d, J = 8.87 Hz, 4H), 1.85 (d, J = 13.01 Hz, 2H), 1.80-1.66 (m, 3H), 1.50 (q, J = 25.47, 12.29 Hz, 2H), 1.25-1.20 (m, 2H). |
| 385 | 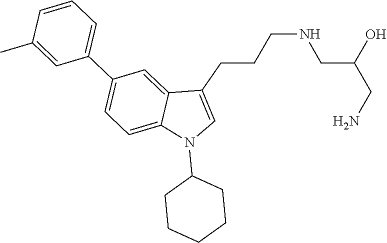 [SMILES: Cc1cccc(-c2ccc3c(c2)c(CCCNCC(O)CN)cn3C2CCCCC2)c1] | ¹H NMR (400 MHz, DMSO-d₆): δ 1.22-1.30 (m, 2H), 1.45- 1.55 (m, 2H), 1.70-1.86 (m, 7H), 1.95 (d, J = 11.59 Hz, 2H), 2.37 (s, 3H), 2.58-2.66 (m, 4H), 2.72-2.82 (m, 3H), 3.66 (brs, 1H), 4.29 (t, J = 11.84 Hz, 1H), 7.09 (d, J = 7.45 Hz, 1H), 7.26 (s, 1H), 7.31 (t, J = 7.46 Hz, 1H), 7.38 (t, J = 8.11 Hz, 1H), 7.43 (d, J = 7.76 Hz, 1H), 7.46 (s, 1H), 7.52 (d, J = 8.52 Hz, 1H), 7.72 (s, 1H). |
| 386 | 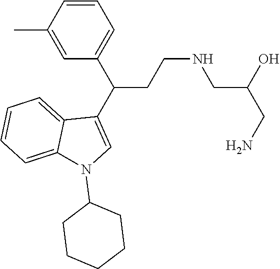 [SMILES: Cc1cccc(C(CCNCC(O)CN)c2cn(C3CCCCC3)c3ccccc23)c1] | ¹H NMR (400 MHz, DMSO-d₆): δ 1.22-1.34 (m, 4H), 1.45- 1.55 (m, 2H), 1.70-1.80 (m, 3H), 1.84 (d, J = 12.87 Hz, 2H), 1.95 (d, J = 10.64 Hz, 2H), 2.17-2.18 (m, 1H), 2.23 (s, 3H), 2.31-2.32 (m, 1H), 2.53-2.56 (m, 1H), 2.62-2.71 (m, 4H), 2.87 (d, J = 11.19 Hz. 1H), 3.76 (brs, 1H), 4.18 (t, J = 7.73 Hz, 1H), 4.25-4.31 (m, 1H), 6.80 (brs, 1H), 6.87-6.94 (m, 3H), 7.04 (t, J = 7.76 Hz, 1H), 7.09-7.15 (m, 3H), 7.37 (d, J = 7.88 Hz, 1H), 7.40 (s, 1H), 7.43 (d, J = 8.29 Hz, 1H), 7.46 (s, 1H), 7.52 (d, J = 8.52 Hz, 1H), 7.72 (s, 1H). |
| 387 | 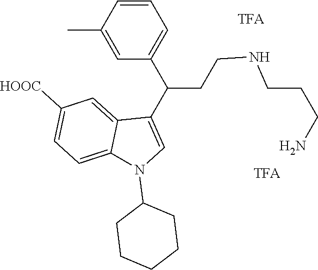 [SMILES: Cc1cccc(C(CCNCCCN)c2cn(C3CCCCC3)c3ccc(C(=O)O)cc23)c1] | ¹H NMR (400 MHz, DMSO-d₆): δ 1.22-1.32 (m, 2H), 1.46- 1.55 (m, 2H), 1.70-1.85 (m, 7H), 1.95 (brs, 2H), 2.24 (s, 3H), 2.28-2.29 (m, 1H), 2.40-2.42 (m, 1H), 2.76-2.85 (m, 3H), 2.89-2.98 (m, 1H), 4.28-4.40 (m, 2H), 6.98 (d, J = 7.27 Hz, 1H), 7.02-7.12 (m, 2H), 7.17 (t, J = 7.51 Hz, 1H), 7.54-7.60 (m, 2H), 7.68 (d, J = 8.81 Hz, 1H), 7.87 (brs, 2H), 8.07 (s, 1H), 12.41 (brs, 2H). |
| 388 | 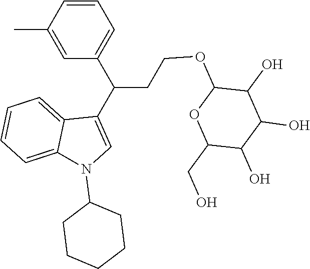 [SMILES: Cc1cccc(C(CCOC2OC(CO)C(O)C(O)C2O)c2cn(C3CCCCC3)c3ccccc23)c1] | ¹H NMR (400 MHz, DMSO-d₆): δ 1.20-1.32 (m, 3H), 1.43- 1.54 (m, 2H), 1.68-1.85 (m, 5H), 1.95 (d, J = 13.28 Hz, 2H), 2.16-2.20 (m, 1H), 2.22 (s, 3H), 2.32-2.38 (m, 1H), 3.20- 3.26 (m, 1H), 3.35-3.42 (m, 2H), 3.43-3.62 (m, 4H), 4.16- 4.-4.19 (m, 1H), 4.21-4.34 (m, 2H), 4.49 (d, J = 5.04 Hz, 1H), 4.53 (d, J = 5.90 Hz, 1H), 4.61-4.66 (m, 1H), 4.69 (d, J = 5.36 Hz, 1H), 6.85-6.93 (m, 2H), 7.03 (t, J = 8.00 Hz, 1H), 7.09-7.13 (m, 3H), 7.34 (d, J = 7.92 Hz, 1H), 7.41- 7.44 (m, 2H). |
| 389 | 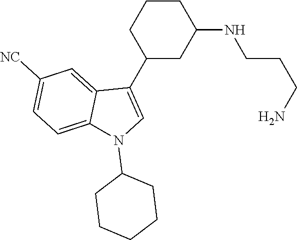 [SMILES: N#Cc1ccc2c(c1)c(C1CCCC(NCCCN)C1)cn2C1CCCCC1] | ¹H NMR (400 MHz, DMSO-d₆): δ 0.96-1.02 (m, 1H), 1.14- 1.34 (m, 4H), 1.39-1.55 (m, 6H), 1.56-1.94 (m, 11H), 2.13 (d, J = 11.51 Hz, 1H), 2.57-2.60 (m, 4H), 2.81-2.86 (m, 2H), 3.16-3.23 (m, 1H), 4.36 (t, J = 11.44 Hz, 1H), 7.42 (d, J = 11.48 Hz, 2H), 7.67 (d, J = 8.64 Hz, 1H), 8.09 (d, J = 7.18 Hz, 1H). |
| 390 | 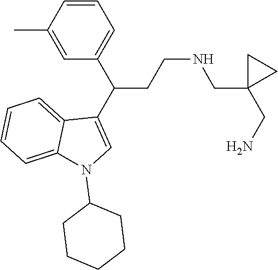 [SMILES: Cc1cccc(C(CCNCC2(CN)CC2)c2cn(C3CCCCC3)c3ccccc23)c1] | ¹H NMR (400 MHz, DMSO-d₆): δ 1.21-1.32 (m, 3H), 1.44- 1.53 (m, 2H), 1.69-1.79 (m, 3H), 1.84 (d, J = 13.17 Hz, 2H), 1.93 (d, J = 11.39 Hz, 2H), 2.06-2.10 (m, 1H), 2.19-2.22 (m, 1H), 2.24 (s, 3H), 2.40 (brs, 3H), 4.16 (brs, 1H), 4.24-4.30 (m, 1H), 6.87 (t, J = 7.42 Hz, 1H), 6.91(d, J = 5.12 Hz, 1H), 7.02 (t, J = 7.35 Hz, 1H), 7.09-7.12 (m, 3H), 7.35 (d, J = 7.87 Hz, 1H), 7.41 (d, J = 8.52 Hz, 1H). |
| 391 | 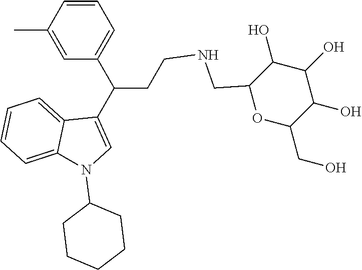 [SMILES: Cc1cccc(C(CCNCC2OC(CO)C(O)C(O)C2O)c2cn(C3CCCCC3)c3ccccc23)c1] | ¹H NMR (400 MHz, DMSO-d₆): δ 1.21-1.34 (m, 7H), 1.48- 1.51 (m, 2H), 1.70-1.76 (m, 3H), 1.84 (d, J = 13.22 Hz, 2H), 1.95 (d, J = 9.62 Hz, 2H), 2.24 (s, 4H), 2.40 (brs, 1H), 2.71 (brs, 1H), 2.93 (brs, 2H), 3.467-3.56 (m, 5H), 3.69 (brs, 1H), 3.83 (brs, 1H), 4.18 (t, J = 7.94 Hz, 1H), 4.26-4.31 (m, 1H), 4.45 (brs, 1H), 4.88 (brs, 1H), 6.88 (t, J = 7.60 Hz, 1H), 6.95(d, J = 6.73 Hz, 1H), 7.05 (t, J = 7.15 Hz, 1H), 7.09- 7.16 (m, 3H), 7.35 (d, J = 8.09 Hz, 1H), 7.42-7.45 (m, 1H). |
| 392 | 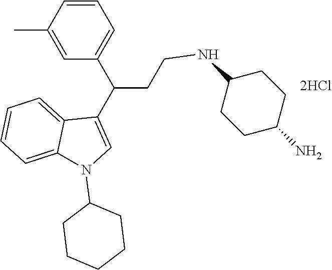 | ¹H NMR (400 MHz, DMSO-d₆): δ 1.24-1.40 (m, 7H), 1.45- 1.55 (m, 2H), 1.70-1.79 (m, 3H), 1.84 (d, J = 13.11 Hz, 2H), 1.94-2.03 (m, 7H), 2.24 (s, 3H), 2.25-2.29 (m, 1H), 2.78 (brs, 1H), 2.91-2.96 (m, 3H), 4.23 (t, J = 7.58 Hz, 1H), 4.29-4.32 (m, 1H), 6.90 (t, J = 7.39 Hz, 1H), 6.96 (d, J = 6.64 Hz, 1H), 7.05 (t, J = 7.76 Hz, 1H), 7.12-7.17 (m, 3H), 7.39 (d, J = 7.84 Hz, 1H), 7.45 (d, J = 8.24 Hz, 1H), 7.50 (s, 1H), 7.92-8.02 (m, 3H), 8.94 (brs, 1H), 9.04 (brs, 1H). |
| 393 | 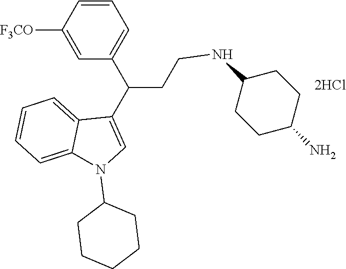 | ¹H NMR (400 MHz, DMSO-d₆): δ 1.22-1.54 (m, 9H), 1.70- 1.86 (m, 5H), 1.93-2.04 (m, 6H), 2.32-2.36 (m, 1H), 2.54- 2.56 (m, 1H), 2.72-3.06 (m, 4H), 4.27-4.33 (m, 1H), 4.45 (t, J = 7.55 Hz, 1H), 6.91 (t, J = 7.33 Hz, 1H), 7.06 (t, J = 7.47 Hz, 1H), 7.14 (d, J = 6.54 Hz, 1H), 7.34-7.41 (m, 4H), 7.46 (d, J = 8.27 Hz, 1H), 7.64 (s, 1H), 8.11 (s, 3H), 9.21 (brs, 1H), 9.336 (brs, 1H). |
| 394 | 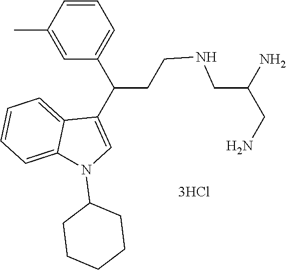 [SMILES: Cc1cccc(C(CCNCC(N)CN)c2cn(C3CCCCC3)c3ccccc23)c1] | ¹H NMR (400 MHz, DMSO-d₆): δ 1.22-1.34 (m, 2H), 1.45- 1.54 (m, 2H), 1.69-1.86 (m, 6H), 1.96 (d, J = 10.84 Hz, 2H), 2.24 (s, 3H), 2.35-2.38 (m, 1H), 2.84-2.88 (m, 1H), 2.35- 2.38 (m, 1H), 2.96 (brs, 1H), 3.12-3.17 (m, 4H), 3.80 (brs, 1H), 4.25-4.32 (m, 1H), 6.90 (t, J = 7.36 Hz, 1H), 6.97(d, J = 6.45 Hz, 1H), 7.06 (t, J = 7.38 Hz, 1H), 7.12-7.18 (m, 1H), 7.39 (d, J = 7.90 Hz, 1H), 7.45 (d, J = 8.33 Hz, 1H), 7.52 (d, J = 2.60 Hz, 1H), 8.31-8.75 (brs, 6H), 9.50 (brs, 2H). |
| 395 | 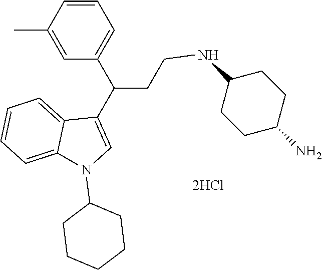 | ¹H NMR (400 MHz, DMSO-d₆): δ 1.22-1.31 (m, 1H), 1.36- 1.43 (m, 2H), 1.48-1.66 (m, 6H), 1.68-2.00 (m, 14H), 2.14 (brs, 4H), 2.14 (s, 4H), 2.93 (brs, 2H), 3.14 (brs, 1H), 3.55 (brs, 1H), 4.30 (t, J = 11.65 Hz, 1H), 7.10 (d, J = 7.30 Hz, 1H), 7.24 (s, 1H), 7.29-7.33(m, 2H), 7.39 (d, J = 8.36 Hz, 1H), 7.44 (d, J = 7.92 Hz, 1H), 7.49 (s, 1H), 7.54 (t, J = 8.61 Hz, 1H), 7.81 (d, J = 12.40 Hz, 1H), 8.08 (brs, 3H),), 8.78 (brs, 1H), 8.95 (brs, 1H). |
| 396 | 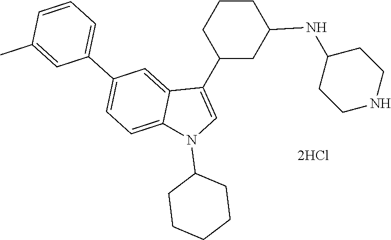 [SMILES: Cc1cccc(-c2ccc3c(c2)c(C2CCCC(NC4CCNCC4)C2)cn3C2CCCCC2)c1] | ¹H NMR (400 MHz, DMSO-d₆): δ 1.22-1.32 (m, 2H), 1.45- 1.60 (m, 5H), 1.63-1.86 (m, 8H), 1.94 (d, J = 10.99 Hz, 4H), 2.14 (brs, 3H), 2.38 (s, 3H), 2.88-2.98 (m, 3H), 3.30 (s, 2H), 3.50 (brs, 1H), 4.30 (t, J = 11.58 Hz, 1H), 7.10 (d, J = 7.47 Hz, 1H), 7.24 (s, 1H), 7.38 (t, J = 8.44 Hz, 1H), 7.44 (d, J = 7.65 Hz, 1H), 7.49 (s, 1H), 7.53-7.55 (m, 1H), 7.79 (s, 1H), 8.87 (brs, 3H). |
| 397 | 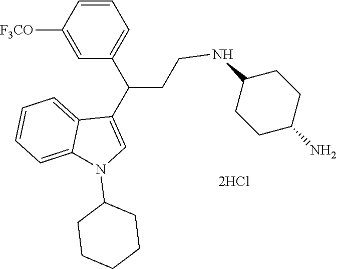 | ¹H NMR (400 MHz, DMSO-d₆): δ 1.21-1.52 (m, 12H), 1.56-1.78 (m, 4H), 1.84 (d, J = 13.00 Hz, 2H), 1.93-1.99 (m, 6H), 2.12 (brs, 3H), 2.36-2.39 (m, 1H), 2.95-3.0 (m, 2H), 3.18-3.19 (m, 1H), 3.45-3.53 (m, 1H), 4.33 (t, J = 11.57 Hz, 1H), 7.28 (s, 1H), 7.30 (s, 1H), 7.44 (d, J = 8.65 Hz, 1H), 7.55-7.60 (m, 3H), 7.73 (d, J = 7.63 Hz, 1H), 7.86 (s, 1H), 7.97 (brs, 3H), 8.69 (brs, 2H). |
| 398 | 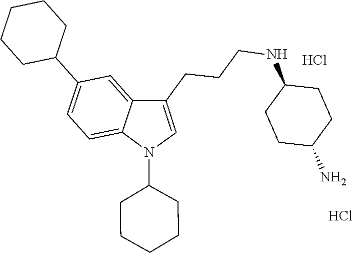 [SMILES: N[C@H]1CC[C@H](NCCCc2cn(C3CCCCC3)c3ccc(C4CCCCC4)cc23)CC1 ; Cl ; Cl] | ¹H NMR (400 MHz, DMSO-d₆): δ 1.21-1.32 (m, 3H), 1.35- 1.52 (m, 11H), 1.63-1.72 (m, 4H), 1.78-1.89 (m, 6H), 1.93- 2.00 (m, 6H), 2.07 (d, J = 10.01 Hz, 2H), 2.74 (t, J = 7.28 Hz, 2H), 2.94 (brs, 4H), 4.21 (t, J = 12.00 Hz, 1H), 6.98 (d, J = 8.19 Hz, 1H), 7.22 (s, 1H), 7.36 (t, J = 8.58 Hz, 1H), 7.98 (brs, 3H), 8.77 (brs, 2H). |
| 399 | 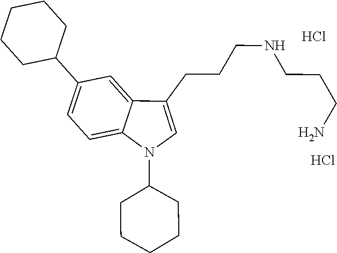 [SMILES: NCCCNCCCc1cn(C2CCCCC2)c2ccc(C3CCCCC3)cc12 ; Cl ; Cl] | ¹H NMR (400 MHz, DMSO-d₆): δ 1.22-1.32 (m, 3H), 1.35- 1.50 (m, 7H), 1.64-1.72 (m, 5H), 1.78-1.90 (m, 7H), 1.92- 1.99 (m, 7H), 2.72 (t, J = 7.07 Hz, 2H), 2.86-2.90 (m, 4H), 2.96 (t, J = 6.01 Hz, 2H), 4.21 (t, J = 11.86 Hz, 1H), 6.98 (d, J = 8.28 Hz, 1H), 7.23 (s, 1H), 7.31 (s, 1H), 7.35 (d, J = 8.61 Hz, 1H), 8.09 (brs, 3H), 8.93 (brs, 2H). |
| 400 | 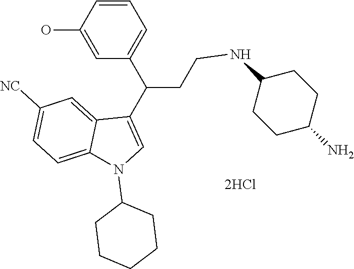 | ¹H NMR (400 MHz, DMSO-d₆): δ 1.23-1.47 (m, 6H), 1.50- 1.54 (m, 2H), 1.69-1.72 (m, 3H), 1.81-1.86 (m, 3H), 1.90- 1.97 (m, 7H), 2.36-2.39 (m, 1H), 2.74-2.78 (m, 1H), 2.87- 2.97 (m, 4H), 3.69 (s, 3H), 4.30 (t, J = 7.64 Hz, 1H), 4.35- 4.41 (m, 1H), 6.98 (d, J = 5.78 Hz, 1H), 6.90-6.94 (m, 2H), 7.21 (t, J = 7.80 Hz, 1H), 7.42 (d, J = 8.64 Hz, 1H), 7.67(s, 1H), 7.69(s, 1H), 7.93 (s, 1H). |
| 401 | 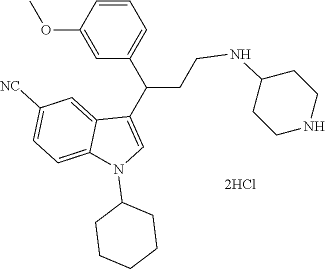 [SMILES: COc1cccc(C(CCNC2CCNCC2)c2cn(C3CCCCC3)c3ccc(C#N)cc23)c1] | ¹H NMR (400 MHz, DMSO-d₆): δ 1.20-1.30 (m, 2H), 1.45- 1.54 (m, 2H), 1.68-1.77 (m, 5H), 1.81-1.85 (m, 3H), 1.91 (d, J = 10.91 Hz, 2H), 2.06-2.09 (m, 2H), 2.74-2.95 (m, 5H), 3.28-3.34 (m, 3H), 3.69 (s, 3H), 4.32 (t, J = 7.51 Hz, 1H), 4.35-4.41 (m, 1H), 6.75 (d, J = 6.41 Hz, 1H), 6.90-6.94 (m, 2H), 7.21 (t, J = 7.88 Hz, 1H), 7.42 (d, J = 8.37 Hz, 1H), 7.67 (s, 1H), 7.69 (s, 1H), 7.93 (s, 1H). |
| 402 | 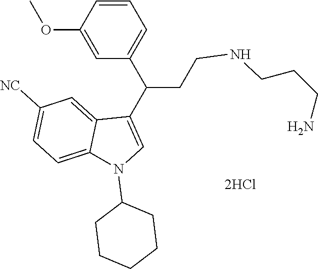 [SMILES: COc1cccc(C(CCNCCCN)c2cn(C3CCCCC3)c3ccc(C#N)cc23)c1] | ¹H NMR (400 MHz, DMSO-d₆): δ 1.21-1.32 (m, 2H), 1.48- 1.55 (m, 2H), 1.69-1.72 (m, 1H), 1.75-1.85 (m, 4H), 1.88- 1.94 (m, 4H), 2.35-2.39 (m, 1H), 2.74 (brs, 2H), 2.87(brs, 3H), 2.95 (brs, 2H), 3.71 (s, 3H), 4.35-4.44 (m, 2H), 6.76 (d, J = 9.43 Hz, 1H), 6.93 (s, 1H), 6.95 (s, 1H), 7.21 (t, J = 8.35 Hz, 1H), 7.43 (d, J = 8.57 Hz, 1H), 7.72 (d, J = 8.63 Hz, 1H), 7.76 (s, 1H), 7.94 (brs, 3H), 9.05 (brs, 2H). |
| 403 | 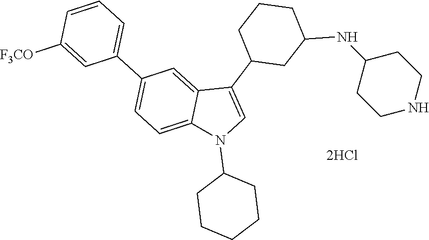 [SMILES: FC(F)(F)Oc1cccc(-c2ccc3c(c2)c(C2CCCC(NC4CCNCC4)C2)cn3C2CCCCC2)c1] | ¹H NMR (400 MHz, DMSO-d₆): δ 1.19-1.38 (m, 5H), 1.41- 1.54 (m, 5H), 1.70-1.75 (m, 6H), 1.83 (t, J = 12.26 Hz, 2H), 1.90-1.98 (m, 4H), 2.09-2.20 (m, 3H), 2.38 (s, 1H), 2.90 (t, J = 13.07 Hz,, 2H), 2.98-3.01 (m, 1H), 3.35 (m, 2H), 3.49- 3.52 (m, 1H), 4.30 (t, J = 11.70 Hz, 1H), 7.27-7.32 (m, 2H), 7.42 (d, J = 8.59 Hz, 1H), 7.54-7.58 (m, 3H), 7.71 (d, J = 7.78 Hz, 1H), 7.83-7.85 (m, 1H). |
| 404 | 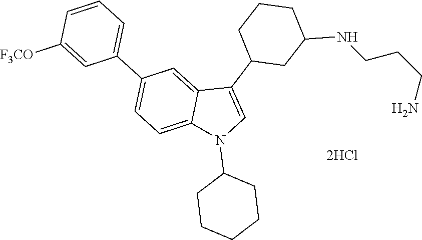 [SMILES: NCCCNC1CCCC(c2cn(C3CCCCC3)c3ccc(-c4cccc(OC(F)(F)F)c4)cc23)C1] | ¹H NMR (400 MHz, DMSO-d₆): δ 9.05-9.02 (m, 2H), 8.80- 8.74 (m, 2H), 7.89-7.88 (m, 1H), 7.74 (d, J = 7.88 Hz, 1H), 7.61-7.45 (m, 3H), 7.44 (d, J = 8.36 Hz, 1H), 7.30-7.28 (m, 2H), 4.33 (t, J = 11.56 Hz, 1H), 3.59-3.52 (m, 2H), 2.42- 2.38 [symbol] (m, 1H), 2.25-2.10 (m, 3H), 2.0-1.90 (m, 4H), 1.89-1.80 (m, 5H), 1.75-1.62(m, 3H), 1.60-1.40 (m, 6H), 1.32-1.30 (m, 1H) [symbol] |
| 405 | 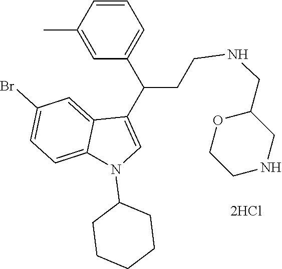 [SMILES: Cc1cccc(C(CCNCC2CNCCO2)c2cn(C3CCCCC3)c3ccc(Br)cc23)c1] | ¹H NMR (400 MHz, DMSO-d₆): δ 1.22-1.28 (m, 2H), 1.47- 1.54 (m, 2H), 1.50-1.85 (m, 5H), 1.92 (brs, 2H), 2.24 (s, 3H), 2.79 (brs, 2H), 2.93 (brs, 3H), 3.15-3.24 (m, 4H), 3.77 (t, J = 11.59 Hz, 2H), 3.96 (d, J = 11.81 Hz, 2H), 4.22-4.30 (m, 2H), 6.98 (d, J = 6.49 Hz, 1H), 7.11 (s, 2H), 7.16 (d, J = 7.50 Hz, 2H), 7.47 (d, J = 8.61 Hz, 1H), 7.52 (s, 1H), 7.57 (s, 1H), 8.99 (brs, 1H), 9.44 (brs, 1H), 9.57 (brs, 1H), (brs, 1H), 9.72 (brs, 1H). |
| 406 | 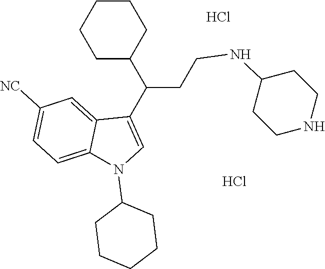 [SMILES: N#Cc1ccc2c(c1)c(C(CCNC1CCNCC1)C1CCCCC1)cn2C1CCCCC1 ; Cl ; Cl] | ¹H NMR (400 MHz, DMSO-d₆): δ 0.83-0.97 (m, 2H), 1.00- 1.03 (m, 1H), 1.08-1.32 (m, 4H), 1.45-1.60 (m, 6H), 1.65- 1.92 (m, 12H), 1.96-2.14 (m, 4H), 2.71-2.88 (m, 4H), 3.20 (brs, 1H), 3.28 (brs, 1H), 4.40 (t, J = 11.73 Hz, 1H), 7.43 (d, J = 8.22 Hz, 1H), 7.58 (s, 1H), 7.70 (d, J = 8.68 Hz, 1H), 8.08 (s, 1H), 8.81 (brs, 1H), 8.90 (brs, 1H), 9.13 (brs, 1H), (brs, 1H), 9.28 (brs, 1H). |
| 407 | 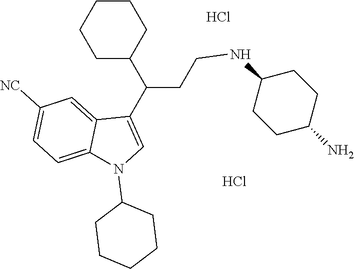 [SMILES: N#Cc1ccc2c(c1)c(C(CCN[C@H]1CC[C@H](N)CC1)C1CCCCC1)cn2C1CCCCC1 ; Cl ; Cl] | ¹H NMR (400 MHz, DMSO-d₆): δ 0.83-0.88 (m, 2H), 0.98- 1.04 (m, 1H), 1.08-1.16 (m, 2H), 1.19-1.32 (m, 6H), 1.36- 1.58 (m, 6H), 1.66-1.74 (m, 4H), 1.77-1.82 (m, 4H), 1.92 (brs, 6H), 1.82 (m, 4H), 2.05-2.09 (m, 2H), 2.75 (brs, 1H), 2.82-2.90 (m, 3H), 4.40 (t, J = 11.99 Hz, 1H), 7.44 (d, J = 8.63 Hz, 1H), 7.53 (s, 1H), 7.71 (d, J = 8.68 Hz, 1H), 7.88 (brs, 3H), 8.08 (s, 1H), 8.90 (brs, 1H), 8.59 (brs, 1H), (brs, 1H), 8.73 (brs, 1H). |
| 408 | 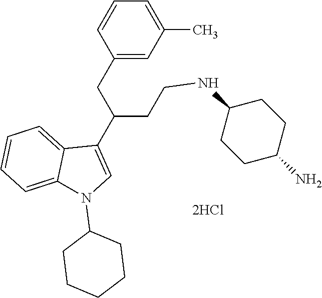 [SMILES: Cc1cccc(CC(CCN[C@H]2CC[C@H](N)CC2)c2cn(C3CCCCC3)c3ccccc23)c1 ; "2HCl"] | ¹H NMR (400 MHz, DMSO-d₆): δ 1.22-1.30 (m, 6H), 1.33- 1.54 (m, 2H), 1.65-1.71 (m, 3H), 1.84 (d, J = 11.84 Hz, 2H), 1.91-2.06 (m, 8H), 2.22 (s, 3H), 2.70-2.88 (m, 5H), 2.98 (dd, J_{1,2} = 6.6.92 Hz, J_{1,3} = 13.20 Hz, 1H), 3.25 (brs, 1H), 4.24-4.30 (m, 1H), 6.92-7.01 (m, 4H), 7.10 (t, J = 7.45 Hz, 1H), 7.34 (s, 1H), 7.47 (d, J = 8.28 Hz, 1H), 7.62 (d, J = 7.81 Hz, 1H), 7.99 (brs, 3H), 8.59 (brs, 1H), 8.86 (brs, 1H). |
| 409 | 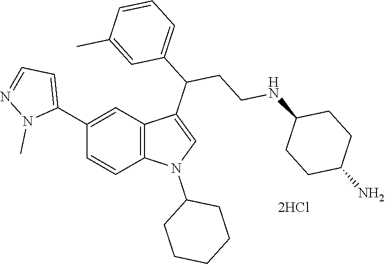 [SMILES: Cc1cccc(C(CCN[C@H]2CC[C@H](N)CC2)c2cn(C3CCCCC3)c3ccc(-c4ccnn4C)cc23)c1 ; "2HCl"] | ¹H NMR (400 MHz, DMSO-d₆): δ 1.22-1.42 (m, 5H), 1.46- 1.56 (m, 2H), 1.70-1.87 (m, 5H), 1.95-2.04 (m, 6H), 2.24 (s, 3H), 2.24 (s, 3H), 2.42-2.46 (m, 1H), 2.79 (brs, 1H), 2.92 (brs, 3H), 3.73 (s, 3H), 4.30-4.37 (m, 2H), 6.24 (s, 1H), 6.92-9.98 (m, 1H), 7.14-7.18 (m, 4H), 7.42 (d, J = 1.59 Hz, 1H), 7.49 (s, 1H), 7.27 (d, J = 8.56 Hz, 1H), 7.61 (s, 1H), 7.99 (brs, 3H), 9.03 (brs, 1H), 9.14 (brs, 1H). |
| 410 | 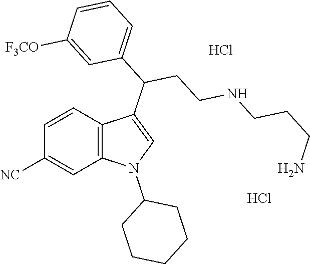 [SMILES: N#Cc1ccc2c(C(CCNCCCN)c3cccc(OC(F)(F)F)c3)cn(C3CCCCC3)c2c1 ; Cl ; Cl] | ¹H NMR (400 MHz, DMSO-d₆): δ 941-9.30 (m, 2H), 8.17- 8. [symbol] (m, 5H), 7.57 (d, J = 7.05 Hz, 1H), 7.37 (d, J = 9.9 Hz, 3H), 7.26 (d, J = 6.85 Hz, 1H), 7.16 (S, 1H), 4.54-4.47 (m, 2H), 2.99-2.80 (m, 7H), 1.94-1.70 (m, 10H), 1.60-1.42 (m, 2H), 1.40-1.22 (m, 1H). |
| 412 | 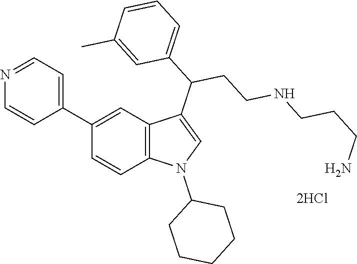 [SMILES: Cc1cccc(C(CCNCCCN)c2cn(C3CCCCC3)c3ccc(-c4ccncc4)cc23)c1 ; "2HCl"] | ¹H NMR (400 MHz, DMSO-d₆): δ 1.22-1.33 (m, 1H), 1.48- 1.57 (m, 2H), 1.72 (d, J = 12.67 Hz, 1H), 1.77-1.87 (m, 4H), 1.91-1.97 (m, 4H), 2.25 (s, 3H), 2.24 (s, 3H), 2.43-2.46 (m, 1H), 2.86-2.87 (m, 1H), 2.89-2.92 (m, 3H), 2.94-2.96 (m, 2H), 4.40 (t, J = 11.78 Hz, 1H), 4.48 (t, J = 8.11 Hz, 1H), 6.98 (d, J = 7.29 Hz, 1H), 7.16-7.24 (m, 3H), 7.66 (s, 1H), 7.70-7.75 (m, 1H), 7.96 (brs, 3H), 8.19(s, 1H), 8.25 (brs, 2H), 8.78 (d, J = 5.94 Hz, 1H), 9.21 (brs, 1H), 9.31(brs, 1H). |
| 413 | 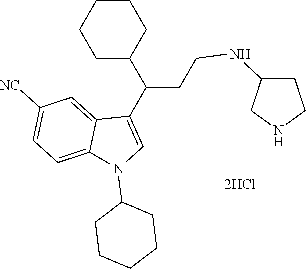 [SMILES: N#Cc1ccc2c(c1)c(C(CCNC1CCNC1)C1CCCCC1)cn2C1CCCCC1] | ¹H NMR (400 MHz, DMSO-d₆): δ 0.84-0.87 (m, 2H), 0.97- 1.03 (m, 1H), 1.08-1.14 (m, 2H), 1.17-1.30 (m, 2H), 1.48- 1.57 (m, 6H), 1.64-1.68 (m, 3H), 1.76-1.79 (m, 4H), 1.81- 1.92 (m, 2H), 1.95-2.21 (m, 4H), 2.62 (brs, 1H), 2.77 (brs, 1H), 2.98 (s, 1H), 3.16 (brs, 1H), 3.78 (brs, 1H), 4.40-4.42 (m, 1H), 7.43 (d, J = 8.44 Hz, 1H), 7.57 (d, J = 7.13 Hz, 1H), 7.70 (d, J = 8.61 Hz, 1H), 8.09 (s, 1H), 9.01 (brs, 1H), 9.30 (brs, 2H), 9.48 (brs, 1H), 9.61(brs, 1H). |
| 414 | 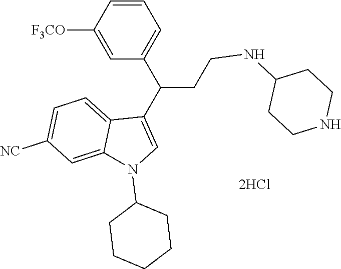 [SMILES: N#Cc1ccc2c(C(CCNC3CCNCC3)c3cccc(OC(F)(F)F)c3)cn(C3CCCCC3)c2c1] | ¹H NMR (400 MHz, DMSO-d6) δ 9.57 (brs, 1H), 9.39 (brs, 1H), 8.95 (brs, 1H), 8.80 (brs, 1H), 8.18 (s, 1H), 8.01 (s, 1H), [symbol] 7.61-7.52 (m, 1H), 7.41-7.32 (m, 3H), 7.30-7.21 (m, 1H), 7.20-7.10 (m, 1H), 4.60-4.42 (m, 2H), 3.55-3.40 (m, 1H), 3.10-2.98 (m, 1H), 2.92-2.80 (m, 5H), 2.15-2.02 (m, 4H), 2.0 [symbol] 1.90 (m, 3H), 1.88-1.70 (m, 8H), 1.60- 1.45 (m, 2H). |
| 415 | 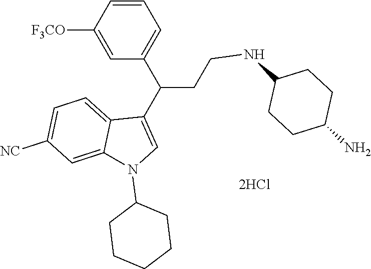 | ¹H NMR (400 MHz, DMSO-d₆): δ 9.11 (brs, 1H), 8.94 (brs, 1H), 8.18 (s, 1H), 7.96-7.93 (m, 4H), 7.59 (d, J = 8.28 Hz, 1H) [symbol] 7.43-7.38 (m, 3H), 7.28 (d, J = 8.33 Hz, 1H), 7.16 (d, J = 8.22 Hz, 1H), 4.53-4.47 (m, 1H), 2.97-2.94 (m, 3H), 2.88-2.82 (m, [symbol] 2H), 2.32-2.30 (m, 1H), 2.10-1.90 (m, 7H), 1.88-1.70 (m, 5H [symbol] 1.60-1.45 (m, 2H), 1.40-1.22 (m, 6H). |
| 416 | 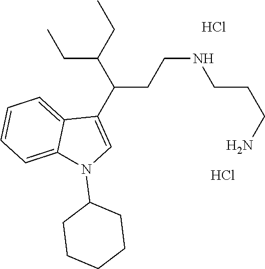 [SMILES: CCC(CC)C(CCNCCCN)c1cn(C2CCCCC2)c2ccccc12 ; Cl ; Cl] | ¹H NMR (400 MHz, DMSO-d₆): δ 0.79-0.97 (m, 6H), 1.13- 1.32 (m, 4H), 1.34-1.37 (m, 1H), 1.44-1.50 (m, 3H), 1.72 (t, J = 12.25 Hz, 3H), 1.79-1.86 (m, 4H), 1.92 (brs, 2H), 2.01- 2.0392 (m, 2H), 2.56-2.59 (m, 1H), 2.75 (brs, 1H), 2.85 (brs, 3H), 2.93-2.98 (m, 1H), 4.28 (t, J = 11.83 Hz, 1H), 6.97 (t, J = 7.30 Hz, 1H), 7.10 (t, J = 7.44 Hz, 1H), 7.34 (s, 1H), 7.48 (dd, J_{1,2} = 7.12 Hz, J_{1,3} = Hz, 14.82 Hz, 2H), 7.90 (brs, 3H), 8.77 (brs, 1H), 8.89 (brs, 1H). |
| 417 | 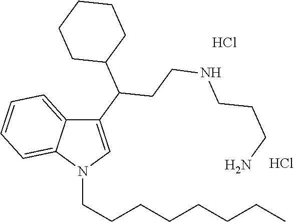 [SMILES: CCCCCCCCn1cc(C(CCNCCCN)C2CCCCC2)c2ccccc21 ; Cl ; Cl] | ¹H NMR (400 MHz, DMSO-d₆): δ 0.79-0.96 (m, 6H), 1.00- 1.22 (m, 14H), 1.50-1.1.57 (m, 5H), 1.65-1.73 (m, 4H), 1.75-1.87 (m, 3H), 2.01-2.11 (m, 2H), 2.56-2.59 (m, 1H), 2.73-2.74 (m, 2H), 2.82-2.86 (m, 4H), 4.06-4.15 (m, 2H), 6.97 (t, J = 6.97 Hz, 1H), 7.09 (t, J = 7.36 Hz, 1H), 7.14 (s, 1H), 7.40 (d, J = 8.26 Hz, 1H), 7.53 (d, J = 7.91 Hz, 1H), 7.88 (brs, 3H), 8.77 (brs, 2H). |
| 418 | 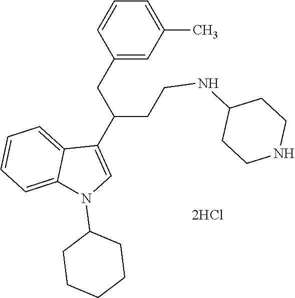 [SMILES: Cc1cccc(CC(CCNC2CCNCC2)c2cn(C3CCCCC3)c3ccccc23)c1] | ¹H NMR (400 MHz, DMSO-d₆): δ 1.22-1.29 (m, 1H), 1.44- 1.54 (m, 2H), 1.65-1.71 (m, 5H), 1.83 (d, J = 10.10 Hz, 2H), 1.89-2.06 (m, 6H), 2.22 (s, 3H), 2.66-2.69 (m, 1H), 2.80- 2.86 (m, 6H), 2.97-3.08 (m, 4H), 3.13 (brs, 1H), 3.26 (s, 2H), 4.25-4.30 (m, 1H), 6.92-7.01 (m, 4H), 7.08-7.13 (m, 2H), 7.35 (s, 1H), 7.47 (d, J = 8.16 Hz, 1H), 7.63 (d, J = 7.83 Hz, 1H), 8.77 (brs, 3H), 9.03 (brs, 1H). |
| 419 | 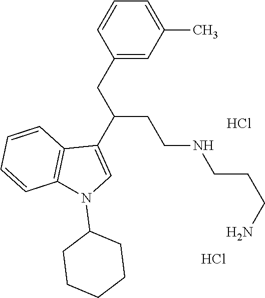 [SMILES: Cc1cccc(CC(CCNCCCN)c2cn(C3CCCCC3)c3ccccc23)c1 ; Cl ; Cl] | ¹H NMR (400 MHz, DMSO-d₆): δ 1.22-1.26 (m, 1H), 1.47- 1.53 (m, 2H), 1.69-1.71 (m, 4H), 1.81-1.88 (m, 6H), 1.99- 2.01 (m, 2H), 2.21 (s, 3H), 2.66-2.83 (m, 9H), 2.96-3.01 (m, 2H), 3.05-3.06 (m, 1H), 3.13-3.14 (m, 1H), 3.23 (brs, 1H), 4.26-4.29 (m, 1H), 6.95-7.00 (m, 4H), 7.09 (t, J = 7.22 Hz, 2H), 7.36 (s, 1H), 7.45 (d, J = 8.11 Hz, 1H), 7.61 (d, J = 7.74 Hz, 1H), 7.98 (s, 1H), 8.08 (brs, 3H), 8.86 (brs, 1H). 9.11 (brs, 1H). |
| 420 | 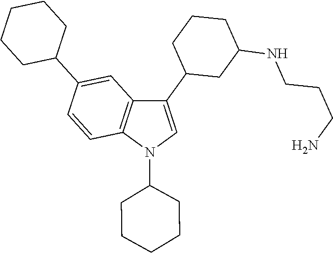 [SMILES: NCCCNC1CCCC(c2cn(C3CCCCC3)c3ccc(C4CCCCC4)cc23)C1] | ¹H NMR (400 MHz, DMSO-d₆): δ 1.20-1.27 (m, 4H), 1.31- 1.53 (m, 9H), 1.61-1.67 (m, 6H), 1.76-1.82 (m, 8H), 1.87- 1.98 (m, 4H), 2.03 (d, J = 11.66 Hz, 1H), 2.77-2.94 (m, 6H), 2.97 (brs, 1H), 3.13 (brs, 1H), 3.18 (brs, 1H), 4.18 (t, J = 11.51 Hz, 1H), 6.96 (d, J = 8.24 Hz, 1H), 7.11 (s, 1H), 7.29- 7.33 (m, 2H). |
| 421 | 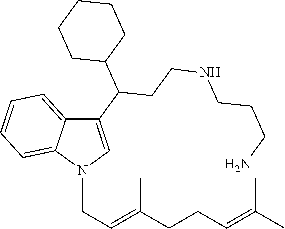 [SMILES: CC(C)=CCC/C(C)=C/Cn1cc(C(CCNCCCN)C2CCCCC2)c2ccccc21] | ¹H NMR (400 MHz, DMSO-d₆): δ 0.84-0.88 (m, 2H), 0.97- 1.22 (m, 5H), 1.40-1.47 (m, 2H), 1.50-1.57 (m, 10H), 1.61- 1.63 (m, 2H), 1.72-1.87 (m, 2H), 1.99-2.04 (m, 5H), 2.32 (s, 1H), 2.40-2.42 (m, 2H), 2.62-2.70 (m, 3H), 3.16 (s, 1H), 4.70 (d, J = 6.53 Hz, 2H), 5.01(s, 1H), 5.27 (t, J = 6.38 Hz, 1H), 6.94 (t, J = 7.32 Hz, 1H), 7.01 (s, 1H), 7.06 (t, J = 7.47 Hz, 1H), 7.30 (d, J = 7.99 Hz, 1H), 7.50 (t, J = 7.89 Hz, 1H). |
| 422 | 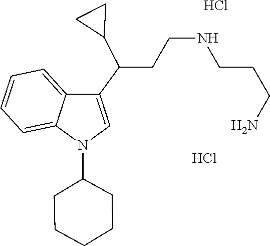 [SMILES: NCCCNCCC(c1cn(C2CCCCC2)c2ccccc12)C1CC1 ; Cl ; Cl] | ¹H NMR (400 MHz, DMSO-d₆): δ 0.03-0.05 (m, 1H), 0.16- 0.18 (m, 1H), 0.31-0.32 (m, 1H), 0.51-0.52 (m, 1H), 1.12 (brs, 1H), 1.19-1.26 (m, 1H), 1.41-1.51 (m, 2H), 1.63-1.69 (m, 3H), 1.80-1.82 (m, 4H), 1.91 (d, J = 10.45 Hz, 2H), 2.08-2.10 (m, 2H), 2.20-2.25 (m, 1H), 2.74-2.82 (m, 3H), 2.88 (t, J = 7.34 Hz, 3H), 3.13 (s, 5H), 4.23 (t, J = 12.23 Hz, 1H), 6.96 (t, J = 7.49 Hz, 1H), 7.08 (t, J = 7.15 Hz, 1H), 7.26 (s, 1H), 7.29 (t, J = 6.69 Hz, 1H), 7.43 (d, J = 8.34 Hz, 1H), 7.53 (d, J = 7.88 Hz, 1H). |
| 423 | 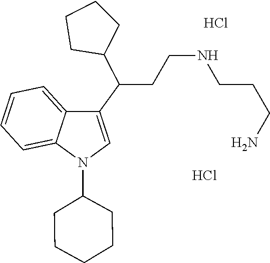 [SMILES: NCCCNCCC(c1cn(C2CCCCC2)c2ccccc12)C1CCCC1 ; Cl ; Cl] | ¹H NMR (400 MHz, DMSO-d₆): δ 1.01-1.06 (m, 1H), 1.19- 1.23 (m, 2H), 1.26-1.70 (m, 7H), 1.73-1.76 (m, 3H), 1.82- 1.95 (m, 9H), 2.02-2.10 (m, 2H), 2.13-2.24 (m, 1H), 2.66- 2.75 (m, 2H), 2.83 (brs, 4H), 4.24-4.30 (m, 1H), 6.96 (t, J = 7.41 Hz, 1H), 7.08 (t, J = 7.29 Hz, 1H), 7.30 (s, 1H), 7.45 (d, J = 8.28 Hz, 1H), 7.55 (d, J = 7.88 Hz, 1H), 7.96 (brs, 3H), 8.85 (brs, 2H). |
| 424 | 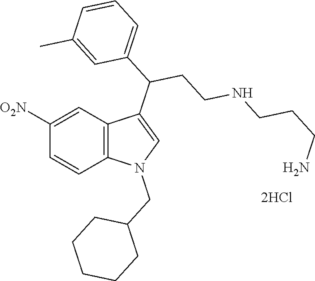 [SMILES: Cc1cccc(C(CCNCCCN)c2cn(CC3CCCCC3)c3ccc([N+](=O)[O-])cc23)c1] | ¹H NMR (400 MHz, DMSO-d₆): δ 0.97-1.03 (m, 1H), 1.13- 1.22 (m, 3H), 1.45-1.50 (m, 2H), 1.53-1.66 (m, 3H), 1.80- 1.81 (m, 1H), 1.91-1.97 (m, 2H), 2.24 (s, 3H), 2.31-2.39 (m, 2H), 2.77 (brs, 1H), 2.88-2.95 (m, 1H), 4.04-4.10 (m, 1H), 4.45 (t, J = 7.53 Hz, 1H), 6.99 (d, J = 6.45 Hz, 1H), 7.14- 7.21 (m, 3H), 7.66 (d, J = 9.13 Hz, 1H), 7.71 (s, 1H), 7.96 (dd, J_{1,2} = 8.28 Hz, J_{1,3} = 9.13 Hz, 1H), 8.03 (brs, 3H), 8.34 (s, 1H), 9.22 (brs, 2H). |
| 425 | 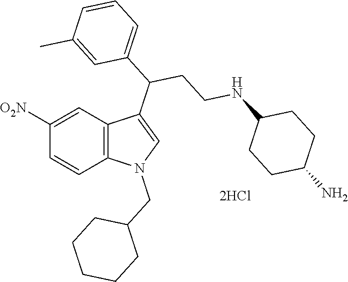 | ¹H NMR (400 MHz, DMSO-d₆): δ 0.93-1.02 (m, 2H), 1.11- 1.22 (m, 5H), 1.26-1.39 (m, 4H), 1.46-1.49 (m, 2H), 1.55- 1.75 (m, 3H), 1.80-1.82 (m, 1H), 1.91-2.04 (m, 4H), 2.24 (s, 3H), 2.79 (brs, 1H), 2.88-2.99 (m, 3H), 4.09 (d, J = 6.88 Hz, 1H), 4.43 (t, J = 8.00 Hz, 1H), 7.00 (d, J = 6.47 Hz, 1H), 7.14-7.22 (m, 3H), 7.66-7.68 (m, 2H), 7.95 (brs, 3H), 7.97 (d, J = 9.16 Hz, 1H), 8.35 (s, 1H), 8.90 (brs, 2H). |
| 426 | 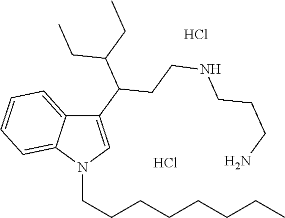 [SMILES: CCCCCCCCn1cc(C(CCNCCCN)C(CC)CC)c2ccccc21 ; Cl ; Cl] | ¹H NMR (400 MHz, DMSO-d₆): δ 0.78-0.86 (m, 9H), 1.17- 1.25 (m, 13H), 1.27-1.39 (m, 2H), 1.47-1.48 (m, 1H), 1.70- 1.73 (m, 2H), 1.89-1.93 (m, 2H), 2.05-2.07 (m, 2H), 2.57 (brs, 1H), 2.72 (brs, 1H), 2.80-2.96 (m, 5H), 4.03-4.17 (m, 2H), 6.96 (t, J = 7.49 Hz, 1H), 7.08 (t, J = 7.43 Hz, 1H), 7.26 (s, 1H), 7.39 (d, J = 8.19 Hz, 1H), 7.51 (d, J = 7.86 Hz, 1H), 8.15 (brs, 3H), 9.18 (brs, 1H), 9.22 (brs, 1H). |
| 427 | 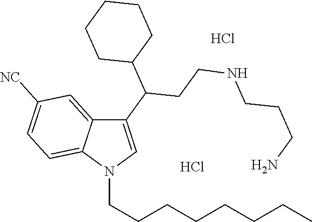 [SMILES: CCCCCCCCn1cc(C(CCNCCCN)C2CCCCC2)c2cc(C#N)ccc21 ; Cl ; Cl] | ¹H NMR (400 MHz, DMSO-d₆): δ 0.78-0.88 (m, 5H), 0.97- 1.03 (m, 1H), 1.17-1.25 (m, 13H), 1.47-1.57 (m, 5H), 1.65- 1.75 (m, 3H), 1.84 (brs, 3H), 2.02-2.12 (m, 2H), 2.71 (brs, 1H), 2.84 (brs, 5H), 4.14-4.23 (m, 2H), 7.40-7.45 (m, 2H), 7.63 (d, J = 8.56 Hz, 1H), 7.92 (brs, 3H), 8.10 (s, 1H), 8.87 (brs, 2H). |
| 428 | 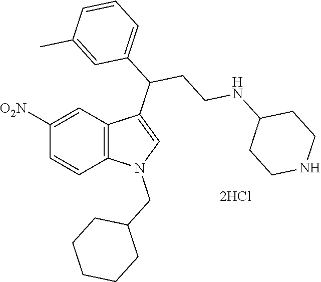 [SMILES: Cc1cccc(C(CCNC2CCNCC2)c2cn(CC3CCCCC3)c3ccc([N+](=O)[O-])cc23)c1] | ¹H NMR (400 MHz, DMSO-d₆): δ 0.84-1.01 (m, 2H), 1.12- 1.23 (m, 4H), 1.46-1.52 (m, 2H), 1.54-1.69 (m, 3H), 1.76- 1.98 (m, 3H), 2.06 (t, J = 11.78 Hz, 2H), 2.24 (s, 3H), 2.49 (brs, 1H), 2.57 (brs, 1H), 2.85-3.09 (m, 4H), 3.22 (brs, 1H), 3.35-3.37 (m, 1H), 4.03-4.13 (m, 2H), 4.34 (t, J = 7.31 Hz, 1H), 7.00 (d, J = 6.29 Hz, 1H), 7.15-7.21 (m, 3H), 7.66 (d, J = 9.05 Hz, 1H), 7.71 (s, 1H), 7.96 (d, J = 9.12 Hz, 1H), 8.18 (brs, 3H), 8.34 (s, 1H), 10.57 (brs, 1H). |
| 429 | 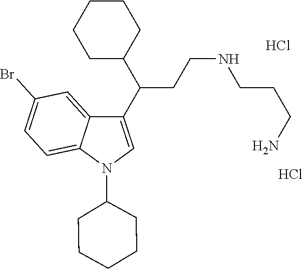 [SMILES: NCCCNCCC(c1cn(C2CCCCC2)c2ccc(Br)cc12)C1CCCCC1 ; Cl ; Cl] | ¹H NMR (400 MHz, DMSO-d₆): δ 0.83-0.98 (m, 2H), 1.00- 1.26 (m, 5H), 1.46-1.57 (m, 5H), 1.62-1.91 (m, 11H), 2.04- 2.10 (m, 2H), 2.55 (s, 1H), 2.66-2.85 (m, 6H), 4.28 (t, 1H), 7.18 (d, J = 8.58 Hz, 1H), 7.38 (s, 1H), 7.47 (d, J = 8.76 Hz, 1H), 7.67 (s, 1H), 8.00 (brs, 3H), 8.99 (brs, 2H). |
| 430 | 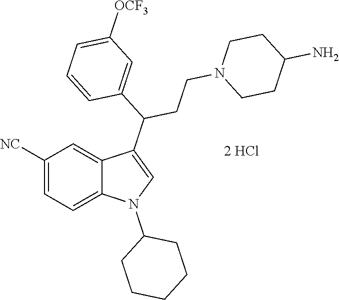 [SMILES: N#Cc1ccc2c(c1)c(C(CCN1CCC(N)CC1)c1cccc(OC(F)(F)F)c1)cn2C1CCCCC1] | ¹H NMR (400 MHz, DMSO-d₆) δ 9.05 (s, 1H), 8.90 (s, 1H), 8.00 (s, 2H) 7.92 (s, 3H) 7.84 (s, 2H) 7.72 (d, J = 8.8 Hz, 2H), 7.48-7.40 (m, 6H), 7.18 (d, J = 6.4 Hz, 2H), 4.51 (t, J = 7.6 Hz, 2H), 4.42 (s, 1H), 2.96 (s, 2H), 2.82 (s, 2H), 2.00 (s, 3H), 1.94 (s, 4H), 1.83 (t, J = 13.1 Hz, 6H), 1.76 (s, 1H), 1.72 (d, J = 11.8 Hz, 3H), 1.50 (d, J = 13.0 Hz, 3H), 1.37- 1.26 (m, 6H). |
| 431 | 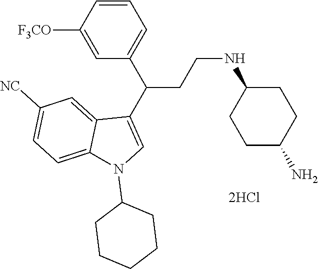 [SMILES: N#Cc1ccc2c(c1)c(C(CCN[C@H]1CC[C@H](N)CC1)c1cccc(OC(F)(F)F)c1)cn2C1CCCCC1 ; "2HCl"] | ¹H NMR (400 MHz, DMSO-d₆) δ 10.80 (s, 1H), 8.23 (s, 2H), 7.99 (d, J = 28.0 Hz, 1H), 7.90 (s, 1H), 7.71 (d, J = 8.6 Hz, 1H), 7.56-7.34 (m, 3H), 7.18 (s, 1H), 4.50-4.20 (m, 2H), 3.55 (d, J = 2.0 Hz, 4H), 3.37 (s, 9H), 2.76 (dd, J = 70.3, 27.2 Hz, 5H), 2.06 (d, J = 13.1 Hz, 3H), 1.96-1.66 (m, 7H), 1.51 (d, J = 13.8 Hz, 2H), 1.25 (d, J = 21.8 Hz, 3H), 0.84 (d, J = 7.4 Hz, 2H). |
| 434 | 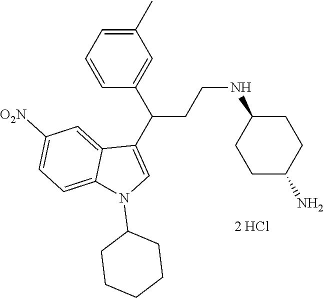 [SMILES: Cc1cccc(C(CCN[C@H]2CC[C@H](N)CC2)c2cn(C3CCCCC3)c3ccc([N+](=O)[O-])cc23)c1 ; "2 HCl"] | ¹H NMR (400 MHz, DMSO-d₆) δ 9.01 (s, 1H), 8.91 (s, 1H), 8.36 (d, J = 2.2 Hz, 1H), 8.08-7.91 (m, 4H), 7.88-7.61 (m, 3H), 7.18 (d, J = 10.2 Hz, 4H), 7.00 (d, J = 6.9 Hz, 1H), 4.43 (q, J = 9.0, 8.0 Hz, 4H), 3.56 (s, 4H), 2.87 (d, J = 53.8 Hz, 6H), 2.25 (s, 6H), 1.98 (d, J = 24.8 Hz, 6H), 1.77 (d, J = 53.6 Hz, 5H), 1.52 (d, J = 13.2 Hz, 3H), 1.31 (dd, J = 24.7, 12.5 Hz, 6H). |
| 433 | 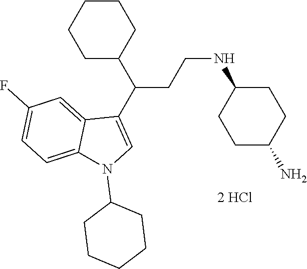 [SMILES: N[C@H]1CC[C@H](NCCC(c2cn(C3CCCCC3)c3ccc(F)cc23)C2CCCCC2)CC1 ; "2 HCl"] | ¹H NMR (400 MHz, DMSO-d₆) δ 8.64 (s, 1H), 7.91 (s, 3H), 7.49 (dd, J = 9.0, 4.5 Hz, 1H), 7.42-7.15 (m, 3H), 6.93 (t, J = 9.0 Hz, 2H), 4.27 (d, J = 12.4 Hz, 3H), 2.91 (s, 3H), 2.04 (d, J = 7.9 Hz, 2H), 1.92 (d, J = 10.8 Hz, 5H), 1.78-1.49 (m, 9H), 1.39 (d, J = 66.0 Hz, 8H), 1.25 (d, J = 21.8 Hz, 5H), 1.14-0.63 (m, 6H). |
| 434 | 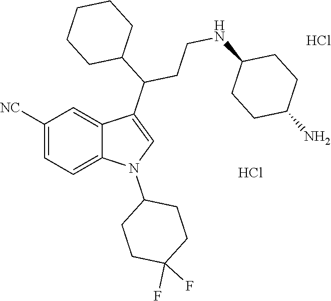 [SMILES: N#Cc1ccc2c(c1)c(C(CCN[C@H]1CC[C@H](N)CC1)C1CCCCC1)cn2C1CCC(F)(F)CC1 ; Cl ; Cl] | ¹H NMR (400 MHz, DMSO-d₆) δ 8.94 (s, 1H), 8.83 (d, J = 11.8 Hz, 1H) 8.11 (s, 2H) 8.00 (s, 3H), 7.74 (d, J = 8.7 Hz, 2H), 7.58 (s, 2H), 7.49 (d, J = 8.6 Hz, 2H), 4.70 (s, 1H), 2.88 (s, 3H), 2.73 (s, 1H), 2.19 (s, 3H), 2.16-2.05 (m, 7H), 2.00 (s, 5H), 1.95 (s, 2H), 1.93 (d, J = 8.8 Hz, 4H), 1.79 (d, J = 12.4 Hz, 2H), 1.67 (d, J = 11.7 Hz, 2H), 1.56 (s, 3H), 1.39-1.27 (m, 6H), 1.14 (t, J = 14.1 Hz, 4H), 0.99 (d, J = 12.7 Hz, 2H), 0.84 (d, J = 12.4 Hz, 4H). |
| 435 | 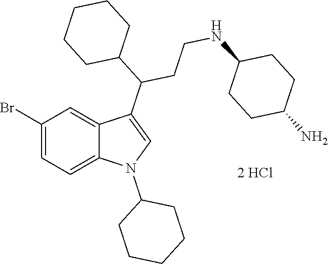 [SMILES: N[C@H]1CC[C@H](NCCC(c2cn(C3CCCCC3)c3ccc(Br)cc23)C2CCCCC2)CC1 ; "2 HCl"] | ¹H NMR (400 MHz, DMSO-d₆) δ 8.87 (s, 1H), 8.70 (s, 1H), 7.96 (s, 3H), 7.67 (d, J = 2.0 Hz, 1H), 7.48 (d, J = 8.8 Hz, 1H), 7.37 (d, J = 2.3 Hz, 1H), 7.27-7.12 (m, 1H), 4.29- 3.94 (m, 2H), 3.56 (s, 3H), 3.16 (s, 1H), 2.89 (d, J = 10.9 Hz, 2H), 2.74 (d, J = 16.8 Hz, 2H), 1.99 (d, J = 49.4 Hz, 5H), 1.86-1.33 (m, 12H), 1.35-0.64 (m, 10H). |
| 436 | 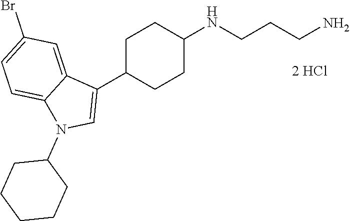 [SMILES: NCCCNC1CCC(c2cn(C3CCCCC3)c3ccc(Br)cc23)CC1] | ¹H NMR (400 MHz, DMSO-d₆) δ 7.99 (s, 1H), 7.76 (d, J = 17.1 Hz, 2H), 7.51-7.40 (m, 2H), 7.28 (s, 1H), 7.19 (d, J = 8.7 Hz, 2H), 4.27 (s, 1H), 3.03 (s, 3H), 2.91 (s, 2H), 2.15 (s, 2H), 2.08-1.96 (m, 5H), 1.89 (s, 3H), 1.82 (d, J = 13.0 Hz, 5H), 1.69 (d, J = 12.1 Hz, 4H), 1.51 (dd, J = 27.1, 11.6 Hz, 7H), 1.24 (s, 1H). |
| S-2 | 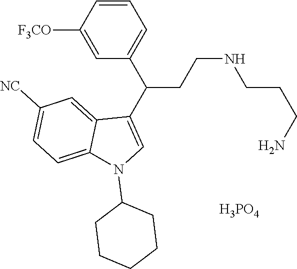 | ¹H NMR (400 MHz, DMSO-d₆) δ 7.96 (s, 1H), 7.74 (d, J = 40.5 Hz, 1H), 7.67 (s, 1H), 7.40 (t, J = 7.3 Hz, 4H), 7.13 (d, J = 6.8 Hz, 1H), 4.40 (d, J = 9.7 Hz, 2H), 2.79 (d, J = 9.0 Hz, 3H), 2.64 (s, 3H), 2.35 (s, 1H), 2.24 (s, 1H), 1.78 (q, J = 31.6, 29.3 Hz, 8H), 1.48 (q, J = 13.5 Hz, 2H), 1.27 (q, J = 12.7, 11.8 Hz, 1H). |
| S-3 | 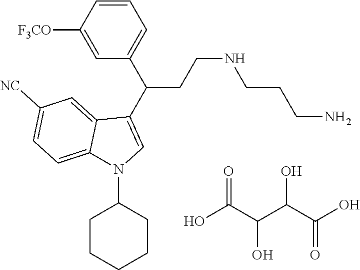 [SMILES: N#Cc1ccc2c(c1)c(C(CCNCCCN)c1cccc(OC(F)(F)F)c1)cn2C1CCCCC1 ; O=C(O)C(O)C(O)C(=O)O] | ¹H NMR (400 MHz, DMSO-d₆) δ 7.97 (s, 1H), 7.80 (s, 1H), 7.70 (s, 1H), 7.41 (m, 4H), 7.16 (s, 1H), 4.42 (s, 2H), 3.89 (s, 6H), 1.87 (d, J = 38.1 Hz, 8H), 1.27 (s, 2H). |
| S-4 | 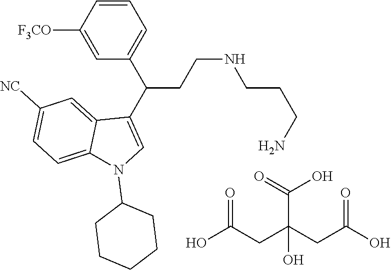 | ¹H NMR (400 MHz, DMSO-d₆) δ 7.97 (s, 1H), 7.71 (d, J = 8.5 Hz, 1H), 7.43 (d, J = 9.6 Hz, 3H), 7.16 (s, 1H), 4.41 (s, 2H), 1.82 (t, J = 41.2 Hz, 8H), 1.49 (s, 2H), 1.25 (d, J = 22.2 Hz, 1H). |
| S-5 | 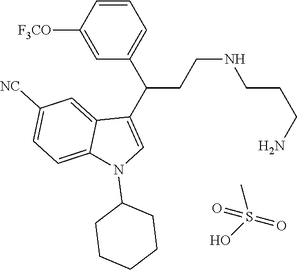 | ¹H NMR (400 MHz, DMSO-d₆) δ 7.97 (s, 1H), 7.79 (s, 1H), 7.70 (d, J = 8.6 Hz, 1H), 7.52-7.28 (m, 4H), 7.15 (s, 1H), 4.42 (d, J = 7.2 Hz, 2H), 2.83 (t, J = 7.2 Hz, 2H), 2.67 (s, 2H), 2.30 (s, 3H), 2.01 (br s, 1H), 2.02-1.59 (m, 8H), 1.50 (d, J = 13.5 Hz, 5H), 1.37 (s, 2H), 1.26 (d, J = 13.7 Hz, 2H). |
| S-6 | 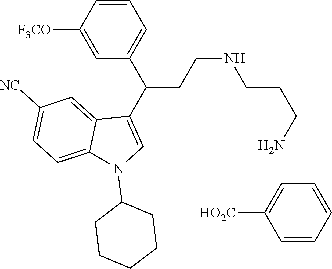 | ¹H NMR (400 MHz, DMSO-d₆) δ 7.92 (d, J = 18.7 Hz, 3H), 7.74 (d, J = 39.2 Hz, 1H), 7.67 (s, 1H), 7.54-7.29 (m, 7H), 7.13 (s, 1H), 4.40 (d, J = 17.1 Hz, 2H), 2.86 (s, 2H), 2.70 (s, 2H), 2.38 (s, 1H), 2.24 (s, 1H), 1.89 (s, 2H), 1.75 (dq, J = 27.5, 13.0 Hz, 5H), 1.48 (d, J = 13.5 Hz, 2H), 1.23 (d, J = 12.6 Hz, 1H). |
| S-7 | 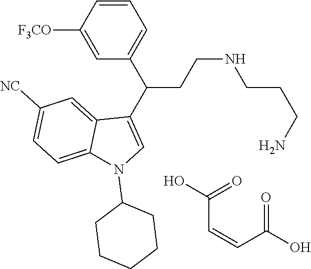 | ¹H NMR (400 MHz, DMSO-d₆) δ 7.99 (s, 1H), 7.79-7.65 (m, 2H), 7.51-7.36 (m, 4H), 7.18 (d, J = 6.9 Hz, 1H), 6.00 (s, 3H), 4.44 (d, J = 9.1 Hz, 2H), 2.83 (t, J = 7.6 Hz, 4H), 1.91 (t, J = 16.8 Hz, 3H), 1.88-1.68 (m, 6H), 1.56-1.43 (m, 2H), 1.26 (td, J = 33.8, 30.5, 22.8 Hz, 2H). |

[symbol]

indicates data missing or illegible when filed

Anti-Infective Activity of the Synthesised Compounds

The compounds as disclosed by the present application have anti-infective activity.

Initial minimal inhibitory concentration (MIC) tests were made on two bacterial strains:
Escherichia coli (ATCC25922)
Staphylococcus aureus (ATCC25923).

The results of these tests are shown in Table XIII.

The MIC of selected compounds was determined against a number of additional strains:

Enterococcus faecalis (ATCC29212)

Pseudomonas aeruginosa (ATCC27853)

Staphylococcus aureus subsp. aureus (ATCC29213)

Klebsiella pneumoniae subsp. pneumoniae (ATCC13883)

Streptococcus pneumoniae (ATCC33400)

Haemophilus influenzae (ATCC49766)

Neisseria meningitidis (ATCC13077)

Listeria monocytogenes (ATCC15313)

Legionella pneumophila subsp. pneumophila (ATCC33152)

Mycobacterium bovis BCG (ATCC19210)

The results of these tests are shown in Table XIV.

Minimal Inhibitory Concentration (MIC)

MIC values were determined using the standard broth microdilution procedure based on the guidelines by the Clinical and Laboratory Standards Institute (CLSI). Briefly, the compounds were dissolved in DMSO to 10 mM. They were diluted in cation-adjusted Mueller-Hinton broth (CAMHB) to four times the highest concentration tested. A serial two-fold dilution in CAMHB was done in microdilution plates. The inoculum of bacterial strain to be tested was prepared by making a suspension of colonies from an 18 to 24 hours old plate in CAMHB. The inoculum was diluted so that, after inoculation, each well contained approximately 5×10⁵ CFU/mL. To a volume of 50 μl compound in CA 3 an equal volume of inoculum was added. The tray was sealed in a plastic bag and incubated at 35° C. for 16 to 20 hours. To aid in the detection of growth the dye resazurin was added to a final concentration 0.001% and incubated at room temperature for 1 h. Reduction of resazurin, and therefore bacterial growth, was seen as a change from blue to pink. The MIC is the lowest concentration of compound that completely inhibits growth of the organism. The method used is described in detail in: Methods for Dilution Antimicrobial Susceptibility Tests for Bacteria That Grow Aerobically; Approved Standard—Ninth Edition. CLSI document M07-A9. Wayne, Pa.: Clinical and Laboratory Standards Institute; 2012.

Inhibition of Bacterial RNaseP Activity.

The assay is based on how much the cleavage of the model substrate pATSerUG by E. coli RNase P RNA, M1 RNA, is inhibited by the compound.

The substrate pATSerUG is a 45 nt long model substrate encompassing the 5′ leader, the amino acid acceptor stem and the T-stem/loop structure of the E. coli tRNA^{Ser}Su1 precursor. It was purchased from Dharmacon/GE Healthcare, and labelled with ³²P at the 5′ end with [γ-³²P]ATP according to standard procedures, and purified by electrophoresis on a denaturing polyacrylamide gel.

The M1 RNA was generated by T7 in vitro transcription using a PCR product with the M1 RNA gene as template.

The compound to be tested was dissolved in assay buffer (see below). Assay buffer was added to a theoretical concentration of up to 10 mM. After vortexing and incubation at room temperature for 30 minutes the undissolved compound was removed by centrifugation (17,000×g 10 min). The concentration of compound in the supernatant was determined spectroscopically by measuring the absorbance at a wavelength where the compound had an absorbance maximum. The calibration curve was made from known concentrations of the compound dissolved in DMSO.

The cleavage reaction was performed in assay buffer (50 mM Tris-HCl, pH 7.9, 1 m MNH₄Cl, 10 mM MgCl₂, 5% PEG6000, 10 mM spermidine).

M1 RNA was diluted to 10 times the concentration to be used in assay buffer and preincubated at 37° C. for 10 min to allow proper folding. The final concentration of M1 RNA was determined for each batch of enzyme, and was the concentration that gave approximately 50% cleavage of the substrate in a 10 min reaction. The folded M1 RNA was mixed with the compound to be tested in a total volume of 9 μl and incubated for an additional 10 min at 37° C. The substrate was preheated separately for 5 min at 37° C. The reaction was started by the addition of 1 μl substrate to the M1 RNA-compound mixture. After 10 min incubation at 37° C. the reaction was stopped by the addition of 20 μl stop solution (10 M urea, 100 mM EDTA, 0.05% bromophenol blue, 0.05% xylene cyanol). The reactions were then heated to 95° C. for 3 min, chilled on ice, the cleavage products were seperated on denaturing 20% polyacrylamide (7 M urea/TBE) gels and detected using a Phosphoimager. The signals were quantitated using the softwares QuantityOne or ImageLab.

Initial Screening for Inhibition of RNase P Activity

To test if any inhibition could be detected for the compound an initial inhibition of RNase P activity was determined. The maximum amount of compound was used, i.e. 8 μl of the supernatant from freshly dissolved compound in assay buffer in a 10 μl cleavage reaction. The degree of inhibition was judged from the normalised cleavage (the ratio between cleavage with compound divided by cleavage without compound). If this ratio was <0.5, the IC50 value was determined (Table XIII).

IC₅₀ Determination.

About 8 different concentrations, generally ranging from maximum concentration for the compound down to 8000 times diluted, were tested for cleavage. The IC50 values and Hill slopes were calculated using the software GraphPad Prism. The determined IC50 values are listed in Table XIV.

TABLE XIII
RNase P inhibition and Antibacterial Efficacy Results

| | | | E. coli | S. aureus | S. aureus |
|---|---|---|---|---|---|
| | Initial | RNase P | ATCC | ATCC | ATCC |
| | screening of | Inhibition | 25922 | 25923 | 29213 |
| | RNase P | IC₅₀ | MIC | MIC | MIC |
| Cmpd | Inhibition | (μM) | (μg/ml) | (μg/ml) | (μg/ml) |
| 2 | | | | | |
| 3 | | | >512 | 32 | |
| 7 | | | | | |
| 8 | 0.96 | | | | |
| 9 | 0.91 | | | | |
| 10 | 0.95 | | | | |
| 11 | 0.72 | | | | |
| 12 | 0.84 | | | | |
| 13 | 0.97 | | | | |
| 14 | 0.78 | | | | |
| 15 | 1.10 | | | | |
| 16 | 0.88 | | | | |
| 17 | 0.94 | | | | |
| 18 | 1.05 | | | | |
| 19 | | | | | |
| 20 | | 4141 | >512 | >512 | |
| 21 | | | | | |
| 22 | | | | | |
| 23 | | | | | |
| 24 | | NI | 46 | 6 | 6 |
| 25 | | | | | |
| 26 | | | | | |
| 27 | | | | | |
| 28 | | | | | |
| 29 | | | | | |
| 30 | | | | | |
| 31 | | | | | |
| 32 | | | | | |
| 33 | | | | | |
| 34 | | | | | |
| 35 | | | | | |
| 36 | | | | | |
| 37 | | | | | |
| 38 | | | | | |
| 39 | | 959 | >512 | 265 | |
| 40 | | | | | |
| 41 | | 980 | >512 | 258 | |
| 42 | | | | | |
| 43 | | | | | |
| 44 | | | | | |
| 45 | | | | | |
| 46 | | | | | |
| 47 | | | | | |
| 49 | | | | | |
| 50 | | | | | |
| 51 | | | | | |
| 52 | | | | | |
| 53 | | | | | |
| 54 | | 100 | | | |
| 55 | | 172 | 264 | 66 | |
| 56 | | 127 | >512 | 64 | |
| 58 | | | | | |
| 59 | | | | | |
| 60 | | | | | |
| 61 | | 182 | 251 | 63 | |
| 62 | | | | | |
| 63 | | | | | |
| 64 | | 58 | >512 | 64 | |
| 65 | | | | | |
| 66 | | 195 | 133 | 33 | |
| 67 | | | | | |
| 68 | | 201 | >512 | 68 | |
| 69 | | 825 | >512 | >512 | |
| 70 | | 1093 | >512 | 250 | |
| 71 | | 269 | >512 | 258 | |
| 72 | | | | | |
| 73 | | | | | |
| 74 | | | | | |
| 75 | | 343 | | | |
| 76 | | 197 | | | |
| 77 | | 140 | | | |
| 78 | | | | | |
| 79 | | 129 | | | |
| 80 | | 41 | 64 | 32 | |
| 81 | 0.02 | | | | |
| 82 | 0.03 | | | | |
| 83 | 0.59 | | | | |
| 84 | 0.32 | 823 | | | |
| 85 | 0.20 | 806 | | | |
| 86 | 0.78 | 242 | | | |
| 87 | 0.30 | 154 | | | |
| 88 | | 842 | | | |
| 89 | | 37 | 32 | 8 | |
| 90 | | 950 | | | |
| 91 | | 1077 | | | |
| 92 | | 45 | 64 | 16 | |
| 93 | | 308 | | | |
| 94 | | 912 | | | |
| 95 | | 1299 | | | |
| 96 | | 911 | | | |
| 97 | | | | | |
| 98 | | 269 | | | |
| 99 | | 18 | >512 | 256 | |
| 100 | | 486 | >512 | 256 | |
| 101 | | 387 | 128 | 128 | |
| 102 | | 950 | | | |
| 103 | | 200 | 128 | 16 | |
| 104 | | 16 | 64 | 8 | |
| 106 | | 1224 | 512 | 512 | |
| 107 | | 1334 | >512 | 512 | |
| 108 | | 2078 | >512 | >512 | |
| 109 | | 3.7 | 128 | 4 | 4 |
| 110 | | 5.6 | 64 | 4 | |
| 111 | | 6.8 | 32 | 4 | |
| 112 | | 68 | 64 | 16 | |
| 113 | | 14 | 512 | 32 | |
| 114 | | 14 | 64 | 8 | |
| 115 | | 145 | 128 | 128 | |
| 116 | | 158 | 128 | 128 | |
| 117 | | 166 | 256 | 256 | |
| 118 | | 39 | 32 | 8 | |
| 119 | | 19 | 32 | 8 | |
| 120 | | 11 | >512 | 2 | |
| 121 | | 8 | 256 | 8 | |
| 122 | | 4 | 4 | 2 | 8 |
| 123 | | 389 | 256 | 64 | |
| 124 | | 149 | 256 | 128 | |
| 125 | | 29 | 256 | 8 | |
| 126 | | 5.8 | >512 | 4 | |
| 127 | | 19 | >512 | 512 | |
| 128 | | | | | |
| 129 | | 13 | >512 | 4 | |
| 130 | | | 64 | 64 | |
| 131 | | 93 | 128 | 32 | |
| 132 | | 191 | >512 | 128 | |
| 133 | | 76 | >512 | >512 | |
| 134 | | 19 | 256 | 4 | |
| 135 | | 8 | 256 | 4 | |
| 136 | | 9 | >512 | 64 | |
| 137 | | 43 | 32 | 8 | |
| 138 | | 95 | 128 | 64 | |
| 139 | | 46 | >512 | 2 | |
| 140 | | 34 | 64 | 8 | |
| 141 | | 13 | 8 | 2 | |
| 142 | | 44 | 32 | 8 | |
| 143 | | 20 | >512 | 2 | |
| 144 | | 7.5 | 64 | 4 | |
| 145 | | 1485 | >512 | 512 | |
| 146 | | 52 | 256 | 8 | |
| 148 | | 160 | 256 | 128 | |
| 149 | | 13 | >512 | 16 | |
| 150 | | 64 | 512 | 8 | |
| 151 | | 45 | 128 | 16 | |
| 153 | | 41 | >512 | 64 | |
| 154 | | 1229 | 256 | 128 | |
| 155 | | NI | >512 | >512 | |
| 156 | | 16 | 32 | 16 | |
| 157 | | 3.4 | 262 | 131 | |
| 158 | | NI | >512 | >512 | |
| 159 | | 210 | >512 | 279 | |
| 160 | | 12 | 67 | 17 | |
| 161 | | 12 | 2 | 2 | 2 |
| 162 | | 9 | 32 | 4 | |
| 163 | | 8.5 | 2 | 1 | 2 |
| 164 | | 9.6 | 8 | 4 | 8 |
| 165 | | 18 | 67 | 34 | |
| 166 | | 7 | 275 | 9 | |
| 167 | | 14 | 65 | 8 | |
| 168 | | 11 | 5 | 2 | |
| 169 | | 21 | 5 | 2 | |
| 170 | | 16 | 4 | 2 | |
| 171 | | 12 | 5 | 2 | |
| 172 | | 10 | 4 | 4 | |
| 173 | | 3 | 4 | 2 | 2 |
| 174 | | 32 | >512 | 150 | |
| 175 | | 59 | >512 | 2 | |
| 176 | | 384 | >512 | >512 | |
| 177 | | 15 | 18 | 9 | |
| 178 | | 20 | 65 | 8 | |
| 179 | | 12 | 63 | 8 | |
| 180 | | 18 | 67 | 17 | |
| 181 | | 11 | 134 | 17 | |
| 182 | | 17 | >512 | 17 | |
| 183 | | 17 | 151 | 9 | |
| 186 | | 3.6 | 4 | 4 | 4 |
| 188 | | 5.8 | >512 | 5 | |
| 189 | | 16 | >512 | 16 | |
| 190 | | 10 | 31 | 15 | |
| 191 | | 7 | 8 | 5 | 8 |
| 197 | | 31 | 8 | 4 | |
| 198 | | 12.7 | >512 | 17 | |
| 199 | | 9.8 | 4 | 2 | 4 |
| 200 | | 5.9 | 59 | 15 | |
| 201 | | 15.4 | 75 | 2 | |
| 202 | | 10.7 | 33 | 2 | |
| 203 | | 115 | >512 | 5 | |
| 204 | | 50 | 4 | 2 | |
| 205 | | 3.2 | >512 | 158 | |
| 206 | | 23 | >512 | 19 | |
| 207 | | 17 | 4 | 4 | |
| 208 | | NI | >512 | >512 | |
| 209 | | 5.4 | 63 | 4 | |
| 210 | | NI | >512 | >512 | |
| 211 | | 20 | 8 | 4 | |
| 213 | | 3 | 296 | 18 | |
| 214 | | 13 | >512 | 10 | |
| 215 | | NI | >128 | 2 | 2 |
| 216 | | 25 | 4 | 4 | |
| 217 | | 133 | 31 | 8 | |
| 218 | | 45 | >512 | 8 | |
| 219 | | 7 | >512 | 10 | |
| 221 | | 3.4 | >512 | 38 | |
| 222 | | 5.7 | 5 | 5 | |
| 223 | | 8 | >512 | 2 | |
| 224 | | 2.9 | 82 | 5 | |
| 225 | | 11.8 | 2 | 2 | 2 |
| 226 | | 28.5 | >512 | 311 | |
| 227 | | NI | >512 | 318 | |
| 228 | | 57 | 40 | 10 | |
| 229 | | 5.4 | 20 | 3 | |
| 230 | | 11 | 67 | 8 | |
| 231 | | 4.4 | >512 | 5 | |
| 232 | | 8.3 | 5 | 2 | |
| 234 | | 7 | 289 | 9 | |
| 235 | | 12 | 126 | 8 | |
| 236 | | 20 | 68 | 9 | |
| 237 | | 9.8 | 4 | 2 | 4 |
| 238 | | 9.2 | >512 | 4 | |
| 239 | | 2.9 | 271 | 4 | |
| 240 | | NI | 289 | 9 | |
| 241 | | 25 | 5 | 2 | |
| 242 | | 6.6 | 5 | 2 | |
| 243 | | 35 | >512 | 17 | |
| 244 | | 14.7 | 2 | 2 | |
| 245 | | 13 | 4 | 1 | 4 |
| 247 | | Ni | >512 | >512 | |
| 255 | | 33 | 66 | 8 | |
| 259 | | 20 | >512 | 267 | |
| 264 | | 298 | >512 | 240 | |
| 265 | | 15 | 2 | 2 | |
| 266 | | 8.5 | 2 | 2 | 2 |
| 268 | | 59 | 30 | 15 | |
| 269 | | 29 | >512 | 8 | |
| 270 | | 135 | 127 | 64 | |
| 272 | | 121 | 68 | 34 | |
| 274 | | 6.53 | 2 | 2 | 4 |
| 275 | | 79 | >128 | >512 | >128 |
| 276 | | 43 | 54 | 54 | |
| 277 | | 229 | 129 | 65 | |
| 278 | | 15 | >512 | 2 | |
| 279 | | 34 | 65 | 8 | |
| 280 | | 103 | 131 | 16 | |
| 281 | | 168 | >512 | 19 | |
| 282 | | 71 | 136 | 34 | |
| 284 | | 316 | >512 | >512 | |
| 285 | | 74 | 147 | 37 | |
| 286 | | 164 | 133 | 66 | |
| 287 | | 15 | 78 | 10 | |
| 288 | | 52 | 4 | 4 | 128 |
| 289 | | 15 | 16 | 2 | 64 |
| 290 | | 61 | 17 | 17 | |
| 291 | | 10 | 10 | 2 | |
| 292 | | 130 | 66 | 33 | |
| 293 | | 70 | 4 | 5 | 8 |
| 295 | | 50 | 17 | 9 | |
| 296 | | NI | >512 | >512 | |
| 297 | | 38 | >128 | 34 | 32 |
| 298 | | NI | >512 | 15 | |
| 299 | | 32 | 134 | 8 | |
| 300 | | | 128 | | 128 |
| 301 | | | 128 | | 128 |
| 302 | | | 128 | | 128 |
| 303 | | | 128 | | 128 |
| 304 | | | >128 | | 64 |
| 305 | | | >128 | | >128 |
| 306 | | | >128 | | 16 |
| 307 | | | 64 | | 64 |
| 308 | | 29 | 4 | | 4 |
| 309 | | 39 | 4 | | 8 |
| 310 | | 18 | 4 | | 4 |
| 311 | | | >128 | | 128 |
| 312 | | | 64 | | 32 |
| 313 | | | >128 | | 16 |
| 314 | | | >128 | | 16 |
| 315 | | | >128 | | 64 |
| 316 | | | 64 | | 64 |
| 317 | | | >128 | | 64 |
| 318 | | | >128 | | 64 |
| 319 | | | 64 | | 32 |
| 320 | | | 128 | | 128 |
| 321 | | | 64 | | 32 |
| 322 | | 120 | >128 | | 16 |
| 323 | | 42 | 4 | | 8 |
| 324 | | | 64 | | 32 |
| 325 | | | 64 | | 64 |
| 326 | | | 128 | | 32 |
| 327 | | | >128 | | 32 |
| 328 | | | 64 | | 32 |
| 329 | | | 64 | | 32 |
| 330 | | | >128 | | 64 |
| 331 | | | 128 | | 32 |
| 332 | | 39 | 8 | | 8 |
| 333 | | 89 | 8 | | 16 |
| 334 | | 68 | 8 | | 8 |
| 335 | | | 64 | | 64 |
| 336 | | | 128 | | 64 |
| 337 | | | 64 | | 32 |
| 338 | | | 128 | | 64 |
| 339 | | | 128 | | 32 |
| 340 | | | 128 | | 64 |
| 341 | | 32 | 4 | | 8 |
| 342 | | | 8 | | 16 |
| 343 | | 64 | 4 | | 8 |
| 344 | | 52 | 4 | | 8 |
| 345 | | 41 | 4 | | 8 |
| 346 | | | 8 | | 8 |
| 347 | | | 16 | | 8 |
| 348 | | 4.5 | 4 | | 4 |
| 349 | | 16 | 4 | | 2 |
| 350 | | | 8 | | 8 |
| 351 | | 16 | 4 | | 4 |
| 352 | | | >128 | | 8 |
| 353 | | 19 | 32 | | 4 |
| 354 | | | >128 | | 128 |
| 655 | | 11 | 4 | | 4 |
| 356 | | 37 | 4 | | 4 |
| 357 | | 10 | 4 | | 4 |
| 358 | | 38 | 4 | | 8 |
| 359 | | | 16 | | 16 |
| 360 | | 7.8 | 4 | | 4 |
| 361 | | | >128 | | >128 |
| 362 | | 8.3 | 4 | | 4 |
| 363 | | 12 | 32 | | 16 |
| 364 | | | >128 | | 8 |
| 365 | | 21 | >128 | | 4 |
| 366 | | | >128 | | 16 |
| 367 | | | >128 | | 64 |
| 368 | | | 8 | | 8 |
| 369 | | | 8 | | 8 |
| 370 | | 8.4 | 4 | | 4 |
| 371 | | 9 | 2 | | 4 |
| 372 | | 9.1 | 4 | | 4 |
| 373 | | 120 | >128 | | 8 |
| 374 | | 53 | >128 | | 16 |
| 375 | | 4.7 | 4 | | 4 |
| 376 | | 40 | 32 | | 16 |
| 377 | | 130 | >128 | | 16 |
| 378 | | 22 | 8 | | 4 |
| 379 | | 34 | 8 | | 8 |
| 380 | | 35 | 4 | | 4 |
| 381 | | 11 | 4 | | 2 |
| 382 | | 20 | 4 | | 4 |
| 383 | | 28 | 4 | | 4 |
| 384 | | 19 | 4 | | 4 |
| 385 | | 13 | 4 | | 4 |
| 386 | | | 8 | | 16 |
| 387 | | | >128 | | 128 |
| 388 | | | >128 | | 16 |
| 389 | | | 32 | | 16 |
| 390 | | | 8 | | 8 |
| 391 | | | 128 | | 64 |
| 392 | | | 8 | | 8 |
| 393 | | 16 | 8 | | 4 |
| 394 | | 15 | 32 | | 8 |
| 395 | | 8.2 | 8 | | 1 |
| 396 | | 12 | 4 | | 2 |
| 397 | | 6 | 8 | | 2 |
| 398 | | 9 | 4 | | 2 |
| 399 | | 6.3 | 4 | | 4 |
| 400 | | | 16 | | 16 |
| 401 | | | 16 | | 16 |
| 402 | | | 16 | | 8 |
| 403 | | 7 | 16 | | 4 |
| 404 | | 4.8 | 8 | | 4 |
| 405 | | | 16 | | 8 |
| 406 | | | 8 | | 8 |
| 407 | | 23 | 8 | | 4 |
| 408 | | 21 | 8 | | 4 |
| 409 | | 39 | 16 | | 4 |
| 410 | | 10 | 4 | | 4 |
| 412 | | 18 | 4 | | 2 |
| 413 | | 20 | 8 | | 4 |
| 414 | | 16 | 8 | | 4 |
| 415 | | 16 | 16 | | 4 |
| 416 | | 26 | 2 | | 4 |
| 417 | | 2.8 | 4 | | 2 |
| 418 | | 17 | 4 | | 8 |
| 419 | | | 8 | | 8 |
| 420 | | 8.4 | 2 | | 2 |
| 421 | | 8.3 | 8 | | 4 |
| 422 | | | 16 | | 32 |
| 423 | | 31 | 2 | | 8 |
| 424 | | 8.5 | 4 | | 2 |
| 425 | | 12 | 8 | | 2 |
| 526 | | 8.3 | 4 | | 4 |
| 427 | | 4.4 | 4 | | 2 |
| 428 | | 15 | 32 | | 4 |
| 429 | | 11 | 4 | | 2 |

NA: Not available
NI: No inhibition

TABLE XIV
MIC of selected compounds against a range of bacteria
A. Gram-positive bacteria

| | Organism: | | | | | | | |
|---|---|---|---|---|---|---|---|---|
| | | S. aureus | S. aureus | | | | | |
| | S. aureus | MRSA | USA300 MRSA | E. faecalis | E. faecium | S. pneumoniae | M. phlei | M. fortuitum |
| | Strain: | | | | | | | |
| | ATCC | ATCC | BAA- | ATCC | ATCC | ATCC | ATCC | ATCC |
| | 29213 | 33591 | 1717 | 29212 | 700221 | 49619 | 11758 | 110 |
| | MIC | MIC | MIC | MIC | MIC | MIC | MIC | MIC |
| Cmpd | (μg/ml) | (μg/ml) | (μg/ml) | (μg/ml) | (μg/ml) | (μg/ml) | (μg/ml) | (μg/ml) |
| 120 | 2 | 32 | | 4 | 4 | 16 | | |
| 122 | 4 | 4 | | 4 | 2 | 8 | 2 | 2 |
| 139 | 4 | 8 | | 4 | 4 | 16 | | |
| 143 | 4 | 16 | | 4 | 4 | 16 | | |
| 163 | 4 | 4 | | 2 | 2 | 8 | | |
| 168 | 2 | 4 | | 2 | 2 | 8 | | |
| 170 | 2 | 4 | | 2 | 2 | 8 | | |
| 173 | 2 | 2 | 2 | 2 | 2 | 8 | 1 | 2 |
| 186 | 4 | 4 | | 4 | 2 | 8 | | |
| 199 | 8 | 8 | 4 | 8 | 4 | 16 | 2 | 2 |
| 202 | 16 | 8 | | 8 | 4 | 16 | | |
| 215 | 2 | >128 | | 2 | 1 | 2 | | |
| 216 | 4 | 4 | | 4 | 2 | 8 | | |
| 225 | 2 | 2 | 2 | 2 | 1 | 8 | 1 | 1 |
| 232 | 4 | 4 | | 4 | 2 | 8 | | |
| 237 | 2 | 4 | 4 | 2 | 2 | 8 | 2 | 2 |
| 245 | 4 | 4 | 2 | 4 | 2 | 8 | 2 | 2 |
| 266 | 4 | 2 | 2 | 2 | 2 | 8 | 2 | 2 |
| 274 | 2 | 2 | | 2 | 2 | 8 | | |
| 348 | 4 | 4 | 4 | 2 | 2 | 8 | 4 | 4 |
| 357 | 2 | 2 | 2 | 2 | 1 | 8 | 4 | 4 |
| 360 | 4 | 2 | 2 | 2 | 2 | 8 | 4 | 4 |
| 371 | 4 | 4 | 4 | 4 | 2 | 8 | 4 | 4 |
| 372 | 2 | 4 | 2 | 2 | 2 | 8 | 4 | 4 |
| 381 | 2 | 2 | 2 | 2 | 1 | 8 | 4 | 4 |
| 385 | 2 | 2 | 2 | 2 | 2 | 8 | 4 | 2 |

B. Gram-negative bacteria

| | Organism: | | | | | | | | |
|---|---|---|---|---|---|---|---|---|---|
| | E. coli | E. coli | K. pneumoniae | H. influenzae | A. baumannii | P. aeruginosa | P. aeruginosa | N. gonorrhoeae | H. pylori |
| | Strain: | | | | | | | | |
| | | JW5503 | | | | | | | |
| | ATCC | (efflux | ATCC | ATCC | ATCC | ATCC | NTUH974 | ATCC | ATCC |
| | 25922 | defective) | 43816 | 49247 | 17978 | 27853 | (MDR) | 700825 | 43504 |
| | MIC | MIC | MIC | MIC | MIC | MIC | MIC | MIC | MIC |
| Cmpd | (μg/ml) | (μg/ml) | (μg/ml) | (μg/ml) | (μg/ml) | (μg/ml) | (μg/ml) | (μg/ml) | (μg/ml) |
| 120 | 32 | 16 | 64 | 16 | 64 | 64 | 64 | 2 | |
| 122 | 4 | 4 | 4 | 4 | 8 | 16 | 16 | 2 | |
| 139 | >128 | 32 | >128 | 16 | >128 | >128 | >128 | 2 | |
| 143 | >128 | 64 | >128 | 16 | >128 | >128 | >128 | 2 | |
| 163 | 4 | 4 | 4 | 4 | 4 | 16 | 16 | 2 | |
| 168 | 4 | 4 | 4 | 4 | 4 | 16 | 16 | 2 | |
| 170 | 4 | 2 | 8 | 4 | 4 | 16 | 32 | 2 | |
| 173 | 4 | 4 | 4 | 4 | 4 | 16 | 16 | 2 | |
| 186 | 4 | 2 | 8 | 8 | 4 | 16 | 32 | 2 | |
| 199 | 32 | 4 | 16 | 8 | 8 | 32 | 64 | 4 | |
| 202 | 128 | 16 | 64 | 16 | 32 | 32 | 32 | 4 | |
| 215 | >128 | 64 | >128 | 16 | >128 | >128 | >128 | 1 | |
| 216 | 8 | 4 | 128 | 4 | 8 | 64 | 64 | 2 | |
| 225 | 2 | 2 | 2 | 4 | 2 | 8 | 16 | 2 | |
| 232 | 4 | 4 | 8 | 8 | 8 | 32 | 32 | 4 | |
| 237 | 4 | 4 | 4 | 8 | 8 | 16 | 16 | 2 | |
| 245 | 4 | 4 | 8 | 8 | 4 | 16 | 16 | 2 | |
| 266 | 4 | 4 | 4 | 4 | 8 | 8 | 16 | 2 | |
| 274 | 4 | 4 | 8 | 4 | 8 | 8 | 16 | 2 | |
| 348 | 2 | 2 | 2 | 4 | 4 | 8 | 8 | 2 | 16 |
| 357 | 2 | 4 | 2 | 2 | 4 | 4 | 4 | 2 | 16 |
| 360 | 2 | 2 | 4 | 2 | 4 | 8 | 8 | 2 | 8 |
| 371 | 2 | 2 | 4 | 4 | 4 | 8 | 8 | 2 | 16 |
| 372 | 2 | 2 | 2 | 4 | 4 | 8 | 8 | 4 | 16 |
| 381 | 2 | 2 | 4 | 2 | 4 | 4 | 8 | 2 | 8 |
| 385 | 4 | 2 | 4 | 2 | 4 | 4 | 4 | 2 | 8 |

1. A compound of formula F-I: 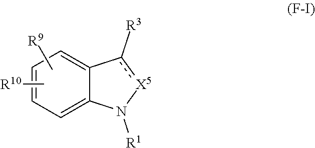
or a pharmaceutically acceptable salt thereof
wherein
X⁵ is selected from CH, CMe, C═O, and N; [symbol]
denotes a double bond when X⁵ is CH, CMe or N, and a single bond when X⁵ is C=O;
R¹ is selected from the group consisting of
R², (CH₂)ₘ—R², —C(O)—R², and —CHMe-R²;
R² is selected from the group consisting of
phenyl optionally substituted with one of more groups selected from -halo and —C₁₋₃ alkyl,
C₃₋₁₀ cycloalkyl wherein the cycloalkyl group is mono-, bi- or polycyclic and is optionally substituted with one of more groups selected from —F and -Me,
C₁₋₁₀ alkyl wherein the alkyl group is straight or branched,
C₂₋₁₀ alkenyl wherein the alkenyl group is straight or branched, and
heterocyclyl wherein the heterocyclyl group is a 5- or 6-membered aliphatic heterocycle;
R³ is selected from the group consisting of
CH(R⁴)—(CH₂)ₙ—C(O)NR⁵R⁶,
CH(R⁴)—(CH₂)ₙ—NHR⁵,
CH(R⁴)—(CH₂)ₙ—NR⁵R⁶,
CH(R⁴)—(CH₂)ₙ—CH(NH₂)—C(O)NR⁵R⁶,
C(O)—NR⁵R⁶,
(CH₂)ₙ-Cy-NR⁵R⁶, and
CH(R⁴)—(CH₂)ₙ—OR⁶;
R⁴ is selected from the group consisting of
C₁₋₆ alkyl, wherein the alkyl group is straight or branched,
C₃₋₆ cycloalkyl,
phenyl optionally substituted with one or more groups selected from -halo, —C₁₋₃ alkyl,
C₁₋₃ perhaloalkyl, —C₁₋₃ alkoxy, —C₁₋₃ perhaloalkoxy, and -hydroxyl,
benzyl, optionally substituted with one or more groups selected from -halo, —C₁₋₃ alkyl, —C₁₋₃ perhaloalkyl, —C₁₋₃ alkoxy, —C₁₋₃ perhaloalkoxy, and -hydroxyl,
heterocyclyl wherein the heterocyclyl group is a 5- or 6-membered aliphatic or aromatic heterocycle, optionally benzo-fused, and optionally substituted with one of more groups selected from -benzyl, -halo, —C₁₋₃ alkyl, —C₁₋₃ perhaloalkyl, —C₁₋₃ alkoxy, —C₁₋₃ perthaloalkoxy, and -hydroxyl;
R⁵ is selected from the group consisting of
H,
benzyl, optionally substituted with with one of more groups selected from -halo and —C₁₋₃ alkyl,
C₁₋₆ alkyl,
acetyl,
CN, and
(CH₂)₃—NH₂;
or
R⁴ and R⁵ together with the atoms to which they are bound form a heteroaliphatic ring;
R⁶ is selected from the group consisting of
C₁₋₃ alkyl, optionally substituted with one or more R⁷ groups
C₀₋₃ alkyl-cycloalkyl, wherein the cycloalkyl group is a 3-6 membered monocyclic cycloalkyl optionally substituted with one or more R⁷ groups,
C(O)-cycloalkyl, wherein the cycloalkyl group is a 3-6 membered monocyclic cycloalkyl optionally substituted with one or more R⁷ groups,
C₀₋₃ alkyl-heterocyclyl, wherein the heterocyclyl group is a 5- or 6-membered aliphatic or aromatic heterocycle, optionally benzo-fused, and is optionally substituted with one or more R⁷ groups,
C₁₋₃ alkyl-phenyl, wherein the phenyl group is optionally substituted with one or more R⁷ groups,
C(O)—(CH₂)ₚ—NH—(CH₂)ᵣ-phenyl, wherein the phenyl group is optionally substituted with one or more R⁷ groups;
or
R⁵ and R⁶ together with the atom to which they are bound form a heteroaliphatic ring optionally substituted with one or more R⁷ groups;
R⁷ is selected from the group consisting of -halo, —C₁₋₃ alkyl, —C₁₋₃ alkoxy, phenyl, hydroxy, —CH₂OH, -oxo, —C(O)Me, —SO₂Me, —SO₂Ph optionally substituted with —F, mono- or di-C₁₋₃ alkyl amine, —C(O)—NH₂, —NH—C(O)—NH₂,—C(═NH)—NH₂, —NH—C(=NH)—NH₂, —(CH₂)ₛ—NH₂, piperidine, piperazine, morpholine, —(CH₂)—NH—P(O)(OEt)₂, —C(O)—NH—R⁸, and -phenoxy optionally substituted with —Cl;
R⁸ is selected from the group consisting of —OH, -(amino)cyclohexyl, -pyrrolidinylethyl, and -methylpiperazinylethyl;
R⁹ and R¹⁰ are each independently selected from the group consisting of —H, -halo, —C₁₋₃ alkyl, —C₁₋₃ perfluoroalkyl, —C₂₋₃ alkoxy, —C₁₋₃ perfluoroalkoxy, —NO₂, —OH, —CN, —CO₂H, —CO₂Me, —CO₂NH₂, —CH₂NH₂, -Cy, -pyridinyl, -tetrahydropyridinyl, -pyrazinyl optionally substituted with -Me, and -phenyl optionally substituted with -halo, —C₁₃ alkyl, —C₁₋₃ perfluoroalkyl, —C₁₋₃ alkoxy, —C₁₋₃ perfluoroalkoxy; and
wherein m, n, p, r, s and t are each independently selected from 0, 1 and 2.

2. A compound according to claim 1, having formula F-II: 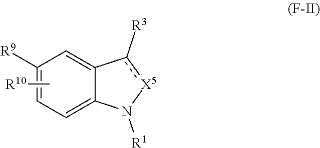
or a pharmaceutically acceptable salt thereof
wherein
R² is selected from the group consisting of
phenyl optionally substituted with one of more groups selected from —F and -Me,
C₃₋₁₀ cycloalkyl wherein the cycloalkyl group is cyclopropyl, cycloheptyl, bicycloheptyl or adamantanyl, optionally substituted with one of more groups selected from —F and -Me,
C₁₋₁₀ alkyl wherein the alkyl group is ethyl, isopropyl or octyl,
C₂₋₁₀ alkenyl wherein the alkenyl group is straight or branched, and
heterocyclyl wherein the heterocyclyl group is piperidyl or hetrahydropyranyl;
R³ is selected from the group consisting of
CH(R⁴)—(CH₂)ₙ—C(O)NR⁵R⁶,
CH(R⁴)—(CH₂)ₙ—NHR⁵,
CH(R⁴)—(CH₂)ₙ—NR⁵R⁶,
CH₂—CH(NH₂)—C(O)NR⁵R⁶,
C(O)—NR⁵R⁶,
Cy-NR⁵R⁶, and
CH(R⁴)—(CH₂)ₙ—OR⁶;
R⁴ is selected from the group consisting of
C₁₋₆ alkyl, wherein the alkyl group is straight or branched,
C₃₋₆ cycloalkyl selected from the group consisting of cyclopropyl, cyclopentyl and cyclohexyl,
phenyl optionally substituted with one or more groups selected from —F, —C, -Me, -iPr, —CF₃, —OMe, OCF₃,
benzyl, optionally substituted with one or more methyl groups,
heterocyclyl wherein the heterocyclyl group is imidazolyl, thiazolyl, pyridinyl, piperidinyl, tetrahydropyranyl, quinolinyl or isoquinolinyl, and is optionally substituted with one of more groups selected from -benzyl, and -hydroxyl;
R⁵ is selected from the group consisting of
H,
benzyl, optionally substituted with with one of more groups selected from —F and -Me,
C₁₋₂ alkyl,
acetyl,
CN, and
(CH₂)₃—NH₂;
or
R⁴ and R⁵ together with the atoms to which they are bound form a 6-membered heteroaliphatic ring;
R⁶ is selected from the group consisting of
C₁₋₃ alkyl, optionally substituted with one or more R⁷ groups
C₀₋₃ alkyl-cycloalkyl, wherein the cycloalkyl group is cyclopropyl, cyclopentyl or cyclohexyl, optionally substituted with one or more R⁷ groups,
C(O)-cycloalkyl, wherein the cycloalkyl group is cyclopropyl, cyclopentyl or cyclohexyl, optionally substituted with one or more R⁷ groups,
C₀₋₃ alkyl-heterocyclyl, wherein the heterocyclyl group is pyrrolidinyl, pyridinyl, imidazolyl, thiazolyl, piperidinyl, furanyl, benzodioxolanyl, oxazolyl, morpholinyl or tetrahydropyranyl, and is optionally substituted with one or more R⁷ groups,
C₁₋₃ alkyl-phenyl, wherein the phenyl group is optionally substituted with one or more R⁷ groups,
C(O)—(CH₂)ₚ—NH—(CH₂)ᵣ-phenyl, wherein the phenyl group is optionally substituted with one or more R⁷ groups;
or
R⁵ and R⁶ together with the atom to which they are bound form a 6-membered heteroaliphatic ring which ring is optionally substituted with one or more R⁷ groups;
R⁷ is selected from the group consisting of methyl, fluoro, bromo, phenyl, hydroxy, —CH₂OH, -oxo, methoxy, —C(O)Me, , —SO₂Me, —SO₂Ph optionally substituted with —F, —NH₂, —NHMe, —NMe₂, —C(O)—NH₂, —NH—C(O)—NH₂,—C(═NH)—NH₂, —NH—C(═NH)—NH₂, —(CH₂)ₛ—NH₂, piperidine, piperazine, morpholine, —(CH₂)ₜ—NH—P(O)(OEt)₂, —C(O)NH—R⁸, and phenoxy optionally substituted with —Cl;
R⁸ is selected from the group consisting of —OH, -(amino)cyclohexyl, -pyrrolidinylethyl, and -methylpiperazinylethyl;
R⁹ is selected from the group consisting of —H, —F, —Br, —NO₂, —OH, —CN, —CO₂H, —CO₂Me, —CO₂NH₂, —CH₂NH₂, -Cy, -pyridinyl, -tetrahydropyridinyl, -pyrazinyl optionally substituted with -Me, and -phenyl optionally substituted with —Cl, -Me, —CF₃, —OMe or —OCF₃;
R¹⁰ is —H or —Br; and
X⁵, R¹, m, n, p, r, s and t are as defined in claim 1.

3. A compound according to claim 1, having formula F-III: 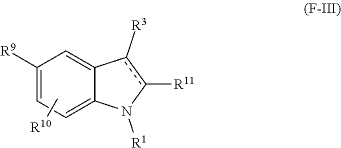
or a pharmaceutically acceptable salt thereof
wherein R¹¹ is —H, -Me or -oxo; [symbol]
denotes a double bond when R¹¹ is —H or -Me, and a single bond when R¹¹ is oxo.

4. A compound according to claim 1, having a formula F-IV: 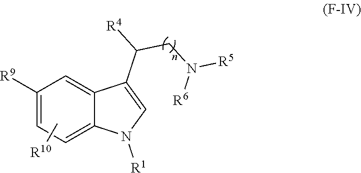
or a pharmaceutically acceptable salt thereof.

5. A compound according to claim 1, having formula F-V: 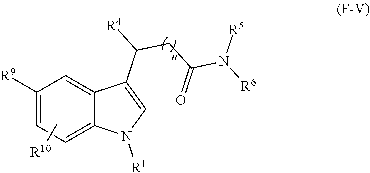
or a pharmaceutically acceptable salt thereof.

6. A compound according to claim 1, having formula VI: 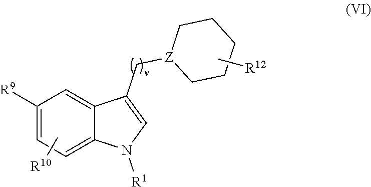
or a pharmaceutically acceptable salt thereof,
wherein v is 0 or 1,
Z is selected from CH or N,
and wherein
whenever Z is CH, R¹² is —NR⁵R⁶, and
whenever Z is N, R¹² is selected from an R⁷ group comprising at least one N atom.

7. A compound according to claim 1, wherein
R¹ is cyclohexanyl or n-octyl;
n is 2;
R⁴ is selected from the group consisting of -Cy, -PhOCF₃ and pentan-3-yl;
R⁵ is H;
R⁶ is —(CH₂)₃—NH₂ or -Cy-NH₂;
R⁹ is —H or —CN; and
R¹⁰ is H.

8. A compound according to claim 6, wherein
R¹ is cyclohexanyl or n-octyl;
R⁹ is —H or —CN; and
R¹⁰ is H.

9.-13. (canceled)

14. A method of treating or preventing an infection in a human or animal which comprises administering to a subject in need thereof a therapeutically effective amount of a compound according to claim 1 .

15. The method according to claim 14, wherein the infection is a bacterial, fungal, or parasitic infection.

16. The method according to claim 15, wherein the infection is a bacterial infection caused or complicated by bacteria of a genus selected from Staphylococcus, Enterococcus, Streptococcus, Pseudomonas, Legionella, Kebsiella, Haemophilus, Neisseria, Listeria, Escherichia, Helicobacter and Mycobacterium.

17. The method according to claim 16, wherein the bacterial infection is caused or complicated by a bacterial species selected from the group: S. aureus, E. faecalis, E. faecium, S. pneumoniae, E. coli, K. pneumoniae, H. influenza, A. baumannii, P. aeruginosa, P. aeruginosa, N. gonorrhoeae, H. pylori, N. meningitides, L. monocytogenes, L. pneumophila, M. bovis, and M. tuberculosis.

18. A method of inhibiting bacterial RNase P activity comprising administering a compound according to claim 1, or a salt thereof.

19. A method of administering a compound according to claim 1, or a salt thereof, as a bactericide.

20. A pharmaceutical composition comprising a compound according to claim 1, or a pharmaceutically acceptable salt thereof, in association with a pharmaceutically acceptable excipient, adjuvant, diluent and/or carrier.
